SEQUENCE LISTING (36 sequences)

SEQ ID NO: 1 - DNA, 98059 bp, synthetic construct
  source 1..98059: mol_type = other DNA
ttggctatcctaagcagccgattatgcgttgaggctttcagtgatggctgatatagcaacaatgagaatgaaggctctgcacttctgggataaacacggtatttctgcagcttctgaagcctttggcgtatcctgccgcacgctttactggtggcgtcagttactgaacaagggaggacctgaggggctaatcccgcacagtaaggcacctctggtgcgacgaaaaaagcactggcatcccgatgtgctgaaagagattcgacgcctcaggacagagctgccgaacctcggtaaagagcagatttttgttcgcctgaagccctggtgcgcataacgccatcagacctgcccgagtgtttccaccatcggcagaatgatcgccaccgcacacgataaaatgcggatgataccagtacgtctgagctcgcggggtaaggctctgcttgtcaaaaagcgaaccaccaaacccccgcaggccaaagcactaccgtccggtaaagacaggagagctcattgggatggacgccattgagctcagaatgggtgaaatgcgtcgctatatcattaccatgatcgacgaacacagcgattacgtactggcgctggctgtgccgtcgctcaacagtgatatcgtcaatcactttttcagccgtgcagcccggctgttccctgtcggtatcagccagatcatcacagataacgggaaagagttcctgggaagctttgacaaaacgctgcaggaagccgccatcaaacacctctggacctatccctacacgccaaaaatgaacgctatctgtgaacgttttaaccgggcattaagagagcaattcattgaatttaatgaaattttactctttgaggatctggcgttgtttaacctgaaactggcggaatatctggcgctgtataacagcaaaagactccacaaggcgctcgcactaacgacgcccgtggaatatattttaaaagagaacaagaattgcaatatgtggtggacccatacacagcaaacgcacaggccgccccggttgggtagatatgtgggatggctggttcagacttcaggatatggttgaggctggtgatgaagtctctttatcaggagatctgatcaagagacagccgcgcagggggattcactatgctggattcttttaacaaattctccgctaaccaaaaaatagactttttgcattcgtaaatgcgttaagagataatttttaaagttccatcaatctcccggccagttcgtctcgcggcattactcactatccgcacaacttcagtaagatcatgactttttcaatcgttatctgttgaccaagctttgaaaccactagcgtcccgtcaacctgacctggaatcgtcgagaaccgcccatagttgtgaattggctagagtgtgatggaaagcaacgctaaacgacctagtcacacattctcttttccctaacaatgaccatttagtgaaataacgtatccaaaatgatgacgtgaaagcctgacactggcagcgaaaagccgaaaaaactcacctggtcaggagcaaaaatcggattattcgccaacagcgtaatatcagcagtagttctcgacacgcttgcgctatagacagaggatgaaacgctgctggtcggagttctctatacatttttggcgacggcagaaccgctcaccaatgccttaacgtctatcagccttccggtgagttggaacagcatgctcagcgtttgcaagatattctccacggtctgtagcagcgtaggtttcaatttaaatatccccagatagccctctgggagcttccctttttattaagttttaattaccgttgcaatagttaaatgatattggtaattattatcattagcatttgtgtctgtgttgtttctggtaaggctgaaagggaacaaacatcgcctgtacaacggcgtttttacttacagaccagcgaaggcgagactatcaggatgatgcatcgtgtgtctaagaaatcacggttctttccagattcgttgttcggtctaacactgcacaaatgcgtcagaacatcccggcgtagctgtccgacgccatttgggctgttcaacaggacaatggcgcgtaagcgttaatggaagtggggatacggagttccctgttcacctgtgtatgtcacggaaaagtctctgactgagctgtccataacgctggtgaagcacaaatggcgcagcatctggtaaagcaggtttaacgaaaagtcgtgtcctgctgctggcatatgagtgcgtcgactgcctgatttagatcgtcaagcggagagggattttctcatgcgtattttgtttgtaggcccgcctctctatgggctgttgtatcctgtgctgtctctggcgcaggcatttcgcgttaacggtcatgaagtgctgatcgccagtggaggacaattcgcacaaaaagcggcagaggccgggttggtggtgtttgacgccgcacccggtctggattccgaagctggttaccgccaccatgaggcacagcggaaaaaaagtaatatcggaacccaaatgggcaacttctcattcttcagcgaagaaatggccgaccatctggtggagtttgccggacactggcggcctgacctcatcatctaccctccgctcggagtcatcgggccgctgattgccgccaaatatgatataccggtcgtgatgcaaaccgtaggcttcggccataccccctggcatatcaagggtgtaacgcgttcgctcacggacgcctatcgtcgccacaatgtgggggccacaccacgtgatatggcctggatcgacgtaacaccgccgagtatgagcattctggaaaacgacggtgaacccattattccgatgcagtacgtcccgtataacggtggcgcggtgtgggaaccatggtgggagagaaggcccgagcgtaaacgtctgctggtgagtctcggcactgtcaaaccgatggttgacggccttgatctaatcgcctgggttatggattctgccagtgaggtcgatgccgagatcatcctgcatatttcagccaatgcgcggtccgatttgcgctcgctgccgtcaaatgttcgcctggttgactggatacccatgggtgtcttcctcaacggcgcagatggtttcattcatcatggcggagcaggcaataccctgacagcgctacacgctggtattccacaaatcgtcttcggccagggagccgatcgcccggtaaatgcccgcgttgttgccgaacgcggctgcgggattatccctggcgacgtcggcctgtcgagtaacatgatcaatgctttcctcaacaatcgctcacttcgcaaagcctctgaagaggtagcagcggaaatggcggcgcaaccctgcccaggtgaagtggcgaaaagcctgatcacaatggtacagaaagggtaaccactacaccttcatgataatcatatgccagcacccatccaggagtcatgcatccataacaatgcgggaccggagcgtgctggcttttttgcggaacgatccggccagtggatcgtattttacagacggaggaggcttaatgcctgcgaatcacactcccacaccggctcagtcatggatagttcgcctagcgcgcgtgtgctgggaacgtaagaaacttagtgtcatcgtggtggtagcgtcagtatcgactattttgctggctgcgctgacgccactgctgacaagacaggctgtgaatgacgcactggcgggcaatccggcccgcctgccgtggctggcctgcgggttactgttgatcgctttttttgatttcatcggtaactatgtgcgccgtggttatgccgggatgctctcactctgggtgcagcataccctcagaggacgggtattcgacagtattcagaaacttgacggcgcaggccaggatgcgctgcgcaccgggcaggtgatttcacggaccaacagcgatctgcagcaggtgcataccctgctgcagatgtgcccggtgccgctggcagtgttcacttattacattgccggcattgccgtgatgctgtggatgtcccctgccatgacgcttatcgtcgtgtgcgtactggtatgcctggcgatcaccgcgcttcgtgcgcgtcgtagggtcttcgcgcaaaccgggctggcctcggaccaactggcgaatctcaccgaacatatacgcgaggtgctggcacagatctcagtggtaaaatcctgtgtggcagagatgcgtgaaacgcactggctcgataggcagtcgcggcagattgtgcgtgtacgcatcggtgcggttatctcgcaggcgatgcctggggccaccatgctggcgctaccggtgctcgggcaaatcgtcctgctgtgctacggcgggtggtcggtcatgcacgggcggatcgatctcggtaccttcgttgcattcgccagcttcctcgcgatgctgaccgggccaacccgcgtactggcatcgtttctggttatcgcacagcgcactcaggcgtccgtggagcgggtgtttgcactgatcgacacccgttcacagatggaggacgggacggagtcgattaacagtcaggttgtcggactggaactggagaatatgagctttgactaccaccatggcgacagacatatcctcagcgatatctccttttccctgcgcgccggtgaaaccgtggcggtggtgggcgcatcgggttcaggaaaatcgaccctgttgatgctgctggcgcgtttttatgatccctgctccggaaagatatggctcaacaccagcgaaggccgacaaaatcttcgcgatatcagactggaggcgcttcgtcgccgggtaggcatcgtatttgaagatgcttttctgtttgccggtacggtggcggaaaatatcgcctatggccaccctcaggcaacggcggacgacattcgccgtgcggcagctgctgcaggagccagcgattttatcaacgccctgccgaaaggcttcgatagcctgttaaccgaacggggtacgaatctttccggcgggcagaggcagcgaatagcgctggcgcgggcgctcattactgcaccggacgtgttaatcctggatgataccacctcagcggttgatgctgttacggaagcggagattaataccgcgctgggtcgctatgctgacgaagggcatatgctgctggtgattgcccgacggcgttcaacacttcagctagccagccgggttgtggtgctggataagggccgtatggtggataccggaaccccggcagaacttgaagcgcgctgtccggcgttccgcgcactgatgaccggcgacagcgattttctggccacgtcccacaatagccacaacgaattgtggccggctgaaccagcgacacaagacgatgtaacggatacgggggataaaggttttgtcgcccgtatgacccgcgtaccggaaaatgcagtacagcaggcgctggccggtaaaggccgcaaagtcacgtcactactgaagcctgtggcgtggatgttcgtcatcgccgctctgctgatcgcactcgattctgcggcaggcgtaggggtgctgatactgttgcagcacggcattgactccggtgtcgccgcaggcgatatgtcgaccatcggcctctgtgccctgctcgccctgagcctggtcattgtgggctggtgcagttattctctgcagacggtcttcgccgccagagcggcggaatcagttcagcattcggtgcgcttgcgcagcttcggccatatgctgcgtcttggactcccctggcatgaaaagcatgccgattcgcgtcttacccgcatgaccgttgatgtggactctctcgcccgctttctgcaaaacggccttgccggtgcggccaccagtctggtgacgatgttcgcaatcgccgccaccatgttctggctcgacccgttcctggcgctgacggcattaagcgcagtgccagtggccgcactggcaaccatgatttatcgccgcctcagtacccctgcttatgcacaggcacggctggaaataggcaaagtcaacagcaccctgcaggaaaaagtctctggcatgcgtgtcgtgcaatcgcatggtcagcaggaactggagggcgcccggctgcgcgcgttatcggagcgtttccgcgcaacccgtgtgcgagcacaaaaataccttgcagtctattttccgttcctgacattctgcaccgaggcctcctatgccgctgttctgttagtgggagcttcgcaggtcgccgctggagaaatgactgccggggtactggcggctttcttcctgttgctggggcaattctatgggccagtgcagcagttatcagggattgtcgacgcctggcagcaggcgacagccagcggcaaacatattgatgaactactggcgacagaaggcactgagaacctcggctcctcttcggtcctccctgtcaccggtgcactgcatcttgatgaggtcacgttcagttatcccgacagtcacgagccagctctgaacaaacttaccctgacgatcccagagggaatggttgttgcggtcgtcggtcgcagcggtgcgggtaagtcgacgctgattaagctgattgccgggttgtatttccccacgcacggcaacatcagaatcggtgtgcaaatgctcgatgatgcctcgctcgctgagtatcgtcgccagattgggcttgtcgatcaggatgtagcactgtttagtagtgatattgcagaaaacattcgttattcacggccatccgccaccaatgaagacgttgaaattgcctcacagcgggcagggctgtatgagatggtgtgcaatctgccgcagggattccggacaccagtgaataacggcggagccgatctgtccgcaggtcagcgccagttgattgcgctgtcccgcgcgcaactggctaatgctcacatcctgctgctcgacgaagccacgtcatgtctggatcgcacatccgaagaacgactgatgtcatcgttaacagatgtcgtgcatgccgggaagcactcggcgctgattgttgcacatcgtctgaccaccgcgcaacgctgcgatctgattgccgttattgataaggggttacttgcggaatacggaacccacgaacagctgttatctgcgggcggcctctatacccgcttatggcatgacagcgtcagcagtactgcgctccatcgccagcacaacatgaaggaggaaaccccgggatagttactggacacgtaatgtattaaaaacacagtcagaagcggcggtaccgtgaatagccgctttaattatttatactgacatccttaatttttaaagagtatgaatgctgaacatgcaacaacatccctctgctatcgccagcctgcgcaaccaactggcagcgggccacattgctaaccttactgacttctggcgcgaagctgagtcgctgaatgttcctcttgtgacgccagtcgaaggagcggaagatgagcgagaagttacctttctgtggcgcgcccgacatcctctgcagggcgtttatctgcgtctgaaccgggtgacggataaagagcacgtagaaaaaggaatgatgagcgcccttcccgaaacggatatctggacactgacactgcgtttacccgcaagttactgcggctcctattcgctgctggaaatcccccccggcactacggctgagacgattgcactgtccggaggccgtttttccaccctcgccggaaaagccgatccgctaaacaaaatgccggagatcaacgttcgggggaacgcaaaggaatcagtgctgacacttgataaagctcccgccctgtcggaatggaacggcggcttccacaccggacaactgcttacctccatgcgcattatcgccgggaaatctcgccagattcggctctatattccggatgttgatatttctcagcccctcgggctggtcgtgctgcccgatggtgaaacctggtttgatcaccttggcgtatgcgcggcaattgacgccgccataaataacgggcgcatcgtgcccgtggctgtactgggcattgacaacattaatgaacatgaacgcactgagatactcggcgggcgcagcaaactgataaaggatatcgccggacatctgctgccgatgattcgcgctgaacaaccgcagcgtcagtgggcagaccgttcgcgcacagtgctggccgggcagagcctcggcgggatcagtgcgctaatgggggctcgttacgcaccggaaacgttcggtctggtgctcagccactctccttcaatgtggtggacgccagaaagaaccagtcgaccaggcttgttcagcgaaaccgatacctcatgggtgagtgagcatctgctttctgccccaccgcagggcgtacgtatcagcctgtgcgtgggatcgctggaaggttcgacagtgcctcacgttcagcagcttcaccagcggctgattaccgctggcgtcgaaagccattgcgcaatttacaccggtggtcacgattacgcatggtggcgcggtgcactgattgacgggattggtttactacagggttgagttgacccacaaacactttcaggaaacggtacagacttcctgaataaatcaaattgtcacctgcggaaaaggaataatcatcagatgtatgcccgcgagtatcgctcaacacgcccgcataaagcgattttctttcatctttcttgcctcacccttatctgtagtgcgcaagtttatgcggagccggatatgcggccactggggccgaatatagccgataaaggctccgtgttttaccatttcagcaccacctctttcgactctgtcgatggcacacgccattatcgggtatggacggccgtgccgaatacaaccgcaccggcatcgggttacccgattttatatatgcttgacggtaacgcagttatggaccgcctggatgacgaactgctcaaacaattgtcagaaaaaacaccgccagtgatcgtggctgtcgggtatcagaccaacctccctttcgatctcaacagcagggcttacgactatacgccagcagcagaaagcagaaaaacagatctccactcagggcgttttagccgtaagagtggtggcagcaacaacttccgccagttactggaaacgcgtattgccccaaaagtggaacagggactgaatatcgatcggcaacgccgcggcttatgggggcactcctacggcggcctcttcgtgctggattcctggctgtcctcctcttacttccggtcgtactacagtgccagcccgtcgttgggcagaggttatgatgctttgctaagccgcgttacggcggttgagcctctgcaattctgcgccaaacacctggcgataatggaaggctcggcgacacagggtgataaccgggaaacgcatgctgtcggggtgctgtcgaaaattcataccaccctcactatactgaaagataaaggcgtcaatgtcgtattttgggatttccccaacttgggacacgggccgatgttcaatgcctcctttcgccaggcactgttagatatcagtggtgaaaacgcaaattacacagcaggttgtcatgagttaagccactaaacactgcccgcttttacgcgggcagtacgcctgaaacactacgatcagaatgatgcggtaactccggcatagtaagcccggcctggctcgttataggtattcgccccttcagaagatcggaagatctgtttattgaggatattactgacgccgacattaagacgcagatttttattaatatcgtaattgaagttcgtccccaccagtgaataagcgcccagctctttacctgacagaccgccagtatcttcactgcgggtttccgcatgagtacgcggtttttgtctgccatataacgtccagttgacgctggcagaaaacgcctgggtgatggtccagttaagcgagttattgatagtatatttcgggatgaccgacagaggattaccggtgtctttttgctccgaagtgatcatccatgtggcattggtattccagttcagacgatctttcaccagtgggaaagacatactggcttcgataccgtccaccagagctttcccgccattctgccacttgaggatatatgcgcctgaagcggtttgcccgataacgttatccccggccacgatcttattctggtaatcattgcggaagtaggtcacacttgcgtggtaatcttcccaggtgaactccagcccaatttctttattgacgctgatttccggatcgagatctttattaccgatcaggtagcacccgcctgatgtaatatcttttggacagccattgcctttcgagtagagcagatagccttcactggattgatacaggtttggggctttaaaggttcgggcaacccctgctttgactttgaaataatcgcccaattcctgcgaaagattcagactggggctgaagttcccgccggagtcgctgagataatcaaagcgcaggccgggaatgatattcgtgccaggaaccggctcaatgttatcttcaatatacagcgcactgatttgagaatgatttttactgctgcgatccgcagcagagccagaaataccgctgatatcactgtcattcaccgtcaggctggtagaggaaggatcatcgagcttatcgcggttccactctgcaccaacggtcagcgtttgatcaaccatcacattcaaaggaatattaagctcgccgctggttcgccaggaactcaggcgattggtcgtaaacttttcacccgctaaaatacgcccttcaccaccgccggataatccttcattcatgcgggtattattggttttctcgtaataaacaccaaagcgactttgtccccagtcccagataccattatgcgtaatgccataattctgtcggtacaggcggttcgtctctttgccggattttgccaggctttcggtaactgcactggaagaactgttttgcgtatcgcccgcatagatattcccctggcggctatatccggcttcgaaatcgagaatctgctgcggatttaatttccacgagacaacgccgttaatatctttgttacgtaccccttcatgcccggctgcgtttttcgtaccgaccggagaattaatatcccaactgtcagcatccgttttattcaggttaccatacaaacgcgtggtaagagcattaccagccagaggcccactaaggctgaaattggcgcgacgcgtagcgccctcatcgctactttccggctgattggtgtataacgacagcgaaccgtgccagtcgttggtgggacgtttggtaatgatgttcaccacccccccggctgcccccgaaccgtagcgcgccgccgcagggccgcggatcacttcaatacgctcaacctgttccggtggcacccagttggtgtcaccgcgggtatcacgctctccacgccagctataacgcacggagttacgtgacgtcaccggtacaccatcaattaaaattaaggtgttttccggccccataccacgaatatcgatctggcggttgttaccgcgtgtgcccgaggcgctattgccggtaagattgacgccaggcattttacgaataatatctgaaaggtcgtttaccggaggggtctttttaatatcctcgctggtaataaccgacacgcccggctgctgttttaatacctgctcagcggtggcttccaccaccagagtctcgtcattatcatcgtcggaggatttggctactgatacctggctattcaacccaaccaggagcacagttagcgaccagaggattttgttaattctcatacctattccctaataaatgcctaacttaaaatgtttgatcgttaagctcacatccttgccagatatttcttactgccattattgtttttatataagaatgataattaatatcatttagtaaaagaaaaagcaatccctcacaagataaatatattgatttttcataaatatcaaattgatatataacatatgttttttatttcattgcacttgtcgatcatcgggaattttgtcacaatttctcacggatagtgttcacattgtttctgacctgcatttcaggcgcgggcgctgcttatgtatataagggcctgtggtcgacgaatcagcataatcagatcttcctgcgaacaatttctttcagcgtatcaggcagcactatcggattatctacgagcgtttttgattcacattgtgtgtgaaacttcatacatcatgattcgctttccctactcagttctgatgattgttttttattctattaacacggagttaagattctaccctcggtgtcctcattattaatgaggaatgctacctatctaaaaactgaccgaaactttggggcatccctggaggtttccaaggagaaaaaggtgacagtgatagtgccacattgtgaggaaattcctgagaaactcctgagaaactcctggttcacacacataattcaggagcttgccatagatagtaaccaattgttttatattttaagaccattaaatacgcggctacgatagcgctttatataatcataaactgtagagataaaatatctgcttctctgataacactaattgagtgtcttaaactaagtaatttcatttttccccctttaaaagcaatatatgtgcaatagttgaagttaaagctgtgctgccagagttgataataattgttctgtctgttgccatgatatgcattcatcagtaacagactgaccatactctagcggaaaactatcggattgttttccatcttgtaaaaagctttccaccataatgccagctacatatcctggtatttttttccgattatctataacttgacgtgcaacggaaatttgccgtttagccactttaccgctattaccatggctacaatcaattatcagacgatggttaatgccctcatcatgcattaatttcactgccttagttatatcagacaaaccatagttaggttctctacctccacgtaatattagatgtccatgtggattgccatcggttaatagtgtagatatactatttgttaaagatgtcatatatacaatatgttgttcacgggctgcaataatagcgtcaatagctaagttaatatttccatcagtactgtttttaaatccaactggacaatgtaaaccagacgcaagttgacgatgcgtttgtgattcagtggttctggcaccaatagcaccccaacatattaaatcagcaatatagggagtaagaaaaggatcgaggaattctgtagcggttgcgacacgcatcgttgtgattgaagacaaacactggcgagcatatcgtatccctttctcaacatcataacttccatttagatcaggatcgtgcattattcccttccagcctttacgggttcgaggtttctcaaaataagtgcgcattacaatgtacattttcgataagtacttattctgtaatacgaacaatcgcttcgcataatcaactgcggcctgaacgtcatggattgagcatggaccaacaatcacaagtaagcgaggatcttttcccagcaaaatattcgctataatatctcgttgcagagagatccatgttacggtttcttccgatactgcgatttctttatgaatctcgcccacagtgggtaaactgccaagcaacttcccgcggtagattaatttactagctgactgcatcttgtctcctaaaagcgctatttcagaccatcagttgcacacagtagatgataatgataatcatttactgtataattggcaatatatggggattcatagtcatttctgagataatatacgttaggccttctctcccgtacggaaatcacttgttttccgtaaattacctgaatcttaatccctgagctgcggctagctgtggaactcacaaaagctgaccgggccgtggcacagcctctctgctggcagaacagcgtactgcagaagtcgtcgtactgccgctggtcccggcatcaggtctgcccgcagatggcacttctgccactgatctgccggtggagctgaacggcatgacatatcaggcctgccgtggggattttgtggtgcgcctcgacggcagcacctgtctgcagttatagaataaagaaggcggggaggtacgccgggagggcgatccgctggaagtggcgtagtggctgtaggcctgtaatgatgcaggaatagaagttcgtgtacaggttaatgaaagcatcaccccgtaaaaacatgccggcacggtttacacatcattccatggtggcagccggttaactttcctgcgccaccagtggaaagaggccatgagcagtccgaacaaaaaaccactcgtgatacttataataatggcctcacctgctaccattcctgacggcccccagtacataaaccacattgcacatccccaggaaataccccataagccccccatcagcagcgtaacctgccagaacggcataaagggcaatggcggaagccggatacccagccgccagagaatacgcagcagaggaggagcataattactccgccacatctttttgctgtccatcagggcaattgcccgggctttttttgctcaaaagtcacaggagcacctccgttccatgatgtaaacgtgacacaacttattgttattgattattattatcataccgtgattatgttgtcatgcacctgtacactgataaaagaaagggagaacaggaggctggctgaacaaccaatccgttcgtccagttcaatggcggctatcaggttctgctccgcaatgtactcacgaatgcgcttcctgtcctggctggccagcatggtccagaatatctccatcatttaccggcgactttcctgctcatttcatcccgccatgcagcagcggaagattcgaccatgtcatggggaagcacatcgccgcgctcgagttgctgacgtcctgcctcaacctggtcacggaaccaggtattatgctgccgttccacggtctggcgcataaaatcccggattaactgagagccattacggtccatgctttttgcagggccataaaagcatccttcagttcagcgtcaattcttaaactcatattaacctgtgccatttatcaccccgtcattaccacgtatatacacagtatataacgattcagatatcgtcactaggatatgccgcgccagcggcatggaaggcggcactccgctgtttcatatgataccgccgaagcccgatgtaagccgctacagtcgtccgaaagtcaccagcctcctcccccctgccgtcatccgtgcatcagctatgcactgagtatgcctgcccttccctagagaatcctgccaggcttgccacactgatatatcttgactttatgtaaacgatatgacactttaacatgataatgattaccattctcttttaatatacagagaaactaggaaatagatgaatgagttatgttactttaatattctctgacaataacctaaatcagttagattattgtcatttaataaataatgacattctttcatcataaataaaaagactattgtttataatattgttctcagcattatatgattatttatcctgataactctcctatgttgtatgtttatatgattttccttgaaacatataatgcaaattttcgatttattttccatcattaatccagataaacaacaaactaatagtatgcaaggagacattatttgtttcgccaggatgctttagaaaacagaaaaatgaagtggcagggacgggcaatattacttcccggaataccactgtggttaatcatgctgggaagcattgtgtttattacggcatttctgatgttcattattgttggtacctatagccgccgtgttaatgtcagtggtgaggtcacaacctggccaagagctgtcaatatatattcaggtgtacagggatttgttgtcaggcagtttgttcatgaagggcagttgataaaaaaaggggatcctgtttatctgattgacatcagtaaaagtacacgcaatggtattgtcactgataatcatcgccgggatatagaaaaccagctggttcgtgtgaacaacattatttcccgtctggaagaaagtaaaaaaataacgctagataccctggaaaaacaacgtctgcaatacacagatgcgttccgtcgctcatcagacattatacagcgtgcagaggaagggataaaaataatgaaaaataatatggagaattacagatactatcagtcaaaaggactaattaataaagatcaattaactaaccaagttgcattatattatcaacaacaaaacaaccttctcagtctgagcggacaaaatgaacaaaatgccctgcagataaccactctggagagtcagattcagactcaggcagcagattttgataatcgtatctatcagatggaactgcaacgactcgaattgcagaaagaactggttaacactgatgtggaaggcgaaatcattatccgggcgttgtctgacgggaaagttgactccctgagtgtcactgtagggcaaatggtcaataccggagacagccttctgcaggttattcctgagaacattgaaaactattatcttattctctgggtcccgaatgatgctgttccttatatttcggctggtgacaaagtgaatattcgttatgaagccttcccctcagaaaaatttgggcagttctctgctacggttaaaactatatccaggactcctgcgtcaacacaggaaatgttgacctataagggcgcacctcaaaatacgccgggtgcctctgttccctggtataaagtcattgcgacgcctgaaaagcagataatcaggtatgacgaaaaatacctccctctggaaaatggaatgaaagccgaaagtacactatttctggaaaaaaggcgtatttaccagtggatgctttctcctttctatgacatgaaacacagtgcaacaggaccgatcaatgactaacaggaatttcagacaaattataaatctgcttgatttgcgctggcaacgtcgtgttccggttattcatcagacggagaccgctgaatgtggactggcctgcctagcaatgatatgcggtcattttggtaagaatattgacctgatatatcttcgccggaagtttaatctctctgcccgtggagcaacccttgcaggaatcaatggaatagcggagcaactggggatggccacccgggctctttcactggagttggatgaacttcgagtcctcaaaacgccgtgtattctccactgggatttcagtcacttcgtcgttctggtcagcgtaaagcgtaaccgttatgtactgcatgatccggccaggggcataagatatatcagccgggaggaaatgagccgatattttacaggcgttgcacttgaggtctggcccggaagtgaattccagtcggaaaccctgcagacccgcataagtcttcgttcactgattaacagtatttacggtattaaaagaacgctggcgaaaattttctgtctgtcagttgtaattgaagcaatcaatctgctaatgccggtggggacacagctggttatggatcatgctattcctgcgggggacagagggctactgacgctaatttctgctgctcttatgttttttatattactcaaagctgcaacgagtacgctgcgcgcatggtcttcactggttatgagcacgctcatcaatgtacagtggcagtcggggctgttcgatcatcttctcagactaccgctggcgttttttgaacgccgaaaattaggtgatatccagtcacgttttgactcccttgacacattgagggccacatttaccaccagtgtgatcgggttcataatggacagcattatggttgtcggtgtttgtgtgatgatgctgttatacggaggatatctcacctggatagttctctgctttaccacaatttacatttttattcgactggtgacatacggcaattaccgacagatatcagaagaatgtcttgtcagggaggcccgtgccgcctcctattttatggaaacattatatggtattgccacggtaaaaatccaggggatggtcggaattcggggagcacactggcttaatatgaaaatagatgcgataaattcgggtattaagctaaccaggatggatttgctcttcggaggaataaatacctttgttaccgcctgtgatcagattgtaattttatggctgggagcaggccttgtgatcgataatcagatgacaataggaatgtttgtagcgtttagttcttttcgtgggcagttttcggaaagagttgcctctctgaccagttttcttcttcagctaagaataatgagtctgcacaatgagcgcattgcagatattgcattacatgaaaaggaggaaaagaaacctgaaattgaaatcgttgctgatatggggccaatatccctggaaaccaatggtttaagctatcgttatgacagtcagtcagcaccgatattcagtgctctgagtttatctgtagctccgggggaaagtgtggctataactggtgcttccggtgcgggaaaaaccacattaatgaaagtactatgtggactatttgaacctgatagcgggagggtactgataaatggtatagatatacgccaaattggaataaataattatcaccggatgatagcctgtgttatgcaggatgaccggctattttcaggctcaattcgtgaaaatatctgtggttttgcagaggaaatggatgaagagtggatggtagaatgtgccagagcaagtcatattcatgatgttataatgaatatgccaatgggatatgaaacattaataggtgaacttggggaaggtctttctggcggtcaaaaacagcgtatatttattgcacgagccttataccggaaaccaggaatattatttatggatgaggcaaccagtgctcttgattcagagagtgaacatttcgtgaatgttgccataaaaaacatgaatatcaccagggtaattattgcacacagagaaacaacgttgagaactgttgatagagttatttctatttaaaccatagaggaattacaagcgtatgaggaatatttcttcctgttataattcctcgttatgctcagatatctgttggaggtggaatggaagatagacaatccaccaagaagaaatatcattctgtgtggattgtccaataactgttctttcttatattaaataatactatttataaacaaacatcactaagattatttggactccaattacacaatcttcccgcagcatagttccatgcttctgaaggtatcccttcgggtttttgcttaattgttccccctaaaccggatggagacattgcaggattaggtttgtgagtggatgcatagtcatatattgcacctccagccacacccccagcagctgctccaattcctcctgcaacaaattgcccggatagtgttcctatagccatcgcaatatcacgccctgaagcaccaccagaaacagaatctaattcatttagagtcagagttctcatatgatctcctttttatcttatcggatattgaataataattatcaccaacaaagtaacatattgcagacattaatgcagagaagcaaaatgtatgcatggataaaaagtcctttcctctaaaaacacaatcatatatagctaatgcaatatatattgcggtggcatttattataaatgcaaataacaactctaattttgttctttttctatccattactttttatcccattactttctatcccattaccacacaaacactaacgataatgattatcgttaacatagtcaagagtgaagggtaggaggccctcaaccccctataaggggtccgcttggaaaacggatttccccacgtcaagagaattgatttgaacgagtggcttcgctacaaacagctcccgtttgtgtgtgaaaaacccctctcaacagatctcaactctgcacgatatctacaggagattgctccaaaaggcagtatcgtgtcttttaactaccgatataaccccgtcatcgacatgataatgcgtctgagaaatgagaagaaattaggtgaactgcatttcttctcagcagaatttaataaaaattctgcactgacccgccatcacctgacctggcgagactccgcacaacagagcaaaagcagcggtgctctgggcgatctatcctgtcatctgcttgatttattttgttttataggggaaagcccggtagtggttcatgggatcaaaacggtaaaggggacacgtgggtacaaaatcagatggtcaggttgaagtggatgataacggctatgtaatgggcagttcagaaaagggagcttatttcagagttcatgccagtaaatcggaaacagatcataatttgggactgcatatacagcttgtatttgaaaatggtgaaatcagatattcaacccaccatgaaaatcgcctgctcctgattctatttaatgatacgaatacggaaacaattggcttcgacgctctgaaacgcttgcctgatccaccacgggagcttccattctggtctgattcgtttattcatcttcatgacgactggtgtgctcttattaaatatggtcactcgtctcctaagctagcggacctgtcatccggccttcatatacaggaaataattgaggcattctgatatggataccattcttttaaccgggctgtttgctgcatttttcacaacgtttgcgttcgctccccagagcataaaaactatcaggacacgcaataccgaaggtatctccgttgttatgtatatcatgttcctgacaggtgtaatctcatggatagcgtatggcattatgcgatctgattttgccgtactgatcgccaatattgtcacactgtttctggctgcaccagtgcttgtaataacacttatcaaccgcaggaaaaaacacgtatgaatattgcaatcatcggcgcgggtcctgccggcatcatttctgcccgtaatgcgattaaagcaggtcactccgtggttctgtttgagaagaatacccgaattggggagatctggaacccctggagtggtggcgcttaccgtaatgcctgtatgcagaactctcgttatacatttcattatactggctttccccctggcgatatcgatgaatttctaggagtggaacaggtattcagatatctttcggccgtggccggagaggatgccctccgtgagtcgatccgactgaatactgaagttgtttcactcagaaaagacgccggacaccgggtgatccgctgcgcttctgaaggaaaagacacggaagacatttttgaccgggtcatcattgcaacaggtgaactctggcaaccccgccggccccccctgccaggtgaggaaaacttctccggaacgttgatcacgtcgagagattatcaggagccagaggcatttaaaggaaaaaatatcctcatcattggcggcggtgtcagcggtgcggatattgcctcagaccttgttccctttgccagaagcgtaagtctgtccgtcaaaaagatgggactttatctgccgagacaattcccgactggcccgaatgacatgatgcactcctatctgggcaggtgtctgctgagccaaatgaattatgaagattttatcggttatctcgacaccatgatgcctgactacatgcaggcctaccgagcatccagtctgttgcccgacatggcgaacaataacgcggtgcatgttaacgaaaaaatcataccgaatgtggccgccggtctgataaaagttaaaccccaggcagagcgtttcaccggagaaggcgctattaagttcgtcgacgcatctcaggaaaaatatgatgtaataataacctgtacaggctatgaaatgcctgattactcgttcattcgtaagcgtacagcctgaaccgtctggtcagaatctgacgaattagacaaagtggtgtccaccaaataagtagtgggaaccaaagtgtcagatatgcagaaaaatgtgactcccggcaggcgaaaaggctgccctaattatcctcccgaatttaaacagctgctcgttgctgcctcctgtgaacccgggatatccatctcaaaacttgctcttgaaaatggcattaacgccaatctgttgttcaaatggcgacaacaatggcgcgagggaaagctgctattaccttcttcagagagcccccagctacttcctgtgactctcgatgcagctgccgaacagccagaatcgcttgcagaggacccggaaaccctcagtatcagctgtgaggtaacgttccggcacgggacgctccgcttcaatggcaatgtcagcgaaaagctcctgactctgctgatacaggaactgaagcgatgatcccgttaccttccgggaccaaaatttggctggttgccggtatcaccgatatgagaaatggcttcaacggcctggctgcgaaagtacagacggcgctgaaagacgatcccatgtccggccatgttttcattttccggggccgcagcggcagtcaggttaaactgctgtggtccaccggtgacgggctgtgcctcctgaccaaacggctggagcgtgggcgcttcgcctggccgtcagcccgtgatggcaaagtgttccttacgcaggcgcagctggcgatgctgctggaaggtatcgactggcgacagcccaagcggctgctgacctccctgaccatgctgtaaatctctttatcctggttgtcacagaataagcccggtaaaatacgggcttatgaacgacatctcttctgacgacatcttcctgctgaaacagcgcctggccgaacaggaagcgctgatccacgccctgcaggaaaagctgagcaaccgggagcgcgaaatagaccatctgcaggcgcagctggataaactccgccggatgaacttcggcagtcgttccgaaaaagtctcccgccgtatcgcacaaatggaagccgatctgaaccggcttcagaaagagagcgatacgctgactggtagggtgtatgacccggcagtacagcgtccgttgcgtcagacccgcacccgtaagccgttccctgaatcactaccccgtgacgaaaagcgactgttgcctgcggcgccgtgctgcccgaactgcggcggttcactgagctatctgggcgaggataccgccgaacagctggagttgatgcgtagcgccttccgggttatccggacggtacgggaaaaacatgcctgtactcagtgcgatgccatcgtgcaggcacctgcaccttcgcggcccatcgagcggggtatcgccggaccggggctgctggcccgcgtgctgacctcgaagtatgcagagcacaccccgctgtatcgccagtcagaaatatacggccggcaaggtgtggagctgagccgttcactgctgtcgggctgggtggatgcatgctgctggctgctgtctccgctggaagaggcgcttcatggctatgtcatgactgacggcaaactccatgccgatgataccccggtccaggtactgctgccgggtaataagaagacgaagaccgggcggttgtgggcgtatgttcgtgatgaccgcaatgccgggtcagcgttggcacctgcagtgtggttcgcttacagcccggacaggaaaggcatccatccgcagactcatcttgcttgcttcagcggtgtgctgcaagcggatgcgtacgccgggttcaacgagctgtatcgcaatggtcggataacggaagctgcctgctgggctcatgcccgccgaaagatccacgatgtgcacgtccgcatcccgtcagcactgacggaagaagccctggagcagatcggtcagttgtacgccatagaggcggatataaggggaatgccggcagagcagcggcttgctgaacgtcagcgaaaaacgaaaccgctgttgaaatccctggaaagctggttgcgtgaaaagatgaagaccctgtcgcgacactcagagttggcgaaggcgttcgcgtacgcacttaaccagtggccggcactgacgtactatgcgaacgatggctgggtggaaatcgataacaacatcgctgaaaatgccctgcgggcggtcagtctgggtcgtaaaaacttcctgttcttcggctctgaccatggtggtggcggggagcgctactgtacagcctgatcgggacgtgcaaactgaatgacgtggatccagaaagctaccttcgccatgtgcttggcgtcatagcagactggccggtcaaccgggtcagcgaactgcttccgtggcgcatagcactgccagctgaataacacatccccgtcaatacggccctcgctgtacgcttaccaataaataatatgcttatcgacctgatggctgcaatgtcgcataaagactggctgagtcgtcgtcaacgacaaaaacagggaattgaacgtgcccatactttaggtaagtatcggggcaaacaggcagatcgggaacgacatcagaaagtaatgtactaccggcagatcaagaaactgagcatccgtgaaacggcagaggtgacaggctatagtacttcccaagtttgtcgcattcaggcgctctataaagaggttaaacctgattaaaaaatagccttaacgtacaatattttccgtttcccaaacggagcccaatacagatggctaacagaccgtctgcttccagttcaaatggtttaccgtcgcggtccagggcgcgaattttaactttctctgcgcgtggcactaaagctcgttgttcagggattgacaggcaaccttcttcaatgcctgtttcgccgcttttttctaaaagctccggattgattaacaccagccgttcgtcacggttttccgaaacatcaattacaatgatacgttgatggatatcaacctgggttgccgccaggccaatacctttttctgcgtacatcgtctcgaacatatcatcgacgatacgctgaatttctgcattcacttcttctaccggtttagcaactttgcgaagccgctcgtccggaatatgtaacacttgcaaaactgacataaatctccagagatgtgttcaggagttagaaagattatttcttctactgtaccgtaatcagataatcgcagcaaagggacaaagcaatagtccctttgctgcacagcatcagaatgaataacgaatattagcgtttatcgcatcatctgtgttgtatttaccaaatgcagagcgttcaacttccagccccaaacgcgtattgtcgccaaaccgggcatttaaccccacaccgtaaagcatacgactgtcttttctgccattaatctgatgttctcccgctgcatccttcagatgaacgtcagcactgtccgtcagatcgaactcataatgcaggccggcacgggctgtcagactccagtccttaccactgaaggttttaccggaaacaacgccggttctgcctaccagaggattaacgctgttacgacgcattgagacatccattccactgtcgttccagttaaatgtttggccctgcagtcttccccagaccagttccgcctgaggttcaacaaacgtcgtatctgtcagatgataacggtatccgacttctgcacctgcatacagtgaatggctgcggaagttctgtttaccagctccggcaaagttcaggtcatatttgttttcattgtgaatatatttggcaatcacatcaaagtaagcgccggaccggaacagaccactggcatagaaaccaccaccccatgattttgttttaccgctgtacaggtctgctgacgcatctgtgtcagtgtaggtggccatcacgccggtaaacaggtccatacttcccagttcgtgcttacggtcagcccccatctgcagcagggtatagtggtcagtgaaaccgccatcagcagagccggaaccgttcagcagacgcacccacgtaccggcttcgccattaatatccctcaaatcgcccatgcgtttgttcaggttgttaacttcagtgatgaagttgttatagctgatgtgcatgaatgtggcggcagccttaccctggccgtcgttacgtgcaacctggtaaccatcgaggacccactcttttttcccgtcctcttttctgacactaaggatgggggtgacatcactgaatcccacaacccgtgttgatgccctgaacagattatcagctgtcgcttcaggtgcgctgaccagtggaatatcaagcgtgtccttgttagaaggttttttcaggaagttaacccagatgctgttgtcatgacctgttgccgacttgtttatcaccagtttgtctgccttgttaaggtctgtacgcatgacaaatgctgactgaaccgcgtccagattatctgttgtcagtgtcgtgaacggtgatgttcccccgttaaaaccgactattgtccggttaagtttcatatttcctgccgtggagtttccgttcatcgaccactgggtgtctgtcatgctgacggtggcatccggtgcattcaggctcccgctccagatattgcggtacccgttaaacaggctgtacaacatctgattctgaagagtcaggtcaggactcagttccccttcccctccgagggtgacagtccctttatcctgaacattgatattacctgacaacatactgtacggccccacgttcaggcgggcatcgtctcccttcaggttccatgaaccggcataatcgtatacaggtaaaagtgtgtgagatacctcatcaggacggctgttcagactcagggcggcatcagaaatattcactggaccgtcagaaacaaaactctgactggccagagcatttgctcccttgttcagattcagggcggtactggtcagtgttgagttccccagaacggcactgtctgaggagatattcacggtactgttgttcgcctgtattccgccattgaatatatcattgatattcagcactgactgattatccaggttgacggtgccgttgaagacacttttatctgcatctttagttgcaacagatgtgccttcttcaagggtaaaggctgttccctggccatcgtttttgtcgataaatacccggctgtcgcccagcgtgacgctggagttatctgcctggatggttgtgttcagtgtggcattgcgaccaagaccaaaatcagtgttcttcagtgacagcctgtcaaaggtaaaactgcggttctcccagtcctcctgactgaatgacgtaggctgagtcagaaccgaatggtctccactggcagccagtttgtcagccacagactgagtattgtatgcatggataaccggatgcccctgcagcgtcagacgcccgttttcctgggtgaatgtaccggagatatccgcagctccgtccatcaccagagcaccggtaacgccttcaggcaggcgattgtccacattcagattgcctttcagttgtccgtgaaacagataccctgcagtattgaaacggtcagctaccagtttctgtgcatccccctgactgtgccccacaaactcccaggtggcgttgctgctctggtccgtggggaaataaccataggtgctctgcttcaggatgaagtaatccgtcgtatttgtgtacgggttattgtatttatataaatttccggcagttcctttacctgattccgaccagccattcagtgctactttgtcagcccgcagggcatagtccagcgtgatagtggcccgtttatcaacgttattcgccagcacggcaccataatctgccgccttcaactgatgaaacgtcagactgttaccattaacatccagtgtgcccccacgatatccccatgagacggtatccggatttacctgccgctcatcagtcagtaccacggtcggccggccactggcaatattaacactgctgaacgcctgcacctgtcctttattgtccgcctgctggttcagtacaacctttccgtccccgaccttcaggccaccttcattaatacctgtaccctgtaccgtcagcgtaccttcaccaattttatgcaggttatcgcccttaacaccattaacctgccagtttacggacaccccgttgtccacaacaataccggcaccggtccaggtacttccgttagaggttgttactgtgtaattatcacggaacgtcagggaacccgccccctgagaaaccgaatccttaaggttaatctgaccattctgcccctgaaatatcaggttcttaccagcattcaggtcatttccctgctgcccgtgcatggcatatgtggtggttccctgtgtcagcgcaccagctccggtactgctgttaaagctccactccagtgcaccaccttccgatgttctgaacgtgaccggggcatcattgtcttcattaaatttctgcccgataaaatccagtggaataacagcccagttatttcccctgccccccgcgccattccccgcagtaagaacaccgaccagcacccatttattctgaacagtatcaaaagcaaataaaggcgaaccgctgtcaccggcctcgccataaatgggcattgcaccattaagtttgtaatcaaaaaccagacctgaactggtgctaatcatttctccattctgataggatgacaggctaccgacagtcccgccggtcagccaggaatatgcacctcccatttgtgtcagctgtccgttactgtccttaatatactgagtaccagaccccagacgataaaaaacaggatagcgctccttatccagatatgcgccagccactgccccctgcgccgtcaccgcagtaggggcaacctctgtcaccagtttatccagccggggggcatgaaaatccagtgacggcgcattattccggtccacgatattgtaacggttttcaccatcaccaaagctaacgtttgtatatcccccgttatgtttcacgctggcgatatactgcgggtttatcagtgtcgccacaccgatttccgaatccacagcactgaaatcaggcatagctgccttatccagcgtaccgacaaattctccctgcttattataaatagcgatattcgttgcccccgggcggaacatccccttattttcagcaaaatcacgaaataactgatacccgagttcattattgaccgttcccgcaagacttcctgcagagaatagtaccgggatcagtaataaaaccgggaaacacagacgtctgacagacttatgaacacatttcctggcaaactcagatacggcaataaagccccgggccacagcgctgtagcgaagagaataaattctgttcatttttaattactcctgagaaaaataaaatcagtactactcaggcataacggataatcaccatcatcctgaggaatccccagaaaaaggagacaggcatagcgtgatgtgaactactgattctttcacaaattcagggagagcaccgttatgcctgcacataaagtatgccagaaccggtggcgaatgtgtgctatgcacaaatggtgaaacagttcctgagtcgtgacccgctcgaatgcgtgctgtgtggcggccgaatggtataccggcgtgccatagcggggctgaatgtggacgggctgaagaaaaacgcgcgggatatcagtctgataaggattactttccagccggctgatggctgacgggcgtaatcccattcgttcggccagcgcagactttgttaaccccgctttttctctcatttcgtataacatttctaccatctccagttctttgtcagcatcttcataccctttaacagcttctggagtattaagcagtttttgctttacctggctgaacggaacactttttactttcgtcatttcatctccttcaggcgcatccttgaagtggtttactgaatttggccacctgaacagaggtgatatgctcacctcagaacaacacaggtgctccaatgaaaaaaagaaatttcagcgcagagtttaaacgcgaatccgctcaactggttgttgaccagaactacacggtggcagatgccgccaaagctatggatgttggcctttccacaatgacaagatgggtcaaacaactgcgtgatgagcgtcagggcaaaacaccaaaagcctctccgataacaccagaacaaatcgaaatacgtgagctgaggaaaaagctacaacgcattgaaatggagaatgaaatattaaaaaaggctactgtagattcaattggtcaacgcaacagttatgtgaaaacatggggttgcggaggttttttgaatgagacgaacatttacagcagaggaaaaagcctctgtttttgaactatggaagaacggaacaggcttcagtgaaatagcgaatatcctgggttcaaaacccggaacgatcttcactatgttaagggatactggcggcataaaaccccatgagcgtaagcgggctgtagctcacctgacactgtctgagcgcgaggagatacgagctggtttgtcagccaaaatgagcattcgtgcgatagctactgcgctgaatcgcagtccttcgacgatctcacgtgaagttcagcgtaatcggggcagacgctattacaaagctgttgatgctaataaccgagccaacagaatggcgaaaaggccaaaaccgtgcttactggatcaaaatttaccattgcgaaagcttgttctggaaaagctggagatgaaatggtctccagagcaaatatcaggatggttaaggcgaacaaaaccacgtcaaaaaacgctgcgaatatcacctgagacaatttataaaacgctgtactttcgtagccgtgaagcgctacaccacctgaatatacagcatctgcgacggtcgcatagccttcgccatggcaggcgtcatacccgcaaaggcgaaagaggtacgattaacatagtgaacggaacaccaattcacgaacgttcccgaaatatcgataacagacgctctctggggcattgggagggcgatttagtctcaggtacaaaaaactctcatatagccacacttgtagaccgaaaatcacgttatacgatcatcctcagactcaggggcaaagattctgtctcagtaaatcaggctcttaccgacaaattcctgagtttaccgtcagaactcagaaaatcactgacatgggacagaggaatggaactgaccagacatctagaatttactgtcagcaccggcgttaaagtttacttctgcgatcctcagagtccttggcagcggggaacaaatgagaacacaaatgggctaattcggcagtactttcctaaaaagacatgtcttgcccaatatactcaacatgaactagatctggttgctgctcagctaaacaacagaccgagaaagacactgaagttcaaaatgtcaacgacggatgaaaagtgatccacttatatctccaccaacggcccaatattgatccaccgttttactcaggattagcttctgctataaccccggcctttcgtttctgtctgagtcgatagctttctcctttgatttgaacgacatgtgagtggtgtaagatacggtccagcatcgctgaggtcagtgctgcatcaccggcgaacgtttgatcccactgcccgaacggcagattggatgtcaggatcattgcgctcttttcgtaacgtttagcgatgacctggaagaacagctttgcttcttcctgactgaacggcagatagcctatttcatcaatgatgagcaggcggggggccattactccacgctgaagcgtcgttttataacggccctgacgttgtgccgtagataactgaagtaacagatctgctgctgttgtgaagcgaactttgatacctgcacggactgcttcatagcccatcgctattgccagatgggttttccccacacctgatggccccagtaatacgatattttcattacgttctatgaagctgagtgagcgtaacgactggagttgcttctgcggtgctccggtggcgaatgtgaagtcatactcttcgaacgttttcaccgccgggaaggctgccattcgggtatacatcgcctgtttacgttgatgacgtgccagtttttcttcatgaagcagatgctccaggaagtccatataactccattcctggtctactgcctgttgtgacagcgcaggcgctgcgcttataaggctttccagttgcaactgcccggcgagcaccatcagtcgttgatgttgcagttccatcatcacgccactcctctgcagaatgagtcgtagatggagagtggatgatgcagggggtgtttgtcgaagttcaccagattttcatcaagatgcacgtcatactcttttttctccggaggcagtgccagcatggactgctgctcttcgagccagcgatcgcagggacgtgcctggattgtttcatgctttcgttggttagcgacatcgtgcagccagcgcagaccgtggcggttggctgtttcaacatcgacagtgatccccatcgggcgcaggcgagtcattagtgggatgtaaaaactgttacgggtgtactgcaccatccgttccaccttacctttagtctgtgccctgaaggggcgacacagtcggggagagaagcccatctccttgccgaactgccacagcgaaggatggaaccggtgctgaccggtctgatatgcgtcacgttgcagaaccacagttttcatattgtcatacaacacttcgcgcggcacaccaccaaagaagcggaacgcattacgatggcaggtctccagcgtgtcataacgcatattgtcagtgaattcgatgtacagcattcggctgtatccgagaacagcaacgaacacgtgaagcggtgagcgaccattacgcatagtgccccagtcaacctgcatctgtcgtccgggttcagtttcgaaccgaacggcaggctcctgctcctgaggaaccgagagagaacgaatgaatgccctgagaatggtcattccgccacgatatccctggtctctgatctcgcgagcgattaccgttgccgggattttgtaaggatgagcatcggcgatgcgttgacgaatataatcccggtattcatccaggagtgaagcaacagcaggtcgcggcgtatattttggcggctcagattttgcctgcaaataacgtttaacggtattgcgggagatccccagttctctggcaatcgcccggctactcattccctgcttgtgcaggattttaatttccataactgtctcaaaagtgaccataagctctcctgaatcaggagagcagattaccccctggatctgatttcaggcgttgggtgtggatcactattgcaccgttcgtgacacaaaacaccgaaagagataattgaaaggggtgttgcattgacagattgaatctacaaccgcgctcttgatgtcagactccctcgagtaggcagcctggcggctgcggcttgtcatggcctggaattaccgttataaaaaaagataatgtcattgtctttcaggtagttatatggcccgttcagctaaaccccgtaaacgcaaacccgcaccacaaagaagcaaacttccccgctatgttgtgaaacttcatccggatgatttttttgacgaagaagacgctgaagttctgcgctttgataattttgacgatgccgttgagtgctgcgctgacctgggtattccgttctttctggatgcaggaaacaaaaagctggtcttctggtttgttcgtgtcgatgacgaagggtatccggaaatagcccgctgtacggagcgggagtttgcaaccattcttgccggtatcagtgccggtggtatgtactgcccggaatgcggcacagttcactggccggatggcgttaccccacccgtctgatgcttccccgttttgccgatatttttcagcagggtaaccgctggcttaactggctggagaaacagccggaaggttcagtgcgtccggtggtgactgagtcagtgacaaaaatcatggcatgcgggaccacgctgatgggctacacgcaatggtgctgttcgtcaccggactgttgccacaccaaaaagatctgcttccggtgtaaagccgctcctgtccgaactgcggggtgaaggctggcgcacagtggatacagtatctgctgagcctggtccccgactgcccgtggcagcatattgtgttcacacttccctgccagtactggtccctggtgttccacaaccggtggttactggcagagatgaaccgcattgcagcggatgtgatactggaaatctgccatcaggcagatgtggagccggggatattcacggtgatccacacatgggggagtgaccagcagtggcatccgcatatccatttatcgacaactgccggtggtgtgacgtcgggccacacctggaaaaatcttcatttatacgcccgtaaggtgatgagcatgtggcgctaccggataacgcggctactgtcccggaaatacccggagctggtaataccggatgaactggcagtggaaggaaacagcaaacgggactggaatcgcttcctggacacgcattaccgccgcggctggaatgtcaacatatccagggtgatggataacgccacacatgtggcggtgtacttcggctcttacctgaaaaagccaccggtgccgatgagtcggctggagcattatgccggtcaggatgaaatcggtctgcgttacaacagtcaccgtacaaaacgggaagaatacctgttgatgagtggagatgagttcatggaaaggttctcctggcatgtagcagataaggggttccgtatggtgaggtactacggtttcctgagtccggtgaagcgccgcttactggaagaagttgtgtacgtcataacggagacggtgagaaaaacggcgatgcaaatcaggtggagagggatgtatcagaggttaccgactggcagagctggtcctgatgcatgagcgactggcacgacagcaggtgtgcggctgagagtcgcagaggggaagttgcgtccattttaccggaaacggagcaaaaaaccgccattcataccctgtatcaatcagtgtcatcctgtttaatagtcgtttccgctcatatggtgcacaagaggtgttgaagaaatatctgttttgtggtgcttttttagtcttttggggatttaaattcctatcgatcaaggtcacgcatcagctttttatatgagcaaaatccaggctgcttttttatccattcagtatctatttttaaccttattaactttttattatgtgttccatttatagacgtagactcagatatattaccgactcttcgcataaatgatttttctgattcacttaatttttcagtaccgttacataatccgtgaccatatggtaatggagatgtcgttaaccatactttattatacaaaattgttccatcatacaacgttagatatccttctctcaggccttcagatgcaagtatggaatgcaacataggaattgctgtaaagtgatataaatccataaataaccctattctgtctatttatattcattttatatcatgttcaggccagactggcgagtcgtgcatagcctgtttactgaatgccgcttcagcggcatgtaaggcggcacgctgtggttacatgtgataccggagtaaaaccgccgaagcccggcgtaagccggtactgattgatagatttcaccttatccatccccagccctgtcagaccatacccgctttcagccatgacagagtttctgtgcgcggtcggagtggtcccgacgagggtttacccgaagtcggggcgtatctccgcgttagcgggccgtcagggccgcttacgagcgtgtattcaatacttccagccagaagactgacagcgacggcaatgcttagttacaacattcataattaaaagcgactctgttccggcccgaagggccggggcggggccgcttttcagttatgagggaggggctttgtggtttcagttctgcgctggttcggggtttttctggaggttggttttgtgtgttgtaactaaagtggctccggttggggcccgccgtttacggtgggaggtgcatatctgtctgtccacaggacaggcagtgaataggttttctttttaaatgaatgtaattaagtagtttaaaggagatataaacaggtgtttaaaagatacattgcaccctgtaaggctggcgcctggcgctttatgacatgaacggttgtaaccttatggggaagtccattgcagttaaatgtggataagcaaaattccccgtctgtgaggcgtgttttgtatcaaaaacaggggggggccagatgcaccagaaggtggatgatgaggttgttttttgtatgaggtgctgcttttttgttcgctggcaggcttcaggcgtgcgaatgcctccggtgcgggccggattattcagaagaggtcactttcagggggaagctgtggccagccggctgtaattacggttacgtgacagaatcatgcgctctttcacacgacgctccacttcgcgttttaccgcctcacgattggcagtgaagcgcccttccgagatttcacgcgtcagctgccgtttcaccagggtgacgatatcctgacgttccctgttcgcatcacgacgcgcacgggcacgctttattccacgggacttaagctctgtctggtaactgcggaaacgctcacgcacaaaacgccaggctttcgctatcagttcatccatacccagggtatccagcccctgctttttgcgctgtctgttttcccattcaacacgactgcggcgcgcagcaaccactgcatcctcagacacatcaagggcggcaaataacgccggtgtgaacgtgatatcggtcggaatgtagcacccgataagcggatcatattccgtctggtaggtaatcagtcccagctctgcaaggaacgtcaaggctcgggaggcccgggtgatggagagttttccggcagcagactccgtcgccagtccgcactcaatggccagcgtagtgatggagcactggacgcggttggccagcgggtcatagtgaaaacacagcccctgcagcagcgcatcaatagcccggcgacgcagtaccggtggcatgcgccgacgcagtcccttcgaacgggcatgtgccacatgaatggcgaaatcaaaacgggaggtgaagcccaccgccttttccatcagtttttcgcagaacttcagcgttccggcaccttcacggggtgtgaacaccggattcggatttttaacctgtgaatagtatttttgaaggagatcagccatgtccccctccaacactattccccctcctacgggcggtaactagctcgtctgggccgacacgtaaagtttcgaacatatctggtctgaaccagatggttcctccatcaaaagcgatgagatgaagatcattatcaagagttttgagggctctccaaacacgagtgtatgaacctccactacaaactgaaagctctttaacagataagttaactttgttcgtatcagggatgtagtgtgcgcacattacttgtacgagggaaacataaaaactaaccatcgacgcgctgattttatttttacgtttgccatgcgctattgcaagcaaacctctgatgaagatcagatgcttgaaggagtcaaaatgagcccttttctggagaaggctgcaaaacgggtgaggatacttgctggaatcaatgtgaagatctgattcaatcccacccgctattactgaatgtgtttcatgattagccacaccatcctgcacttacaatgcacagaaggagtgagcacagaaagaagtcttgaacttttccgggcatataactatactccccgcatagctgaattgttggctatgcggttttatggggccccggtaatctttctagtcgccaaacttgaagaagattatcggggttttcgcttttctggctcctgtaaatccacatcagaaccagttccctgccaccttacggcgtggccagccacaaaattccttaaacgatcagcaatctaccactcactcctgagataagcaagaatgtgaatatttacgaagtcgctctgcgtttcagctctgattcaatcagtttttcaagcatctgcgcctgagtaataccttcctcatccgccagttcactcagcctgtcttttaaccgggcctgaataaccgcatgaaaagccttatgagtgttgcttctgcgagccagagaagctctttgcctctctctggccggaacagggttacctttccggtatgtacgcttggtgcctgatgaggaagtcactgcattttctgtctgcgacatctcgcctcctcaattctcaaacagggatcgtttcgcagagaatactacagttttttgaaatcagcgacttgaaagttgtgacgatgatctgtgttggcgcacaaatcatgtctgattacgaccgtttaatgtgattttgataatgaaatgactgaaaagctggaaatgccttagccagggtgccggccccgggcgggaccggctgtttctggtatatagggtgtctgcccctgaaatacctcacacttccggaaagcggggacttccgtccccgcccggcagcttacttccgtgcttcgtaggccagagttacctgcaccactgtatttcctctgtgaatattcagctcacataaccgttccctgaatatcagtgaaaaacacaacaccgtggcgcacacggcgagcaacccgataagggcatatttcgtcataaactttgacctctcagcaaagaagaggctacaatcagtagtgttaaggtacttcatgcagacctcacgagttaatggattaacgagtggggtcttcgcatttctggtacctgcgtcttgcctgaaccagaaaggctcaaacacccgccgcgataataccacattccctgttgctgactattacccccatttgcctgtttttgttacaaaaacgccgggagcgggggaaaacttactttttcccgtttccgggggggacaactgagcattgcgaacgcgtcagctacgatgttccggtgattgctgatcagcccctgttgtattcataaactaattaacattcattctgtctggtgcataaatctgcccgtgaacatctgcagacaataccggccatgaaaagataaataataccagtggaatatatttcctcatgttattctccatatcagataaatggcacctgtatttcagcccccctgaatatcgcagcacacattgaataagaccttattttgtgaaattaatcacaaaatataaaatcgcgacttaatacggaaaaaactctgtgctactcagaactggccggtcatactgcccgaccggatattaaaaaaagcaaatctcataaactcctttcagccttctgactcctggagtaatgaaggctgcttttttcgttacacccttccccggcagcgccggtggcagtgttcagcccttttccgggcaaactgttgccagtctgtccggggagctatgcaggcagcccgtcgccatgcttctgccgcttccatccagcgtccactgacctccagaataacggcccgcccgccggcacgctggtaacgccagacctgaccgttccgtgaactgtttctgaatccattcatttttctgctcctcccttttagtcgtcagtattattgtggtaaataattcagtggactggcttcctttcgggtacggaagttatttccttttaagccttattgtttcagcctccccggtttccggggaggctttttttatttcccatcaagcacggcctgaagttctgcctttgtccggttctggcggcgaattttctccaaacgccctgccgcatacgcttcctcctcctgagaaatatgctccgtcacatacccttccgtgtcataccggcaggcaccggctttcatggcacacagatagctttctgaacgggtgatggccttcagcgcccggcgcagttttttatgcgagagcgggatattccgctgcgccacgtcttccagtaacacgtcccggataccgcaggccagaagccggggcgtgttaccgtcaaacaatcccggccaccaaggtttcagagtgttcaccgcctcatccagcgagggcagcgtcagataaatggacagcgcctgtctggcctgtgctttcttcgccgccagctctgcttcccgggcagccttctccgccagtttctgctttttcaccttccattttggtggcgtggtgacattgatgatggttttccggctccgggtcggtgtttccccttccgttttccgtttcagtgtcagtaccggtcgtttctgctctgtcatgattgccctgacctttgtgaaaatatgtcctccaggtcgttgaaactactcactatgatattaagctgacctcaacgacctctgacatattaataccgacaattaaagatgttttttaaaccattgcgtctgtactgccgccacacgttcaaaaaacgcgcctttataaatatcataatggcatgcgtcagcttcctcgtataattccttagtaccagaagcaacagcatcatataaggctcttccttgttctggtggattaacactatcctggccggcaataacaaccaaaactgggcactgtactttggctgcgctctctgccggtttatattgcaacgtctccataaccgtaaggaaaggtatttttatatccatttcaggatactgaccttttaccttctcgaagaatacctttgattcattatcactcagtacccgggtaacccccacaaacatctctttcccggtatttttcttcttttctgccattttattcagcgtggacagaaaagaagctttctccagttcattcatttctccggtaaccagaacctcaccgtcagcaaaagccagctggctgacaatacttttcactcttttatcctgcgcagctgcattgaatacatgacacccacccagcgatgttccccataatcctatacgctgattatcaatacatacctgcttttctgcccaattgataactgaaataatatcttcggtctgcattgccggaaccagacgacctcgttccccttcactctcaccgaatccacgataatcaaaagtaatcgttgcaaacccggcttctgtaaacgcattcgcaaaggaaggcagcagaacattccggataccgcaaaatccatggcataagagaatcaaaggatgttttatatttccttccgggacacgaaatgtcagggcaataccttcagggagtttgtgatccgtaattttcataaatatcctgtgttatgttgtttattaataagcagaatcagcgccagcatcataccagatttgccatcgtccacatatccacgggctggcaagggagcgcagcgaccgcgcagggcgaagcccggagagcaagcccgtagggcgccgcagccgccgtaggcggtcacgactttgcgaagcaaagtctagtgagtacgatataagttgttggaagatcttccctaaatttaaatataaacaacgaattatctccttaacgtacgttttcgttccattggccctcaaacccctaattaggatcaataaaacagcgacggaaatgattcccttcctaacgcaaattccctgataatcgccactggactttctgcttgcgcggtaaggcaggataagtcgcattactgatggcttcgctatcattgattaatttcacttgcgactttggctgctttttgtatggtgaaggatgcgccacaggatactggcgcgcatacacagcacatctctttgcaggaaaaaaacgctatgaaaaatgttggttttatcggctggcgcggagtggtcggctctgttctcatgcaacgcatggtagaggagcgcgatttcgacgctattcgccctgttttcttttctacctcccagtttggacaggcggcgcccaccttcggcgacacctccggcacgctacaggacgcttttgacctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcctagtactcaagcaaatcaaagcggtgtcggcgcgttgttgtagccgcgccgacaccgcttttttaaatatcataaagagagtaagagaaactaatttttcataactctctatttataaagaaaaatcagcaaaaacttgtttttgcgtggggtgtggtgcttttggtggtgagaaccaccaacctgttgagcctttttgtggagtgggttaaattatttacggataaagtcaccagaggtggaaaaatgaaaaaatggatgttagccatctgcctgatgtttataaatgagatctgccaggccactgattgctttgatcttgcaggccgggattacaaaatagatcctgatttactgagagcaatatcatggcaagaatcccgttaccgggttaatgccatcggtattaatccggtaacgggatatggcagcggactgatgcaggtagattcccagcattttaacgaactggcccgctacggaattaagccagaacatctgacaacagatccctgcatgaacatttataccggtgcttattatctggcaatagcatttaaaaaatggggcgtcacctgggaggccgttggtgcatacaatgccggattcaggaaaaccgaacgccagaaccagagacgtcttgcctacgcatcagatgtttaccggatttatactgggataaaaagcagtaaaggtatccggcttccggccacgaagaaatcacttccggaaattaacagcgtgcagaacaattaatcttacagacattgtcagataccggttatgccgcaaaagcggcatggaaggccacacacaaaaatatcatatgagtgacaatactgactggccgaagccccgcgaagcgggaacccatcttatatatacccggtaactgtgcagcctcctgccagaccatttttcgcgggcacaatatttacagacaaccggaaatccggttgatgaaattgaggtatatcagacgacacagcaatgccgaaaaatgataatataagtaaagccccgaaaattttttcggggctttacttatattatcatgacttccactttctgaactgttcggcaatcacccataacgccttgttgattcttatatcaccatcaatacctgtaatggctcttgtcctggtatttttcccggaggcacttcgccccgataatccaccttttatcatattctcctgaacccgctgccaggttgtccagagatcgttctgtttgtcttcccgacgacggggagtaattatctgttcaggtgtcactggcgttttattttcatcttcatacctgaccatcagtgcagctctgccaaataaatgttgctcgtcactgttaagatgaatttctttcatcgcctccatattatcagtgaccttatcaaagacaccgagcacttcatacgctccctcgataacctgcccgacaatatcacctttatgtggaacgcggatttcgccaaaattatttccgcacaccaggccatttgtgcagacaaaacgaaaaattccggggatcatctgataactggatgaaccatcatgtgaattaagcaggataatttcaggaacttcctgtccgttaatatgcccttccctgcgaagacgtaacatatgtttactgtattcgcgacgtcccaaatcacgaatccgactctgacaggcaaagaatggctggaaaccttcatcacgtaacttattgatgatattgattgttggaatatacgtataacgttcactccgggactcatgtttatcaccggaaaataccgaaggcacgaactgcattaattcatcatccgttaaaggacgttcacggcggatggaattataccgaccaaaacgggaagctaatcgcataacagctactcctttatgaaagaataaatttaataaaaaatttttccgcttaatatcatccccccgatctccattaacaagattgcaacaatcaggagggatattcatcacatccggtcagcagatcgcccggatatcgaggccggcacgcttaatctgacgcaggaactgtttgaagtgatcggcttccgcaatgttgatatggcggaactgcttatccacaccctgactgagcgcgtagctataactgaaggccatcttttccgggtggcttttgcgatattttttcagctcagtcaggctgctgaattccaggacgtcccactcttccccggtgttttttttcttcccggcaacaagaagcgtgtacgacgcggtgttaacgtggatatttcgggtactcatcctgctttctcctctctctggttttcagcccccatcatctgttgagtttcgggagccgcggttttgacgttttgcggggacctgccttttgttccctccggtattcaccatcacctgtttgagaaccttcgccgcatggggcatcactgccacggccggaaacgactgtccgggggcagaccgcaaaagtttttgcctgcttctgggcaaaaagttttgcgggatgtgcatttcacccctggactggcggagacggttgtggctgtctatgcccccggcgacaggggcttaaactggtgaatggcaccggacacaacaacgggcagggacccgccgcaacgacgtgaagcgcggtttgcgaaacggcgttgcgccttacatcccggcgccttttgccgggatgccgggagcgcaggtcacggcgcgacagaggcggcgcgaccggaacggtcgctgcaagccaggggttttcacgcatcggaccgtagcgacagcccgaaaccgcgccagcgggttcggcggagactggcaggtgcgccagcagctgcctgtcggagtcgggcctgacgaccggatgccgccaggcagccgggagccgaaggtacgcccggcagacaggacaacactgcagtagtcagaatcagagcaggaagaaaaagagaataatcacagggaaggtgatgtgatttctgacaggccataaccagagaaggcgggggaaaagaacgggcgccgcaggcgcgcgttcatccccgttcctgaagggaaacgaccttcctgaagtcaggttgttcatgcccgtacaacatcagcaaggagaaaggggctaccggcgaaccagcagcccctttataaaggcgcttcagtagtcagaccagcatcagtcctgaaaaggcgggcctgcgcccgcctccaggttgctacttaccggattcgtaagccatgaaagccgccacctccctgtgtccgtctctgtaacgaatctcgcacagcgattttcgtgtcagataagtgaatatcaacagtgtgagacacacgatcaacacacaccagacaagggaacttcgtggtagtttcatggccttcttctccttgcgcaaagcgcggtaagaggctatcctgatgtggactagacatagggatgcctcgtggtggttaatgaaaattaacttactacggggctatcttctttctgccacacaacacggcaacaaaccaccttcacgtcatgaggcagaaagcctcaagcgccgggcacatcatagcccatatacctgcacgctgaccacactcactttccctgaaaataatccgctcattcagaccgttcacgggaaatccgtgtgattgttgccgcatcacgctgcctcccggagtttgttcggcaccatccaccgctccatctcacgaacagcatgaccgaccacccttgccgccataccagcctctgcgatgacctgccagaatggcatatcctgccactgccagcgttccattccggccagtgacggaaaacgctcgccccactgccagaacagggtttccaggtcatgctctgccacccggtcactccagcgtgcataccagcttccgactgtttccggcgaatggaatgccagttcgatttcagcctgagcgaccagcgcctggtacgcacgggctttctgctctgcctccctcgcctgctggcggctgtacttctgcatgaccagcgcggatttatggcgactgcgttccctgacacgaaaccgcaccgacgggaacaagcgttctgctgcatcgcctggaaggggaaggaggcaaacctcgccctgtgtttcaatcagcacatcgatggcatacagccactgaagacggttgccgtgcagttcttctgcggtcagggacggcatgaaaaatcgcaggtcagacagcgatatcctgcggcttccgttcagacagcggaaaaacgcccccgcatacggatattcagccagcgtgttaccctgatggcggcgtttttcggtctccgccaccgcccgccatgcggcctgttgctctgtgctgagaggaaccagtgcctgtgaacgtgcgctcatgcctgacttccttccgccatcaggatatggatgatgtcactgacgctgtacccctgcgcgtccagtgacgccgccagtgccagtgaatggctgacagcttccggtgcaaaccgccgctgtacctgaaccaccgcaaacggctcaccaccggcattcaccagaaccagcacccagacctgagagacatcgcccggtgaacataacgccaggtgaaaacgttcgtgggcaggcgtgaaacggacctggacggggaaaaagccgccaaactcgctgccgtactcgccgttcatcgtccagttatccggcgcatccagctcccgcatcacggcgtgagtcagctcacggcgttcatcacttccggcagcccggatatcttcatattcctgtgtgttcatggtctgtaatacattcagggtcagttcagttttcatcacggtatctcccttgctgccgccccgtctccggcggggcggtgtggttgttcatcaggcggcatcagccaggttatcggtgcgttcagtggcatccgtctgtgggcgtggtgcacacatccagcctggaacccagcggttgtctttcatatggtgctctgcatgttcagccgcatcacctttcttcatcttctccgcgtcccgtgcggcaccggacagccctgccttattcagggctgcgataatctgcggcggacgttcgcgcgtcgggcacctctcttacttaagaatcccgaaatccctgatgacatggttcatccagtacccggcaccgtcctcaaaattccaggtacgccagaccattgcaccgttattacggacaaccaggcgaaactgttcgccgtggtcatcctcgataaagacgttccggtactgtgcggcaacagcttcggcatgtttgcgggtaaaaggaccgtcaggcagcccctgaagtattctgaaatattcgctcatcctcatctctccggtgatgccccgcacagaacggggcggggcggggcggttatcacagtcagcccgatcagaacgggatatcgtctgaaaacccgtaatcgtcaccctccggcggttgcggctgaggctccggctgcgcggccttacgtccacggcctttcgtttttgcgccaccttttttcgtaccggcctccgcctgtggttccggctgaggctgaccgctgaactgttgcccctcttccggctgagtctgagcacctgcggcacgtcccagcatctgcatggtgcccgtggtcttaacaagaatttcggtgacgtaacgggtaataccgttatcctcccagctacgggtgcgaagctgaccttcgatgtagacctgcgcgcccttgcgcagatattcgccagccacttccgcgagcttgccgaacagcaccacgcgatgccattctgtctgctcccgcatctcccctgtctgtttgtcacgccaggtttccgacgtggcgacctgcaggtttgccactgcgcccccgttggggatgtaacggacttccggatcttttcccaggcgtcccacgagaatgaccttgttaatgccacgaactgccatgatgtgcctccttctgttgatgttttcagcccccatcatcttttgagtttcgggagccgcggttttgacgttttgcggggacctgccttttgttccctccggtattcaccatcacctttttgagaaccttcgccgcaaggggcatcactgccacggccggaaacgactgtccgggggcagaccgcaaaagtttttgcctgcttctgggcaaaaagttttgcgggatgtacatttcacccctggactggcggagacggttgtggctgtatcagcccccggcgacaggggcttaaacaggtgaatggcaccggacagaacaacgggcagggacccgccgcaacgacgtgaagcgcggtttgcgaaacggcgttgccccttgcatcaacgccagcccggctaagatggaggcacggcacagcccgcacacgactcgacgtccacataactgaacccccacaaggccatgctccgcatggccgggtaaagcggagccggcggcgacttcctttgtgctctgtccggggaccggcaggaaaaaggcggggattttccccgccatcgtcatcatttccggatttttgccagggcatccgccatgctgcggatttcatcccgtgcctccaagaaaactcccgcagaatatagcggtcaaacaagcggcggggcggttcacccggtatacgcagcacatcacagaccaagccactggccaagatatgttccacacactcactggcaggcattaatggaatcgtagtctcacgctgataatcagatcgacaatacaagtgagaccgtggtcccagactgattagcagaccaacaatacgagtgggaccgcggtcccagactgattagcagactaacaatatgagtgggaccgtggtcccagaccgataatcagaccaacaatatgagtgggaccgtggtcccagactgattagcagaccgacaatatgagtgggaccgtggtcccagactgattagcagaccgacaatatgagtgggaccgtggtcccagactgattagcagaccaacaatacgagtgggaccgcggtcccagactgattagcagactaacaatatgagtgggcccgtggtcccagaccgataatcagaccgacaatatgagtgggaccgtggtcccagactgattatcagaccgacaatatgagtgggaccgtggtcccagactgattatcagaccgacaatacaactgggaccgtggtcccagatataatattcaggccagttagttatgttttctagcctgtgctaaaggaaattaagtaaagacagataaatgtagactaaaacgtgccgcatcaaagtgccgctttttcaagttccttaagaatggcttcaattctctctatacactcagttggaatacgagatctatctaggtttaacaccattttatcgcccttgtacaacactgtcgctccaggagcaaactgatgtcgtgagcttaagctagttcttgaggcagatgacgttttaagcacagacgttaaaagagtgatgacttcttcagcttcaaatatcactcctgcttttttctgctcgtgaaggttagatgcctgctgcttaagtaattcttctttatctgtaaaagctctttgaagtgcatcacctgaccgggcagatagttcaccgggatgagaaaaaagagcaacaactgatttaggcagtttagctgtgttgatacagcgggtaataatcttacgtgaaatattttctgcatcagccagcgcagaaatatttccagcaaattcattctgcaatcggcttgcataacgctgaccacgttcataagcacttgttggcctataatcgttacccaatctggacaatgcagccatctgctcatcatccagctcaccaaccagaactcgataatcactttctgtaagtgcagcagctttacgacggcggctcccatcggcaatttctatgaccccagacactcttcgaccgaatgctggtgtctgctgacctgtcagtagaaaagaagggataagatcatccagggcgtcttcggtaagcagttcctggtcacgttcattacctgaccatacccgagaggtcttctcaacactatctccccggagcacctcaagggtaaatttcacgtccctaccacatacaggcaaagtaatagcattgccacgagccatagctcctacgcgcgcaattaacgaatccaccatcggtgcagctggtgccgatgacggagtatcttcaagcggttgagtcttaatcgtatgttttggaatgacaggcgcacgcttcattatctaatctcccagcgtggtttaatcaaacgatcgaaaatttcattgcagacaggttcccaaatagaaagggcatttctccaggcacctgttgaagagcgttgatcaatagcctgttcaaaaacagttctcatccggatctgacctttaccaacttcatccgtttcacgcacaacattttttaggaccatgcttccccaggcgtcccgaatttgctcctccatccacggggactgagaaccattactattactgtatttggtaagcaaaatacgtacatcaggctcgaatcctttaagatctacgtttttgagcagatcacgaagcatatcgaaaaactgcagagcggaagtgtagtcgaacaactcagcaggcgtggggacaatcaacacatcagcagcacatacaacattaatcgtgccgatacctaggttaggcgcgctgtcaatgacaatgacatcatagtcatgagcgacggtttcaatggccagtcggagcatcaggtgtggatcggtgggcaatttaccttcatcaaatttgcccattagctcagtttcaatgcggtgcagagccaaacaggaaggaataatgtcaagcccaggccagcaagtaggctttattgcataagtgacatcgtccttttccccaagatagaagggaaggagagtatcctctgcatgaatatgaagatctggcacccagccgtgatacatcgatgctgttccctgggggtcgttaccttccacgagcagaacacgtaatcccttcagagccagatcctgagcaagatgaacagaaacagaggttttgtaaacgccccctttatgagcagcaactccaatcaccggcggaaatacgtcttcagcacgtcgtagtctcgtaccaaacacgtcacgcatatgattaatttgttcaattgtataaccaacacgttgctcaacccgtcctcgtatttccatatccgggtgcggtagccggcctgctttctcggcatccctgatagcctgagaagatactccaactaaatccgctgcttcacctattctccagcgtcgggtgattttcctcgcttccgggctgtcatcattaaactgtgcaatggcgatagccttcgtcatttcatgaccagcgtttatgcactggttaagtgtttccatgagtctcattctgaacatcctttaatcattgctttgcatattttcatttaatcttgcattttattgcaaagcaacaacaaaattgcaaagtagtcaaaaaaccgcaaagttgtttaaaatgagagcaacacatcaaaaaaggaaacaggaagagcacatacctcagtcaataattatcactagtactcgtagccgtcgtgtaaccgagcgtagcgagctaactggcgaggacgcgcgctaactgttcacactcagtatccctagcaccaaaataactatctgatggtcaaggcggtaatgactccaacttattgatagtgttttatgttcagataatgcccgatgactttgtcatgcagctccaccgattttgaaaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggcagttacccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacctttattcattataaaatgccccgtaatatttatctcacaatgattatgattagtggatacgcccctacatttccagacagctgttatcacttaatctattactggcccgcagccgtagatattctagccacggtggtaacgagtatccggaagagattccacttacactctttcaatcctattgcaaaagctgatttactcaacaggattaaaagagggagtttgatacaatttatttcactttttgttagaatactaattcttttcttgtatttttttgaggaaatgagcacggaacacgacataatctgtagtcagtaaagagtctatcctccctgactacagattatgtcgtgtattatatgatatcaacttggctgtcagcggccatgttgatatcagcgtacctttgttgtaaaggaataccggtcagaacagcacagagtagttcagaccaaaggttcggcccctgcctttgtagtcgtacagtgacgctgggccgtaacccgggctgtagtacagtggtgcacgctgcccccagacagtggtgtagtcacggtcgaagaggttttcaatgctgaagctgagtgtacccaccggaagctgataactgccgagcagatccacggtggtatagccatcgaccttgtagccctgcgcgtcgctcacgtcaaaggaggtggtgctctgcacgcgcagactccacgggtccggtgcccagccaatgtaggctgtcgcttttgatgggcttgctgtcttcacatcgtatttctgccaggtaccgttcactttcgactcagttttcagcacgttgaagttcactccggtactccagtcagtatcaggaatcaggtagtccaccgcaccttccacgccgtaaatacggcgtttgtcgtccaccacgctgatggtcagatctttattcgccaccacgctcttatcagaaatcgaatagtaggccgcgatttgggtacgcagattattgccagtaaagcgccagcccagctcataagaatcgactttcacgccttccagcttgctgtcgctgacgttcacactctttgttagaggaagatggccgttcactgcagcaccatagatgccgcgaccatagtatttacccgggtccggcagctccacgccctgggagaagttgagccatgcctgctggcgttcggtgatgtgcatcagcagaccggcgttgaacaggaagttgtcgtaatcgactgagccgccaggaatggcgtcggcggatgtagccttcccggcggcaatctgccgttgctgcgcgtagccgatgaaatcatcaatcttgttttcagtgtactgatagcgtacgccaccgttgagggtaaagagattattgatgtcatagcctgattgcaggaagaccgccaggttggtgatgtcatacgacggatagcgcccggtggtgtaaatcttcttgttgttcagccctccggaagcgcttgcctgagccaggtcgaagaacatctggttggaggtaaagcgctcatgatcagcatccagcccccaggtgatttgccagccgtccatcggtttgctgttcagagtcagtttcatgccgtactggtcggtgtcctgctgtgacgaagagaaagccgtcacctgtttgttcgcatttaccgtcgggaacgggtagaatcgcaacgactcatcgcggtagtaaacctgaccgaccagctcctgtcccagaaaagcgctgtcagagtactgcaggctgatcaaatgccgctcagtgccgggaatacggtcggaattcagcccgttactgacgaatggcgtgctggtccctctgatggcagagaagcctttcccgagattaagcccgtaatcgtcgtcgccctggcttttatagtactgtgtgatcaactgaagctgccgggattcatcgatgttcagcgtaccagttcccatgatgtccagccgatcggagtactgcaggccggtctgggtgttatcaagcaaggtggcatcgccgttaccgtcaaaccagccgccaaatttctgatatgccacggaaagacgtccggagatatgctcatttccgccggagacagctccggcaatgcgttcatcgtgatctttactgctgctaaagccacttttggtgccagcctcaaactccatcatggtttccggctggccttttttggtcacgatgttgatcaggccaccggtactgccgccgccgtacagggacgtcgcaccggagatcacttcaatatggtcgatattaaaaggatctatagagtccagttgtcggctgtcggtacgtgaagagttgagacgcacgccgtcaaccagcacgaccagcgggcggccacgcacattcataccgtagttggtgcggctccggctgctgacgtcaaggccagggatcagctgagccagtgcgtctttaagctctttgccgccctgaatctgctgttccagttcggcgttttcgataacccaggtggtttgcgccatctccgctacggtgcgattgctgcggttggcagacaccacgaacgtttcatcatcggtttgttgagcgactgctggcgccatcatggtaagaagcagtgggttgagagcccaaagcgtatactttttgcttatcatcattattcctccatacgggcggaaaaaatcagcccaaagatgctgtgtagcgagttaaagaagcagcctccggctgcgttttccctaggtgccgctgcgccactgaatcagggcgggcggcatactgagatcgaataaatcacgtcccattacgcgattaagaatacgtgcagatctccatgccatcaggctgagctggggttcggcgatgccatgggtttgcatactggcgttgaccacgaagatgttgttctctttcgggccactccattccagagtgaagtcatcacgcactttaaaggtgttcttatcgtgcatggtgatcaggggcatcagtgagggaagtatttgtggcaacgcagaacggtaacctgtggcgaaaatcaccacatcactttccaggctctccctgccctgatccagatgatgctccatcagcaacttccagccaggaccactactttccagagttgttaccgagcggctgggtagcagacggatatttcttggttttctcagaacttcaaaacggtggtacaactcacgataaatggtcagtaaagaatcggcagtgatgccatccgatgtcattttctgctcatccagtaactgatggcgaatatcttcctccagtccggagaagcctgaaatatattcaggtgtaaaatactcatcagcaaaagcagcctcatccagtgcgttaaaattattacgccgggagacccagtttatttccgccgcttctccccattccccgcgtaatgcattaaggaacaggtctgcaccactctgtcctccaccaaccacggttatccgttttccactaaggtccggccgacgaagattcatttcactggcatggaaacaggattgtgtcatatgcttcacacagggtggtaaataaggttgttttcctgtaccaaggcagatattgcgggcaaaatattctccctggctggtttgcaccagaaacaatcgacgttttttatcgaaatcaatgttttcaacggtatgactgaaatacaggttattcatatcttcagcagcccagcggaggtagtcagaaaactcttcacgggatactgtacgtagtctgcttgtaaggaagcgatagaactttttgtgcttcaccagatagttaacaaaactgtagggatttgtaggtgcaacagcactgaccagatctttcagaaagacggtctgcatatgacaatccggtaccagcatacccggatgccaggaaaaatgaggatgttcatcaaagaagagacagtccagttcttcgatctgatgtgacaacgcagcaatgctgagattaaatggccctgtccctacaccaataaaatcgacactttttttcatgattcatattcctgagttaccagccacagcggattttgcagatcctcaaggtaattcggcagcatgcggctgccgccatccagatcaggccaggtcagttttaccgggttcagcaccacgcgaatgatttgtggcctgaaaagtgagaaaagcgcaaaacgctctgacatttgtggatgttttttcatgtaatcactcaacactgctgccagcagttgataaaatcgcctttcaggtacgccaagacgaaccatcagtggcgaaataaaacgcagtactgtcacgaagtgacccgtctgcaaatcatggattaagtagtccgcactcaggcgggatgtaacatcacgaacctcctgaggcaaagagtccatttcggggaactcttctttcaccagtcgcatatcgccctggaagtctttcagcagaacacgctgtggaaccccctctttcatggcgagagttatattttgtccatgtgcaataagcgcgacaccgtagcggcaaagcaggtgatacagaggaaccacgaccacccggaacagttgcgtaagccaggtttcagcgtccagcccggagcggtctatatatgcgcctgccagcggctgattgttttcgtcgcactccatcagtgttgccatcagaaccggactttcatccggtttcagccagcggcacggattctcccgccagataacaccaagcatttcctggtagcgatagggagcccgggcaagcgcggcatagccttcatgggagacatagcctgcagccggttcaccaagtatcactgcgccgctttgcactagggtggcgtcggtcgcaaaaacctgttgtagccagcgtgaagccagtggtccggcagcgatgtatctgccagggatcccccggtagcatgaggtgttgtagatggtcagcggcagcttgatatccagcccgccccggcgactggcgttggtcagggtacgcagcgactgctgggcgagccactggtcgccaaactcgccgagagacaccatcctgccttcggcaaaatcagcgatgaagtcggtagcgattttttgctgccactgccacggatgtaccggcagcggcagccagttatgatccagtccgttttcctgccagacctgactgaagcgggcaaactcctgcggatccattgcggccgtcaacaactgatgaatatccatctcgttatcacagcgccagatcatatgttcacgttttaccgccagccagtgcagtctgaaggtgttggcatactctggcgcatatcgttccagcgcctctttaccccagccacggcgacctttattaaaaacgaatttaggatgaccgctcagcaggcattgcaggcggtcggcattaagattaatcaggtcactggcgctcagcccgcgacgggctttcagtagttgcaggtcgcccagcagcgtggcatacaaatcctgcatatgctcagcaacggttgcatcgctcattgacagtacctgctttagctgcatcagcagcgtctgagccagtactggctcgtccgcgcagcgcagagtttgagcatcaatccagagccagccccagataccacgttcagcgatgaagcgccattgtgctcccggcaggttaatgcagtagcggtcatcgccctgagattccgcgtggaaaacctgctcatactccagctcagacaacatttttgccaccaggcggcggttgaccaaatcccagtctttgtgattcattacagaccgacctccgtgaagaagtgatgacgatccgccatcaccatgcgcgagcgtttatgtgggaagtcgaactctttaattgtccggtatcccgcaggctcaagatggcggaacaggcgctggttatcggtgcgtggctccagtactgtgcgctgcgtacgtggctcattcagcagcaggtaatgtgtcaccccgcgcagccagctttgcacatagtgggctccgcgccattgctgttcaccaaccagcagatgcaggccacggtcaaagggctgccacgaatagtggcgaccaatgcggtcttccgccgcccagtagatttcgaaataactgaacggccgatcgtcgaaacaaccaatcagcgggaatgcatgtttactggtgagctggcgttccagataggcgatctgtacctccagtgagccactttgctcccagaaatactcaacgcgcggatcgttcatccagcgagtgaaacgttctgcatcagaaacgggatcggcaatgcggaaactcagcatcctgcgaatgcgtggatcgtaacgacgataaacttcaccctgcggacggggggggcgctgcgggaaataaatctcacgctctgcatcaaaaaccattttaccggaggcccggtttgccggagaactcagccataacggtaactgccagaaagcggaacgatggataatgtcgctttgtacctgtccgaaaagcgtcagcgcctgtggctcctcacaccattcggaccagggaagaaccactgctgaaagttgtggtgcggcgataaatatctgatccagcgcgtcgcacagccaccctgttggcagctccccgggccagtgcaacaccgcgctattgtccagcccccagccaagattcagaggcttgtcgagtttttcacagcgcagtccatatccgctgtgaacaatgtttgccccagacattcagacctcctgagcctgcagcgggttagcgaaatcaaaatagatcaccgaagggtcaacgatggtgttctcgttatgatcgttcagataacagaaaaagttaccttttacgttccagtacggactttccagaacatagttgagacaggttttatgagtcacctggtcacgcacttcagccagcaatgttcgtacacgagccatcagattcgcttcgctgtccagcccggcagcacccagcgcagcagttacggcaaaagtggagttaaccagcaggtaataagggaaatagcgcagcaactgctcacgggtgaaaatattttccgcctgcgcctcgtcaatggtatcgagccactctgtcgcgtgaggcataaacgcgctgccctggcagtcacggtaaataaatccgaccggcagatccccaagcatctgcacaagaatattttgctgatgcgccagcagcaccaggccgtaatccgcttcagccgtaaacagaggcttcagcacctgctgacagtaagcatcgacccatgcgtgggcggcctgttgcacagtgataccgaggcgatcgctcaggcgttttaccgccgaaaccagcagcgaatcaccgccatccggtgcggcctgagtcagggaaaccagaacgttggtctggctttgcggctgctccagcagcaggttttcacgcagggaaaacaaactttcctgcatgatgttgccgttaagatcgcgcagcccggcccagccgtcctcctgcattacccggaaagtagggaagcgggcctgtagcatctgccagccgtcggtttgcgccagacgtgccaggcgcattcctcgctccacctctttcacagacagagtacggacggagttggtcagccgaacgctcagggagaacttgatcatatcgcggctggtagcacagtagagggagcgggaagaggtggtcggcagccacgacgtgccggcctcgcctaagtctctgataagtcctttagcaaaaagtgcctggcaccacacttgctgcaaaagatattctccctgccacgggtgcagcgggaacagccattgattgtcacttaattcgttgagtaactggggcgcattttctgcggcaaatcgcgtcaaccgctgttgaaggttaagatgcagactttcaccagcgatttgcgttttatccaccgaaaaccaccgcagcgggaagtgaggtgccatgtcaggcaggtatcgttcagcttcctggcggttaaacggttcatgagacttaggtgcagggtggaaagcgtgtcctgtcagcagtgcctgctcagcctcgccaaaattcaacgctttttcacgcaggatggcccagtcatggcgggcatcaattgtctgttgcgtatgggcgtagctttccagcacgcgttggtggaaagtttcgcagaggggaactggcagaaaaagttgatgccggagcttatcaataataagccgggagagagtggtaaagtcgacaggataactgcctgatgccgtgaccagatgagcaggaaatgcaaagcggtgatgctgggttggggagaaataagccactctgaaatggagcgacttctgttcgtccagcgggataatcaattcgtctggcgggtattccgccagttgccagtctttggtttcacgaatcaaggcattcaggaagcactgcgcagccacatctgtggcggatttttcagagggcaggatcataatcaaacagctcctacacagtaaaataataacaattatgtcaataatgattctcattatcaatgagaaatgttattgtcagaaaaattaattttacaaataaacacaattaatacatggtgttgaaacattacaatgtcatctaagatcgaagatacgccccaaaaaaccctgtcctgctggccactggcgttcagtgccggtcttctcggtatcggacagaacggtctgctggttgtactccctgttctggtcatacagacaaatctgagtctgtctgtatgggctgccctgctgatgctgggctcaatgctgtttctgccatcttccccatggtggggaaagcaaatttcccttactggcagtaagactgtggtgctgtgggctctgggaggatatggcgtaagctttaccctgcttgggctgggaagtgtgctgatggctaccggtgccgtaacaacagcggtggggttgggaatattaatcatcgcccggatcgtctacggtctgaccgtgtcagcaatggtgccagcctgtcaggtctgggcattgcagagagcgggagaagggaatcgcatggccgctctggcaaccatcagctccggcctgagctgcggcaggctattcgggccgctgtgcgcggcggcaatgttggtcattcaccctctggcgccagtgtggatgctgatggcagctccggtgtgggctgtggtgatgcttctgcggttgcccggcacaccaccacagcccacaccggagcgcaagagcgtcagcctgaagcgggattgcctgccttatctgctttgcgcaatgttactggctgcggcaatgagcatgatgcagcttggactttcgccagcccttactcgccagttcgatactgataccaccactattagccaacaggtggcgtggttgttggggctgtccgcaatagctgcgcttatcgcgcagttcgtggtactccgtccacagcgcctgactccagtggctctgctcctgagtgccggggtgttgatgagtagtggtctggctatcatgctcactgaacagctatggttgttttacctgggctgtgcagtgctgtcatttggggccgctctggcaacacccgcttatcaacttttactgaatgataagctggccgacggcgcaggcgcgggctggcttgcggccagtcacacacttggctatgggctgtgcgcattgttggtgccactggcgtcgaaaacaggtgtctcaatcgcgctgattgtgacggcattatttgccgctgtattatttaccatagtgtctgcatgtatttggcattaccgtacaatcaagtaaggctatgatgttgtactcacagccggcataacgtcctgtgcaatttcgctgtcgaaaccgcctgtgctgaccgcccagtgcactcacctgaagcacatcgcagcatgccaccaggatgccagatcgtcttctgaaatgggtggatacattggcaacgttaaccgcaacaatttttcagagagaattttggggggtgagggccagtgatgtaaaaaatacgccaagaggttaattcattgtttatactgatatgtcaggtaatgcgtttgcagtcattacagtcgtcatggagactgtaatgactgcaaacgcataccatggagcgaaattctatcctaagcgtcacattagttgataaacctgtaacgttaatgaattctcatgcgaaaagtatggttatgataaaaaagcctaaagctgtcatgatgagggagccaatgacgtgaacaagtacagacgtcaaagcccatatataatttcccgcctggagtaatgcgaatacttcaacagaaaatgtagagaaagtactcagacctccacaaagtcccgttgttatcattaatttccaaaaaggatcaaggtgtggttgtcgtaaaaagtacgccaacgcagtaccaataattaacccggcaaggaggtttaccaccagtgtacctggaggtagatttggaaaaagactgttaaagcgtgtggacagtaaccagcgcagagtacatccaacagaaccgccgataatgacggcaaaaagtgatttaatcatgtacccctcataaaataattttttatcgtaacaatatcagaatatgttctcatttgactgatattgttaatttatatcctttgactacatgcaagataagggaaggttctatttattgacattaataccctcgacaattgctgagcagaagcccccacgacaagtgacagaatgacaacatctacagtggggagcgttttatcgatgaacaagcaaccaagaagtaacacggcataggtgacgacaatgccagtaagcatagaaataacaaaaacgtgagctttcatcattcctccagaataaccccgacgtgtaagcctgggggattggcacaataatgtcatttaaaggcagactttccagcaaattgcgccgatgaacccagttcatcctcaattctcataagctggttatatttttcaatacgctcaccccggcatggagctcccgttttgagatgacctgcgcgcattgcaactgtcatatcggcgataaagctgtccactgtctcaccactgcggtgtgagataaaggtgccccagttgttgtcatggcaaagttgtaccgcatcaaaggtttcactcagggaaccaatctggttgagtttaatcaatgctgaattggcaaggttttcgtcaatgccgcgctgaatatactttacgttggtcacaaacagatcgtcgccaaccaattctatctgctctcccaatgcaccatgcaggattttccatccagcccagtcatcttcagctaccgttaagggcaggaagatcaactaggtgctctggggctggtgaccaatgcggttgtgctgtgaaacacactttacatgcaggaggcgttgtcacatcttcgcaaatcgggagaaactcctgaggagtagcatccggcaagattatcgctgctgatacatagccatataaatatgctggggcattatacgtcttcgttaccggaagatattcttaagggtgagttaagatcattaaacttcaatacaaacaattaattaaactcttagcgtggttttttacatcactggccctcgccccccaattttctctatgttttgccactacttccctcattttactgcgctgtttgctacccctgagaatcgacattacttgctatccagtccgcagcaactctctggcaacaaaacttatcagaatcataagtctgtgcattattcgttcaagcgcatcgccaataagcggatgttgctggccgagtgttctgctaatcggatgataacaaatcccgtggttcggcattttcctttgtgcgtgcgttgatgctggtaacggtaaacgtcatgatgaacatgaccgcgaataagacaacaattgtcggagctggcgcgctgtcgataaaaaaggacagccagacaccagtcattgagaccaatatcgatatgccagtcgccagcaggagcgcaatatgaaagcgttgtgtcaggagtaccgcaatggcaccaggcgcaatgagaagagaaatagagagaatgatacctaccgctttcagcgtcgccacaatggtcagggagaccatgcacagcagcccataatgcagccatctcgtatgcaaaccgcttgcctgcgcctgttgataatcaaagctgaacagcaaaaaatcccgccattttacgctaataacaagtgtaaccagtccggcaataatcaccgtctggattatatcgccgatggttatcccgagcatgtcgccgaaaagaatatgatcaagatgtacgtctggtttgactgcaatatacaagatcagacctgcggcaaacatgccagaaaaaacaatccccatcactgtatcttgcttgattcggctgttgtctttcaggtatcccgtcgccaccgcacagaatactccggcgacgaatgcccctgtagccagaggtaaccccagaatccaggcaagcacgatgccaggaaaaaccgcatgactcatcgcgtcacccatcagtgcccaacttttcaggaccagaaagaccgacagcagcgcacagggtatggaaactaccagtgaaatcagcagtgcgtgactcataaaggtgaactgcagtggctccagtaataatgccatcatgagcgatcctccagcgctttatgcgcacgccgacgatttgccaaaagcccatgggtgggcgcaaagacaaatgctaaaagaaacagcagtgtctgcgccaccacgatgattccacctgtcgcgccgtccagaaagtaactgacccacgcgccgaggaaactggtcacactgccaatagttacagcaatcatgagcaggcggggaaaacgatcggttaacaaccatgctgttgcaccgggagtcaccaccagacagatcaccagaaaggcaccaaccgtttgcagcgccgcaacggtcgaaaccgacagcagggtgaagaagaggatttttaatctgcccggatgcaatccaatagcacgggcgtgattctcgtcgaaaaaggtcaccataagatctttccatttgaaaaaaagcacgatgatcgagagaatgccaatgatagtgagttgcaggatatccgccggatcgattgccagaatattgcccaggacgatggtctgaatgttcacggatgtcgggttcaatgacaccataaacagccccaggccaaaaaaggacgagaagatcaggccgatgatggcatcttcttttaagcgagtgagttggttaagaaacagcatgctgcccgctgccagtccgccggaaaagaaggctcccagagaaaacggaagccccagcatatatgcccccgcaacacccgggacaatagagtgcgacagcgcatcgccaatcagcgaccagccttttagcatcagatagcatgacaggaaagcgcagagaccgccgaccatcgccgagacccacatcgcattgagcatatattcatagctgaagggttccagtagcacgttcatctttcttccctctgcaccgctgacggtcgatgtgccacaaaagggcgttcatcgtcagtaataatgctttcttccgagccgttaagtgtgacatggcgcagtacgccgctaaaagccagctccaggttctcggctgtaaaagtggtgtctgtcggcccgcttgccaggacggtgcctttgaccatgaccgtataatcgcaaaatgtcgtgacggatccgagattgtgcgttgagacaagcatcgttttgccttccgcgcgtaactccctcaacaggctgatgattttcgcttcggttttcacatcaacgccggtaaaaggttcatccagcaggatcacatcaccctgctgtgcaatcgctctcgccagaaagacgcgtttcttttgtccaccggaaagctcgccgatttgtcgatggcgaaaatccaccatatcgacacgttccagcgcatcggtgacaatctgacgatccctctttttggcgatacgcaacatacccatatgcccatagcggcccatcatcaccacatcttccaccagtacaggaaatgaccagtcaacctcttctgactgagggacgtaggcaaccaggtttttctgtagcgcctgtcgtgtgggaatacccagaacagatatcttcccgctggtcagacgcacaaatcccataatcgctttaaacaacgtcgattttccggaaccgtttaccccaaccagagcggcaatagatccgcctggtactgtgaaggatgcatcgcgcagtgctgtgtgcccgttacgccaggtgacagtaacatcattaacgacaatgcctgcagattgcatcattatttctccctttttccggctttaattccctgtaccagcgtactggtagtaaccttcagaaggtcgatatacgtaggtaccgggccgttttctgtgctcagggaatcgacatagagcacaccaccgtagtgcgcgccggtttcacgcgcaacctgacgcgctggtttatcggaaatcgtactctcgctaaagactgccgggatatgatttttcttaactatatcaacaaccttacgtacctgctgcggtgttccttgttgatcggcattaatcggccacagataaagctcttttagccccaaatcgcgtgcgagataagaaaaagccccttcactggtgaccatccatcgctgattctcagggagttccgcaatctgcttacgcaggggggcaagggtttgggtaatcttggctttataagtatcggcattacgttggtaggtttgtgcatttgccggatcgtattttataaacgcatcacgaatattatcgacgtaaatcagagcattatctggcgacatccaggcatgggggttaggtttgccctcatagggcccttcggtgatccctactggcgtcacacccgaagagacaattacttctggaaccccattgagatgctggtaaaagcgttggaaccacaattccagattcataccattggcgagaatcagttgtgccccctgcgcacgtttaatatcgccaggggtaggctgatactcatgaatttctgcaccaggcttggttatggatgagacttctgcagcatctccagccacgtttttggccatatctgcgatgatggtaaaagttgtaatgaccttgaatttttcagctgcactcgcctgaaatgctatcgagcaggtgaggactaggcaccccaagagcatggttactttttttattgagagcatgatttagtacctatagtgagcaaaattagaataaacatagcaactactatgttttatagcagaagctataaaatgtaaataattatcattagcgattataagatcccaacacagggattaggttatttcctgctgacgatgtggtattgaggataaaagttgggcagcgctttggctttaaaatgaacttgttcaggacgattggtaatggcaaagcaaaaataatgtaggttattggcaagttctctaggacgcaggagagcatgaaaaacccaggggcagtcactaaatgaagggatcggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggtaactgcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacctgctttttcggttgaaaagaaattgattgaattactggaaaaacaatctggtctccatacgtctgtggattgcagtactccccttacagagcagagtctgtcgagaaatgacggtgaagaccagaccagaagaaacgctgaaaatatgttcggagatgactgaactaacccagcctggccaggggaatatctcgccatttagttgtataacactgactcaacatttcccgtttcatttgccattcaggggcggttcctcgccccgcaaaccacactttcccttttcccgactggttcagttcatcgagtgttttcattaacttttcactgtttttacggggctggatttcatcaaataatcccggctgcgctatacccgatggtgtgaaatcagccagcatgactcctgccttcatatagcggtacccttcacgccagacatggtttaaggctctgcatgcggcggcaataatgtcccggctgtcctgtgtgggcaatgaaagcttttccacagcggcattgctgtaacagggttcagggggatgtccgtacaaatgtcgtcacctgccggcaatactgacgctccccacgtagtttctctgcggcccgctctgcatactgaacaacagcctggtgcatggcatctttgtctgtgattcgttcaccaaaactgcgactatgtcaacgccacgatgtttgaccgttatttgccattttcatgcccgctaaatactttcgttactgatgctgtccgctttgcaccatttcatcatcactgattggcgttgtgtttttggtcagcactccagctttacgtttctcttttaaccggtaactctctccttgttctttttgacggacgcattttacgtttgggctggatgtagtaacgcagcatggaacgtccgcccgtataacccattgccttaatctccgcgaagataacctcgctattccagacattctctgccaggcgcatgtcgatgtaatccataaacggtttcagcttaaccattttgtggcgggtctttctggctggcggttcagggtatttgaggtagcgtctgaccgtccgttcagagcaaccaatctgagccgcaatatcgacaatgtacgcgccctgctggcgcatttgctttatcatgtaaaagtcctctctgctcagcatgttgatgtcctttctggtgtgagaaccttaaggaaacaacatgttgggtggagcggacaatccaaatggtgaattaccgtcttatatcactggcgctgacacctgtagtttgtatacacaatccctggcattgaaacacacatatgaagtgtacttcggatatgaccgtacgctcaggaagtacggcagattccgtcaaaaattacctgtaaagacagacatcagaaaaaacagtgctcgccgcagaccggtgaccgggcgaagcccgcgaaccggcgaggaagcgcaatgtatctgtagttacagggagcaggtacgccctgaagccctttcataaaacggttgtgtgcgctgtccttctggaatgatttatccacatatccggaggcccgatccagtaaacagatccatgaatgatcaacaaaggatccattaaagatccccatatcgctgcaaaccttgtcactcatgggccgggaccacgatcacataagctgtggcatgttactgataaactgtaacatgctaatgataagctgtattcagtaatccatatactgaagtaagttaatgacataaactatggtcagtatgccagactcagttgttaaatacaggctgcaggtttttcttcagtcagttagcggagttctgacacacgattttctgtttattcttttactgtccacaggctggaggctttctggaaaacgaaaattcagacatcaaaaaactgttcggcgaggtggataagtcctccggtgagctggtgacactgacaccaaacaataacaacaccgtacaacctgtggcgctgatgcgtctgggcgtttttgtaccgacccttaaatcactgaagaacagtaaaaaaaatacactgtcacgtactgatgccacggaagagctgacacgtctttccctggcccgtgctgagggattcgataaggttgagatcaccggcccccgcctggatatggataatgatttcaagacctgggtggggatcattcattcctttgcccgccataacgtgattggtgacaaagttgaactgccttttgttgagtttgcaaaactgtgtggtataccttcaagccagtcatcccgcaggctgcgtgagcgcatcagcccttccctgaagcgcattgccggtaccgtgatttcgttttcccgtaccgatgagaagcacacccgggaatatatcacccatctggtacagtcagcttactacgatactgagcgggatattgtgcagttacaggccgatccccgcctgtttgaactgtaccagtttgacagaaaagtccttctccagcttaaggcgattaatgccctgaagcgacgggagtccgcccaggcactctacacctttatagagagcctgccccgggatccggcaccgatatcgctggcgcggctgcgtgcacgcctcaatctgaagtctcctgtattttcccagaaccagacggtcagacgggcaatggagcagttgcgcgagattggatatcttgattacacggagatccagcgggggcggacaaaattcttctgtattcactaccggcgtccccggttaaaagcgccgaatgacgagagtaaggaaaatccgttgcaaccttcacctgcggaaaaagtcagtccggagatggcggagaagcttgccctgcttgagaaactgggcatcacgctggatgacctggaaaaactcttcaaatcccgctgaacataaactgtagtcagtgaagagcgctcctttactgactacagcttatattaaccggtgcagtgagtggtctgctcactgcagtttatattctgtttcctgcagtgctgtctgtagctgagctgccatctgcctgtcccttacgtaagtgatcccgtaacctgatcctgaggcattgctcccttcataaaacatgacttactcactacagcttatatacatgctccagcttatgttatgtctgttctgctgaccacagcttatataaggaagcgcgtatgccgtcacctcaggggaggcagtgtacgcaggatctccgcagcatcccgcccgtcacctgtgaaaggcactgccagcgtggcagccatatccagtgcaaacactctggtataaacctccatcgaacgtggatccctgtgaccaaccagtgcctggatgactttccggggctggcggtgatagagcatgtgcatgatatagctgtgccggaaggtgtgtggtgtgaccggaatcgaaaagtgtactccgtcagcttcggcccgtctgacagcctgcttcagccagttgcgcatggtttcgtcggtcacggcccataatggttcacgacgacggggccgggtggtgatcatccagctttccatctgcctgacatagcttatatctgtcagcggaaccaggcgcacttcatcttttggcgggcgtccgcgtcgcgcacgcactttttcggacaggatccgcacaaacggtcttactccattcaggtcaaatgattccggcgtcagcatccgggcttcgccaatacgcattccggtattccagagggtggcgaacagcatatggtgacgctgatccggcatatagaagagaagggcactcacttccggggccagcaggtaggccggtgtggcccccgtggaagtggccattcttctcagggagagcgctgttgcaaaatccacccccggcgcaatgggtaacagggagacgcgttcaggtgaattctgcaggggaatgacattgttcatgagttatttttcacgtagcacacgcactgtttatggatacaggtattctgacctgaacgcccggatccttccacgacaataaatcgcagcgcgcggatccttttgtttgattatcacgcattattgcgggtaaaaccaaatacataaaggcgcattttgccgctgggatcccgtcttaagccagtcatggtgcgggctgagggctggtttaccgtttttgggagaattggggtaatgccaggacaggcagaaacggaaaaaaaatcctgcagacgcccttaaaagacaactgaaaatttctgactccatattcctctggtttcagactgcttaatcacatcatcaacctgatacataataaacagattctgttcacagaatgaacaattaagtaaatggttagattatgcgaaaaagtacagatatttctcaccatcgttattgaataatatgtcttcacacaaacaatattaatattttgagtgtaagccctgactcaaagaaagaaatgtctgttttcggaatcaaaaatatatagttatcaaaatacattaatttcaaatttgaaattaatgtaaaacgatttagtaaatgttatgacattataaatctactcctccatgtttagaggagtatttgcagactggacgctcttatatagttacttacctttatacatattctaacataagcatttaccaaccttacagggaacagaccatagtagtttatatttccattcaggtctgaatgcaccgatagaatatatcatctttttattattatcctgacaataccttcgtgctttatctatattcagccacataaggacacttcccggacttagtttcatacaattctcatcattcagaactgcaccatttggaacgtcataataaacattacatgagctttcagatttcagtactatatcaatagcacacggtttattatcccagaatagcacatggccaaacataagttctcgtaagtgagaaataaacattaacagattatcatattcgtagcaaggtagggtacctccaaaccgggaatgaaaaagtgatatataagagctggatatttctttatcggataactggcttatacacttaatctctccgccattcctgataaatctctggatttcatttcggcgagttttttcaaatttaggtgaaaaagattcctttataatacaattttgtttttttctggctattttccatgttgcattgataatattttgtttatttataattgataatttattagttttatccggaaaaaaacatcttgcatgtggggaaaaaggaattaatacctcatcagatgaaagaggataagaccttcttgccattatcccaatttgttttccattgcacagaaaatatgagcctttgatatcaccatctttttcatagtgaaaaaaatcaaacttccagtcatgatgtatcataaagtatgaaacaacatccggatgcatgtttacactaccaccgtatttttcaaaagtttttttgtacgtttcgaatgtagatggtttccaggccgtaataatgcgctttatatttttaattgacaaagtcatatcctcggcgtttttatatgttatgagtgcaacaccaacaataattctgattatgataaatattatttaaaatcaacttccatttgttgttgaattgaaaggtgttttgtcgtttctatacagtatttaatataatcagtaaactttggcggtttaggcattccgatactcagtagcttgtcgttactgaaacaaacattgagcatactgaacgctccatataaacgaatggcttttaacataaggcgttcgttacagggaccaaaaatatttttaaaatcatgacggctcattgccagtatgtcataactgactttagtatatctgtctccaacaggatcacagttcaaagcacgggctacagcttcgtcaatagcagagaacgtcacactactttctttacctgctgatatatgaacaatctcaccattaattaacgaactttcaagcaacattagcaatgcatcagcacaataatctacagggataacatctattttatcatccagagagcacataaatttttgcaacattaaccccattctgaatacccagaaaatactggttgaaggtaagcaccctaaacgactgtggccaacaataattgatggtcgggcaaccaacaaaggtaaatcaggacactgcttacgcatcagatattctattgttgcttttgaatgcgtatactccactaaatgttcacctgtttctgatgaagcagactcttccttaactagcgaccccgtatgaggtgtacaagacatagcagtaccaacatgaaggaagcgtttcagtcctgccacttttgccattcttcttgcaaaagcaagtgtacctgtaacattcacattccatataaaaggattattaccaaaagaagctatagccgcacagtttataacatgagtgacttcatcaagacgaggatccatgagaaaggcttccggattatttagatccccaggcaagatgttatcattagttaatgcatgcaacctttcctcacaaacattaaatttacgcatattttctttaattctttccagtcccgcttgtggagtaggtgctcgtactaaaagtagcaaatttatattattacagttatccagcagcttatccaggaccgcgccaccaagaaatcctgttgcacctgtaagtaataacaatttcatatctaacctcttttgttttcatgtaatcattataaaaataaattatcttacataaactatgattaaacaaaatcaagagagattaaatgtaaaaacaatgtttatttttatacgacaggatccttcaggttattgcgagtgtttttattggatggtcttcttattattgacgagtaatagaattataataaaattcatattattaacatcgcttatcaattatttttatgtcgataacactgtgtcgttatgcatgaagagtggcagagcagcatctttaaaaatacaagagttaacagaaacacttttatcgttgagcacatataccctccacatattataaaaaatcaaaagaccaccatcagaagtgctggccttttgtttatcatatttaaagtgtcatgaaaaagttaaaaacaggcatgcttcaggttaaaaataatacttcagaccagctgtggtcataaagttatagctttcaatgcctgcaccatttttggtgtgatcagatatattcaaatttctggagtacagagaagtatcacctttcttattagtaatacggttccatgttccttcgacataaactttagctgcaggtgtaatataataaccagcatgtagtgagacagagtaatagttctgattatttacatcgctacgataggtgatccttttctcagggttgtaatgctcatcattatccgatgccttcacccatccgctatatttgaaactccccccgaactcaaagctatcataccgataattaccggttaaccctatatagggcattttaaagtgttgtttgtagcctatagctctttcaccatctggaaatgatcccgtttcatcccggaagcctccctcactactatagatataagagcctccttttgctgtaaagctataacgattttcctggtaaccagccataagacctaactgatagtctggttgattcaggagccatcccttaatattcaggtcgaattcattagcaaaatttagacgggtattcggatgtttactctcatcagtccaggttccgggattgctcgtatccagccagtccctgtccaccatattgcctccacgaccagctaatgttgtccaccctgaggcaccgacagatactctgggtaacagatcccagttaaatgcccccttgacaattggcgcattactgtatttccagtccagctggctggctttgcgccctttttcttcaggcagataaacccgttctttggccttgccacttaaagtgccaaaatcaatctccgtactgattttctccggtgtaaaagaaagcccagtatcagaagccagtgcagcaaacgctacaggagctgagagtgcagtagcaagaatctttaagtacatagacaagttaactccattccactcgtttgtttttgataaatatttttttatttactcgtagtagtcaactgaaaaatgctgttttgtttaacgtttgtttacaatgtggccgcatgtcgctttatttattcccttttatgtcagaatatcaggggagtcagtcaaacccggtatgattactgccccggtcatccatgttccaggaaatatcagggcatacagggtgctggtaactgcgaataaggccctggtcggacactttatgccgggaacgtctgtaacatggaaaccggtaccttcttcctgggtttattttgttaactcttatctaaagggagacaaaatggccaaatcctgcgattatgaagcaatcagggtttttattagcgttaatgtcggcaaatataccaataacgccattacagttaattattcagatagcgtttgtgcgacaaggcattaccattcgacgaaaataaactcaggtagttgatatcagaaatcaaccgtggctccacctacaaaaaaagttgctggtttataaatccctcctttctgattaataagaaaactaataacatatgagtataattacttgttcagataagttaattatcttcgtgattgatatttatttggtttcagtaaaaatgatgtttttattataaagtttataaataccaagtcggtggtgtttattagtgtaatggaggggagtgcgtaaatcgttacatgtaacacttccttccaataatactttataaatcaataaatgtatgattacttatttcctgaaatatcacatatattgctatatttcttaacgagatattcttattttattgatgattgcatctcttttatttaatgtaaagctttcttattaatcttctaatgtattgcaaaatggtagatgctgagtaatcaacagacttgtatgtaaagtcaagattaacgttgctaaagcacgcatcaattttctgataaaaggatgtggttgtttttttcgataaaaaaaattcttgtctaaccgctggtgaaataagattattattaccccggattaatactgagcctaacaatagaatacaaaaaaaacaaaatgctttaaataaggggagcactataagttatcctctttactttatgcttaccaactcattttgttactttataacataactcatcttgcgttgtaaacagactcgttttagttccatgcaccttttgagatcgtgaccaaataatggcgttagtgatgttcttccagtgtcatatggttaagtggttgatatggcgttcactgttatattctgacaaccagtttgccgccagttcccgcttttcatccagcgttctgaacagacaaaaaccgagtatttttgttcggtatgtccggttaaaactctcgataaaagcgttctgcgccaacttacctgattgggtaatctccagttttactacatgtttttctgcgcattacgtccgtgttggataaaaaaaatcctggccataatctatgcgaatcatagctggatagacatggttttccgcaatccttgaaccgccccgggaatcctggagactaaactccctgagaaagaggtaaacaggatgactaaaaatactcgtttttcccccgaagtccgtcagagggcgattcgtatggttctggaaagtcagggcgaatatgactcacagtgggcggcaatttgttccattgccccaaagattggctgtacaccggagactctgcgtgtctgggtacgccagcatgagcgggataccggaggcggtgatggcgggctcaccaccgctgaacgtcagcgtctgaaagagctggaacgtgaaaatcgtgaactgcgccgcagtaacgatatccttcgccaggcttccgcttattttgcgaaggcggagttcgaccgcctctggaaaaaatgatgccactgctggataagctgcgtgagcagtacggggtcggaccgctatgcagcgaactgcatattgccccgtcaacgtattaccactgtcagcaacagcgacatcatccggataaacgcagtgcccgtgcgcagcgcgatgactggctgaagagagagatacagcgcgtatacgatgtacggtgtgcgtaaagtctggcgtcagttgttacgggaaggaatcagggtggccagatgtacagtggcgcgcctcatggcggttatgggactttccggtgttctccggggtaaaaaggtccgcactaccgtcagccggaaagccgttgtcgcaggcgaccgcgtaaaccgtcagttcgtggcagaacgtcctgaccagttgtgggtggctgattctacttacgtcagcacatggcagggggtcgtctatgtggcgttcatcattgatgtgtttgccggatacatcgtggggtggcgggtctcatcgtctatggaaacgacatttgtgctggatgctctggagcaggcgttatgggcccgtcgaccgtccggcacagtccatcacagtgataaaggttctcagtatgtatcgctggcctacacacagcggcttaaggaagccggattactggcatcaacaggaagtacaggcgactcgtatgacaacgcgatggcggagagcatcaatggcctttacaaagcggaggtaatacaccgtaagagctggaaaaaccgtgcagaagtggaactggccacactcacgtgggtggactggtataacaatcgacgattgctggaaaggctgggccatactcctccggcagaagcagaaaaagcttattatgcttccatcggaaacgatgatctggcagcctgagttcacagataaaacactctccaggaaacccggggcggttcacctacagagtaccgctaccacttgtgaagctggcagattcagattgttttcaatcgacaacgcctcatagttaaagtcatcaacgacattgaacgtgtgaagtggtcaacaaaaactggccaccgagttagagtttttccagtatcgattttccgattcgtttggggataacccaccgttatattcgtgcggtcttagtgcgctgtaatatccaacgatatagaccgttatggcgtgagcggcctcgcagaagcttcgtaacccaccaccggcatccattcgttcttcagactcctgaagaagcgttccattgggctgttatcccagcagtttccgcgccggctcatactctgcctgatctggtatcgccacaataactgccggaactgcctgctcgtatcgctgtggaacatcaccccgccgggcttacccgtccagatataggtcacatcaccgcaccacacctgtatagcggcgtaatccccaaaaacgatgctggttgtcagtaaacaagccatgaatgacagtcgttcctgaaatcataagacctggaggagtgtatgctccgtcgcagtaattaccacgatacgcttgcagaattttttccagttactgaagcgtgaacggataaagaaaaagctcttcggaacgcgggaagacgcctccaacgatatttttgaacatcgaaatgttttataacaataagcgtcggcatggttcctgcgaacagatgtcaacaatagaatataaaaacaattattatcaacaacttggaaggagtaagactactcgtggcgattcaatattagatcattgatggagatataaatggctcactttttatcggttgttgacgatacgtttatcaagtatacatgaaaatttcttcataccagctatatgagacgttaagactaataatcttactcccaaaatgactatgttgttgcagtttggtttattgactcttgtctatagttcaccgaatgccaagatgagggagcgaggaactttactgtctacgcctctcttcctggttacaaagacaagcgtaaaactcaccatccattagaactaaaggcactttcctgagcagagttttcatctgcaagataaaagctattttacagccgtaaagtccagtgaaacgaaagagaggttagtgaattaattacttgatgtattaaagtataatgggcgtagataacaccttaatacgaaatcttgtcaggctttcccggtaattagtaatactgttgaaacaaagcgagagggggttagcccaatttacgcaaataaataataatcttgattatgattaattccaaataagtatatttttccagtagatgctgggcattaactaagaaaacccagaataccactttcattgttatggaaaacatggacaatgttaatgaaatatatttctcaccgcgccatcatttgtaccttgacactgttgatcattattatcaccgttttgttcacctttctgagaagctctgatgtgccagaatatattactgccccggtgcgcaaaggcgatatagaaaattctgttctggcgacaggccgtattgatgcaattgaacgggtaaacgtaggggcacaggtatcagggcaattaaaatcactgaaagtgaaacagggcgaccatgtgactaaagggcagttgattgccgaaattgacgacctacctcagtgtaacgatttgcgtaatgccgaagctgcactcaacgaagtgaaagcagaacttcaatccaagcaggcgctcctgaaacaggctgaactgcgttttaaacgccagctccggatgctaagagaaaatgccagctcacacgaagattttgagtctgcggaggcaatgcttgcgaccactcgcgctgaattacattctctgaatgccaaactggtccaggcacagatagaagtagataagaaaaagcttgcccttgaatatacgcgagtcgtagcgccaatggatggaattgttattgccattgtcactcagcaagggcaaaccgttaactcaaaccagagtgccccaacaattattaaactcgctcgtctggatgtcatgaccattaaagcacagatctccgaggctgatataacccgtatttctgtggggcaaaaagcccgcttctctatcttctccgaaccggacaagcactacagcgcaacactgcgtgccgttgaactggcaccagaatcggtaatgaaagatgactctctcgccagcaacacttcagcatcaggctccggaacttcaaatgcgtccgtttattacaatgctctattcgatgtgcctaacccagaaaatcgacttcgtattgccatgactgctcaggttaccctgattactgatgaagcccaaaatacactgctggtacccattcaggcagtgcacagaaatgaggggaagaagcaacaggtcctggtgctggcagcagatggcaggctggaaccccggaatgtgaaaactggtatcacgaacagcgtggatattcagatcctcgaaggtttaaatgtcggagaaaatgtcgtgctgtcgctgccggataagaaagagcccgaagaaaggattatgctgtgaaaaaacttattgagttaaaaggggttagccgaacttatggcaacggagatcaaacgcggaccgtcctgaaaaatgtcgatttgacaatcgttgctggagaaatggtggcaatcattggagcctctggctcaggcaaatcaaccttaatgaacataatgggttgccttgacgtacccaatagaggtgattactacatcgacggacaaaatgccgcctgtctttcacccgatgagctggctagagtgcgccgggaacacattggctttatattccagcgctaccatttgatacctgatcttagcgcacttggtaacgtggagatcccggccatttacgccaacagcgaacgcgacagccgtcggcaacgtgcaacggcgttgctgggcagactgggactggaaggacgtgagcatcataaaccttgcgaactttcaggcggtcagcagcaacgcgtcagtattgcccgagcactgatcaacggtggaaagataattttggcggatgagccaactggtgctctggattcgcaatccgggcaagaagtgttagccattctgaatgaacttaatcgacgtggtcatactgtagtaatggtgacccacgatatgaaggtcgcccggcacgcaaagcgaatcattgagctgtgtgatggcgaaatcatcgctgatagtggaggttgcgtatctgcgacggaaacactaccaaaaaccaacagaatccggcaaagctattggaaaacactgctcgatcgaacgcgtgaatcgatgcaaatggcgctaaaagcgatgaagacgcatcgcttacgcaccacgcttaccatgatcgggattgtttttggtatcgcctccgttgtcactgttgtggcattgggtgaaggagccaggcaagaaacactggaagagattaaaagtctggggacaaacgtggtcagcatttatcccgggcaggatttgttcgatgacagcattgagagtattagaacattggtacctgctgacgcaaatgcactggcgaagcaagggtttatcgatagcgtcagcccggaagttagcgcttcagacaacattcgttttctgggtaaatctgcgatagcgtccattaacggcgttgggcgggaacatttccgggtaaaaggtattgagttgttgcaagggactacttttagagacgatcgcaacgctctacaggaagttattatcgatgagaatactcgtaaggccattttcgacaacacagggttacaggctctggggcaaatagtctttcttggttctgtaccagcacgagtggttggcatcgctaaaagtaacaaccgtagtgatgcatcaaatcgcattactgtgtggatgccttacagcacggtgatgtaccgtattgtgggtaaaccagttctgaccggtattagcgtcaggctaaaagacaatgtggataacgaggctgcaatcagcgctatctcccaactattaacccggcgtcacggcataaaagacttccagctttataactttgaacaaataagaaagtctatcgaacacacctcaatgaccttcagtattttaattcttatggtcgcatgtatctcactaatgatcggcagtatcggggtgatgaacatcatgttgatttctgtaacggaacgaactcatgaaattggcgtacgaatggcggttggcgcgagacgcagcgacattatgcaacagtttattattgaggccgtattggtttgcctgattggtggcgcactcggtattgcactgtcgtatatcacaggtgcgctctttaacgctctggcggacgggatattcgcagctatatattcgtggcaggctgctgttgctgcatttttttgctcaaccttaattggcataatcttcgggtatctcccagcccgcaaggcagcaaggatggacccggttatttcactggccagcgaataatatatgaaaattttatcatcgttgactttatgccttatcaccgcactctgttcaggttgcacaaacgtgctgaaaagtgactatcgcgctcccgaagtgaactatcccatcaactggaccaagggcgatgtggacggaaatacatccccgtttgactgggaagaatttaacgatcctaatctcgataactggcttcacctggtgatgacaagtaataacgacatagctatagctgcactgcggatacatcgggcgcagctggatgcggaaagaacaggtatcaccaatactcctgcactaaaggcggcactgagtatggacggaaaaaaacagctgaataactcgtccggctgggcaaaaagcggttctgccagccttggcaccagctatgagctggatctctggggaaaaattgcccgtcagcgtgatgttgctgagtgggcagtccatgccagtgaagaagattttcgctccgcgcgcctgatgctgctttcagaggccagcaataattactggcgtatcggttttgtaaatcaacaaattaccaccctccagcagagtattgactatgcaaaagagacgctgcgtctggccgaggttcgctatcgtgcaggaaatatctcgtcgctggatgtgattgacgcgcagcaaaacctgctcacgcaagagaaccagcttacagggctacaacgagaacattcacagctactcaatcagcaggccgttttgctcggcaccgtgcccggttgccagatagtcgagcccacaacgctgccaaaaggaagcctgcctaaggtcaatgcgaatattcctgccagcatactgatgcgccggcctgacataagtgctaaagagtggcagttgcgtgaggcgctagctacggttgatatcaagcgtagcgaatattaccccaccttcaacctgacaggcgctttgggtaccagcagcgcttcgctactggcattactgcataatccggtcggttccgtaggcgctaacctgacgctaccgttcctggagtggcgacagcgggacatagaggtaaaaattgcccgtaacgattacgaacagcgagtgctggagtttaaacagctactttataaggcaatgagcagtatagaggacgcgctttcatttcgtaaccagttgctgcttcaggagacaagactcagggaagaactggaacttgcacgtaagtcagagtggttaaacgaagttcgttaccggcatggtgcagtacgtatttcattctggcttgatgctcaggaaaaacgtcgtcaggcggagctgcgactggatgaaaaccgcttcaatcagttgcagaatctagcaaaaatttatcttgagtttggcggggcatcaacctttccttaagtctaattggggacaaccaggagcaataaggagtgaaaaatagccacagctgaaaacaccatagccattgacatttgagctttacctgctcttgaaaacaaaaaatcactgttaagcgcaatgctattcacttcagcagagcagtattgcgtgtcacgagcatgtttaccattctgtgtaaatatcttttcttaaaagtgatcatcaaagcagtcatcaaactcgataaatatccaccggcatagcgtaatgccgagcaattaaggatcaattgaccgctccttaaactgcggcactataacggcttctacaacagggagccgttttcttgtgcatagaaaacccccagctaggctgggggttccggaaagctttcagctttgagccagttattaaaaccccttttgatttattaaaacaccttgcggtctggcaactgcaagtgtcaaacaagaaatcaaaagggggtcccaatggggaacgaaaagagcttagcgcacacccgatggaactgtaaatatcacatagtatttgcgccaaaataccgaagacaggtgttctacagagagaagcgtagagcaataggctgtattttgagaaagctgtgtgagtggaaaagtgtacggattctggaagctgaatgctgtgcagatcatatccatatgctacaatgtttgtcaaagctcagtgaaaatgtctaatcactggctcattataaatgtccatatattgaaaaggccgctgcgaaaatccctcccgcagcggcctctttagcataacgtcattatgcgaaccatcacttaatgttcttctcgcattttaagagaaatagtaaatttgtagcagtaagatgaagcatatagcttaccaattcttccggaacctctttaatatcagctccttgcccatgacctgcatttttgtttctgatggttggaattcctgtttccagcattgttttcagacttgtgaactggctttgtaaatatgccggaattaaattttgacttaaacagctatttattagttttgatgctgtatcattaggactatatttccagttattcttatcatgtattgctttcattatactttcaaatgatttcagacaatcatttagacattctttataacgcttatgcctgtaatgctcatgcgcagcaaggaattcatcattggcgccttcaaacaagggttctccacttaaaagaatcagggttggctttacgacatcagcatgaatcaattgagagtctatccttattatctcttcagattcaaactgataaccaaccccatgttctctaaacctttcatttagttcaataactgcatctccaggactttgacttgttatgtctttgaattcataatggtttttcgcaacgtggctcactagtatctggaagcatatttctatgaaatctaggcatttctctgtatttctctctttaaggaaaaagtcgacaagagcgtcaaagtcattctttgcaaattccttcaaagaaaaaacaccatattccttagaaagtatctcatggatatatgcatatactttgtcagcttcatttctatacgatgtgtagcgttcattagaagaggggaagccgatagaatcagttattattttaataatttgcaccctgagtgcatgtggaatattgttatattgatatacgtcatttacttcgccacgaatttttctttgtctcttagaataaatattaataattgccatgaaatagcctcaagatttttttacatgctgttctgtgatacttgctgtttggctctttgcaccgcgaagcggttcggggttctgtgacacccgaattttacctcggtttcctttgtatataatatcattgaagttatttgcatttctcagaaatcttctctggctttgattgcaatagccgaaagatagcgcacacatcgccgttggtcagttagtacgaggtttccgccaagttcaccttttaccaaagatggcctcagagcttccctgaagctttatcatggcttcaagtaggtcatcttcgttcagtgagcgctgagatttgttgatgctaggggcgtgccgtctgcgggaaggctcatcaccagaacaaggtaacgattgtgagcgattattgtcccgttttctctggacctgacgggcaacatccaacacatggccaagtctcttgttatctacaatggcgacgggatcaacctcagaaagccggtcataggtggagtagggcagcagcacgccatttagtcgcagctccttatgtccgtcaggataatgccagacatcaatatacttgccgatggctcttcgactgtactcgttgtcctcaatcagatagagcattttatcgtattgtacagtcagattttttgatacccggcgaggctctcgctaggtaaattctgcatcaaggtcatcgtctaatgccagcggccggtgcgcgttaaaatcatgcaagggttctttggcaaagcgccggttataatccttctgcaaatgcattggccgcttctattgtacagatccccttaagcctgagctccttaacgagtcggtcctgaagtgtcaaatgagcacgttcaacgcggccttttgcctggctggtttcagcacacaaaggagtaatattaaggcagcgcatggccctggcaaactgggtttcgccggggccggtggtggcgtgcttattattgactctgaatacgctggctttatcgctgtagaggatcatcggtttaccgtatttctcgatatagccacgtgtagcttcaaaataggtaaatgtggactcagatttcacaaacagcaggtgcatcaggcgactggtggcatcgtcaacatagaccagggcagaacagggtcgaccccggttttcaaaccagtggtggtcgcagccatcaatttgtatcagctcgccagtacaaggacgccggtaacgaggctgatgaattttcggtgcgcgttgtctgcggggaatccagagtccggcctttatcatgagacgtcggatggtttcctttccaaggaccagaccgtgaacttcctccagcttttcccgcgctagcgtggggccaaaatcacggtagtgctcccggattatactgagtgcctgatctgtcagtgacgctggcaacagacggttaccagtgcggccacgactctggttgttcatgccaagcggcccatactggcgataacgtttgagcagacggctgcaatgacgtggcgtgatgcccagcatttcggcagcctgtccaggacggatattgcgatcgatgacatcctgaaggattttgaggcgattgatttctttcacggtgaacattccggcgtcatttatagccatagaaaactcctcatcaggaaaaagtgagtcgttgtctagacatagggatctgagaaactatagcctggacattcctaatgagccacaaatagacattagcattgagcctctacaatgtttgtgcgcacaatctatcttatgttaaataacattgaatattaacttacttcatgaagcttactgcttctatttccacttctttttcttattaatcgctcccctaatccacgcattcatctgctctacaggctctaaaatattttttctgagtactgagcacaaactgctccataaaaaacagaccacaagtttgggcatgatactgatgtagtcacgttttattgttttcatgaagctctctgctattccattactcaccggactccgcaccgccgtgttcttcgatttaagtcccaacatccgagcgaactggcgtgtttcattaacccggtagtcagagtcagcggtcgaaagacctcccaggtagctactctgcatacccactataatgcaactattctcgtgacgtgtgtttcagctctcccccggctatgatacgagatggtatggagagcccttctggggccactgcctgcaccagatgcaacaggaagcctgatatgaatgcaatttctgtcaggacaggcgggtaactttctgtggccatggatcagggaatactccacccgacatccaccactgaatttcttcttcagtcaacaagcaaagggataaggcatgtgtaaaatcattaacttcagtggcattgccaatgatagtcaactgacagcagcgatcgccaaatcgcccagaggctgtgtcgatgtgtcgctgtagcgctgaacgttcttcacgagttaaattattgtcagtatgttccagtactccagctttccagtagccaatgagcgccagactgatgcttcctgctgactgattccagagcagggcaagatcatctctcccggggagccaaaaaaaacctttactgcgataaacgcccatgcccataaagcggtgacaggtatcccataatctttgcggatggaatggcctgtcgtccctgatgacgcgccatataagttcctcattgttgcacggagtatccatgtttcctacaagtgcttccagttcactgatgagtaactcaacacggtcaaaattgtagtccgggatcgccagtaatgtgctcagttcaatatttccccatgataccgcgagcacatccacggaggggttaagtgggtgtatggcttttgccacagcggagataatgtcaaagggcaggcggtcatttctggtcagcagcagcctgttgcagaacatgatttgctcggccagcaaattctccaggccgcgtttgttatgccggagattttcctgaagagtggggatcagttttttcccatcattgtaatcttcatggagcatgacagtatcaacgagtgttaaaaacgccttcaggcgtacacgtgaatggcgacgcaggtatttgaccagaggaagcggatgactgcttccggaggtttcaatcagcatcacatccggcttgttgtgatcaagcaggtggttaattgcgagatccatctttttgaggcctgcggggctgctgatgctgtctgcggtgatacttacgaaattattgtcttttgcatctacaatatctgtgttggcgatcagcacgccgtccacatccagttcgctcatgtcattgactatgacagagactgaaagctgtttttcgtgtgcctgtatgagcaggtttttcagcagagtggttttccctgcgccgaggaagccattgagaatgatgacagatgttgtttccatatcagcagtacaccagttagttagcgaggaaggcgcttggtggtacaaagagcatccggattgaatcgcataacgtcgacgggagttgttgtcgtttttcctgtgaggtctcaacctgccacagaacgcaccggatttattggtatttagtctccatcagcataattatcaccatttttatctccgggggaagatttgttctgaacaggagtgggaggtgtgacctcctgaaagtaattacagattgctagttgttgtatttttattcgaatatgtctcttcgtatcagtgatagcacctagataaagtatattccgggagcgttatctgttctagatatcagagagtcagtcatttgtaaaaaagagagctgcatcattctatttttcctgtctattcaccctgttgtcgtatctaaaagacatccgttccgaggatgtcgggcacccgttctgtactaagatacatagcttttgggtctgcactatttggaaccgacagaagtctgtctgtgatatccgcatttttctgtgtgcgatatattccgaaaaacttcaaaccacttaaaagaggtgtttccggaggataagttcgaccagggaatagtgtttcctggtaagtgtggcggagccagttcaccaggagtatctttcttatccagttatactggattttattagatgatcaccctaccctgcagcagtacgctgacggtgtttttttgggagaaccattatgaataacgtcatctttgattggtatttatctaggcaaaaattcattcaacgtccactgccaagatgcaacaggcagagtccttaactgtcagaatccaagtgctcgaaattattcgaattccttactaattcctcctacacaactattgttatggaagcttgcgaagatgctcactatatggcacggaaattttcggaaatgcgtcataaaacaaagctaatacctcctcattttgtccaacccttcgttaaaattaataaaaatgatttctttgaggctgaaaccatatgtgaagttacatcaagccatcaatgagatttgtagcattaggagctgaatcccagcagaggattccacttttcatccgtcgttgacaatggccttgaaaaacgccgttactcaccgttctccttcacggtaatacgcagttatagttgcagagcaatgtaatggatcttttatattaagaaattgtgtgagtggagaggtgcatagatttgcccctatatttccagacacctgttatcacttaacccattacaagcccgctgccgtagatattcccgtggcgagcgataacccagtgcactatgcggatgccattcgttataatgctcgaacgcctctgcaaggttctttgctgccgttaacccgtctggtttgggcatgacactgatgtagtcacgctttatcgttttcacgaagctctctgctatgccgttactctccggactccgcaccgccgtgctcttcggttcaagccccaacatccgggcaaactgccgtgtttcattagcccggtagcatgaaccattatccgtcagccactccactggagacgccggaagctcgttgccgaagcggcgttccaccgctcccagcatgacgtcctgtactgtttcactgttgaagccgccggtagtgaccgcccagtgcagtgcctcacgatcacagcagtccagcgcgaacgtgacacgcagtctctctccgttatcacagcagaactcgaacccgtcagagcaccatcgctgattgctttctttcacggccactctgcctgtatgtgcccgtttcgatggcggtacagcaggttttcgctcaagcaacagcgcattctggcgcatgatccggtaaacacgtttggcattgatcgcaggcataccatcaagttttgcctgtctgcgaagcagcgcccatacccgacgataaccatacgtgggcagctctctgagcttacccagcaatagtggacacgcggctaagtgagtaaactctcagtcagaggtgactcacatgacaaaaacagtatcaaccagtaaaaaaccccgtaaacagcattcgcctgaatttcgcagtgaagccctgaagcttgctgaacgcatcggtgttactgccgcagcccgtgaactcagcctgtatgaatcacagctctacaactggcgcagtaaacagcaaaatcagcagacgtcttctgaacgtgaactggagatgtctaccgagattgcacgtctcaaacgccagctggcagaacgggatgaagagctggctatcctccaaaaggccgcgacatacttcgcgaagcgcctgaaatgaagtatgtctttattgaaaaacatcaggctgagttcagcatcaaagcaatgtgccgcgtgctccgggtggcccgcagcggctggtatacgtggtgtcagcggcggacaaggataagcacgcgtcagcagttccgccaacactgcgacagcgttgtcctcgcggcttttacccggtcaaaacagcgttacggtgccccacgcctgacggatgaactgcgtgctcagggttacccctttaacgtaaaaaccgtggcggcaagcctgcgccgtcagggactgatggcaaaggcctcccggaagttcagcccggtcagctaccgcgcacacggcctgcctgtgtcagaaaatctgttggagcaggatttttacgccagtggcccgaaccagaagtgggcaggagacatcacgtacttacgtacagatgaaggctggctgtatctggcagtggtcattgacctgtggtcacgtgccgttattggctggtcaatgtcgccacgcatgacggcgcaactggcctgcgatgccctgcagatggcgctgtggcggcgtaagaggccccggaacgttatcgttcacacggaccgtggaggccagtactgttcagcagattatcaggcgcaactgaagcggcataatctgcgtggaagtatgagcgcaaaaggttgctgctacgataatgcctgcgtggaaagcttctttcattcgctgaaagtggaatgtatccatggagaacactttatcagccgggaaataatgcgggcaacggtgtttaattatatcgaatgtgattacaatcggtggcggcggcacagttggtgtggcggcctcagtccggaacaatttgaaaaccagaacctcgcttaggcctgtgtccatattacgtgggtaggatcactccgataacatggtgtatacggagaagcacatccgtatcatcagtgtgacgactgcggcggccatccatccagtcatcggttcgtctgagaatgacgtgcaactgcgcacgcgacacccggagacaacggctgactaagcttactccccatccccgggcaataagggcgcgtgcgctatccacttttttgcccgtccatattcaacggcttctttgaggagttcattttccatcgttttcttgccgagcaggcgctggagttctttaatctgcttcatggcggcagcaagttcagaggcaggaacaacctgttctccggcggcgacagcagtaagacttccttcctggtattgcttacgccagagaaataactggctggctgctacaccatgttgccgggcaacgagggagaccgtcatccccggttcaaagctctgctgaacaattgcgatcttttcctgtgtggtacgccgtctgcgtttctccggccctaagacatcaatcatctgttctccaatgactagtctaaaaactagtattaagactatcacttatttaagtgatactggttgtctggagattcagggggccagtctagtgcacgaatcctagaggcagaatgtttctccacatgcttgtggagaaaccgccgcattttgcccgttctgtacgatactccgtagccagagataagatattctgttgctgtatgctacatacattacactatacgaaatcaggtctgttcaatcagtaaggaaataaacggacaaacgttatgtaacccagacagagatatattgtctgggaaaatagtggcttttgtggcgaaaactagtaaaacagcaacccgaaccacttgatgtgcatcgtttttgattattcccgtatactcttgcagaaggagttctccgtcgggctactgtcatggttaatgcggggaatatggcgacaatacaacacacctaaaagagtaatggacagatgaagcggtttattcatttcccatgattctgagtacctaccaagtctgagtaaccacttttatacttttaattttcgttcatttagctatcgtttaattattatcacataggattctgccgtttttaacaatgcaggataataagatgaaaaaaatgttattttctgccgctctggcaatgcttattacaggatgtgctcaacaaacgtttactgttggaaacaaaccgacagcagtaacaccaaaggaaaccatcactcatcatttcttcgtttcgggaattggacaagagaaaactgttgatgcagccaaaatttgtggcggtgcagaaaatgttgttaaaacagaaactcagcaaacattcgtaaatggattgctcggttttatcacttttggcatctatactccgctggaagcccgggtatattgctcacaatagttgcccatcgatatggggagctcatctgcactgttcattaatatacttctgggctccctacagttgtttttgcatagtgataagcctctctctgagggaggaaataatcctgttcagcgatgtctgccagtcgggggggctgcattatccacgcccgaggcggtggtggcttcacgcggggatgggcagattgatctgatatgcaaccgacgacgaccagcggcaacatcatcacgcagagcttcattttcagatttgggccaccttttgatttctcgtttaacttttgcagtcaccagactgtgatttattttaacaaatcaatagctttcataaaattctcgaagctgaaagcttaactgaaccaaccagcctggctggtgttttctaaagacaaaaaggccatcagcaaaacactgatagtctgaatcacctttatcaatcatgtatgaagaaaatatgcgcaattagttgacagtgattatcattttcattaaaaagagcgcgtagcattcttatttcatgaggaaatttacccgccaacaacctgagtagcgaaagctgttcatccccaatatttttgctggcgggttcttttttctactgtcctcctccccagaggaagcattcccattaatcattactggcgaggaggcaagagaaaaagtcggttctttgtccctaaataacagcatcatttcagtccatgcaaaatctgatttaccagctcacataataacctgatgatacacagtgtaagttcacagagatattgcaattgcctccggataagtaaggggagattgcactatgcaaatgcagcacctgaggttggctatcctaagcagccgattatgcgttgaggctttcagtgatgggatatagcaacaatgagaatgaaggctctgcacttctgggataaacacggtatttctgcagcttctgaagcgtttggcgtatcctgccgcacgctttactggtggcgtcagttactgaacaagggaggacctgaggggctaatcccgcacagtaaggcacctctggtgcgacgaaaaaagcactggcatcccgatgtgctgaaagagattcgacgcctcaggacagagctgccgaacctcggtaaagagcagatttttgttcgcctgaagccctggtgcgcataacgccatcagacctgcccgagtgtttccaccatcggcagaatgatcgccaccgcacacgataaaatgcggatgataccagtacgtctgagctcgcggggtaaggctctgcttgtcaaaaagcgaaccaccaaacccccgcaggccaaagcactaccgtccggtaaagacaggagagctcattgggatggacgccattgagctcagaatgggtgaaatgcgtcgctatatcattaccatgatcgacgaacacagcgattacgtactggcgctggctgtgccgtcgctcaacagtgatatcgtcaatcactttttcagccgtgcagcccggctgttccctgtcggtatcagccagatcatcacagataacgggaaagagttcctgggaagctttgacaaaacgctgcaggaagccgccatcaaacacctctggacctatccctacacgccaaaaatgaacgctatctgtgaacgttttaaccgggcattaagagagcaattcattgaatttaatgaaattttactctttgaggatcgcacctgagg

SEQ ID NO: 2 - DNA, 71719 bp, synthetic construct
  source 1..71719: mol_type = other DNA
gtgcgaatgcctccggcgcgggccggattattctgaggagatcactttcagggagaagctgtggccagccggctgtaattacggttacgtgacagaatcatgcgctccttcacacgacgctccacttcgcgttttaccgcctcaccattagcagtgaagcgtccttccgagatttcacgcgtcagctgccgtttcaccagggtgacgatatcctgacgttccctgttcgcatcacgatgcgcacgggcacgttttattccacgggacttaagctctgtctggtaactgcggaaacgctcacgcacaaaacgccaggctttcgctatcagctcatccatacccagggtatccagcccctgctttttgcgctgtttgttttcccattcaacacgactgcggcgcgcagctgccactgcatcctcagacacatcaagggcagcaaacagagccagtgtgaacgtgatgtcggtcggaatgtagcacccgataagcgggtcatattccgtctggtaggtaatcaaccccagctctgacaggaacgtcagggcacgggtggcacgggtgatggagagttttccggcagcagactccgtcgccagtccgcactcaatggccagcgtggtgatggagcactggacgcggttggccagcgggtcatagtggaaacacagcccctgcaggagcgcatcaatagcccgtcgacgcagcaccggtggcatgcgtcgacgcagaccacgcgaacgggcatgcgccacatgaatggcgaaatcaaaacgggaagtgaagcccaccgccttttccatcagtttttcgcagaacttcagcgttccggcacctttacggggtgtaaacaccggattcgggttctttacctggcggtaatacgtttgttgaagatcagtcacaccatcctgcacttacaatgcgcagaaggagtgagcacagaaagaagtcttgaacttttccgggcatataactatactccccgcatagctgaattgttggctatacggtttttagatggccccggtaatctttcatgtcgccaaacttgaagaagattatcggggttttcgcttttctggctcctgtaaatccacatcagaaccagttccctgccaccttacggcgtggccagccacaaaattccttaaacgatcagcaatctatcacgcacaccagaaacaggcaagaatgtgatgtcgctacattttagttagctaaaagcactcttctgctttatcagttcggcaatcatctcagcctgggtaacgccctcagcttcacacatcacctgcaattgcgcttttaacgcagcaggaatgaacactctgagttctttatgtgtagctcgttttcttgccagggatgcctgctgacgttctgcaagcgtcagaggattaccttttctgtatgctcgctttgtacctgatgaggaagtcactgcattttctgtctgcgacatctcgcctcctcaatactcaaacagggatcgtttcgcagaggatactacagttttttgaaatcagcgacttgagaattgtgacgaagatctgtgttggcgcacaaatcaacggggattactgtcgtttaatgtgatttaaactgtgaaatagtatggttttcagttatggaaacgccgggagcggggaaaacttgctttttcccgtttccggggttggacaactgagcaacgcgaaggcgtcagctacgatgttccggggactgctgatcagcccctgttgtattcagccattctccggtcatctgcagcagttttctggcatcgtggcttctgagcaacggaataccagcctgcctcagaacctcttccagaagaatatcccggcgtctgcgttccggtcgtaaatggctggcatcatccagctctactgctgcaacaatactgaacgaacgccgctcaacgataaccacatcaacatgccactgagacaccatcctgaataactgccaccactggcgtgatcgtggccggatattcccgttcagctggacgatatccgcgacccgtacctgcggacacagataccaccgcttcatatccacaattctgaacagtcctttcataaaactgacttccctttctgtcagcagcgtcttctgacgttgccagtattcaccgccggacagtattcgctctgcatcaccggtccgtatacctgccgcatgcaactgcatcagcaaaggagaatgctgatttacacttctgaggataatccggataagtaacgctgcaataatcagaacaatgagaataaaaataatcagcttcattgtttatctgctcctggcaattaattattgctgaattattaccttacatatttccatatctcttttggtatcctgtcatataactttccggtgattaattccgctaaccggtggtctgctgctgaattaaatgattcacgttcactgtttaataaactgtctctgtttttatgaataattttttccagcgtattgactgatgaacatcgccgtaactgatacagccattcctgttttgtttttgccatttttattcccgctgagaacagatgaatgataagattgccagcgctttatttacggtgtctccatatccaggggggaaccggatcagcatctgaccagagcccacgtttttgttgtctggcttcgttctgaaagacaggtagcacgggatctgtgttgtactgctcatatacccaggcagccccggcccggaccataaagtgattaacattcattccatctggtgcataaacctgcccgagaatacgaccataacgatctcgctgaaaatatgtgacggtaaccgtttttcctgcaaccagtgatttcatcatgtccgttgaccaccggccataatcctgtttcttttccggtgcatcaatatttaccagcctgatccgcaccgccttccgtgagtccatgacctcgatggtatcaccgtccagtacacgaacaacccgtccatgaatatcttcagacaataccggccatgaaaagataaataataccagtggaatatatttcatcatgttattctccatatcagataaatggcacctgtatttcaggcatcctgaacattgcagcacacaatgaataacaccttattttgtgaaataaatcacaaaatataaaatcacgacttaatacggaaaaaactctgtgctactcagaactgtccggtcatactgcccgaccggatattaaaaaaagcaaatctcatgcattattcagccttctgaatccctggagtaatgaaggctgctttttcgttacacccttccccggcagcgccggtggcattgttcagccctttttcgggcaaactgctgccagtctgtccggggtgtaacaccggcagcccgtcgccatgcttctgccgcttccagaataacggcccggcttccggcactctggtaacgccagacctgaccgttccgagaactgtttctgaatccattcattttcctgtcccccctttagttgtcagtattattgtgataataaaaataatcagtttcaccatactctttcccttttttatctgataaacggatactgaattaataaattcattttccttgccaccgacttattctgtttcttatcaatgcatttcctcacagaagaattaatgctgccgggatattcatacacaccacatatactgctgatgtttcagtgattattcccgatgctgaaacggaaaggcgcttttctttcccgtcttcttaagcgctgagcacccgaatctgtgattcaggcgatgtttttctcctccatatattcacttttttcaccatgtctggcaaagtgaataatgctcatatcagatcctgctcagggaactggctaaatggaccggaccggggaagcaattccccggtttttctttgccgcgcacattttttgaattaaatcacacattttattgttcctgcttgtcctgccaggaaaatatgttacaggtattgttacaggtattatgtcgtttagtccgattcagcttacagacagggataaccttctggttcaccataataacaatcatccttaattccccttttcagccccttcgctccgcaaccgcaacgggggctgtttttatctgagtacagctgcgatacctcatgctttattctcctttttcgcctttttccagattatctttttctccctgacttctgtgactccccgacgaaccagttcattccttatcgccactgcccgggttttgttccattcgtcacgcatgtgtgactttaaataattattaaatgtttcgacggacacaggcatattaccggctccgctgccggctatctgctgcagctttcctgcccaacggctgacgatatcactttcgggaacagaaggttgtttcaggcagttacttttgtctgcaccagactcaccggcagcagcaatactttccggtacatcacgacggatgcaggtaataccggcttctttcagcgtcctgataacaccgtcatatcccttatccgatgaaatgatgtggtatgttttatctttccctgctgcacaacagcttatccgccccagctccagaacaagatgaaaatcgacattattcctggacacaccactcacctgcctgatagtgattctcgcaccttcaggaaaagagtctgcaggaagaacaacgacgttctgctgtggcccgacaaaaataatcagttccccatactgctcaagggaaatatttttcagactgccacagttttcataatcaataagggctgtcagggtgtttttatctgtactcgtattattttcttccatgtttttaccttttattcataaccgggtgagcaataacacaaaatattttatttccgggaacaccatataaaaagaaagacttccctcgtgaatattcaggcgcatattaaacgacgtcttgttttatgaaatcaatcacaaaacagcacgttattacttaatacagcaataatcctgtgataatgaatgctgactggtcatactgccaggccagatataaaaaagaaaaatctcatgcatacttcagccttctgaatccctggagtaatgaaggctgcttttttcgttacacccttccccggctgcgccggtggcagtgttcagccctttttcgggcaaactgctgccagtctgtccggggcgcaacaccggcagcccgtcgccatgcttctgccgcttccatccagcgtccactgacctccagaataacggcccggcttccggcacgctgccagcgccagacctgaccgttccgtgaactgtttctgaatccattcatctttatcactcctctttaacccgtcagtattatggtgttaaataatctactgaattggcttcctttcgggtacggaagttatttccttttaagccttatttccctgcctcctctctgatggaggctttttttatttctcatcaagcacggcctgaagttctgcctttatccggttctggcggcggattttatccagacgcgctgccgcatacgcttcctcctcctgagaaatatgctccgtcacatacccttccgtgtcataccggcaggcaccggctttcatggcacacagatagctttctgaacgggtgatggccttcagcgccctgcgcagttttttatgcgagagcgggatattccgctgcgccacgtcttccagtaacacgtcccggataccgcaggccagaagccggggcgtgtcaccgtcaaacaatcccggccaccagggtttcagggtattcacggcctcatccagcgtgggcaggttcagataaatggacagtgcctgtctggcctgcgctttttttgccgccagctctgcttcccgggctgctttctcggccagcttctgctttttcaccttccactttggtggcgtggtgacattgatgatggttttccggctgcggacaggcgtctccccttccgttttccgcttcagtgtcagtaccggtcgcttctgctctgtcatgctgtgttctgcctctgacgaatcatcctgttacggtcaaagataccacacccgtcacagcgccagaacgcccagcacagccagatgacaggcgtagaataccgggaaaaaatccccgggccagaaacggggtaatgattttcctgcacaggacaccagaccaacggtcagcaccgtcatcacaagaccagccaccgccgctgcatcactgctggcaaggttaagcgccggaataacagccagcaggcaggcgaccagcgccagacgttccgctctgtcttccgcccggtacagccggtggctgactgccagcatcagcagcccggcaatgccgtaactggtgccggacaagggcccccacagggccatcagaagaatggccgctgctgtacgccacccactgcgcgtttcacaccacgtcagcacctgtgccgccactgcaaaggcaaacaggatattcccctcataccagggaaagccggccagataatatgcgaactgggcaataatcccccatccccacagccggttaatggccggttgccggatattcgcatgacgggacagattcagcccccacaccagggcaaacagcggaaaggctcctcgcccggccagaaacatccattcctgcttcagctggaatatcagattgatgtggtccagtaccatcagcaccagtgccaccgttttgatgatatcccgctgtccgggtgaccagaccagaaatgactgcaaccaggtatcggtccgtgcagccagtgaatcgttacgtgtcgtgttcgtgttatccgttgtcatcagtgtttgtcttcctgtatcagtctccgcccagggttttctctttctgcaggtcacgaaccatatcccgctccatctgctgcagacgctcctgcagcagattttcccgggcaacctccctgaccgcttcctgctcccgttgtgattcacgcagcacactctccggcagcgcggcttcccgttcagttatgtcagcccggtcacgctcctgcccggcaatttccctgacagcctgctctgttttaccatccggcaggtcaggtttgttctctgccatttttctgacaatttcatctgcgcggcgttccgtttcacggcggatggcctcttcagcctgccgctgtgccagcacctctgccgtgaccggttcgccatttccggctcccggctggacgctgctgtccgggatatccccccacacgcgaccaccggtgatggcaccgggattccacggacgaccttcaccggcaattcttaccaccacaccgctgtcaggattatcacgggcaatccggacaccatcctgcatattatcagccagcaggctctctccgttacggctgccctgcagggccacaaactgtgcatcttcactgcccttcacccggccttcaccgctgaatccccgcagcccgtttccgtcatccgtggtcagtgggttcagccagataccggcggacctgccgttacggtcaaacgccggcagtgccacatacggctgcggatatttacgtcccggagcaataaaccgtgcaggactgtctcccccggccagccccgcctgacgaagaacggctcggcctgccgccacgtcccgcagctcccgcgccgtactgaacagccgctgcgcattcatgacctcccggtccggtttcggctcaaacacatcgtgggcagttcctttctgtacggcattgttaatggcatccgtccagccctgacggttatcggtgtacacctgcacatgctgcttcatacgcgacagggccacgtaggctgactcaaagccggccatcagtttccggttaccttccgtgccttcaagcgcgatggcaaaggtttcactcgcaccctgcgcaccgtgggcggtgatggcataggccaggtcaatatgttgctctgcccgctcctgtccggggcgaatcacccgggtctgctgtccgtccgacagcgtgacactgtcaccggaaaccgctgtcaccgtccagacgctgttggccacataaccgcgctcccggtcactcttcgtgaagcgcatccggtcaccggtccccacccggatggtgtccggggtgtacagtgtgacgccttcagccaccgcctcccggggcgaaatcagccgcgtgttaccttcagcatcctgcagggttatcagcctgtcatccttgctgataccggcaatccggtgatacacattatccaccagggcaagggcatccgggttattctcccaggtggagagacgacgcagctccccgtcacgtatattcgctgtgttcaggacaggcaccatgacctgctctttccccagctcaccggccttttcccgtgcatcatgaatcatgctgttcagtacgcgccggtcctcattcaggtgcgtgacaatcagcgtctgctcccgtgcttccggcgtcctgccggtatagtcgcggacaatggcttcatacagtgtcatggggacatccgggaaagcctcgcctttcagcatcgccttctgctgcgcttctgccagtttcgcttcctggctgtgactgaactccgtcacggagtgctccggtgcccatgcgccctccagacgtggcacctgagacggtttcacactctcaagcccggacagtgccctttccacattccggttaatcaggctgtataccgcctcccgcagttccggcgtctgacgcacaatctccttcatgatgaccacatcggcagcactgcgcgtctgctggagacggaaaggctgaccgggcgcgatggcctgcagctggtccgtgtcaccgctggccacagcacgaccgccaccggccgcaatcagggcgtatgcccgtgccatgtcggtattgcccaccattgagctctcatcgagcaggaacagcgtgttgctgaaatccggcgtttctccgctgcgctgctgcagctgcgtgtcatgcagaaaggacgccagtgtctgtgcatccacgccggcgctgcgcatctcaccgaccgcacggtgcgtgggccccagccccacaacccggggacgctcactcgccggcagcatgttcacggctgacatcaccgcccggaactgtgtggtcttacccacaccggcataaccctgtaccaccgtgaaacggtcggacgtttccagtatcatacgggtggcggcacgctgtcccgacgttaacgtctccatgagttcgccgggtactctctccatcagcggcgtgaccgcctccttaccttcgagaatatggcgaagaatacttttttctgcctcatacgacgcacgggaaaccagcaggcctgtgccatagccttttgccacatccacatacagtaagtcaccgcgttttatctgcgcattgatttcccctcccagttcagtaaaaccggttccttctgcagcaaacgacttcgcctctgtcagcaggtccaccatgctgaaggccaggtttttactttccagcacaggaagggccagatgaatggcctgctgtgccggcgtgtgcagcccggctttctgcagactgatagcggtttccagcgatgtctcaccggcccgcgcctttatctgctcagaaaccaccgtaaaggagggatggcgggcaagtttttccgcagtacgggtttcatccagtgaggaatacagccggacatcacgaccgctgcgggccagaccgttcagggtggcgttgtccattgccatctgtgtgacggaggcaaacaccgtcgcactgtcactgacggaatgcccgggcgtttccacccagccgttctccagcttcagtgccgtgaacggtgaatcaaccacaggcagggtggccggttcagcccgccccggcacaacaaccgtcatcgcctcttcactgacggatgccacctgcaggcggtcaccgccggagacgcggagtccgggaattttccctgtcaccctcagtcgctcgccgtctgccaccggcattttttccggccggaacagcgaccagctgctgtccagggaggaaatacgcaccacctgcgtttcgccctgcgcatcccgcagggtcaggctgtgactctgcgctgtcacccggtctatcacatagcggtcatgactgcgtgtctccgggttccactgctccatcaccatccccggtcggtacatatcccgcagataacggctccggctgtccagccagaccggtgacagtgcagtcatggtcacctcagggtgtccgagcacgccctgcgttttcagctcactgcgaatggcctgtgtcagtatggcctgttcccgtaccccgctgacctgtgccacgctctcttctccggctttcacgctggccgcaaaatctccggccagccgggcatagcggacattacggtccggttcactgatgatggtggccggtcgctgttctccgccctgccagcgatatgtgttcaccccggcatccttcatggccatcagcgcactgcctgtaccggttcgctgcccgctgtcggttatcaggacctgtacgttatggcgtgcggcaccatccagcagggttaacgtctctttcagggagagtttttcgccctggtcgacgataacggtactgcccggcgtgaaggccatgccttccagcagctgacgacgtccggttatcagttcaccggacagccgttcatcctgcttcaggttcatctgcgagcgacggtcagcagcgataatctgcacctcccgcccctgttcccgggccatcatgaccagttcagccacccgctcacgctgcccggctgcaccgccctgcccggacacaatggccagcgacgggcgatcctgtgccagaacactgacggcatcgctgtaaccggccgtccgggggacacttttctccggatgtacggtcacccggttctgtttcatgatgtcacgactgagtgcccggactgacagctcatcgagcacatgaatcccggacgtgaacagccccttctcacgatcgagggggataagctgctcacggctgatggcctcatcgataccggcgcgtgcccgttcaatcacaccattttccggcggcagtatgccgaccgtcctggccagcacgtccgtgtacgtgaactgcacttttcgttcacttaatccggcaatcgcctgtgtcaccgcctgctgcacatccggcccgtcctgtgaagctggcccgggcgcctgcgtgcggatctccgtacgctgatccgccgcgtcacgatatgcccggatgtcgaacccggtttccttcagcgtctgcatccattcagccattctgatttccggatcgacgtgctgtttggatttacgcgtatccagtgccgccacatcccgggatttcagcgaggcgtcttccccgacggcctcccggatagcctgtgagcgtccggaaaaggcctccaccggtacgcccggcatctcccacataccgtgcttacccaccacttcggtttcatagcccagcgcctcaacctgctctttcagtttttcccggtagagcctgccaaaggcaatctgattagcgtacacattctcaatgaaccccgttttccccactttgtcactgctcagtgtcttccactcaccgttatgctgcgtgacattagccaccaccgcatgcgtgtgtaactgtggttcctgatcgcgactggtgtcgtggttaaacagtgccatcaccagattaccggtcagcaccgtttctgactgtccgtccgtcatcacccgtgtggaggccagcgcctccacctgacgaacagcaaagtccacggcctggttatgtgcatcaatcaggcgcttatcgccacctaacatggccatcatggagacacttttgggggcggagaaggtcaggtcgtagccgggacgatgcttattactgccatcctgcatgcggcttagatccgctccgtccggcagccttccctccagaagacgggtaaaaacatccttatcgacgctgccctgcagcccaagctgttcagcccccttgccggcccagcgttctcccatgctgcccagtacatagtaattatccttgtcggtgtaatagttaccggcacttccggccgatctgacctgcgcgatactcatcatcagaaatcatctcccggctcaacatcctccccacgctcccggtgcacattaatgttcacctcttcacgacgctgcatctgctgctggatgtccggatgattttcctgttgccatgcctcataagcggccatatccaccacttcaccggattcactgatcccgggtggcagttgctgttccatctcttcttccggtttcattttcagctcctgctcgatcccccctgccggaatattcacacctgaatctgacttcttatcgttgatggcaggagacaccggctgttgcggctgttgcggctgttgcggctgttgcggctgttgcggctgttcagcctgagtcacgtcttcttcggaaacaacttccggcacatccggttcgaagaggctggccatctgacgaccttctgcttcccttgcggcaagtacggcactcagacggttctccatttccgggttgatgtcacgcggaataaactcaggggcaaccttcggacgtgcctgatatttcagtgagagttttactgccggatacggtccgggcagggtgacataacaggtcagatccggcagagactgaatgtcggaataactgaccagcgtctggcgctccatatccttaccggtcgataccccgtcacgcacggggtcagcaccgtaggaatactgcagactggctttcaggtgctctttctcaccaatctcaccggcagcgaactctgcaatcttatggctgggagaacggaaaaaggcacgggtgttcatgacgtcaaacagcgtggcagccgctttctcaccgtagatatcttccagctgggcataggactggataccaaacacataacagccaccgaacttacgggcttccggcaggatctccaccaggtccggcagtttgtgtaacgtgggtaactcgtcacagaaaaaccacacacgacggttacgattttctcccattgccagcagaccacgaatggcaatggacagccacatggagatcaccggtttcagggaggcatgggtgtcggcattcgacgaaataaacagccagccgtttttctgatcttcccggacaccccgcatccagtcacggatggtgaagggctcaccgttatgctcaatcccctgcaggtaacggatagctttcacgtagttggtcagcacagcacggatggaaattgccgttttctcaattttctcttccaccaggttggccgccggtgaattacgcaggaaggtacgcagtttttcaattttgatggaaagcagtgtgtccaccagtttgctgtagctgcggttggggtcattacgcatcaggtacgccgcttccgcaaaaatggtacgtcctgaaccctgccagaacgggtcttcttttgttcccatagggatcagagtatttgcggtattatcaaaatccggctgtgtcaggcactccttccacaaatcccatgcggcacagcgggcatccagcggattcaggattttatcgatggagggatcatagtaacttttaacaaattcccctgaacggtcataaatcaccaccatatcaccgcgctgacgggcgtagttggccagacgacggataacctccgacttaccggccccgaccgtgccgtgcagacagaagttctggatttcggaatcccggataatcggcaggtcgccaatccggatatcggagtccttgccgtcttttttcagcatccgggcaacgtctttcggattgtctgtcagctgacgaccacctgtgacttcattttcgctctgttgtttaccctgacgccccagaatccaggagacaacaaagaaggtgatcaggcaaataaccagggccacgacagacgccagaacgaatgcagaccatagctgctcaccgcaccagatcatgtatttatcatgcagtacctgagccgcgttcatccggaaggttttgccgtaatactggatctcatatactggctgggacttgattaaatcccgcatgccttccagcgtggtacaccaccagtaaatacagccgttcacaaacgtctgccagcttatttttacccataaaaccagaccaacgagtatccagaaaaaaataaacaggcagtaaagcatgatattggcgatctggctgaacatgcggatacgcatggacgcaatctgaccgccctgggtcatatcctttgcgttaaaactcatgatgatattccgaagaaaaaaagtcagtcggactgattattaatgatgaaaagattaataaccgttaaattccggcttcgccggcaacacttcacggtcgcacaaagaatttgcacaaccgctcagtgttttcatatttatttcggaaacgccggggagaaataaaatttattttatttctttccggggttggacaacgagcaaagcgaggcgtcagtcaggaggccggtcaggccagcctccggaagataatcagagaatatttgcgattgatttggccagctggtcttcgagaacaggcttcgcctcttcaaatttcaggttaaccttgttcgcatttgagaccacacgggtctggtatttatgctggttacctgtttcagtactggtctgaattttcgcaccggatgtgccctgacgcagggcggcaacattatccgttgtcaccgttgccttagtacgctctgcaatctgcacatccgtgatcatggtatagttcacatcttccaccatcgcatctgcagccatacccaccagaccggcagcaaggcccacaccgagtgtggcaccggcagaatttgagttatatccggtaataccggcacctaacgctgcaccaacagctgcgccttcataaccacggttcagccatccctgagactcccgcagatccatcttatcggccttcagcacatttgcctgaatccagtagtaggctttatccggagaagtcaccacctgatatccttttgctttcacagcatcagcaattttgccctgcagcccactcatgtctttatcagacgtgtttttgatctgcagaaataccgtgcgttcgctgtcgggtttaagccagatggtttcactcatctgagtcttcacctcaaggttacgcttcttgattgctgtgctcatcgcaccacaccctgaaagggccagagtggaactgaccagtgcaaccatcattaatttttttgttttcatcaaattacctttattacaaaccacagttttcttatcgcctgtcatcatagtcctgccaacaagtcccaaaagtatttcctgctgaatcaaatcctccatgcatagctaatcctgttgccggattagtacgataaaaatccacataatcaaaattcacatcatcaaaatccagatgcaaatcttcattagtttgttcatttacctgagattgcatccattctcttacacttacacaaccactaattcgccttaattcgtatagtgtaccaatgataaaaaccatcaggcttaataacggagagagtaaggaagataatggcatcagtatataccaaacgcctttactgaatttaaactcatccgacacatgccgtttttttcgtatttgagaaaacaaccatataaacaatccctgccatagtacaataaagcacaggataaaaactgtttcgtcactcccccaatcagccagataacaggcaatgaactgtatcaccgtcaccagagcaatatgagctaaatttttcattatcatctccccatacgatattattgcttgtgcttatcctgatactcctttgctcttttcatcagttcctgtggactgtcagcaccaggcattctttcctgacccgatttttcttcattatatttattattctgagataatgcctctgttttatggtgattttgtagttcagaatactgtcttcctacaatattttcttcgccacgtatgctgctctgagtatctccgatattgccacgatattctgtgaccatattatcaacctgatgttttacatcattacggatattactgtcctgcgttctttgatcaataatggcctgatgcccctgatggtcagcaataatatcctggctgccaccacccgaaggtacgctctccattcctttaccaatatccccacgggattcacgccaggcattatcaacaccaggctgcacctgttcctgaacaaaagaccacgccatagcccgacggcgttctgcaatctccggcgaactggtatttgtcagaatagcttctgcatcctgcggtgcgcgtttcatcacatactgtgcaaactgttgcgacaggtcttcactcatctgcccgctcatgctttcagtacgggaggccatttccgcatattcatggctgcgggtcatattcgtcgtgtactggtcgtaactctgtttcgctgagttcagggctgcagacagctggtccacacgggaatcggcattattgtccgtatggctgccggattcactgaccttgcgactggtaaagtaatcgctggcctctttaaagtcttgtgtagccctggcatcgatatcatggcgagcatcatggctggcccgtgaaccactgctggcctgatgcgcatcctcatcactccagtcaatacttgcccgaccaccacctctaacacctgcgccaccaccaacaccaagaattttaggtttaaccccccattcagcatgaacatcgccatacatccccgcagaacctctcgtacttcttgatgccagttctcttgttgcctgctcattactgatattatgcgccttcgcatagctttccactgcactgcgcatacggctggccatcatggagtcctgtgcactcatggtgctgtcagcgccaccggtgacagaatcactgctcccccggttagaaccaaactgactgagcgagttccaggcactggcaatactgctgctgaacccatgcagggcactttctgctctgttcgatgcttcccgggccatttcctgttgtgccgctgcaatctgacgcgttgcattgataccgaccggtaaacgggacatcgctccgcttgcatccatcaccatattaccgtcacgggtctgtgtggcagttgcgccactgccggtctggtacatcatctgaccaaacgacgtggtgctgttggtgctccagctgaagccgttcacgttttccgtctgcatgttgccgtaggagtaattaccgtcaaccacactgccagctgcactggccgtcggactgatagaggaagaggcgaagtggctgtacacgctggaaaaacctgcgccaaggccttttaccatcatccatgacaacggggggatcatggctgaaagatacccggcagtagaggccaggtcagagtatttcagctgtatctgagagagttcagacagcacgaccggcgcaccattcatcttcgcatagaatgtcatggcactgttcagaatggcatacaacaacggccaggtctgcagccacatcagggcaaacacatagcctttcaggacggacagtgtcagtttgttgaagacagctgccagtaccagaagcgggaaaataccaatggcaattcccgtcaggatagtctgtaccattggcaggtttcgcattgtcacatgaccgatagagacatgcgccagacgttgcttctccatcgatgacgtggtggccagattcaccaggctggcggtgtcaccattacgggcggcattactggtgataccctctttcagggcattcatggtgacgttctggcgcataatctggctggcagactgactggagccgtagaaataactgtaactgtcactgaccagttgtctgaataacaggtccgggtccggtctgccgccaaatatctgctgcacataataatgccaggtcttgcctcccgtctttgtatcgagattcagcctgtctttcagcgtgaccgacgcatccttacaggttatgaaattattattgttgtcataaacgccccgtagcgggctgggtcgggaaaaaatcagcgtgtaggggtcagcggaggccatcagatcttccagcgtgtatttatgattcaggtaaatatcacccagcacacagttctggacgtagtcctggaacagattgatgatttccggattccgggacaggaaatcggtgcttttcacaatcagattcgccccgaacagcatgcctgtcttgctgtaggtgacgctgtccggttgggtgaatatcatctcgtagctgaccaccatcgcatgcccgatacgggtcgtaagagaaagtggcatcgccagaccgaccggaacattatccacccgatggactttgaccaggtcactgttatcaataatctgcaccgatgtgcggacattaaccagcaggctgataagcacaaacacggccacccagcccagcaaatccatcacgttatgccgctgtacccacataaaagccacagcaacaacagacagggtgagcgcgattttttcaattgaatcccaggtccgggtattcataaatgctgcaatggcattcaggttgttacgcaaccactcaccaccggcaatcacataaacttcattcacagtgaattccctccgaagtgatagttgttctggtaacggctgagcatacgggcggaaagctgctgacgcatgtagctcatctgacgttcgacaaccagcagcgcatcctgctggacctgtacctgactctggaaaatggcaatctgttgctgtgcctgattcagattctccagaacattgtccatcacagcctgcggatagttcccggtggcaatcatggcccgggcctgctgaagcagttcctgaatgtactggagcatgatgtcgtagccgatatagtccgtcagctggtaaacgacgctgttggacacaccgagcatctgcgggtcaatcagatatttaaaaacgggaatggtggtgctggaaataaacccctgttcccgggcgtcaagcggagtgtcactcactgctttattctgaatactggtgagcagttttgtaatctgtgacttcagggctttatcccgggcgatggtcacatttgcatccgccaccaccttcagacacttgtccgaatcgttgcagtggtaaatttttgccgttcccccctccataatcgcccggaggatatcgcggtcagtggtacgcgccggcaggggggtaatctcgctgtcttccccaaaaatcagcgtaccggtgagcgtcatgataaattctttcaactctttgttgccgtcaaacattctgttttttttcagcgcctcccatatgatattctggtttttcagcacccgctccttgtccttgtcgctggctttgtcacgaacagatgaggactgccctccgaccgtgcacccctgacgagatgcggcccagtcggcgaagatattgctttcactgccaatgtcctgacaaactttctgctgagattcctgcgtctgagggaacagcccccccacaagcccctgtgccacctgacaggaactgaggttcatactgttaatgtcacttgccattttctgcaggaagtcttttgcggtttttatttccggcaccgttgtctgcagggcaaggtcaaaaaagtaaccggcagcattgctcataatctgcttaacaaaacgctgcaactgatcgccattaataaaactgaacgagccgagataggcgtcgatgccgccacatccggcattgatatccggcagcgtcatggaaatcagctggatatttttgacctgggtgcgggcatacagggagccaccataggcatacccggcggcctgcccctgccagacgcccgcctgggtgacattggacgcaaatcccagcttattaaagaactgattcatgtcactgttcacatccgctgacgctgcgggtgtgaccgtcagtaaaacggcacaaagaaccagaagaggtttaatgcgaggcatcgttattcccctccctgaaactgttctcccggcgcatacggcgtttcagccggaatatcctgatccccccgtttattaattcatttaaagggttcctgacgtatacgataagaaatccacctgcaaacgcccataacgtatacggaatgacatacatcaggatgttccacatataacgaaaattatttaattccgggtcagctttccacgaatcaatacaggcatcaaaaatactgtgcataatcacccataaaaaagttccggcagcaacgaacacaaaaaacagatcaaaatatatatcagaaaccctgcgttcataatcataattccgtttagtttttctgttttttagttccgcatcatttcttttttctttcattataagtttcctctgatatttctgatgattcattcagacaggttatgcggctgtaatatgacaccgaaatccatcccacaattgcccccacaaaaaacacaacagaaacagaaaaggtaagacacagcgaaacgacaatttccacagggctggcgggataaaaaagcccctgacggataaaggggatattcaggctgacgattattcccgtcgcgatgccggatattccggcaatgagtaacaccacctgcttatttcgcacctttttcctctccgtatatctgcagaacggtatccatccgcgccataaaactggcggcatccgttgccccctgcaaaagcggtaatgcctcaagtgtgttgacgttgaccaggaaggtggtcggtgtggccaccgggatattcgggaagaaggtctgcatcacgtccggtggcaccggtaatgcttccggaaaggcagtatccccctgaccatccagggtgtagggaaaaacggaaaagccgtactgctgtgccagctgcttcagtaccgggtcaaactggtgacagtaagggcaacgcccctgcataaacagcaccaccttccagtcggccaggttaacctgcctgccgttgctgaggcggaaccagcggggagccggcttttctgttctggctgatgtgtctgctgccggttgtgcaggctgcacggccataccctgcggattccagagtcgttcaatttcatcccgggtggaggcctgcacacctgtggccatcatcaacaaaatgaccggcagtaatttagtgagagacatgtccgccctcctgctgcccttccatcatggcctgttgcctggcttccagtgcctcacgctcttccaggctgacacgtgcaatccatgcatccagcacctggtatgcgccgaacagcacaaggatggccgcaatcaactccgccagaaaaaaagccatccagggtttgctgtaaacaagacgggcgacgatatgaaaccagacacccagggaaaatacccacatacccgtaatatcaagccggggtaaagagaatctgcatttacttatcatgttaaactccggttattatttacatgagaatattcatgtctgtaatattcgcgtttgtgtgatattaatcagatcaacctcattattttgtatggtttttattaccgttttatatgactcagcctgtcataagccccttataaaaaaccacatcactccggaccatgctgatgacaacataatacagataaatattcttctgaacctgtcgaagctcgtaacgttcggtaatgagccaatagccattggaacaaccattgctgcgccctgattcataattgcaccacacagaaaccagagactggcggtaagcacatataaacacgcgatattaataacaaggaaaaccagtgaactcaggagaaacattttcctgtcattacgctcagaaaagaaataaccggcaataatgacaaatattaacagaacaacacctgtaaacattttcaaataatcttcactcataaatcaaatccgcgattaaaattttggtttaaaatcttcagaaacgttcaggaattgttttgccaggtcgtcctgcgaaataaagccgtatgataacgggcgaacactgccctgtttcgggtcaaccagcatcatggccgggaaatatttcacgccgaggcgctgcgcctgcccctggtcagtccgggaatccggcaacagcggattaatcacgccatccacggaaacgggaataacactcagaccatacgtatcccggaagccattaatgacctgcgccagctgcccgtcgatggggtcctgcccccggtaaaaaaacatgatgccgtagtgttcagccagtttcgcaatggcctgtcgctgctgcgcctggtctgccgccagctggttccgtaccgtgccgttgtaatggctgtactgcaggttatagtccagttcgggatgtgccagcatggcctttttagcactcatggtgaaaagcccggcctgctgagtccagtaattctgcagccggaaatatttcacaaaattatccacgccgggatacagaatggcttcgtacagcgcccgctttgttgccgtctgcagcgcggccagtttctgcataatatccggctcctgacggggggctgcaggtacaggtttattttctttttcctttggacttattttctcgttataccactgccagcctgcatctttccctgacgccggaaaaataaagcagcagagtaacagtggcagtaatgctttattcatcttcattcctttcatcattttttctttcatcagctttctgcaggccagtcagggcagaaaaacaaaaatacagaataaacaggagcacaccggagtaaaggataacgaaagcagcggcaagcatcgcgtctgccgtggtttcttttaccgtatcccacgaccagataattaccggagaaatgacaatcgctgtaatcaaaagccggataagaaaatcaacaagacgattagaagtgaatatgactttcatcatttcccccggttattttccatttcttactgtcctgcctgtttcagctggtcagcaatctgctctttcactttctgcgtcagaacgccactgtccgggattttctggttattcatcaggtcttcgtagaagttagtgaaatccagtctgtcgaactgaattttctgcagctcatcaaccgtaatcccccgacagtcaggatgcttcgcactgccaaaactgatacgcaactgcccgttacgcccctgctgctggactatctgcgccagcttcgaatcaaactggcagtagctgcgctttttctgcagacagacacccagcactttccgggagcagaactcgcccacactcaccgtcagtttgttgtcctttgcctttcccagcgccttttcttcactgttgcacttcgccagccccacatcctgaccccagccgccatccttacagcagttactgaagccggctgcggccttacggcagaatttcgcctcacccgtgaaagccctgacatccacaccattcagggcggcaacatccttacctgcggcggcaagcgcagccagctgtgatactgcttcaccaaaatcactgctttttccgctctgtgccttatcgcattcaccgtcaaggcagaagacatcaccaccacagaccatcacttttcctgatgtttttgactcgcaggagtacgtggcgtactcatgcagacaggctccctcatcggaataaaacgcgcactgacgtgaggccagcgtacaggccggattatcgacatatttctgacaggtgccgttatctgcggactgagtgacatatttatcccggtatgcccagcatgcctgtgtgatgttccagggtttaccttccatcactccggttttcgtaccacccggctctgaacactcagtgcctgtcagcacgccatccgctttattaaacggacaactctccacccactccacccggggaacccattctttatcacggaccttcatcctgagtttcagcgtaaaggtggaagcaccactgacaagcgattcatagactttcctgtcaccattcccgtgcgggaggcaattaccgttagcagtacagccactaccggtcagaacctgcccctgtgtcacctgcaaacctgtcgccccggacagggggtaagtatccgactgcccgtttgccaggccgacattaaaggttgtattcatgaaggtataacgggagttcaggaagaaaaaagcggcgtatacagacaatgatgcactttccactgtaccggttaccggagccgtgacactgaacaccagtttcagaccgttcatactgaaccggaactgtgagtgcggtatcgtcacctcacgatattcatccgtgtatttccagtccccggtgatactggcagtccgtgtgcagtactcctccaccatcgtgtcccgctcacaggtatagtttgtgaactcactgcggtttatctgctgcgcactgcactgctgcccggtgtttcccacaatgctgtcagcccggttcaccacatccctgccggtctggataaacggtgcatccggtgaaagaatgtctttcggcttgttcatgaatgactctgtgatagtttttccggtttcaccggtcgcccattccgtggtgccgtcatttttcaggccgccatccccgccggcagtcacgccaccgtaatattttgtctcgtccggattcgcgttatagccggggatactctcctgtggcttaaagccctgaatactgcctgtaccctgcccctgaatctgacgggcaaaatcagaacccgcccggtaatcactgttgctgtcagcctgtgccacactgactgccagccccagcagcaatggcagaagacgtttcatacctcactctccctctgatattgctcaaaccagaacacgactggtttgttcaccacctgaatcagcccgaaccgttctgcggtcctgaaattcacgctgcttctgcgcccccgttcaacctgcagggacacctgcgttctgaacatctccagcgaatatgttgttttcagcaggttgcacggcgcacaggccgggacgaggttttccggacagtcggcctccggtctgaaaacatcaccggtggatttcagcctgaaaatgcgcttttctgcagccttcatgcactgctctgacttgcggagcaccgcctgaacatggtcggcatgccatcctttctccggcagttcacagccgcagtacgcacaacgaccgccgaatttcatacgcagtttttcccgctgtttctttgtcagcgccatcaatgcctccttacgggaacagatttacggattcagtaagcggaccagcgtcctgaagtctgcacgcagtgagcgacgacagtaccggtgataccagtgatgagggccttcaggctgaagccggaaacagagctgaaggtcactgttaaaagaagccccgacggaccagccccacagcaaccggggcatcatggactccgcatcctgccgcagtcgctgctcaagaagagccgccttttttaccggacagacaaaggcaatataaggcggcagtttcgtaacagggaaaccatgcccctgacaggacgcatatgtacaaaatccgtgaacgttcatcgcggccaccagtgcacggatgtccgggtcaatccggtaatagcctgcaggtaaactcattgcgacttatccccggtgccgtccagcagtccctgagccacctgccggcagtctcctgcggtggccactttttccagcgcctgcctgacgcgcagatttccccggatgatgtcgtgcccctggctgcagaacaccaccagggccggaacgctgcgaatgccgtactgtgaaaacagcgtcgggtcgatctgaacaccatcagttgcgccgtctttcaccagggacaacacggcgtcagccgtggccttcaggtcgttattcaccatgccccgcagcgtggcgggaataccgtagtgccgggtttcgcccagcattcgtttcagcccctcttcgggaatggaaaacgacacaaaatacagggcaccctgccggggttttccgtcctgcgaagcctgctgtttacggaccagttcatcgagaaactggttatcggaatgctgaaggggattcgccctgacttgctgctctgcccatgcctgcaactgtgtgtccggcttttcacgtaactgctgactcagtgtttcctgctgcttcaggaactggcggttttcagttgtgttcatattttctgaagcatgaacagagataaacagtcctgctgtcagggcgaataaaatgtatatttttttgttcatataattcttctgcaaacaggcagtaattaatcactacaaccaatatctgatatcaacaaccagcttctcacggtatttcagggattattaatcccgggattacgcgcccggtcgcgttgtggtaaaatatttcagcctgatatatcgattgcaagagatgacaggctaaaaacataccaacaaggagttacaatgaataataacacatcagaagaccgccctgatttttcattcagttacatggacaatcagagagcagatgaaataaaggctttaaaaatattattattcagcatcatcagaaatatgcctcatgatacagccatcagtattattaataacatcgtaaatactgataataatgaggcgctcaatagattatgcctggagataactgacgccatgttaaacacaacgagaatcgttgattagcttctttccccatttatctttttcctgagactggctgcaattaattgccagtctctgtattgctctgctgttattacctttttctccatactgaaagtgtcacggggtacagggagagatacatcctgatgcaaggggagctgcaggccacattgtttctgttctttttccatcatttatccttttcttgtcagtgttataaaaatacacagttacgttttcgcgtagttgtctgcttaaattttcctgctgcttcaggaattgtcagttttcaggagtgttcacattgtctgaggcatgaacagtgatatgcatccctgcagccagggcaaaaagtacaaatattttccggctcatatcatctcccgacacagtgatataatttaattaccggcgcacttttctttcagtctctcagacaatgcatataattcatctatttcttcctgagttcagcccattctctgaccacatcaaaagcactcaccctttccagcagggagaaaaaacgctcctcactgataaaaatttctttttctttattacttatctttcgtaaaaatgataaaaaactcataacacacttttatttatattttacatatgcacacacggttatatttatgagaaccgacagactcatttcctgcgcttaacagacataaacttatctgacgaaagagacaagacaacaacgcttaccactgtcagaataagaaccgtcttcagatacagtttgacaagcagtattttttccagtgtatctgcatcctgccaggcgttaacaatgtctgagataatatgttcagggcaaagataaaaccacaccacagggcatatcaccatagcaaaaggaaacagttttaatacattgcctgtcaaacgaatgaacactcgggatatactcatatacatatgaatgtatttactgcttaatttcttttccatttaaaatattcctctttttgtttaagttacagaaatacacagttacgtttacgccacatcaggtagccgaagtttttctttgtgttgggcggattttttcccgtctcccagcgcatcacactgcgcccgaacggatggcactgcccgctgtccggatacatattcaccatctgataacgccagcgttctttgggcaggattggggacggatattcattacagacggcgttatttttcccgatggtttccataatcatgccctgacggtgtagtttgaacgccatgcgttcactgaccagcagggaggactgcaacggactggactcattactcacccagccattgaacgggtacatacttccctgcgaaccggcacaccagaacagaacatcgagaggcatattaaaggcgctggcaatggcatctgctgcacaggccccctgtgctatcggattggcaaagatgacagcttccggattgagaatggtggtcaggctgctgtccgtccaggtggggtcaatttcagaaagataagcgatatccaggtcaccaccttccagacagcccagcgatgtgatgatgttcagccagtacgtcagcgggtatttgtaccagtgaacgtgatagaacgccccgttcacctgcttctcatcctttttcgccgttccctgtgccgttttaccaaaagccggcaggctgaagcccaggttaaccatgcatcccggtgaccgggtgacgtccgtcagcgccatcggctcccagtaaccaatggccagcccgatacgcctgaacagcggcggcggtgccggacaaatctgaatgggcatcgacgggttcgccgtgtcggggaccttaccctgactgactttgatactgcccagcgagagcgggaaaatacagctccagcagatatccgtgatcgggtttacaaaacgcccctcacaggcagaatccgcagatgccgcagggacatgaccggcgagaaggaataacatcagcagccacaggcttcgcttcattttctgccctcctctgccggaataaattccaccttcaggaagcgatcgccgggaacggcgctcacccgtgccgggacctgatcaatgccgaggcgctggcagagcacaccgttctggtcaaagtaaatacggctgtccagcgctttctgcatctccgggatactgccctgcacgaggataattttgctctccagtgtgggcggcgtctggcgtttcatccaggccacctgcgccggatcatcgccattgatgaaatacagcgtctggttaaaaggaacatactgcagcggattcatcacttcaccctgacgggcaaacacccgcccttcgttatcccggatatccgcagccagtctgacggacgggtcaaacaaccggctgctgtatttctccgtgcgtccgataccgggcacggcagggggccgcagactgttacggatcacccgctccttaaaggcatccattttccggcccatctcaccggactgctccagtgccgtcaggcgctgcatgatcaccgtcagcatgtccggctcctttaccggccacagatcgccccaggtgccaagatcggcggcggccacactctgcccccatatcagcagggcaatcagcccccggcatctcatggttccgccctcattcgccgggcaatatcctgctggatttcccgggtgatatccggtgccccctgtaccacggcaggcgacaccagaatgaccgcgtggtgtttctgctgccatgcctgcaggctggcttccagtgccgtgttaaaacgcgctgaaagggcttttgactgtgcttctgacagttgtttctgactggcgctgtcaaaaaacgcatccacggtctgtttcatgttgaaggcggcagtcaccggtgcgttcagtctgacaatgccataactgatggctgcgttcagtaacaccattgccaggcatcccggcacggtccaccaccagtggctgcgtcggggggctgtaacgtcagtcgttttttgcgttgtggtcatgcaacactcctgtatttctcatgttcttccagccaggcctccagcgaagccatctccggccccgatttcttccacgccagctgccacaccgcctcatgaatactcataccctctctgcgtttctgctgcacaaactcaaaatccgggccgtcagagctgtacatggcacggcttaacgggtccacaaacagacggtgccaggaggaatggttttccacctgcagcaggaaggaactgaaccactggtctttggccgcaccaaacttaccaatcatgtcgcgctgcagtggctggaactgatccgggaataactggttatatttcgcgaactccttcgcactctgtttgaggataattttgtaggaggagttaccccatgccgcacgggctgcgctggatgccttgtcagagtcaaagtcgacgatgttctgtgtgatggtgatataggcaccggtatgacggcgacaggtgcggtaacctttttgaataaactcaccaactttacgatttttgaagtccagcagacgccagccttcatcaatgacgttcagtttcttgagggtacgcggcgtgcggtacatccggttctcgatgtaaatgataaggctgaacatcaccgcaaccagcagtgacggacggtcttccagtccgcccagctccagcaccaccatttttgcgtcatcccgcagggacggctcatcagagttaaaataccggccgtaagtgccgttggcagtgtactggtcaagcagcacaatcatttcgtccagacggctgcggatggtcggtgactcgacatactggtcgttatcacgtgcgtttttcaggaaatccaccacatcatcaatgcgcgcctgcttctttttagccagccaggaggctctgaccgcctgcagcaacagaccttcatgcacttcatccaggttaccgttggggctggccatcaccgacaactggtcacggacacgctccgctgactggtcaatatcagtgatgttcgcaaacgggttaaagcgcagggtttcaccgtccagatatacgccccccatgttctcacacaaagacttgtagccatcccccatgtcgaacaccacggcaaagcccccggagtccagcacgctgcggataagtggctgtatcaacccggttttaccggccccggatgttccacagaccgccatgttgtagttggtgttattcatccccttaaagaagatatcgataaacgccagctggttacggtaggtgggtgccagcaggcctgccggtgtcagggggttatccgccactaacggcatcaggttggccacattaaagctctctgctcgctgaaccacaccggcctctttcagctgtttaaacagccctttcccggccataaagggcaggcaggtcaggaaattgcgcatgtggttaaagcgcggtgaaatcagctcaaaaccgtttttacgaaagctgttcagaatgtcctgctccacttccagcgccgtttcattgttgtccttgcagaatgctgtgatgttaaggaagtaggacacgacagaagactgaccggagcccagccgctggcgcagttccccccactccttagcttctttctccacggagggaaaccatttggcataggaggtcttcgactttttctccaggtccatgtacttcaggttggcttcactgtgggttttcacctgatcttccaccaccagtgtcagcgtcaggatgaacgggcaggaaatggacatttccgggttcagcagattgctgtagttatcggccatgttccacaggaaggcgatttccgggttacgggccagatggaaattcaggatacgggctgtgctgttcctgccgttctcacgcagccccagcgtcaggtaatccgcccggaccttcagatcaaaactgtcctccacacactgataattcagatcagaatacgggtccaactgacgtcttttcgggtacagagagtccggattatggttaatcatctccccgacaatctcgatgaaagcctgtgcatccactgtctgtgtggtgatttttgcgccgtgaaaagaggcacggatgattttcaccagattttccatttccagaatgtcggcccggctttttttcttcgagggagaacagtacgagataaacacccggtaatgacgcagggtcaggggcagattcatcccctccggcagcgggaactgtgtcgctgccgctttcatataataggcacgggtaatggcgttaaaccgttcagcctgttcacctgaccaggagaactcacgcagcccgtattcaatcctgtcaccaaccagctgactggacatcagatggatacacaacggaataccgcgcggcagtttggtgcgcagcatatgatcgagcgcctccacaatggactcattcgcaccattgatgggaatggcttccagcataaagcccatcgtggtgtcattcatgaacaggccggactcctggttgtaatcacggtacggcagcagacggctgaactgcgggaagctcatttcgcccagaacttcattggcctttgcggattcgtcaggcagtttcagtgctgtgacgagggagttaaccgcctgagtgacggcctcaagtgggttattcacatgaattccttagttgaaaaataaagcgagaaatgcggtagccatgaagggaatacctgtaattaacgcgccatttcccggcgcatatacaaagatttcggttttctgaatatcaactcatgttcatccggaattaaatcaatatgttctgcgactgacttatgcataatgttttctctgcctttcctgatacgcctgacattcaacacataacgtcacaccgggaaatattttccgccgggcttccggaataggattaccgcatgcttcacagagataaacaggaataccatgagcatttattttctggcgtatccggtttattcctgtcatggtcagttgttctgttactgaatatgcttcatcgacttcatcactcacggaatattcctcccgacatgttctgacacgctaccgcccatcccttttcacgcacagggcactcgttttacaggcagattttgaatacagaaagcccgccggcattgccggctgatatacaacttcagggccagttaattaatacgtggttttccccacgcagacggtttaataacaaaaaacacactggatggctgatgataaacatcctgattatcaatataaggcgctatccataatgcagccgtctgctcacccgttcttagcgggcggggaggtgttaccggcgcaactgaactgaccggaacaatcgttttcacttcccgggcagccgtaaacaacggacgtggtgccagcagtttctgctccggaagtgccgtcaccgcaggtctgctgccggacggagtggtcgcggtgatggtctgcaccggcattgtccggaaattcccttcagccaggcgcggcagtgatgccgcaaccggctttgcttctgaggactgctccagttttttggccttctcattggcctgctccatcgtcatacaggtatcggatgtggtggcgttacactcaaattccgtactggttccggcacagccggaaagtaataaggtccccagcagaggaataaataaagaaatctgtttcattgtccctctcctttctcatcgcagttcacaaaatcattcttcaaatccctgaccgtacaggtttttacaatgtcaggattttcaaaaaccgtattccgtgatgatttataaacatatgccacaatgcgtttcccccccgcatcaggttctggtgcactgacttcacataacggaggtgttattccggattcatacagcaccggataatttatttttttgacgctgccatcagaaaccagcttattcatcgaagggttccttatatttctgaataattctgtcgattagttctttcaccggaacagattttccgccaccggattgtccggcaaccacaatatgccccggagaagaaaataagctcatcgacattcccttcttcatcacaccgacagcagccatattaaaattgtgcggcaatgattcagaactgaattccttcggtgcaggaagtacgattcttgtggtgcagttactgagaacagcatgcataaaatcatctgatacgcttttatccggtacagagcgggcagtaataacattaatattacgcatgttcatttttatctccgttgtttctgtttgttctgtcatcaggaaaataaacagggcatttcctgattttttccggcagggaagcgtcttccgtataaacttccgtcagacgttcatctgcggttaccccccggatacatttcatttttccgggcagcaccggatatttcccggagataactgccagtgagacatacatcaatgccagtgttgcccataatgcggtaaccagttgcatcagtgaccggtcagtctccctgacataatctgcagcatcagcagcacggtaacggcagattttctcttctgctcctggaagatcatgcagagaaggcaaacactctcttacaggaaactgcccggcaggggctgtttgtcttacgaagtcaatttcctgaaccagtaactgccctggcgtggtgttgtccttccataaaacaaacagagccatcagggccattgtggccagtggcagaatgacggtgtacttcagacaccagaacagtcctcgcagaacccgggcgacaacataccgggcggcatggcacgcctgacgggacgacatattatttgccatcgttgccccctccctggctgccggacataaactgctgcggattcaggttgcccagctgcttcagcatctggtcagtattaaagccgttaagatgactttcagccgatggcggaacaggaaccggactttcctgattctcttcttccgctctgctctgcgtgcgttccagcgccatctcttccacggttttcagctggaagccgtcctggaacaccacggtcacttcgttgcccgcaccaatcggtatcaccgggtgatactgttcggcacgtttgatgtagtagtcactgagcgtctgtgcggctttcgatgcgccgccaccgatacccattttcaggacatcaccagccccgtaagcggctgtggcacccagcccgacagccggctgggaggcgcgctccattccctgaccgataccgtcaacaaatcccgcgccccatgcccagccgaggattttgccgttacgcatcaccacttcgcccttgataccgtttttaccccggaagctgacatgccccttaatcggcatatcaatggttttgccgtccttcaggcagctgatattgcgggtgcgtacaatggcacgttcactggacacatccccccaggcttccagccccacaaaacatcccgttgcgtcatacgttttgctgttcggcatttccaccagaccggtgatacgcagctgcatcggcaccgtggattcattaccggtgactgaggcattggcatccgctccttcaatcagcatggctttcgcaaaacttcctgacggaatgtacggcagcgatggtccctgttttccctcattacgggtaaacaccttacgctgtatccggttaggcaccggcacagactggtacgtcacctgtggtggtggcgtgacaccattcccgggataaaacgccgtcggcggtggaacagcaacgctgccctgcggcgggaaggatacgggggtgtttcctggctgtggttcgccttccggcccgggcggtgaggctggcgtctgcggtacaggctcaccggtcgcggtgacgggatttgcacccagggcttttacctgctctgccagggcagcattatcctgtcccagcttttcaatacgacgctggtcatcaccgcgctgtttgttcaggacatccagttcacgacggatttcctcatactgcttctgcatctgcgctgccgtcacctgcatctctgtggtggcatgctgacgcaccttgtcatcaaaggtcgtatccaccacaccggtcatatccggcacaggctcctgttcagccaccgcctcaccgttaccggccatgtccacatcagacagatacagtgcaccaccaatcgcggaggctgcaccgacaaccacaatccccagccacaggtactgcttgcgtttcacaatggtattgatactggccatcagttgccctccccgtcacggatgacatacacatccatgcgcgcacctgccagtaactggctggcaggctggctgaacatcacggcacgggttcccggctgccagaaatcactttcccgaaggttattcaatgggcagacagcgttttattttccacggcaaagcggaccatcttcaggtgattccccgtccatacggcatctgccactgaagacagccccgccggggcctgcagggtttcctttgtcacaggaacctgataccagcctgcaggtaattttccgccacggacagcctggctgatggtcacaagcagggattcgtaaggcgtggacgtttcccacgcgccggcttcttctccggttccgcgcaggtcactgaccagctgaatggtacgccccgcaccttcacggggaacggcctgaatggaaaggttcagaccacgttctgtttccagaatgaacgtaaagggctttttgttgacggtggcaaccaccacaccgccggaggcggtctgctcattattggtcagggcaccatccagactgttcacggcgataatccggtcaccgggaacggtaaacagattcgggctggtattgctgatggcaatactggcctgaccaccgttcaccatggcacacacgtgggggccagcgtaccgtttgcggccatcacgctgcangaaaaaaacagctgccgaatattattgcccgtatatttttctcatcgtctgtttcccgaaattctcagcagtcccccgttttcccgctcagaataggaagtaatgttggattcaggaaaggtttgatccccatccggttttaagccccccagaatttcacacggcatcggaggcatacccggccctgttggaaaaagctgagttcagttatcgttttaatacgcttcgcttcttcagccagattaatttcatctggttctgtgcgcccgggcggatatattgcagaagcgcttgatgtgaggcatcgacggtttctgatgaaacattcagacggagggcaataaatgacagcgccatctgctgtaaataagaggcgtcggcactgttctgtgacacggcaaagggcacattaaatcccattggggtcacggcagtgcgttgctcattctgaagacggtagttattcaccccctgaatgacgttaacagagaggctgagaacaataagcactgacataaatataaaggcgatggccattacacgactggtacttaaacgggcaccgtgttccatataacaatcctggtatcagttctatttaatccactgccggaaacacgaatcgggaacattatgaaaaataccgcgcagcagggctgttggcatataccagtaaatcaggtcacgtaaccagtaactgcctcgcccttttttcagttttttaatcccgaaataaaccagaaccgctgcaccaataccgaacagatatttcgatgttgtgataccccagccaatacagattgcggcggggatcagttcatccagtggcagggcaaaccagcggctctggttggtcagtgtttccgggaaacgatatttcttaagtttattctcgtctcccgacatcgttttatttcctgtcagaggccaacgacggccatacccacagcaataaatacagagatgatggcaaaaccggccaggaacttgacgtttttggtcatcatgtacatgaccgcaccgaccagaacttcagccagaacaacccatttaacaacactggagtccttaccgaaggtcgccttaaccgtggagttaccactctgcatcaggtcctgacctgccccctgggcggccagtgccagttgcgggaagaacatcatcagaacagcagcagggatcacagcgcgagccaggcgaagcatattcagacgagtgaatttagaaaaaacgatttctttttgacgggcgcagaagcaccctgaacacttaaaacagtattcatatgattcctctgtttaatattgatagataaaaactaaagttctttaaatgttgactcgttctcttcgatcacctccctgaatatttcttcattatagaaatcaggaaaccgtttcagatgatctctgagacggatctgaacctctatagttttacttctaccgctacgatctttggcccgaataagtcggttatttgtatcttcatcgagatacactgagactgttctgcttatcccgcctctcgcgttacgcctcctcactcaattcacctctgtttcccgactaataaaatcatgattgcacagaaaccgacgaaaaaaaacacgccaaaattggcgtgccttatttaacatttacttaacaccataaaattcacgaattatacttaatagcgaatctatttctcttttatagatacatgacactctgaataaatcaaagtgttcaaaaaattcttttattttccgtttaactggtttaattcgatcctcagatgctctgtctgtaatacccaactctctggcaattgttgcgtgaccaacacccagcatataaaaagagaaaacaaccaatgtccctggagaaagatcattccatatcgttaatttatcagaagtatttgtattacttccactctgatttaacgcaaaaaatggataatcaggaaacggattaatttgccataaaacaacagattcgtcattataaaaagaaacattctccatccttatctgatagatttcgccatgaaaattaaaacgacgacaaaatgcagagccatgtccaagagcctggcattccagttcaagtgatgaaagaaaaatttcaacctgcagacgtatataactctctccggataagggtttatcttctctggaaaagagttcaataaaagccccattggcataaaggattttccggtttttgttacggacacaaaccggaacgggagaatttctgtagatattctgaacgttaaggatgtatttatttacagattgactgttaagtggcctttctctacgatccagcgcacacataggaacctccgttaaggattctatggacagtcgatgcagggagttcagacctccctgcatcggcgattttcagtttcaatttttaacgtggcattaaccacgtaattaatataacctttatgcggttaattgtcatcaaattgaaccagatcaaaatcctgttatttgaagcaaatcaacttttaattcttttttattcctcatcattttctggaaagaacctctccatctcagatgacaccttatccctgatatcttcgaccatattcgcatattcaaactttggattcccggaaacatgagggcttagtgactcaatacctaaaatcttagcaactgttgactgtgtttttaccgcacattcaagaagtaccttattaaactcagcctgattgaacgctgactctttacgctccatctgagcctcatatacacgaagccctaattcgagaagcatagtggatatgcttgagaaacttacatctgtacttttagctccttctgcacgtcttctttccacaattttatttattttatagacaatttcgtcactgacataagcctgcactttcgccatatgtatacctctttattttcttatcgcatcaaatttatttcacatataaaaaaatgacaattcatcgatgaatcgaatcgtgacgcaagttacgaaaattacgattcatgcagcaaatcacatcacgaattgaatctagagtcaattcgctttaactcgttgttttattattactgattcatctatgagttgcgttaaattcattggtgaatcatatgcgattcaccaatgaatcctttttataatgtaaaataaattaaaatacattatttaaaacataagttaatgattcaaatagcaaatcagcaaaaacttgtttttgcgtggggtgtggtgcttttggtggtgagaaccaccaacctgttgagcctttttgtggagtgggttaaattatttacggataaagtcaccagaggtggaaaaatgaaaaaatggatgttagcaatctgcctgatgtttataaatgggatctgcgaagccgccgattgctttgatcttgcaggtcgggattacaaaatagacccggatttactgagagcgatatcatggaaagaatcccgttaccgggttaatgccattggtattaatccggtaacgggatatggcagcggactgatgcaggtagattcccagcattttaacgaactggcccgctatggaattaagccggaacatctgacaacagatccctgcatgaacatttataccggtgcttattatctggcaatagcctttaaaaaatggggagtctcctgggaggccgttggtgcatacaatgccggattcaggaagaccgaacgccagaaccagagacgtcttgcctacgcatcagaggtttaccggatttataccgggataaaaagcagtaaaggcatccgggttccgaccacgaagaaatcactttcccaaattaacagtgtgcagaacaattaatattgtatacattgtcaaataccggttatgccgcgacagcggcatggaaggccacacacaaaaatatcatatgagtgataatgctgactggccgaagccccgcaaagcgggaccccatcttatatatatctggtaactacgcagcctcctgccagaccatttttcgcgggcacaatatttacagactaccgaaaagccagttgatgaaattgaggcatatcagacgacacagcaatgccgcaggatgataatataagtaaagccccgtaaaaattttcggggctttacttatattatcatgacttccactccctgaactgtccggcaatcatccataatgctttgttgattcggatatcaccgtcaatgcctgtaatggctcttgttctggtatttttaccggaggcacttcgccccgataatccaccttttatcatattctcctgaacccgctgccaggttgtccagagatcgttctgtttatcctcccaacggcggggagtaattatttgttctggcgtcactggcgttttattttcatcttcgtacctggccatcaatgcagctctgccaaataaatgttgttcgtcactgttaagatgtatctctttcattgcctccatattttcagtgactttatcaaagacaccgagcacttcatacgctccctcaataacctgcccgacaatatcacctttatgtggaacgcggatttcgccaaaattattcccacacaccaggccatttgtgcagacaaaacgaaaaattcctgggatcatctgataactggatgaaccatcatgtgaattaagcaggataatttcaggaacttctttcccgttaatatgcccttccctgcgaagacgtaacatatgtttactgtattcgcggcgtcctaaatcacgaacccgactctgacaggcaaagaacggctggaaaccttcatcacgtaacttattgatgatattgatggttgggatatatgtataacgctcactccgggattcatgtttatcaccagaaaagacagatggaacaaactgcattaattcgtcatccgttaacgggcgctcacggcgaatagaattatatcgaccaaaacgggaagctaatcgcataacagtaactcctttatgaaggcataaaattaataaaagttttttcgcttaatatcatccccctcggttatcgttaacaaaaagacaacaattccgaggggaaggaccatcaaatcaggtcagttcgggactgaccaacctgccgagtgaaaagcagtaacagcgcgatcaactcatcagcgttttttctgacaagaggagtatgattctccaggctatccagtaagtcatgcagctcattattgacctggtggagcaccttaaagagtgcaccagaatcccggcagctctgatatgcaagatttgcctgaacagcacgattctgcatgatcccgatttgtgcgattgcgtctttctgctgatcgcctgttggctgataagtggtgttaaaagaaaatgcgctcataattcctcctgtatcgatgttttcagcccccatcatctgttgagtttcgggagccgcggttttgacgttttgcggggacctgccttttgttccctccggtattcaccatcacctgtttgaaaaccttcgccgcaaggggcatcactgccacggcctgacgcacctgtcctggggagaaagcaaaaatttttaccgggttttgggtgaaaaaatttgtgggctgttcatttaccctggacagtcagtgcggcaggttgtggctgtctatgcccccggcgacaggggcttaaacaggtgaatggcaccggacagaacaacgggcagggacccgccgcaacgacgtgaagcgcggtttgcgaaacggcgttgcgccttacatcccggcggccttttgccgggatgccgggagcgcaggccacggcgcgacagaggcggcgcgaccggaacggtcgctgcaagcccggggttttcacgcatcggaccgtagcgacagcccgaaaccgcgccagcgggttcggcggagactggcgggtgcgccagcagctgcctgtcggagtcgggcctgacgaccggatgccgccaggcagccgggagcgaaggtgcgcccggcagacaggacaacactgcagtagtcagaatcagagcggcaggaaaaagaagagcagtagcagtagcagtagcagtagcagtagcaggaacaacaacatcatcagtaacagacaaatgacaggcatgaaaagacagaagcacacagcagaacatccgcgtaatgaaaggggcggcgcgtaagcgcggcccctttcagcgccaccggcgtgctgtcttttcccggcgcgggcagacgggcatttatcctcccgcctgcccggcgtcttcaggcccgcagacaaataatcatctcggggcacgggtataagcaaacatgccgcgcgacagctccccgggaatgcagaagacgccagcaacgtcttctgctggcgtgatttgttcagacctctgtcgaaccgatccatataccattcagataagcatcaaatgcatcattcccctcctgcacttcctgcggctgaacaaggttcaggctttcgtgtgtccagccggcttcaggtgcagacactttcagcagaagatgcccatcagaatcacaaatctccagatcagcaaacgtgttcccggtcaggatatctggttcaccgggtcgttgatctgtaacatatgtgatatatcccatccattctctccacattaaatatttcagcactatccgggacgacggtgcggcccgattttcagtattccagagcaagttcagccatcagagcatctttcgcttcccgtgccagcctggtttcttccatcgtcagaaaaagcccctttcgtgcagcagcaccaatgaataccggacgatccttcagaggaagccgggaaagatgattcatcgccatatcaaacaaattcgcttcataaatttcatagtgcagaccacatccccaatgagcctgcttttccagcctgctgatcaccacatcagcccggataacatcaaagggattgcgggaatgaatattacgaataaagccatcggtacgtgctgctgacatatttctgcccatctttaatacacctctgttttttgcccccatcatcttttgagtttcgggagccgcggttttgacgttttgcggggacctgccttttattccctccggtattcaccatcacctttttgagaaccttcgccgcaaggggcatcactgacacgacttgacgcacctgtcctgggcagaacgcaaaattttttaccgggttttaggtaaaaaattttgtgggctgttcatttaccctggacagtcagtgcggcaggttgtggctgtctatgcccccggcgacaggggcttaaacaggtgaatggcaccggacagaacaacgggcagggacccgccgcaacgacgtgaagcgcggtttgcgaaacggcgttgcgccttacatcccggcggccttttgccgggatgcagggagcgcaggccacggcgcgacagaggcggcgcgaccggaacggtcgctgcaagcccggggtttcgcgtcagggatggaagcccgccagggggacgcccggaaaagacacagccaggagaacaacaaaacagacaataatcagggcagcagtaatcagagtcagaacaggaggaaaaagagaataatcaaagggaagggatgtgatttctgacaggtcataaacagagaaggcgggggaaaagaacgggcgccgcaggcgcgcgttcttccccgttcccggagggaaacggccttcctggcagtctggttgttcatgcccgcacaatgccagcaaggagaaaggggccaccggcgaaccagcagcccctttataaaggcgcttcagtgcagtcagaccagcatcagccctgaaaaggcgggcctgcgcccgcctccaggttgctacttaccgccggattcgtaagccattgttgccgtaacttccctgtatccgtccttcagccggacttcgcacaggcgattccgggtcaggagtgtgaatatcaacaacgtgcagcacacgattaatacgcaccagataagggcgtttcccggtagtttcatggtttactcctccttgcaatttgcacggtaagaggccatactggtgttggttagacatacagtgggcctcgtgggttaatgaaaattaactacggggctttctgctttctgccacacaacacggtaacaaaccaccttcacgtcatgaggcaaaaagcctcaagcgccgggcacatcatagcccatatgtctgcaccctgtcagcatctgcaccgttctccgtcacgccccggatttccagccgacgcagagcactgaacagataatcgcattcagcctgtttgtttgccctgaacggtttcgcgaccgtaaccacccactgatgatttccgggccagccatgtactttcctgacccgcccgttaatcgcatacagcaacccccagcccgccggtaaatcggccacggtaatcacccccggctcgcagagataaaaacgccagtctcccatgcccttttccggctctgaccggaacacctttttccggtcagccaggaaatcagaccgtgagcatttcacctcaatgagacatgacgctccgttacggaagcccagggcatcaggatgttcaccgcaggacgtccatgcgtgaaaacggtcactgaacaccaccttaaagccgttgttgcggagaaaacggcaggccagttcacaaagttccctgtgtgttttcgggtgtctggcgttctgcatcacgccacctcccggagtttgttcgggaccatccaccgctccatctcacggacagcgtgaccggcctcccttgccgccataccagcctctgcgatgacctgccagaatggcatatcctgccactgccagcgttccatcccggtcagtgacggaaaacgctcgccccactgccagaacagggtttccaggtcatgctctgccacccggtcactccagcgtgcatgccagcttccgactgtttccggcgaatggaatgccagttcgatttcagcctgagcgaccagcgcctggtacgcacgggctttctgctctgcctccctcgcctgctgacggctgtacttctgcatgaccagtgcggatttatggcgactgcgttccctgacacgaaaccgcaccgacggaaacaaccgctctgctgcatcaccgggaaggggaaggaggcagacctcgccctgtgtttcaatcagcacatcgacggcatacagccactgaagacggttgccgtgcagttcttctgcggtcagggacggcatgaaaaatcgcaggtcagacagcgatatcctgcggcttccgttcagacagcggaaaaacgcccccgcatacggatattccgccagcgtgttaccctgatggcggcgtttttctgtctccgccaccgcctgccatgcggcctgttgctctgtgcagagaggaaccagtgcctgtgaacgtacgctcatgcctgacttccttccgccatcaggatatggatgatgtcattaacgctgtacccctgcgcatccagtgacgccgccagtgccagtgaatggctgacagcttccggtgcaaaccgccgctgcacctgaaccaccgcaaacggctcaccaccggcattcaccagaaccagcacccagacctgagagacatcgcccggtgaacataacgccaggtgaaaacgttcgtgggcaggcgtgaaacggacctggacggggaaaaatccgccaaactcgctgccgtactcgccgttcatcgtccagttgtccggcgcatccagttcccgcatcacggcgtgtgtcagctcacgacgttcatcacttccggcagcccggatatcttcatattcctgtgcgctcatggtctgtaatacattcagggtcagttcagttttcatcacagtatctcccttgctgccgccccgtctccggcggggcggtgtggttgttcatcaggcggcatcagccaggttatcagtgcgttcagtggtatctgtctgtggatgtggtgcacacatccagccaggcacccagcggttgtctttcatatggtgctctgcatgttcagccgcatcacctttcttcatcttctctgcatcccgtgcggcaccggacagtccggcatcattcagggctgcgataatctgcggctttttcaggtgtccgaagaagtttgcttttgtcggctgccaccagtctcgcatgtgaaagccgatggcggtttccagcgtgtcaagcggactgcgtgacgtgtgaccacactcacgggtctggacaccgttaaggctgcatgcggtgcagaaactgagcagggataacagcacctcctgactgagtgacaggaatgtcgtcatgtcccgtgaccatccctccggtaacagggcagcaagacgggctttttctgccatcagcgccatgaatgcggcaccttccttccccgatggcgcatcgctggtcagcgaataatgtttacattccaggctgatttgtgctggtttactgtacgctccgctgccaaacacattcaggcagagcgtccatgccagcagtgtcagggatttgtccggttgctgcatcagtgctgcctggactgccagcgtgcgttcggaagacattttcgtcagcagtggcagactgatttcctccactgacgctttctcctgcacctgttccgtacggtcagcgtcgtcagccacgtcatcctcactgcgcaactgcacaccacgctggacacacacgttgccataacgccaggacaccaccactccacctccggcacgcatctccggcgtccacgccctgaccttcgccatgcagcgcatcagcttcatttctgcttccagcaggtcagattcctcacactggttttccagcgcgtcgtaacgcgtcatcagttcgttcagacgttcttcttccgcctccgtcagcaccgcttccggctccggcagacagcggtatgttccggcatcctcacggcactcaccgacaggctccatgcgtccggcgcaccattcccagccttcggcttcccgaaggaactctgcgacagcctggagtttttccagcagggcggcatcgagcgccacgcagtccacatagccaccttcgtcgtcgctgaacaaatcggtgcgcagttcgtctggcgagaaggcatcagcccccacgaagcggaatttactgttccccgccaccgccacttcactttcggtcaccagacgacgaatggtctgtacttccggtttaccgccccatcccgactgacaggcggcttcaaacacctgcacctgacgcgcggtgtcgttctccagcgccagcgcctgacagtgttcggtggtgatgcggtcttctgccagcgcatcgaggatgacaggcgcaaggtcagccagtttcagcattcgctgaacgtggcggggtgaatagcccagcaaatcaccgatttgtgcaggtgttttgccttcctgcgccattgcgcggaatccggcaatctgttcggcagggtgcatatcccgacgctgaccgttttcagtcattgatgcagccgtcgccagctcctgcggaataaccttcacgcgtacaggccagtcagccggaaggatgccacgctctgccagcatgttgagtgctgccagtcggcgaccacctgcggcgacaccgtggcggtcaccaggcagggcatgaacaaccagattctgcaacaggccaacgcccttaatggactccgccagttcgctgacggactctgcggaatacggcaccgtgcgcacattcagcggtgatttaatcagcgaggccagcgacacgctcacttcctccgtctgctccagcagggcagacaggactgtttcctgcgtttttgcaggtttacggctggatttacgctccgtttttgtcttagactctgtaactgacatatatttctcctttgaccgggatggttgtatgtctgccggacgcgttcccgtccggcacctctcttacttaagaatgccgaaattccggatgacatggttcatccagtacccggcaccgtcctcaaaattccaggtacgccagaccaggccactgttattgcggacaaccaggcgaaaatgcgttccctgatcatcctcgataaagacattccggtactgtgcggcaacggcttcagcctgttcgcgggtaaaggagccgtcaggcagcccctgaaatattctgaaatattcgctcatcctcatatctccggtgatgccccgcactggacggggcggttgtcactgtctttcgttagaacggaatgtcgtcatcgaagtcatacgcgggttcagacggctgctgaggctccggctgcgcggccttacgtccacggcctttcgttttcgcgccaccttttttcggttccggctgtgattccggctgaggctgaccgctgaactgttgcgcctcttccggctgagtctgagtacctgcggcacgtcccagcatctgcatggtgcccgtggtcttaacaagaatttcagtgacgtaacgggtgataccgttatcttcccagctacgggtacggagttgcccctcgatgtagacctgcgcgcccttgcgcagatattcaccagccacttccgcgagcttgccgaacagcaccacgcgatgccattctgtctgctcccgcatctcccccgtctgtttgtcacgccagctttctgacgtggccacctgcaggtttgccactgcgcccccgtttgggatgtaacggacttccggatcttttcccagacgaccgacaagaatgaccttgttaatgccacgaactgccatgatgtgtctccttctgttgatgttttcagcccccatcatctgttgagtttcgggagccgcggttttgacgttttgcggggacctgccttttgttccctccggtattcaccatcacctgtttgagaaccttcgccgcaaggggcatcactgccacggcctgacgcacctgtcctgggcagaacgcaaaattttttaccgggttttgggtaaaaaattttgtgggctgttcatttaccctggacagtcagtgcggcaggttgtggctgtctatgcccccggcgacaggggcttaaacaggtgaatggcaccggacagaacaacgggcagggacccgccgcaacgacgtgaagcgcggtttgcgaaacggcgttgcgccttacatcccggcggccttttgccgggatgcagggagcgcaggccacggcgcgacagaggcggcgcgaccggaacggtcgctgcaagcccggggttttcacgcatcggaccgtagcgacagcccgaaaccgcgccagcgggttcggcggagactggcgggtgcgccagcagctgcctgtcggagtcgggcctgacgaccggatgccgccaggcagccgggagccgaaggtgcgcccggcagacaggacaacactgcagtagtcagaatcagagcggcaggaaaaagaagagcagtagcagtagcagtagcagtagcagtagcagtagcagtagcaggaacaacaacatcatcagtaacagacaaatgacaggcatgaaaagacagaagcacacagcagaacatccgcgtaatgaaaggggcggcgcgtaagcgcggcccctttcagcgccaccggcgtgctgtcttttcccggcgcggacagacgggcatttatcctcccgccagccctgtgtcatcaggcacgcaaacgaataatcatcgtcgggacatcggtataagcaaacgtgccgcgcggcagctcctcgcggatgtcagaaaacggtaacagcttctcctgctggcgcggcccgttcagacagacggcaaccagcacgccccccggacgcaacagggaaaaagcccgcaggatatgccggatatcctgcccgtggatgaacggcggattcataatgatccggctgtaatactgcaccggctgccattccatgaagtcgccacactgtaccctgacaccgttaaaattttcccgcagataccggaccagcccgctgttaatctccaccgcatcacacatggcctccggcgcggtatcccgaatcgcccgcagtatggccccggtgccggcagagggttccagaatgtgatcacggttgctgatttccgccagcgtcaccaggcgatcacagaccggggcaggagtcacaaaaagctgagattcagcccggatttcagcaaccggcgcggcgttatccagccttgcgcgcatcagccggatacgttctcttaactgactgtgcatgtcattatctccgtcatcccccggggatttcccggggggatataccttatttctgtgggatttccgtgattttcatgtcggtaatatacacattcaccaggcggaaattattgtccattgtgcggcggtagcggtatgccccgtggtcttctgtctctgccacactgcgcacggccttacagtcccggggcagggcggcccactgcgcctttgtcatttcccggaaaccgtcccccggatagttgactaccggcggacgcttcgcggcctgactggcgacggcagcctcttctgccgatggtgctttgtagtccgtgatgcgatcgggcgtcactttcatcgtgccgctgtacccgagaaaactgtaattcggcgttgtgactgaactcaccgccccgttgcttttgttcacgcggatgatggtcagccactcgccccggctgaaaacctgtccgcccggctcaaaatcctgtgtccgggtaaccacgccgccgctttcgtccagcatggcacgctcatagatcaggcggttctgatagtggttaacccagcgttgctgatgctgaatctgccgttcatgacagcggatcgcaatttcgcgggcctgctccgtggtgatgatgtcatcatccagggccgaccataacgatcgacttccttcatactggcttttttctgccgggcgcggatacctgtccagcgggaaacaggcgctgatatggtcatgactgctgatgagttttgccatattcagatccagcgattcagcccgccacatcgtcagatatttctgcgactgtgcgatggtcttttccgccttgcgcaaatcagcttcgatttttttgatacgacgccagcgaacgtccggacgttctttatacttcgcgtgaagcagtgccgaccgcgcccgctcttcccagtattccgcacgttcaaagagcatcacagcacgtttcataccgttttcaatacgctgtgcatcacgacgggcgcggcgttcgctgtgatgccccacaagaatgggctgaccgggcgggatcatcgcggccagtctttccacttcatcaagtgcctgtgcggattcactggcccgctttccgctgtatccggtaaaccgctccgcccgtgcttcctgacgttcagcgagcgtgctgtcttcatcctcaatctctccggcaagtgagaggagcacgtcttcccggcccggtgtccaggctggcgcgacaaacagcgcctgtttgggtgcccagcgaaaaccggcatcatgcacttttttatacgtctccgggtcaagacgtgacgcggcatacaggcgcagtttgttgtcatccggtgagtaggtcgcgcggtacatctgcacagggctggcggcttccggggcggtcatggtgattgctgctaatgtcataaaatgttctccttccgtctgttgccgcccctcgcggggcggctcacctcatcagttaaaagcgcgtttcagttcgagtgtgcggagcacctgcccgaaggccgtgtttttcatggcggggatgtgtacaccacgcccccggtgaaaactccgggctatgtgtcgcccgaaggctttcagcacggggtcaatttccggcgcggggatgtcttcgcgtctgaaacggggatgacggcggggctttacagccgctgcggaaggtgaaacaatacttattgtcataatttagcctcttatgacgattgactggagccgcaaaaactgttcgtgcagtctctgcggcatggattaagcaggttcctgacctgcttttttcatatctgcatctgacgctttccggtgttttcctccttctgttcctgcgttctcattacctgccagccggatgactgacagaacacataaatgtgtattgccggagcctgaaataaatcctgatttttaaagaacatcacccgttgagtttcgggagccgcggttttgacgtttcgcgggaacctgctgctgtattccctgtggtatgaacttcacccgtttgagaaccttcgccgcaaggggcatcactgccacggcctgacgcacctgtcctgggcagaacgcaaaattttttaccgggttttgggtaaaaaattttgtgggctgttcatttaccctggacagtcagtgcggcaggttgtggctgtctatgcccccggcgacagggactcatacgggtgtgtggcacccggcaggcaatgagcaggggcccgctgcaacgacgaagagcgcgtgcgcgaaacggcgttgcgccttacattccggcggccttttgccgggatgccgggagcgcaggccacggcgcgacagaggcggcgcgaccggaacggtcgctgcaagccgggggttttcacgcatcggaccgtagcgacagcccgaaaccgcgccagcgggttcggcggagactggcgggtgcgccagcagctgcctgtcggagtcgggcctgacgaccggatgccgccaggcagccgggagccgaaggtgcgtccggcagacagaacaacactgcagtagtcagaatcagagcagcagataaaggaacaacaacaacatcaggaacagttccataacataagaaagaaacactcctgagtcagacacatcacagacgggaagggtaacgggcgacgcgccagcgtggcccgcttcccttccggcaggcgtcagcctgccaggggttttcgcgcgggctgacaggcggcctccaccgcctgcccctgtatcacctgtttctgctgccctgtaagatgtagtgaccgcccaggaaaaccgcctgctgattcgccacaaaatcgcagatttcatcaacaatctgcgcggtggtgagcggtttctgccccatttcacgacgccgcgcattaatcacggccctgtaattacgcagaatttcgcgggtttccgtgctgatattcatgacatcgtctccccgtccagcgcctgcaacacaacgtccattgcttcgcgttcctccctgacgaatgcgtcaccgtcgcccccgttctcactcccgacggcatacaggcgcttcatttcactgcctgccagcgccatcactttttccagcatcaccgccgggactgtcacctgacggctggcgtgcttttcctccatcagttcacgaacaacgtcgtgaacaacgctgacatccgcaccatgctcaaaggcatcgtccagccgctgcatgacggtttccagctcatcgtctgtcagactccagtcctgggctatgtgacgcacatccccacgggccagcaccacggcaaaaacgcattcactgtccggcacgtctgtatccagcacttcgcggataatttctttaacagtacagtacatgttctcgtctcctttacaggggcggcattgccgccccgtccgttaatcagtccagcgccgcataaatcgcgttacattcggggtgaaattcgatgtggctccacaactgcgcatcgcgcatcctgtacaggtgagtcagccctgcgctaccgctgtcgtggtacagccacaactggcggttaatcacaagggaggtcaggatgatgcctgcggcatcagcactcacggtacggtcaaaccagttttcaccgttaaccagatgaaaacggtcgccatccggcatcatgtaaccgccaccgtccggcaggcggatgaagtgccagaatccgccttcatattccggcatcatttttttcgccagcgcgtacacagatgcctcggcatacagaaaatccaggccaaacaggtaaggcagaaaggagagacggtcgcactcttcaacgttcagagtgacaggtttcgcatattgcatgatgttttcctccgttgttcacatattccgcgacaccgggcggtgccgtgcctccatctttgccgggcctgcagggttgcaagggcgcaagggaagccgcagcctgacgcacacggctggcgggagttggagggtgagaccgcgaaatctgagcgggtgaacgaccggaaaatcacggcagacgggaaggagggaccgggtgaccgcagcggttcaccttaccttgcaacccgcccgcagaagcccggctacgatggaggcacggcacagcccgcacacgacgctgaggccacataactgaaaacccacaaggccatgccccgcatggccgggtaaagcggagcctgcggcgacatccttttgctctttccggggaccggctggaaaaggcggagatttttccccgccagcgtcatcatttccggattttcgccagtgcatccgccatgctgcggatttcatcccgtgcctccagaacaacctcccgcagaatatcccggtcaaaccagcgacgggacggttcatccggtatacgcagcacatcacagaccagaccactggacaggatatgctccacacactcactgaccagatacatttccaccgccgaaaaacgtgctccccggttcaggatagcatcgagcaccacgggcagattttcgtcatcttcatcaagccagcaggggtaatcgctttccttcacaaccagacgaaagcggcgcgtctggcgggtcagcaacgtctgaccggtggtggtcagcgccgcacggatacgccgcgcatcttctgccacccggcaactgacactgcatactgcatcgcgcaccgcatcaagaaccgccctgatttcttcctccgtcagccattcaccggcatacgggaaagacacctgcgttccgtctgccggaagtaccggcagcgactgccactgactaccggacagaacaagttccgctccggttccggtcactttctgaatggcagaggccaggagcggcggcagtacgccgccgtgacggttgataagtaacgtcgccagccggggcgcatccagccgcacgtaaatgctgatccagcccggatagtgcggattcagttgttcaatatccgtctcttccggccagccctgcgccagcagcaggttacgggcgtggcgataaaccagtgcattcagcgttttgttcatgattaatggtctccctgaatcgccgcccgaaagcggcgataacatcagtcgttttcgtgtgtgttgtttttcagcagggcgcgggcctgattaaccaccgcctgacggcgggtgacgcattccgggacggcgaggccatgatcccgggcggcccattcacgtttccacagtgcctgttcggcggtctgaatcagggacgccagcagttcagccgggacggtcacctggcggttttctctcacgttattgatgatctccatcgccacgccggaagaaataccggattcagtccgctggtcttccgggatgtcctccaggcgcgccaggacagtcctgatttcatgatcggacagggaaatatccattccgtcagcgagggcctgaatttcttccggggaccagataaccagcatcagcggcgtgtctcccggatactgcgctgccagctcccggcatagcgtttcgcatgttccgtacattctcgtctccttctcaatctttgaagcaggaatccagttccagaatttcggtatacgcctccatgaacaggcgattggcgcgatacagcgtctggcgtgtcaggcgttcaacaagcacatcggaatcaaagcgacagcgatgctccatatcgttcagcctggcccgaaggtcacacatggtgttggccacctccgccacgtcggcgcgaagtttttcgtgtccggcatagttcatttacgcagcctccggttcaaaccagttatcattcgcggccccctgctgcatggcccgttgcacggcggccagtcgttgctgtccggcacggtgatactgttcaagcagctcgataccgatataccggcgtccggactgaagggcggcgacgcaggttgagccgctgcctgcaaacgggtccagcacaatcgcgttcgggtgtgtgaagctctcaatcagcggttgcaggctggtaacgggcttttcagtcgggtgatgacgattgcctgaatatttccagcccagcacgtcgggtaatggcttctgaggcagcgccggacgtccttttgccaggatgtaggcgcattcgtggcgatagcccacatatgcggccttcgatgtgtaggttttggtaaacaccaggtggccaaccacgctgaatcccgcatttttccaggcggtcataaagcgatcgacgcggttccagccatagaagctcaccattaacgcgtcttttttgagtacgcggtacatttcattacaggctggttgcagccattcatcggttttatcgccggcgatggtgcgcccggaacggtcacggaaaccgacgagatacggcgggtcggtgaggatgaaatcgacagcattgcccggaaacgtggccataacacgcacgcagtcacccaggataaaacgggacataacagcactccttctcttacagagggcaccgggagggcacccctgtggtgcccgttccccgctgcctgtgggctgttcaccgcgtccggtaacgggtcaggccgaccgtggaataccgcagccgtcgtctttttgacggcaaggctatgccgcgaaagcggcttgaagggcgttcatcagaaaagggctcccggctggcgtacactgtcaccaacgccagccgggagcggtaaaagcgcccgtcccgtgcccggccggacgcgggacactgaccacaggcagcgttcctgccgcgccgcgtcagccggcgcagtaaaaagaggagcccggcgacatcttttgtcacccgccaaacaaaacccaaaaacaacccacacccaacccaataaaacaccaaaacaagacaaataatcattgattgatggttaaaatggggtaaacttgacaaataaacccacttaaaacccaaaacatacccaaatacacaccaaaaaaacaccataaggagttttataaatgttggtattcattgatgacggttcaacaaacatcaaactacagtggcaggaaagcgacggaacaattaaacagcacattagcccgaacagcttcaaacgcgagtgggcagtctcttttggtgataaaaaggtctttaactacacactgaacggcgaacagtattcatttgatccaatcagcccggatgctgtagtcacaaccaatatcgcatggcaatacagcgacgttaatgtcgttgcggtgcatcacgccttactgaccagtggtctgccggtaagcgaagtggatattgtttgcacacttcctctgacagagtattacgacaggaataaccaacccaatacggaaaatattgagcgtaagaaagcaaacttccggaaaaaaattatattaaatggtggggatacattcacaataaaagatgtaaaagtcatgcctgaatctataccagcaggttatgaagctctacaagaactggatgagttagattctttattaattatagacctcgggggcaccacattagatatttctcaggtaatggggagattatcagggatcagtaaaatatacggagactcatctcttggtgtatctctggtcacatctgcagtaaaagatgcactttctcttgcgagaacaaaaggaagtagctatcttgctgacgatataattattcacagaaaagacaacaactatctgaagcaacgaattaatgatgagaacaaaatatcaatagtcaccgaagcaatgaatgaagcacttcgtaaacttgagcaacgtgtattaaatacgctcaatgaattttctggttatacgcatgttatggttataggcggtggcgcagaattaatatgcgatgcagtaaaaaaacacacacagattcgtgatgaacgttttttcaaaaccaataactctcaatatgatttagttaacggtatgtatctcataggtaattaataatggacaagcgcagaaccattgccttcaaactaaatccagatgtaaatcaaacagataaaattgtttgtgatacactggacagtatcccgcaaggggaacgcagccgccttaaccgggccgcactgacggcaggtctggccttatacagacaagatcccagggcccctttccttttatgtgaactgctgacgaaagaaaccacattttcagatatcgtgaatatattgagatcgctatttccaaaagagatggccgattttaattcttcaatagtcactcaatcctcttcacaacaagagcaaaaaagtgatgaagagaccaagaaaaatgcgatgaagctaataaattaattcaattattattgagttccctttatccactatcaggctggataaagggaacttaatcaagttatttcattagcaatgattacataaaataaatacagtgtcgttattataaaattatttcacggcacttataaaaaaacagtaccaataatcaaacctggtgagttcgcaatgtctggcaataacgtttttgatgggttgttttccagcccggagcgcgatattgccaccagtcacagaaacaccgagaacaccagatttcagcggtcgctgaatttgaacgagatcttttacttgaacgaacttattcagggatagcaagagcacgggatgcaggaaaacgttttggtcgcccaccggtattaaatgaagaacaaaaacagaaggtactcgaacgcattaagtctggggcaagtataagtgctattgccaggaaattccgaaccacacggcaaacaattctgagagttagagcgaaacagcaaactgttactgtatagaaaatagtcttagcgtgggattccgttttccaagcggaccccaaggaagactacatcgcgttcatgccgaaaccgaatgtaagaacggcattgcataatcttgcagtggcgatcgaacattacaatgaaaaccatccgcacagtgcgttgggttatcgctctccgcgagaatatcgacgtcagtgggtaacgttaacttaagatacaacccctgtctggaaataagggggcaagaacacattgacactgtaaatatggcattatataaaactccgccccaataatgagtttcatttgcattactcccatttgctggtcatccactcgcctgtttacaaggatagcgtttaatgaagaaggtcctttgcgcgttaggcttgatgttcaccgccgtcagttccgcgctggcaacaacctaccccctgacgattgaaaactgcggctatcaggagaccttcacccggccgccggagcgcgtggtcgcgctggggcaaaatacggtcgaaattctgctgctgctcgggctgcaaaagcaggtggtcgccagcgccttctggccgaccagcgttttaccgcagctggctgagcaaaacgcgaagattaaaaccttgacggtggaaattccgagcctggaatcggtgctggcgcaaaatcctgatttcgttcccgcccagttgccgctgctgctggggccggaaagcaaagtcgccaggcgtgaagatctcgccaccgtcggcgtgaacagctacgtctcaccggggatgtgcgccaccaaaaaggccaccggcgatatgtacggcagccggcaaaagctgtgggatatgacctggctgtatcaggaaattagcgatttttcccgcattttcaacgtcgaagatcgtggtcaggcgctgattgccgatttcaaaaaacgtgaagccgacctgcgccaggagtttggtaaaagcaaaaaggatctctccttcgtcttctggttctccagcgcctcaccttcggccgatgcctatgtcggcggcaaaaacagcgcctccggatttatcgccagcgtgctgggcggacataatgcgattacctccgaaaccgagtggcccacggtgagctgggagagcattattgccgccaacccggatgtgatcgtggtcgccagcctggatcgtaaccgctgggcgctggataaggccgaggaaaagatcaaattcctcaaaagcgatcccgccgtcagccagctggaggcggtgaaaaaaggccatatcgtggtcatggacggtcaggcgatgaatccgaccattcgcacgctttacggcgcggagcaggtgggcgagcagctgagaaaaatggggctgaactgatgagcgtcgccgcgattgagacgcgccgcagcatactgctcaccggctggtgtgtactggcagcgatcgtgctggcgctggtgatcgctgtgggcgtcagcgttggcgagctggccatcccgctgcagaatgtcttttatgctatcagcaacagaaccggcttaaccgctgagccgctcaaccgtatctatgagagcgttatctgggatttccgcctcagccgcgcgctggtcgccgcctgctgcggcgccggcctcgctatctgcggtgttgtgctgcaaagcttgctgaagaatgccctcgccgaaccctacgtcctcggcgtctccgccggggcctccaccggcgcggtgtcgatcgtggtgctgggccttggcgctggcgcaatttccctctcggcaggcgcctttgccggggccttcgccgccttcgcctttgtcgccttgctgactaacggcgcgcgcggcggcaatgagcgtacgattcttgcgggcgtcgccgcctcgcagttatttaacgccatcacggcctataccatcagcacctccgccagcgcgcagcaggcgcgcgatgtgatgttctggctgctgggcagcttcagcggcgtccgctggcccgaattccagttggtcatcgtggtggtgctcgcgggtctggcggtctgcctgtggtatgcccgggcgctggacgcgttcaccttcggcgacgatgccgccgcctcgctggggattgctgtgccgcgcgtgcgcctgatcctcttcaccactgcggcgctgatcaccgccaccatcgtcagcatggccggctcaatcggctttgttggcctggtggtgccgcacgtgatgcgtttcttctttggcccactgcaccgaactttgctgatcgccagcgcgctggcaggggcgatattgatggtactggcggacattgcgtcgcgcctgctgattgccccgcaaagtctgcccgttggggtagtaaccgccctggttggggtgcctttctttgccgtgattatctaccgctcaaggaataagtgatgagtatttgcgctgaaaatatcacctggaaggcaggcaaaaaggtcatcgtcaataatgtctcgctgcgggtgccgcgcggcgaaacggtcggactgctggggcccaacggctgcggcaaatcctcgctgctgcgcgttctggcgggcctgcgccgcccggatgcaggtcgcgtcaccctcgacggccaggatatcgcccggatggcgaaaaagcagctcgcccgccgcgtggcttacgtcgagcaacacggcatgaccgaggccaatatgcgggtgcgcgacgtcgtgcgcctgggacgcattccccaccactctccgttctcaaactggagcgctcaggatgacgaagcgattgccgccgcgctgcagcgggtagcgatgctggagaaaagcgaacagggatggttaagcctctccggcggcgagcggcagcgggtgcatatcgcccgcgcgctggcgcagagcccgagcgaaatcctgctggatgagccgaccaaccatctggatatacaccatcagatgcagttaatgcagttgatcagcgagctgccggtaaccagcattgtggccattcacgatcttaaccatgccgcgatgttctgcgattcgctgatcgtgatgcagcaggggcagatcctcgccagcgggacgccggaggagattttgtccgaagcgctgctgtgggacgttttccgggtgaaaaccaaaatcgagatctccccttaccacggcaaaaagcacattcatttcatcgtttaggttgggcggatatgcctcttttctccctgcgccctgcggcaaccctctggcctccggtgttactgggaagccagttcgtttttaatattggcttttacgcggtcgttcctttcctcgccctcttcctgcgcgacgacatgctgctctcaggcggacttatcggcctgatcctcgggctgcgtaccttctcccaacaggggatgtttatcctcggcgggacgctggccgaccgctatggcgctaaggcgatcattctcgccggctgcgtcgttcgcgtcgccggttttctcctgctggcgtgcggggcgtcgctgtggcccattatccttggcgcctgcctgaccggcgtcggcggcgccctgttctccccgtcgatagaggccctgctggcgcgggccggtacccacagccaggccaacgggaaacgcagccgcgccgagtggtttgcgctgtttgcggtatgcggtgaactgggagcggtgatcggcccggtggccggaggcgtgctgagcgggatcggctttcggcatatcgccctcgccggcgcgggaatatttcttctggcgctggcggtgctctttttcggtctgcccgccgacgggcacacgacaacaacgcgcagacgagtcccatggtggacgcctttgcgccagccgcgctttgtcgcgtttattctcgcctacagctcatggctgttgagctacaaccaactgtacctcgccctgccggtggagatccaacgttccggcggccgcgagcaggatctggcgccgctctttatgctggcctcgctgttgattatcaccctccagctccctctcgcccgcttcgcccggcgaatgggcgccgtgcgcatcctgccggtgggctttctgctgctgtcggcttcctttgccagcgtggcgttattcgccgccgccccacccgccgaaagttggctgcggttaatgccggcggctggttttgtcacgctgctcaccctcggccagatgctgttagtgcccgcagccaaggatctgatcccgctgttcgccgaggagtcgacgctgggtgcccactacggagcgctcgccactgcaggagggtgcgcggtgttagccgggaatttactgctcggtcatttacttgaccaggcgctcattccgtcgccgcaggcggtttatccctggcttctgctggcgctgtttccgctgtgcagcgccgtcgccctgcgggcgatctgtcgcccgctggcggcaacatgacgcagagcgggtaacagcccgctctggaagtgaacttcattgcttacggccttgaggccgtaccgtctggtgtgcgtgccattgtccaggacggtatccccccctcgcacggatatttcgccgctttggcttcatcaatgtcaatacccagacccggcttatcgttcagataggcataaccatgatcaatctcagggcagcccgggaagacatcacgcagcgcatcgttcattggcgtgtattcctgaatgccaaagttcggtgaactcagatccagatgcatattggcacagaccccgaccggagaaatgtctcccggcccgtgccatgcggtgcgcacgccgttcagttcgctgtagacagcaagtttcttcgccggcgtaatgccgccgatggtgctcacatgacagcgaatgtagtcgatgagcttgttgtcgatgagcggcttccactcattcacattcacaaacagctcacccatggatatcggcgtggaagactgctggcgcagcattttcagccagtcgatgttttccggtgccaccggatcttcaagataaaatagctgatactgttcgagcgttttggccaggttaatggcagtcacaggggtgacgcgctcgtgcacatcatgaataaattcgatgccaaagcccagcttgttacgcaggtgatcgaacaaacgcgggacgcttttggcgtaggcatccgggtcaaagtagatgccaggcgttttgctgcgcggcgaacgcttcggctggatgtttttcgcccgcgccagctgagtggcgatcagcttgagatcatcagtccccgcgccgccgtacatgcccatctggcatcgaacgtactgatagccctcttccattctggcgcggatattatcttccacctccacttcatcgccgccgtcggtgtggcagtacagtgggatcccatcacggcatttgccgcccagcaggtcataaaccggcatccccgccagcttacctttgatatcccacagtgccatatctacgccggacaacgcattgttcatgattgggccattacgccagtagccgcttaccgcgcccgactgccagatatcctcaatacgcgtcggatctttgccaatcagaaaaggtgccatgtactcatcgattgcgctttttaccgcgaaaatacgctgagtaaatgttgcacatcccagtccgtacagccccggctcgttggtctcgacctttacgaccgccaaatcaataccgcccggcgccgtcagaatcgttttcacgttggtgattttcaggttactcactttcactcctcaactcagcgaatttaccgtatgtcataccgttgtggatgacatgacacatgtttgtaagttgtcctacaacttaatgctgttatatcacgcttctttcgaagaggagcgctgcgagcgccattctgtgacagcgatcaaatctcgttcaatttaaattaaattaaacaatataattcaatgaattaattgaatttaagtgatcgccatgcggagctgtttgtgatccttgttgcaaataaatgggtgagttgttgattttatcagagcgaaaattcaaccttaaataagacagtcgtcatacaagttaaacgagtgaggaaacaacatgaccgctttatttgatttaaccgggaaaacggctctggtaacgggctctgcgcgtgggctgggctttgcctacgccgaggggcttgccgttgcgggcgcacgggtcatcctgaacgatatccgcgacacgctgttggccgaatccgtggatacgctgacccgtaaaggttacgacgcacacggcgtggcttttgacgttaccgatgaacaggcgattgaggcggcttttagcaaactcgatgcggaagggatcagtgttgatatcctgatcaataacgccggtattcagtaccgcaaaccgatggtcgagctggagctggaaaactggcagaaggtgatcgacaccaacctgaccagcgcatttttagtctcccgcgcggcggcaaaacggatgatcgcccgcaacagcggcggcaaaatcattaatatcggctcgctcaccagccaggctgcacgtccgaccgttgccccttatacggcggcaaaaggcggtatcaaaatgctcacctgctcgatggccgcagaatgggcgcagtttaatatccagaccaacgccattgggccgggctacatcctgaccgacatgaacaccgcacttatcgaagacaaacagttcgacgcctgggtgaaaagcagcaacccctcacagcgctggggtcgtccggatgagctcatcggcaccgccgtcttcttatcatccaaagcatcggattatatcaacgggcaaattatctacgtcgatggtggctggctcgccgtgttataagccgtccgtactctacctacttgcctttaaaattgacggcatgtgttcaccttgatgcatgccgatataacacaggtattattatgaaggcttcaagacaacgactctttattctcacattgctgtttcttgtgactgcaattaattatatggatcgcgccaatcttgccgttgctggttcgaatatccagaatgatttcagtctgacgccaacacaactgggtttgctgttctccatgtttacctgggcctacgctgccagtcaaattcctgtaggctatgtccttgatcgcattggctcacgtattctttacggtggcgcgattattctgtggagtatttttacctttatgatggggttcgcctcacatcatttattcgcgacggcaaccgcttcatttgtgatgcttctggcctgtcgcgcattaattggtgttgccgaagcgccgtccttcccgtctaatacaaaaataatcgccacctggttcccggaccatgaacgtgcccgtgcaaccgcgatttattccagtgcgcaatatcttggtctcgccctgctaaccccagccctcgcctttattgtggctaactacggttgggaaatgtcgttttatctgtccggcggcgtaggaattctgtttggcatctactggcttatgtattatcgcgatccccagcacagcaccgccgtgaaccaggccgaactggattacatcaaggcgggtggcggctacggctcggccaaccagtctgcggttagcgagaaaatcagctggcaagatattaaattcttcctcagtaaaaaaacgatttggggtctgttcatcactcagtttgcctgctcgtcgacgctctatttcttcctgacctggtttattgtttacctggaaaaagggctgcacctctctatttccaaagcagggatgggtgccatgcttccctatattatggcgatgcttggagtgctctgtggtggtacgttaagcgacatgctgctgaaaaaaggcaaatcccgcacgctggcacgaaaattaccggtgatggtaggcctgtgcgtcaccatgattattggcctggtgaacttctttgaaaaccagccggttattgcgattgtcattctgtctgttgcgttctttgctaacgccttttcaaacctgggttgggtggtctggagcgatgtgattcctcgtaatttcctcggtaccatgggcggatttttaaatatttgcggcaacctttcggggatcgttagcccaattgttattggggttattctccaacgcacgcaaaacttccagtatgccatgtggtatatcgcaggcgtcgccggactgggcttattagcctacatcttcctggtgggaaaaattgaagtgatcctgcctgaaaaaaagaatgccggcaccgtggataagaatgccattaatccggcaactgccaacaaataatgaggtcatcatcaaaatggaaaaaataacctgtaacgcttgtctggcccatgcggaaaaagacgtacgttttgaatcacgtgaaatcgagcatggtgaacatgacgtcgtcgtgaaggtcgcctgcggcggcatctgtggctctgatattcactactatcagcatggacgcgcggggatgtctgttctcaaacatccgatggtgattggccatgagtttgttggcgtgattaacaaagtgcccgcaggcagcgagttgaaagtgggccagacggttgcggtgaacccgtccagcccctgcaatcagtgtgagatgtgcctttccggccatcaaaacctgtgcggctccatgcgctttatgggcagcgcgcagttcaatcctcacgtgaacggcggcttttctgaatacgtggtggtaaaaccggagcaatgcattccctacgatagccgcgtacccgcaaacgtgatggccttttcggagcctctggcggtcgccattcatgcgataaaaaaggcgggccagttgaccggcaaacgcgtactggtgattggcgcaggccccattggttgcctgatcctcgcggcggcgcgcagtgccggggcatctgaactggtggcgtccgatctcagcccgcgctgcctggaactggcacgtcagatgggggctacggcagtgatggatccgcgcgatgaagaacaggtcgcccattatcagcagcataaaggctattttgatgtcgtgtttgaagcctccggcgcgccgattgccgtggcatccaccgtcgatttcacccgccctgccggcaccatcgtccaggtggggatgggcgccagcccggtaagctggccggtatcgccaatgctggttaaagagctcaactgggtcggttccttccgctttatcggtgagttcatcaccgcggtacgctggctggaagatgggcgcgtcgatcctcgcccccttatcagcgctgagttcccaccccagcaaattgaagatgcgttgattaccgccacagataaaaatatctctgctaaggtactcattcgtttcgattaacaatgagaagcgcctggccgggcgctttgttcttaaaagtgaattgttatagaataaatcacttcagcgacatcttaacggatacccattttgagcataaaatccattcaaaaacagaatgttgtaaatgaaatttatgatcagataagcggcaaactgctggacggcagttgggcgccgggtagccgtttgccctcagaagcggagctgactacctcttttaacgtcagccgggtcagcgttcgcagcgcggtgcagcgttttcgtgacctggggattgtggtgacgcgccagggcagcggcagctacgtgagcgaaaacttcaccccgcagatgttgagtaacgatcctcgtccgatcatgcaccttagccgcgaagagtttcacgatatgatgatttttcgtcagactgtggagttcaaatgcgtggagctcgccgtcacacacgccaccgatgatgacattcgccagctcgaagaagcactgaacaacatgctgatccataaaggcgattataaaaaatactcggaagcggactacgagttccatctggcgattgtcagggcatcgcacaacagcgtgttctacaacgtgatgagctcgattaaagacatctattactactatcttgaagagcttaaccgtgcgctgggtattacccttgaaagtgtggaagcccatatcaaggtctatatgtcgataaagaatcgcgatgccagcacagctgttgaagtgctcaatgaagcgatgtcaagcaatattattgcaatcgaaaaaatcaaatctacagaaacatcagggacaaaataacagttggttacaggctcaacttacagaactatttaacatagctgaatccgaaatagttgccattaacggagtaaagtgagccagtaacaatttactactatgttagtgtgacatagtcctactttaaaatcctggcagtgcggcctgaaatcagcggagcttcaaaaggccaagcccccccttaacgtacaataattttcgatatccaaactgccccctggaagatcaattttgtgctgcagcttttaattttctacgcaatgctgatatgacttaaaacgatgtgaaagatacagagtcagcttattgtggatggctggagattgacgaagaagatcttgagtaaagctcgataattcttctgacgttataactaacggatgtcgcgtaagcgagccttcagggccgcttacgagcgagtatgacagacgtataggggttcttcatgttctcagtttttgaattgtcaattctactagttcaactcaatactcttagcatgggaaaatttcatttctccaggcggctcccaaaccgtgattcctggtaagcccgcatcaagccgggcactcaaagaatgaatcggggattctgacgctggcctacaaccgccaggatattgacacgacgggccttatcattcacttcataaagtacatgctgcccaagtaagctaatacggcgaaccccttcgccatagcgttcatctgtttcatacagcctggggtaatctgccagacgatcggttgcagtgatcagacgctctgtcacgttccttgcatcccaaagagatgcattctcggccagatattcaaacaacgccgttatatcctcaatcgcacctggtcgccattctatttcataactcattgcgcagttcctgataacgtccctgtagttgctggcgcagttgcacggaactcattcccggccgcttatcggcgcgagcagcggcaactttcgctcggatctgttcgtcgtattccgcccgtgccagttcttcattagcgaggcgttgcaatagtgcagacttgtctgtactgccttctggaaaaatacgaggcaggctttcggccagctcgcgcaggaaattggcgttttcgtaatgtgtggtgctattcataattgactccccctttcattcaataaagggcaattgcaggttttttgatgccgcacgcatgggtataacgagtcagagtatcgatattcgccccggtgatattgtttcacaggcaggcgataaccggatgcataatgctcatttgcctggcaatctgcgtgttgtcaggttttatgcagagtatataacctttcagacacagccaccaacatatgcttatgccgcgccagcggcatgtaaggcggcacgctgtggttacatgtgataccggagtaaaaccgccgaagcccggcgtaagccggtactgattgatagatttcaccttacccatccccggccctgccagaccatacccgttttcagccatgagagagcttctgtgcgcggtcggagtggtcccgacgagggtttacccgaagtcggggcgtatctccgcgttagcgggccgtcagggccgcttacgagcgtgtactgagaacttccagccagaacactgacagcgatgacggagtatagttacaacattcataattaaaagcgactctgttccggccctttgggccggggcggggccgcttttcagttatgagggaggggctttgtggctccagttctgcgctggtccggggtttttctggaggttgtttttgtgtgttgtaactaaagtggctccggtcggggcccgccgcttgcggtgggaggtgcatatctgtctgtccacaggacaggcagtgaataggttttctttttaaatgaatgtaattaagtagtttaaaggagatataaacaggtgtttaaaagatacattgcaccctgtaaggctggcggctggcgctttatgacatgaacggttgtaaccttatggggaagtcccttgcagttaaatgtggataagcaaaattccccgtctgtgaggcgtgttttgtattaaaaacagggggaaccggatgctccagaaggtggatggtgaggttgttttttgtatgtagtgctgatttattgtgcactgacgggcttcaggc

SEQ ID NO: 3 - DNA, 56676 bp, synthetic construct
  source 1..56676: mol_type = other DNA
atagggaagtcgctgtaatgagtccatttcacggggccaccacctggttttttgatggttgcgtttatttggtacttaggcattcaaaaatacctccactcgatcacctgtctttcttgaacacatccagcggcgcagttctgtaaaactatcctggatctcacaacgggtacttttcattgtgacagttggtatattgtcgtagctggcatatgcaatctcttttttgggtgtgcgcaatatgcctttgtcaatatcaagaatcctgaacgcgctaattcttcttttgtgctcgttaaatagtgttcgtttagttgctgttatctgcttctgtgcttcctggtaaatgtaggtaagtacatcataagattgttgctgtgtaataaggaaattacggctaacatccgttgttgtgagcgttttattatttcttaatccccaataagccacgatgatatagagtggatatcctttcaaatctgccacacattcaggaatgacaggattaatttttaacttacgcgccatattttctcccggttgtttgtctgaaatagaaacatccccggaatacagcgtcataagctaattatgaaaagttgcagccactcactacaggctctttatctgattgttcatcatcatgcatagctaaacccccttacctggagtggagactggcaaatctggtgtacttctgcgttgcagcctgtacgacttgctcagggagagagtgagtgagattaactcaacgcgagttcaccaaataagggggtttaagtatgcagaatgatatttgcggataaagagtcgttacctgtgtagatggcgcggcacgtcactctgttacgtagcccgttgctgacagatgccagcggtgcttgaaatgcacgaataataacctccttatgccagggctttaggcaatgaccgctgtccggggttgttaaagagcatgagatgacaagcgttctaacgggcgtcaaagagtgctcattttccaggcaatgagcacaaaaaacgcgatgaacgagctttttacaatcatctggatagtattgtgtgtgcgacatttaaaacctgtcaatgccacgagagcgatctaatgatctggataacacctaaaaaatagaataatattctatttgttaaatgatgtacgatctaaaaatctagataatactgtttgtgtggtttattcgatcgtgtgaatgggtgtatgcagtcaaaaaaacttttttgaatatacaaaattctgaatttctgattttgtaaggttttgtatggggaacgtcagtaaagaaggggcttcagtagctgttctgaaaacgattttgaagcagtcacagatgtgtgctgaatcaaaaaggaaacgagccaggcttgaacatcgttgatcgattgcgcccatgctgctaaatttgcaggcaagttttccgcgatttgtaaatgcagaaaacgaggtgctgaaattttgttcgagaacgtaaatttcagataaagaaaacccccgtaatctcgtcttttccagggagcgcagattagcgggggttgacctcaaggcagagccttgagctgataacattttatttcaggtaatcgcttactacctgaagtggagtttaattccatgagaaggaaatatcaactcttacagtgcaagctaagtgcagcttgctgtgattagcaaacgtcactggtctgaactatcaccggaagagcaggtccgtttctggcaggactacgaagcgggtattgaaagctcattcctggtcccccaggaaaacaaaggcggaacaacaaaacgtcgccggggtgaacattccactaagccaaagtgtgagaatccagcctggttccgtcccgacaactataaggcgctgggcggacagttgggccacgcttacaatcgtctcgttaaaaaagacccggttactggtcagaacacactcagaatgcacatgtcgttgcatccattttatgtactgaaacgtcagagtgtcggtcgcaaatataaattccgaccggagaaacagcggctgcttgacgcaatatgggttgtacttgtgagtttttgtgatcgtggattgcacactgtcggcatgtctgtctcccgcctggcagaagaaataagcccaaaagacagtaagggagacgtgattcctgaaaccgcagtcacagtatcgcgcctttcccgtctgctggcagagcaggtttcctttggcacgttgggaacgtcagaaaagacgatatgggaccgtgaatcccgccagcgactgccgaaatacgtctggatcaccgaaactggctggaaaatgctgggagttgatctggtgaaacttcaggaacagcaacgtaaacgccttgcagaaagtgaaatccgtctgcaattgattaaagaaggcgttatccgcgagggcgaagaaatctctgttcactcggctcgtaaacgctggtatgcgcagcgttctctcgatgccatcaagtcccgacgtaagaaagcagcggagcgcaaacgtgctaatcgcctggcaaaactgccgtatgatgaacagcgaaacgaaattgcgcggtttattctgaagcgtatgccgccggacgaagcgtactggtgcactaaagaaagattagagcaactggtagccagagatctacgtcagcttgaactggccctgacagcttcgccaccccactagtttagctgcctgatacaatcggcactctcttcagccccttcggggcttttcgtgttgcctgcatttcatgaaattcctcatatttgacgaccgtttttcattttgtcccgctctgacagttttttgatggccgcaatagggtatcggagtaaaaacgccttcggttatccacaaaaacccgcaaaggaatagcttcagaaagcagcaaacatagaacatcaaacccgcaatatattcttaacccctgttctttaatcccctgcgttgcttcgccgcagggaaaatatttatctttgaaacaactgtggataattacaaaaatgccttcgcttgcagcggctaacgccgcgccgctcagaatataaaagtacctcccaccgcttcgcggcgggcatatggacttctgcctaatgaaaaacaaagcccctccaaccctcttacaaaaaaacgctgcaaatactcagccacagcgcccaacccatcactgaaaagcgccgccgcccccgcccgaagggcgggaacaacatcgctttcaataatggatgttgtaactaagaaattacatggctgtcagtcttctggctggaagtaccaagtacacgctcgtaagcggtcctggcggcccgctaacgcggagatacgccccgactgcgggtaaacccttgtcgggaccactccgaccgcgcacaaaagctatttcatggctgaaagcgggtatggcttagcaggatggggataggtaaggtgaaacctatcaatcagtaccggcttacgccgggcttcggcggttttactccagtatcatatgtaacaacggagtgccgccttccatgccgctggcgcggcatcaaaaaagaaagccccccaatgacgggggaaatagcgttttgatcaaaataataatgctagtgtgcttcttgcgtgccattccaggcacaaactaaatttaacgtttcgttaactacaaagaaatccgatgagttaatcacttgcgaaataaaaatcatatagtaaattactatatgattttgcgttgtttcggagaattaaaaaatgcctttgattgaatacatcaacaagttctatcgggggaatcaggcatcgttcgccaggctgaccggagttcaacctgcgcaggtaacacagtggatcaacaaaggattcatcgtggtgaatcacacactgtatagtccacgccgaaagctgggaatttaattcctggtcaatatttgacatcctccacgccctgaaggacggggatataaggcgcactgtatcctgttgtctggcagaaaaaagaaagccccgtagttaatttttcattaacccacgaggcgatcccaatacttcaacaacaccaggatagacttttaccgccctttacgcaataccatgtattttcaagtttctggtcgatatataatgttgttggcgcttgaggctttctgcctcatggcgaaaaggtggtttgtatcttgttgtgcggcagaaagaagaaagccccgtagttaatttttcattaacccacgaggcgatcccaatgcttgaacaacatcaggatagcctcttaccgcgcgttacgcaaggagaagaaggccatgaaactaccgggaaacgcccttatctggtgcgtattaatcgtgtgctgcacgttgttaatattcacactcctgacccggaatcgcctgtgcgaaatccggctgaaggacggatacagggaggttacggcaagcatggcttacgaatccggcggtaagtagcaacccggaggcgggcgaaagcccgcctttttggggatgatgtggttctggcattaagcgcctttacaaagggggtattctgtacggataccccctttttccatgtattttcaggatattaactggtgaatatggtataattagtcaatatgacagcaattattgaggcgagtatgcagcccaaaaaaacacccgtgattgtggtcaaaaaacgccgcgttctggtcatgccagaaaatcctgtggtgaatgaaaagccgcaggaagtacaaaaatcagcagttaatgaaaacaagaaagtacagaagaaagatgctgtagcagagaaaacacgtaaaaagcagcctcagccctggtatttgaaaaaacaaatcacttttcccccaaaatatccgaaagaatattttgaaaagtgtttcaataaagttcgtgctgtttttcctgaactatggacagacgaaaaaaagaacttgcccctgaaaagcgggattctccaggacgttgagaaatacctggcggataacccggatgtggatctgacgattgaagagtggaattgtgcggttcaggtgatgacgttccggtggcaatatcttcagaattgtactgtaccaggcgcaacacgttacgacctttacgggaaacctgctggtacagtaaaaaaagcacacgcaacttatgcgcaactggttctcgatgcccgtaagaaagccagcgagaagaaacaattaaaacgtaagggataaagccccgaagggctttatcatttcagcctggctgactgtagaatccagtgattttttccgttttcgtccaggccgtgttcgtaagctgcaagagccgctttcattattgctgtttttgtctggcctgttcgcagtgcttcccgttccagaatatcttcgtaaacgtccccaagccggacattggtcactttagttgctcccgattttttgcgggtagcttcattgatgaatcgggctgtgtctgcattggtttgttctgttttttgtcctggtattaccggagttttcaagtcaagagccattatatcacctttcgtgtgttacattagctttactgtgtaaagttaatgatgtaaagctgtcgtattacatccattagggattgatgcctaactcttgcgccaaaagctctatttgtgctttagctgtagaaaggctggatgcgcgacgtacatcatgaacgccagcgccttcattacaggccgcttcaaaaatatctaaatctgaaatacgtgtacgaagtggctgtatccacaccgggttctcccggagtaacttgtcgagatcaatcgctgcttttctgtggcgtggttttgtcgtgttaattctcgtgaaaagtacccagggttgcaggtctggattgatctgttgagctgtgcgaactttttctgttacatgagtcagcgtttctgcttcgaaatctgaagacggcttaacgagagtaagcagaatgtcagctacagttaaggcactgcgaaattccttgctgtcatgccctggacagtcaatgatcagcacttcacatatttttttcaggcgcttaatgacttctgatacatcgccatactcttcataaacaggaaccggtaataagccattctgctttcttttttcgttccagctaagaatatcattgtttttatcggtcttcagaatgatgaccgttcgtgaattatttaccagataagaagccaggtttgttgctgtggtgctttttcctacaccgcctttatctgatgcgacaagtaatgctctccccatagaaacctcggatatacagtgtaaaggtttatttgtctttacattgtattcattatagttacgttgtaaagatgtttgtgtctttacaatgtaattgttttttgcgctttacattgtgtgtatgttacagggtaacttttcaggatgcgattttgcctggtgtgttgttgcgcccgtagctcgctgacgctcacacgcgcagtacagcgccccgccctgctgcaatgcaccaggacggcgtaaactcgctggcgctcgcgctaactgcggcattctgcggcgcaaatcagcatgatcatggcctgtcagtccgttgggctttgcagggtgaagggatggccctgtgcatcctggtatgttcgtcagcggctgcgccgcgcttcggggcgatgtggccctgctgctccctggtattttctttcgccccttccatgccctgtcgggcatcacatgaatgtaactatatgtatttgatggtgttttttttgaagaatggagggaaaaatacttgataataggtcagttctgacctattataaacacatcgggaggggatggtccccaaccaccaaagccgctcagcggcaggagataaaaatgaaaacattaacttttaataacggcactgtttctgttggcgatgtgtttgtatcttcctggggatatgagcaaacgaacgttaatttctatcaggttatttctgttcatggcaaaaaaaccgtaactgttcaggagattcgcgcttcggttcatcggacccattcaatgagcggatataagaccccattactcaatgatttctgtggtgagccattaaaacggcgagtgcgtgattattacagtacgccagcaattgaaattgagtcattcgaaacagcatataaagcatcaccggaagaaaaacacgagttcacatcatactactaagaaaatagttgcagggggtatcccctgcaaccatgttaaattgttaatagtgaatacaggggcttaatcgcccctttttttatctggtgcgcagtaaaactccgtccttcaaggcgtggagaatgccaggcgaatgcagcaatgtaatgttgcgcccgtagctcgctgacgctcacacgcgcagtacagcgccccgccctgctgcaatgcaccaggacggcgtaaactcgctggcgctcgcgctaactgcggcattctgcggcgcaaatcagcattatcatggcctgttggtctgtcctggctttgcagggcgaagggatggccctgtgcatcctggtatgttcgtcagcggctgcgccgcgcttcggggcgatgtggccccgctgctccctggtattttctttcgccccttctatgccctgccgggcattgcatgaatgtaaatatttgtatttaacgatatttattttaaagaaaggaggaaaaaatacttgataataggtcagttctgacctattataaacacatcgagaggggatggtccccaatcaccaaagccgctcagcggcaggagcttaaaaatggaaaaccacgccaaatttgttgctactgaaattcttaatcagcttggtggaaaccgctttattgctatgacaggcgcaaagaactttgcctgttttgatgaaaatggtgagtctggtctttgctttcgtttaccttcaaattttgcaatgaagggaattaatttggttaaaatcaaattaaccttctctgatacatacctggtgacattttcccgtgtccgtggagctacagtaaaagaaatatcaaaatttgataatatttattgtgatcagcttgaatgtttatttaatgaacaaaccggacttgcaaccagattgtaatttttaatatggggggattccccccatatcaggaggataaatgaaagttcttattggtaatataaatattgataattatcatatgctgtctgcattagctgggattgcgggatttgacaggagcattgagtttacatgtgaaatatcagcaagtatcgaaattatggaggatgatttcgtaaataaagctggtatattaaagatgttagatgaatttattgaaaatgatttttcaataaagctcgtctagaaaatacttgataataggtcatttgtgacctattattaatgcattgagaggggatggtccccaatcgccaaagccgctcagcggcaggagttaaacatgaaacaatctacttttcctgctatcgtctctacaactggtcatgtgttttctgttgtgcgcgtgacgctttgcacaatttgccttaaacatgaaaaaacaggcgaggcttatgtcgtcatttttacagactgccataatatccgtgattataaaaaaggcgttgttccggttcttggggagctttatcaggaagatgtcgatttgattaccggaaaaagttaaaagaaaggggctatatgcccctttactgttaaaaggggagactatctcaatatgaagataagaaaaggtgacagacaatattatctgaataaagaaggtgatacgtttcatctggtgaagcgagtaaaaactttttctaagtctgcgactttaggaaaaacgaaagcaacggtgaaaactgttgccgatcttgtttttcatgagaaggcattcgacactatcgactttgccagcgacggattaagagagaatgataaagaaattgtttctatgatgattcaggaaatgagtgagggaaaaaatgccaaatgaaaacacaccagaaaacattaaacgtctgcggcaaaaaattggcctgacacagaaagaatgtgccgaaatatttagcatgagtccgcgtacctggcgtcgcaaagaagaacctgtcggaactgcaagcggtacagcacttacaccagtagagtttaaatttcttctgcttttagctggagaacaccctgattatgtactttgtaaaaggaataaaaagaattctgattctggaagtaattaaggaatattaagatgaaaatagaattaacgtatgacgaaatcacggaataagctacaaaaaccataactgaatttatggctggacttaatagccccagttgcattgaacaatgagcaattgcgcccgtagctcgctgacgctcacacgcgcagtacagcgccccgccctgctgcaatgcaccaggacggcgtaaactcgctggcgctcgcgctaactgcggcattctgcggcgcaaatcagcatgatcatggcctgttggtctgttctggctttgcagggcgaagggatggccctgtgcatcctggtatgttcgtcagcggctgcgccgcgcttcggggcgatgtggccccgctgctccctggtattttctttcgccccttccatgccctgtcgggcattacatgaatgtaactgtctgtatttgatggcgtttttttttgaggaatggagggaaaaatacttgataataggtcagttctgacctattataaacacattgggaggggatggtccccaaccaccaaagccgctcagcggcaggagcatgaaaatgcatttcacaacttttctaaaaaaacatttcgatatagaaaaagtcgttggtacttctgattctggaaatgataccgaatctatttatgtttatgaaaaaggaaatgattgcgaaccgttgtttattctgcatgaatcctggctaaatgcagaaattaaaaagtgtggggtatggactattggcaatatatattccacattggaacacggaaaagaatattctgaacaggaactaataaaaatgattaaggagggtaaagtaatcagcaaatactaatttggggttggtaacaggctgtggggcagcctgttaccaaatgacagatgcatctttggagattctcaaatgtcagtatgtgaaatgatagagcttgcaaaccgcattgtaaatggggaacctgtaaaaacacccgcgatagaagcatggattattagcgaaatcatttggtgggcgcataaccgggataatgctttagtttgagtcatatatggggagagtgtcatgctctccctttcttattgggtagcgcgttgtattacatagttagctttacgttgtaaagattttattgtctttacagtgtggtgcgttttccggcggttgcgctgcgcttaggggtatgtggcctcgctgctccctggtattttctttcacccttctatctgataacttttcttgaaagaggctggggttttatgaaacatttaaaatataaaggatatttaggtacagttgagccggattttgaaaataatatcctgcatggaaagctggcatttattcgagatctggtgacttttgaggcagaaacattagctgacctggaacgggaatttaaaacatcggttgatttgtatttacagtcctgtgtggaggacggaaaggagcctgacgcgccctttaaaggtgtgtttaacgtcaggcttgatccagagctaggcactggaaaaagaagtcattaatcatcgcgcaggggcttaatcgcccctttttgttgctcgctggcgttggtgttttctggtattacgatgtagtgtgttgtatttacaagtaatgctttacgttgtattacgatgtatactctttaattgcttgcaatctcttttcacgttcgttgctttgttcagcctttttcagttcactgaatatgtactcttcatctttacccgtgagttttgaaccggagagcaagcgattaacaactttacgtcgaattgtggggtaatctttggtttgagcgagtatggacaatatgcggtcactgctaagaatctggcctttccctgattctacataatgtagaaaaaatgcgattgctgacacgcccaaaagaattgtaaggacggctacaccatcaagacgaaacgttgatataaaggaatgcgcgaccactccaataattgccaagcagagacataaataaatgaagacgtttttgttgcgttgttttttttgtgcgcgatcatgatcatctatgattgccagtatcttttctggggttggttgagccattttttttccttatcagggacacggagtgtttaattacatagctttacattgtaaagctatgtttgtctttacgatgtaaatgatgagatgtgcagttaaaagtgtcaatcaggtatcatatagtaaaacactatatgattttctatgtatcacatgcatagcgataaaactggataaatgatttacactgtaaagattgaactgtctttacagtgtgtttcataagtcatacgaatgaaataatttgcttttgtgtgtatgcgtgtgtataattgttttcaggttagggaggacatatgaaatctgctgacctgttgaaagaactgatagctgctggttgcgagttgaaacggcataaggcaagcagtcaccagatatggtggtcgccaataacaggaaaaacttttcctgttcctcatcctaaaaaggacttgccgctaggtactgtcagatcgatcaaaaaaatggcggggatttaatccccgccgactttggaggtagccatgttcttttctgttggtgtcgaaacaccgaaagatgatcatacagcatacggtatcaccgttccggtttttgattgctttgattttggatgtgtttctgctgctgattctcaggcagaaatacccgcaatggcgcgtgaagcgatattggcaatcgtggaagagatggtaataagcggtgctcattctgttgatgatattcatgatgaagggtgtttgacttattccgccaacccaaattataaccactgtgatagctggtttgtaattgatgttgatttgtcagaaattgagggcaaacaacaacgtattaatatttcattgcctgatgtattgattcgtcgtattgatggatatgtgagagaaagtggtggcgtatacaaagatcgtagccattttctggctcaggcggcacgtcatgaacttgcatataaataaaactatccccgcgttgtgcggggattttttttgatagctaaccgctcgccgcagtccagcgaccgagcatagcgagcgagcgacgaggaagcggaagatccaccgttgccaggagaaaagcaggtacgcactatgcgttctgccctagtgatgctttcacaaaccacgcagcctgaactattacgccatgaatccagcgcaaatcggcatcacgattttcttttttcattttttcgccacttaaagccttgcttacgtgactttttaccagattttcgtgtaattcttttgcttcttcaattgtgaaaccaccaggcaaatttggcgcttctgcttccagcgatgccagtgcgatttcataagcccgacgctcaatattgtctcggacgtccaggctgcctatacgctctttgatttctttaattagttctttgtcctcgtcggtaaaagtggtcattagggactccttttcatatttatggttttcttgaatcaatttcgtctagtttgagttttatattttgctggtctgattttggtaattttctcatgctctcttcagttatttttttcatatcaagaaatgctcttgcatagaagcgcagacttgcctctttttcatgttgtgcaacataccaggttgcgccagaaaatgccgcagacaggcataatgtgcacagggcacaaattgcaatggctgttttgatactgaatggttttgctgatttagttagtatctctagttgttttttatggtcggaagcaatttggtcaaaattttgtttgattccagaaaaagtatttattgcttgtttggttatctcgctctgacactcattagatatttgtcttatatcgctttttgcaatattaatatctttattaatatcaatgctgctttgtattattttatttactttgtctgacagaacggcatccagacgagcaggtgtctcgtctatagcgttgataattaagtttgctgttttttcgatctgtttttctaaagcaatataatcttcaagaagatcgtttattttttgatctttaaactcttcagagatactacgagacattgtttttctccaattttatgaccgggtacatcccggtcatattgtatcagacgagattgaggtttttaaaggcaacatcaagatcgctgttgaatccattcatcaacatttttactgctcttaaatttgacaaatcttgccgctcttcctttgaagaagctgaacgaattaatgaacgaaaatcacgattgtcattatagacatcatcatactgtaggccgcttttatttaaaatattgaaaatgttgttatggtaaacaacaacaggattttcacctacaatttctttacatacgtcgtcagctaagaaaactgaatattgtttgttaataggggtgtccttttcagcaagattgaaaattactttaaaacgatcctgttcaacacccatagccattagtgttgtaatggtttgaattgagtctatttgttgttttgcttctcgcacaacaggaacgatgaaataatcaataaggtcatgacttccctgatattcattcatctgtacaaggaattgctcaatatttgatgagcctacgtcaatgatggtgcattcactatcatcaatacgtttgataatttcgtcaaactcctttgctgataatttctcgtcatttgtaccatcagtgtttaatgtttctacttttataatttctgacccctccagacgaggtttcaataaagtttggcaaatggtcgatttccctacgtttccgctattatttacaattgctagttttaagtacatttttcttaccttttatttcgtatataatttccaagagtattgctaacttgtacaaagttagcacaattccaagattttattgtttctatagatacattgttatctatagcttgttgagctatctttgcattgttgaagcaaactggcaaatatgcattaagtatatcattatcttgcttcgtttttctgtttttgtcctcaatggttattgctttttgatacgttttctctttatttgaaagaagatgagaaacgtcttttttctcggccctttcttttcgtatgcgatgtaatatgttttcaaacgtctttaacgttagatcaaaaccttcatcattaagagcattaacaatatcaatgtttcttatgccttgttgctgcgcttcttctatttcagcaaaacgctctcttattcttccagttttacttcgcttgaccttctgagctaagagcgaaccaagtagtttattttctccattgttcatttgtcagtctccaggacagtgaaatcatgatgagcaattctatcatgaacgtcaagcaatttttgctatgtatatgctatgcgtttgctatgtatatgctatgtcattggtttttatggtgtgtttttgctatgtattagctatgtttatggtgtgcatttgatgtgtgaatgctatggaatcaaataattttctaaaacacaggcacgataagctttgtacacaagcccgttccgggcatgttacaatgcttgtgccaaaggggacgcccctttgcaaccccgaacggcgcagccgttctagtgcggttctacgaaccttgaggacttatgaagaaaaaagccaacaaaagtgttcatgtgacattcagacttacagaagaagagtatgctccatttgacagagcaatcagagagcttgaaattagtaagtctgaatttttcaggctgcttactattggtaaaataaaaaactacacatcagataaacgccatatcccggaatacaaacggtgtctttctcagttaagttgggctgggaataatattaaccagatagctcaccgcttaaactcagatcatttaaagggtattatatcagaggcgctttataaaaagattttaaatgtgctgattggtattcgtgatcgccttcaggagatagccaaatgattgtcagatatggtggtggtaatgacggtattgttgattacctgataaatggtcgcaaagcagaacgtcagtacacacgtgatgaacttgatcatcgtgttgttcttgatggtgatctccagactacagataagattatcaattctattgaagacaacaaacaggaccgttatttgcacattacgttgtcttttcatgaaagtcacgtatcaaatgaaatgttaaaggcggttgtggatgattataaaaaattattgatgaatgcttatcatcctgaagaatattctttctatgctgaagcacatttgccaaaaatccggcatattcaggataatagtacaggagagttaattgaaagaaagcctcatattcatattgtcattccaaaggttaatctagttacaggagataaacttgatcctgttggccttatagggaaaggtcatactatcgaacagctagatgcaatacaggagcatattaacaataaatataatcttgccagcccgaaggactttccgagaaaaaatagcgattatagtgttattttaaagagagtaaaagcagatcttgatgtagagcgtaattctgcatttaagaatgaattgaattatcgaattgaaaaagaaaaagtaagtgattatgctgtattcaaacagattgttgcagaatatggtgaggttaggataagaaatgaaggaaaggccagtgaatatattgctgttaagttaccgggagataaaaagttcactaatctgaaaagcccgttgtttcgtaaaaactatattgaagagggaaagttgactcttcaaaaacctacgcctaagcaaatagaaaaacgtctcaatacctgggtaaacaaaaccagtcacgagattaaacatgtttttctcaacgccagcgttcgtaaggactatacaagtttatctgaagcagagcagattgaatttctgaaagagaggataaaagaatatgactcaagagaaaaacttaacgaaagaaattctcagcaaacgtcaggacgagcgggaggttacaagtcgtgtcctaaaaagtttgccagaatccgtcaatctgaagcaacagtcggattgtcacgtatgccccaacgcggtatggtttacggaatcaatggattcacaagacccgactctatcagtgtattgtcagatatttcgcagcgtgatctggcagagcaattaccgcaaagagaacatcctggtcaggatgtgcgacgggattatgatagacaattcacagagtcaggaataaaaagtcttgaacgctcttcttttttgtgtgaaacgatgtttcagactctgaatgaggcggcagagaaaaacgaaattacaacgatggcagaaattcgcagaaatattgatcctgtcaggtttctgtcttctgccgccgagcgttttaatattattccagcgcagcataaaatcagaacagcaaaggacggctccccccggttttctgtggggaatcgtaatatgaacgcatctgattttctgacaaagcatattaatcttgcctggaaagatgcgaaatcctttctgcttgaagtttattcacagcagttagaaaacacgccatatacgcgctatccaacctacagacgccttacgcatcatgaagcccgtgagcgtcttaactcactgaatttatcggaaaaaacattacgaaatactatcaggtttgagcgtggcaagctgtataatgatctgcgagagatgagacgcgagttgaagttaataccacgtgaacagcgtgatattgccgtgggtgtgattgtttacaaaaaactaaccacacttgagcgtctttctgaactcgacacagaggggcgacacataattcgtcaatatcatgctgactggcataaggataaagatgaaatgaaagcccttgaacgtctcaaaagctatctcaacttcgatgaaatcaacgccatttctgctgacgaacccgaactttcgcttcagaaagcggtggattcccagcgccgcttagaagaggcgaaaaaggttaactccaaactaaaagatcttgtgatggataagcaggattccaggattgtttatcgcgatcaggaatcggaaaagcctgtctttaccgacaaagggaactttgtcgttgcgggtaaaaatccctcaaaagaagagatcggaataatgcttgagtattccagggaaaagtttggtggtgtactcaaacttaccggctctgaagatttcaaaaaaatgtgcgctgaagttgccgcagagcaggacatgaagattattttacgcccggagcagtatcaacagatgatgctggaattaaaagcagaacttcagggtaataagtttgagcaggtggaaacacaggaaaatagccaggagtccgaatccagaatagagaaaggtgatgcattgaaggaacaggccaccgaacaggaacaggctaccgaacaggcacaggccacagagcaggcacaggccacagagcaggcacaggccacagagcaggcacaggccacagagcaggcacaggccacagagcaggcacaggtcgcagagcaggcacaggctacagagcaggcacaggctacagagcaggcacaggctacagagcaggcacaggccacatcttcatatgatcctggtgttattaccagggcaaacaccctggattcacagatgcttagtaaaggcacaaatggtgaatttggctatttgaaatctctggatagtgatgaaaatgagatttgggaagtgctggggcatgtacctggagacagcgatgatatttttgacgttgccagttttgataatgaaaacgacgcaaaagaattctgcaaaattgttaatgagttaggtattgacagaacacaggctttgattcatgagcagctttcttctcaacaggagcaggctgctgtgcaggagacgtaccaggaatcaccagaagagatactcgccagtatttcagctaacgagcatatgatctccgggcttgaaaacttcctggttaaagaccgttctcagttcagttcctgcaatggggatatcgttgtggaggcggaaattaccaggggggaaggtggtctttatcaccttgcggtcgctggtaagcatggcttagagcgtggtgatgcagttgccagagtggatgttacggagcagcaattcgccgcgatcactggaaaaacaccttctgaagttttaacgggggaccaaacatcggctcgtgttccggtaattacaggtatccatttcagtacaagggctattgaaaatgtgaataaactggaacaacagaaggattatgtctatttctcaacacatgaaggcctgaatgctgaaataaaagatttcagctctctcaaggatgctattgagtggggccgggttgaatgtgagctacatgatctcaataagcgcgatactgtcatttatcgcgtcgagtctgaacatatttctcaggggattgacgctgtaatgaaaaatgctgaacgcgtggagcgtcatgaaattgagaaagcacagggacgggattgcacaccggaagacggcaagattcttgaggcaattgatcgctttgaggacaagttcagaggggaaggactgaagttcgaaagggagaaggccgaatccgatttgcttaatcacggattcactcgtgaaatggcagaagatgctcttggaaaacaatttgtccaggccagagaagatcatcaggaatcgcagcagcagcgtgacgattcacaggacatgcactaattaagaaaaaagcccggttttcgggctttttaagaaaaaaagtcaaccccaaacagaaatagcagctacaaaaatggacagtaataatgcaataaaagtaatgttgagccatatgttggacgagtcttttaagactaatatggctgttaatgccattgtaatgaaactaatggtaataatggctgcaaataaatattgtacaatgttttttattgtacctggttgatccccaattctttctctgtaatttgcatcaatattagatatccattgaagtaatagagcaataaaacagcctactccgcaaaaggtgaattgcacagacgtcgggtttgaatcaaacaacgagatcgctaagttatataagtataccgcgcctgaaatcaccccgacaactaaaatagagatgagtataatccgaagaactaatgtattgctttcctttaaggaatatattaaagccgctttttcgtattctttcatgatttaatccagtctataagtataagaaccaacaaaactgtccaggattcattttctgtgagcatatgaaatcccatccagagtgatgatactggcagaaacacgcccacagccggaattattagtctggcggagaatatccttatcaggagtgtcaatagtgataagaacagaaaaagagtgatcatgtggaaaaacattcacatcacctataacactattaagatagcctgccagatatccatttcagacaggctatatacagggttcaattcattctatgaaaggtgtatagggattgctaatgtttgtgtcgatcaaaaatctaatattaatagatgagaactaatatacatatttccagacggatggggggattttatcgtagagtttattcattgtgagttctgcaagcctgtgatcggcggcggcattaaacgactcaagctcagaaggagggaggctgttttggttttttcaataatcttttcaagtgtgtcgagtgttgagcaacgtcttaactgaaagagccagtcttgttttgttttcattgtacaattcctgaagaacatccttgtcctatagtagctacaacgatgtttgatttctactgctggatttatttcttacttttttgtgtaaactcaaatcccagtttcccggttgctttgtcaaccagttttacttttgctgaaaattctttgtttgttttttgactaatgaaacctttcaattcacttgtaactccttttgttattaacgtttcaatttgtttatcggttaatgatttgccgcagaaatctttccatatcttaaattcacatcctgtacaggaatagctttttggcctgataacaatctgtttaccacaggaaggacaaggagaggacaggcgttgtgactgaccagacggcgcgctgggggaaacttttatttcagcactgttcgcattactgatcatcggtatcagatcattgtacagttcgtcaacgaactgttcgatggtcatttctccgttttcaatgagtgtctgtttttccgcccataatgccgtcatatctggattaacagctatatccggcagagcatcaataagcgcatacccggtatctgtcggaataagttttcctttttccagggtgatatagttccgttttttcagtgtttccaatatggaagcccgcgtagctggcgtaccaataccgccatgttcgtcttttttatcccgatctttatccttcagtaactttttaatcactggatcagtgacaaaatctgcgacacgaacaagcgcggcaagcaaagtggcttcggtgaataatggcggtggcgttgttttcttctcattcacaaccacttcttttgtcgtcactgtttctcctgtgcgaattttacagagaagatcaaaagcggaatcatcattttcgccttctgcttcatcgtccccggcattttccacaccaagaaatgcttcaaatccgctgtctgtagttttcctggcacgggcataaaacgactcatcaccacactgaatggcgacggatacttcctgatatgccttttcaggcatgaactggacaagatagtgttgtgcaattgccaggtaaacgttgcgttcgtcggtgctcagtgcgtttacgtccggtacgctaacggttgggatgatggctgtatgcgctgttacttttgcgctgttaaatgccttgcttttacgtgtcgtatcaatatccattggctgatcaaacaccgacttcagggcatcgattatttgtggtgcttcactgaattgttcgtctgaaagatatgaacaatctgaacggttgtaagtaattgctttgtatttttcgcgtagctgctgtgtgatatccagcgttttttgagcagtcattttgaattttttattcatgtactgctgaagtcgtaccaggttaaagggcaacggtgcagccgtttttttatcatctgttgctgccgcttcaactgtggctggttttcctgcaagtgatgttgctgttccattcgcccaggttttatcaagcaatttgcggtctgtcagcggtgcgaactcaccaggtttccagttcgccctgataacatcagcgccgcgttgaaaatgtccggtcatggtgtagtagaagctggatttatgatttttattggcacgggtgcgatttacaatcaggccaagcacgggtgtctggacccgtccgacagacaaaacacctttatagcctttagtttttgccgggattgtgtaagcacgcgtcatggataaaccgtatatcgcgtcggctacagaacgcgccagagccttgagatagagtcctctgaagtcacggttatttttgggatttgccagtgcctttttcactgccggaagggtgttgtcgttgatcagaacgcgcttaacgggttttgtgttgcctgcatattccaggacctcatcgacaagtagctgtccttcgtcatctggatcgccagcatgaataatttcggtcacatcggcacgcctgataagctcaataatcgttttaacttgttttgcggcgctttctacaggctggtattttacgggataaagacgcaaagggagtgtttcaatcttccactctttgtattcaggattatagttttcggggggctgtgattcgataatatgaccaaaacagttagtcacaatagtattatcactttcaaaccagccatcatgacgggtaaaattgccaccaagcgccttaacaatatcattcgctactgcgggtttttctgcgataaaaagtctcattttattatcctttaaataactaatgtactttcaatagatggttgtcttttgaataaagagaagtcggtgtttaatcgttcaggctgaatagtgtgaagtaaaaggctttcttctctggatagaaggaaatcaccccattcgaactcttcccacacatcatgactgttttcaccaggcttatttaataaggactccattttgggctggattatggctggaggcttattttcataagcggaatgcaccctgtcactccatttacaaaaagcaccacttaaaagaatttcctgtgacaccatatcaaactcattatcttctatataagaattaacctgatgataaattaaccccatatcttcaatattccaggtgtatttggtagtgaaccatcttagctttttatctgtatgcaaaagaagtccattaactgcctttctgcctttccgcattccaacccatacacatttattaggccacggcctccctacagacaatgattcgccatcaattattaaatcaagttcaggtatggagatggatttaagagtgcatttattcgccatgcgatgaaaaagtttagggacaatatgtaggtacatatctatttcctttatctttatcgtagtcaaagaagttctgatttgttgacctcatggtaaacaagttcaccattgcgtattaaagataagtctttaatattgcgcaaaaataaaaagacagactcattagtatcattaataaaaaaatgataaaaaactcaaccatttcaggtatattaaattttctgtgtacagttcttaataaaaattgacccgttgccacccagaaggcaaagaaacaaaccacgaacactgaattatcgagtaaatcgtataacgaatacatccagtgaccagcaggatatggcaaaccaaaagaagaatgcatttctttcccccattatgattttgaagtattcagcattagtattcatcttcataaggtagccaatcgtaatcgtttgagttccagtcaattggaagaaatggctctgtttgtttatggcaggagaggcaaacataaatatttttctctttatgaaaaatcatcgacttacatttatcacagtcgcaccaaccctcgactggttcagggcattcaggtaattctttctcctgatgtttaccatatttttcccattcctcggcatcctgataacctccccatactgcttgtgtaaaactatcgatatagtcacctgactcatttttatcaaaattaaatttatatacttcgccatttagttcaaatgacatatcaaggtcaggtagtaattgcttttcgaagcgttgttgtaactctttcttttctttgggtgataaagccataaaaacctctatcagttagttatgtttgtatcgccactcccacggtggaaccggatttgtatcggcccacaaaccgcgtttttgttcctgcgcttcccgttgcagagccggtaatgactcatccacgttgtaacgtccatatacccatgcagcgccagactgaaccataaagcggctggcatccgtgccgttcgtcgtgaacacatgaccgataatgcgtccgtagcggtctttatccagtgtgccgtaaaaccccgtccttcagggcgaggatataaggcgaggttttccacctggctttttttgtttaaaatggtcgaatgaaacgactacaggcatttaaattccagttaagacccggtggtcaacaggagcgagaaatgaggcgcttcgcaggcgcatgtcgtttcgttttcaatcgtgctctggcgcttcagaatgagaaccatgaggcagggaacaaatatattccttacccaaggatggcctcgtggctggttgagtggaaaaatgccactgaaacccaatggcttaaagatgccccatcacagccattgcagcagtcactgaaagaccttgagcgggcctacaaaaatttcttccagaagcgggcggcttttccccgattcaaaaagcggggacagaatgatgcattccgctacccgcagggcgttaagctcgatcaggaaaacagccgtatttttctgccgaaacttggctggatgcgctaccggaatagccgtcaggtcacggggttgtgaaaaatgtcactgtcagtcagtcctgcggtaagtggtacatcagtattcagacagaaagtgaagtatcaactccggttcacccttcagcatcaatggtcgggctggatgctggcgtggctaaactcgccacgctgtcagatggcactatttttgatcctgtaaacagttttcagaaaaaccagaagacgctggcgagacttcagcgacagttaagccgcaaggtcaaattcagcaacaactggcagaagcagaaacgcaaaatacagcgactgcattcctgtatcgcaaatatccgcagggactaccttcacaaagtcacaacgaccgtcagcaaaaaccacgcaatgatagtcattgaggatttgaaggtcagcaacatgtcaaagtcagcagcgggtacggtcagccagccggggcgcaatgtccgggcaaaatcaggtttaaaccgttcgatactggatcagggctggtatgaaatgcgccgccagcttgcgtacaagcagctctggcgtggcggtcaggtgcttgctgttccgccagcgtacacaagccagcgttgcacgtgctgtggtcatacagcgaaagaaaatcgcctgttacaaagtcaattcagatgccaggtatgtggatatacagcgaacgccgatgtaaatggcgctcgtaacattttagcggcggggcacgccgttcttgcctgtggagagatggtgcagtcaggccgctcgttgaagcaggaacccaccgaaatgattcaggcgacagcctgaacgtcgtaggaatccacgtcctttagggcgtggaggatgtcaacaatcggactcggatcggaacttcatacacaacaatttttgcgggtagtgttttaatttctatggtgtcgccatcgagaacgcgaataactttcccctgaagaattttctggttaaatgtataaggtattccacctgcatttgcagactgaaaagcggtaagcagataaataaatattgttaataagattttcataaaagattaacgttaatttgtctgtgcaatggatagagcgttattcttattttcttcctgccgcttcatcgaaaaatgatgattttccttctgctgcggcatcatactgaaatgaatatccaaaatcatcgtcatgctcaaaacctgtattttctccattttttagtctggaaagaataatctcaagttgactgattatatcttccctttcactatgacgagtgccttgtaagagaatagttacttgttgcaaaggatatgcatgttcaatccatgcctggctaggtccagagtgcgttgtatataaatttttttgtgccgaaatattattcagaaaacagtttaagtcatcattggaataaatttctttaatattgttgatgttggatgttagttttacaagttggaatggtggagaatttttgtcttcattgttgtctcctttatcgtattctggaaacacaattaacaaaggtgcaccattagttctcttcataaaacgggttactagccgcccaatatatttattggacatatgctgttcaatgtccaggacagcatattcctccgctgggaacattttgtttttttcgatggatttaatttttctggtaagatgaatgtttctgataagcaaacctactatcaggaagtaaataaatatccagaataaaaaatcactctgtagttgttctatcataagtgaatcctctaatttgttaatggacaaatcattacccgttcatcaatgtttgtaggaagttcgccaggagtagggacgcgttctcctcgctgggcggcttgtgccagtgttcgtagcagcaacacgctggcatgaaatatggcctgttcttttgtggctgcgcttgtttccaggtcgaaatcactcagagtcacaacgacttgcccgtttctgttctcgaccgttgccgggtagggaacaaaatagggagcgattttgctggtttgcttgcgtcgggcatcgagtttactgacaagttgataagcccgaaggtgtgtgtatcgtttcaacatgttcagggagcgatggcctgaaatagcggcaacctcaatgacgttcaatgtaccaagttcgaaaaatcggctgatagcctcatgacggaggtcatgaaaatgcaagttttctatcttcagttcctgaagtgctgttcgccaggcacttttgaatcccgaagaggtataagaaaaaatattaccgttaagctgtgtcggcagcatttgcaggtaattacgggctttcctggatagcggaacatcgcgtggagcaccgtttttggttgttggaagatgagctaccccatgttgcagatcgacgtgttcccaacgcaatgacaagatttcaccttgtcgcattgctgtttccagcgccagatgaaaaatgacgtaaagagcctgatttttttctttgaaataccgcgaaagtcggcgttcttctcctgaagtcaggcgacgatcgcgcccgctactgattttgggttttctgaccagttcgacaggattcatgcggcatgttccccattccacacgggcgatattgaataatgatgacaacagggcaagttcaagacggacggtgtttccggttatttgacgccccgttcgtgggttaatttgcgccaaccgttgatcccgataattggcaatatcaacagtggtaatgtcatccatatagcgttcagcaatagggtgacgtttaatcacatttacgcgataaaactcctgtagatggccttttttgtggatggatactgtcttcaggtatttgtccagcgcccggctgattgtcattttgcgaattttgattttctgaaacatatcccctccagacaaaaaaggggggaaggtagagattcttgcttcgaaatgcaatatcaccgtgccaatccggtgtgtggagaacttccggttcctctaacggtagttacagtaatcttggatcacaccgaggatcatttactggtcgcaatacgggaagtggtacgctgtttgtttacgcctctggagggaatggtggttccgccgggggagattgtgctaacacatccagattgcagggatacgttgcaggggcattgattagcacaaatgcgagtaataacccatcttatgggaagacagcattcatctcttttgctgtccctgctggcgcaacttatcagatcacctcttatccggcccaaaactactcttgtggctcaggggtattttcagtatttggataccaaacttaattgagagttacacaggacgcaatacaacctgtcttgtgctcgtatttgtaccattttcgtgaaccagatggactctcgactacctggcagtaatgagaatcctcagcatttcccattctggcaatggcacaaaagtcaaattgccccaagtaaccagtgctggaaagttgagtaaccttgagctttccacctaatgccctccacacaccggattggcacggaggccagtgccaatccggtacgtggcagaaaaatggcggcggtacagtacagatggttactgcgacagcaagtgactggaatcagccaattgccaccgcgacttgcccttcaggaaagaaagttacaggaggtggtggaatgtgcagcttttcaaatgccattctcaagcgttcttccccctccggtaacagtgcatgggtagctggttgcagccaaaacaccaaccagaatcagtattattcagtcactacttatgcattgtgtcagtgagtattctggtatgaattagcggaagcagtgaacagtgatgtagtagctgccatataacgtaccgttatacgcactccagttcatctcgccgtttgcatctggattgtctgtaggccagacacgcgtcatcgcggtttcctttccatctatgcgatatgtattaacgcaaagtttgaattttcctagagccttcgttgtggttgggtacaaggtgaaggatgttactgatgtccacggcgcagatgaacctattgatttccacgaaccggattggcacccctatttctatgattaatcaatacatactgcatcacaacgataactagctattgaaggtgggtaagctgttagttgccagtttgatccggacttagataacgagcattctgaataatcgtttgaattatggaaacccgttgctcttacatgggataatgcgcaaaaagtatgctgccccaagtttgactgagagtatgggaaattaagctgataggaggagactttccctatgcctcctccttttttccacacaccggattggcactggcctccgtgccaatccggtacgtggagaagggcatcaggtagcactgttctgacaggaaaaatagcaaatggacaacagatcccattgccatctggtttttctgccagccagtgtacctggagtgtcagtaatgcagaaaatccgcacgggtggaagccgaattattttgcggggtcagttgcaacctatgatgccaatcgaattgtgaaatgcggcttctatgacgaatacaacttttatggcggtactcaccgaactgacctgtcggggaaatgtagttacatcgtggtttgccagtgataagtatcgtcactggcaaatggcgtaaactgtaattgaaccattttgatcttcggtcgtgtcgcaactggcagtccatgcattgttgcctgaaggcatggaacgtgtgagccatataaatccggtatttgatctgcattgaccgcctccaccaatgactttcttgccagatggacatgttgccgttgctccaggccaacgccacgcagtagctgaagcggtaacaatttggctatcacctgcgccaatttttttccacgtaccggattggcacgaaagtatcgcccctgtactgtcgcggcctacaagcccgttaggggagcaggaagcgccagccgttgctgttttttcaagctgtaaatactcgcctgtatagaggcgtccatcagcgcgtacagttccaccttttacctggccgccagtgtaaatccctttattattcacgctgcgcaaccaggatgaatcggacatataccagccgccaccataggtgctgttcatccagcctttgctgttttttgtgaccagccagccattattcgaagtgatatcaccgttggttgtggctgtatttcctgaaattgcgccagagaacgtaccgttacgtgcgctgatatcgccggagagcgcggcggcgttgccgtttaaagtgccgacgttgttcagattgttgctgttcatatcaatcgccgtatgcatcctgttaaggtcgggacgattagttatggagtaacgatacaaacggtcgctttcctgcaaatcagcgccaacaagtgccattgcaatatgcccggtcgcgcagttcagcccatagtttgaaggcgtgtcagaccagccgccacccgcgccaatcgcctgcttgctgctgttgatgtagccgccaagcccttcaatcatgcttgctaccgagcgcattccggcttctgacagggactgaccgccattactacaggtcagggcttccagacgggatgttttgctgttctttgcaataccggtaatgtagctttgtccaaaattggattcagaaaaaccagatttcagatagcctttctgaatgagtgttgctggcgtgattgttttgggtaaagagccagcaagtagtgttgatgcattgtcagcaacataattacgaacagcggtgttataggtatttgcgtgttctgctgcattctgccagcgaacattattcagatggttttcaatgttttcataaactttagggataaccatcaccattatgccgatgacaagtaatacctcaaggagagatacacctttatctgtctttttcattgctcttaatcctcagaataatattcccggtacgagtgagtaaacaaacattccgccacaaaggaacggaccgaatgggatatattttttttgcttgtttaatccgccaagagtgaagaaaataattccgccgataacggcaataaatacaattgaacaggcaatctcaggcgttggaaaccatacgccagttcctgcaataagtagcacatctcccatgccgaaactttccgttccgtttttatatgcaaagtaaagtcttacaaacagcataatgaagaacatcagcattgcacaaaacaaaccagacaatatattgttattccatgtctgatataacaggccggagaagataaatgtcacggtataaaggcgtggtagtagaaatgaaagaaggtctatacaggccattaatgtaaggaaagcatagaaaacaatcactattatggattgaatcagtgatagctggtttaatgaaagatacagaacggcaagcgttgtcagtatcagacagtaatatttctctgtgccggttttataagatacattaagtttatttaaacggaattttacttcattgtaggcgtagcattgacagacgatatttgcacagcaaagtatcgccgtattaaatatgctgtttagcataagttaccttaccatgtatttattgtagacatagcgtgcgtatttgattcttttctgttcgttactttctttgaaaccagcgttataactgccaagacaactccagtttatgccacatttctgaaaatgtgttgccagaatccacgcgccaatttgtatattgagacaggggttatcaataagctctgatttgtcttttattacgccaagttttttcaggttggggatgtgtgatgaattaatttgcatcagtccgtaatcggtgcttaatatttttcctttttctttgttgtgatttatccggtctggtatgagtgaactttctacagtggcaatgcttttcagaagcaggggatcaatcagataacgttgcccggcttgtgcaaagcaggcatcaaatcgggtcgttgtcgctgaatatgcgggttgcaatgaaaagattaatgatgaaatacatgatagcaggatataaagaacagaacgcacaatactcctcctgtaaacatggaaaaaataagcgcccgaaggcgcttatccgatgcacatgaaaaatcaggaatcagtgctgaaggtcagcgtattgctgtcagatgagcaaacagttgccgcatcgacagttgatgtggaagtgttgttaatcttggagattgcgctggaagaacgcagggcgtttaccattgccatgcagtttttcttcggtacgctggcctcaacaacattgaaggagtatttatccgaagaggttgtgattgtaacggagccgccccacgggtttttcgcgcttgcgccagttgtatctgcgatcatatcggatggcagtaatccttgcgcgtagagagtcttaatatagttactgtcagtataacgcccctggaatttcagtgatttcatgttggcaatcactgtcaggacattgttttgttcattggatgattgaatgttggattgcaccattgaatagagtttatatgcacttgctgcaagtacaacaataacaccaacaaccaataacacttccatcagtgttgcccctttgtcctgttcttttttacggtgtgcagaaagagaagaaaaaacagaacgcatatttaaaatattaatagaagacataataaataattccttatttattgagaggttaaaaacattagggattagtgttgcaggacgttactgacatcgtagagagcagtaatcatcattacaataaatgcgaaaacaattaatgcggatactacagttatccagactcctgttgatttgacttttccttttgcttcctccagccatttatcagcatagttagacatcatcaatgcattgtcagcattttcagaatgaagtaatagtttgttaatgcataatttcgatgggaagtcataaccgcaacttttcattgccattgcaagtgactgtccaagtaaaacatatctcctgatttcttttattcgttcgagtagccatctgttttgagtgctgtttttctccagcctttccagtgcttttgcaacaggaatatcaatacttaacatggaggccatgttgaagagaaaacttacaccattaaatgttttataaagtgaccaggggggaatgtaatcaagatactgacggcccggtcctgtataacgtgcaagcgaatatctgatcaggaaaaacagtacgatgcagaagcagagaatatacgttccgttttctatcaggaaaccagacgtatctgtcagtaatcgcatagaacttgtccagcgttctttcggtgcgagagaaaggaaaacaggtgttatggtatcgtggaccatatacatgtttgccagacaaccaaaaaccagtacggatggataaatcatcgacataaaaagagtcattgtcatttcagaacgacttctgacaatttttatggcctgaactaatcccccggcaaggtcgccgcgaatttcaccgctggatattaattgatattcatcctcctttatatatttttttaatgctgaagatattgcccctgtgttattcagtgcattaaggcactcacaacaaatatagtaggttgcagaagaccccgttttctttccgtagtctgtttctacatcaatcaggtgtgtaagagcacgctttacatttatgccatcatttaacattccagataaatcttcatataaacttattctggaatacctgaatgtgtattttgccagccagtgggatattccagagaaatcaatttctatcgaattgagtttctcaaatatatcttttatcgccatgtcccctccgggagcattgagtatttatcttcgtcaatgggggatatgaagtgtgcggacagcggatctaccagaccatgattaatcaggtgtaataagtgctgattacgggtaattcctccaagttcatagtgccagtatgattttgcttccagacgcccggactgacgatataactgaaaaaaacgtgcgtcgggtgagacgacttctgctataaccgtccttcccgttacgcctttataacagtgctcgcaaccgtcaagattgcgcaaccggacctgttccggcaggcaattttcctcaatgagtttccgttcttctgtactcagtttgtctgccatttcagcataagtgcgcttacagtaaggacagagtttttgtaccagccgttgactcagcagaccgataaacagaagaggatctgcaatcagcctggcctgaatattctccatttcaaggcgctcaataatattcagggcatcgttggcgtgtaaggtcgtcaacagaaggtggcctgtcatcgaggctttgattgctgctaatgccgatgcgtgatcacgaatttcgccgttcagtatcgcgtctggatcaagtcgcatagcggatttaatcgcgtcaacccagccatcgacggaatccctgactgccgtctgtattgcgccgggaatgcggccctccggcggtgattcaatggtgaaaaggcgttttcgggggagacgcatattatcgttatggttgaaaccaaacgtgctcagataggcttctgaagccgttctcagagtcgttgacttaccggaccctgtagggccggataaaacgataattccttccgggcgttgtaacattcgcctgattgtctggatttgttgtggcatgtagccaagttcttcgaaagtgggaatatcctcgctgtcatctttaatcaggcggaagaccgcgtacacaccacccacgcacggataatgcgaatagcgcgccccaaaaagatttacaggcttcagaaaattctctgcgatacgtgcatcctgcggtattgccgggtcaaactgcgtacctttgataacatcgctcattccagaataggcagcacccagcaattccagcccttctttacgcgggatgacatccagaatttccagttccccatgcacgcgtgcttcgacataagtgaactcggaaccgtcccgaccaggtgtgatgtgcaaatcggaacttcccaggttgtttgctttcgccaggtagctaatgaccttttcctggttgctgtttaagtgttgtgatgagtttgaatcagaccttctttcgctgtcctggtagcgggatgcgataacatcaagagtggtccaggttatatttacctttccgggatatttaacattcagagtgccaatataagcctgaacagcacgctgtcctttactgttatcagcgatataaacatgaacaggtttatcgtctttttcagatatactgacgtatacaacatcagaaatatcttttccgggatagtctgcgtcagaaaaaaacattatgtgtcgtctctttcaatagcattaaagaataagctggcgtttctctgtaccattctgaatccagacgccattaccagtgattgatgccaccttataagaagtaccaggaatactttgcccgaccttgacatcagttactgacccgtccaggagagaaattcgtgctgacatttgttgtggtgtaccaaatatttcaataacacgaagtgatttagcggctacacgttgcgctggtatatcggtatctgcaagagaagaatacccggaagagggcgttattgctggcgttgcggacgctacaccattcaaatcaagattacgcaattcactgacagccttctgacgcgccagctttgcttcgtaaattaccgtttcagcctgaatctgctctacctgtaaaatggtcggagatgaatattttatttcgtcttttggtgtgctttcagattctgttgcgtaagcaagtgaactgaaaaaacagacagaaataatcgttggtttaattatttttaacatagaatgcacctttaatctcataattcagtcttccttcttccagttttattcctgcatactggaagcgaacggcaggttcatgaaagttagcgaaaagtattgaaggcggggtgctggtctgaatcattaattcatactcgttccacgggagaattattggacggccttcttcatctgtttttgtgttttcaatttcctgccaggtgagttttaacctcattttttgtgcaaatgttgtcaggcgttcctgaatatctgttgcctgtggcaatgtgtccggggtaattggatcaggcgaaacgtcaaaactgacaggaagagaaaatcccccggcgctgccttctggcaatacaaaagttgctgtggttgtcccctggaatatttctcgtatccgtgtgctgaagtcctcgaccgtaacgccgctcagttccttatatgatgtgcgtaacagcccgtcattaccggatgtgctgcattcagcaaggtcaaaacgccatccggcgatcgataagggcagtgcatcccatttatcaatgcatttatcaataaatggttttatttcaggtaaatgttgccacggtggggcaatctgtattgcctgtttgtctgccatttcttttgccagacgaagacgtgcagcttctgccgcagcttctttttcccggtattcctggtacatggtgatcccgttccagagcagtatggccagaatggcacttccgccataaatcatgaactgacgttttcgggatacgcgagtaaacgccgcctctttcggcggtttttttacgtcaatcaacgcagacagagtgagtgaggggagtgcctggctgatatcccatgattcaggctggtatagtttccagccgggttctggtgtttcattaaactcaagaaatgttgtcagagcactcatgatctgacttttgttacctacaacatcgttgacgagaacgttattgacgcaggaaacaaagccataaagatcaccgagttcgcagatagcgtatccgtcaggttcgatgaggggttttatcatcagtgccagtgaccagaatgaggacgcccctgtgggaagatcctcttttctgataaatgccaccatcgttttggtttcattttctgaatcctgatactgacaggagagaaaatattcagcgtcgttttttcttgcgaatgtacgcgcattcttttcttctgaaggaagcgttttccagtccagaccggagaaaaaagcacgcttgtttttaataaccgaagtgaaattccggttattctgattattctcagacaaagtgacctccaaaaaaactccctgtgttttttcagggagttttcagttaattagttcacattttttattactgcactggagccggaattaagcacatttggtgtaatcaaaacgaccagaatcgtgtcgccgtcttcgctgttaacgccacccccaagtccaaagaaggagggggttattacaccttgcttactggttttccggtttgattgctggaatccactcagaatgactgtctggcctgatttcagatcaatagtctgattaattgttttcaaatccacattcggtgtttgtgctttactgccgccactttcgaacacctcaaaagtaggtttgtcagacagactcattgagaacagcaattgcaggttttgtgaatccggctgaatgtatggaagcagagtcatattgaatccggttgttattgttgccgcattcagtgacgtggatgatcccacattggcggtagaatcggttgtgacactttcaatgaagccctgctgattggcaatttgcatagggacaggcgtcaggtttttagtcacagcggagttttgcgttacgacactgacgctgccttgtgacgacagggctttcagaaatgcttttgagccagccagcttaccgtccacgatggatacaccactggtaataacattctcggcggcatcaccaaatgagccaccaagactcagaccaagatgaccatcattaaatactgcgttccagtcgattcccgcctgttcctggcttgatttcttgatactcaggatttcaacgttcagcacaacctgacgactcatttcattattgcgcttgttcacaatctcctgaacgctgtccagtacatccggggtatctgttactgtcagtgaacctgtggacagatacatgcgccctgtacctggtgtcagcattgaggaaacttcgcttttcaggtcgttgtacagcgatgatttcatttccacggttgtagtttgcgtatttgatgcgtcgccagtcataccgccagaacttgtagagccggtactgccactggaagacattgtgccgctcacaacttttgagttgtatgccacattgctgtccatatatgtaatcgggaatgagcgcgtttccagatagaagaatgtgatacgacctttatcgtatttccaggatattcccagccgtgaagatacggtttgcagtagggcaggaagcccggagatattcagttcagggagtgatgtaccgccagaagagacttttactggctgactgcctactgacgccagaggaaccattccatttgcatcaggtgctggaattgagcctgtcatttgttgtgttgagccagtgcctgtaccgttcagataggtccagacatcggcgctgattcgtaccgggatatgacaggtttgtgtgatgcgttgtgcgatctggcgtagctcaatatctgaacgagtattaatcacaatcgggcagggcggcgctgattgctttacctgcttttcaataattggcgttgtattaatccattgttttgttgaatcctggataaccgggttattacgaatttttttcgcgtattcagtcgctttttgtgcgtctctttcggcagcaagttcattagcattgatttttttgaagtttgcacagcctgataataaaaaagaggatattaataaacaaagcaattttttattcatgtttaattaccttatggaatatgcaaaatactatttgctggttgcgaggattattcagatacgtctgttttatcgtgattatcaaggtctggattagtaatcacattgcctccggcaaccatgactttttttcctccagctttagggagataaatttctgcaatttttgctatttcatccatgacttcttgtgtaaacaggtttggaatgtcaacatgattgttacgggcgatatccacgcggcgtttaaactctggttcttcaaaccagattattttgttggcaataaatggtcggctaaattcactggttataatttcacggattgcaatatttctggctttattcttcttatcccggagagacacaatttcttcaggaagaagaaccgggcgctctattacatcatcattccgtgtacgtgaacttcctcccttaccaccaccactggatattgagcgtttggatattttcatatcacgcaccccaatctcttctgaaatctttttagccagagcattttttgatttcggcggatagtaaagcacaacagcactgttttcagtgaatgtggtccatccttcctgaccgtacatatcgcttttttgcccttgtccttcattctgaagaatgaacataaagcgaaggttataacctgctgtaaaaccaacagcgcgttcaatgacctccgatttacccattgatgtaaattcatccaggagaataaggcactggtatttcaggtccgggttatgttccggcagttcacgacagttttcattaaccagtaatgagaagaaaaggtttacaattttctcatgagtgattaaggcatccggtgtcagaccaagataaattgacatgggttttttacgaatatcccggatatcaaaatcactgaaattcgtcgcttcagcagtaacagggttgctgaatatattcagtggtgatgaaaagttcgtttttatacttccccttgtgcgatctggtgctgacatgaactcaaagaagaatgattttgttttatcgctaatccagctacgattttcaacttcctgtcccatccatgcggcgagatcttttccattatcgggaatcgacgttttcagtatggcgctgatggaaacaaggacgtccggtgcattatgacctttggctttctggtccaggtgaaaacgctccttgtccagcaggtaaagccctaacccgacaaaaaggttgcgtgcagaatcagaccatataggatcatcacttgccgggataaggattgcggcaatttttgccaggtccgtttcacgaagtaaatcactgcgactgacgcaatccagaggattccagcggtgagagcgtatctgtcctttaatggcctggtcgatagtttccgcgtatccggcgggcgcaaagagaaagcatttttgccctaattctttttgcctgaatccggctgataaaaaccagttttcaagtttgatatccagtatgaccatacttcctggatagtttacgcagttggggataacaatccccacccctttaccagaacgtgtcggggcataaagaattaaaaattgttgtccggcgaaataaataaattgttttttgtatctccccttgaacattttaccgatgagtattggtggatgtttgtaaggtgattttttatccgggaaaaaacctgatttactcaggtccatatcagttgcgagccgggcgtcaccgtagatgacttttttaggcataatcccaatcacgacaattaacatgaatatggcaaagaatattaatggaacgctgaatccaaaccatgccataaaaacgagttttttatattgtgggttgccagaaaatgttgagattacagaatggaatgtatcccacttgagcaaagacatatcaacggatgaatatcttaatgctgtgtatccaccgagatagtttgaggctataagcacacccacaagaagcagtaacaggaagagaattaacccccccgtttttttgcatccatagttatctccagaatttacattgtgctcaaaaagcgatttttttcttcgggttcgtaatacagttcagtcattctggtatggttgaaaaaaagaatgacatcaattgatgtttttatcgtgcgcatgatgaggtccatatcaacagtcataccaacatcagattgttttacaatacttgccagtcgtgaaaatgaggcataagtattattagcatgaatggtactgattgagccttcatgccctgtattaagcgcctccagataactccatgcttcgttacccctaagttcagcaagaaagatgtgatctggctttaatcgcatacacgcttcaatgatttccttcgcttcaacggtattttttttgtagaaaagccggatgtggtttggatgcagcggcagagacatttccggtacatcttcaacggttatataacgccgttcaggtggaaagatatctgcaattgcctttgctatagttgtcttaccggagccagtcccacctacgattaaaaaattaagacggtctttaactgcctctcttaaaaattgagctttagattgaccatctggacgccgggataattcatacaggtagcgttgccgttcagtcaggatggcttcgtgtttcgtcgcaatcctgacattatcaaaacgccctgttcggacataatcatctatagtaaagcgcgttaatgatggcttacggatagatataacaacgctgttattttctgttgcaggtggaatgatgatttgtccacgttccccaccgggaagaactaccgatgcaatgggattatcatggctaagtggtatttttatctttgataagttcgtcagagttttggcaagtgtcattaaattatcaaaagtcgcgtctggcgcatctttagattcccagccatttttcccttcaaaccatattgttcctggtttattcacagatatttctgtgacatgttcaatattaagaacagactggatacccgtcatatccagatatcgtcttgcggttttactgttatctcgtggttgtgatgtattactggtatctctgatgttattcatatcaattgctttactgaattactgaagttcatatacggatgaaaagtcgatatttcgcggaacaataacagacaatacagtcccctgattaatatatgccgtggggggaatatttatcgtattgtccagcgttgtttttgccatttcttttgttgcgtcagatgtgttttcgtaggtgatattgctgtttgagcctgtttgttgcgtgctgtttttcagaatatctaaaccatcaccaagtaatgacagcagcaatgcaccaccaaaacgctgaccaaaatgattatcgacccatgcaggaaggccggaagcgcccagactgtcagtgccaagtgcgccaatgcgcacattgacattttcatctttgagcgttgtccagttaacgaacacacgggctactccctgtgtcatgactttattctgctccccgatcagtaatgctcctttacgggcaagaagcacttcgccgttatccgaccagacgtctcgtgttaactgacacattgtgataccgggataagaggttacgattttcgttttgagcgcacaggataatgctgttcccgcagataacagatatcggcggttcagcacaggggatacctttccgtctgcgtactgactgccctgtaaatcatctctttcctggtttttttcgcctggttcctgattgtcgatcttcaccattgtgtcgcccatcagttgtcggacggattttggcaatggttgtggttggtttggatctccactggcggtttggggagtgccgttggctatcacattgacggctgtttgatcggccttttgtgatgctggcgctgacggggcattgtctgcctgtttctgttcttgctcctcctgtgctttcctgcggttagcggcatcgagtttttccttttcttcgatgtttttcatcattgttgtcagtgaaacgccattgttggtatttgtgaccagtgtctcgtcagacttctcaagaggtgtttgctgaacgacctgttctcgtgaaagaactttaaacaagataatgatcacaaataacgcggcaatcagtaatatgatgaggattaatatttttttgggcgctgctttaccttttacttcgataattccgcgatttcccgcgtcattatttttttcatcatctttatttttcataactgaacttctctactcgtgtttttccgtttgacgtaccttttttattccacggacgattaacaattgatgatgttctgatatctgcaacctgatcacccaggcgcaaacggagattttcagccacgtcatagtaaaccattgtgtttttatttctgtctccgttaaccagatgttcatttccatcggcatcaacacgataaaggaccggaatttcctgtttattagtccatctcatacatgtaaattcgccattatcccatacctgataagggaaaattgatttgtcccccttgagttgataatgaccattaattattccaccatcagtgcatggaaatggcctttcagtcattacggtagctgacggtggtttcggataattaaaacgcagaatatacgtgggttgttttgtggtcgatatcaggtagagaggataaatatgtttatttgtctgaacggtaacgtttgtatccggttcttctgcttttggacgcagaaacagagtgttgtccctgaccgatactgaccaggcttctggatcaccaattccgacatcttttattgtttcgtcttgtccgaacttaattgttgtttgtgcgcctgcttttacgcgcacattaaatacatcctgctcgttataatctacggtctgaatacgcccgtctcgttccgatggtgtgccgtacatagctgctaaagcaccaggtacttgtagcatcgacaaaagaactatcgcagtagtgcgtttaagcatcattttataacctctggatcaggttggtagctcagaacttgcagtcccagcggattcacaaggcgctctttttctgttttgatatctttatcccagtcaaaagaaatcgtcgaaatccactctgttgctctgtatccgggggcgggtttaccagataaatccagtaccatttttttgaaacgcacctgcgccatatccttccttagaaggattactgaattaatctggaccttaattttgtagttgtttttcagaatgttcagtggtgaggaattagcacgaatcatggtgtcataagctgaaaacaccttttgggaagacattgtttttactgtatcggcctgttcctggatagtctgccagtcgtatgcttcatagttaatgagatattttgacaggaaatatttagtctcaacatcctgataactttccttagcatcagataattgaggggcaatatcagtatatccagtgctgttatccactcgcacaaggaacggtacagcggtttttaatggagcaagggaggctattgcgaaaccaagaatgcaaattacaacaaattcaccaatggcgagaatggtaaatgtttttgcctttttctttatgatttcaatttcactgcgctcaaagtcacgtatggccttgaaataatcctttttttcgttcttttcttcaaatttctttttggaggcttctttttttgaatgttcataatagtttgtgttttccattaataatcacctgctttgatttgtccaattgctgtgtttaactcaaaccatcccccttttttctgcacaggattgagggtgtgagaacatgcactaattaatagcgcggaggcaattatgaggggggcatattgttttccgttctgtataaaacagagaaattaaccgttcaggcgtttcaggagttctggtatccagatatcaggagctacatctcctttttcagcaataatctcatcaaaaatagacaggccagtttcagagccagaaagcacgggcataaactcatcgaatccaaacaaatccattttggcgaaaacagaagagccagattgcttgatgagcatggtccttgagtccttcgccagctctttgagtttttcaaactctttttcagtaagcccaatttctttatatccttcccattttgcatcgggattgggcaggagaattttcgttgcggtttgttgtaccagcgccgcaaaaattgcgcagtttatggcgtcttccggggactgtgatgttaaccacatcatttcacctttcatgcgccccgctttcagtgcgcttttaatcatgctttgtgtcatgggaaagtttgctggcatccagaactcttcaataatggagagcatcagattgccgccgcgttgcatgatttctttgtagaaaaacaggaccgcgagtaaaggttcacaggcaggatgaataccgttttttgtgtccaggacaacggttgtatcaaatcccacttttttatgatgcagtggattgaaggtattccggggggaatccaccgcccaggcatattcgccgttgtcgcaccatttggcgagacgcgtctgaagttcaggactgactatatcgagaagaagggctgtccggcgttcttcgacaggcattcgcataatgctttccacggcagcgtttaattcaatgccatgttcatcagaacaggggttgccctggttgtctcgtgccagaacctgtgtccagcgtttcagaaatgcgagaagtctgtgccagacgggagattccgggcctgctgccagttgccacggattgcagcctgtataaatgccttcctgcaacgtgaaatagcttcctccgtatgcacggacaaaaagctcagtagaacggttgaaatccacaacaaacattaaagggtcaaagcgttgaagaaaaccgctggctgctgcttcaaatgttgttttccctgtgcctgttgcgccaagaatcattccatgcccggcaattttttgtccggtaacatttttttccggtgggctgtaatgcgtattgaaccagtaaatgctgtcagacaccgttttcaacggcattatggcggacccatcaccaataggattgcctgattttttcccggaggaataattgtgaaaagacatcagacaggccaggttggttattgttcttcgggtatccagtggacgcctcttattcagcggcatatgactgaaaaaaacaaaaggtgatgcgagtgaggcgcgactgaagcggaatcccttacccgaagtaataaactcagcagagagctttataccattgcttctggcctgatctggtgtatcaccgaataccgttaatgcgcagtgtagtgaaccaaacaacgtaattccggctgcgaccgcttctttaccagcttcgagttcttcctgttgtgttttggctgcatcattagctgattgcagtttgtttaattgcgaatcaatgttcttcagggtttttgtgggtgattcaaaaataaacgattgagtaagaataaattcatagggttgcttgagaagaaaatcccattgtccgggggtcgtttctatagggaaatccttcaaaatataattcgttgcaaactttttactatccgattcgttgttacgcagttcaagaacatcagcaccaaaataccattcagaatcacaaatactggatgataagggagtggatgataaaggaatcatgttatgttctgtatttagcaatagtgataaatagtctgccacctcactaatataagtattgtgaactgataaaacagaagcgttaaatggctgaagtgctttttgcgcctgttgtgtcatttcattcatgcgctcgattccggtatcaacatcatcgtagggaatcccaaatgttaaataccatgttgatttgtagaatgcgctggatgtgaacagagcaaggtatttttttgaaaacctgcttaaaaaagcattgtcaaaattccattctccatcaatggttattctttttttgataagatgagtccataaatacaggtcgccttcctttcctaacccaatcaggaagtttttaacgctgttaaataaatttgtaagaacattatcgttctctgtctcaaatggaatgcctgctacaacaagtgaagccagcattttcctgtcagaagtaaagataatactgtctgttatatggaagcgataatcgggatattcacgagcaacatccatattgtcactgttttttaatttgattttgtgcatatatgttacctgacagatgaataaccaataattaaatctctgtggcggatttttaacaaaagccccataaggtttaaacggatgactcttagtgcgtttgagtcattttcacaggccagttttactataaaaagaaacagcgcacatatgacaggaataattatcccttttataccccaaaggaaaatagcggggaagcctgtcagaacagcgaaaaaagccgtgaacaataacaacattaagggtataccagcaattaacgcaggacggttaaacccgttataagtcatgaatgactcactgacatgttcatttactttgtccataactcaccatgaaaaaagccgcaattcacacataaggtaaatatgcggcacagttgtatatatggattacttatttgaagagttccagcgcccagttcccggcgtaaagtgcaccgccagcgagggaacaatagataagcgccattccaacatcagaccagcttttcttctccatgaatgccatgatgatgttatagatcaggtaacacaaagcacaaacaccaccaaagccaaatgcccatgtcttaagttctgtcatggtatttgttgcggtgtcgagtccacctgcataggcaacggaggcggttagtgacaggaaaaacgccaggataaaatactgcaaagttgatttgactttcattaactaaaatctccaaaaacatcccagtccggtgatgttttctcttcatcattcgggggttttatttctgattcatcagttttttcacggattgatggcacgatatagttattcgcgttactaataacccgttccagataagaagtgttgttaaatttcttttcttttataaatcctgttgattcgttacccgaataatacaaagataaagcgcggagaattttttgattactactaccctccttttgtctcagataattgtccgtcaggactgccgctcctgcttttatatttgtacacgggtcaaaaactgttttattagtcagattgagtgatgggaaatttttgctgtatatctgtgtcagacctgcactaaaccttttattttctgctgttaatttttctgcatattcaatagctcctttttcatctttaaaatacttacttgttccgtcagaaacattcgctattacatatggattagcccctgattcagtcataatcagggcattcatcgtatcgggtgatacatcaggggcacactgacttatcagtgctgccagaacagccgctgataactgcataagggatttatctcctgatattaaattcgtgaagaacgttttttcctgcgggggcgacgaacgatactgtccagctcattagcgccggaaagaagttctttcagcgagacaattttttccctgacaggtataagggattttgcccccggacattgttcaaagtctgatttgtacaggtcgtataaaacaagcgtatgttttacatcctgtgacagataattatgtttattctggatgttaccttgttcgatatttttgataacatctgctgaacgatgaattaaatctgtgtagattcgggtgtttttaaggggatttatttttatatattcagaagcaagctgaataagattgcatcgtaagataaggacttcacgagtggcaatatcaagctcgctgacatcatcattatcctgcatgggtatctccggtaaagattttctggactgaagtccgcgccgtagcgatatatccggcccacataatgatgtgtcggatatgcagactccagttcagaatccccttgtcaggggatatttagttaatgagtacaatagcctcttctggaattgaggaaggaacgggaacgcccatatcattcccctgattatcgacaatcttcttattttttactcttcctaacattgctgaatgttttgtttgctgctgcaaagcagacatcagaccttctgtatcggcagaccagatataaactcgcttaccgtaaataatatgatgaatatcataaacgggtgttataccgatttgctcagatgttaacgttcctcctgaattacgtttgtctggatatttataaagatagtcattgataacatttgcgaaatggacaaattcaattgcttcctgttttgcaggaagcaacttcattcgctgtatggtttggttattgttatctgtcagatagtaactgcctattgacaggaatatcaggagcaagctgaagaaccaacccatagcgattactcctgttgtgtgtctttaattgtgattacgcattgtgcaatattcctgaatgcgtataacggtttatctgctgaaagataaagcatcagcgtatcttttaatgcagattcaaagtttccccggaaaataagaggagcatctattctgtagttcacaggagtatcccatacaagattccaggttgaattaccacacggcgaacttgacgcccattctgtgaggccatcccgcagcgtcgaaccttttttaagattccaggtgagtttttctgtctgtttttgcggaacggtgacaggcgttagcgatttttgaggtaacttgtcagaggaaactggtgtcctgacaggagatgcaggggttttgtcaggagatacaggtgctttttgtgatgttttccctttctgttgttgaaaatcaatcagaagatgtcttttttcggtgttcttaattaccgtcagaccgtagttgcgaagcattttatccaggacataaggccattgatcgttgccctgccaggatatcgtgcgacggaaagttttcgttacctccggggatatttcagccgtccagtccgatgggacaatttcttttactgctttttgcagcgagcctttaccatcatggcgaacaaagtaaatttgcttttcaggtgttgatgggcctgtaagcgtggtggccggaacagcgttgttagcgttttttttcagaaaaccgctttttgacattagcggtggttgttcaggacgcggttttgtctgctgatcagtattttgtgccgtttgtgcctgatggttgttgctactgattggctttgcgggagggagtgctggttgttcagaatgctgtttagttgctcctttggctgctgtttttatctcaccagatgaagttatgctgtttgttgttggcttcagaaaactattattgctgtcctggttttttggcgctgtcgctgattgctcagaacgtggttttacggtttctgcaatacgaggcgcgggagttacgtttatggcttcctttttgagataattatcattgctgtattgaacagtgctgtttggctgtgacacgggctgctgaaggatcaccccgttatagtatgtgtctgctcttacctgctcaattgtggtattcagttcataccaacctccttttgcctgtgcgggttcaggggcatgggaacacgcattcagcaataatgtggtgggaataataatgagctttttcatggttaaccccaggaacgtccgaacttactcatgatttttgagatcgccgcagggtctgcggtcgtgcattgattaagaaacgcttttcgggcatcaaaggttttgctccagcgaatagatccctttttctttttgatgatattaaagaagtccttttcggcgcttttgcattcgctcccgccgccgctaccgatagctttaccgtacagacaaagaacggaagcacacggatcagacgcatatacgggggcagagagtgcgatagcacttgcggcaatcatcgataacatgattttgcttaacggtgtctcaggcacggggtgtgttctactatctgcgcagcctggtcgtaatgctgcggtggcggcaaccatcaataaaatggtggatttcatggtgttcctccatatttcagacaaaagaaaacccgcacgaggcgggtaatttaaatatcaggggaatcaacgcacgtgtggcgtaatgatgacaatccggttctcactttggctatcagcattaactccagtaaaaccactcacgataaggggctggtccggtttgagtaaaatattctgctcgacagtctgtttccgtatcgggtcagtggttgtgagtagcaggtggaaatttccacgcacaatctccggtgctatccgcattgtgaagccagcagtccgatctttttcagccagtgtagtgtcaccggatattgcctgacgggggatcagttccgtggcaggttcccagcttccggcagatgttgtttgtgattcaacgcacggggtactctgcattattttgcggattgtttcggaggatatcgcctgaccgtcatcaaagaatgcactacggaaaatcacgttctgatttttggcaaacaagcctggatgccgctcgctcagactttcgtatatattcataccattcaaacaaacaggagaactgtccggcaggtctgttcgcacggtatcaattttcaacgttatctggtggtcatttgaggttaacggtgactcaggtactggttgtgttttgctatccgcgcagcctgacagtaatgcggtggcggcaaccatcgataaaatgatggatttcatggagttcctccatatttcaggcaaaagaaaacccgcacgaggcgggctttgtgacggggggagaggagaacgttattgtagacgttccattaattcatttactttcttcttgtgctcttctttttccttgtaggattgtccaagcatctgatttataatttcttttctcttgatatcatacagtgcttcgttggcattcttacagttattatttgtaatttcaccagctttgcatttatcagatatcttttgggcttcatcctggtgttctttatagtaagagacatcatagattttgtcctcacatccagttagcaagccacagaataaagctagagaaatgatagtacctttattcatggtatgtattccttaatcaaggcgtcttctcatttcactaactttctttttatattcactatcttgtttttgctgaagtgcagcacgagcattatcgcagttttgatcggttattgtacctttcttgcaatcttctaaagttttagtggcttcactgatgttgtttgagtaatattctacagaatatattttttctttacatcccactaacattaaactggcgaagataattgatgaggcgataattgcatttcgtgacttattcatatcagcttaaatctcttctcttttggcgaacatatgtttcatattgagctttccgctcattctctagttgccgttcagcaatatcatattgcttcaattgtagctccattgtcaatttttctgtattcaattggttagataagtcagcttttttttgcggagtatctgctgacgcgaactccttctgtaacgtggttaatctctcagccctttcatgcatttcattgttgaaattttcatagaatttgatcttagccaggattgcatcatatttttgttgcgtctctgcaacgttgcttgataagtcatattgcgatcgcaaagagctaagatctgattttgcactatttgttattgttgatagagaatccttcatatagctggatgcgctatcaaagccattgctgaaatcagtgaagccttcgagtcttttcttctgttcttcagcgtatcttatggtctgttcatattgttgcttgagttgctcaagctgtcttgccgcttcagcggcacgatttagcccttcttcaactgttttagctatcgccgtaacgtctactactggaataccagacgcaaagattgatggtgatgttatggtaattattccagcaaagagaaatgataatactttatctttcatgttaatcaccccgctttaagtttacttccacggtttaatccctgaactaatgctgatgcagaacgacttcctgcattaaagaatggatttggcattttgctggcaaaaccattggtgaaagatcggagtcctgatgctcgcataaaaccggatgcaaccttacctccaaagccattagcggctgatgtaaggccatttatacctaccccgccagtaagagttgagcagagagtacctacttgctcaacaaggaatatagatataacagtcaccataaagaaataaactactgttgtgagagttatatttccgcttgatgggactgtttgctctacaaagctgatgacgaagctgaacgatatactataaaaaagatttagcaatatataatttaaacagctacctacccatgcgctaaacatatttcttgtcgaagcaaataaagaaaaacagataaataatattccggcggataagagaatgccaaccatgaatgttgatagggttagaaatacagtggtgtaataaataagcagcagcccgccaacatatccaaccccccaaattagatatatgtatagttgatctgtgaagctataccactccagagtatctaactcattattcttaaaatcatttaacgttactgaaagcatattccacaaattatcaacactggttgcagttcctgaacctccagtaacagcagcagaaaggtcggagccagcgtgcattacaaaaggaataacaaactggctgtaataaggcgctgaataagtgaatgcaccaaccagggcgaatgcaccgatattcttagtcacctcgctcatcaggacgtcacgttgggcatacgtgatttcatatattagataaataacgtataaggctatagccgcaaaaaatgtaggagatattgcctgtgcgaccttagcggcgttcgtcgccgctgtggattcaacaatggttgttactttctcaagaaattctgaaacaatgttcatagcttagtccttctggcagatgaagagttcaccagaggcactaatatttggcttcagcttaattaccaggtctgtgccgaaaaaaattgcttctccaattttttggaaatttaatgtggattcttctttattgtaactataaacgttgtgggtcgctgtcgatgttttggttttacgtggtgtaaacgcttttttaatgatgtaatcgagagcgtggctgttatgagatggtaaagtgattaaatcgtcgttttttttcataatgtagttctgatttttgaggttgaagataattgtcggtcaatgcttttgattttttttcgtatctcaattggattcgcctcagataagttagcaatggataaagccagttcgatttgtaaaattttccttgtttcgatgagattgatttttacttgtggtatatcacggaagacaccacagcctgtaattgcttttgaaacgacacgttctgtattcgctatgcttttctttactgttcgaggggggacataacgtgcacggcgaagttttctcagattagcttttgcttgagtcatatttgtatgccatgaataaataaatgctttcttcaaagcctgaataagcataaaccagtaatatggtggctaaaggcaaagaaaaggtgaaatgaataacggcattcagaactgaaccggttaatgtatataagacaaacggtgctgaaagtatgaaagcacccagcaggaaagaaaaaacattgcacgccatttttaataaaaatatttttattaggtgatgtttttttctgtacatttcatcgtatccttcagggggctgtcaccttctcacgttgttgcttccatcgcgagagcttcaacaagttcattgatacaagtccgttctgccggaacttcccgacctgtaaccgggtcgaaaacgatatgctcattttcaacgtaacgcaggcgatcgtagattttttgacgagcttttatcttaatcacgtccagctctgcatgttttaacttttctcgtctatcgccgatgaaaaatattatccatatgacagccatgataattgccgtacaggggagcatgtagcttaatacttttacagccacagcaaatgtatctttggcccttcctgctgctgattcaaggataaaaaaagctacagttattattatgactgtgtatgtctcgtatttatttacacctctaataccaaatttgcgtaatgtggctttagttacaatctctgaaagagaaacaccttcatttgcgtttcgcattgaattttcctcatactattcctgtgacacttaacaccatcaataaaatatgtaatgcagtaatgttttttagcgtttttttgtaggcttcaataaaatccttatccagaagtctgataataaaaatacatagtgcattcatcgctgctcctgtaagcggaattaacaacgaaagatgcatgatttattccttgttttcactcttgtcttcgatggtcattactttaagagttattccttgcatgtctgtaacaataactcttgttcccgcagggtagttttcattgtgtaacgtctcaacaggccagaatgtatcaccaatgagtaattgccccttgccgttaacaatatcgtcactgagcgtaaattcttttcctttaaaacgtgaatgccctgtgttcaccaggagagtgtctaatttcttatgttttttgtcatagaagaacttaattatcgacgccagtatgctgattgacgcaaagaaacatatattggcttcttgtgatatcggaagaaaaaagcccataatggcgcttgataatgcaccaagactgattaagagtaaccatcccgttccggttataatttcaagtgcaatacacagaagaaacagtgtaaaccataacatggctaatctccatgataaaaggcggagagtgaataaagataataaaggtggtaacaaacggctaatacattaaacaacagacataaaccgaagaatatgccattcaaactccatataagcagaggcatcgttgctatcatgagcgtcgaagatccccagtctagccatcttggaaatatggagtcgataatatgctgtagtctttctctttcattattatctcttgtaatagaaaggagaaaaaaccatgttgacatgggaattatatacaaaatgataatgatgcatgtataagttttcattttaatggctcactcaggagttaagataaggacggtgattaaaataatattgtcaccgtccgctatcttttttaagacaaatattgttgagcctgtttcatatcaactttgggagtatcctgtgatacgttttttagcagttcactgatacctgcaaccgaactcaccaggctacttgaatcaagcggcatcataaccagtttactgtttgatgctgtgccaattgcctggagtgcttcagtgtatttctgcgcaataaagtaattaacagactgaacatcaccttcagcgattgcatccgacaccagttttgttgcgcgagcttccgcttctgcctgacgttcgcgtgcttcagactgaagaaatgctgattgccgttccccttccgctttcagtatttgagactgtttttccccttctgccttcagaatttctgactggcgtataccttccgcttcaagaatccgggcacgcttagttcgttccgctttcatttgcgcgttcatcgcttcagtcagttcttttggtggtttaacatcccgaatttcaatacgtgtgaccttaattccccacgggtcagtcgcataatcgacaaccgttaacagtttagaattgatgctgtcacgttgagaaagcatatcgtcgagattcattccaccaacgacagttcggatattggtcataacaagatttgaaatagctgatgcaagattatcgacttcataagctgccttcgctgcatcaatgacctggacaaaacaaacagcatcgatagttacgtttgcattgtctttcgaaataacttcctgtttaggaatatccaggacggtttccatcatgttgatccgttgacctatgcggtccataaacgggatcaggaagtgaagaccgggggaaagcgtatgggtatatttaccaaagcgttccactgtccaggcattaccttgtggcacgattttgacggcagatttaacgaatatcagcgcaactaaaattaataagagtaacggaatagatgtaactattggcgtaattatagcgtcaatcatgattaatcctctcccaattcatcactgtggatgtatttctttgcaaatgaaatctgaaatatgaacatcaacagaaaaacactttcctgcttctttttcttttgagaagttttttctgcattcttcagcggtc

SEQ ID NO: 4 - DNA, 12908 bp, Salmonella enterica
  source 1..12908: mol_type = genomic DNA
attttgaaagcttgatcggatatgacggctgcgaaaaattggaataattgcaggttattttgaatatgggggatttctaaaacaccccattaacaatgtgttgacaattgattttgaccaagttgttaatgtttttcttgcgttttcaattggttgattagataatattatatagatctataatctattctagttgggttgggttgggttgggttgggttgggttgggttggggaaattgttcgatcggaatatatagtttctggtgttctgatagccataagtcaggggcggtagcgtggatttttattgcgtaaaaacgtcagtaaacatagaggtttaatgaaaaaaatactattaatatccatgagtttgggaaagggcgattatggtggtggtatagtatcaaatacaaacttttttgctttaaaggaattagagcattatgagttattttcagtaggtattgtaaaaaacaccaacgatgcaccaaactttattaacatggttttgcctggcaacgcgagtaaattctcaactgcaattaataatatattagggtttgccgggcagttaaataataaaactacgaaaaacataaaatacataattgacgagtttgagcctgatattgtataccttgattcaagccttctggggtgtatagcttcttattgcaagaaaaaacataaaactattcaaataataacgttctttcataatattgagtttgattttgaaattgcacgtattatgtcagggttattgcatttttttccctcgttaatatccacaactctcgcagaatatgcggctgtaaggtatagtgacaagattatcgcattacataaaaaagattcttttagactagaggaaaaatatggaaggaaggctgattatatcgtacccgtttgcattaaagatacacaaagagaaaaaagttttaaactcgtcaacgaaaagaaaaaagaaggcaaaaagttaaaagttggctttataggtacagctttttttgcaaatgtagagtctgctaagattatatctcaatacattgcgccaaaggtggaggggattgctaatttttatatatgtggcaatggatttgaaaaatataaagcattgaattcaactaatgtaaatgtttcaggctatattgattctttggatgatttctataatgaaatggatgtgatgatatttcctatttttagcggcgcaggaatgaaggtgaaaatagcagagtcattaatgtataataaaccaattctagcttctgcatttgcattagttggttacgaaaaaataatagatggaactaatgtgattagctgtgagtctcacgaatcttttgtttatcatataaaacaatttagaagggataataacacattatataataggaagtcttattataaatatttctctgataaagcatgtctgcattattttagaaatatattacgagaaattgttaataacgaatagatagatggactaataacgtcatgaatataatacaattatcaaaattctaccctccgatatttggtggaatagagcaagttgcacaggacataaccgaagggatgcaaaatttgtgcaatgttgatgttttaagcgtaaataacagttctaaaacaatatattgtaagaatataattcgagcttcattactatttacactattatctaccccagtatcgattagctatatactaatctggagtagaatcagaaataattatgatataattcatgttcatttgccaaatcctcttgctattattgcactgttgctgttcccgcccaaagctcccgtcgtagtgcattggcatagcgatatagtaaaacagaaaatagcgttaaagttttttcggcctttacagaatttatttttgaataaagtagaaaaaattattgtgacatctgaaatttatgggagttcttctcctcaattacaaagatttcaagataaaataatttgtataccgatcggtataaaatcagagcgattacctaagaatgaaacgctattaaagcatctaaaagaaaaatataaaaataaaaagatagttttctctcttgggaggctggtttattacaaagggtttgaaaatctagttaatgcagctaactttcttcctgaagatacaataatccttattggtgggtgcggtgagttatatgacgagttggctgatagcattttatccaataaactagaagggaaagtggttttgctgggggagataaaatatgaacagctttctgcttattatcaggtttgtgacgtattttgtcttccttccattcatgaatcagaagcttttggcgtggttcaaattgaggcaatgagctatggaaaacctgttgtatccacaaatattaaaaatagtggggtcccatgggtaaatgaaaatggtatttcaggtgttgtagttgaacctaatgagcctcatgaactagccaaagctatcttgacaattttaaataatccagctggtttttccttaggggcattagagcgatatcgcaaattattcactagagataaaatgatctcaagtttgattggactttatcagaatattaaaataggaaatgaaaaagaatgatcttacctgttattctggccggtggtgcaggtagtcgcctttggccactttcccgcttactttaccccaaacagtttttatgtctgaaaggcgagctttctatgctgcaaacaactatctgccgcctgaatggtttggggtgcgaaaatccggtggtgatttgtaatgagcagcaccgctttattgttgcggaacagttgtatcaactgaacaaactcaccgagaacattattcttgaaccggcaggacgtaatactgcacctgctattgcgctggcggcgctagcaacacaatggcataaaccagatagtgatccgttactgttggtgctggcagcggatcatgtgattgctgatgaagacgcattccgcgccgctgtgagtaatgctatgccgtatgccgaagcgggcaagttggtgacttttggcattgtgccaaggcatccagaaacaggttatgggtatattcgtcgtggtgcagtgtctgcgggcgagcaggagggagtagcctttgaagtggcacagtttgtcgaaaaaccgaatctggaaaccgcgcaggcctatgtggcaagcggcgaatactactggaacagcggtatgttcctgttccgcgccggacgctatctcgaagaactgaaaaagtatcgtccggatattctcgacgcctgtaaaaaagcgatgagcgccgttgagccagatcttaattttattcgtgtagatgaagaagcgtttctcgcctgtccggaagagtcggttgattacgcggtaatggaacgaacagcggatgctgttgtagtaccgatggatgcgggctggagtgatgtgggctcttgggcttcgttatgggagagctgcactcacacagctgaaggcaacgtttgccgcggtgacgttattaattataaatccgaaaacagttatgtgtatgccgaatcgggcctggtcaccaccgtcggggtgaaagatttggtggtagtgcagaccaaagatgcggtgttaatagccgatcgcagctcggttcaagatgtgaaacatgtggttgagcagctcaaagccgacggtcgacacgagtatcggatgcatcgcgaagtataccgtccgtgggggcaatatgactctatcgatgcaggcgctcgctaccaggtgaaacgcataactgtgaaaccgggcgagggcttgtcggtacagatgcatcatcaccgcgcagaacactgggtggtggttgcgggaacggcaaaagtcactattaatggtgacatcaaactgcttggtgaaaatgaatccatctatattccactgggggcgacacattgtttggaaaatccaggaaaaatacctttagaaataattgaggttcgctcaggttcttatcttgaagaagatgatatagttaagttttatgatcgttatgggcgagattaatttcattactgaagttgctaattgtttaacaaaatacttctgtgtattttttcatataagtttgtaaatatgactagattaagctgctttaaagcctatgatattcgcgggaaattaggcgaagaactgaatgaagatatcgcctggcgcattggtcgcgcctatggcgaatttcttaaaccgaaaaccattgttttaggcggtgatgtccgcctcaccagcgaaaccttaaaacgggcgctggcgaaaggtttacaggatgcgggcgtcgacgtgctggatatcggtatgtccggcacagaagagatctatttcgccacgttccatctcggcgtggatggcggcattgaagttaccgccagccataacccgatggattataacggcatgaagctggtgcgtgagggggcccgcccgatcagcggcgataccggactgcgcgatgtccagcgtctggcagaagccaacgactttcctcccgtcgatgaaaccaaacgcggtcgctatcagcaaatcaacctgcgtgatgcatacgttgatcacctgttcggttatatcaatgtcaaaaaccttactccgctgaaacttgtgatcaactccggaaacggcgcagcaggtccggtggtggacgccatcgaagcccgctttaaagccctcggcgcaccggtggaattaatcaaagtgcacaacacgccggacggcaatttccccaacggtattcctaacccgctgctgccggaatgccgcgacgacacccgcaatgcggtcatcaaacacggcgcggatatgggcattgcctttgatggcgattttgaccgctgttttctgtttgacgaaaaagggcagtttatcgagggctactacattgtcggcctgctggcagaagcgttcctcgaaaaaaatcccggcgcgaagatcatccacgatccacgtctctcctggaacaccgttgatgtggtgactgccgcaggcggcaccccggtaatgtcgaaaaccggacacgcctttattaaagaacgtatgcgcaaggaagacgccatctacggtggcgaaatgagcgcccaccattacttccgtgatttcgcttactgcgacagcggcatgatcccgtggctgctggtcgccgaactggtgtgcctgaaaggaaaaacgctgggcgaactggtgcgcgaccggatggcggtgttcccggcaagcggtgagatcaacagcaaactggcgcaacccgttgaggcgattaaccgtgtggaacagcattttagccgtgaggcgctggcggtggatcgcaccgatggcatcagcatgacctttgccaactggcgctttaacctgcgctcctccaacaccgaaccggtggtgcggttgaatgtagaatctcgtggtgatttaggacttgttgaaaagcagacaacatatatcattaacttattaacaaccccccccaatgatgaaaagtgaaaatattacattcgtagtgcaaggtccagtcagaaatgaaacaaaagatactttagaaagtattaggctaaattttaaagaagctaaaataattttgtccacatgggaggggagtaacgttaaaggtctttcatttgacgatattgtttttagtaacgatcctggccctttaaatataaaaagaggtactaaaatagttgctcaggaaaatacaaatcgtcaaataatttcaacataccaaggtttaattaaggttcgaacaccatatgcggttaagttgagaacagatacgccattgcgaaatgataatattataaaaaaatatataaaagctcaagcatatgggagagattataatttttcatacttagaggaaaggattcttgtatcatctataaatactatagatccaaaaagctatattcaattcccataccatatttctgattggatctactttggtaaaactaacgatttattaaaaatttgggatggggaattaatagatgataatgattattttactgagcaggatataaaagaagagattagttgcaaaagaggatttgagtttggaagatacacagctgaacagcttgttctatactcatttttaaaaaaaaataaaattagcgaatataaacattattgtgatacaaataatgacagggttgaaaaggtattaaaagtaatcctttctaatttctattgcgtaagtcctaaacaaatgggactctgttttgataaatactcagacttaataacccctagaatgaattacaggagtctccgtagtttcttagggttttattttgttacaattaatgagagagtttggcgaaattattacagccaattctataaacttgacactacaatatcagaaaaaatagaaatatgtgcacgcaggatatttataaagatcatgaagaagaggaaaaaatgagtaaaagcatgttatatatctacacacataatggttacaatgatgcatttttaaaaaaagatgtagggatggtaccatctgttatgttaaatggtgggaaagttgattcgctaatttattgtaaagaaaagaaaggaaaggctgtttttaataatttagaaatatattttactggatgtggatggtttattagtaacttcaaggcgatactgtttctttgtcgaaatgcaatatttaatagacatttagatttattgatatttcatctcagccttagaaatatcgtgcctgtgttaacatataaacttttatttaaaggggaattaatatgtaagatggatttaaatactgaaagtgccatcgaatattctgataaaagccataaaataaatgggatcaaaaaaaaactaataagaatgattatttcccgcacagatcaattttatgttgagactataagaaattataacataataaataaaggcatttttgatgtgccgatttcaggaaagttaaaattaatgcccaacggagttgatgctaaattagttagggaggttattaaaactgaaggtcctgatgtatttaaagaaaatacaataacaataatctctcgccatgcttgtgaagcaaaggctccaaacagaatattcgatgtaattgacgtaatgtctgaattgaaacttaataactggacattgcaaatattgggctatttcccaacaatatttacggatgctctaagaataagagctcttgaatgtggagtgaatatcatatgtgacggttataatttagagctctatgatgtgtataaaagattatttaaaagtaagatgtttatatgtctttcgctgcaagagtcatattgtatatcactagttgaagctgctgtatttaataatcatataatatctacaaatgttggagtagcagaagatctttcattaaaatacggtaatattaaaattatcgaaaattattcgaaaaatttactatccaaaactattgaaaagacaattaaaaatgatattaaaataaaaaaagcaagcatttctgatagaatgttagactcatattcttgggataatatcataacaggggtcatgagcgaataatatgggagtatatttcattttaatgacagtggcactattgatctctattatcgtattattccacaatgatgttaaatttacgaatgtatatatatatacttatattttactgctctcattacttgctggtttaagatttcataccggctgggattgggaagcttatgactattttttttatgagctgacaagttctaatttatttgaaataagcaaagcaaatgtttttaattatgaaccagggttcgttttactatcttatatatctgttttattgcacataccaccatttctctttttctctttaataaccgttacattaattataaagtcggcttataagtatttagggaactttgtttacatttttttattaatatatttatattacggttattttcataatttttctattgtacgacaaggtgttgctgctgcgttattcgcttattccattcgttatttgatagcgaaaagctataaatattatatattaatatttgtcgctgcattattccatatgtctgctattatgttattaattgtcccatttctttataaaatcgctaatcgtataccattttccgccctattgctattttcattgttaatggtttatacctctgtaagtgagctcataggtatgaaatcattattgtcaggaataaatgcactaaatatatatatcaataatagttctttaagctataaagttggtttttcgtttaaatatttggagttattaatcattctagttctattttataataagagattttcagaactgattgctagcaaatttagtaaaaagcaatattatgtatttagatgtttgattgtaattgaagtgatgatatattcgcttttcaatgattttagcattatttatgaaaggctaactgtttacttcgaattttctcatgctatatgtatagcaatgattatatcagcctttaagtataaaagagttcagttgtttttactgttaattttattgtctcttatttttgtaagatattatcaattattcaattctcctgttcgtgtcgatggagaaatgacacattacgaacgatttgaaaactattgttctgtttttaacacaaaagattgtcaaaggtaacaatgaagcgaattattatagacttggataatactatcacaatccataacgatgaaccatacggtaaaaaaccagtaaatcaagatgttgtaaataaaatgaagatgtataaagagctgggttatacaatcacaatatttaccgcccgtaatatgaatactttttcaggtaacattgggaagataaatgttcatacaatgcctgttataattgactggttaaatcgtaataatattccatatgatgaaattattgtgggtaaaccatggtgtgggacaaatggattttatgttgatgataaggcgattaggccttcagagtttataagcttgtcagaaacggagattgctgaactactggataaggaggtaaaaaaatgattctaataaactctgctgcttacgttaatgacgacctaagagctgaattcggtttgctaccaccgtgctttttaccccttggtaatagaagactatatcatcatcagattgagtcgttaaggaaagcttttcctgaagaaaaaatatatttatctctgccggaagagtatcacattggtaggtatgatcaacaatactttaatgaaaatgatattacaatactaaaaacttcttatactttatcacaaggacatagtctgtatcggttgttaaatgattttaacttaatcgaatgtgataaactaattattaattatggtgatactttttatcatacctttgaagctacgagtgatgattatttttatgtatctaataacatcggctactataaccgagcatctgttgtaattaatggtgataagcttattataaataaagataatccagctgttgataatggtgagttggttatttctggtttcttttgtttcagtaatattgggaggttatgcgaatgcttgaaaaagatgaactatgattttctgtcgtcttctgaattgtattactcgtcggttgcaacaaaagttttaaaaaatgatcattggtatgattttggtcatttaaacagcttttttacatcaagaacgagtatcactactgagcgtgagtttaatagtcttcaaataaataatttttgtgtgataaaaaaaagtaagaagaaagcaaaaatgctaggcgaagcgaattggtttttgagtttgccgcctgcattgagtatcttcactccgagggtttttgatgttagagaagataatgattatgcagagtatacaatagaatatctttataatcttcctttgtctgatatggcagtgttttgtgagttacctaagtcatgttgggtgaatgtgtttaactcgtgtcgagatttcttaaatagatcaaaagaatactctaatagtaaaaaaacgctagatatgtctgaactggcatcatatgataaattgtactatgataagaccctaaaaaggttacatgattttgaattgacaacaaacatacaattgactaaagatgttatatttaatggtaaaagatttccatcgataaaaaaatagcagaaatatcatcacgctttattaacccaactaagagtgatgatgttataattacacatggcgatttttgctttagtaatatattatacgattttagaagtcagagaattaaattaatcgatcccagaggtattaactataacaatgagttaagcatatggggagatataaggtatgacttggctaagttatgtcactcggtaattggatgttatgatcttattatcgctcagaaatttttgctggaatacgattatgtagataacagaattgaatttgaactatatgatgataactacagcgacatagtgaattcatttttaggcgtgtttaattcttttgaagaatttgggtatactattgaagagatacaatgtataaccatacatttatttttatcaatgttgccgcttcattctgattctaaacttagacaagaagcatttatagcaaatgcttatagactgttcagtttgttaaatttggagtatttatgacaacgattgtaattcctatggcaggacaatcatcacgcttttataaagcaggttatgatgttcctaagtataagttaaaaattaataacaaaagcgtattttttcattcactacgaagttttctaaaatttaaggaccatttttttctaattataggtattaaaggcatactagatgaaaagtttgttcaatatgaaatggccgaattaggtataaatgattttgaaattgttttgctcgaacagcaaacaaatggtcaagcagagacggttgtgaaaggagtaaaaaattgcaagagagatattagtgacattttaatttttaatatagatacttttcgcagagagattaatttgcctgataattttagtttgaaagattgctctgggtatttggaaacatttttagggtcaggtgagaattggagtaatattatgcctgaatcagatgtccctaatcaagtaaaaatgactgcggaaaaacaaaatatatctgaatattgttgcactggtttatattattttggtgattataagttgttgaatatagcttataataactggctagaaaatgaagatactactaaagaggtgtatatcgctcctctatataatcatttgattacatgtgggcacaaaattttttataccgttataaacagatgtgatgtggtgttctgtggtgtgcctaatgaatatgaaaaaataaagaatgggtttgactggaaatgagacagcacagaaaattagtcagttatgctgtgattattacttttttgtttatacctatacaagttattctgtcgttaatttttaactattatttagctaaaaacgttgctctggatgttttaaatgcttggtatatgttttttgcactcatttcccttttttctttgttggaattttcctatcccatattagcaattaatttttttaacagctatcagggaaattgtaaaggagctctgtctttcttcataaagaaatcattaggggtagtaataccccttcagatcatatttgttgtaatttatagtttatttgttaacccgctttttatttattttggtatgggtttggtggtacgtagtatatcgaatattattaatgcatgtgtatatgcaaaacaaaatattattatcgataaagtttatcgatttctgtacgccataattatgccaactgctttccttttcttgttttatcattctgataaaaacattgatttgtatgatttaactcttgtatggtttttgagctcactattctgcttaatatattctttgtcatttttactccttaagcataagaagtctgtcaacaggagcgaaaaatcattccatctaccattcaagtataaccttaagcttttttttactgtttttcctgcaattttcatctatacattgtctatatattatttgaaaatatttggttcagaaagcagttctagttctactattatatatggtttctttttgcaaatatttaatgtttataatttgataattattttggttgcaagttatttgatgcctacgctctcaaagcgtttccatgatggtgttgatgttatccctctcacacttaaactattagatatcagtgtacttatatctgttatttctacttcttttgtggcattgttaggtattcctgttgcggagtatattcttaaggggaaaatagatgtaatatcctactattatatagtagcaataattattatattctatattgaaacatgtcaggttattctaacctcaattggcactgccattggtatttacgattttcataggcaaagtattatatcagcagttatggttttaattctgtcatgttttttaatacctcattacaatgctgagggtttgatgtatagtatcattatatctcaattctttacatgctttatttataaccctaaacgtgttataaataaaattggattagataaagttaattatattaaaagattaatggtgcactttttgtttatgttgactcttatttttatggggttatattttcaaaatgatgatatattgccgagagttgtgattctttcttttatcacattgattggtgtttgttttctctacccaaatgtgaaaaaaataattgttgatttcttgtagttttttaattttatttgcattttaaatggaaaatcttgttctatcttagaaaggagttaacaatgctgaagcaacagatcggcaaat

SEQ ID NO: 5 - DNA, 1215 bp, Salmonella enterica
  source 1..1215: mol_type = genomic DNA
atgctaaccacatcattaacgttaaataaagagaaatggaagccgatctggaataaagcgctggtttttctttttgttgccacgtattttctggatggtattacgcgttataaacatttgataatcatacttatggttatcaccgcgatttatcaggtctcacgctcaccgaaaagtttcccccctcttttcaaaaatagcgtattttatagcgtagcagtattatcattaatccttgtttattccatactcatatcgccagatatgaaagaaagtttcaaggaatttgaaaatacggtactggagggcttcttattatatactttattaattcccgtactattaaaagatgaaacaaaagaaacggttgcgaaaatagtacttttctcctttttaacaagtttaggacttcgctgccttgcagagagtattctgtatatcgaggactataataaagggattatgccattcataagctatgcgcatcgacatatgtccgattccatggttttcttatttccagcattattgaatatttggctgtttagaaaaaatgcaattaagttggtttttttggtgcttagcgccatctaccttttctttatcctgggaaccctatcgcgaggggcatggttggcggtgcttatagtaggtgttctgtgggcaatactgaaccgccaatggaagttaataggagttggtgccattttattagccattatcggcgctttggttatcactcaacataataacaaaccagacccagaacatttactgtataaattacagcagacagatagctcatatcgttatactaacggaacccagggcaccgcgtggatactgattcaggaaaacccgatcaagggctacggctatggtaatgatgtgtatgatggtgtttataataaacgcgttgtcgattatccaacgtggacctttaaagaatctatcggtccgcataataccattctgtacatctggtttagtgcaggcatattgggtctggcgagcctggtctatttatatggcgctatcatcagggaaacagccagctctaccctcaggaaagtagagataagcccctacaatgctcatctcttgctatttttatctttcgtcggtttttatatcgttcgtggcaattttgaacaggtcgatattgctcaaattggtatcattaccggttttctgctggcgctaagaaatagataa

SEQ ID NO: 6 - protein, 404 aa, Salmonella enterica
MLTTSLTLNKEKWKPIWNKALVFLFVATYFLDGITRYKHLIIILMVITAIYQVSRSPKSFPPLFKNSVFYSVAVLSLILVYSILISPDMKESFKEFENTVLEGFLLYTLLIPVLLKDETKETVAKIVLFSFLTSLGLRCLAESILYIEDYNKGIMPFISYAHRHMSDSMVFLFPALLNIWLFRKNAIKLVFLVLSAIYLFFILGTLSRGAWLAVLIVGVLWAILNRQWKLIGVGAILLAIIGALVITQHNNKPDPEHLLYKLQQTDSSYRYTNGTQGTAWILIQENPIKGYGYGNDVYDGVYNKRVVDYPTWTFKESIGPHNTILYIWFSAGILGLASLVYLYGAIIRETASSTLRKVEISPYNAHLLLFLSFVGFYIVRGNFEQVDIAQIGIITGFLLALRNR

SEQ ID NO: 7 - DNA, 1011 bp, Salmonella enterica
  source 1..1011: mol_type = genomic DNA
atgccgattactatagggaatggttttttaaaaagtgaaatccttaccaactccccaaggaatacgaaagaagcatggtggaaagttttatgggaaaaaattaaagacttctttttttctactggcaaagcaaaagcggaccgttgtctacatgagatgttgtttgccgaacgcgcccccacacgagagcggcttacagagattttttttgagttgaaagagttagcctgcgcatcgcaaagagatagatttcaggttcataatcctcatgaaaatgatgccaccattattcttcgcatcatggatcaaaacgaagagaacgaattgttacgtatcactcaaaataccgatacctttagctgtgaagtcatggggaatctttattttttaatgaaagatcgcccggatattttaaaatcgcatccacaaatgacggccatgattaagagaagatatagcgaaatcgtagactaccccctcccttcgacattatgtctcaatcctgctggcgcgccgatattatcggttccattagacaacatagaggggtatttatatactgaattgagaaaaggacatttagatgggtggaaagcgcaagaaaaggcaacctacctggcagcgaaaattcagtctgggattgaaaagacaacgcgcattttacaccatgcgaatatatccgaaagtactcagcaaaacgcatttttagaaacaatggcgatgtgtggattaaaacagcttgaaataccaccaccgcatacccacatacctattgaaaaaatggtaaaagaggttttactagcggataagacgtttcaggcgttcctcgtaacggatcccagcaccagccaaagtatgttagctgagatagtcgaagccatctctgatcaggtttttcacgccatttttagaatagacccccaggctatacaaaaaatggcggaagaacagttaaccacgctacacgttcgctcagaacaacaaagcggctgtttatgttgttttttataa

SEQ ID NO: 8 - protein, 336 aa, Salmonella enterica
MPITIGNGFLKSEILTNSPRNTKEAWWKVLWEKIKDFFFSTGKAKADRCLHEMLFAERAPTRERLTEIFFELKELACASQRDRFQVHNPHENDATIILRIMDQNEENELLRITQNTDTFSCEVMGNLYFLMKDRPDILKSHPQMTAMIKRRYSEIVDYPLPSTLCLNPAGAPILSVPLDNIEGYLYTELRKGHLDGWKAQEKATYLAAKIQSGIEKTTRILHHANISESTQQNAFLETMAMCGLKQLEIPPPHTHIPIEKMVKEVLLADKTFQAFLVTDPSTSQSMLAEIVEAISDQVFHAIFRIDPQAIQKMAEEQLTTLHVRSEQQSGCLCCFL

SEQ ID NO: 9 - DNA, 1083 bp, Escherichia coli
  source 1..1083: mol_type = genomic DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 10 - protein, 360 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 11 - DNA, 1176 bp, Salmonella enterica
  source 1..1176: mol_type = genomic DNA
atgcaaaaactcattaactcagtgcaaaactatgcctggggaagtaaaactgcgttaacggaactttatggcatcgccaatccgcagcagcagccaatggctgaactctggatgggcgcgcatcccaaaagcagctcgcgaatcaccaccgccaacggcgaaaccgtctccctgcgtgacgccatcgaaaagaataaaaccgccatgctgggcgaagcggtagccaaccgtttcggcgaactgccgtttctgtttaaagtactgtgcgccgcacaaccgctctctattcaggtgcacccgaataaacgcaactccgaaatcggtttcgcgaaagaaaatgcggcgggtatccccatggatgccgcagagcggaactataaagatcctaaccataaaccagagctggtttttgccctgacgcctttcctggcgatgaacgcgttccgcgaattttctgacattgtctctttactgcaacctgtcgccggcgcgcattccgctatcgcccactttttgcaggtgccgaatgctgaacgtctgagccagcttttcgccagcctgttgaatatgcaaggcgaagaaaaatcccgcgcgttagccgtactcaaagcggcgcttaacagccagcaaggcgaaccgtggcaaacgatccgcgtgatttcagagtattatcctgacgacagcgggcttttctctcctttgttgctgaatgtggtcaaactgaatcccggcgaggcgatgttcctgtttgctgaaacgcctcatgcttatctgcagggcgttgcgctggaagtcatggcgaactccgataacgttctgcgcgctggccttacgccaaaatatatcgacatccctgagctggtcgcgaacgtgaagttcgaacctaagcctgccggcgagttgctgactgccccggtgaaaagcggcgcggagctggacttcccaattccggttgacgattttgctttttcactgcacgacctggcgcttcaggagacgagcatcggccaacacagcgccgcgattctgttctgcgttgagggtgaggcggtgttacgtaaagatgaacagcgtctggtactgaagccgggtgaatctgcctttatcggcgcggatgagtctccggttaacgccagcggcacgggccgtttagcgcgtgtttataacaagctgtag

SEQ ID NO: 12 - DNA, 1104 bp, Salmonella enterica
  source 1..1104: mol_type = genomic DNA
atgaaaaatgttggttttatcggctggcgcggagtggtcggctctgttctcatgcaacgcatggtagaggagcgcgatttcgacgctattcgccctgttttcttttctacctcccagtttggacaggcggcgcccaccttcggcgacacctccggcacgctacaggacgcttttgacctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcctag

SEQ ID NO: 13 - DNA, 56 bp, synthetic construct
  source 1..56: mol_type = other DNA
cgaattcctgcagcccgggggatccactagtcgtcgacaagccaatgattcagtac

SEQ ID NO: 14 - DNA, 56 bp, synthetic construct
  source 1..56: mol_type = other DNA
accgcggtggcggccgctctagaactagtgacatccaccaggtagtttccgtcttc

SEQ ID NO: 15 - DNA, 51 bp, synthetic construct
  source 1..51: mol_type = other DNA
accgcggtggcggccgctctagaactagtgctgataaagctcaggattctg

SEQ ID NO: 16 - DNA, 53 bp, synthetic construct
  source 1..53: mol_type = other DNA
cgaattcctgcagcccgggggatccactagtcagcttaacggtcttcaggttc

SEQ ID NO: 17 - DNA, 54 bp, synthetic construct
  source 1..54: mol_type = other DNA
cgaattcctgcagcccgggggatccactagtgaaaacagaacgtcgtcgggatc

SEQ ID NO: 18 - DNA, 27 bp, synthetic construct
  source 1..27: mol_type = other DNA
taacacggaacaggtgcaaaatcgctg

SEQ ID NO: 19 - DNA, 52 bp, synthetic construct
  source 1..52: mol_type = other DNA
cagcgattttgcacctgttccgtgttagtctctctgtattccttccctactc

SEQ ID NO: 20 - DNA, 54 bp, synthetic construct
  source 1..54: mol_type = other DNA
accgcggtggcggccgctctagaactagttgtttgggctatgaccgatggctac

SEQ ID NO: 21 - DNA, 53 bp, synthetic construct
  source 1..53: mol_type = other DNA
cgaattcctgcagcccgggggatccactagtctgtttgcggtatgcggtgaac

SEQ ID NO: 22 - DNA, 25 bp, synthetic construct
  source 1..25: mol_type = other DNA
gttcacttccagagcgggctgttac

SEQ ID NO: 23 - DNA, 46 bp, synthetic construct
  source 1..46: mol_type = other DNA
gtaacagcccgctctggaagtgaacttcattgcttacggccttgag

SEQ ID NO: 24 - DNA, 52 bp, synthetic construct
  source 1..52: mol_type = other DNA
accgcggtggcggccgctctagaactagtttcgatgccagatgggcatgtac

SEQ ID NO: 25 - DNA, 52 bp, synthetic construct
  source 1..52: mol_type = other DNA
cgaattcctgcagcccgggggatccactagtagtaccagaagcaacagcatc

SEQ ID NO: 26 - DNA, 26 bp, synthetic construct
  source 1..26: mol_type = other DNA
ctggtatgatgctggcgctgattctg

SEQ ID NO: 27 - DNA, 61 bp, synthetic construct
  source 1..61: mol_type = other DNA
cagaatcagcgccagcatcataccagatttaaatcaaagcggtgtcggcgcgttgttgtag

SEQ ID NO: 28 - DNA, 50 bp, synthetic construct
  source 1..50: mol_type = other DNA
accgcggtggcggccgctctagaactagtaccggtatctgacaatgtctg

SEQ ID NO: 29 - DNA, 56 bp, synthetic construct
  source 1..56: mol_type = other DNA
cgaattcctgcagcccgggggatccactagtctcaggagttaagataaggacggtg

SEQ ID NO: 30 - DNA, 53 bp, synthetic construct
  source 1..53: mol_type = other DNA
accgcggtggcggccgctctagaactagtgggagaggattaatcatgattgac

SEQ ID NO: 31 - DNA, 46 bp, synthetic construct
  source 1..46: mol_type = other DNA
atcagcgccagcatcataccagatttgccatcgtccacatatccac

SEQ ID NO: 32 - DNA, 47 bp, synthetic construct
  source 1..47: mol_type = other DNA
acaacgcgccgacaccgctttgatttgcttgagtactaggccaactg

SEQ ID NO: 33 - DNA, 38 bp, synthetic construct
  source 1..38: mol_type = other DNA
atttaaatggtgcgtattaagcttaccgagaagtactg

SEQ ID NO: 34 - DNA, 47 bp, synthetic construct
  source 1..47: mol_type = other DNA
taagcttaatacgcaccatttaaatcaccaaccatttatcacggttg

SEQ ID NO: 35 - DNA, 47 bp, synthetic construct
  source 1..47: mol_type = other DNA
cggtaagcttaatacgcaccatttgccgatctgttgcttcagcattg

SEQ ID NO: 36 - DNA, 53 bp, synthetic construct
  source 1..53: mol_type = other DNA
caaccgtgataaatggttggtgattttgaaagcttgatcggatatgacggctg

(19) United States
(12) Patent Application Publication
(10) Pub. No.: US 2025/0295747 A1
(43) Pub. Date: Sep. 25, 2025

(54) RECOMBINANT ATTENUATED SALMONELLA VACCINES (RASVS) AGAINST INFECTIONS BY AVIAN PATHOGENIC ESCHERICHIA COLI (APEC)

(71) Applicant: Huvepharma Inc., Peachtree City, GA (US)

(72) Inventors: Chang-Ho Baek, Newberry, FL (US); Ryan Vander Veen, Peachtree City, GA (US)

(73) Assignee: Huvepharma Inc., Peachtree City, GA (US)

(21) Appl. No.: 19/082,476

(22) Filed: Mar. 18, 2025

Related U.S. Application Data
(60) Provisional application No. 63/568,108, filed on Mar. 21, 2024.

(51) Int. Cl. A61K 39/108; A61K 39/00; C07K 14/245; C12N 15/52

(52) U.S. Cl. CPC A61K 39/0258; C07K 14/245; C12N 15/52; A61K 2039/522; A61K 2039/523; A61K 2039/552

(57) ABSTRACT

The present disclosure provides compositions and methods for making and using Salmonella recombinant bacteria that express LPS O78 antigen and/or virulence factors of avian pathogenic Escherichia coli (APEC) as vaccines to prevent colibacillosis infections.

### REFERENCE TO RELATED APPLICATION

This application claims priority to U.S. Provisional Patent Application No. 63/568,108, filed on Mar. 21, 2024, the contents of which are incorporated herein by reference in its entirety.

### REFERENCE TO SEQUENCE LISTING

The instant application contains a Sequence Listing which has been submitted electronically in XML format and is hereby incorporated by reference in its entirety. Said XML copy, created on Mar. 17, 2025, is named 139465-00102-SeqList.XML and is 279,169 bytes in size.

### BACKGROUND

Avian pathogenic Escherichia coli (APEC) is a type of extraintestinal pathogenic microorganism that causes diverse systemic infections in poultry, including chickens, turkeys, ducks, and many other avian species. The most common diseases caused by APEC in chickens includes airsacculitis, cellulitis, coligranuloma, egg peritonitis, omphalitis, osteomyelitis/arthritis, perihepatitis, pericarditis, and salphingitis (collectively known as colibacillosis infections).

Colibacillosis caused by APEC is a devastating disease of poultry that results in severe economic losses annually to the poultry industry worldwide. Multiple APEC serotypes have been associated with colibacillosis cases in the field outbreaks; however, three serotypes (O78, O2, and O1) account for the majority (more than 80%) of the cases. The pathogenicity of APEC is still poorly understood. Thus, there is a need for improved vaccines against APEC infection.

### SUMMARY

Construction of novel recombinant attenuated Salmonella vaccines (RASVs) protecting avians against systemic infections by avian pathogenic Escherichia coli (APEC) is disclosed herein. In particular, disclosed herein is a method for the inter-genus serotype conversion by replacing the Salmonella O4-antigen gene cluster (rfb_{O4}) in RASV strains with the APEC O78-antigen gene cluster (rfb_{O78}), providing serotype converted RASV strains which successfully produce APEC O78-antigen but no longer produce S. typhimurium O4-antigen. Additionally, the phosphomannose isomerase (pmi) mutant will have a defect to produce LPS O-antigen in a mannose limitation environment like in vivo since the phosphomannose is a key building block for the LPS O-antigen production.

In one aspect, the disclosure provides a recombinant Salmonella bacterium, wherein the bacterium expresses lipopolysaccharide (LPS) O78 antigen of avian pathogenic Escherichia coli (APEC) and does not express Salmonella LPS O4 antigen.

In some embodiments, the recombinant Salmonella bacterium comprises APEC's rfb_{O78} gene cluster. In some embodiments, the recombinant Salmonella bacterium further comprises a deletion mutation in the Salmonella rfb_{O4} gene cluster. In some embodiments, the APEC's rfb_{O78} gene cluster replaces the entire Salmonella rfb_{O4} gene cluster in the genome of the Salmonella bacterium.

In some embodiments, the rfb_{O4} gene cluster comprises a nucleic acid sequence having at least 85%, at least 90%, or at least 95% sequence identity to the sequence of SEQ ID NO: 4 or wherein the rfb_{O4} gene cluster comprises the nucleic acid sequence of SEQ ID NO: 4.

In some embodiments, the Salmonella recombinant bacterium further expresses one or more APEC virulence factors. In some embodiments, the Salmonella recombinant bacterium comprises one or more nucleotides encoding one or more APEC virulence factors. In some embodiments, the Salmonella recombinant bacterium comprises one or more APEC plasmids that comprises nucleotides encoding one or more APEC virulence factors.

In some embodiments, the APEC plasmid is plasmid pAPEC-1, pAPEC-2, pAPEC-3, or a combination thereof, derived from APEC strain X⁷¹²². In some embodiments, the recombinant Salmonella bacterium comprises all three plasmids pAPEC-1, pAPEC-2 and pAPEC-3.

In some embodiments, the pAPEC-1 plasmid comprises a nucleic acid sequence having at least 85%, at least 90%, or at least 95% sequence identity to the sequence of SEQ ID NO: 1. In some embodiments, the pAPEC-2 plasmid comprises a nucleic acid sequence having at least 85%, at least 90%, or at least 95% sequence identity to the sequence of SEQ ID NO: 2. In some embodiments, the pAPEC-3 plasmid comprises a nucleic acid sequence having at least 85%, at least 90%, or at least 95% sequence identity to the sequence of SEQ ID NO: 3

In some embodiments, the recombinant Salmonella bacterium further comprises a deletion mutation in the lrp gene (Δlrp).

In some embodiments, the recombinant Salmonella bacterium comprises an asd gene (asd+). In some embodiments, the asd gene is inserted into the pAPEC-1 plasmid. In some embodiments, the asd gene comprises a nucleic acid sequence having at least 85%, at least 90%, or at least 95% sequence identity to the sequence of SEQ ID NO: 12, or the asd gene comprises the nucleic acid sequence of SEQ ID NO: 12.

In some embodiments, the recombinant Salmonella bacterium comprises a pmigene (pmi+). In some embodiments, the pmigene comprises a nucleic acid sequence having at least 85%, at least 90%, or at least 95% sequence identity to the sequence of SEQ ID NO: 11 or the pmigene comprises the nucleic acid sequence of SEQ ID NO: 11.

In some embodiments, the recombinant Salmonella bacterium further comprises, or is derived from a parent Salmonella bacterium strain comprising: a araC P_{araBAD}-regulated murA gene (ΔPₘᵤᵣₐ::TT araC P_{araBAD} murA deletion-insertion mutation); an insertion of a c2 gene operably linked to an arabinose regulatable promoter araC P_{araBAD} (araC P_{araBAD} c2), optionally wherein the araC P_{araBAD} c2 gene cassette is inserted into and thereby inactivate the endogenous asd gene (ΔasdA::TT araC P_{araBAD} c2 deletion-insertion mutation); a deletion mutation in the gmd and a deletion in the fcl genes (Δ(gmd-fcl)); and a deletion-insertion mutation that inactivates the expression of a relA gene and inserts a lacl gene (ΔrelA::araC P_{araBAD} lacl TT deletion-insertion mutation).

In some embodiments, the recombinant Salmonella bacterium further comprises an araC P_{araBAD}-regulated murA gene (ΔP_{murA}::TT araC P_{araBAD} murA deletion-insertion mutation); an insertion of a c2 gene operably linked to an arabinose regulatable promoter araC P_{araBAD} (araC P_{araBAD} c2), optionally wherein the araC P_{araBAD} c2 gene cassette is inserted into and thereby inactivate the endogenous asd gene (ΔasdA::TT araC P_{araBAD} c2 deletion-insertion mutation); a deletion mutation of the gene cluster wza-wcaM (Δ(wza-wcaM)); a deletion-insertion mutation that inactivates the expression of a relA gene and inserts a lacl gene (ΔrelA::araC P_{araBAD} lacl TT deletion-insertion mutation) a deletion mutation in the recF gene (ΔrecF); a deletion mutation in the waaL gene (ΔwaaL46); and a deletion in a pagL gene and an insertion of a rhaRS P_{rhaBAD}-regulated waaL gene (ΔpagL::TT rhaRS P_{rhaBAD} waaL).

In some embodiments, the recombinant Salmonella bacterium is a Salmonella enterica bacterium. In some embodiments, the bacterium is a Salmonella enterica subsp. enterica serovar Paratyphi A bacterium, a Salmonella enterica subsp. enterica serovar Enteritidis bacterium, a Salmonella enterica subsp. enterica serovar Typhi bacterium, a Salmonella enterica subsp. enterica serovar Typhimurium bacterium, Salmonella enterica subsp. enterica serovar Dublin, Salmonella Pullorum, Salmonella Gallinarum, or Salmonella enterica subsp. enterica serovar Choleraesuis.

In one aspect, the disclosure provides pharmaceutical composition comprising the recombinant Salmonella bacterium described herein, and a pharmaceutically acceptable carrier.

In a certain aspect, the disclosure provides a vaccine comprising the recombinant Salmonella bacterium or the pharmaceutical composition described herein.

In another aspect, the disclosure provides a method for eliciting an immune response against APEC in a subject, the method comprising administering to the subject an effective amount of the pharmaceutical composition or the vaccine described herein.

In another aspect, the disclosure provides a method for vaccinating a subject against APEC, the method comprising administering to the subject an effective amount of the pharmaceutical composition or the vaccine described herein.

In some embodiments, the subject is a chicken, turkey, goose, or duck.

In some embodiments, the pharmaceutical composition or vaccine is administered by spray or oral immunization.

In another aspect, the disclosure provides a method for producing the recombinant Salmonella bacterium, the method comprising culturing the bacterium in media.

### BRIEF DESCRIPTION OF THE DRAWINGS

FIG. 1 shows a scheme to develop RASVs against APEC infections.

FIG. 2 shows schematics of APEC virulence plasmids in the wild-type APEC strain X⁷¹²².

FIG. 3 shows schematics of LPS and colonic acid coding areas in S. typhimurium and APEC.

FIG. 4 shows a schematic of pCHSUI-APEC-O78 (21,542 bp) map.

FIG. 5 shows lineage of the RASV backbone strain X¹²³⁴¹.

FIG. 6 shows construction of the RASV-based APEC vaccines.

FIG. 7 shows siderophore production in the RASV-based APEC vaccines.

FIG. 8 shows LPS O-antigen production in RASV-based APEC vaccines.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### Definitions

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

As used herein, the term “derived from” or “derivative of” refers to a composition (e.g., a gene, a plasmid, a bacterium) that results from modifications or manipulation of an original composition. For example, insertion of a selective marker gene into an original pAPEC-1 plasmid results in a plasmid derived from pAPEC-1, or a pAPEC-1 derivative plasmid

As used herein, the term “gene” refers to a nucleic acid fragment that encodes a protein or a fragment thereof, or a functional or structural RNA molecule, and may optionally include a regulatory sequence preceding (5′ non-coding sequences) and following (3′ non-coding sequences) the coding sequence of the nucleic acid. In some embodiments, a “gene” does not include regulatory sequences preceding and following the coding sequence.

In one embodiment, the gene is a heterologous gene. In another embodiment, the nucleic acid is a heterologous nucleic acid. As used herein, the terms “heterologous gene” or “heterologous nucleic acid” refer to a gene or a nucleic acid sequence present in a recombinant cell, e.g., bacterium, that is not normally found in the wild-type cell, e.g., bacterium, in nature. In some embodiments, the heterologous gene or heterologous nucleic acid is exogenously introduced into a given cell. In some embodiments, a heterologous gene may include a gene, or fragment thereof, introduced into a non-native host cell. In some embodiments, the term “heterologous gene” includes a second copy of a native gene, or fragment thereof, that has been introduced into the host cell in addition to the corresponding native gene. A heterologous nucleic acid may also include, in some embodiments, a gene sequence that is naturally-found in a given cell but which has been modified, e.g., by regulation by a different promoter sequence, to expresses an unnatural amount of the nucleic acid and/or the polypeptide which it encodes; and/or two or more nucleic acid sequences that are not found in the same relationship to each other in nature.

As used herein, the term “endogenous gene” refers to a native gene that is present in its natural location in the genome of an organism (e.g., a bacterial chromosome).

A “promoter” as used herein, refers to a nucleic acid sequence that is capable of controlling the expression of a coding sequence or gene. A promoter may comprise one or more specific transcriptional regulatory sequences to further enhance expression and/or to alter the spatial expression and/or temporal expression of a nucleic acid. For example, a promoter may include one or more nucleic acids that are specifically recognized by a transcriptional activator protein (e.g., an enhancer element), a transcriptional repressor protein, a polymerase, and the like. The term “operably linked,” as used herein, means that expression of a nucleic acid sequence is under the control of a promoter with which it is spatially connected. A promoter may be positioned 5′ (upstream) of the nucleic acid sequence under its control. The distance between the promoter and a nucleic acid sequence to be expressed may be approximately the same as the distance between that promoter and the native nucleic acid sequence it controls. As is known in the art, variation in this distance may be accommodated without loss of promoter function. The nucleic acid sequences of the promoters described herein are known in the art, and methods of operably-linking these promoters to a gene (e.g., a gene encoding a repressor) are known in the art.

In some embodiments, the promoter for use as described herein may be regulated directly or indirectly by a sugar. For example, in some embodiments, the promoter is responsive to the level of arabinose, otherwise referred to herein as an “arabinose-regulatable promoter”. Generally speaking, arabinose may be present during the in vitro growth of a bacterium, while typically absent from host tissue. In one embodiment, the promoter is derived from an araC-P_{araBAD} system from Escherichia coli. The araC P_{araBAD} system is a tightly regulated expression system, which has been shown to work as a strong promoter induced by the addition of low levels of arabinose. The araC-araBAD promoter is a bidirectional promoter controlling expression of the araBAD nucleic acid sequences in one direction, and the araC nucleic acid sequence in the other direction.

For convenience, the portion of the araC-araBAD promoter that mediates expression of the araBAD nucleic acid sequences, and which is controlled by the araC nucleic acid sequence product, is referred to herein as P_{araBAD}. For use as described herein, a cassette with the araC nucleic acid sequence and the araC-araBAD promoter may be used. This cassette is referred to herein as araC P_{araBAD}. The AraC protein is both a positive and negative regulator of P_{araBAD}. In the presence of arabinose, the AraC protein is a positive regulatory element that allows expression from P_{araBAD}. In the absence of arabinose, the AraC protein represses expression from P_{araBAD}. Other enteric bacteria contain arabinose regulatory systems homologous to the araC-araBAD system from E. coli, including, for example, S. typhimurium. For example, the E. coli AraC protein only activates E. coli P_{araBAD} (in the presence of arabinose) and not S. typhimurium P_{araBAD}. Thus, an arabinose regulated promoter may be used in a recombinant bacterium that possesses a similar arabinose operon, without substantial interference between the two, if the promoter and the operon are derived from two different species of bacteria. Generally speaking, the concentration of arabinose necessary to induce expression is typically less than about 2% (w/w) in a culture media. In some embodiments, the concentration is less than about 1.5%, 1%, 0.5%, 0.2%, 0.1%, or 0.05% (w/w) in a culture media. In other embodiments, the concentration is 0.05% or below, e.g. about 0.04%, 0.03%, 0.02%, or 0.01% (w/w). In an exemplary embodiment, the concentration is about 0.05% (w/w) in a culture media.

In still other embodiments, the promoter used herein is responsive to the level of rhamnose in the environment, otherwise referred to herein as a “rhamnose-regulatable promoter”. Analogous to the araC-P_{araBAD} system described above, the rhaRS-P_{rhaBAD} activator-promoter system is tightly regulated by rhamnose. Expression from the rhamnose promoter (Pᵣₕₐ) is induced to high levels in the presence of rhamnose. In some embodiments, the bacteria are cultured in the presence of rhamnose. Rhamnose is commonly found in bacteria but rarely found in human subjects. The rhaBAD operon is controlled by the P_{rhaBAD} promoter. This promoter is regulated by two activators, RhaS and RhaR, and the corresponding nucleic acid sequences belong to one transcription unit that is located in the opposite direction of the rhaBAD nucleic acid sequences. In the presence of L-rhamnose, RhaR binds to the P_{rhaRS} promoter and activates the production of RhaR and RhaS. RhaS together with L-rhamnose, in turn, bind to the P_{rhaBAD} and the P_{rhaT} promoters and activates the transcription of the structural nucleic acid sequences. Full induction of the arabinose, maltose and rhamonse regulated promoters described herein requires binding of the Crp-cAMP complex, which is a key regulator of catabolite repression.

Although both L-arabinose and L-rhamnose act directly as inducers of the expression of regulons that mediate their catabolism, important differences exist in regard to the regulatory mechanisms. L-Arabinose acts as an inducer with the activator AraC in the positive control of the arabinose regulon. However, the L-rhamnose regulon is subject to a regulatory cascade, and is therefore subject to even tighter control than the araC-P_{araBAD} system. L-Rhamnose acts as an inducer with the activator RhaR for synthesis of RhaS, which in turn acts as an activator in the positive control of the rhamnose regulon. In the present disclosure, rhamnose may be used to interact with the RhaR protein and then the RhaS protein may activate transcription of a nucleic acid sequence operably-linked to the P_{rhaBAD} promoter.

As used herein, the term “exogenous” refers to a substance (e.g., a nucleic acid or polypeptide) present in a cell other than its native source. The term exogenous can refer to a nucleic acid or a protein that has been introduced by a process involving the hand of man into a biological system such as a cell or organism in which it is not normally found or in which it is found in undetectable amounts. A substance can be considered exogenous if it is introduced into a cell or an ancestor of the cell that inherits the substance. In contrast, the term “endogenous” refers to a substance that is native to the biological system or cell.

As used herein, the term “host cell” refers to a cell in an organism to which the recombinant bacterium is being administered in order to, for example, induce an immune response. In one embodiment, a host is a bird, equine, or human and a host cell refers, respectively, to a bird cell, an equine cell, or a human cell. “Host” and “subject” are used interchangeably in the present disclosure.

A “nucleic acid” or “nucleic acid sequence” may be any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA, rRNA, and tRNA.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (35); Bauer et al. (36); Craik (37); Smith et al. (38); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

A “pharmaceutical composition,” as used herein, refers to a composition comprising an active ingredient (e.g., a recombinant bacterium described herein) with other components such as a physiologically suitable carrier and/or excipient.

As used herein, the term “pharmaceutically acceptable carrier” or a “pharmaceutically acceptable excipient” refers to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline (e.g., phosphate-buffered saline (PBS)); (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

A “plasmid” or “vector” includes a nucleic acid construct designed for delivery to a host cell or transfer between different host cells. The nucleic acid incorporated into the plasmid can be operatively linked to an expression control sequence when the expression control sequence controls and regulates the transcription and translation of that polynucleotide sequence.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. The terms “protein” and “polypeptide” as used herein refer to both large polypeptides and small peptides. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “recombinant bacterium” refers to a bacterial cell that has been genetically modified from its native state. For instance, a recombinant bacterium may comprise one or more nucleotide insertions, nucleotide deletions, nucleotide rearrangements, and nucleotide modifications. These genetic modifications may be introduced into the chromosome of the bacterium, or alternatively be present on an extrachromosomal nucleic acid (e.g., a plasmid). Recombinant bacteria of the disclosure may comprise a nucleic acid located on a plasmid. Alternatively, the recombinant bacteria may comprise a nucleic acid located in the bacterial chromosome (e.g., stably incorporated therein). In some embodiments, the recombinant bacterium is avirulent. In some embodiments the recombinant bacterium exhibits reduced virulence. In some embodiments, the recombinant bacterium is non-virulent. In some embodiments, the recombinant bacterium is pathogenic. In some embodiments, the recombinant bacterium is attenuated. In another embodiment, the recombinant bacterium is a recombinant derivative of a pathogenic bacterium.

The term “statistically significant” or “significantly” refers to statistical significance and generally means a two standard deviation (2SD) or greater difference.

As used herein, the term “virulence factor” refers to a molecule that assist a pathogenic bacterium in colonizing the host at the cellular level. Virulence factors can be either secretory, membrane associated or cytosolic in nature. Typically, the cytosolic factors facilitate the bacterium to undergo adaptation such as metabolic, physiological and morphological shifts. The membrane associated virulence factors usually aid the bacterium in adhesion and evasion of the host cell. The secretory factors are important components of bacterial armoury which help the bacterium wade through the innate and adaptive immune response mounted within the host. In extracellular pathogens, the secretory virulence factors can act synergistically to kill the host cells.

Other than in the operating examples, or where otherwise indicated, all numbers expressing quantities of ingredients or reaction conditions used herein should be understood as modified in all instances by the term “about.” The term “about” when used in connection with percentages can mean±1%.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Compositions

The present disclosure provides, in one aspect, a Salmonella recombinant bacterium capable of expressing lipopolysaccharide (LPS) O78 antigen of avian pathogenic Escherichia coli (APEC) without expressing the native Salmonella LPS O4 antigen. The invention is based on the surprising finding that conversion of Salmonella LPS O4 serotype to APEC LPS O78 serotype results in strong O78 antigen production by the recombinant Salmonella bacterium.

In another aspect, the present disclosure further provides a recombinant Salmonella bacterium capable of expressing APEC's virulence factors, which is effective in inducing specific antibody response against APEC pathogens.

In some embodiments, the recombinant bacterium described herein is modified such that the expression of one or more genes, e.g., virulence genes, can be regulated in a sugar-responsive manner. In some embodiments, one or more endogenous genes, e.g., virulence genes, are deleted from the bacterial chromosome. In some embodiments, the deletion is a partial deletion of the endogenous gene. In some embodiments, the deletion is a full-length deletion of the endogenous gene. In some embodiments, the gene, e.g., virulence gene, is genetically-altered to prevent transcription and/or translation of the gene encoding the protein. In some embodiments, the endogenous gene is genetically altered to insert a transcriptional terminator in the open reading frame of the gene. In some embodiments, a regulatory region of the gene, e.g., virulence gene, is genetically-modified to alter (e.g., decrease) the expression of the gene. In some embodiments, the promoter of a gene, e.g., virulence gene, is altered to include one or more regulatory elements (e.g., a sugar-responsive promoter).

As used herein, a “deletion mutation” or “deletion” in a gene refers to either partial or full-length deletion of a gene that abolishes activity of said gene. In one embodiment, a deletion is a partial deletion of a gene that abolishes activity of said gene. In another embodiment, a deletion is a full-length deletion of a gene that abolishes activity of said gene.

In some embodiments, the recombinant bacterium described herein is modified to comprise a nucleic acid comprising a gene. In some embodiments, the recombinant bacterium is modified to comprise a nucleic acid comprising a gene, whereby an endogenous copy of the gene in the bacterial chromosome has been altered and/or deleted. In some embodiments, the nucleic acid comprises a gene that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to an endogenous gene in the bacterial chromosome that has been deleted and/or altered. In some embodiments, the nucleic acid comprises a gene that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to an endogenous gene in the bacterial chromosome that has been deleted and/or altered. In some embodiments, the nucleic acid comprises a gene from a bacterial species, subspecies, serovar, or strain that is different than the bacterial species of the recombinant bacterium.

In some embodiments, the nucleic acid comprises a gene from a bacterial species, subspecies, serovar, or strain that is the same as the bacterial species of the recombinant bacterium. In some embodiments, the nucleic acid comprises a gene that is operably-linked to a regulatable promoter (e.g., a sugar-regulatable promoter). In some embodiments, the nucleic acid comprises a gene that is operably-linked to a rhamnose-regulatable promoter, a xylose-regulatable promoter, a galactose-regulatable promoter, an arabinose-regulatable promoter, a mannose-regulatable promoter, or a maltose-regulatable promoter. In some embodiments, the nucleic acid comprising the gene is located in a plasmid in the bacterium. In some embodiments, the nucleic acid comprising the gene is located in the bacterial chromosome. In some embodiments, the nucleic acid comprising the gene is located at the chromosomal locus corresponding to the locus of an endogenous gene that has been deleted or altered in the bacterial chromosome. In some embodiments, the nucleic acid is codon-optimized (e.g., to improve expression of the nucleic acid in the recombinant bacterium).

### 1. Lipopolysaccharide O-Antigen Conversion

In the case of interactions between animal host and bacterial pathogen, the bacterial surface is the first line of contact with the host immune system. Among the bacterial surface molecules undergoing pathogenic interactions, lipopolysaccharides (LPS) constitute the major structural component of the outer membrane of Gram-negative bacteria. The LPS fraction comprises about 10-15% of the total molecules in the outer membrane and is estimated to occupy about 75% of the bacterial surface [Lerouge and Vanderleyden, 2001].

LPS consists of three different components or regions: lipid A, core oligosaccharide, and O-polysaccharide. The structure of lipid A is highly conserved among Gram-negative bacteria; among Enterobacteriaceae, lipid A is virtually constant. Lipid A is the lipid component of LPS that contains the hydrophobic, membrane-anchoring region of LPS. The core oligosaccharide regions consist of a short chain of sugars, which connects the lipid A anchor to O-antigen, and can be divided into inner and outer core regions [Osborn and Wu, 1980].

LPS O-antigen has been reported to be a potent antigen for inducing specific antibody response which is protective against E. coli pathogens in animal models. The O-antigen or O-side chain is attached to the core polysaccharide and extends from the core out into the environment. It consists of repeating oligosaccharide subunits made up of three to five sugars. The individual chains can vary in length ranging up to 40 repeat units. The O-polysaccharide is much longer than the core oligosaccharide and it contains the hydrophilic domain of the LPS. At least 20 different sugars are known to occur and many of these sugars are characteristically unique dideoxyhexoses such as abequose, colitose, paratose, and tyvelose, which are rarely found elsewhere in nature. The composition of sugars in the 0-polysaccharide, especially terminal units, confers immunological specificity of the O-antigen and contributes to the wide variety of antigenic types (serotypes) between species and even strains of Gram-negative bacteria [Lerouge and Vanderleyden, 2001].

LPS O-antigen has been reported to be a potent antigen for inducing specific antibody response which is protective against E. coli pathogens in animal models [Pluschke and Achtman, 1985; Schief et al, 1993; Cryz Jr et al, 1995]. In addition, specific antibody against LPS O-antigen has been shown to be protective against other gram-negative pathogens [Zhang et al, 2009; Ding et al, 1990; Moustafa et al, 2023].

Multiple APEC serotypes have been associated with colibacillosis cases in the field outbreaks; however, three serotypes, O78, O2, and O1, account for the majority (more than 80%) of the cases [Dho-Moulin and Fairbrother, 1999; Ghunaim et al, 2014].

In some embodiments, the recombinant Salmonella bacterium provided herein expresses the LPS O78 antigen. The structure of O78 antigen of E. coli is known in the art and described in Jansson et al., “Structural studies of the Escherichia coli O78 O-antigen polyscharride”, Carbohydrate Research 165(1): 87-92 (1987), and Liu et al., “Structure and genetics of Escherichia coli O antigens”, FEMS Microbiol Rev. 44(6): 655-683 (2020), the entire contents of which are incorporated herein by reference.

In some embodiments, the O78 antigen comprises the following structure:
→4)-DLcNAc-(β1→4)-Dman-(β1→)-DMan-(α1→3)-DGlcNAc-(β1→,
wherein the sugar residues in this structure are D-N-Acetylglucosamine (D-GlcNAc) and D-mannose (D-Man) which are linked by ‘1→4 or α1→3 linkages.

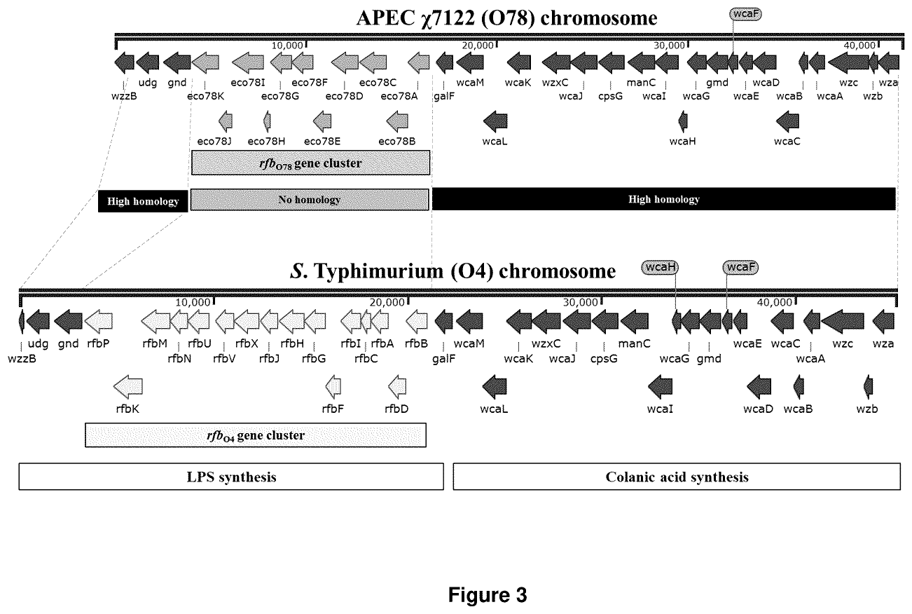

O78 antigen synthesis is carried out by a number of genes in the O78-gene cluster rfb_{O78}: eco78ABCDEFGHIJK depicted in FIG. 3, also referred to herein as the rfb_{O78} gene cluster. The O78-gene cluster rfb_{O78} is about 13 kb long. In some embodiments, the O78-gene cluster rfb_{O78} is located between the galF and gnd genes in the chromosome of APEC strain X⁷¹²².

In some embodiments, the recombinant Salmonella bacterium provided herein comprises all O78 antigen synthesis genes. In some embodiments, the recombinant bacterium comprises the O78-gene cluster rfb_{O78}.

In some embodiments, the rfb_{O78} gene cluster comprises a nucleic acid sequence having at least 50%, at least 65%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 4. In one embodiment, the rfb_{O78} gene cluster comprises a nucleic acid sequence comprising SEQ ID NO: 4. In one embodiment, the rfb_{O78} gene cluster comprises a nucleic acid sequence consisting of SEQ ID NO: 4.

In some embodiments, the recombinant Salmonella bacterium does not express the Salmonella LPS O4 antigen. The structure of O4 antigen of Salmonella typhirium is known in the art and described in Liu et al., “Structural diversity in Salmonella O antigens and its genetic basis”, FEMS Microbiol Rev 38: 56-89 (2014), the entire content of which is incorporated herein by reference.

In some embodiments, the O4 antigen comprises the following structure:
→2)-_{D}-Man-(α1→4)-_{L}-Rha-(α1→3)-_{D}-Gal-(α1→
   wherein the sugar residues in this structure are D-mannose (_{D}-Man), _{L}-rhamnose (_{L}-Rha), and _{D}-galactose (_{D}-Gal) which are linked by α1→4 or α1→3 linkages.

O4 antigen synthesis is carried out by a number of genes in the O4-gene cluster rfb_{O4} depicted in FIG. 3, also referred to herein as the rfb_{O4} gene cluster. The O4-gene cluster rfb_{O4} is about 18 kb long. In some embodiments, the O4-gene cluster rfb_{O4} is located between the galF and gnd genes in the chromosome of Salmonella bacterium.

In some embodiments, the recombinant Salmonella bacterium provided herein comprises a deletion mutation in one or more O4 antigen synthesis genes such that O4 antigen synthesis is abolished. In some embodiments, the recombinant bacterium comprises a deletion mutation in the O4-gene cluster rfb_{O4}.

In some embodiments, the APEC O78-gene cluster rfb_{O78} replaces the entire Salmonella O4-gene cluster rfb_{O4} between the galF and gnd genes in the recombinant Salmonella bacterium.

In some embodiments, the recombinant bacterium comprises genetic features or modification that enhance expression of O78 antigen and/or enhance immunogenicity of the bacterium.

In some embodiments, the recombinant bacterium comprises a phosphomannose isomerase, or pmi, gene (pmi+). In some embodiments, the pmigene comprises a nucleic acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 11. In one embodiment, the pmigene has a nucleic acid sequence comprising SEQ ID NO: 11. In another embodiment, the pmigene has a nucleic acid sequence that consists of SEQ ID NO: 11. In some embodiments, the endogenous pmigene is not deleted in the bacterium. In some other embodiments, a heterologous wild-type pmi gene is added to the bacterium. The presence of the pmi gene enables the recombinant bacterium to be mannose-independent. The nucleic acid sequence of an exemplary Salmonella pmi gene is provided below:

(SEQ ID NO: 11)
atgcaaaaactcattaactcagtgcaaaactatgcctggggaagtaaaa
ctgcgttaacggaactttatggcatcgccaatccgcagcagcagccaat
ggctgaactctggatgggcgcgcatcccaaaagcagctcgcgaatcacc
accgccaacggcgaaaccgtctccctgcgtgacgccatcgaaaagaata
aaaccgccatgctgggcgaagcggtagccaaccgtttcggcgaactgcc
gtttctgtttaaagtactgtgcgccgcacaaccgctctctattcaggtg
cacccgaataaacgcaactccgaaatcggtttcgcgaaagaaaatgcgg
cgggtatccccatggatgccgcagagcggaactataaagatcctaacca
taaaccagagctggtttttgccctgacgcctttcctggcgatgaacgcg
ttccgcgaattttctgacattgtctctttactgcaacctgtcgccggcg
cgcattccgctatcgcccactttttgcaggtgccgaatgctgaacgtct
gagccagcttttcgccagcctgttgaatatgcaaggcgaagaaaaatcc
cgcgcgttagccgtactcaaagcggcgcttaacagccagcaaggcgaac
cgtggcaaacgatccgcgtgatttcagagtattatcctgacgacagcgg
gcttttctctcctttgttgctgaatgtggtcaaactgaatcccggcgag
gcgatgttcctgtttgctgaaacgcctcatgcttatctgcagggcgttg
cgctggaagtcatggcgaactccgataacgttctgcgcgctggccttac
gccaaaatatatcgacatccctgagctggtcgcgaacgtgaagttcgaa
cctaagcctgccggcgagttgctgactgccccggtgaaaagcggcgcgg
agctggacttcccaattccggttgacgattttgctttttcactgcacga
cctggcgcttcaggagacgagcatcggccaacacagcgccgcgattctg
ttctgcgttgagggtgaggcggtgttacgtaaagatgaacagcgtctgg
tactgaagccgggtgaatctgcctttatcggcgcggatgagtctccggt
taacgccagcggcacgggccgtttagcgcgtgtttataacaagctgtag.

In some embodiments, the recombinant bacterium comprises a deletion in the leucine-responsive regulatory (lrp) gene. The Lrp protein appears to be an antivirulence regulator in Salmonella since the deletion mutant (Δlrp) showed enhanced cell invasion, cytotoxicity, and hypervirulence. The lrp gene and methods for creating the deletion mutation Δlrp is described in the art, such as in Baek et al., “Leucine-responsive regulatory protein (Lrp) acts as a virulence repressor in Salmonella enterica serovar Typhimurium.” J Bacteriol 191:1278-1292, incorporated herein by reference in its entirety.

Any methods known in the art can be used to determine whether a recombinant Salmonella bacterium express O78 or O4 antigen. In a non-limiting embodiments, expression of O78 and O4 can be determined using anti-O78 and anti-O4 antibody, as demonstrated in Examples 2-3 of the present disclosure.

### 2. APEC Virulence Factors

Avian pathogenic E. coli (APEC) is one type of extraintestinal pathogenic E. coli (ExPEC) pathotype [Russo and Johnson, 2000]. APEC causes diverse systemic infections in poultry including chickens, turkeys, ducks, and many other avian species [Barnes et al, 2003; Dho-Moulin and Fairbrother, 1999]. The most common diseases caused by APEC in chickens collectively known as colibacillosis infections including airsacculitis, cellulitis, coligranuloma, egg peritonitis, omphalitis, osteomyelitis/arthritis, perihepatitis, pericarditis, and salphingitis [Dziva and Stevens, 2008]. Colibacillosis caused by APEC is a devastating disease of poultry that results in severe economic losses annually to the poultry industry worldwide [Newman et al., 2021].

The most prevalent virulence traits associated with APEC are adhesins (type 1 and P fimbriae, Temperature-sensitive hemagglutinin Tsh, and curli), capsules, iron acquisition systems, and serum resistance [Dho-Moulin and Fairbrother, 1999]. Large plasmids are commonly found in APEC strains and are associated with the virulence of APEC [Dozois et al, 2000; Doetkott et al, 1996; Rodriguez-Siek et al, 2005]. Their importance has been demonstrated by the occurrence of plasmid-encoded genes shared among APEC strains [Dozois et al, 2003] and the presence of virulence genes in plasmids [Johnson et al, 2002; Stehling et al, 2003]. Most virulence genes associated with APEC are often located on IncF plasmids, named CoIV plasmids because their ability to code for production of colicin V, a small protein from the microcine family [Johnson et al, 2002; Johnson et al, 2004; Tivendale et al, 2004]. CoIV plasmids are associated with E. coli in general and with APEC in particular. Their link with the ability of bacteria to cause disease in production animals has been demonstrated [Skyberg et al, 2006; Smith et al, 1980; Wooley et al, 1998]. In vivo expression of genes encoded by the ColV plasmid, including Tsh, aerobactin, and the Iro system, has been confirmed by using SCOTS [Dozois et al, 2003] and the occurrence of the putative virulence genes of APEC on non-CoIV plasmids has also been shown [Dozois et al, 2003; Johnson et al, 2006; Dozois et al, 2000].

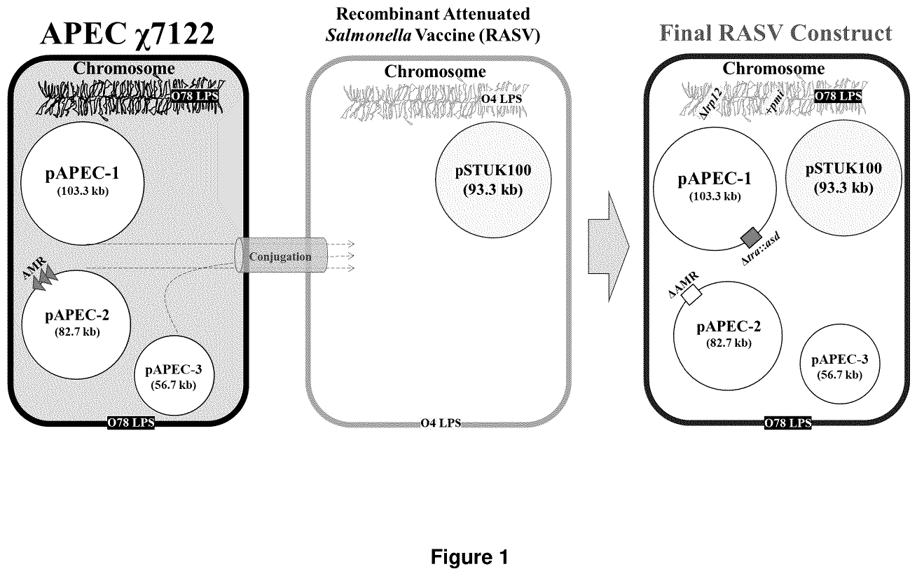

The complete nucleotide sequence analysis of the large CoIV/IncF virulence plasmid pAPEC-1 (103,275-bp) from the APEC strain X⁷¹²² (serogroup O78:K80:H9) revealed that a putative virulence region spanning an 80-kb region of pAPEC-1 comprises genes responsible for the potential virulence factors including four iron acquisition systems (iutA, iucABCD, sitABCD, and iroBCDN), temperature-sensitive hemagglutinin (tsh), a colicin V operon, increasing serum sensitivity (iss), ompT, hlyF, and etsABC [Mellata et al, 2009]. In addition, it has been reported that pAPEC-1-cured X⁷¹²² bacteria were attenuated in chickens, caused few lesions of pericarditis and perihepatitis, did not persist in the blood, and poorly colonized the lung, spleen, and liver [Dozois et al, 2000]. This APEC strain X⁷¹²² has two additional large plasmids (FIG. 1), pAPEC-2 (82,676-bp) and pAPEC-3 (56,676-bp), that also have virulence attributes [Mellata et al, 2010].

In some embodiments, the recombinant Salmonella bacterium disclosed herein comprises one or more nucleotide encoding one or more virulence factor of APEC.

In some embodiments, the recombinant Salmonella bacterium comprises one or more APEC plasmids that comprises at least one gene encoding a virulence factor of APEC.

In some embodiments, the APEC plasmid is a pAPEC-1, a pAPEC-2, or a pAPEC-3 plasmid of APEC strain X⁷¹²². As used here in, pAPEC-1, pAPEC-2 and pAPEC-3 plasmids refer to the original pAPEC-1, pAPEC-2, or pAPEC-3 plasmids of APEC strain X⁷¹²², respectively, as well as plasmids derived from the original pAPEC-1, pAPEC-2, or pAPEC-3 plasmids of APEC strain X⁷¹²².

In some embodiments, the recombinant Salmonella bacterium comprises a pAPEC-1, a pAPEC-2, a pAPEC-3 plasmid, or a combination thereof. In some embodiments, the recombinant Salmonella bacterium comprises both pAPEC-1, pAPEC-2, and pAPEC-3 plasmids.

In some embodiments, the pAPEC-1 plasmid comprises a Δtra::asd mutation, which replaces the tra (traM-traX) genes on pAPEC-1 with asd gene. In some embodiments the pAPEC-1 plasmid comprises the nucleic acid sequence of SEQ ID NO: 1. In some embodiments the pAPEC-1 plasmid comprises a nucleic acid sequence having at least 50%, at least 65%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1. In some embodiments the pAPEC-1 plasmid comprises a nucleic acid sequence consisting of SEQ ID NO: 1.

In some embodiments, the pAPEC-2 plasmid has the antimicrobial resistance (AMR) genes removed. In some embodiments, the pAPEC-2 plasmid comprises the nucleic acid sequence of SEQ ID NO: 2. In some embodiments, the pAPEC-2 plasmid comprises a nucleic acid sequence consisting of SEQ ID NO: 2. In some embodiments, the pAPEC-2 plasmid comprises a nucleic acid sequence having at least 50%, at least 65%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2.

In some embodiments, the pAPEC-3 plasmid comprises the nucleic acid sequence of SEQ ID NO: 3. In some embodiments, the pAPEC-3 plasmid comprises a nucleic acid sequence consisting of SEQ ID NO: 3. In other embodiments, the pAPEC-3 plasmid comprises a nucleic acid sequence having at least 50%, at least 65%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3.

### 3. Recombinant Attenuated Salmonella

The recombinant Salmonella bacterium provided herein further comprises additional modifications which enable it to effectively express the O78 antigen and virulence factors of APEC. In some embodiments, the recombinant bacterium is particularly effective in eliciting an immune response (e.g., protective immunity) against the pathogen of interest because the bacterium comprise multiple recombinant regulatory systems that permit the bacterium to replicate upon administration and to colonize lymphoid tissues in a subject in order to elicit potent immune responses. In other embodiments, the recombinant bacterium provided herein may ultimately exhibits an attenuated phenotype after multiple replication cycles in vivo, which allows for safe administration to a subject, for example, as a vaccine composition.

Recombinant attenuated Salmonella strains described in that art can be used to generate the recombinant Salmonella bacterium of the present disclosure. Examples for recombinant attenuated Salmonella strains may be found at least in U.S. Pat. No. 10,988,729, incorporated herein by reference in its entirety.

The recombinant regulatory systems of the bacteria described herein depend, in part, on multiple genetic regulatory elements that are responsive to one or more sugars (e.g., arabinose, rhamnose, mannose, maltose, xylose, and galactose) that not available to the bacterium in vivo. Thus, the phenotype of the recombinant bacteria described herein can be altered upon administration to a subject. In some embodiments, the subject is administered one or more sugars before, after or concurrently with the administration of a recombinant bacterium described herein in order to activate and/or repress a sugar-responsive regulatory system of the bacteria. In some embodiments, the recombinant bacterium described herein comprises at least three regulatory systems, each dependent on a different sugar, which facilitates initial invasion of a host cell in the subject, delayed attenuation, and improved immunogenicity.

In some embodiments, the recombinant bacterium described herein can be regulated for delayed attenuation in vivo. In some embodiments, the recombinant bacterium described herein exhibits regulated production of Generalized Modules for Membrane Antigens (GMMA), or outer membrane vesicles, in vivo, which may lead to enhanced production of conserved outer membrane proteins present in the bacterium, and ultimately improved immunogenicity.

Some embodiments of the instant disclosure comprise a species or subspecies of the Salmonella genera (e.g., S. enterica or S. bongorn). For instance, the recombinant bacterium may be a Salmonella enterica serovar, including, for example, Paratyphi A, Enteritidis, Typhi, and Typhimurium. In some embodiments, the recombinant bacterium is of the serovar S. typhimurium, S. Typhi, S. Paratyphi, S. Gallinarum, S. Enteritidis, S. Choleraesius, S. Arizonae, S. Newport, S. Heidelberg, S. Infantis, S. Cholerasiuis, or S. Dublin.

A recombinant bacterium derived from Salmonella may be particularly suited to use as a vaccine. For example, oral infection of a host with a Salmonella strain typically leads to colonization of the gut-associated lymphoid tissue (GALT) or Peyer's patches, which leads to the induction of a generalized mucosal immune response to the recombinant bacterium. Further penetration of the bacterium into the mesenteric lymph nodes, liver and spleen may augment the induction of systemic and cellular immune responses directed against the bacterium. Thus, the use of recombinant Salmonella for oral immunization stimulates all three branches of the immune system, which is particularly important for immunizing against infectious disease agents that colonize on and/or invade through mucosal surfaces.

In some embodiments, the recombinant bacterium comprises a deletion in an endogenous O-antigen synthesis gene. In some embodiments, the recombinant bacterium comprises a deletion in an endogenous O-antigen ligase gene. In some embodiments, the deletion is a partial deletion of the endogenous O-antigen ligase gene. In some embodiments, the deletion is a full-length deletion of the endogenous O-antigen ligase gene. In some embodiments, the endogenous O-antigen ligase gene is genetically altered to insert a transcriptional terminator in the open reading frame of the gene. In some embodiments, a regulatory region of the endogenous O-antigen ligase gene is genetically-modified to alter (e.g., decrease) the expression of the gene. In some embodiments, the promoter of a endogenous 0-antigen ligase gene is altered to include one or more regulatory elements (e.g., a sugar-responsive promoter). In some embodiments, the promoter of a endogenous O-antigen ligase gene is altered to increase the spacing between the Shine-Delgarno sequence and the start codon of the gene. In some embodiments, the promoter of a endogenous O-antigen ligase gene is altered to decrease the spacing between the Shine-Delgarno sequence and the start codon of the gene. In some embodiments, the Shine-Delgarno (SD) sequence, the start codon, the second codon and/or third codons of the O-antigen ligase gene is altered to increase the frequency of adenine nucleobases in order to enhance the translation efficiency of the gene. In some embodiments, the Shine-Delgarno (SD) sequence, the start codon, the second codon and/or third codons of the O-antigen ligase gene is altered to reduce the frequency of adenine nucleobases in order to decrease the translation efficiency of the gene. In some embodiments, the O-antigen ligase gene is waaL (also known as rfaL). The O-antigen ligase WaaL is necessary to ligate polysaccharide to the lipid A-LPS core moiety. Deletion of waaL results in an intact lipid A-LPS core with no O-antigen or individual sugars attached to it. In some embodiments, the O-antigen ligase gene is selected from the group consisting of waaG (also known as rfaG), waaI (also known as rfaI), rfaH, waaJ (also known as rfaJ, wbaP (also known as rfbP), wzy (also known as rfc), waaP, waaQ, waaF, waaP, waaC, and waaA.

In some embodiments, the recombinant bacterium described herein is modified to comprise a nucleic acid comprising an O-antigen ligase gene. In some embodiments, the nucleic acid comprising an O-antigen ligase gene is located on a plasmid in the bacterium. In some embodiments, the nucleic acid comprising an O-antigen ligase gene is located on a chromosome of the bacterium. In some embodiments, the nucleic acid comprising an O-antigen ligase gene is located at the chromosomal locus corresponding to the locus of an endogenous O-antigen ligase gene that has been deleted or altered in the bacterial chromosome. In some embodiments, the recombinant bacterium is modified to comprise a nucleic acid comprising an O-antigen ligase gene, whereby an endogenous copy of the gene in the bacterial chromosome has been altered and/or deleted. In some embodiments, the nucleic acid comprises a Salmonella O-antigen ligase gene.

The nucleic acid sequence of an exemplary Salmonella waaL gene is provided below:

(SEQ ID NO: 5)
atgctaaccacatcattaacgttaaataaagagaaatggaagccgatct
ggaataaagcgctggtttttctttttgttgccacgtattttctggatgg
tattacgcgttataaacatttgataatcatacttatggttatcaccgcg
atttatcaggtctcacgctcaccgaaaagtttcccccctcttttcaaaa
atagcgtattttatagcgtagcagtattatcattaatccttgtttattc
catactcatatcgccagatatgaaagaaagtttcaaggaatttgaaaat
acggtactggagggcttcttattatatactttattaattcccgtactat
taaaagatgaaacaaaagaaacggttgcgaaaatagtacttttctcctt
tttaacaagtttaggacttcgctgccttgcagagagtattctgtatatc
gaggactataataaagggattatgccattcataagctatgcgcatcgac
atatgtccgattccatggttttcttatttccagcattattgaatatttg
gctgtttagaaaaaatgcaattaagttggtttttttggtgcttagcgcc
atctaccttttctttatcctgggaaccctatcgcgaggggcatggttgg
cggtgcttatagtaggtgttctgtgggcaatactgaaccgccaatggaa
gttaataggagttggtgccattttattagccattatcggcgctttggtt
atcactcaacataataacaaaccagacccagaacatttactgtataaat
tacagcagacagatagctcatatcgttatactaacggaacccagggcac
cgcgtggatactgattcaggaaaacccgatcaagggctacggctatggt
aatgatgtgtatgatggtgtttataataaacgcgttgtcgattatccaa
cgtggacctttaaagaatctatcggtccgcataataccattctgtacat
ctggtttagtgcaggcatattgggtctggcgagcctggtctatttatat
ggcgctatcatcagggaaacagccagctctaccctcaggaaagtagaga
taagcccctacaatgctcatctcttgctatttttatctttcgtcggttt
ttatatcgttcgtggcaattttgaacaggtcgatattgctcaaattggt
atcattaccggttttctgctggcgctaagaaatagataa.

The amino acid sequence of the WaaL protein encoded by the nucleic acid of SEQ ID NO: 5 is provided below:

(SEQ ID NO: 6)
MLTTSLTLNKEKWKPIWNKALVFLFVATYFLDGITRYKHLIIILMVITA
IYQVSRSPKSFPPLFKNSVFYSVAVLSLILVYSILISPDMKESFKEFEN
TVLEGFLLYTLLIPVLLKDETKETVAKIVLFSFLTSLGLRCLAESILYI
EDYNKGIMPFISYAHRHMSDSMVFLFPALLNIWLFRKNAIKLVFLVLSA
IYLFFILGTLSRGAWLAVLIVGVLWAILNRQWKLIGVGAILLAIIGALV
ITQHNNKPDPEHLLYKLQQTDSSYRYTNGTQGTAWILIQENPIKGYGYG
NDVYDGVYNKRVVDYPTWTFKESIGPHNTILYIWFSAGILGLASLVYLY
GAIIRETASSTLRKVEISPYNAHLLLFLSFVGFYIVRGNFEQVDIAQIG
IITGFLLALRNR.

In some embodiments, the nucleic acid comprises a Salmonella waaL gene (provided as SEQ ID NO: 5). In some embodiments, the nucleic acid comprises a waaL gene, wherein the waaL gene comprises a nucleic acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 5. In some embodiments, the nucleic acid comprises a waaL gene, wherein the waaL gene comprises a nucleic acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to the nucleic acid sequence of SEQ ID NO: 5.

In some embodiments, the nucleic acid comprises a nucleic acid sequence encoding an O-antigen ligase, wherein said O-antigen ligase comprises an amino acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO: 6. In some embodiments, the nucleic acid comprises a nucleic acid sequence encoding an O-antigen ligase, wherein said 0-antigen ligase comprises an amino acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to the nucleic acid sequence of SEQ ID NO: 6.

In some embodiments, the nucleic acid comprises an O-antigen ligase gene from a bacterial species, subspecies, serovar, or strain that is different than the bacterial species of the recombinant bacterium. In some embodiments, the nucleic acid comprises an O-antigen ligase gene from a bacterial species, subspecies, serovar, or strain that is the same as the bacterial species of the recombinant bacterium.

In some embodiments, the nucleic acid comprises an O-antigen ligase gene that is operably-linked to a regulatable promoter (e.g., a sugar-regulatable promoter). In some embodiments, the nucleic acid comprises an O-antigen ligase gene (e.g., waaL) that is operably-linked to a sugar-regulatable promoter. Advantageously, recombinant bacterial strains comprising a nucleic acid comprising an O-antigen ligase gene (e.g., waaL) that is operably linked to a sugar regulatable promoter will synthesize normal LPS in the presence of the sugar (e.g., rhamnose) in vitro, but will form rough LPS in vivo due to the absence of the sugar that activates the promoter and therefore, the expression of the O-antigen ligase. Without wishing to be bound by any particular theory, using this strategy, the bacterium will expose conserved LPS core oligosaccharide and have enhanced production of conserved outer membrane proteins (OMPs; e.g., porins) which may lead to improved immunogenicity and aid in the production of a cross-protective immune response against an antigen of interest synthesized in the bacterium in vivo. In some embodiments, the sugar regulatable promoter exhibits increased activity (e.g., increased transcription) in the presence of a specific sugar and decreased activity in the absence of a sugar. In some embodiments, the nucleic acid comprises an O-antigen ligase gene that is operably-linked to a rhamnose-regulatable promoter (e.g., a sugar-regulatable promoter). In some embodiments, the nucleic acid comprises an O-antigen ligase gene that is operably-linked to an arabinose-regulatable promoter (e.g., a sugar-regulatable promoter). In some embodiments, the use of a rhamnose-regulatable promoter (e.g., rhaSR P_{rhaBAD}) may be preferable to an arabinose-regulatable promoter because a relatively higher concentration is required to activate an arabinose-regulatable promoter as compared to a rhamnose-regulatable promoter (see, e.g., Giacalone et al. (2006) BioTechniques 40(3): 355-366 (39), the entire contents of which are incorporated herein by reference). In some embodiments, the recombinant bacterium comprises the mutation ΔwaaL/ΔpagL::TT rhaSR P_{rhaBAD} waaL.

In some embodiments, the recombinant bacterium comprises a deletion in an endogenous lipid A deacylase gene. In some embodiments, the deletion is a partial deletion of the endogenous lipid A deacylase gene. In some embodiments, the deletion is a full-length deletion of the endogenous lipid A deacylase gene. In some embodiments, the endogenous lipid A deacylase gene is genetically altered to insert a transcriptional terminator in the open reading frame of the gene. In some embodiments, a regulatory region of the endogenous lipid A deacylase gene is genetically-modified to alter (e.g., decrease) the expression of the gene. In some embodiments, the promoter of an endogenous lipid A deacylase gene is altered to include one or more regulatory elements (e.g., a sugar-responsive promoter). In some embodiments, the lipid A deacylase gene is pagL. Bacterial comprising a deletion of the lipid A deacylase gene pagL have been found to produced increased amounts of outer membrane vesicles (see, e.g., Elhenawy et al. (2016) mBio 7(4): e00940-16 (40)). Deletion of the pagL gene of Salmonella does not impair bacterial virulence (see, e.g., Man et al. Proc. Nat'l. Acad. Sci. USA 111: 7403-8 (41)). Without wishing to be bound by any particular theory, in some embodiments, the recombinant bacterium described herein comprise one or more genetic modifications which results in increased vesiculation (i.e., increased vesicle production) which may be particularly advantageous in inducing an immune response in the host against an antigen of interest that is expressed by the bacterium.

In some embodiments, the recombinant bacterium comprises a deletion in an endogenous phosphomannose isomerase gene. Phosphomannose isomerase, also known as mannose-6 phosphate isomerase, catalyzes the reversible interconversion of fructose 6-phosphate to mannose 6-phosphate. Mannose 6-phosphate is then converted to GDP-mannose and used for the synthesis of O-antigen side chains. Bacteria with deletions of the phosphomannose isomerase gene pmi are not mannose sensitive and are partially attenuated (see, e.g., Collins et al. (1991) Infect. Immun. 59(3): 1079-85 (42)). These pmi mutants synthesize wild-type levels of LPS O-antigen side chains when grown in media containing mannose, and are both attenuated but highly immunogenic (see, e.g., Curtiss et al. (2007) “Induction of host immune responses using Salmonella-vectored vaccines.” In: Brogden K A, Minion F C, Cornick N, Stanton T B, Zhang Q, Nolan L K, Wannemuehler M J, ed. Virulence Mechanisms of Bacterial Pathogens. 4th ed. Washington DC: ASM Press (43)). In some embodiments, the deletion of the endogenous phosphoisomerase gene is a partial deletion. In some embodiments, the deletion of the endogenous phosphomannose isomerase gene is a full-length deletion. In some embodiments, the endogenous phosphomannose isomerase gene is genetically altered to insert a transcriptional terminator in the open reading frame of the gene. In some embodiments, a regulatory region of the endogenous phosphomannose isomerase gene is genetically-modified to alter (e.g., decrease) the expression of the phosphomannose isomerase gene. In some embodiments, the promoter of an endogenous phosphomannose isomerase gene is altered to include one or more regulatory elements (e.g., a sugar-responsive promoter). In some embodiments, the phosphomannose isomerase gene is pmi.

In some embodiments, the bacterium comprises a deletion of a pmigene. In some embodiments, the bacterium comprises a deletion in the pmigene (Δpmi), e.g., Δpmi-2426 mutation. A bacterium comprising a Δpmi-2426 mutation, grown in the presence of mannose, is capable of synthesizing a complete LPS O-antigen. Non-phosphorylated mannose, which is the form required for bacterial uptake, is unavailable in vivo. Hence, a bacterium comprising a Δpmi-2426 mutation loses the ability to synthesize LPS O-antigen serotype specific side chains in vivo and the number of O-antigen side chains attached to the LPS core decreases by about half after each cell division in vivo. The LPS that is synthesized comprises a core structure that is substantially similar across all Salmonella enterica serotypes except S. Arizona. This results in a bacterium that is capable of eliciting an immune response against at least two Salmonella serotypes without substantially inducing an immune response specific to the serotype of the bacterial vector. In some embodiments, the bacterium is capable of eliciting an immune response against all Salmonella serotypes without substantially inducing an immune response specific to the serotype of the bacterial vector.

A recombinant bacterium described herein that comprises a deletion in a pmi mutation may also comprise other mutations that ensure that mannose available to the bacterium during in vitro growth is used for LPS O-antigen synthesis. For instance, a bacterium may comprise a deletion in the gmd-fcl genes (Δ(gmd-fcl)), e.g., Δ(gmd-fcl)-26 mutation. This mutation deletes two nucleic acid sequences that encode enzymes for conversion of GDP-mannose to GDP-fucose, ensuring that mannose available to the bacterium during in vitro growth is used for LPS O-antigen synthesis and not colanic acid production. Similarly, a bacterium may comprise a deletion in the wcaM-wza gene cluster (Δ(wcaM-wza)) e.g., the Δ(wcaM-wza)-8 mutation, which deletes all 20 nucleic acid sequences necessary for colanic acid production, and also precludes conversion of GDP-mannose to GDP-fucose.

In some embodiments, the recombinant bacterium has been genetically-altered such that the bacterium is capable of escaping the endosomal compartment of a host cell. A recombinant bacterium may exhibit a temporal delay in escaping an endosome following invasion of the host cell. Methods of detecting escape from an endosomal compartment of a host cell are well known in the art, and include, for example, microscopic analysis.

In some embodiments, the recombinant bacterium comprises a deletion in an endogenous sifA gene. In some embodiments, the recombinant bacterium comprises a mutation that alters the function of SifA. SifA is an effector protein necessary for the formation of Salmonella-induced filaments and for the maintenance of the vacuolar membrane enclosing the bacterium. Bacteria comprising a deletion of sifA are capable of escaping the host cell endosome (also called the Salmonella-containing vesicle, or SCV) following cellular invasion. In some embodiments, the deletion of the endogenous sifA gene is a partial deletion. In some embodiments, the deletion of the endogenous sifA gene is a full-length deletion. In some embodiments, the endogenous sifA gene is genetically altered to insert a transcriptional terminator in the open reading frame of the gene. In some embodiments, a regulatory region of the endogenous sifA gene is genetically-modified to alter (e.g., decrease) the expression of the sifA gene. In some embodiments, the promoter of an endogenous sifA gene is altered to include one or more regulatory elements (e.g., a sugar-responsive promoter).

In some embodiments, the recombinant bacterium described herein is modified to comprise a nucleic acid comprising a sifA gene. In some embodiments, the nucleic acid comprising a sifA gene is located on a plasmid in the bacterium. In some embodiments, the nucleic acid comprising a sifA gene is located on a chromosome of the bacterium. In some embodiments, the nucleic acid comprising a sifA gene is located at the chromosomal locus corresponding to the locus of an endogenous a sifA that has been deleted or altered in the bacterial chromosome. In some embodiments, the recombinant bacterium is modified to comprise a nucleic acid comprising a sifA gene, whereby an endogenous copy of the sifA gene in the bacterial chromosome has been altered and/or deleted.

The nucleic acid sequence of an exemplary Salmonella sifA gene is provided below:

(SEQ ID NO: 7)
atgccgattactatagggaatggttttttaaaaagtgaaatccttacca
actccccaaggaatacgaaagaagcatggtggaaagttttatgggaaaa
aattaaagacttctttttttctactggcaaagcaaaagcggaccgttgt
ctacatgagatgttgtttgccgaacgcgcccccacacgagagcggctta
cagagattttttttgagttgaaagagttagcctgcgcatcgcaaagaga
tagatttcaggttcataatcctcatgaaaatgatgccaccattattctt
cgcatcatggatcaaaacgaagagaacgaattgttacgtatcactcaaa
ataccgatacctttagctgtgaagtcatggggaatctttattttttaat
gaaagatcgcccggatattttaaaatcgcatccacaaatgacggccatg
attaagagaagatatagcgaaatcgtagactaccccctcccttcgacat
tatgtctcaatcctgctggcgcgccgatattatcggttccattagacaa
catagaggggtatttatatactgaattgagaaaaggacatttagatggg
tggaaagcgcaagaaaaggcaacctacctggcagcgaaaattcagtctg
ggattgaaaagacaacgcgcattttacaccatgcgaatatatccgaaag
tactcagcaaaacgcatttttagaaacaatggcgatgtgtggattaaaa
cagcttgaaataccaccaccgcatacccacatacctattgaaaaaatgg
taaaagaggttttactagcggataagacgtttcaggcgttcctcgtaac
ggatcccagcaccagccaaagtatgttagctgagatagtcgaagccatc
tctgatcaggtttttcacgccatttttagaatagacccccaggctatac
aaaaaatggcggaagaacagttaaccacgctacacgttcgctcagaaca
acaaagcggctgtttatgttgttttttataa.

The amino acid sequence of the SifA protein encoded by the nucleic acid of SEQ ID NO: 7 is provided below:

(SEQ ID NO: 8)
MPITIGNGFLKSEILTNSPRNTKEAWWKVLWEKIKDFFFSTGKAKADRC
LHEMLFAERAPTRERLTEIFFELKELACASQRDRFQVHNPHENDATIIL
RIMDQNEENELLRITQNTDTFSCEVMGNLYFLMKDRPDILKSHPQMTAM
IKRRYSEIVDYPLPSTLCLNPAGAPILSVPLDNIEGYLYTELRKGHLDG
WKAQEKATYLAAKIQSGIEKTTRILHHANISESTQQNAFLETMAMCGLK
QLEIPPPHTHIPIEKMVKEVLLADKTFQAFLVTDPSTSQSMLAEIVEAI
SDQVFHAIFRIDPQAIQKMAEEQLTTLHVRSEQQSGCLCCFL.

In some embodiments, the nucleic acid comprises a Salmonella sifA gene (provided as SEQ ID NO: 7). In some embodiments, the nucleic acid comprises a sifA gene, wherein the sifA gene comprises a nucleic acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 7. In some embodiments, the nucleic acid comprises a sifA gene, wherein the sifA gene comprises a nucleic acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to the nucleic acid sequence of SEQ ID NO: 7.

In some embodiments, the nucleic acid comprises a nucleic acid sequence encoding a SifA protein, wherein said SifA protein comprises an amino acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO: 8. In some embodiments, the nucleic acid comprises a nucleic acid sequence encoding a SifA protein, wherein said SifA protein comprises an amino acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to the nucleic acid sequence of SEQ ID NO: 8.

In some embodiments, the nucleic acid comprises a sifA gene from a bacterial species, subspecies, serovar, or strain that is different than the bacterial species of the recombinant bacterium. In some embodiments, the nucleic acid comprises a sifA gene from a bacterial species, subspecies, serovar, or strain that is the same as the bacterial species of the recombinant bacterium.

In some embodiments, the nucleic acid comprises a sifA gene that is operably-linked to a regulatable promoter (e.g., a sugar-regulatable promoter). In some embodiments, the nucleic acid comprises a sifA gene that is operably-linked to a sugar-regulatable promoter. In some embodiments, the sugar regulatable promoter exhibits increased activity (e.g., increased transcription) in the presence of a specific sugar and decreased activity in the absence of a sugar. In some embodiments, the nucleic acid comprises a sifA gene that is operably-linked to a rhamnose-regulatable promoter (e.g., a sugar-regulatable promoter). In some embodiments, the nucleic acid comprises a sifA gene that is operably-linked to an arabinose-regulatable promoter. In some embodiments, the arabinose-regulatable promoter is P_{araBAD}. In some embodiments, the recombinant bacterium comprises the mutation ΔsifA::TT araC P_{araBAD} sifA. In some embodiments, the recombinant bacterium comprises the mutation ΔP_{sifA}::TT araC P_{araBAD} sifA. When the expression of the nucleic acid comprising a sifA gene is under the control of an arabinose-regulated promoter, the bacterial escape from the host endosome can be delayed. Since arabinose is absent in host cells, arabinose cannot induce the expression of the sifA gene. Thus, if the recombinant bacterium is cultured in the presence of arabinose prior to administration to the subject, the expression of sifA will gradually decrease with each round of bacterial cell division thereby allowing escape of the bacterium from the host cell endosome during the initial cell division cycles. Similar delayed-escape mutations may be constructed using other regulatable promoters, such as from the xylose-regulatable or rhamnose-regulatable promoter systems.

In some embodiments, the recombinant bacterium comprises a deletion in an endogenous relA gene, which encodes the GTP pyrophosphokinase RelA. The inclusion of a relA deletion in the recombinant bacterium uncouples the occurrence of growth-dependent lysis to the need for continued protein synthesis. In some embodiments, the deletion of the endogenous relA gene is a partial deletion. In some embodiments, the deletion of the endogenous relA gene is a full-length deletion.

Other methods of attenuation are known in the art. For instance, attenuation may be accomplished by altering (e.g., deleting) native nucleic acid sequences found in the wild-type bacterium. For instance, if the bacterium is Salmonella, non-limiting examples of nucleic acid sequences which may be used for attenuation include: a pab nucleic acid sequence, a pur nucleic acid sequence, an aro nucleic acid sequence, asd, a dap nucleic acid sequence, nadA, pncB, galE, pmi, fur, rpsL, ompR, htrA, hemA, cdt, cya, crp, dam, phoP, phoQ, rfc, poxA, galU, mviA, sodC, recA, ssrA, sirA, inv, hilA, rpoE, flgM, tonB, slyA, and any combination thereof. Exemplary attenuating mutations may be aroA, aroC, aroD, cdt, cya, crp, phoP, phoQ, ompR, galE, and htrA.

In certain embodiments, the above nucleic acid sequences may be placed under the control of a sugar regulated promoter wherein the sugar is present during in vitro growth of the recombinant bacterium, but substantially absent within an animal or human host. The cessation in transcription of the nucleic acid sequences listed above would then result in attenuation and the inability of the recombinant bacterium to induce disease symptoms.

In some embodiments, the recombinant bacterium comprises a deletion in an endogenous recF gene, which encodes the DNA replication and repair protein RecF. In some embodiments, the deletion of the endogenous recF gene is a partial deletion. In some embodiments, the deletion of the endogenous recF gene is a full-length deletion. In some embodiments, the endogenous recF gene is genetically altered to insert a transcriptional terminator in the open reading frame of the gene.

In some embodiments, the recombinant bacterium comprises a deletion in an endogenous recJ gene, which encodes the exonuclease RecJ. In some embodiments, the deletion of the endogenous recJ gene is a partial deletion. In some embodiments, the deletion of the endogenous recJ gene is a full-length deletion. In some embodiments, the endogenous recJ gene is genetically altered to insert a transcriptional terminator in the open reading frame of the gene.

In some embodiments, the recombinant bacterium comprises a deletion in an endogenous asd gene. As used herein, the terms asd and asdA refer to the same gene and are used interchangeably. In some embodiments, the deletion of the endogenous asd gene is a partial deletion. In some embodiments, the deletion of the endogenous asd gene is a full-length deletion. In some embodiments, the endogenous asdgene is genetically altered to insert a transcriptional terminator in the open reading frame of the gene. In some embodiments, the promoter of a endogenous asd gene is altered to include one or more regulatory elements (e.g., a sugar-responsive promoter). In one example, the bacterium is modified by using a ΔasdA mutation to eliminate the bacterium's ability to produce β-aspartate semialdehyde dehydrogenase, an enzyme essential for the synthesis of DAP. Other mutations that result in the abolition of the synthesis of DAP include, but are not limited to, dapA, dapB, dapC, dapD, dapE, dapF, and asd (see, e.g., U.S. Pat. No. 6,872,547, incorporated herein by reference). Other modifications that may be employed include modifications to a bacterium's ability to synthesize D-alanine or to synthesize D-glutamic acid (e.g., Δmurl mutations), which are both unique constituents of the peptidoglycan layer of the bacterial cell wall.

In some embodiments, the recombinant bacterium comprises an insertion of a c2 gene operably linked to an arabinose regulatable promoter. In some embodiments, the arabinose regulatable promoter is araC P_{araBAD} promoter, which is described in U.S. Pat. No. 10,988,729, incorporated herein by reference. Various embodiments may comprise the araC P_{araBAD} c2 gene cassette inserted into the asd nucleic acid sequence that encodes aspartate semialdehyde dehydrogenase. Since the araC nucleic acid sequence is transcribed in a direction that could lead to interference in the expression of adjacent nucleic acid sequences and adversely affect vaccine strain performance, a transcription termination (TT) sequence is generally inserted 3′ to the araC nucleic acid sequence. The chromosomal asd nucleic acid sequence is typically inactivated to enable use of plasmid vectors encoding the wild-type asd nucleic acid sequence in the balanced lethal host-vector system. This allows for stable maintenance of plasmids in vivo in the absence of any drug resistance attributes that are not permissible in live bacterial vaccines. In some of these embodiments, the wild-type asd nucleic acid sequence may be encoded by the vector described herein. The vector enables the regulated expression of an antigen encoding sequence through the repressible promoter.

In some embodiments, the recombinant bacterium comprises an asd gene (asd+). In some embodiments, the endogenous asd gene is not deleted in the bacterium. In some other embodiments, a heterologous wild-type asd gene is added to the bacterium (e.g., added to pAPEC-1 plasmid that was transferred into the bacterium). In some embodiments, the asd gene comprises a nucleic acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 12. In one embodiment, the asd gene has a nucleic acid sequence comprising SEQ ID NO: 12. In another embodiment, the asdgene has a nucleic acid sequence that consists of SEQ ID NO: 12. The presence of the asd gene enables the recombinant bacterium to grow without diaminopimelic acid (DAP). The nucleic acid sequence of an exemplary Salmonella asd gene is provided below:

(SEQ ID NO: 12)
atgaaaaatgttggttttatcggctggcgcggagtggtcggctctgttc
tcatgcaacgcatggtagaggagcgcgatttcgacgctattcgccctgt
tttcttttctacctcccagtttggacaggcggcgcccaccttcggcgac
acctccggcacgctacaggacgcttttgacctggatgcgctaaaagcgc
tcgatatcatcgtgacctgccagggcggcgattataccaacgaaattta
tccaaagctgcgcgaaagcggatggcagggttactggattgatgcggct
tctacgctgcgcatgaaagatgatgccattattattctcgacccggtca
accaggacgtgattaccgacggcctgaacaatggcgtgaagacctttgt
gggcggtaactgtaccgttagcctgatgttgatgtcgctgggcggtctc
tttgcccataatctcgttgactgggtatccgtcgcgacctatcaggccg
cctccggcggcggcgcgcgccatatgcgcgagctgttaacccagatggg
tcagttgtatggccatgtcgccgatgaactggcgacgccgtcttccgca
attcttgatattgaacgcaaagttacggcattgacccgcagcggcgagc
tgccggttgataactttggcgtaccgctggcgggaagcctgatcccctg
gatcgacaaacagctcgataacggccagagccgcgaagagtggaaaggc
caggcggaaaccaacaagattctcaatactgcctctgtgattccggttg
atggtttgtgtgtgcgcgtcggcgcgctgcgctgtcacagccaggcgtt
caccatcaagctgaaaaaagaggtatccattccgacggtggaagaactg
ctggcggcacataatccgtgggcgaaagtggtgccgaacgatcgtgata
tcactatgcgcgaattaaccccggcggcggtgaccggcacgttgactac
gccggttggtcgtctgcgtaagctgaacatggggccagagttcttgtcg
gcgtttaccgtaggcgaccagttgttatggggcgccgccgagccgctgc
gtcgaatgctgcgccagttggcctag

In some embodiments, the recombinant bacterium comprises a nucleic acid (e.g., a gene) that is operably linked to a repressor-regulatable promoter to facilitate the regulatable expression of the gene. Thus, in some embodiments, the recombinant bacterium comprises a nucleic acid comprising a gene encoding a repressor. In some embodiments, the gene encoding the repressor is operably-linked to a regulatable promoter. Methods of chromosomally integrating a nucleic acid sequence encoding a repressor operably-linked to a regulatable promoter are known in the art and detailed in the examples. In some embodiments, the nucleic acid sequence encoding a repressor is not integrated into a chromosomal locus such that the ability of the bacterium to colonize a host cell is disrupted. In some embodiments, the recombinant bacterium comprises a nucleic acid encoding a repressor that is integrated into the relA locus of the bacterial chromosome. In some embodiments, the recombinant bacterium comprises a nucleic acid encoding a repressor that is integrated into the endA locus of the bacterial chromosome. In some embodiments, the recombinant bacterium comprises at least one nucleic acid sequence encoding a repressor. In some embodiments, the recombinant bacterium comprises at least two, at least three, at least four, at least five, at least six or more nucleic acids encoding a repressor. In some embodiments, the nucleic acid encoding the repressor is present on a plasmid in the bacterium. In some embodiments, the nucleic acid encoding the repressor is located in the bacterial chromosome. If there is more than one nucleic acid sequence encoding a repressor, each nucleic acid sequence encoding a repressor may be operably linked to a regulatable promoter, such that each promoter is regulated by the same compound or condition. Alternatively, each nucleic acid sequence encoding a repressor may be operably linked to a regulatable promoter, each of which is regulated by a different compound or condition.

As used herein, a “repressor” refers to a biomolecule that represses the transcriptional activity of a promoter. In some embodiments, the repressor is synthesized by the recombinant bacterium in high enough quantities during in vitro culture, such that the transcription of a nucleic acid that is operably linked to a repressor-regulatable promoter is repressed. This may be particularly advantageous if, for example, expression of the product encoded by said nucleic acid impedes the in vitro growth of the bacterium, and/or the ability of the bacterium to infect and/or colonize a subject. In some embodiments, the nucleic acid that is operably-linked to the repressor-regulatable promoter expresses an antigen of interest. In some embodiments, the concentration of the repressor within the cell gradually decreases with each cell division cycle after transcription of the gene encoding the repressor decreases or ceases (e.g., in vivo). The use of a particular repressor, as described herein, may depend, in part, on the species, subspecies, strain or serovar of the recombinant bacterium being used. In some embodiments, the repressor is derived from the same species (e.g., the same bacterial species or the same phage) from which the repressor-regulatable promoter is derived. In some embodiments the repressor is not derived from the same bacterial species as the bacterial species in which the repressor is expressed. For example, in some embodiments, the repressor is derived from E. coli if the recombinant bacterium is of the genus Salmonella. Other suitable repressors include repressors derived from a bacteriophage.

A nucleic acid sequence encoding a repressor and regulatable promoter detailed above may be modified so as to optimize the expression level of the nucleic acid sequence encoding the repressor. The optimal level of expression of the nucleic acid sequence encoding the repressor may be estimated, or may be determined by experimentation. Such a determination should take into consideration whether the repressor acts as a monomer, dimer, trimer, tetramer, or higher multiple, and should also take into consideration the copy number of the vector encoding the antigen of interest. In an exemplary embodiment, the level of expression is optimized so that the repressor is synthesized while in a permissive environment (i.e., in vitro growth) at a level that substantially inhibits the expression of the nucleic acid encoding an antigen of interest, and is substantially not synthesized in a non-permissive environment, thereby allowing expression of the nucleic acid encoding an antigen of interest.

In some embodiments, the recombinant bacterium described herein is modified to comprise a nucleic acid comprising a lacl gene, which encodes the Lacl repressor protein. The expression of the lacl-encoded repressor in the recombinant bacterium described herein may be used to regulate the expression of a gene encoding an antigen of interest expressed by the bacterium. For example, in some embodiments, the expression of the lacl gene is regulated by a sugar-regulatable promoter (e.g., an arabinose-regulatable promoter). When cultured in the presence of arabinose, the recombinant bacterium will-synthesize the Lacl repressor protein, which in turn will repress the expression of a gene encoding an antigen of interest that is operably-linked to a Lacl-responsive promoter (e.g., P_{trc}, P_{iac}, P_{T7lac} and Ptac). Upon administration to the subject and in the absence of a source of arabinose, the synthesis of Lacl repressor ceases, leading to de-repression of the Lacl-responsive promoter and the subsequence causing expression of the antigen of interest. The concentration of Lacl in the cell decreases by about half at each cell division in vivo, leading to a gradual decreased level of repression and gradual increased synthesis of the antigen of interest.

In some embodiments, the nucleic acid comprising a lacl gene is located on a plasmid in the bacterium. In some embodiments, the nucleic acid comprising a lacl gene is located on a chromosome of the bacterium. In some embodiments, the nucleic acid comprising a lacl gene is located at the chromosomal locus corresponding to the locus of an endogenous relA gene that has been deleted or altered in the bacterial chromosome. In some embodiments, the recombinant bacterium is modified to comprise a nucleic acid comprising a lacl gene, whereby an endogenous copy of the lacl gene in the bacterial chromosome has been altered and/or deleted.

In some embodiments, the nucleic acid comprises an Escherichia coli lacl gene. The nucleic acid sequence of the E. coli lacl gene is provided below:

(SEQ ID NO: 9)
gtgaaaccagtaacgttatacgatgtcgcagagtatgccggtgtctctt
atcagaccgtttcccgcgtggtgaaccaggccagccacgtttctgcgaa
aacgcgggaaaaagtggaagcggcgatggcggagctgaattacattccc
aaccgcgtggcacaacaactgggggcaaacagtcgttgctgattggcgt
tgccacctccagtctggccctgcacgcgccgtcgcaaattgtcgcggcg
attaaatctcgcgccgatcaactgggtgccagcgtggtggtgtcgatgg
tagaacgaagcggcgtcgaagcctgtaaagcggcggtgcacaatcttct
cgcgcaacgcgtcagtgggctgatcattaactatccgctggatgaccag
gatgccattgctgtggaagctgcctgcactaatgttccggcgttatttc
ttgatgtctctgaccagacacccatcaacagtattattttctcccatga
agacggtacgcgactgggcgtggagcatctggtcgcattgggtcaccag
caaatcgcgctgttagcgggcccattaagttctgtctcggcgcgtctgc
gtctggctggctggcataaatatctcactcgcaatcaaattcagccgat
agcggaacgggaaggcgactggagtgccatgtccggttttcaacaaacc
atgcaaatgctgaatgagggcatcgttcccactgcgatgctggttgcca
acgatcagatggcgctgggcgcaatgcgcgccattaccgagtccgggct
gcgcgttggtgcggatatctcggtagtgggatacgacgataccgaagac
agctcatgttatatcccgccgttaaccaccatcaaacaggattttcgcc
tgctggggcaaaccagcgtggaccgcttgctgcaactctctcagggcca
ggcggtgaagggcaatcagctgttgcccgtctcactggtgaaaagaaaa
accaccctggcgcccaatacgcaaaccgcctctccccgcgcgttggccg
attcattaatgcagctggcacgacaggtttcccgactggaaagcgggca
gtga.

The amino acid sequence of the E. coli Lacl protein encoded by the nucleic acid of SEQ ID NO: 9 is provided below:

(SEQ ID NO: 10)
MKPVTLYDVAEYAGVSYQTVSRVVNQASHVSAKTREKVEAAMAELNYIP
NRVAQQLAGKQSLLIGVATSSLALHAPSQIVAAIKSRADQLGASVVVSM
VERSGVEACKAAVHNLLAQRVSGLIINYPLDDQDAIAVEAACTNVPALF
LDVSDQTPINSIIFSHEDGTRLGVEHLVALGHQQIALLAGPLSSVSARL
RLAGWHKYLTRNQIQPIAEREGDWSAMSGFQQTMQMLNEGIVPTAMLVA
NDQMALGAMRAITESGLRVGADISVVGYDDTEDSSCYIPPLTTIKQDFR
LLGQTSVDRLLQLSQGQAVKGNQLLPVSLVKRKTTLAPNTQTASPRALA
DSLMQLARQVSRLESGQ.

In some embodiments, the nucleic acid comprises a lacl gene, wherein the lacl gene comprises a nucleic acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 9. In some embodiments, the nucleic acid comprises a lacl gene, wherein the lacl gene comprises a nucleic acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to the nucleic acid sequence of SEQ ID NO: 9.

In some embodiments, the nucleic acid comprises a nucleic acid sequence encoding a Lacl protein, wherein said Lacl protein comprises an amino acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO: 10. In some embodiments, the nucleic acid comprises a nucleic acid sequence encoding a Lacl protein, wherein said Lacl protein comprises an amino acid sequence that is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homologous to the nucleic acid sequence of SEQ ID NO: 10.

In some embodiments, the nucleic acid comprises a lacl gene that is operably-linked to a regulatable promoter (e.g., a sugar-regulatable promoter). In some embodiments, the nucleic acid comprises a lacl gene that is operably-linked to a sugar-regulatable promoter. In some embodiments, the sugar regulatable promoter exhibits increased activity (e.g., increased transcription) in the presence of a specific sugar and decreased activity in the absence of a sugar. In some embodiments, the nucleic acid comprises a lacl gene that is operably-linked to a rhamnose-regulatable promoter (e.g., a sugar-regulatable promoter). In some embodiments, the nucleic acid comprises a lacl gene that is operably-linked to an arabinose-regulatable promoter. In some embodiments, the arabinose-regulatable promoter is P_{araBAD}. In some embodiments, the recombinant bacterium comprises the mutation ΔrelA::araC P_{araBAD} lacl TT.

The recombinant Salmonella bacterium of the present disclosure may be derived from any recombinant attenuated Salmonella bacterium, such as any of the bacterium disclosed in U.S. Pat. No. 10,988,729, incorporated herein by reference in its entirety. The recombinant Salmonella bacterium of the present disclosure will contains all of the genetic features or modifications of the bacterium from which it is derived that have not been altered.

In some embodiments, the recombinant Salmonella bacterium of the present disclosure comprises, or is derived from a Salmonella bacterium comprising: a araC P_{araBAD}-regulated murA gene (ΔPₘᵤᵣₐ::TT araC P_{araBAD} murA deletion-insertion mutation); a deletion-insertion mutation that inactivates the expression of asdA gene and inserts a c2 gene (ΔasdA::TT araC P_{araBAD} c2 deletion-insertion mutation); a deletion mutation in the gmd and a deletion in the fcl genes (Δ(gmd-fc)); and a deletion-insertion mutation that inactivates the expression of a relA gene and inserts a lacl gene (ΔrelA::araC P_{araBAD} lacl TT deletion-insertion mutation).

In some other embodiments, the recombinant Salmonella bacterium of the present disclosure comprises, or is derived from a Salmonella bacterium comprising: an araC P_{araBAD}-regulated murA gene (ΔPₘᵤᵣₐ::TT araC P_{araBAD} murA deletion-insertion mutation); a deletion-insertion mutation that inactivates the expression of asdA gene and inserts a c2 gene (ΔasdA::TT araC P_{araBAD} c2 deletion-insertion mutation); a deletion mutation of the gene cluster wza-wcaM (Δ(wza-wcaM)); a deletion-insertion mutation that inactivates the expression of a relA gene and inserts a lacl gene (ΔrelA::araC P_{araBAD} lacl TT deletion-insertion mutation); a deletion mutation in the recF gene (ΔrecF); a deletion mutation in the sifA gene (ΔsifA); a deletion mutation in the waaL gene (ΔwaaL46); and a deletion in a pagL gene and an insertion of a rhaRS P_{rhaBAD}-regulated waaL gene (ΔpagL::TT rhaRS P_{rhaBAD} waaL).

In some embodiments, the recombinant Salmonella bacterium comprises the genetic features of any of the strains listed in Table A below:

TABLE A
Non-limiting examples of recombinant Salmonella bacterium

| Strains (serotype)/ | |
|---|---|
| Plasmids | Relevant genotype and description |
| S. Typhimurium | |
| _{χ}11534 (O4) | ΔP_{murA25}::TT araC P_{araBAD} murA ΔasdA27::TT araC P_{araBAD} c2 Δ(gmd-fcl)-26 |
| | Δpmi-2426 ΔrelA198::araC P_{araBAD} lacI TT |
| _{χ}12341 (O4) | ΔP_{murA25}::TT araC P_{araBAD} murA ΔasdA27::TT araC P_{araBAD} c2 Δ(wza-wcaM)- |
| | 8 Δpmi-2426 ΔrelA197::araC P_{araBAD} lacI TT ΔrecF126 ΔsifA26 ΔwaaL46 |
| | ΔpagL64::TT rhaRS P_{rhaBAD} waaL |
| CS36 (O4) | ΔP_{murA25}::TT araC P_{araBAD} murA ΔasdA27::TT araC P_{araBAD} c2 Δ(wza-wcaM)- |
| | 8 Δpmi-2426 ΔrelA197::araC P_{araBAD} lacI TT ΔrecF126 ΔsifA26 ΔwaaL46 |
| | ΔpagL64::TT rhaRS P_{rhaBAD} waaL Δlrp12 |
| CS38 (O4) | ΔP_{murA25}::TT araC P_{araBAD} murA ΔasdA27::TT araC P_{araBAD} c2 Δ(gmd-fcl)-26 |
| | Δpmi-2426 ΔrelA198::araC P_{araBAD} lacI TT Δlrp12 |
| CS75 (O4) | ΔP_{murA25}::TT araC P_{araBAD} murA ΔasdA27::TT araC P_{araBAD} c2 Δ(wza-wcaM)- |
| | 8 ΔrelA197::araC P_{araBAD} lacI TT ΔrecF126 ΔsifA26 ΔwaaL46 pagL64::TT |
| | rhaRS P_{rhaBAD} waaL Δlrp12 (pmi+) |
| CS76 (O4) | ΔP_{murA25}::TT araC P_{araBAD} murA ΔasdA27::TT araC P_{araBAD} c2 Δ(gmd-fcl)-26 |
| | ΔrelA198::araC P_{araBAD} lacI TT Δlrp12 (pmi+) |
| CS77 (O78) | ΔP_{murA25}::TT araC P_{araBAD} murA ΔasdA27::TT araC P_{araBAD} c2 Δ(gmd-fcl)-26 |
| | ΔrelA198::araC P_{araBAD} lacI TT Δlrp12 Δ(rfbB-rfbP)::eco78ABCDEFGHIJK |
| | (pmi+) |
| CS90 (O78) | ΔP_{murA25}::TT araC P_{araBAD} murA ΔasdA27::TT araC P_{araBAD} c2 Δ(wza-wcaM)- |
| | 8 ΔrelA197::araC P_{araBAD} lacI TT ΔrecF126 ΔsifA26 ΔwaaL46 pagL64::TT |
| | rhaRS P_{rhaBAD} waaL Δlrp12 Δ(rfbB-rfbP)::eco78ABCDEFGHIJK (pmi+) |
| Curtiss 272 (O4) | ΔP_{murA25}::TT araC P_{araBAD} murA ΔasdA27::TT araC P_{araBAD} c2 Δ(gmd-fcl)-26 |
| | Δpmi-2426 ΔrelA198::araC P_{araBAD} lacI TT Δlrp12 |
| | pAPEC-1-Δtra::asd, pAPEC-2::ΔAMR, pAPEC-3 |
| Curtiss 273 (O4) | ΔP_{murA25}::TT araC P_{araBAD} murA ΔasdA27::TT araC P_{araBAD} c2 Δ(wza-wcaM)- |
| | 8 Δpmi-2426 ΔrelA197::araC P_{araBAD} lacI TT ΔrecF126 ΔsifA26 ΔwaaL46 |
| | ΔpagL64::TT rhaRS P_{rhaBAD} waaL Δlrp12 |
| | pAPEC-1-Δtra::asd, pAPEC-2::ΔAMR, pAPEC-3 |
| Curtiss 274 (O78) | ΔP_{murA25}::TT araC P_{araBAD} murA ΔasdA27::TT araC P_{araBAD} c2 Δ(gmd-fcl)-26 |
| | Δpmi-2426 ΔrelA198::araC P_{araBAD} lacI TT Δlrp12 Δ(rfbB- |
| | rfbP)::eco78ABCDEFGHIJK |
| | pAPEC-1-Δtra::asd, pAPEC-2::ΔAMR, pAPEC-3 |
| Curtiss 275 (O78) | ΔP_{murA25}::TT araC P_{araBAD} murA ΔasdA27::TT araC P_{araBAD} c2 Δ(wza-wcaM)- |
| | 8 Δpmi-2426 ΔrelA197::araC P_{araBAD} lacI TT ΔrecF126 ΔsifA26 ΔwaaL46 |
| | ΔpagL64::TT rhaRS P_{rhaBAD} waaL Δlrp12 Δ(rfbB-rfbP)::eco78ABCDEFGHIJK |
| | pAPEC-1-Δtra::asd, pAPEC-2::ΔAMR, pAPEC-3 |
| Curtiss 276 (O78) | ΔP_{murA25}::TT araC P_{araBAD} murA ΔasdA27::TT araC P_{araBAD} c2 Δ(gmd-fcl)-26 |
| | ΔrelA198::araC P_{araBAD} lacI TT Δlrp12 Δ(rfbB-rfbP)::eco78ABCDEFGHIJK |
| | (pmi+) |
| | pAPEC-1-Δtra::asd, pAPEC-2::ΔAMR, pAPEC-3 |
| Curtiss 277 (O78) | ΔP_{murA25}::TT araC P_{araBAD} murA ΔasdA27::TT araC P_{araBAD} c2 Δ(wza-wcaM)- |
| | 8 ΔrelA197::araC P_{araBAD} lacI TT ΔrecF126 ΔsifA26 ΔwaaL46 pagL64::TT |
| | rhaRS P_{rhaBAD} waaL Δlrp12 Δ(rfbB-rfbP)::eco78ABCDEFGHIJK (pmi+) |
| | pAPEC-1-Δtra::asd, pAPEC-2::ΔAMR, pAPEC-3 |
| Curtiss 280 (O78) | ΔP_{murA25}::TT araC P_{araBAD} murA ΔasdA27::TT araC P_{araBAD} c2 Δ(gmd-fcl)-26 |
| | ΔrelA198::araC P_{araBAD} lacI TT Δlrp12 Δ(rfbB-rfbP)::eco78ABCDEFGHIJK |
| | (pmi+) |
| | pCHC116 |
| Curtiss 281 (O78) | ΔP_{murA25}::TT araC P_{araBAD} murA ΔasdA27::TT araC P_{araBAD} c2 Δ(wza-wcaM)- |
| | 8 ΔrelA197::araC P_{araBAD} lacI TT ΔrecF126 ΔsifA26 ΔwaaL46 pagL64::TT |
| | rhaRS P_{rhaBAD} waaL Δlrp12 Δ(rfbB-rfbP)::eco78ABCDEFGHIJK (pmi+) |
| | pCHC116 |

### Pharmaceutical Compositions and Methods of Use

A recombinant bacterium provided in the present disclosure may be administered to a subject as a pharmaceutical composition. In some embodiments, the pharmaceutical composition may be used as a vaccine to elicit an immune response to an APEC. In an exemplary embodiment, the immune response is protective.

Pharmaceutical compositions may be administered to any host capable of mounting an immune response. Such hosts may include all vertebrates, for example, mammals, including domestic animals, agricultural animals, laboratory animals, and humans, and various species of birds, including domestic birds and birds of agricultural importance. Preferably, the host is a warm-blooded animal. In one embodiment, the host is a cow. In some embodiments, the host is an equine. In another embodiment, the host is an avian. In another embodiment, the host is a human. The pharmaceutical composition can be administered to the subject as a prophylactic or for treatment purposes.

In some embodiments, the recombinant bacterium is alive when administered to a host in a pharmaceutical composition described herein. Suitable vaccine composition formulations and methods of administration are detailed below.

A pharmaceutical composition comprising a recombinant bacterium may optionally comprise one or more possible additives, such as carriers, preservatives, stabilizers, adjuvants, and other substances.

In one embodiment, the pharmaceutical composition comprises an adjuvant. Adjuvants are optionally added to increase the ability of the vaccine to trigger, enhance, or prolong an immune response. In exemplary embodiments, the use of a live attenuated recombinant bacterium may act as a natural adjuvant. In some embodiments, the recombinant bacterium synthesizes and secretes an immune modulator. Additional materials, such as cytokines, chemokines, and bacterial nucleic acid sequences naturally found in bacteria, like CpG, are also potential vaccine adjuvants.

In some embodiments, the pharmaceutical composition comprises buffered saline (e.g., phosphate-buffered saline (PBS)).

In some embodiments, the pharmaceutical composition comprises a food product.

In another embodiment, the pharmaceutical may comprise a pharmaceutical carrier (or excipient). Such a carrier may be any solvent or solid material for encapsulation that is non-toxic to the inoculated host and compatible with the recombinant bacterium. A carrier may give form or consistency, or act as a diluent. Suitable pharmaceutical carriers may include liquid carriers, such as normal saline and other non-toxic salts at or near physiological concentrations, and solid carriers not used for humans, such as talc or sucrose, or animal feed. Carriers may also include stabilizing agents, wetting and emulsifying agents, salts for varying osmolarity, encapsulating agents, buffers, and skin penetration enhancers. Carriers and excipients as well as formulations for parenteral and nonparenteral drug delivery are set forth in Remington's Pharmaceutical Sciences 19th Ed. Mack Publishing (1995). When used for administering via the bronchial tubes, the pharmaceutical composition is preferably presented in the form of an aerosol.

In some embodiments, the pharmaceutical composition is delivered to a farm animal (e.g., poultry). In some embodiments, the pharmaceutical composition is delivered as a course spray (e.g., for use in hatcheries for delivery to poultry). In some embodiments, the pharmaceutical composition is delivered through oral immunization, such as in drinking water or mixed with food.

Care should be taken when using additives so that the live recombinant bacterium is not killed, or have its ability to effectively colonize lymphoid tissues such as the gut-associated lymphoid tissue (GALT), nasal-associated lymphoid tissue (NALT) and bronchus-associated lymphoid tissue (BALT) compromised by the use of additives. Stabilizers, such as lactose or monosodium glutamate (MSG), may be added to stabilize the pharmaceutical composition against a variety of conditions, such as temperature variations or a freeze-drying process. The recombinant bacterium may also be co-administered with glutamate and/or arginine as described herein.

The dosages of a pharmaceutical composition can and will vary depending on the recombinant bacterium, the regulated antigen, and the intended host, as will be appreciated by one of skill in the art. Generally speaking, the dosage need only be sufficient to elicit a protective immune response in a majority of hosts. Routine experimentation may readily establish the required dosage. Typical initial dosages of vaccine for oral administration could be about 1×10⁷ to 1×10¹⁰ CFU depending upon the age of the host to be immunized. Administering multiple dosages may also be used as needed to provide the desired level of protective immunity.

In order to stimulate a preferred response of the GALT, NALT or BALT cells, administration of the pharmaceutical composition directly into the gut, nasopharynx, or bronchus is preferred, such as by oral administration, intranasal administration, gastric intubation or in the form of aerosols, although other methods of administering the recombinant bacterium, such as intravenous, intramuscular, subcutaneous injection or intramammary, intrapenial, intrarectal, vaginal administration, or other parenteral routes, are possible, e.g., for anti-cancer applications.

In some embodiments, these compositions are formulated for administration by injection (e.g., intraperitoneally, intravenously, subcutaneously, intradermally, intramuscularly, etc.).

In an aspect, the disclosure provides a method for eliciting an immune response against an avian pathogenic E. coli in a subject. In another aspect, the disclosure provides a method for vaccinating a subject against an avian pathogenic E. coli. The method comprises administering to the subject an effective amount of a pharmaceutical composition comprising a recombinant bacterium described herein.

In a further embodiment, a recombinant bacterium described herein may be used in a method for ameliorating one or more symptoms of an infectious disease in a host in need thereof. The method comprises administering an effective amount of a pharmaceutical composition comprising a recombinant bacterium as described herein.

In some embodiments, the recombinant bacterium described herein is used to induce an immune response in poultry (e.g., as a vaccine). When used in poultry (e.g., chicken, turkey, goose, or duck), the recombinant bacterium may be administered by course spray and thereby inoculate the conjunctiva-associated lymphoid tissue (CALT) via eye exposure, the nasal-associated lymphoid tissue (NALT) and bronchus-associated lymphoid tissue (BALT) via respiratory exposure and the gut-associated lymphoid tissue (GALT) via oral exposure. In some embodiments, the recombinant bacterium described herein is administered to newly-hatched chicks.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure.

### Example 1: Background

Pathogenic Escherichia coli isolates have been classified as either intestinal pathogenic E. coli (IPEC) or extraintestinal pathogenic E. coli (ExPEC) based on the anamnestic clinical reports and virulence features [Russo and Johnson, 2000; Russo and Johnson, 2003]. Extraintestinal pathogenic E. coli (ExPEC) pathotype includes newborn avian pathogenic E. coli (APEC), meningitis E. coli (NMEC), uropathogenic E. coli (UPEC), and septicemic E. coli (SEPEC). [Russo and Johnson, 2000]. APEC causes diverse systemic infections in poultry including chickens, turkeys, ducks, and many other avian species [Barnes et al, 2003; Dho-Moulin and Fairbrother, 1999]. The most common diseases caused by APEC in chickens collectively known as colibacillosis infections including airsacculitis, cellulitis, coligranuloma, egg peritonitis, omphalitis, osteomyelitis/arthritis, perihepatitis, pericarditis, and salphingitis [Dziva and Stevens, 2008]. Colibacillosis caused by APEC is a devastating disease of poultry that results in severe economic losses annually to the poultry industry worldwide [Newman et al., 2021]. Multiple APEC serotypes have been associated with colibacillosis cases in the field outbreaks; however, three serotypes (O78, O2, and O1) account for the majority (more than 80%) of the cases [Dho-Moulin and Fairbrother, 1999; Ghunaim et al, 2014]. The pathogenicity of APEC is still poorly understood. The most prevalent virulence traits associated with APEC are adhesins (type 1 and P fimbriae, Temperature-sensitive hemagglutinin Tsh, and curli), capsules, iron acquisition systems, and serum resistance [Dho-Moulin and Fairbrother, 1999]. Large plasmids are commonly found in APEC strains and are associated with the virulence of APEC [Dozois et al, 2000; Doetkott et al, 1996; Rodriguez-Siek et al, 2005]. Their importance has been demonstrated by the occurrence of plasmid-encoded genes shared among APEC strains [Dozois et al, 2003] and the presence of virulence genes in plasmids [Johnson et al, 2002; Stehling et al, 2003]. Most virulence genes associated with APEC are often located on IncF plasmids, named CoIV plasmids because their ability to code for production of colicin V, a small protein from the microcine family [Johnson et al, 2002; Johnson et al, 2004; Tivendale et al, 2004]. CoIV plasmids are associated with E. coli in general and with APEC in particular. Their link with the ability of bacteria to cause disease in production animals has been demonstrated [Skyberg et al, 2006; Smith et al, 1980; Wooley et al, 1998]. In vivo expression of genes encoded by the CoIV plasmid, including Tsh, aerobactin, and the Iro system, has been confirmed by using SCOTS [Dozois et al, 2003] and the occurrence of the putative virulence genes of APEC on non-CoIV plasmids has also been shown [Dozois et al, 2003; Johnson et al, 2006; Dozois et al, 2000]. The complete nucleotide sequence analysis of the large CoIV/IncF virulence plasmid pAPEC-1 (103,275-bp) from the APEC strain X⁷¹²² (serogroup O78:K80:H9) revealed that a putative virulence region spanning an 80-kb region of pAPEC-1 comprises genes responsible for the potential virulence factors including four iron acquisition systems (iutA, iucABCD, sitABCD, and iroBCDN), temperature-sensitive hemagglutinin (tsh), a colicin V operon, increasing serum sensitivity (iss), ompT, hlyF, and etsABC [Mellata et al, 2009]. In addition, it has been reported that pAPEC-1-cured X⁷¹²² bacteria were attenuated in chickens, caused few lesions of pericarditis and perihepatitis, did not persist in the blood, and poorly colonized the lung, spleen, and liver [Dozois et al, 2000]. This APEC strain X⁷¹²² has two additional large plasmids (FIG. 1), pAPEC-2 (82,676-bp) and pAPEC-3 (56,676-bp), that also have virulence attributes [Mellata et al, 2010].

In the case of interactions between animal host and bacterial pathogen, the bacterial surface is the first line of contact with the host immune system. Among the bacterial surface molecules undergoing pathogenic interactions, LPS constitute the major structural component of the outer membrane of Gram-negative bacteria. The LPS fraction comprises about 10-15% of the total molecules in the outer membrane and is estimated to occupy about 75% of the bacterial surface [Lerouge and Vanderleyden, 2001]. LPS consists of three different components or regions: lipid A, core oligosaccharide, and O-polysaccharide. The structure of lipid A is highly conserved among Gram-negative bacteria. Among Enterobacteriaceae, lipid A is virtually constant. Lipid A is the lipid component of LPS that contains the hydrophobic, membrane-anchoring region of LPS. The core oligosaccharide regions consist of a short chain of sugars, which connects the lipid A anchor to O-antigen, and can be divided into inner and outer core regions [Osborn and Wu, 1980]. The O-antigen or O-side chain is attached to the core polysaccharide and extends from the core out into the environment. It consists of repeating oligosaccharide subunits made up of three to five sugars. The individual chains can vary in length ranging up to 40 repeat units. The O-polysaccharide is much longer than the core oligosaccharide and it contains the hydrophilic domain of the LPS. At least 20 different sugars are known to occur and many of these sugars are characteristically unique dideoxyhexoses such as abequose, colitose, paratose, and tyvelose, which are rarely found elsewhere in nature. The composition of sugars in the O-polysaccharide, especially terminal units, confers immunological specificity of the O-antigen and contributes to the wide variety of antigenic types (serotypes) between species and even strains of Gram-negative bacteria [Lerouge and Vanderleyden, 2001]. LPS O-antigen has been reported to be a potent antigen for inducing specific antibody response which is protective against E. coli pathogens in animal models [Pluschke and Achtman, 1985; Schief et al, 1993; Cryz Jr et al, 1995]. In addition, specific antibody against LPS O-antigen has been shown to be protective against other gram-negative pathogens [Zhang et al, 2009; Ding et al, 1990; Moustafa et al, 2023].

Live attenuated bacterial vaccines have been created by recombinant DNA technologies for nearly 4 decades [Lewis, 2007]. Over this time, a number of animal studies have shown that these live attenuated recombinant bacterial vaccines can induce protective immunity by delivering antigens expressed by the bacterium itself and DNA vaccines to be expressed in target eukaryotic cells. Recombinant attenuated Salmonella vaccines (RASVs) have been significantly improved to exhibit the same or better attributes than wild-type parental strains to colonize internal lymphoid tissues and persist there to serve as factories to continuously synthesize and deliver recombinant antigens which are selected to induce protective immunity to infection by that pathogen [Clark-Curtiss and Curtiss, 2018]. These RASVs are designed to attributes maximize their abilities to elicit mucosal and systemic antibody responses and cell-mediated immune responses. Additionally, RASVs can be administered by a convenient oral vaccination route which is a significant advantage in the vaccine industry.

In the present study, the RASV-based APEC vaccines are designed (FIG. 1) to produce the APEC virulence plasmid-encoded virulence factors and APEC O78-antigen to induce protective immunity against APEC infections.

### Example 2: Materials and Methods

### Bacterial Strains, Plasmids, Culture Conditions, and Reagents

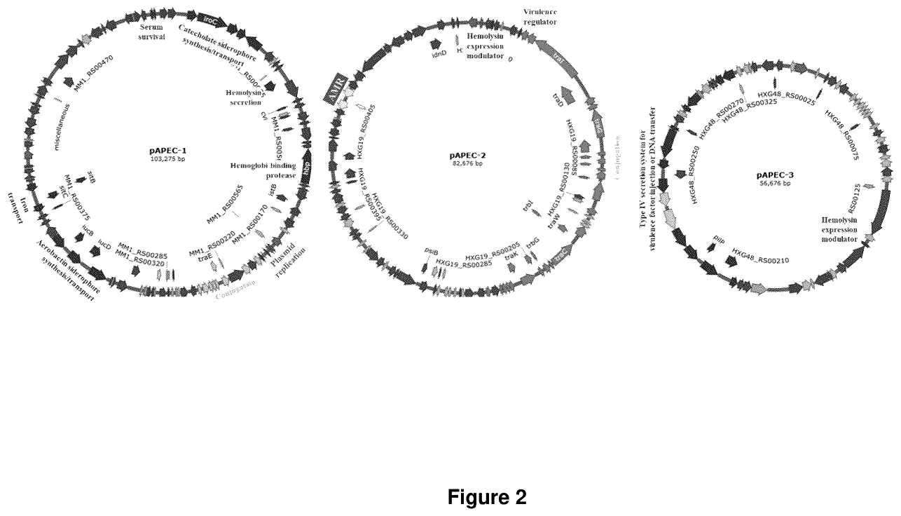

The bacterial strains and plasmids used in the present invention are listed in Table 1. The starting strains, X¹¹⁵³⁴ and X¹²³⁴¹, originated from the wild-type Salmonella enterica serovar Typhimurium strain X³⁷⁶¹ (UK-1). The construction history of X¹²³⁴¹-lineage strains including X¹¹⁵³⁴ and X¹²³⁴¹ is described in FIG. 2. The wild-type strain X³⁷⁶¹(UK-1) was recovered from the spleen of a chicken orally infected with a highly virulent serovar Typhimurium strain originally isolated from a horse [Curtiss 3rd et al, 1991]. This wild-type strain UK-1 is highly virulent in chicks and mice [Curtiss 3rd et al, 1991; Zhang et al, 1997]. Bacterial strains were grown in SBM broth or LB medium [Bertani, 1951] at 37° C. SBM broth is a modified LB medium containing 0.5% yeast extract, 2% tryptone, and 5% NaCl. Diaminopimelic acid (50 μg/ml) was added to the culture media for growing strains with Δasd mutations. Antibiotics were used as needed at the following concentrations: streptomycin (Sm), 20 or 100 μg/ml; chloramphenicol (Cm), 20 μg/ml, and tetracycline (Tc), 10 μg/ml. All antibiotics and chemicals were purchased from MilliporeSigma or Thermo Fisher Scientific. Anti-O4 rabbit antibody was purchased from Abcam. Goat anti-rabbit IgG-alkaline phosphatase antibody was purchased from Thermo Fisher Scientific.

TABLE 1
Bacterial strains and plasmids

| Strains (serotype)/ | | Source or |
|---|---|---|
| Plasmids | Relevant genotype and description | reference |
| Strains | | |
| E. coli | | |
| _{χ}7122 (O78) | Wild-type APEC isolate | |
| _{χ}7213 | thr-1 leuB6 fhuA21 lacY1 glnV44 recAl ΔasdA4 thi-1 RP4-2-Tc::Mu[λ- | Roland et |
| | pir]; Km^{R} | al, 1999 |
| S. Typhimurium | | |
| _{χ}3761 (O4) | Wild-type UK-1 strain | Curtiss 3^{rd} |
| | | et al, 1991 |
| _{χ}11534 (O4) | ΔP_{murA25}::TT araC P_{araBAD} murA ΔasdA27::TT araC P_{araBAD} c2 Δ(gmd- | This study |
| | fcl)-26 Δpmi-2426 ΔrelA198::araC P_{araBAD} lacI TT | |
| _{χ}12341 (O4) | ΔP_{murA25}::TT araC P_{araBAD} murA ΔasdA27::TT araC P_{araBAD} c2 Δ(wza- | Curtiss 3^{rd} |
| | wcaM)-8 Δpmi-2426 ΔrelA197::araC P_{araBAD} lacI TT ΔrecF126 | and Wang, |
| | ΔsifA26 ΔwaaL46 ΔpagL64::TT rhaRS P_{rhaBAD} waaL | 2018 |
| CS36 (O4) | ΔP_{murA25}::TT araC P_{araBAD} murA ΔasdA27::TT araC P_{araBAD} c2 Δ(wza- | This study |
| | wcaM)-8 Δpmi-2426 ΔrelA197::araC P_{araBAD} lacI TT ΔrecF126 | |
| | ΔsifA26 ΔwaaL46 ΔpagL64::TT rhaRS P_{rhaBAD} waaL Δlrp12 | |
| CS38 (O4) | ΔP_{murA25}::TT araC P_{araBAD} murA ΔasdA27::TT araC P_{araBAD} c2 Δ(gmd- | This study |
| | fcl)-26 Δpmi-2426 ΔrelA198::araC P_{araBAD} lacI TT Δlrp12 | |
| CS75 (O4) | ΔP_{murA25}::TT araC P_{araBAD} murA ΔasdA27::TT araC P_{araBAD} c2 Δ(wza- | This study |
| | wcaM)-8 ΔrelA197::araC P_{araBAD} lacI TT ΔrecF126 ΔsifA26 ΔwaaL46 | |
| | pagL64::TT rhaRS P_{rhaBAD} waaL Δlrp12 (pmi+) | |
| CS76 (O4) | ΔP_{murA25}::TT araC P_{araBAD} murA ΔasdA27::TT araC P_{araBAD} c2 Δ(gmd- | This study |
| | fcl)-26 ΔrelA198::araC P_{araBAD} lacI TT Δlrp12 (pmi+) | |
| CS77 (O78) | ΔP_{murA25}::TT araC P_{araBAD} murA ΔasdA27::TT araC P_{araBAD} c2 Δ(gmd- | This study |
| | fcl)-26 ΔrelA198::araC P_{araBAD} lacI TT Δlrp12 Δ(rfbB- | |
| | rfbP)::eco78ABCDEFGHIJK (pmi+) | |
| CS90 (O78) | ΔP_{murA25}::TT araC P_{araBAD} murA ΔasdA27::TT araC P_{araBAD} c2 Δ(wza- | This study |
| | wcaM)-8 ΔrelA197::araC P_{araBAD} lacI TT ΔrecF126 ΔsifA26 ΔwaaL46 | |
| | pagL64::TT rhaRS P_{rhaBAD} waaL Δlrp12 Δ(rfbB- | |
| | rfbP)::eco78ABCDEFGHIJK (pmi+) | |
| Curtiss 272 (O4) | ΔP_{murA25}::TT araC P_{araBAD} murA ΔasdA27::TT araC P_{araBAD} c2 Δ(gmd- | This study |
| | fcl)-26 Δpmi-2426 ΔrelA198::araC P_{araBAD} lacI TT Δlrp12 | |
| | pAPEC-1-Δtra::asd, pAPEC-2::ΔAMR, pAPEC-3 | |
| Curtiss 273 (O4) | ΔP_{murA25}::TT araC P_{araBAD} murA ΔasdA27::TT araC P_{araBAD} c2 Δ(wza- | This study |
| | wcaM)-8 Δpmi-2426 ΔrelA197::araC P_{araBAD} lacI TT ΔrecF126 | |
| | ΔsifA26 ΔwaaL46 ΔpagL64::TT rhaRS P_{rhaBAD} waaL Δlrp12 | |
| | pAPEC-1-Δtra::asd, pAPEC-2::ΔAMR, pAPEC-3 | |
| Curtiss 274 (O78) | ΔP_{murA25}::TT araC P_{araBAD} murA ΔasdA27::TT araC P_{araBAD} c2 Δ(gmd- | This study |
| | fcl)-26 Δpmi-2426 ΔrelA198::araC P_{araBAD} lacI TT Δlrp12 Δ(rfbB- | |
| | rfbP)::eco78ABCDEFGHIJK | |
| | pAPEC-1-Δtra::asd, pAPEC-2::ΔAMR, pAPEC-3 | |
| Curtiss 275 (O78) | ΔP_{murA25}::TT araC P_{araBAD} murA ΔasdA27::TT araC P_{araBAD} c2 Δ(wza- | This study |
| | wcaM)-8 Δpmi-2426 ΔrelA197::araC P_{araBAD} lacI TT ΔrecF126 | |
| | ΔsifA26 ΔwaaL46 ΔpagL64::TT rhaRS P_{rhaBAD} waaL Δlrp12 Δ(rfbB- | |
| | rfbP)::eco78ABCDEFGHIJK | |
| | pAPEC-1-Δtra::asd, pAPEC-2::ΔAMR, pAPEC-3 | |
| Curtiss 276 (O78) | ΔP_{murA25}::TT araC P_{araBAD} murA ΔasdA27::TT araC P_{araBAD} c2 Δ(gmd- | This study |
| | fcl)-26 ΔrelA198::araC P_{araBAD} lacI TT Δlrp12 Δ(rfbB- | |
| | rfbP)::eco78ABCDEFGHIJK (pmi+) | |
| | pAPEC-1-Δtra::asd, pAPEC-2::ΔAMR, pAPEC-3 | |
| Curtiss 277 (O78) | ΔP_{murA25}::TT araC P_{araBAD} murA ΔasdA27::TT araC P_{araBAD} c2 Δ(wza- | This study |
| | wcaM)-8 ΔrelA197::araC P_{araBAD} lacI TT ΔrecF126 ΔsifA26 ΔwaaL46 | |
| | pagL64::TT rhaRS P_{rhaBAD} waaL Δlrp12 Δ(rfbB- | |
| | rfbP)::eco78ABCDEFGHIJK (pmi+) | |
| | pAPEC-1-Δtra::asd, pAPEC-2::ΔAMR, pAPEC-3 | |
| Curtiss 280 (O78) | ΔP_{murA25}::TT araC P_{araBAD} murA ΔasdA27::TT araC P_{araBAD} c2 Δ(gmd- | This study |
| | fcl)-26 ΔrelA198::araC P_{araBAD} lacI TT Δlrp12 Δ(rfbB- | |
| | rfbP)::eco78ABCDEFGHIJK (pmi+) | |
| | pCHC116 (empty vector) | |
| Curtiss 281 (O78) | ΔP_{murA25}::TT araC P_{araBAD} murA ΔasdA27::TT araC P_{araBAD} c2 Δ(wza- | This study |
| | wcaM)-8 ΔrelA197::araC P_{araBAD} lacI TT ΔrecF126 ΔsifA26 ΔwaaL46 | |
| | pagL64::TT rhaRS P_{rhaBAD} waaL Δlrp12 Δ(rfbB- | |
| | rfbP)::eco78ABCDEFGHIJK (pmi+) | |
| | pCHC116 (empty vector) | |
| Plasmids | | |
| pAPEC-1 | APEC virulence plasmid 1 from _{χ}7122, 103,275 bp, IncFIB- | Mellata et |
| | incFIC/ColV | al, 2010 |
| pAPEC-2 | APEC virulence plasmid 2 from _{χ}7122, 82,676 bp, IncFII, Sm^{R} | Mellata et |
| | | al, 2010 |
| pAPEC-3 | APEC virulence plasmid 3 from _{χ}7122, 56,676 bp, IncI2 | Mellata et |
| | | al, 2010 |
| pAPEC1-Δtra::asd | pAPEC-1 derivative with Δ(traM-traX)::asd mutation, IncFIB- | This study |
| | incFIC/ColV, Δsd+ | |
| pΔPEc2-ΔAMR | pAPEC-2 derivative, deletion of the AMR genes, IncFII | This study |
| pCHC116 | pBBRI ori, low copy number cloning vector, Asd+ | This study |
| pCHSUI-1 | Suicide vector (R6K ori) for gene replacement, Tc^{R} | Baek et al, |
| | | 2007 |
| pCHSUI-2 | Suicide vector (R6K ori) for gene replacement, Cm^{R} | This study |
| pCHSUI-Δlrp | Suicide vector (R6K ori) for introducing Δlrp mutation, Tc^{R} | This study |
| pCHSUI-pmi | Suicide vector (R6K ori) for introducing wild-type pmi gene back, Tc^{R} | This study |
| pCHSUI-APEC-O78 | Suicide vector (R6K ori) for the serotype conversion (O4 to O78), Tc^{R} | This study |
| pCHSUI2-APEC- | Suicide vector (R6K ori) for introducing Δ(traM-traX)::asd mutation | This study |
| 1-Δtra::asd | pAPEC-1, Cm^{R} | |
| pCHSUI-APEC-2- | Suicide vector (R6K ori) for introducing ΔAMR mutation on pAPEC-2, | This study |
| ΔAMR | Cm^{R} | |
| pCHSUI-APEC-3 | Suicide vector (R6K ori) for the pAPEC-3 conjugal transfer and | This study |
| | selection marker, Tc^{R} | |

Sm^{R}, streptomycin resistance;
Cm^{R}, chloramphenicol resistance;
Tc^{R}, tetracycline resistance

### DNA Manipulations

Plasmid DNA and genomic DNA were isolated by using QIAprep Spin Miniprep Kit (Qiagen) and Genomic DNA Purification Kit (Thermo Fisher Scientific), respectively. Restriction enzymes and DNA-modifying enzymes were used as recommended by the manufacturers (New England Biolabs or Thermo Fisher Scientific). The primers used in the present study are listed in Table 2.

TABLE 2
Primers used in this study

| Primer | SEQ | |
|---|---|---|
| name | ID NO: | Sequence (5′ to 3′) |
| CHB250 | 13 | CGAATTCCTGCAGCCCGGGGGATCCACTAGTCGTCGACAAGCCAATGATTCAGTAC |
| CHB251 | 14 | ACCGCGGTGGCGGCCGCTCTAGAACTAGTGACATCCACCAGGTAGTTTCCGTCTTC |
| CHB433 | 15 | ACCGCGGTGGCGGCCGCTCTAGAACTAGTGCTGATAAAGCTCAGGATTCTG |
| CHB435 | 16 | CGAATTCCTGCAGCCCGGGGGATCCACTAGTCAGCTTAACGGTCTTCAGGTTC |
| CHB453 | 17 | CGAATTCCTGCAGCCCGGGGGATCCACTAGTGAAAACAGAACGTCGTCGGGATC |
| CHB454 | 18 | TAACACGGAACAGGTGCAAAATCGCTG |
| CHB455 | 19 | CAGCGATTTTGCACCTGTTCCGTGTTAGTCTCTCTGTATTCCTTCCCTACTC |
| CHB456 | 20 | ACCGCGGTGGCGGCCGCTCTAGAACTAGTTGTTTGGGCTATGACCGATGGCTAC |
| CHB535 | 21 | CGAATTCCTGCAGCCCGGGGGATCCACTAGTCTGTTTGCGGTATGCGGTGAAC |
| CHB536 | 22 | GTTCACTTCCAGAGCGGGCTGTTAC |
| CHB537 | 23 | GTAACAGCCCGCTCTGGAAGTGAACTTCATTGCTTACGGCCTTGAG |
| CHB538 | 24 | ACCGCGGTGGCGGCCGCTCTAGAACTAGTTTCGATGCCAGATGGGCATGTAC |
| CHB541 | 25 | CGAATTCCTGCAGCCCGGGGGATCCACTAGTAGTACCAGAAGCAACAGCATC |
| CHB542 | 26 | CTGGTATGATGCTGGCGCTGATTCTG |
| CHB543 | 27 | CAGAATCAGCGCCAGCATCATACCAGATTTAAATCAAAGCGGTGTCGGCGCGTTGTTGTAG |
| CHB544 | 28 | ACCGCGGTGGCGGCCGCTCTAGAACTAGTACCGGTATCTGACAATGTCTG |
| CHB546 | 29 | CGAATTCCTGCAGCCCGGGGGATCCACTAGTCTCAGGAGTTAAGATAAGGACGGTG |
| CHB547 | 30 | ACCGCGGTGGCGGCCGCTCTAGAACTAGTGGGAGAGGATTAATCATGATTGAC |
| CHB550 | 31 | ATCAGCGCCAGCATCATACCAGATTTGCCATCGTCCACATATCCAC |
| CHB551 | 32 | ACAACGCGCCGACACCGCTTTGATTTGCTTGAGTACTAGGCCAACTG |
| CHB559 | 33 | ATTTAAATGGTGCGTATTAAGCTTACCGAGAAGTACTG |
| CHB560 | 34 | TAAGCTTAATACGCACCATTTAAATCACCAACCATTTATCACGGTTG |
| CHB561 | 35 | CGGTAAGCTTAATACGCACCATTTGCCGATCTGTTGCTTCAGCATTG |
| CHB562 | 36 | CAACCGTGATAAATGGTTGGTGATTTTGAAAGCTTGATCGGATATGACGGCTG |

The lrp-deletion (Δlrp) mutation was introduced into the RASV strains by the gene replacement method [Kaniga et al, 1991] using a suicide vector pCHSUI-Δlrp. Two DNA fragments, Δlrp-L (1,286 bp, upstream region of lrp gene) and Δlrp-R (1,015 bp, downstream region of lrp gene) were amplified by PCR from the wild-type X³⁷⁶¹ using the primer set 1 (CHB455 and CHB456) and set 2 (CHB453 and CHB454), respectively. These two DNA fragments were cloned into the Spel site of the suicide vector pCHSUI-1 using GeneArt™ Seamless Cloning and Assembly Enzyme Mix (Thermo Fisher Scientific) to generate pCHSUI-Δlrp.

The pmi mutations in the RASV strains were replaced with the wild-type pmi gene using a suicide vector pCHSUI-pmi. A 3,224 bp DNA fragment containing the wild-type pmi gene was amplified by PCR from the wild-type X³⁷⁶¹ using the primers CHB433 and CHB435. This DNA fragment was cloned into the Spel site of the suicide vector pCHSUI-1 using GeneArt™ Seamless Cloning and Assembly Enzyme Mix (Thermo Fisher Scientific) to generate pCHSUI-pmi.

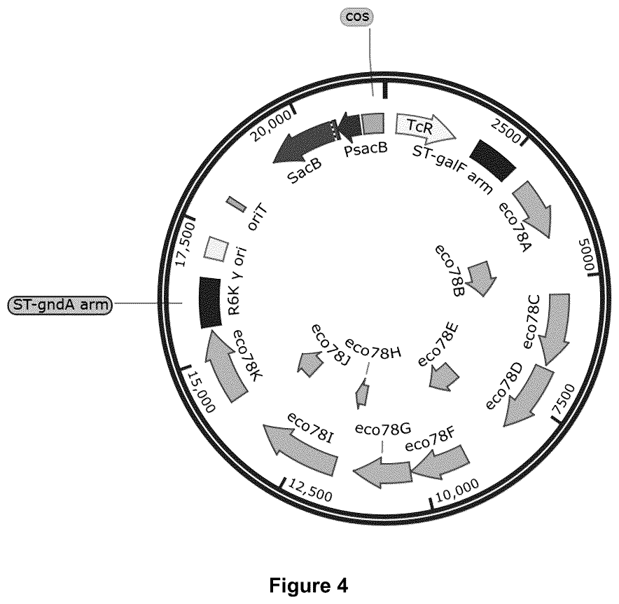

A deletion-insertion mutation Δ(rfbB-rfbP)::eco78ABCDEFGHIJK for the serotype conversion was introduced into the RASV strains by the gene replacement method [Kaniga et al, 1991] using a suicide vector pCHSUI-APEC-O78. Two DNA fragments, ST-galF arm (831 bp) and ST-gndA arm (931 bp) were amplified by PCR from the wild-type X³⁷⁶¹ using the primer set 3 (CHB250 and CHB560) and set 4 (CHB251 and CHB559), respectively. A 12,900 bp DNA fragment containing APEC serotype specific O78-antigen gene cluster (rfb_{O78}: eco78ABCDEFGHIJK) (FIG. 3) was amplified by PCR from the wild-type APEC strain X⁷¹²² using the primer set 5 (CHB561 and CHB562). These three DNA fragments, ST-galF (831 bp), APEC-O78 (12,900 bp), and ST-gndA (931 bp), were assembled with the Spel-digested pCHSUI-1 using GeneArt™ Seamless Cloning and Assembly Enzyme Mix (Thermo Fisher Scientific) to generate pCHSUI-APEC-O78 (FIG. 4).

To replace tra (traM-traX) genes on pAPEC-1 with asd gene, the three DNA fragments, Δtra_L-arm (811 bp), Δtra_R-arm (766 bp), and asd (1584 bp), were amplified by PCR using the primer set 6 (CHB541 and CHB542), set 7 (CHB543 and CHB544), and set 8 (CHB550 and CHB551), respectively. These three DNA fragments were assembled with the Spel-digested pCHSUI-2 using GeneArt™ Seamless Cloning and Assembly Enzyme Mix (Thermo Fisher Scientific) to generate pCHSUI-APEC-1-Δtra::asd.

To delete the antimicrobial resistance (AMR) genes on pAPEC-2, the two DNA fragments, ΔAMR_L-arm (830 bp) and ΔAMR_R-arm (811 bp) were amplified by PCR using the primer set 9 (CHB535 and CHB536), and set 10 (CHB537 and CHB538), respectively. These two DNA fragments were assembled with the Spel-digested pCHSUI-2 using GeneArt™ Seamless Cloning and Assembly Enzyme Mix (Thermo Fisher Scientific) to generate pCHSUI-APEC-2-ΔAMR.

A 1,052 bp DNA fragment was amplified by PCR from pAPEC-3 using the primer set 11 (CHB546 and CHB547) and assembled with the Spel-digested pCHSUI-1 using GeneArt™ Seamless Cloning and Assembly Enzyme Mix (Thermo Fisher Scientific) to generate pCHSUI-APEC-3.

### Chrome Azurol S (CAS) Assay

To assess the production of siderophores, the CAS agar medium was prepared as previously described [Schwyn and Neilands, 1987]. The ternary complex chrome azurol S/iron(III)/hexadecyltrimethylammonium bromide is a blue compound and serves as an indicator. When siderophore (a strong chelator) removes the iron from the dye, its color turns from blue to orange. The RASV strains were inoculated on the blue CAS agar medium and incubated at 37° C. for 24 h and then further incubated at room temperature for 3 more days. The orange halo formation by reaction with siderophores was recorded daily with visual inspection.

### Preparation of Antiserum to O78-Antigen

To raise chicken antibodies to APEC O78-antigen, ten 3-week-old SPF chickens were injected with an inactivated APEC strain X⁷¹²² with aluminum hydroxide as the adjuvant. Three weeks after the first injection, the birds were boosted. The serum was collected at 8 weeks and tested the ability to interact with O78-antigen using LPS immunoblot analysis.

### LPS O-Antigen Detection

Bacterial cells were grown overnight in SBM broth. The culture samples were adjusted to the same cell density (OD600=2.0) and 1 ml of each culture was pelleted by centrifugation. LPS samples were prepared as previously described [Hitchcock and Brown, 1983]. LPS samples were run on 12% SDS-PAGE gels, transferred to nitrocellulose membranes, and subjected to western blot analysis. Membranes were first incubated with a specific anti-O-antigen antibody followed by a secondary antibody (goat anti-rabbit IgG conjugated with alkaline phosphatase, Thermo Fisher Scientific). LPS bands were visualized using a chromogenic substrate, 1-Step NBT/BCIP (Thermo Fisher Scientific).

### Example 3: In Vitro Results and Discussion

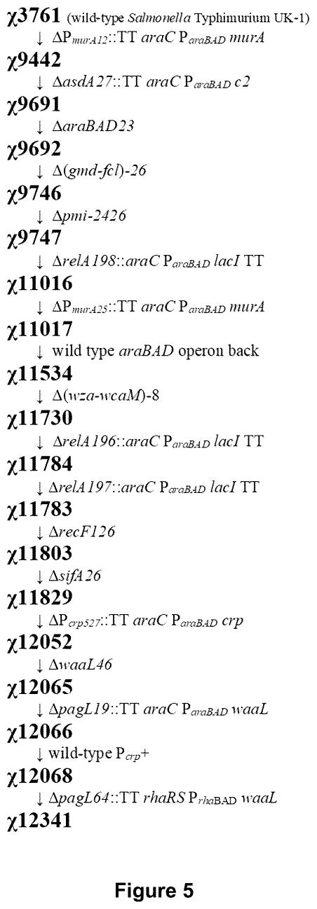

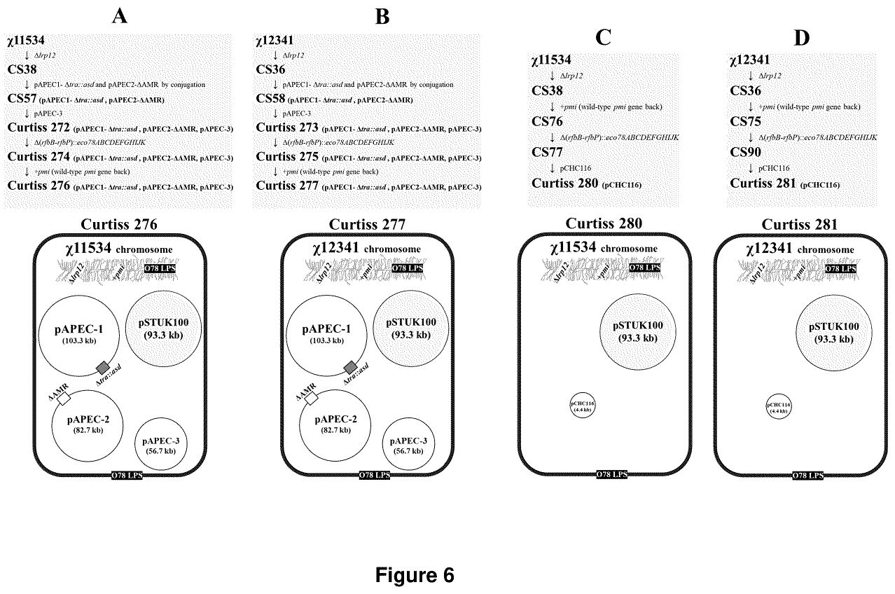

The APEC strain X⁷¹²² (O78:K80:H9) harbors three large virulence plasmids (FIG. 2), pAPEC-1 (103 kb), pAPEC-2 (83 kb), and pAPEC-3 (57 kb), and all three are known to have significant virulence attributes [Mellata et al, 2010]. Therefore, the virulence factors encoded by these large virulence plasmids would be potential target antigens to induce protective immunity against APEC. To develop RASV vaccines against APEC infections, all three APEC virulence plasmids are introduced into the RASV strains by conjugation (biparental mating) to display APEC antigens (FIG. 1). Two RASV backbone strains X¹¹⁵³⁴ and X¹²³⁴¹ were used as starting strains. These X¹²³⁴¹-lineage strains (FIG. 5) already have multiple mutations for the delayed-lysis system to ensure antigen presentation and biocontainment in vivo [Curtiss 3^{rd} and Wang, 2018]. The construction of the RASV-based APEC vaccines is summarized in FIG. 6. The virulence plasmids pAPEC-1 and pAPEC-2 are self-conjugative with genes responsible for the conjugation machinery on the plasmids. However, pAPEC-1 doesn't have a selection marker on the plasmid while the plasmid pAPEC-2 has the streptomycin-resistant (Sm^{R}) gene as a selection marker in the AMR area of the plasmid (FIGS. 1 and 2). For the conjugation selection of pAPEC-1, a suicide vector (pCHSUI2-Δtra::asd) with a chloramphenicol-resistant (Cm^{R}) marker gene was constructed and inserted into the pAPEC-1 plasmid before conjugation. This suicide vector pCHSUI2-Δtra::asd was also designed to introduce a deletion/insertion mutation Δ(traM-traX)::asd at the suicide vector removal step after conjugation. The resulting plasmid pAPEC1-Δtra::asd will be no longer conjugative and allow the Asd-RASV strains can grow without diaminopimelic acid (DAP). The virulence plasmid pAPEC-3 doesn't have genes responsible for the conjugation and selection marker on the plasmid, so a conjugative suicide vector pCHSUI-APEC-3 with the selection marker (Tc^{R}) gene was constructed and inserted into pAPEC-3 for the conjugal transfer and selection. All three virulence plasmids, pAPEC-1, pAPEC-2, and pAPEC-3 with the selection markers were transferred from the APEC strain X⁷¹²² to the RASV strains by conjugation. Then, the suicide vector part including the selection marker was removed from pAPEC-1 and pAPEC-3. The AMR genes on pAPEC-2 were deleted by the gene replacement method using a suicide vector pCHSUI-APEC-2-ΔAMR. The RASV-based APEC vaccines, Curtiss 272, Curtiss 273, Curtiss 274, Curtiss 275, Curtiss 276, and Curtiss 277, harboring all three APEC virulence plasmids have been verified by PCR and whole genome sequencing. This is a unique feature of the APEC vaccine formula which has never been done before.

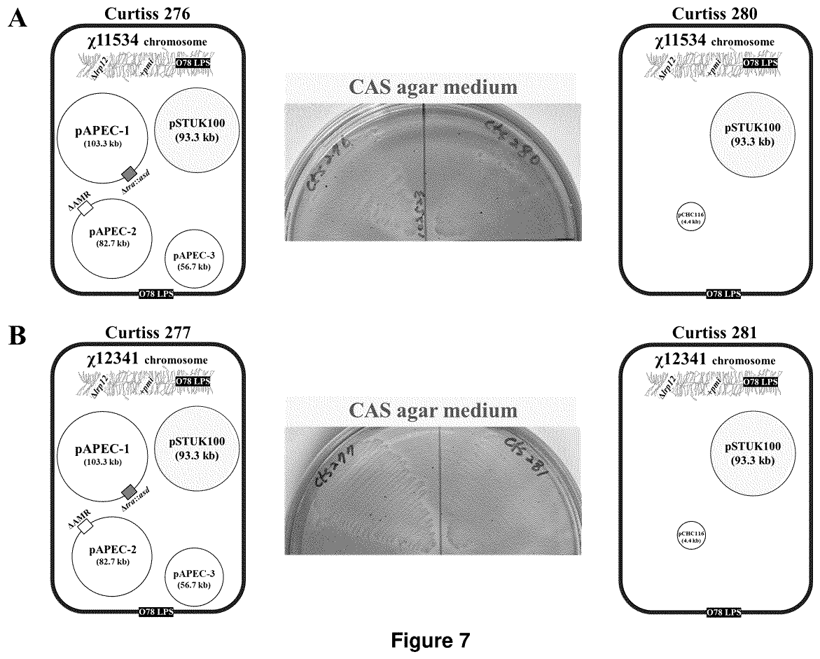

The bacterial iron uptake system is an important virulence factor for pathogenic bacteria [Braun, 2005; Fischbach et al, 2006; Griffiths et al, 1988]. The plasmid pAPEC-1 possesses four iron acquisition systems (iutA iucABCD, sitABCD, and iroBCDN) [Mellata et al, 2009; Mellata et al, 2010]. To test the expression of pAPEC-1 iron acquisition systems, siderophore production was assessed on the CAS assay agar medium [Schwyn and Neilands, 1987]. As shown in FIG. 7, the RASV strains Curtiss 276 and Curtiss 277 harboring pAPEC-1,2,3 showed a much bigger orange halo (representing the level of siderophore production) around the colony than Curtiss 280 and Curtiss 281 harboring the control empty plasmid pCHC116.

It has been reported that the 0-specific polysaccharide (O-antigen) biosynthesis (rfb) gene cluster is located between galF and gnd genes in E. coli [Amor and Whitfield, 1997; Bao et al, 2018]. Attempts have been made to construct RASV strains that will produce APEC 0-antigens (O1, O2, and O78) which are predominantly associated with APEC infection cases [Han et al, 2017; Han et al, 2018; Han et al, 2021; Roland et al, 1999; Roland, 1997]. Han et al (2021) introduced a plasmid harboring the APEC O78-antigen gene cluster (rfb_{O78}) into the RASV strain with a Δrfp mutation. However, the resulting RASV strains showed poor O78-antigen production. In a different approach, the cya gene in the RASV strain with a Δrfc mutation was replaced with the rfb_{O78} gene cluster from the APEC strain X⁷¹²² [Roland et al, 1999; Roland, 1997]. This resulting RASV strain also showed a weaker production of O78-antigen in comparison with APEC strain X⁷¹²².

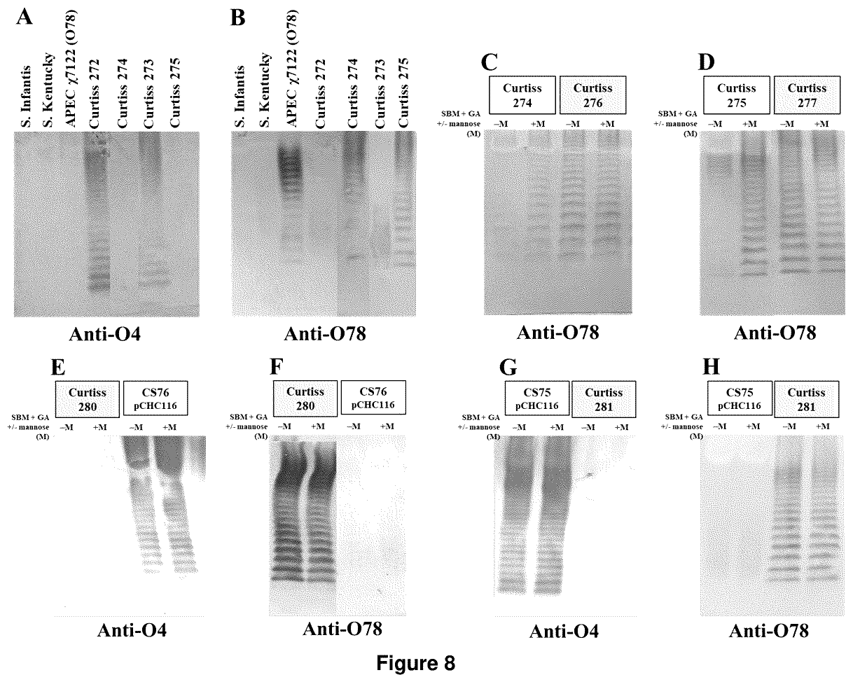

The present invention is directed to a method for the inter-genus serotype conversion by replacing the Salmonella O4-antigen gene cluster (rfb_{O4}) in RASV strains with the APEC O78-antigen gene cluster (rfb_{O78}). To design the conversion of the RASV serotype (O4) to the APEC serotype (O78), the nucleotide sequences of the genetic loci for the colonic acid and LPS synthesis in the S. typhimurium (UK-1) and APEC strain X⁷¹²² are compared (FIG. 3). The serotype-specific O-antigen gene cluster between galF and gnd genes does not share any significant sequence homology while the colonic acid coding area and part of the LPS coding area have significant sequence homologies at both nucleotide and amino acid sequence levels (>90%) (FIG. 3). With this notion, a positive-selection suicide vector pCHSUI-APEC-O78 was constructed (FIG. 4) to replace the entire serotype-specific gene cluster (18 kb DNA fragment between galF and gnd genes) in S. typhimurium with the entire serotype-specific gene cluster (13 kb DNA fragment between galF and gnd genes) in APEC strain X⁷¹²². The serotype converted RASV strains using this suicide vector are Curtiss 274, Curtiss 275, Curtiss 276, Curtiss 277, Curtiss 280, and Curtiss 281 (FIG. 6). All of these RASV-based APEC vaccines successfully produced APEC O78-antigen but no longer produced S. typhimurium O4-antigen (FIG. 8). This serotype conversion approach by replacing the entire Salmonella O4-antigen gene cluster (rfb_{O4}) with the APEC O78-antigen gene cluster (rfb_{O78}) has not been done before. Additionally, the phosphomannose isomerase (pmi) mutant will have a defect to produce LPS O-antigen in a mannose limitation environment like in vivo since the phosphomannose is a key building block for the LPS O-antigen production. The pmi deletion (Δpmi) mutation in Curtiss 274 and Curtiss 275 was replaced with the wild-type pmi (pmi+) gene using the suicide vector pCHSUI-pmi to generate Curtiss 276 and Curtiss 277, respectively. The pmi+ resulting strains Curtiss 276 and Curtiss 277 showed mannose-independent LPS O78-antigen production while the parental pmi mutants Curtiss 274 and Curtiss 275 showed a significant reduction of LPS 0-antigen production in the absence of mannose (FIGS. 8C and 8D). Similarly, the pmi+ strains Curtiss 280, and Curtiss 281 also showed mannose-independent LPS O78-antigen production (FIGS. 8F and 8H).

Leucine-responsive regulatory protein (Lrp) appears to be an antivirulence regulator in Salmonella since the deletion mutant (Δlrp) showed enhanced cell invasion, cytotoxicity, and hypervirulence in BALB/c mice [Baek et al, 2009]. To improve the invasion activity in vivo, the Δlrp mutation was introduced into the RASV strains by gene replacement method using the suicide vector pCHSUI-Δlrp (FIG. 6) so that the resulting RASV strains can exert enhanced immunogenicity.

### Example 4: In Vivo Efficacy of RASV-Based APEC Vaccines

To assess the efficacy of the RASV-based APEC vaccines in this study, SPF chickens will be spray vaccinated on Day 0 after hatching, boosted via drinking water on Day 14, and challenged with a virulent APEC isolate on Day 21. Then the clinical signs and survivability of birds will be monitored to evaluate the protective immunity of the vaccines in comparison with unvaccinated birds.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

Sequences

Nucleic acid sequence of rfb_{O78} gene cluster (eco78ABCDEFGHIJK) (SEQ ID NO: 4):
ATTTtgaaagcttgatcggatatgacggctgcgaaaaattggaataattgcaggttattttgaatatgggggatttctaaaacaccccattaaca
atgtgttgacaattgattttgaccaagttgttaatgtttttcttgcgttttcaattggttgattagataatattatatagatctataatctattc
tagttgggttgggttgggttgggttgggttgggttgggttggggaaattgttcgatcggaatatatagtttctggtgttctgatagccataagtc
aggggcggtagcgtggatttttattgcgtaaaaacgtcagtaaacatagaggtttaatgaaaaaaatactattaatatccatgagtttgggaaag
ggcgattatggtggtggtatagtatcaaatacaaacttttttgctttaaaggaattagagcattatgagttattttcagtaggtattgtaaaaaa
caccaacgatgcaccaaactttattaacatggttttgcctggcaacgcgagtaaattctcaactgcaattaataatatattagggtttgccgggc
agttaaataataaaactacgaaaaacataaaatacataattgacgagtttgagcctgatattgtataccttgattcaagccttctggggtgtata
gcttcttattgcaagaaaaaacataaaactattcaaataataacgttctttcataatattgagtttgattttgaaattgcacgtattatgtcagg
gttattgcatttttttccctcgttaatatccacaactctcgcagaatatgcggctgtaaggtatagtgacaagattatcgcattacataaaaaag
attcttttagactagaggaaaaatatggaaggaaggctgattatatcgtacccgtttgcattaaagatacacaaagagaaaaaagttttaaactc
gtcaacgaaaagaaaaaagaaggcaaaaagttaaaagttggctttataggtacagctttttttgcaaatgtagagtctgctaagattatatctca
atacattgcgccaaaggtggaggggattgctaatttttatatatgtggcaatggatttgaaaaatataaagcattgaattcaactaatgtaaatg
tttcaggctatattgattctttggatgatttctataatgaaatggatgtgatgatatttcctatttttagcggcgcaggaatgaaggtgaaaata
gcagagtcattaatgtataataaaccaattctagcttctgcatttgcattagttggttacgaaaaaataatagatggaactaatgtgattagctg
tgagtctcacgaatcttttgtttatcatataaaacaatttagaagggataataacacattatataataggaagtcttattataaatatttctctg
ataaagcatgtctgcattattttagaaatatattacgagaaattgttaataacgaatagatagatggactaataacgtcatgaatataatacaat
tatcaaaattctaccctccgatatttggtggaatagagcaagttgcacaggacataaccgaagggatgcaaaatttgtgcaatgttgatgtttta
agcgtaaataacagttctaaaacaatatattgtaagaatataattcgagcttcattactatttacactattatctaccccagtatcgattagcta
tatactaatctggagtagaatcagaaataattatgatataattcatgttcatttgccaaatcctcttgctattattgcactgttgctgttcccgc
ccaaagctcccgtcgtagtgcattggcatagcgatatagtaaaacagaaaatagcgttaaagttttttcggcctttacagaatttatttttgaat
aaaagtagaaaaaattattgtgacatctgaaatttatgggagttcttctcctcaattcaaagatttcaagataaaataatttgtataccgatcgg
tataaaatcagagcgattacctaagaatgaaacgctattaaagcatctaaaagaaaaatataaaaataaaaagatagttttctctcttgggaggc
tggtttattacaaagggtttgaaaatctagttaatgcagctaactttcttcctgaagatacaataatccttattggtgggtgcggtgagttatat
gacgagttggctgatagcattttatccaataaactagaagggaaagtggttttgctgggggagataaaatatgaacagctttctgcttattatca
ggtttgtgacgtattttgtcttccttccattcatgaatcagaagcttttggcgtggttcaaattgaggcaatgagctatggaaaacctgttgtat
ccacaaatattaaaaatagtggggtcccatgggtaaatgaaaatggtatttcaggtgttgtagttgaacctaatgagcctcatgaactagccaaa
gctatcttgacaattttaaataatccagctggtttttccttaggggcattagagcgatatcgcaaattattcactagagataaaatgatctcaag
tttgattggactttatcagaatattaaaataggaaatgaaaaagaatgatcttacctgttattctggccggtggtgcaggtagtcgcctttggcc
actttcccgcttactttaccccaaacagtttttatgtctgaaaggcgagctttctatgctgcaaacaactatctgccgcctgaatggtttggggt
gcgaaaatccggtggtgatttgtaatgagcagcaccgctttattgttgcggaacagttgtatcaactgaacaaactcaccgagaacattattctt
gaaccggcaggacgtaatactgcacctgctattgcgctggcggcgctagcaacacaatggcataaaccagatagtgatccgttactgttggtgct
ggcagcggatcatgtgattgctgatgaagacgcattccgcgccgctgtgagtaatgctatgccgtatgccgaagcgggcaagttggtgacttttg
gcattgtgccaaggcatccagaaacaggttatgggtatattcgtcgtggtgcagtgtctgcgggcgagcaggagggagtagcctttgaagtggca
cagtttgtcgaaaaaccgaatctggaaaccgcgcaggcctatgtggcaagcggcgaatactactggaacagcggtatgttcctgttccgcgccgg
acgctatctcgaagaactgaaaaagtatcgtccggatattctcgacgcctgtaaaaaagcgatgagcgccgttgagccagatcttaattttattc
gtgtagatgaagaagcgtttctcgcctgtccggaagagtcggttgattacgcggtaatggaacgaacagcggatgctgttgtagtaccgatggat
gcgggctggagtgatgtgggctcttgggcttcgttatgggagagctgcactcacacagctgaaggcaacgtttgccgcggtgacgttattaatta
taaatccgaaaacagttatgtgtatgccgaatcgggcctggtcaccaccgtcggggtgaaagatttggtggtagtgcagaccaaagatgcggtgt
taatagccgatcgcagctcggttcaagatgtgaaacatgtggttgagcagctcaaagccgacggtcgacacgagtatcggatgcatcgcgaagta
taccgtccgtgggggcaatatgactctatcgatgcaggcgctcgctaccaggtgaaacgcataactgtgaaaccgggcgagggcttgtcggtaca
gatgcatcatcaccgcgcagaacactgggtggtggttgcgggaacggcaaaagtcactattaatggtgacatcaaactgcttggtgaaaatgaat
ccatctatattccactgggggcgacacattgtttggaaaatccaggaaaaatacctttagaaataattgaggttcgctcaggttcttatcttgaa
gaagatgatatagttaagttttatgatcgttatgggcgagattaatttcattactgaagttgctaattgtttaacaaaatacttctgtgtatttt
ttcatataagtttgtaaatatgactagattaagctgctttaaagcctatgatattcgcgggaaattaggcgaagaactgaatgaagatatcgcct
ggcgcattggtcgcgcctatggcgaatttcttaaaccgaaaaccattgttttaggcggtgatgtccgcctcaccagcgaaaccttaaaacgggcg
ctggcgaaaggtttacaggatgcgggcgtcgacgtgctggatatcggtatgtccggcacagaagagatctatttcgccacgttccatctcggcgt
ggatggcggcattgaagttaccgccagccataacccgatggattataacggcatgaagctggtgcgtgagggggcccgcccgatcagcggcgata
ccggactgcgcgatgtccagcgtctggcagaagccaacgactttcctcccgtcgatgaaaccaaacgcggtcgctatcagcaaatcaacctgcgt
gatgcatacgttgatcacctgttcggttatatcaatgtcaaaaaccttactccgctgaaacttgtgatcaactccggaaacggcgcagcaggtcc
ggtggtggacgccatcgaagcccgctttaaagccctcggcgcaccggtggaattaatcaaagtgcacaacacgccggacggcaatttccccaacg
gtattcctaacccgctgctgccggaatgccgcgacgacacccgcaatgcggtcatcaaacacggcgcggatatgggcattgcctttgatggcgat
tttgaccgctgttttctgtttgacgaaaaagggcagtttatcgagggctactacattgtcggcctgctggcagaagcgttcctcgaaaaaaatcc
cggcgcgaagatcatccacgatccacgtctctcctggaacaccgttgatgtggtgactgccgcaggcggcaccccggtaatgtcgaaaaccggac
acgcctttattaaagaacgtatgcgcaaggaagacgccatctacggtggcgaaatgagcgcccaccattacttccgtgatttcgcttactgcgac
agcggcatgatcccgtggctgctggtcgccgaactggtgtgcctgaaaggaaaaacgctgggcgaactggtgcgcgaccggatggcggtgttccc
ggcaagcggtgagatcaacagcaaactggcgcaacccgttgaggcgattaaccgtgtggaacagcattttagccgtgaggcgctggcggtggatc
gcaccgatggcatcagcatgacctttgccaactggcgctttaacctgcgctcctccaacaccgaaccggtggtgcggttgaatgtagaatctcgt
ggtgatttaggacttgttgaaaagcagacaacatatatcattaacttattaacaaccccccccaatgatgaaaagtgaaaatattacattcgtag
tgcaaggtccagtcagaaatgaaacaaaagatactttagaaagtattaggctaaattttaaagaagctaaaataattttgtccacatgggagggg
agtaacgttaaaggtctttcatttgacgatattgtttttagtaacgatcctggccctttaaatataaaaagaggtactaaaatagttgctcagga
aaatacaaatcgtcaaataatttcaacataccaaggtttaattaaggttcgaacaccatatgcggttaagttgagaacagatacgccattgcgaa
atgataatattataaaaaaatatataaaagctcaagcatatgggagagattataatttttcatacttagaggaaaggattcttgtatcatctata
aatactatagatccaaaaagctatattcaattcccataccatatttctgattggatctactttggtaaaactaacgatttattaaaaatttggga
tggggaattaatagatgataatgattattttactgagcaggatataaaagaagagattagttgcaaaagaggatttgagtttggaagatacacag
ctgaacagcttgttctatactcatttttaaaaaaaaataaaattagcgaatataaacattattgtgatacaaataatgacagggttgaaaaggta
ttaaaagtaatcctttctaatttctattgcgtaagtcctaaacaaatgggactctgttttgataaatactcagacttaataacccctagaatgaa
ttacaggagtctccgtagtttcttagggttttattttgttacaattaatgagagagtttggcgaaattattacagccaattctataaacttgaca
ctacaatatcagaaaaaatagaaatatgtgcacgcaggatatttataaagatcatgaagaagaggaaaaaatgagtaaaagcatgttatatatct
acacacataatggttacaatgatgcatttttaaaaaaagatgtagggatggtaccatctgttatgttaaatggtgggaaagttgattcgctaatt
tattgtaaagaaaagaaaggaaaggctgtttttaataatttagaaatatattttactggatgtggatggtttattagtaacttcaaggcgatact
gtttctttgtcgaaatgcaatatttaatagacatttagatttattgatatttcatctcagccttagaaatatcgtgcctgtgttaacatataaac
ttttatttaaaggggaattaatatgtaagatggatttaaatactgaaagtgccatcgaatattctgataaaagccataaaataaatgggatcaaa
aaaaaactaataagaatgattatttcccgcacagatcaattttatgttgagactataagaaattataacataataaataaaggcatttttgatgt
gccgatttcaggaaagttaaaattaatgcccaacggagttgatgctaaattagttagggaggttattaaaactgaaggtcctgatgtatttaaag
aaaatacaataacaataatctctcgccatgcttgtgaagcaaaggctccaaacagaatattcgatgtaattgacgtaatgtctgaattgaaactt
aataactggacattgcaaatattgggctatttcccaacaatatttacggatgctctaagaataagagctcttgaatgtggagtgaatatcatatg
tgacggttataatttagagctctatgatgtgtataaaagattatttaaaagtaagatgtttatatgtctttcgctgcaagagtcatattgtatat
cactagttgaagctgctgtatttaataatcatataatatctacaaatgttggagtagcagaagatctttcattaaaatacggtaatattaaaatt
atcgaaaattattcgaaaaatttactatccaaaactattgaaaagacaattaaaaatgatattaaaataaaaaaagcaagcatttctgatagaat
gttagactcatattcttgggataatatcataacaggggtcatgagcgaataatatgggagtatatttcattttaatgacagtggcactattgatc
tctattatcgtattattccacaatgatgttaaatttacgaatgtatatatatatacttatattttactgctctcattacttgctggtttaagatt
tcataccggctgggattgggaagcttatgactattttttttatgagctgacaagttctaatttatttgaaataagcaaagcaaatgtttttaatt
atgaaccagggttcgttttactatcttatatatctgttttattgcacataccaccatttctctttttctctttaataaccgttacattaattata
aagtcggcttataagtatttagggaactttgtttacatttttttattaatatatttatattacggttattttcataatttttctattgtacgaca
aggtgttgctgctgcgttattcgcttattccattcgttatttgatagcgaaaagctataaatattatatattaatatttgtcgctgcattattcc
atatgtctgctattatgttattaattgtcccatttctttataaaatcgctaatcgtataccattttccgccctattgctattttcattgttaatg
gtttatacctctgtaagtgagctcataggtatgaaatcattattgtcaggaataaatgcactaaatatatatatcaataatagttctttaagcta
taaagttggtttttcgtttaaatatttggagttattaatcattctagttctattttataataagagattttcagaactgattgctagcaaattta
gtaaaaagcaatattatgtatttagatgtttgattgtaattgaagtgatgatatattcgcttttcaatgattttagcattatttatgaaaggcta
actgtttacttcgaattttctcatgctatatgtatagcaatgattatatcagcctttaagtataaaagagttcagttgtttttactgttaatttt
attgtctcttatttttgtaagatattatcaattattcaattctcctgttcgtgtcgatggagaaatgacacattacgaacgatttgaaaactatt
gttctgtttttaacacaaaagattgtcaaaggtaacaatgaagcgaattattatagacttggataatactatcacaatccataacgatgaaccat
acggtaaaaaaccagtaaatcaagatgttgtaaataaaatgaagatgtataaagagctgggttatacaatcacaatatttaccgcccgtaatatg
aatactttttcaggtaacattgggaagataaatgttcatacaatgcctgttataattgactggttaaatcgtaataatattccatatgatgaaat
tattgtgggtaaaccatggtgtgggacaaatggattttatgttgatgataaggcgattaggccttcagagtttataagcttgtcagaaacggaga
ttgctgaactactggataaggaggtaaaaaaatgattctaataaactctgctgcttacgttaatgacgacctaagagctgaattcggtttgctac
caccgtgctttttaccccttggtaatagaagactatatcatcatcagattgagtcgttaaggaaagcttttcctgaagaaaaaatatatttatct
ctgccggaagagtatcacattggtaggtatgatcaacaatactttaatgaaaatgatattacaatactaaaaacttcttatactttatcacaagg
acatagtctgtatcggttgttaaatgattttaacttaatcgaatgtgataaactaattattaattatggtgatactttttatcatacctttgaag
ctacgagtgatgattatttttatgtatctaataacatcggctactataaccgagcatctgttgtaattaatggtgataagcttattataaataaa
gataatccagctgttgataatggtgagttggttatttctggtttcttttgtttcagtaatattgggaggttatgcgaatgcttgaaaaagatgaa
ctatgattttctgtcgtcttctgaattgtattactcgtcggttgcaacaaaagttttaaaaaatgatcattggtatgattttggtcatttaaaca
gcttttttacatcaagaacgagtatcactactgagcgtgagtttaatagtcttcaaataaataatttttgtgtgataaaaaaaagtaagaagaaa
gcaaaaatgctaggcgaagcgaattggtttttgagtttgccgcctgcattgagtatcttcactccgagggtttttgatgttagagaagataatga
ttatgcagagtatacaatagaatatctttataatcttcctttgtctgatatggcagtgttttgtgagttacctaagtcatgttgggtgaatgtgt
ttaactcgtgtcgagatttcttaaatagatcaaaagaatactctaatagtaaaaaaacgctagatatgtctgaactggcatcatatgataaattg
tactatgataagaccctaaaaaggttacatgattttgaattgacaacaaacatacaattgactaaagatgttatatttaatggtaaaagatttcc
atcgataaaaaaatagcagaaatatcatcacgctttattaacccaactaagagtgatgatgttataattacacatggcgatttttgctttagtaa
tatattatacgattttagaagtcagagaattaaattaatcgatcccagaggtattaactataacaatgagttaagcatatggggagatataaggt
atgacttggctaagttatgtcactcggtaattggatgttatgatcttattatcgctcagaaatttttgctggaatacgattatgtagataacaga
attgaatttgaactatatgatgataactacagcgacatagtgaattcatttttaggcgtgtttaattcttttgaagaatttgggtatactattga
agagatacaatgtataaccatacatttatttttatcaatgttgccgcttcattctgattctaaacttagacaagaagcatttatagcaaatgctt
atagactgttcagtttgttaaatttggagtatttatgacaacgattgtaattcctatggcaggacaatcatcacgcttttataaagcaggttatg
atgttcctaagtataagttaaaaattaataacaaaagcgtattttttcattcactacgaagttttctaaaatttaaggaccatttttttctaatt
ataggtattaaaggcatactagatgaaaagtttgttcaatatgaaatggccgaattaggtataaatgattttgaaattgttttgctcgaacagca
aacaaatggtcaagcagagacggttgtgaaaggagtaaaaaattgcaagagagatattagtgacattttaatttttaatatagatacttttcgca
gagagattaatttgcctgataattttagtttgaaagattgctctgggtatttggaaacatttttagggtcaggtgagaattggagtaatattatg
cctgaatcagatgtccctaatcaagtaaaaatgactgcggaaaaacaaaatatatctgaatattgttgcactggtttatattattttggtgatta
taagttgttgaatatagcttataataactggctagaaaatgaagatactactaaagaggtgtatatcgctcctctatataatcatttgattacat
gtgggcacaaaattttttataccgttataaacagatgtgatgtggtgttctgtggtgtgcctaatgaatatgaaaaaataaagaatgggtttgac
tggaaatgagacagcacagaaaattagtcagttatgctgtgattattacttttttgtttatacctatacaagttattctgtcgttaatttttaac
tattatttagctaaaaacgttgctctggatgttttaaatgcttggtatatgttttttgcactcatttcccttttttctttgttggaattttccta
tcccatattagcaattaatttttttaacagctatcagggaaattgtaaaggagctctgtctttcttcataaagaaatcattaggggtagtaatac
cccttcagatcatatttgttgtaatttatagtttatttgttaacccgctttttatttattttggtatgggtttggtggtacgtagtatatcgaat
attattaatgcatgtgtatatgcaaaacaaaatattattatcgataaagtttatcgatttctgtacgccataattatgccaactgctttcctttt
cttgttttatcattctgataaaaacattgatttgtatgatttaactcttgtatggtttttgagctcactattctgcttaatatattctttgtcat
ttttactccttaagcataagaagtctgtcaacaggagcgaaaaatcattccatctaccattcaagtataaccttaagcttttttttactgttttt
cctgcaattttcatctatacattgtctatatattatttgaaaatatttggttcagaaagcagttctagttctactattatatatggtttcttttt
gcaaatatttaatgtttataatttgataattattttggttgcaagttatttgatgcctacgctctcaaagcgtttccatgatggtgttgatgtta
tccctctcacacttaaactattagatatcagtgtacttatatctgttatttctacttcttttgtggcattgttaggtattcctgttgcggagtat
attcttaaggggaaaatagatgtaatatcctactattatatagtagcaataattattatattctatattgaaacatgtcaggttattctaacctc
aattggcactgccattggtatttacgattttcataggcaaagtattatatcagcagttatggttttaattctgtcatgttttttaatacctcatt
acaatgctgagggtttgatgtatagtatcattatatctcaattctttacatgctttatttataaccctaaacgtgttataaataaaattggatta
gataaagttaattatattaaaagattaatggtgcactttttgtttatgttgactcttatttttatggggttatattttcaaaatgatgatatatt
gccgagagttgtgattctttcttttatcacattgattggtgtttgttttctctacccaaatgtgaaaaaaataattgttgatttcttgtagtttt
ttaattttatttgcattttaaatggaaaatcttgttctatcttagaaaggagttaacaatgctgaagcaacagatcggcAAAT
Nucleic acid sequence of pAPEC1-Δtra::asd plasmid (SEQ ID NO: 1):
ttggctatcctaagcagccgattatgcgttgaggctttcagtgatggctgatatagcaacaatgagaatgaaggctctgcacttctgggataaacacggtatt
tctgcagcttctgaagcctttggcgtatcctgccgcacgctttactggtggcgtcagttactgaacaagggaggacctgaggggctaatcccgcacagtaagg
cacctctggtgcgacgaaaaaagcactggcatcccgatgtgctgaaagagattcgacgcctcaggacagagctgccgaacctcggtaaagagcagatttttgt
tcgcctgaagccctggtgcgcataacgccatcagacctgcccgagtgtttccaccatcggcagaatgatcgccaccgcacacgataaaatgcggatgatacca
gtacgtctgagctcgcggggtaaggctctgcttgtcaaaaagcgaaccaccaaacccccgcaggccaaagcactaccgtccggtaaagacaggagagctcatt
gggatggacgccattgagctcagaatgggtgaaatgcgtcgctatatcattaccatgatcgacgaacacagcgattacgtactggcgctggctgtgccgtcgc
tcaacagtgatatcgtcaatcactttttcagccgtgcagcccggctgttccctgtcggtatcagccagatcatcacagataacgggaaagagttcctgggaag
ctttgacaaaacgctgcaggaagccgccatcaaacacctctggacctatccctacacgccaaaaatgaacgctatctgtgaacgttttaaccgggcattaaga
gagcaattcattgaatttaatgaaattttactctttgaggatctggcgttgtttaacctgaaactggcggaatatctggcgctgtataacagcaaaagactcc
acaaggcgctcgcactaacgacgcccgtggaatatattttaaaagagaacaagaattgcaatatgtggtggacccatacacagcaaacgcacaggccgccccg
gttgggtagatatgtgggatggctggttcagacttcaggatatggttgaggctggtgatgaagtctctttatcaggagatctgatcaagagacagccgcgcag
ggggattcactatgctggattcttttaacaaattctccgctaaccaaaaaatagactttttgcattcgtaaatgcgttaagagataatttttaaagttccatc
aatctcccggccagttcgtctcgcggcattactcactatccgcacaacttcagtaagatcatgactttttcaatcgttatctgttgaccaagctttgaaacca
ctagcgtcccgtcaacctgacctggaatcgtcgagaaccgcccatagttgtgaattggctagagtgtgatggaaagcaacgctaaacgacctagtcacacatt
ctcttttccctaacaatgaccatttagtgaaataacgtatccaaaatgatgacgtgaaagcctgacactggcagcgaaaagccgaaaaaactcacctggtcag
gagcaaaaatcggattattcgccaacagcgtaatatcagcagtagttctcgacacgcttgcgctatagacagaggatgaaacgctgctggtcggagttctcta
tacatttttggcgacggcagaaccgctcaccaatgccttaacgtctatcagccttccggtgagttggaacagcatgctcagcgtttgcaagatattctccacg
gtctgtagcagcgtaggtttcaatttaaatatccccagatagccctctgggagcttccctttttattaagttttaattaccgttgcaatagttaaatgatatt
ggtaattattatcattagcatttgtgtctgtgttgtttctggtaaggctgaaagggaacaaacatcgcctgtacaacggcgtttttacttacagaccagcgaa
ggcgagactatcaggatgatgcatcgtgtgtctaagaaatcacggttctttccagattcgttgttcggtctaacactgcacaaatgcgtcagaacatcccggc
gtagctgtccgacgccatttgggctgttcaacaggacaatggcgcgtaagcgttaatggaagtggggatacggagttccctgttcacctgtgtatgtcacgga
aaagtctctgactgagctgtccataacgctggtgaagcacaaatggcgcagcatctggtaaagcaggtttaacgaaaagtcgtgtcctgctgctggcatatga
gtgcgtcgactgcctgatttagatcgtcaagcggagagggattttctcatgcgtattttgtttgtaggcccgcctctctatgggctgttgtatcctgtgctgt
ctctggcgcaggcatttcgcgttaacggtcatgaagtgctgatcgccagtggaggacaattcgcacaaaaagcggcagaggccgggttggtggtgtttgacgc
cgcacccggtctggattccgaagctggttaccgccaccatgaggcacagcggaaaaaaagtaatatcggaacccaaatgggcaacttctcattcttcagcgaa
gaaatggccgaccatctggtggagtttgccggacactggcggcctgacctcatcatctaccctccgctcggagtcatcgggccgctgattgccgccaaatatg
atataccggtcgtgatgcaaaccgtaggcttcggccataccccctggcatatcaagggtgtaacgcgttcgctcacggacgcctatcgtcgccacaatgtggg
ggccacaccacgtgatatggcctggatcgacgtaacaccgccgagtatgagcattctggaaaacgacggtgaacccattattccgatgcagtagatctaatcg
cgtcccgtataacggtggcgcggtgtgggaaccatggtgggagagaaggcccgagcgtaaacgtctgctggtgagtctcggcactgtcaaaccgatggttgac
ggccttcctgggttatggattctgccagtgaggtcgatgccgagatcatcctgcatatttcagccaatgcgcggtccgatttgcgctcgctgccgtcaaatgt
tcgcctggttgactggatacccatgggtgtcttcctcaacggcgcagatggtttcattcatcatggcggagcaggcaataccctgacagcgctacacgctggt
attccacaaatcgtcttcggccagggagccgatcgcccggtaaatgcccgcgttgttgccgaacgcggctgcgggattatccctggcgacgtcggcctgtcga
gtaacatgatcaatgctttcctcaacaatcgctcacttcgcaaagcctctgaagaggtagcagcggaaatggcggcgcaaccctgcccaggtgaagtggcgaa
aagcctgatcacaatggtacagaaagggtaaccactacaccttcatgataatcatatgccagcacccatccaggagtcatgcatccataacaatgcgggaccg
gagcgtgctggcttttttgcggaacgatccggccagtggatcgtattttacagacggaggaggcttaatgcctgcgaatcacactcccacaccggctcagtca
tggatagttcgcctagcgcgcgtgtgctgggaacgtaagaaacttagtgtcatcgtggtggtagcgtcagtatcgactattttgctggctgcgctgacgccac
tgctgacaagacaggctgtgaatgacgcactggcgggcaatccggcccgcctgccgtggctggcctgcgggttactgttgatcgctttttttgatttcatcgg
taactatgtgcgccgtggttatgccgggatgctctcactctgggtgcagcataccctcagaggacgggtattcgacagtattcagaaacttgacggcgcaggc
caggatgcgctgcgcaccgggcaggtgatttcacggaccaacagcgatctgcagcaggtgcataccctgctgcagatgtgcccggtgccgctggcagtgttca
cttattacattgccggcattgccgtgatgctgtggatgtcccctgccatgacgcttatcgtcgtgtgcgtactggtatgcctggcgatcaccgcgcttcgtgc
gcgtcgtagggtcttcgcgcaaaccgggctggcctcggaccaactggcgaatctcaccgaacatatacgcgaggtgctggcacagatctcagtggtaaaatcc
tgtgtggcagagatgcgtgaaacgcactggctcgataggcagtcgcggcagattgtgcgtgtacgcatcggtgcggttatctcgcaggcgatgcctggggcca
ccatgctggcgctaccggtgctcgggcaaatcgtcctgctgtgctacggcgggtggtcggtcatgcacgggcggatcgatctcggtaccttcgttgcattcgc
cagcttcctcgcgatgctgaccgggccaacccgcgtactggcatcgtttctggttatcgcacagcgcactcaggcgtccgtggagcgggtgtttgcactgatc
gacacccgttcacagatggaggacgggacggagtcgattaacagtcaggttgtcggactggaactggagaatatgagctttgactaccaccatggcgacagac
atatcctcagcgatatctccttttccctgcgcgccggtgaaaccgtggcggtggtgggcgcatcgggttcaggaaaatcgaccctgttgatgctgctggcgcg
tttttatgatccctgctccggaaagatatggctcaacaccagcgaaggccgacaaaatcttcgcgatatcagactggaggcgcttcgtcgccgggtaggcatc
gtatttgaagatgcttttctgtttgccggtacggtggcggaaaatatcgcctatggccaccctcaggcaacggcggacgacattcgccgtgcggcagctgctg
caggagccagcgattttatcaacgccctgccgaaaggcttcgatagcctgttaaccgaacggggtacgaatctttccggcgggcagaggcagcgaatagcgct
ggcgcgggcgctcattactgcaccggacgtgttaatcctggatgataccacctcagcggttgatgctgttacggaagcggagattaataccgcgctgggtcgc
tatgctgacgaagggcatatgctgctggtgattgcccgacggcgttcaacacttcagctagccagccgggttgtggtgctggataagggccgtatggtggata
ccggaaccccggcagaacttgaagcgcgctgtccggcgttccgcgcactgatgaccggcgacagcgattttctggccacgtcccacaatagccacaacgaatt
gtggccggctgaaccagcgacacaagacgatgtaacggatacgggggataaaggttttgtcgcccgtatgacccgcgtaccggaaaatgcagtacagcaggcg
ctggccggtaaaggccgcaaagtcacgtcactactgaagcctgtggcgtggatgttcgtcatcgccgctctgctgatcgcactcgattctgcggcaggcgtag
gggtgctgatactgttgcagcacggcattgactccggtgtcgccgcaggcgatatgtcgaccatcggcctctgtgccctgctcgccctgagcctggtcattgt
gggctggtgcagttattctctgcagacggtcttcgccgccagagcggcggaatcagttcagcattcggtgcgcttgcgcagcttcggccatatgctgcgtctt
ggactcccctggcatgaaaagcatgccgattcgcgtcttacccgcatgaccgttgatgtggactctctcgcccgctttctgcaaaacggccttgccggtgcgg
ccaccagtctggtgacgatgttcgcaatcgccgccaccatgttctggctcgacccgttcctggcgctgacggcattaagcgcagtgccagtggccgcactggc
aaccatgatttatcgccgcctcagtacccctgcttatgcacaggcacggctggaaataggcaaagtcaacagcaccctgcaggaaaaagtctctggcatgcgt
gtcgtgcaatcgcatggtcagcaggaactggagggcgcccggctgcgcgcgttatcggagcgtttccgcgcaacccgtgtgcgagcacaaaaataccttgcag
tctattttccgttcctgacattctgcaccgaggcctcctatgccgctgttctgttagtgggagcttcgcaggtcgccgctggagaaatgactgccggggtact
ggcggctttcttcctgttgctggggcaattctatgggccagtgcagcagttatcagggattgtcgacgcctggcagcaggcgacagccagcggcaaacatatt
gatgaactactggcgacagaaggcactgagaacctcggctcctcttcggtcctccctgtcaccggtgcactgcatcttgatgaggtcacgttcagttatcccg
acagtcacgagccagctctgaacaaacttaccctgacgatcccagagggaatggttgttgcggtcgtcggtcgcagcggtgcgggtaagtcgacgctgattaa
gctgattgccgggttgtatttccccacgcacggcaacatcagaatcggtgtgcaaatgctcgatgatgcctcgctcgctgagtatcgtcgccagattgggctt
gtcgatcaggatgtagcactgtttagtagtgatattgcagaaaacattcgttattcacggccatccgccaccaatgaagacgttgaaattgcctcacagcggg
cagggctgtatgagatggtgtgcaatctgccgcagggattccggacaccagtgaataacggcggagccgatctgtccgcaggtcagcgccagttgattgcgct
gtcccgcgcgcaactggctaatgctcacatcctgctgctcgacgaagccacgtcatgtctggatcgcacatccgaagaacgactgatgtcatcgttaacagat
gtcgtgcatgccgggaagcactcggcgctgattgttgcacatcgtctgaccaccgcgcaacgctgcgatctgattgccgttattgataaggggttacttgcgg
aatacggaacccacgaacagctgttatctgcgggcggcctctatacccgcttatggcatgacagcgtcagcagtactgcgctccatcgccagcacaacatgaa
ggaggaaaccccgggatagttactggacacgtaatgtattaaaaacacagtcagaagcggcggtaccgtgaatagccgctttaattatttatactgacatcct
taatttttaaagagtatgaatgctgaacatgcaacaacatccctctgctatcgccagcctgcgcaaccaactggcagcgggccacattgctaaccttactgac
ttctggcgcgaagctgagtcgctgaatgttcctcttgtgacgccagtcgaaggagcggaagatgagcgagaagttacctttctgtggcgcgcccgacatcctc
tgcagggcgtttatctgcgtctgaaccgggtgacggataaagagcacgtagaaaaaggaatgatgagcgcccttcccgaaacggatatctggacactgacact
gcgtttacccgcaagttactgcggctcctattcgctgctggaaatcccccccggcactacggctgagacgattgcactgtccggaggccgtttttccaccctc
gccggaaaagccgatccgctaaacaaaatgccggagatcaacgttcgggggaacgcaaaggaatcagtgctgacacttgataaagctcccgccctgtcggaat
ggaacggcggcttccacaccggacaactgcttacctccatgcgcattatcgccgggaaatctcgccagattcggctctatattccggatgttgatatttctca
gcccctcgggctggtcgtgctgcccgatggtgaaacctggtttgatcaccttggcgtatgcgcggcaattgacgccgccataaataacgggcgcatcgtgccc
gtggctgtactgggcattgacaacattaatgaacatgaacgcactgagatactcggcgggcgcagcaaactgataaaggatatcgccggacatctgctgccga
tgattcgcgctgaacaaccgcagcgtcagtgggcagaccgttcgcgcacagtgctggccgggcagagcctcggcgggatcagtgcgctaatgggggctcgtta
cgcaccggaaacgttcggtctggtgctcagccactctccttcaatgtggtggacgccagaaagaaccagtcgaccaggcttgttcagcgaaaccgatacctca
tgggtgagtgagcatctgctttctgccccaccgcagggcgtacgtatcagcctgtgcgtgggatcgctggaaggttcgacagtgcctcacgttcagcagcttc
accagcggctgattaccgctggcgtcgaaagccattgcgcaatttacaccggtggtcacgattacgcatggtggcgcggtgcactgattgacgggattggttt
actacagggttgagttgacccacaaacactttcaggaaacggtacagacttcctgaataaatcaaattgtcacctgcggaaaaggaataatcatcagatgtat
gcccgcgagtatcgctcaacacgcccgcataaagcgattttctttcatctttcttgcctcacccttatctgtagtgcgcaagtttatgcggagccggatatgc
ggccactggggccgaatatagccgataaaggctccgtgttttaccatttcagcaccacctctttcgactctgtcgatggcacacgccattatcgggtatggac
ggccgtgccgaatacaaccgcaccggcatcgggttacccgattttatatatgcttgacggtaacgcagttatggaccgcctggatgacgaactgctcaaacaa
ttgtcagaaaaaacaccgccagtgatcgtggctgtcgggtatcagaccaacctccctttcgatctcaacagcagggcttacgactatacgccagcagcagaaa
gcagaaaaacagatctccactcagggcgttttagccgtaagagtggtggcagcaacaacttccgccagttactggaaacgcgtattgccccaaaagtggaaca
gggactgaatatcgatcggcaacgccgcggcttatgggggcactcctacggcggcctcttcgtgctggattcctggctgtcctcctcttacttccggtcgtac
tacagtgccagcccgtcgttgggcagaggttatgatgctttgctaagccgcgttacggcggttgagcctctgcaattctgcgccaaacacctggcgataatgg
aaggctcggcgacacagggtgataaccgggaaacgcatgctgtcggggtgctgtcgaaaattcataccaccctcactatactgaaagataaaggcgtcaatgt
cgtattttgggatttccccaacttgggacacgggccgatgttcaatgcctcctttcgccaggcactgttagatatcagtggtgaaaacgcaaattacacagca
ggttgtcatgagttaagccactaaacactgcccgcttttacgcgggcagtacgcctgaaacactacgatcagaatgatgcggtaactccggcatagtaagccc
ggcctggctcgttataggtattcgccccttcagaagatcggaagatctgtttattgaggatattactgacgccgacattaagacgcagatttttattaatatc
gtaattgaagttcgtccccaccagtgaataagcgcccagctctttacctgacagaccgccagtatcttcactgcgggtttccgcatgagtacgcggtttttgt
ctgccatataacgtccagttgacgctggcagaaaacgcctgggtgatggtccagttaagcgagttattgatagtatatttcgggatgaccgacagaggattac
cggtgtctttttgctccgaagtgatcatccatgtggcattggtattccagttcagacgatctttcaccagtgggaaagacatactggcttcgataccgtccac
cagagctttcccgccattctgccacttgaggatatatgcgcctgaagcggtttgcccgataacgttatccccggccacgatcttattctggtaatcattgcgg
aagtaggtcacacttgcgtggtaatcttcccaggtgaactccagcccaatttctttattgacgctgatttccggatcgagatctttattaccgatcaggtagc
acccgcctgatgtaatatcttttggacagccattgcctttcgagtagagcagatagccttcactggattgatacaggtttggggctttaaaggttcgggcaac
ccctgctttgactttgaaataatcgcccaattcctgcgaaagattcagactggggctgaagttcccgccggagtcgctgagataatcaaagcgcaggccggga
atgatattcgtgccaggaaccggctcaatgttatcttcaatatacagcgcactgatttgagaatgatttttactgctgcgatccgcagcagagccagaaatac
cgctgatatcactgtcattcaccgtcaggctggtagaggaaggatcatcgagcttatcgcggttccactctgcaccaacggtcagcgtttgatcaaccatcac
attcaaaggaatattaagctcgccgctggttcgccaggaactcaggcgattggtcgtaaacttttcacccgctaaaatacgcccttcaccaccgccggataat
ccttcattcatgcgggtattattggttttctcgtaataaacaccaaagcgactttgtccccagtcccagataccattatgcgtaatgccataattctgtcggt
acaggcggttcgtctctttgccggattttgccaggctttcggtaactgcactggaagaactgttttgcgtatcgcccgcatagatattcccctggcggctata
tccggcttcgaaatcgagaatctgctgcggatttaatttccacgagacaacgccgttaatatctttgttacgtaccccttcatgcccggctgcgtttttcgta
ccgaccggagaattaatatcccaactgtcagcatccgttttattcaggttaccatacaaacgcgtggtaagagcattaccagccagaggcccactaaggctga
aattggcgcgacgcgtagcgccctcatcgctactttccggctgattggtgtataacgacagcgaaccgtgccagtcgttggtgggacgtttggtaatgatgtt
caccacccccccggctgcccccgaaccgtagcgcgccgccgcagggccgcggatcacttcaatacgctcaacctgttccggtggcacccagttggtgtcaccg
cgggtatcacgctctccacgccagctataacgcacggagttacgtgacgtcaccggtacaccatcaattaaaattaaggtgttttccggccccataccacgaa
tatcgatctggcggttgttaccgcgtgtgcccgaggcgctattgccggtaagattgacgccaggcattttacgaataatatctgaaaggtcgtttaccggagg
ggtctttttaatatcctcgctggtaataaccgacacgcccggctgctgttttaatacctgctcagcggtggcttccaccaccagagtctcgtcattatcatcg
tcggaggatttggctactgatacctggctattcaacccaaccaggagcacagttagcgaccagaggattttgttaattctcatacctattccctaataaatgc
ctaacttaaaatgtttgatcgttaagctcacatccttgccagatatttcttactgccattattgtttttatataagaatgataattaatatcatttagtaaaa
gaaaaagcaatccctcacaagataaatatattgatttttcataaatatcaaattgatatataacatatgttttttatttcattgcacttgtcgatcatcggga
attttgtcacaatttctcacggatagtgttcacattgtttctgacctgcatttcaggcgcgggcgctgcttatgtatataagggcctgtggtcgacgaatcag
cataatcagatcttcctgcgaacaatttctttcagcgtatcaggcagcactatcggattatctacgagcgtttttgattcacattgtgtgtgaaacttcatac
atcatgattcgctttccctactcagttctgatgattgttttttattctattaacacggagttaagattctaccctcggtgtcctcattattaatgaggaatgc
tacctatctaaaaactgaccgaaactttggggcatccctggaggtttccaaggagaaaaaggtgacagtgatagtgccacattgtgaggaaattcctgagaaa
ctcctgagaaactcctggttcacacacataattcaggagcttgccatagatagtaaccaattgttttatattttaagaccattaaatacgcggctacgatagc
gctttatataatcataaactgtagagataaaatatctgcttctctgataacactaattgagtgtcttaaactaagtaatttcatttttccccctttaaaagca
atatatgtgcaatagttgaagttaaagctgtgctgccagagttgataataattgttctgtctgttgccatgatatgcattcatcagtaacagactgaccatac
tctagcggaaaactatcggattgttttccatcttgtaaaaagctttccaccataatgccagctacatatcctggtatttttttccgattatctataacttgac
gtgcaacggaaatttgccgtttagccactttaccgctattaccatggctacaatcaattatcagacgatggttaatgccctcatcatgcattaatttcactgc
cttagttatatcagacaaaccatagttaggttctctacctccacgtaatattagatgtccatgtggattgccatcggttaatagtgtagatatactatttgtt
aaagatgtcatatatacaatatgttgttcacgggctgcaataatagcgtcaatagctaagttaatatttccatcagtactgtttttaaatccaactggacaat
gtaaaccagacgcaagttgacgatgcgtttgtgattcagtggttctggcaccaatagcaccccaacatattaaatcagcaatatagggagtaagaaaaggatc
gaggaattctgtagcggttgcgacacgcatcgttgtgattgaagacaaacactggcgagcatatcgtatccctttctcaacatcataacttccatttagatca
ggatcgtgcattattcccttccagcctttacgggttcgaggtttctcaaaataagtgcgcattacaatgtacattttcgataagtacttattctgtaatacga
acaatcgcttcgcataatcaactgcggcctgaacgtcatggattgagcatggaccaacaatcacaagtaagcgaggatcttttcccagcaaaatattcgctat
aatatctcgttgcagagagatccatgttacggtttcttccgatactgcgatttctttatgaatctcgcccacagtgggtaaactgccaagcaacttcccgcgg
tagattaatttactagctgactgcatcttgtctcctaaaagcgctatttcagaccatcagttgcacacagtagatgataatgataatcatttactgtataatt
ggcaatatatggggattcatagtcatttctgagataatatacgttaggccttctctcccgtacggaaatcacttgttttccgtaaattacctgaatcttaatc
cctgagctgcggctagctgtggaactcacaaaagctgaccgggccgtggcacagcctctctgctggcagaacagcgtactgcagaagtcgtcgtactgccgct
ggtcccggcatcaggtctgcccgcagatggcacttctgccactgatctgccggtggagctgaacggcatgacatatcaggcctgccgtggggattttgtggtg
cgcctcgacggcagcacctgtctgcagttatagaataaagaaggcggggaggtacgccgggagggcgatccgctggaagtggcgtagtggctgtaggcctgta
atgatgcaggaatagaagttcgtgtacaggttaatgaaagcatcaccccgtaaaaacatgccggcacggtttacacatcattccatggtggcagccggttaac
tttcctgcgccaccagtggaaagaggccatgagcagtccgaacaaaaaaccactcgtgatacttataataatggcctcacctgctaccattcctgacggcccc
cagtacataaaccacattgcacatccccaggaaataccccataagccccccatcagcagcgtaacctgccagaacggcataaagggcaatggcggaagccgga
tacccagccgccagagaatacgcagcagaggaggagcataattactccgccacatctttttgctgtccatcagggcaattgcccgggctttttttgctcaaaa
gtcacaggagcacctccgttccatgatgtaaacgtgacacaacttattgttattgattattattatcataccgtgattatgttgtcatgcacctgtacactga
taaaagaaagggagaacaggaggctggctgaacaaccaatccgttcgtccagttcaatggcggctatcaggttctgctccgcaatgtactcacgaatgcgctt
cctgtcctggctggccagcatggtccagaatatctccatcatttaccggcgactttcctgctcatttcatcccgccatgcagcagcggaagattcgaccatgt
catggggaagcacatcgccgcgctcgagttgctgacgtcctgcctcaacctggtcacggaaccaggtattatgctgccgttccacggtctggcgcataaaatc
ccggattaactgagagccattacggtccatgctttttgcagggccataaaagcatccttcagttcagcgtcaattcttaaactcatattaacctgtgccattt
atcaccccgtcattaccacgtatatacacagtatataacgattcagatatcgtcactaggatatgccgcgccagcggcatggaaggcggcactccgctgtttc
atatgataccgccgaagcccgatgtaagccgctacagtcgtccgaaagtcaccagcctcctcccccctgccgtcatccgtgcatcagctatgcactgagtatg
cctgcccttccctagagaatcctgccaggcttgccacactgatatatcttgactttatgtaaacgatatgacactttaacatgataatgattaccattctctt
ttaatatacagagaaactaggaaatagatgaatgagttatgttactttaatattctctgacaataacctaaatcagttagattattgtcatttaataaataat
gacattctttcatcataaataaaaagactattgtttataatattgttctcagcattatatgattatttatcctgataactctcctatgttgtatgtttatatg
attttccttgaaacatataatgcaaattttcgatttattttccatcattaatccagataaacaacaaactaatagtatgcaaggagacattatttgtttcgcc
aggatgctttagaaaacagaaaaatgaagtggcagggacgggcaatattacttcccggaataccactgtggttaatcatgctgggaagcattgtgtttattac
ggcatttctgatgttcattattgttggtacctatagccgccgtgttaatgtcagtggtgaggtcacaacctggccaagagctgtcaatatatattcaggtgta
cagggatttgttgtcaggcagtttgttcatgaagggcagttgataaaaaaaggggatcctgtttatctgattgacatcagtaaaagtacacgcaatggtattg
tcactgataatcatcgccgggatatagaaaaccagctggttcgtgtgaacaacattatttcccgtctggaagaaagtaaaaaaataacgctagataccctgga
aaaacaacgtctgcaatacacagatgcgttccgtcgctcatcagacattatacagcgtgcagaggaagggataaaaataatgaaaaataatatggagaattac
agatactatcagtcaaaaggactaattaataaagatcaattaactaaccaagttgcattatattatcaacaacaaaacaaccttctcagtctgagcggacaaa
atgaacaaaatgccctgcagataaccactctggagagtcagattcagactcaggcagcagattttgataatcgtatctatcagatggaactgcaacgactcga
attgcagaaagaactggttaacactgatgtggaaggcgaaatcattatccgggcgttgtctgacgggaaagttgactccctgagtgtcactgtagggcaaatg
gtcaataccggagacagccttctgcaggttattcctgagaacattgaaaactattatcttattctctgggtcccgaatgatgctgttccttatatttcggctg
gtgacaaagtgaatattcgttatgaagccttcccctcagaaaaatttgggcagttctctgctacggttaaaactatatccaggactcctgcgtcaacacagga
aatgttgacctataagggcgcacctcaaaatacgccgggtgcctctgttccctggtataaagtcattgcgacgcctgaaaagcagataatcaggtatgacgaa
aaatacctccctctggaaaatggaatgaaagccgaaagtacactatttctggaaaaaaggcgtatttaccagtggatgctttctcctttctatgacatgaaac
acagtgcaacaggaccgatcaatgactaacaggaatttcagacaaattataaatctgcttgatttgcgctggcaacgtcgtgttccggttattcatcagacgg
agaccgctgaatgtggactggcctgcctagcaatgatatgcggtcattttggtaagaatattgacctgatatatcttcgccggaagtttaatctctctgcccg
tggagcaacccttgcaggaatcaatggaatagcggagcaactggggatggccacccgggctctttcactggagttggatgaacttcgagtcctcaaaacgccg
tgtattctccactgggatttcagtcacttcgtcgttctggtcagcgtaaagcgtaaccgttatgtactgcatgatccggccaggggcataagatatatcagcc
gggaggaaatgagccgatattttacaggcgttgcacttgaggtctggcccggaagtgaattccagtcggaaaccctgcagacccgcataagtcttcgttcact
gattaacagtatttacggtattaaaagaacgctggcgaaaattttctgtctgtcagttgtaattgaagcaatcaatctgctaatgccggtggggacacagctg
gttatggatcatgctattcctgcgggggacagagggctactgacgctaatttctgctgctcttatgttttttatattactcaaagctgcaacgagtacgctgc
gcgcatggtcttcactggttatgagcacgctcatcaatgtacagtggcagtcggggctgttcgatcatcttctcagactaccgctggcgttttttgaacgccg
aaaattaggtgatatccagtcacgttttgactcccttgacacattgagggccacatttaccaccagtgtgatcgggttcataatggacagcattatggttgtc
ggtgtttgtgtgatgatgctgttatacggaggatatctcacctggatagttctctgctttaccacaatttacatttttattcgactggtgacatacggcaatt
accgacagatatcagaagaatgtcttgtcagggaggcccgtgccgcctcctattttatggaaacattatatggtattgccacggtaaaaatccaggggatggt
cggaattcggggagcacactggcttaatatgaaaatagatgcgataaattcgggtattaagctaaccaggatggatttgctcttcggaggaataaataccttt
gttaccgcctgtgatcagattgtaattttatggctgggagcaggccttgtgatcgataatcagatgacaataggaatgtttgtagcgtttagttcttttcgtg
ggcagttttcggaaagagttgcctctctgaccagttttcttcttcagctaagaataatgagtctgcacaatgagcgcattgcagatattgcattacatgaaaa
ggaggaaaagaaacctgaaattgaaatcgttgctgatatggggccaatatccctggaaaccaatggtttaagctatcgttatgacagtcagtcagcaccgata
ttcagtgctctgagtttatctgtagctccgggggaaagtgtggctataactggtgcttccggtgcgggaaaaaccacattaatgaaagtactatgtggactat
ttgaacctgatagcgggagggtactgataaatggtatagatatacgccaaattggaataaataattatcaccggatgatagcctgtgttatgcaggatgaccg
gctattttcaggctcaattcgtgaaaatatctgtggttttgcagaggaaatggatgaagagtggatggtagaatgtgccagagcaagtcatattcatgatgtt
ataatgaatatgccaatgggatatgaaacattaataggtgaacttggggaaggtctttctggcggtcaaaaacagcgtatatttattgcacgagccttatacc
ggaaaccaggaatattatttatggatgaggcaaccagtgctcttgattcagagagtgaacatttcgtgaatgttgccataaaaaacatgaatatcaccagggt
aattattgcacacagagaaacaacgttgagaactgttgatagagttatttctatttaaaccatagaggaattacaagcgtatgaggaatatttcttcctgtta
taattcctcgttatgctcagatatctgttggaggtggaatggaagatagacaatccaccaagaagaaatatcattctgtgtggattgtccaataactgttctt
tcttatattaaataatactatttataaacaaacatcactaagattatttggactccaattacacaatcttcccgcagcatagttccatgcttctgaaggtatc
ccttcgggtttttgcttaattgttccccctaaaccggatggagacattgcaggattaggtttgtgagtggatgcatagtcatatattgcacctccagccacac
ccccagcagctgctccaattcctcctgcaacaaattgcccggatagtgttcctatagccatcgcaatatcacgccctgaagcaccaccagaaacagaatctaa
ttcatttagagtcagagttctcatatgatctcctttttatcttatcggatattgaataataattatcaccaacaaagtaacatattgcagacattaatgcaga
gaagcaaaatgtatgcatggataaaaagtcctttcctctaaaaacacaatcatatatagctaatgcaatatatattgcggtggcatttattataaatgcaaat
aacaactctaattttgttctttttctatccattactttttatcccattactttctatcccattaccacacaaacactaacgataatgattatcgttaacatag
tcaagagtgaagggtaggaggccctcaaccccctataaggggtccgcttggaaaacggatttccccacgtcaagagaattgatttgaacgagtggcttcgcta
caaacagctcccgtttgtgtgtgaaaaacccctctcaacagatctcaactctgcacgatatctacaggagattgctccaaaaggcagtatcgtgtcttttaac
taccgatataaccccgtcatcgacatgataatgcgtctgagaaatgagaagaaattaggtgaactgcatttcttctcagcagaatttaataaaaattctgcac
tgacccgccatcacctgacctggcgagactccgcacaacagagcaaaagcagcggtgctctgggcgatctatcctgtcatctgcttgatttattttgttttat
aggggaaagcccggtagtggttcatgggatcaaaacggtaaaggggacacgtgggtacaaaatcagatggtcaggttgaagtggatgataacggctatgtaat
gggcagttcagaaaagggagcttatttcagagttcatgccagtaaatcggaaacagatcataatttgggactgcatatacagcttgtatttgaaaatggtgaa
atcagatattcaacccaccatgaaaatcgcctgctcctgattctatttaatgatacgaatacggaaacaattggcttcgacgctctgaaacgcttgcctgatc
caccacgggagcttccattctggtctgattcgtttattcatcttcatgacgactggtgtgctcttattaaatatggtcactcgtctcctaagctagcggacct
gtcatccggccttcatatacaggaaataattgaggcattctgatatggataccattcttttaaccgggctgtttgctgcatttttcacaacgtttgcgttcgc
tccccagagcataaaaactatcaggacacgcaataccgaaggtatctccgttgttatgtatatcatgttcctgacaggtgtaatctcatggatagcgtatggc
attatgcgatctgattttgccgtactgatcgccaatattgtcacactgtttctggctgcaccagtgcttgtaataacacttatcaaccgcaggaaaaaacacg
tatgaatattgcaatcatcggcgcgggtcctgccggcatcatttctgcccgtaatgcgattaaagcaggtcactccgtggttctgtttgagaagaatacccga
attggggagatctggaacccctggagtggtggcgcttaccgtaatgcctgtatgcagaactctcgttatacatttcattatactggctttccccctggcgata
tcgatgaatttctaggagtggaacaggtattcagatatctttcggccgtggccggagaggatgccctccgtgagtcgatccgactgaatactgaagttgtttc
actcagaaaagacgccggacaccgggtgatccgctgcgcttctgaaggaaaagacacggaagacatttttgaccgggtcatcattgcaacaggtgaactctgg
caaccccgccggccccccctgccaggtgaggaaaacttctccggaacgttgatcacgtcgagagattatcaggagccagaggcatttaaaggaaaaaatatcc
tcatcattggcggcggtgtcagcggtgcggatattgcctcagaccttgttccctttgccagaagcgtaagtctgtccgtcaaaaagatgggactttatctgcc
gagacaattcccgactggcccgaatgacatgatgcactcctatctgggcaggtgtctgctgagccaaatgaattatgaagattttatcggttatctcgacacc
atgatgcctgactacatgcaggcctaccgagcatccagtctgttgcccgacatggcgaacaataacgcggtgcatgttaacgaaaaaatcataccgaatgtgg
ccgccggtctgataaaagttaaaccccaggcagagcgtttcaccggagaaggcgctattaagttcgtcgacgcatctcaggaaaaatatgatgtaataataac
ctgtacaggctatgaaatgcctgattactcgttcattcgtaagcgtacagcctgaaccgtctggtcagaatctgacgaattagacaaagtggtgtccaccaaa
taagtagtgggaaccaaagtgtcagatatgcagaaaaatgtgactcccggcaggcgaaaaggctgccctaattatcctcccgaatttaaacagctgctcgttg
ctgcctcctgtgaacccgggatatccatctcaaaacttgctcttgaaaatggcattaacgccaatctgttgttcaaatggcgacaacaatggcgcgagggaaa
gctgctattaccttcttcagagagcccccagctacttcctgtgactctcgatgcagctgccgaacagccagaatcgcttgcagaggacccggaaaccctcagt
atcagctgtgaggtaacgttccggcacgggacgctccgcttcaatggcaatgtcagcgaaaagctcctgactctgctgatacaggaactgaagcgatgatccc
gttaccttccgggaccaaaatttggctggttgccggtatcaccgatatgagaaatggcttcaacggcctggctgcgaaagtacagacggcgctgaaagacgat
cccatgtccggccatgttttcattttccggggccgcagcggcagtcaggttaaactgctgtggtccaccggtgacgggctgtgcctcctgaccaaacggctgg
agcgtgggcgcttcgcctggccgtcagcccgtgatggcaaagtgttccttacgcaggcgcagctggcgatgctgctggaaggtatcgactggcgacagcccaa
gcggctgctgacctccctgaccatgctgtaaatctctttatcctggttgtcacagaataagcccggtaaaatacgggcttatgaacgacatctcttctgacga
catcttcctgctgaaacagcgcctggccgaacaggaagcgctgatccacgccctgcaggaaaagctgagcaaccgggagcgcgaaatagaccatctgcaggcg
cagctggataaactccgccggatgaacttcggcagtcgttccgaaaaagtctcccgccgtatcgcacaaatggaagccgatctgaaccggcttcagaaagaga
gcgatacgctgactggtagggtgtatgacccggcagtacagcgtccgttgcgtcagacccgcacccgtaagccgttccctgaatcactaccccgtgacgaaaa
gcgactgttgcctgcggcgccgtgctgcccgaactgcggcggttcactgagctatctgggcgaggataccgccgaacagctggagttgatgcgtagcgccttc
cgggttatccggacggtacgggaaaaacatgcctgtactcagtgcgatgccatcgtgcaggcacctgcaccttcgcggcccatcgagcggggtatcgccggac
cggggctgctggcccgcgtgctgacctcgaagtatgcagagcacaccccgctgtatcgccagtcagaaatatacggccggcaaggtgtggagctgagccgttc
actgctgtcgggctgggtggatgcatgctgctggctgctgtctccgctggaagaggcgcttcatggctatgtcatgactgacggcaaactccatgccgatgat
accccggtccaggtactgctgccgggtaataagaagacgaagaccgggcggttgtgggcgtatgttcgtgatgaccgcaatgccgggtcagcgttggcacctg
cagtgtggttcgcttacagcccggacaggaaaggcatccatccgcagactcatcttgcttgcttcagcggtgtgctgcaagcggatgcgtacgccgggttcaa
cgagctgtatcgcaatggtcggataacggaagctgcctgctgggctcatgcccgccgaaagatccacgatgtgcacgtccgcatcccgtcagcactgacggaa
gaagccctggagcagatcggtcagttgtacgccatagaggcggatataaggggaatgccggcagagcagcggcttgctgaacgtcagcgaaaaacgaaaccgc
tgttgaaatccctggaaagctggttgcgtgaaaagatgaagaccctgtcgcgacactcagagttggcgaaggcgttcgcgtacgcacttaaccagtggccggc
actgacgtactatgcgaacgatggctgggtggaaatcgataacaacatcgctgaaaatgccctgcgggcggtcagtctgggtcgtaaaaacttcctgttcttc
ggctctgaccatggtggtggcggggagcgctactgtacagcctgatcgggacgtgcaaactgaatgacgtggatccagaaagctaccttcgccatgtgcttgg
cgtcatagcagactggccggtcaaccgggtcagcgaactgcttccgtggcgcatagcactgccagctgaataacacatccccgtcaatacggccctcgctgta
cgcttaccaataaataatatgcttatcgacctgatggctgcaatgtcgcataaagactggctgagtcgtcgtcaacgacaaaaacagggaattgaacgtgccc
atactttaggtaagtatcggggcaaacaggcagatcgggaacgacatcagaaagtaatgtactaccggcagatcaagaaactgagcatccgtgaaacggcaga
ggtgacaggctatagtacttcccaagtttgtcgcattcaggcgctctataaagaggttaaacctgattaaaaaatagccttaacgtacaatattttccgtttc
ccaaacggagcccaatacagatggctaacagaccgtctgcttccagttcaaatggtttaccgtcgcggtccagggcgcgaattttaactttctctgcgcgtgg
cactaaagctcgttgttcagggattgacaggcaaccttcttcaatgcctgtttcgccgcttttttctaaaagctccggattgattaacaccagccgttcgtca
cggttttccgaaacatcaattacaatgatacgttgatggatatcaacctgggttgccgccaggccaatacctttttctgcgtacatcgtctcgaacatatcat
cgacgatacgctgaatttctgcattcacttcttctaccggtttagcaactttgcgaagccgctcgtccggaatatgtaacacttgcaaaactgacataaatct
ccagagatgtgttcaggagttagaaagattatttcttctactgtaccgtaatcagataatcgcagcaaagggacaaagcaatagtccctttgctgcacagcat
cagaatgaataacgaatattagcgtttatcgcatcatctgtgttgtatttaccaaatgcagagcgttcaacttccagccccaaacgcgtattgtcgccaaacc
gggcatttaaccccacaccgtaaagcatacgactgtcttttctgccattaatctgatgttctcccgctgcatccttcagatgaacgtcagcactgtccgtcag
atcgaactcataatgcaggccggcacgggctgtcagactccagtccttaccactgaaggttttaccggaaacaacgccggttctgcctaccagaggattaacg
ctgttacgacgcattgagacatccattccactgtcgttccagttaaatgtttggccctgcagtcttccccagaccagttccgcctgaggttcaacaaacgtcg
tatctgtcagatgataacggtatccgacttctgcacctgcatacagtgaatggctgcggaagttctgtttaccagctccggcaaagttcaggtcatatttgtt
ttcattgtgaatatatttggcaatcacatcaaagtaagcgccggaccggaacagaccactggcatagaaaccaccaccccatgattttgttttaccgctgtac
aggtctgctgacgcatctgtgtcagtgtaggtggccatcacgccggtaaacaggtccatacttcccagttcgtgcttacggtcagcccccatctgcagcaggg
tatagtggtcagtgaaaccgccatcagcagagccggaaccgttcagcagacgcacccacgtaccggcttcgccattaatatccctcaaatcgcccatgcgttt
gttcaggttgttaacttcagtgatgaagttgttatagctgatgtgcatgaatgtggcggcagccttaccctggccgtcgttacgtgcaacctggtaaccatcg
aggacccactcttttttcccgtcctcttttctgacactaaggatgggggtgacatcactgaatcccacaacccgtgttgatgccctgaacagattatcagctg
tcgcttcaggtgcgctgaccagtggaatatcaagcgtgtccttgttagaaggttttttcaggaagttaacccagatgctgttgtcatgacctgttgccgactt
gtttatcaccagtttgtctgccttgttaaggtctgtacgcatgacaaatgctgactgaaccgcgtccagattatctgttgtcagtgtcgtgaacggtgatgtt
cccccgttaaaaccgactattgtccggttaagtttcatatttcctgccgtggagtttccgttcatcgaccactgggtgtctgtcatgctgacggtggcatccg
gtgcattcaggctcccgctccagatattgcggtacccgttaaacaggctgtacaacatctgattctgaagagtcaggtcaggactcagttccccttcccctcc
gagggtgacagtccctttatcctgaacattgatattacctgacaacatactgtacggccccacgttcaggcgggcatcgtctcccttcaggttccatgaaccg
gcataatcgtatacaggtaaaagtgtgtgagatacctcatcaggacggctgttcagactcagggcggcatcagaaatattcactggaccgtcagaaacaaaac
tctgactggccagagcatttgctcccttgttcagattcagggcggtactggtcagtgttgagttccccagaacggcactgtctgaggagatattcacggtact
gttgttcgcctgtattccgccattgaatatatcattgatattcagcactgactgattatccaggttgacggtgccgttgaagacacttttatctgcatcttta
gttgcaacagatgtgccttcttcaagggtaaaggctgttccctggccatcgtttttgtcgataaatacccggctgtcgcccagcgtgacgctggagttatctg
cctggatggttgtgttcagtgtggcattgcgaccaagaccaaaatcagtgttcttcagtgacagcctgtcaaaggtaaaactgcggttctcccagtcctcctg
actgaatgacgtaggctgagtcagaaccgaatggtctccactggcagccagtttgtcagccacagactgagtattgtatgcatggataaccggatgcccctgc
agcgtcagacgcccgttttcctgggtgaatgtaccggagatatccgcagctccgtccatcaccagagcaccggtaacgccttcaggcaggcgattgtccacat
tcagattgcctttcagttgtccgtgaaacagataccctgcagtattgaaacggtcagctaccagtttctgtgcatccccctgactgtgccccacaaactccca
ggtggcgttgctgctctggtccgtggggaaataaccataggtgctctgcttcaggatgaagtaatccgtcgtatttgtgtacgggttattgtatttatataaa
tttccggcagttcctttacctgattccgaccagccattcagtgctactttgtcagcccgcagggcatagtccagcgtgatagtggcccgtttatcaacgttat
tcgccagcacggcaccataatctgccgccttcaactgatgaaacgtcagactgttaccattaacatccagtgtgcccccacgatatccccatgagacggtatc
cggatttacctgccgctcatcagtcagtaccacggtcggccggccactggcaatattaacactgctgaacgcctgcacctgtcctttattgtccgcctgctgg
ttcagtacaacctttccgtccccgaccttcaggccaccttcattaatacctgtaccctgtaccgtcagcgtaccttcaccaattttatgcaggttatcgccct
taacaccattaacctgccagtttacggacaccccgttgtccacaacaataccggcaccggtccaggtacttccgttagaggttgttactgtgtaattatcacg
gaacgtcagggaacccgccccctgagaaaccgaatccttaaggttaatctgaccattctgcccctgaaatatcaggttcttaccagcattcaggtcatttccc
tgctgcccgtgcatggcatatgtggtggttccctgtgtcagcgcaccagctccggtactgctgttaaagctccactccagtgcaccaccttccgatgttctga
acgtgaccggggcatcattgtcttcattaaatttctgcccgataaaatccagtggaataacagcccagttatttcccctgccccccgcgccattccccgcagt
aagaacaccgaccagcacccatttattctgaacagtatcaaaagcaaataaaggcgaaccgctgtcaccggcctcgccataaatgggcattgcaccattaagt
ttgtaatcaaaaaccagacctgaactggtgctaatcatttctccattctgataggatgacaggctaccgacagtcccgccggtcagccaggaatatgcacctc
ccatttgtgtcagctgtccgttactgtccttaatatactgagtaccagaccccagacgataaaaaacaggatagcgctccttatccagatatgcgccagccac
tgccccctgcgccgtcaccgcagtaggggcaacctctgtcaccagtttatccagccggggggcatgaaaatccagtgacggcgcattattccggtccacgata
ttgtaacggttttcaccatcaccaaagctaacgtttgtatatcccccgttatgtttcacgctggcgatatactgcgggtttatcagtgtcgccacaccgattt
ccgaatccacagcactgaaatcaggcatagctgccttatccagcgtaccgacaaattctccctgcttattataaatagcgatattcgttgcccccgggcggaa
catccccttattttcagcaaaatcacgaaataactgatacccgagttcattattgaccgttcccgcaagacttcctgcagagaatagtaccgggatcagtaat
aaaaccgggaaacacagacgtctgacagacttatgaacacatttcctggcaaactcagatacggcaataaagccccgggccacagcgctgtagcgaagagaat
aaattctgttcatttttaattactcctgagaaaaataaaatcagtactactcaggcataacggataatcaccatcatcctgaggaatccccagaaaaaggaga
caggcatagcgtgatgtgaactactgattctttcacaaattcagggagagcaccgttatgcctgcacataaagtatgccagaaccggtggcgaatgtgtgcta
tgcacaaatggtgaaacagttcctgagtcgtgacccgctcgaatgcgtgctgtgtggcggccgaatggtataccggcgtgccatagcggggctgaatgtggac
gggctgaagaaaaacgcgcgggatatcagtctgataaggattactttccagccggctgatggctgacgggcgtaatcccattcgttcggccagcgcagacttt
gttaaccccgctttttctctcatttcgtataacatttctaccatctccagttctttgtcagcatcttcataccctttaacagcttctggagtattaagcagtt
tttgctttacctggctgaacggaacactttttactttcgtcatttcatctccttcaggcgcatccttgaagtggtttactgaatttggccacctgaacagagg
tgatatgctcacctcagaacaacacaggtgctccaatgaaaaaaagaaatttcagcgcagagtttaaacgcgaatccgctcaactggttgttgaccagaacta
cacggtggcagatgccgccaaagctatggatgttggcctttccacaatgacaagatgggtcaaacaactgcgtgatgagcgtcagggcaaaacaccaaaagcc
tctccgataacaccagaacaaatcgaaatacgtgagctgaggaaaaagctacaacgcattgaaatggagaatgaaatattaaaaaaggctactgtagattcaa
ttggtcaacgcaacagttatgtgaaaacatggggttgcggaggttttttgaatgagacgaacatttacagcagaggaaaaagcctctgtttttgaactatgga
agaacggaacaggcttcagtgaaatagcgaatatcctgggttcaaaacccggaacgatcttcactatgttaagggatactggcggcataaaaccccatgagcg
taagcgggctgtagctcacctgacactgtctgagcgcgaggagatacgagctggtttgtcagccaaaatgagcattcgtgcgatagctactgcgctgaatcgc
agtccttcgacgatctcacgtgaagttcagcgtaatcggggcagacgctattacaaagctgttgatgctaataaccgagccaacagaatggcgaaaaggccaa
aaccgtgcttactggatcaaaatttaccattgcgaaagcttgttctggaaaagctggagatgaaatggtctccagagcaaatatcaggatggttaaggcgaac
aaaaccacgtcaaaaaacgctgcgaatatcacctgagacaatttataaaacgctgtactttcgtagccgtgaagcgctacaccacctgaatatacagcatctg
cgacggtcgcatagccttcgccatggcaggcgtcatacccgcaaaggcgaaagaggtacgattaacatagtgaacggaacaccaattcacgaacgttcccgaa
atatcgataacagacgctctctggggcattgggagggcgatttagtctcaggtacaaaaaactctcatatagccacacttgtagaccgaaaatcacgttatac
gatcatcctcagactcaggggcaaagattctgtctcagtaaatcaggctcttaccgacaaattcctgagtttaccgtcagaactcagaaaatcactgacatgg
gacagaggaatggaactgaccagacatctagaatttactgtcagcaccggcgttaaagtttacttctgcgatcctcagagtccttggcagcggggaacaaatg
agaacacaaatgggctaattcggcagtactttcctaaaaagacatgtcttgcccaatatactcaacatgaactagatctggttgctgctcagctaaacaacag
accgagaaagacactgaagttcaaaatgtcaacgacggatgaaaagtgatccacttatatctccaccaacggcccaatattgatccaccgttttactcaggat
tagcttctgctataaccccggcctttcgtttctgtctgagtcgatagctttctcctttgatttgaacgacatgtgagtggtgtaagatacggtccagcatcgc
tgaggtcagtgctgcatcaccggcgaacgtttgatcccactgcccgaacggcagattggatgtcaggatcattgcgctcttttcgtaacgtttagcgatgacc
tggaagaacagctttgcttcttcctgactgaacggcagatagcctatttcatcaatgatgagcaggggggggccattactccacgctgaagcgtcgttttata
acggccctgacgttgtgccgtagataactgaagtaacagatctgctgctgttgtgaagcgaactttgatacctgcacggactgcttcatagcccatcgctatt
gccagatgggttttccccacacctgatggccccagtaatacgatattttcattacgttctatgaagctgagtgagcgtaacgactggagttgcttctgcggtg
ctccggtggcgaatgtgaagtcatactcttcgaacgttttcaccgccgggaaggctgccattcgggtatacatcgcctgtttacgttgatgacgtgccagttt
ttcttcatgaagcagatgctccaggaagtccatataactccattcctggtctactgcctgttgtgacagcgcaggcgctgcgcttataaggctttccagttgc
aactgcccggcgagcaccatcagtcgttgatgttgcagttccatcatcacgccactcctctgcagaatgagtcgtagatggagagtggatgatgcagggggtg
tttgtcgaagttcaccagattttcatcaagatgcacgtcatactcttttttctccggaggcagtgccagcatggactgctgctcttcgagccagcgatcgcag
ggacgtgcctggattgtttcatgctttcgttggttagcgacatcgtgcagccagcgcagaccgtggcggttggctgtttcaacatcgacagtgatccccatcg
ggcgcaggcgagtcattagtgggatgtaaaaactgttacgggtgtactgcaccatccgttccaccttacctttagtctgtgccctgaaggggcgacacagtcg
gggagagaagcccatctccttgccgaactgccacagcgaaggatggaaccggtgctgaccggtctgatatgcgtcacgttgcagaaccacagttttcatattg
tcatacaacacttcgcgcggcacaccaccaaagaagcggaacgcattacgatggcaggtctccagcgtgtcataacgcatattgtcagtgaattcgatgtaca
gcattcggctgtatccgagaacagcaacgaacacgtgaagcggtgagcgaccattacgcatagtgccccagtcaacctgcatctgtcgtccgggttcagtttc
gaaccgaacggcaggctcctgctcctgaggaaccgagagagaacgaatgaatgccctgagaatggtcattccgccacgatatccctggtctctgatctcgcga
gcgattaccgttgccgggattttgtaaggatgagcatcggcgatgcgttgacgaatataatcccggtattcatccaggagtgaagcaacagcaggtcgcggcg
tatattttggcggctcagattttgcctgcaaataacgtttaacggtattgcgggagatccccagttctctggcaatcgcccggctactcattccctgcttgtg
caggattttaatttccataactgtctcaaaagtgaccataagctctcctgaatcaggagagcagattaccccctggatctgatttcaggcgttgggtgtggat
cactattgcaccgttcgtgacacaaaacaccgaaagagataattgaaaggggtgttgcattgacagattgaatctacaaccgcgctcttgatgtcagactccc
tcgagtaggcagcctggcggctgcggcttgtcatggcctggaattaccgttataaaaaaagataatgtcattgtctttcaggtagttatatggcccgttcagc
taaaccccgtaaacgcaaacccgcaccacaaagaagcaaacttccccgctatgttgtgaaacttcatccggatgatttttttgacgaagaagacgctgaagtt
ctgcgctttgataattttgacgatgccgttgagtgctgcgctgacctgggtattccgttctttctggatgcaggaaacaaaaagctggtcttctggtttgttc
gtgtcgatgacgaagggtatccggaaatagcccgctgtacggagcgggagtttgcaaccattcttgccggtatcagtgccggtggtatgtactgcccggaatg
cggcacagttcactggccggatggcgttaccccacccgtctgatgcttccccgttttgccgatatttttcagcagggtaaccgctggcttaactggctggaga
aacagccggaaggttcagtgcgtccggtggtgactgagtcagtgacaaaaatcatggcatgcgggaccacgctgatgggctacacgcaatggtgctgttcgtc
accggactgttgccacaccaaaaagatctgcttccggtgtaaagccgctcctgtccgaactgcggggtgaaggctggcgcacagtggatacagtatctgctga
gcctggtccccgactgcccgtggcagcatattgtgttcacacttccctgccagtactggtccctggtgttccacaaccggtggttactggcagagatgaaccg
cattgcagcggatgtgatactggaaatctgccatcaggcagatgtggagccggggatattcacggtgatccacacatgggggagtgaccagcagtggcatccg
catatccatttatcgacaactgccggtggtgtgacgtcgggccacacctggaaaaatcttcatttatacgcccgtaaggtgatgagcatgtggcgctaccgga
taacgcggctactgtcccggaaatacccggagctggtaataccggatgaactggcagtggaaggaaacagcaaacgggactggaatcgcttcctggacacgca
ttaccgccgcggctggaatgtcaacatatccagggtgatggataacgccacacatgtggcggtgtacttcggctcttacctgaaaaagccaccggtgccgtca
atgagtcggctggagcattatgccggtcaggatgaaatcggtctgcgttacaacagtcaccgtacaaaacgggaagaatacctgttgatgagtggagatgagt
tggaaaggttctcctggcatgtagcagataaggggttccgtatggtgaggtactacggtttcctgagtccggtgaagcgccgcttactggaagaagttgtgta
cgtcataacggagacggtgagaaaaacggcgatgcaaatcaggtggagagggatgtatcagaggttaccgactggcagagctggtcctgatgcatgagcgact
ggcacgacagcaggtgtgcggctgagagtcgcagaggggaagttgcgtccattttaccggaaacggagcaaaaaaccgccattcataccctgtatcaatcagt
gtcatcctgtttaatagtcgtttccgctcatatggtgcacaagaggtgttgaagaaatatctgttttgtggtgcttttttagtcttttggggatttaaattcc
tatcgatcaaggtcacgcatcagctttttatatgagcaaaatccaggctgcttttttatccattcagtatctatttttaaccttattaactttttattatgtg
ttccatttatagacgtagactcagatatattaccgactcttcgcataaatgatttttctgattcacttaatttttcagtaccgttacataatccgtgaccata
tggtaatggagatgtcgttaaccatactttattatacaaaattgttccatcatacaacgttagatatccttctctcaggccttcagatgcaagtatggaatgc
aacataggaattgctgtaaagtgatataaatccataaataaccctattctgtctatttatattcattttatatcatgttcaggccagactggcgagtcgtgca
tagcctgtttactgaatgccgcttcagcggcatgtaaggcggcacgctgtggttacatgtgataccggagtaaaaccgccgaagcccggcgtaagccggtact
gattgatagatttcaccttatccatccccagccctgtcagaccatacccgctttcagccatgacagagtttctgtgcgcggtcggagtggtcccgacgagggt
ttacccgaagtcggggcgtatctccgcgttagcgggccgtcagggccgcttacgagcgtgtattcaatacttccagccagaagactgacagcgacggcaatgc
ttagttacaacattcataattaaaagcgactctgttccggcccgaagggccggggggggccgcttttcagttatgagggaggggctttgtggtttcagttctg
cgctggttcggggtttttctggaggttggttttgtgtgttgtaactaaagtggctccggttggggcccgccgtttacggtgggaggtgcatatctgtctgtcc
acaggacaggcagtgaataggtttttttttaaatgaatgtaattaagtagtttaaaggagatataaacaggtgtttaaaagatacattgcaccctgtaaggct
ggcgcctggcgctttatgacatgaacggttgtaaccttatggggaagtccattgcagttaaatgtggataagcaaaattccccgtctgtgaggcgtgttttgt
atcaaaaacaggggggggccagatgcaccagaaggtggatgatgaggttgttttttgtatgaggtgctgcttttttgttcgctggcaggcttcaggcgtgcga
atgcctccggtgcgggccggattattcagaagaggtcactttcagggggaagctgtggccagccggctgtaattacggttacgtgacagaatcatgcgctctt
tcacacgacgctccacttcgcgttttaccgcctcacgattggcagtgaagcgcccttccgagatttcacgcgtcagctgccgtttcaccagggtgacgatatc
ctgacgttccctgttcgcatcacgacgcgcacgggcacgctttattccacgggacttaagctctgtctggtaactgcggaaacgctcacgcacaaaacgccag
gctttcgctatcagttcatccatacccagggtatccagcccctgctttttgcgctgtctgttttcccattcaacacgactgcggcgcgcagcaaccactgcat
cctcagacacatcaagggcggcaaataacgccggtgtgaacgtgatatcggtcggaatgtagcacccgataagcggatcatattccgtctggtaggtaatcag
tcccagctctgcaaggaacgtcaaggctcgggaggcccgggtgatggagagttttccggcagcagactccgtcgccagtccgcactcaatggccagcgtagtg
atggagcactggacgcggttggccagcgggtcatagtgaaaacacagcccctgcagcagcgcatcaatagcccggcgacgcagtaccggtggcatgcgccgac
gcagtcccttcgaacgggcatgtgccacatgaatggcgaaatcaaaacgggaggtgaagcccaccgccttttccatcagtttttcgcagaacttcagcgttcc
ggcaccttcacggggtgtgaacaccggattcggatttttaacctgtgaatagtatttttgaaggagatcagccatgtccccctccaacactattccccctcct
acgggcggtaactagctcgtctgggccgacacgtaaagtttcgaacatatctggtctgaaccagatggttcctccatcaaaagcgatgagatgaagatcatta
tcaagagttttgagggctctccaaacacgagtgtatgaacctccactacaaactgaaagctctttaacagataagttaactttgttcgtatcagggatgtagt
gtgcgcacattacttgtacgagggaaacataaaaactaaccatcgacgcgctgattttatttttacgtttgccatgcgctattgcaagcaaacctctgatgaa
gatcagatgcttgaaggagtcaaaatgagcccttttctggagaaggctgcaaaacgggtgaggatacttgctggaatcaatgtgaagatctgattcaatccca
cccgctattactgaatgtgtttcatgattagccacaccatcctgcacttacaatgcacagaaggagtgagcacagaaagaagtcttgaacttttccgggcata
taactatactccccgcatagctgaattgttggctatgcggttttatggggccccggtaatctttctagtcgccaaacttgaagaagattatcggggttttcgc
ttttctggctcctgtaaatccacatcagaaccagttccctgccaccttacggcgtggccagccacaaaattccttaaacgatcagcaatctaccactcactcc
tgagataagcaagaatgtgaatatttacgaagtcgctctgcgtttcagctctgattcaatcagtttttcaagcatctgcgcctgagtaataccttcctcatcc
gccagttcactcagcctgtcttttaaccgggcctgaataaccgcatgaaaagccttatgagtgttgcttctgcgagccagagaagctctttgcctctctctgg
ccggaacagggttacctttccggtatgtacgcttggtgcctgatgaggaagtcactgcattttctgtctgcgacatctcgcctcctcaattctcaaacaggga
tcgtttcgcagagaatactacagttttttgaaatcagcgacttgaaagttgtgacgatgatctgtgttggcgcacaaatcatgtctgattacgaccgtttaat
gtgattttgataatgaaatgactgaaaagctggaaatgccttagccagggtgccggccccggggggaccggctgtttctggtatatagggtgtctgcccctga
aatacctcacacttccggaaagcggggacttccgtccccgcccggcagcttacttccgtgcttcgtaggccagagttacctgcaccactgtatttcctctgtg
aatattcagctcacataaccgttccctgaatatcagtgaaaaacacaacaccgtggcgcacacggcgagcaacccgataagggcatatttcgtcataaacttt
gacctctcagcaaagaagaggctacaatcagtagtgttaaggtacttcatgcagacctcacgagttaatggattaacgagtggggtcttcgcatttctggtac
ctgcgtcttgcctgaaccagaaaggctcaaacacccgccgcgataataccacattccctgttgctgactattacccccatttgcctgtttttgttacaaaaac
gccgggagcgggggaaaacttactttttcccgtttccgggggggacaactgagcattgcgaacgcgtcagctacgatgttccggtgattgctgatcagcccct
gttgtattcataaactaattaacattcattctgtctggtgcataaatctgcccgtgaacatctgcagacaataccggccatgaaaagataaataataccagtg
gaatatatttcctcatgttattctccatatcagataaatggcacctgtatttcagcccccctgaatatcgcagcacacattgaataagaccttattttgtgaa
attaatcacaaaatataaaatcgcgacttaatacggaaaaaactctgtgctactcagaactggccggtcatactgcccgaccggatattaaaaaaagcaaatc
tcataaactcctttcagccttctgactcctggagtaatgaaggctgcttttttcgttacacccttccccggcagcgccggtggcagtgttcagcccttttccg
ggcaaactgttgccagtctgtccggggagctatgcaggcagcccgtcgccatgcttctgccgcttccatccagcgtccactgacctccagaataacggcccgc
ccgccggcacgctggtaacgccagacctgaccgttccgtgaactgtttctgaatccattcatttttctgctcctcccttttagtcgtcagtattattgtggta
aataattcagtggactggcttcctttcgggtacggaagttatttccttttaagccttattgtttcagcctccccggtttccggggaggctttttttatttccc
atcaagcacggcctgaagttctgcctttgtccggttctggggcgaattttctccaaacgccctgccgcatacgcttcctcctcctgagaaatatgctccgtca
catacccttccgtgtcataccggcaggcaccggctttcatggcacacagatagctttctgaacgggtgatggccttcagcgcccggcgcagttttttatgcga
gagcgggatattccgctgcgccacgtcttccagtaacacgtcccggataccgcaggccagaagccggggcgtgttaccgtcaaacaatcccggccaccaaggt
ttcagagtgttcaccgcctcatccagcgagggcagcgtcagataaatggacagcgcctgtctggcctgtgctttcttcgccgccagctctgcttcccgggcag
ccttctccgccagtttctgctttttcaccttccattttggtggcgtggtgacattgatgatggttttccggctccgggtcggtgtttccccttccgttttccg
tttcagtgtcagtaccggtcgtttctgctctgtcatgattgccctgacctttgtgaaaatatgtcctccaggtcgttgaaactactcactatgatattaagct
gacctcaacgacctctgacatattaataccgacaattaaagatgttttttaaaccattgcgtctgtactgccgccacacgttcaaaaaacgcgcctttataaa
tatcataatggcatgcgtcagcttcctcgtataattccttagtaccagaagcaacagcatcatataaggctcttccttgttctggtggattaacactatcctg
gccggcaataacaaccaaaactgggcactgtactttggctgcgctctctgccggtttatattgcaacgtctccataaccgtaaggaaaggtatttttatatcc
atttcaggatactgaccttttaccttctcgaagaatacctttgattcattatcactcagtacccgggtaacccccacaaacatctctttcccggtatttttct
tcttttctgccattttattcagcgtggacagaaaagaagctttctccagttcattcatttctccggtaaccagaacctcaccgtcagcaaaagccagctggct
gacaatacttttcactcttttatcctgcgcagctgcattgaatacatgacacccacccagcgatgttccccataatcctatacgctgattatcaatacatacc
tgcttttctgcccaattgataactgaaataatatcttcggtctgcattgccggaaccagacgacctcgttccccttcactctcaccgaatccacgataatcaa
aagtaatcgttgcaaacccggcttctgtaaacgcattcgcaaaggaaggcagcagaacattccggataccgcaaaatccatggcataagagaatcaaaggatg
ttttatatttccttccgggacacgaaatgtcagggcaataccttcagggagtttgtgatccgtaattttcataaatatcctgtgttatgttgtttattaataa
gcagaatcagcgccagcatcataccagATTTgccatcgtccacatatccacgggctggcaagggagcgcagcgaccgcgcagggcgaagcccggagagcaagc
ccgtagggcgccgcagccgccgtaggcggtcacgactttgcgaagcaaagtctagtgagtacgatataagttgttggaagatcttccctaaatttaaatataa
acaacgaattatctccttaacgtacgttttcgttccattggccctcaaacccctaattaggatcaataaaacagcgacggaaatgattcccttcctaacgcaa
attccctgataatcgccactggactttctgcttgcgcggtaaggcaggataagtcgcattactgatggcttcgctatcattgattaatttcacttgcgacttt
ggctgctttttgtatggtgaaggatgcgccacaggatactggcgcgcatacacagcacatctctttgcaggaaaaaaacgctatgaaaaatgttggttttatc
ggctggcgcggagtggtcggctctgttctcatgcaacgcatggtagaggagcgcgatttcgacgctattcgccctgttttcttttctacctcccagtttggac
aggcggcgcccaccttcggcgacacctccggcacgctacaggacgcttttgacctggatgcgctaaaagcgctcgatatcatcgtgacctgccagggcggcga
ttataccaacgaaatttatccaaagctgcgcgaaagcggatggcagggttactggattgatgcggcttctacgctgcgcatgaaagatgatgccattattatt
ctcgacccggtcaaccaggacgtgattaccgacggcctgaacaatggcgtgaagacctttgtgggcggtaactgtaccgttagcctgatgttgatgtcgctgg
gcggtctctttgcccataatctcgttgactgggtatccgtcgcgacctatcaggccgcctccggcggcggcgcgcgccatatgcgcgagctgttaacccagat
gggtcagttgtatggccatgtcgccgatgaactggcgacgccgtcttccgcaattcttgatattgaacgcaaagttacggcattgacccgcagcggcgagctg
ccggttgataactttggcgtaccgctggcgggaagcctgatcccctggatcgacaaacagctcgataacggccagagccgcgaagagtggaaaggccaggcgg
aaaccaacaagattctcaatactgcctctgtgattccggttgatggtttgtgtgtgcgcgtcggcgcgctgcgctgtcacagccaggcgttcaccatcaagct
gaaaaaagaggtatccattccgacggtggaagaactgctggcggcacataatccgtgggcgaaagtggtgccgaacgatcgtgatatcactatgcgcgaatta
accccggcggcggtgaccggcacgttgactacgccggttggtcgtctgcgtaagctgaacatggggccagagttcttgtcggcgtttaccgtaggcgaccagt
tgttatggggcgccgccgagccgctgcgtcgaatgctgcgccagttggcctagtactcaagcAAATcaaagcggtgtcggcgcgttgttgtagccgcgccgac
accgcttttttaaatatcataaagagagtaagagaaactaatttttcataactctctatttataaagaaaaatcagcaaaaacttgtttttgcgtggggtgtg
gtgcttttggtggtgagaaccaccaacctgttgagcctttttgtggagtgggttaaattatttacggataaagtcaccagaggtggaaaaatgaaaaaatgga
tgttagccatctgcctgatgtttataaatgagatctgccaggccactgattgctttgatcttgcaggccgggattacaaaatagatcctgatttactgagagc
aatatcatggcaagaatcccgttaccgggttaatgccatcggtattaatccggtaacgggatatggcagcggactgatgcaggtagattcccagcattttaac
gaactggcccgctacggaattaagccagaacatctgacaacagatccctgcatgaacatttataccggtgcttattatctggcaatagcatttaaaaaatggg
gcgtcacctgggaggccgttggtgcatacaatgccggattcaggaaaaccgaacgccagaaccagagacgtcttgcctacgcatcagatgtttaccggattta
tactgggataaaaagcagtaaaggtatccggcttccggccacgaagaaatcacttccggaaattaacagcgtgcagaacaattaatcttacagacattgtcag
gataccggttatgccgcaaaagcggcatggaaggccacacacaaaaatatcatatgagtacaatactgactggccgaagccccgcgaagcgggaacccatctt
atatatacccggtaactgtgcagcctcctgccagaccatttttcgcgggcacaatatttacagacaaccggaaatccggttgatgaaattgaggtatatcaga
cgacacagcaatgccgaaaaatgataatataagtaaagccccgaaaattttttcggggctttacttatattatcatgacttccactttctgaactgttcggca
atcacccataacgccttgttgattcttatatcaccatcaatacctgtaatggctcttgtcctggtatttttcccggaggcacttcgccccgataatccacctt
ttatcatattctcctgaacccgctgccaggttgtccagagatcgttctgtttgtcttcccgacgacggggagtaattatctgttcaggtgtcactggcgtttt
attttcatcttcatacctgaccatcagtgcagctctgccaaataaatgttgctcgtcactgttaagatgaatttctttcatcgcctccatattatcagtgacc
ttatcaaagacaccgagcacttcatacgctccctcgataacctgcccgacaatatcacctttatgtggaacgcggatttcgccaaaattatttccgcacacca
ggccatttgtgcagacaaaacgaaaaattccggggatcatctgataactggatgaaccatcatgtgaattaagcaggataatttcaggaacttcctgtccgtt
aatatgcccttccctgcgaagacgtaacatatgtttactgtattcgcgacgtcccaaatcacgaatccgactctgacaggcaaagaatggctggaaaccttca
tcacgtaacttattgatgatattgattgttggaatatacgtataacgttcactccgggactcatgtttatcaccggaaaataccgaaggcacgaactgcatta
attcatcatccgttaaaggacgttcacggcggatggaattataccgaccaaaacgggaagctaatcgcataacagctactcctttatgaaagaataaatttaa
taaaaaatttttccgcttaatatcatccccccgatctccattaacaagattgcaacaatcaggagggatattcatcacatccggtcagcagatcgcccggata
tcgaggccggcacgcttaatctgacgcaggaactgtttgaagtgatcggcttccgcaatgttgatatggcggaactgcttatccacaccctgactgagcgcgt
agctataactgaaggccatcttttccgggtggcttttgcgatattttttcagctcagtcaggctgctgaattccaggacgtcccactcttccccggtgttttt
tttcttcccggcaacaagaagcgtgtacgacgcggtgttaacgtggatatttcgggtactcatcctgctttctcctctctctggttttcagcccccatcatct
gttgagtttcgggagccgcggttttgacgttttgcggggacctgccttttgttccctccggtattcaccatcacctgtttgagaaccttcgccgcatggggca
tcactgccacggccggaaacgactgtccgggggcagaccgcaaaagtttttgcctgcttctgggcaaaaagttttgcgggatgtgcatttcacccctggactg
gcggagacggttgtggctgtctatgcccccggcgacaggggcttaaactggtgaatggcaccggacacaacaacgggcagggacccgccgcaacgacgtgaag
cgcggtttgcgaaacggcgttgcgccttacatcccggcgccttttgccgggatgccgggagcgcaggtcacggcgcgacagaggcggcgcgaccggaacggtc
gctgcaagccaggggttttcacgcatcggaccgtagcgacagcccgaaaccgcgccagcgggttcggcggagactggcaggtgcgccagcagctgcctgtcgg
agtcgggcctgacgaccggatgccgccaggcagccgggagccgaaggtacgcccggcagacaggacaacactgcagtagtcagaatcagagcaggaagaaaaa
gagaataatcacagggaaggtgatgtgatttctgacaggccataaccagagaaggcgggggaaaagaacgggcgccgcaggcgcgcgttcatccccgttcctg
aagggaaacgaccttcctgaagtcaggttgttcatgcccgtacaacatcagcaaggagaaaggggctaccggcgaaccagcagcccctttataaaggcgcttc
agtagtcagaccagcatcagtcctgaaaaggcgggcctgcgcccgcctccaggttgctacttaccggattcgtaagccatgaaagccgccacctccctgtgtc
cgtctctgtaacgaatctcgcacagcgattttcgtgtcagataagtgaatatcaacagtgtgagacacacgatcaacacacaccagacaagggaacttcgtgg
tagtttcatggccttcttctccttgcgcaaagcgcggtaagaggctatcctgatgtggactagacatagggatgcctcgtggtggttaatgaaaattaactta
ctacggggctatcttctttctgccacacaacacggcaacaaaccaccttcacgtcatgaggcagaaagcctcaagcgccgggcacatcatagcccatatacct
gcacgctgaccacactcactttccctgaaaataatccgctcattcagaccgttcacgggaaatccgtgtgattgttgccgcatcacgctgcctcccggagttt
gttcggcaccatccaccgctccatctcacgaacagcatgaccgaccacccttgccgccataccagcctctgcgatgacctgccagaatggcatatcctgccac
tgccagcgttccattccggccagtgacggaaaacgctcgccccactgccagaacagggtttccaggtcatgctctgccacccggtcactccagcgtgcatacc
agcttccgactgtttccggcgaatggaatgccagttcgatttcagcctgagcgaccagcgcctggtacgcacgggctttctgctctgcctccctcgcctgctg
gcggctgtacttctgcatgaccagcgcggatttatggcgactgcgttccctgacacgaaaccgcaccgacgggaacaagcgttctgctgcatcgcctggaagg
ggaaggaggcaaacctcgccctgtgtttcaatcagcacatcgatggcatacagccactgaagacggttgccgtgcagttcttctgcggtcagggacggcatga
aaaatcgcaggtcagacagcgatatcctgcggcttccgttcagacagcggaaaaacgcccccgcatacggatattcagccagcgtgttaccctgatggcggcg
tttttcggtctccgccaccgcccgccatgcggcctgttgctctgtgctgagaggaaccagtgcctgtgaacgtgcgctcatgcctgacttccttccgccatca
ggatatggatgatgtcactgacgctgtacccctgcgcgtccagtgacgccgccagtgccagtgaatggctgacagcttccggtgcaaaccgccgctgtacctg
aaccaccgcaaacggctcaccaccggcattcaccagaaccagcacccagacctgagagacatcgcccggtgaacataacgccaggtgaaaacgttcgtgggca
ggcgtgaaacggacctggacggggaaaaagccgccaaactcgctgccgtactcgccgttcatcgtccagttatccggcgcatccagctcccgcatcacggcgt
gagtcagctcacggcgttcatcacttccggcagcccggatatcttcatattcctgtgtgttcatggtctgtaatacattcagggtcagttcagttttcatcac
ggtatctcccttgctgccgccccgtctccggcggggcggtgtggttgttcatcaggcggcatcagccaggttatcggtgcgttcagtggcatccgtctgtggg
cgtggtgcacacatccagcctggaacccagcggttgtctttcatatggtgctctgcatgttcagccgcatcacctttcttcatcttctccgcgtcccgtgcgg
caccggacagccctgccttattcagggctgcgataatctgcggcggacgttcgcgcgtcgggcacctctcttacttaagaatcccgaaatccctgatgacatg
gttcatccagtacccggcaccgtcctcaaaattccaggtacgccagaccattgcaccgttattacggacaaccaggcgaaactgttcgccgtggtcatcctcg
ataaagacgttccggtactgtgcggcaacagcttcggcatgtttgcgggtaaaaggaccgtcaggcagcccctgaagtattctgaaatattcgctcatcctca
tctctccggtgatgccccgcacagaacggggggggggggcggttatcacagtcagcccgatcagaacgggatatcgtctgaaaacccgtaatcgtcaccctcc
ggcggttgcggctgaggctccggctgcgcggccttacgtccacggcctttcgtttttgcgccaccttttttcgtaccggcctccgcctgtggttccggctgag
gctgaccgctgaactgttgcccctcttccggctgagtctgagcacctgcggcacgtcccagcatctgcatggtgcccgtggtcttaacaagaatttcggtgac
gtaacgggtaataccgttatcctcccagctacgggtgcgaagctgaccttcgatgtagacctgcgcgcccttgcgcagatattcgccagccacttccgcgagc
ttgccgaacagcaccacgcgatgccattctgtctgctcccgcatctcccctgtctgtttgtcacgccaggtttccgacgtggcgacctgcaggtttgccactg
cgcccccgttggggatgtaacggacttccggatcttttcccaggcgtcccacgagaatgaccttgttaatgccacgaactgccatgatgtgcctccttctgtt
gatgttttcagcccccatcatcttttgagtttcgggagccgcggttttgacgttttgcggggacctgccttttgttccctccggtattcaccatcaccttttt
gagaaccttcgccgcaaggggcatcactgccacggccggaaacgactgtccgggggcagaccgcaaaagtttttgcctgcttctgggcaaaaagttttgcggg
atgtacatttcacccctggactggcggagacggttgtggctgtatcagcccccggcgacaggggcttaaacaggtgaatggcaccggacagaacaacgggcag
ggacccgccgcaacgacgtgaagcgcggtttgcgaaacggcgttgccccttgcatcaacgccagcccggctaagatggaggcacggcacagcccgcacacgac
tcgacgtccacataactgaacccccacaaggccatgctccgcatggccgggtaaagcggagccggcggcgacttcctttgtgctctgtccggggaccggcagg
aaaaaggggggattttccccgccatcgtcatcatttccggatttttgccagggcatccgccatgctgcggatttcatcccgtgcctccaagaaaactcccgca
gaatatagcggtcaaacaagcggggggggttcacccggtatacgcagcacatcacagaccaagccactggccaagatatgttccacacactcactggcaggca
ttaatggaatcgtagtctcacgctgataatcagatcgacaatacaagtgagaccgtggtcccagactgattagcagaccaacaatacgagtgggaccgcggtc
ccagactgattagcagactaacaatatgagtgggaccgtggtcccagaccgataatcagaccaacaatatgagtgggaccgtggtcccagactgattagcaga
ccgacaatatgagtgggaccgtggtcccagactgattagcagaccgacaatatgagtgggaccgtggtcccagactgattagcagaccaacaatacgagtggg
accgcggtcccagactgattagcagactaacaatatgagtgggcccgtggtcccagaccgataatcagaccgacaatatgagtgggaccgtggtcccagactg
attatcagaccgacaatatgagtgggaccgtggtcccagactgattatcagaccgacaatacaactgggaccgtggtcccagatataatattcaggccagtta
gttatgttttctagcctgtgctaaaggaaattaagtaaagacagataaatgtagactaaaacgtgccgcatcaaagtgccgctttttcaagttccttaagaat
ggcttcaattctctctatacactcagttggaatacgagatctatctaggtttaacaccattttatcgcccttgtacaacactgtcgctccaggagcaaactga
tgtcgtgagcttaagctagttcttgaggcagatgacgttttaagcacagacgttaaaagagtgatgacttcttcagcttcaaatatcactcctgcttttttaa
ctgctcgtgaaggttagatgcctgctgcttaagtaattcttctttatctgtaaaagctctttgaagtgcatcacctgaccgggcagatagttcaccgggatga
gaaaagagcaacaactgatttaggcagtttagctgtgttgatacagcgggtaataatcttacgtgaaatattttctgcatcagccagcgcagaaatatttcca
gcaaattcattctgcaatcggcttgcataacgctgaccacgttcataagcacttgttggcctataatcgttacccaatctggacaatgcagccatctgctcat
catccagctcaccaaccagaactcgataatcactttctgtaagtgcagcagctttacgacggcggctcccatcggcaatttctatgaccccagacactcttcg
accgaatgctggtgtctgctgacctgtcagtagaaaagaagggataagatcatccagggcgtcttcggtaagcagttcctggtcacgttcattacctgaccat
acccgagaggtcttctcaacactatctccccggagcacctcaagggtaaatttcacgtccctaccacatacaggcaaagtaatagcattgccacgagccatag
ctcctacgcgcgcaattaacgaatccaccatcggtgcagctggtgccgatgacggagtatcttcaagcggttgagtcttaatcgtatgttttggaatgacagg
cgcacgcttcattatctaatctcccagcgtggtttaatcaaacgatcgaaaatttcattgcagacaggttcccaaatagaaagggcatttctccaggcacctg
ttgaagagcgttgatcaatagcctgttcaaaaacagttctcatccggatctgacctttaccaacttcatccgtttcacgcacaacattttttaggaccatgct
tccccaggcgtcccgaatttgctcctccatccacggggactgagaaccattactattactgtatttggtaagcaaaatacgtacatcaggctcgaatccttta
agatctacgtttttgagcagatcacgaagcatatcgaaaaactgcagagcggaagtgtagtcgaacaactcagcaggcgtggggacaatcaacacatcagcag
cacatacaacattaatcgtgccgatacctaggttaggcgcgctgtcaatgacaatgacatcatagtcatgagcgacggtttcaatggccagtcggagcatcag
gtgtggatcggtgggcaatttaccttcatcaaatttgcccattagctcagtttcaatgcggtgcagagccaaacaggaaggaataatgtcaagcccaggccag
caagtaggctttattgcataagtgacatcgtccttttccccaagatagaagggaaggagagtatcctctgcatgaatatgaagatctggcacccagccgtgat
acatcgatgctgttccctgggggtcgttaccttccacgagcagaacacgtaatcccttcagagccagatcctgagcaagatgaacagaaacagaggttttgta
aacgccccctttatgagcagcaactccaatcaccggcggaaatacgtcttcagcacgtcgtagtctcgtaccaaacacgtcacgcatatgattaatttgttca
attgtataaccaacacgttgctcaacccgtcctcgtatttccatatccgggtgcggtagccggcctgctttctcggcatccctgatagcctgagaagatactc
caactaaatccgctgcttcacctattctccagcgtcgggtgattttcctcgcttccgggctgtcatcattaaactgtgcaatggcgatagccttcgtcatttc
atgaccagcgtttatgcactggttaagtgtttccatgagtctcattctgaacatcctttaatcattgctttgcatattttcatttaatcttgcattttattgc
aaagcaacaacaaaattgcaaagtagtcaaaaaaccgcaaagttgtttaaaatgagagcaacacatcaaaaaaggaaacaggaagagcacatacctcagtcaa
taattatcactagtactcgtagccgtcgtgtaaccgagcgtagcgagctaactggcgaggacgcgcgctaactgttcacactcagtatccctagcaccaaaat
aactatctgatggtcaaggcggtaatgactccaacttattgatagtgttttatgttcagataatgcccgatgactttgtcatgcagctccaccgattttgaaa
acgacagcgacttccgtcccagccgtgccaggtgctgcctcagattcaggttatgccgctcaattcgctgcgtatatcgcttgctgattacgtgcagctttcc
cttcaggcgggattcatacagcggccagccatccgtcatccatatcaccacgtcaaagggtgacagcaggctcataagacgccccagcgtcgccatagtgcgt
aacaaccgtcttccggagactgtcatacgcgtaaaacagccagctcaccgaatacgtgcgcgctggcgcgatttagccccgacatagccccactgttcgtcca
tttccgcgcagacgatgacgtcactgcccggctgtatgcgcgaggttaccgactgcggcctgagttttttaagtgacgtaaaatcgtgttgaggccaacgccc
ataatgcgggcagttacccggcatccaacgccattcatggccatatcaatgattttctggtgcgtaccgggttgagaagcggtgtaagtgaactgcagttgcc
atgttttacggcagtgagagcagagatagcgctgatgtccggcggtgcttttgccgttacgcaccaccccgtcagtagctgaacaggagggacagctgataga
aacagaagccactggagcacctcaaaaacaccatcatacactaaatcagtaagttggcagcatcacctttattcattataaaatgccccgtaatatttatctc
acaatgattatgattagtggatacgcccctacatttccagacagctgttatcacttaatctattactggcccgcagccgtagatattctagccacggtggtaa
cgagtatccggaagagattccacttacactctttcaatcctattgcaaaagctgatttactcaacaggattaaaagagggagtttgatacaatttatttcact
ttttgttagaatactaattcttttcttgtatttttttgaggaaatgagcacggaacacgacataatctgtagtcagtaaagagtctatcctccctgactacag
attatgtcgtgtattatatgatatcaacttggctgtcagcggccatgttgatatcagcgtacctttgttgtaaaggaataccggtcagaacagcacagagtag
ttcagaccaaaggttcggcccctgcctttgtagtcgtacagtgacgctgggccgtaacccgggctgtagtacagtggtgcacgctgcccccagacagtggtgt
agtcacggtcgaagaggttttcaatgctgaagctgagtgtacccaccggaagctgataactgccgagcagatccacggtggtatagccatcgaccttgtagcc
ctgcgcgtcgctcacgtcaaaggaggtggtgctctgcacgcgcagactccacgggtccggtgcccagccaatgtaggctgtcgcttttgatgggcttgctgtc
ttcacatcgtatttctgccaggtaccgttcactttcgactcagttttcagcacgttgaagttcactccggtactccagtcagtatcaggaatcaggtagtcca
ccgcaccttccacgccgtaaatacggcgtttgtcgtccaccacgctgatggtcagatctttattcgccaccacgctcttatcagaaatcgaatagtaggccgc
gatttgggtacgcagattattgccagtaaagcgccagcccagctcataagaatcgactttcacgccttccagcttgctgtcgctgacgttcacactctttgtt
agaggaagatggccgttcactgcagcaccatagatgccgcgaccatagtatttacccgggtccggcagctccacgccctgggagaagttgagccatgcctgct
ggcgttcggtgatgtgcatcagcagaccggcgttgaacaggaagttgtcgtaatcgactgagccgccaggaatggcgtcggcggatgtagccttcccggcggc
aatctgccgttgctgcgcgtagccgatgaaatcatcaatcttgttttcagtgtactgatagcgtacgccaccgttgagggtaaagagattattgatgtcatag
cctgattgcaggaagaccgccaggttggtgatgtcatacgacggatagcgcccggtggtgtaaatcttcttgttgttcagccctccggaagcgcttgcctgag
ccaggtcgaagaacatctggttggaggtaaagcgctcatgatcagcatccagcccccaggtgatttgccagccgtccatcggtttgctgttcagagtcagttt
catgccgtactggtcggtgtcctgctgtgacgaagagaaagccgtcacctgtttgttcgcatttaccgtcgggaacgggtagaatcgcaacgactcatcgcgg
tagtaaacctgaccgaccagctcctgtcccagaaaagcgctgtcagagtactgcaggctgatcaaatgccgctcagtgccgggaatacggtcggaattcagcc
cgttactgacgaatggcgtgctggtccctctgatggcagagaagcctttcccgagattaagcccgtaatcgtcgtcgccctggcttttatagtactgtgtgat
caactgaagctgccgggattcatcgatgttcagcgtaccagttcccatgatgtccagccgatcggagtactgcaggccggtctgggtgttatcaagcaaggtg
gcatcgccgttaccgtcaaaccagccgccaaatttctgatatgccacggaaagacgtccggagatatgctcatttccgccggagacagctccggcaatgcgtt
catcgtgatctttactgctgctaaagccacttttggtgccagcctcaaactccatcatggtttccggctggccttttttggtcacgatgttgatcaggccacc
ggtactgccgccgccgtacagggacgtcgcaccggagatcacttcaatatggtcgatattaaaaggatctatagagtccagttgtcggctgtcggtacgtgaa
gagttgagacgcacgccgtcaaccagcacgaccagcgggcggccacgcacattcataccgtagttggtgcggctccggctgctgacgtcaaggccagggatca
gctgagccagtgcgtctttaagctctttgccgccctgaatctgctgttccagttcggcgttttcgataacccaggtggtttgcgccatctccgctacggtgcg
attgctgcggttggcagacaccacgaacgtttcatcatcggtttgttgagcgactgctggcgccatcatggtaagaagcagtgggttgagagcccaaagcgta
tactttttgcttatcatcattattcctccatacgggcggaaaaaatcagcccaaagatgctgtgtagcgagttaaagaagcagcctccggctgcgttttccct
aggtgccgctgcgccactgaatcaggggggggcatactgagatcgaataaatcacgtcccattacgcgattaagaatacgtgcagatctccatgccatcaggc
tgagctggggttcggcgatgccatgggtttgcatactggcgttgaccacgaagatgttgttctctttcgggccactccattccagagtgaagtcatcacgcac
tttaaaggtgttcttatcgtgcatggtgatcaggggcatcagtgagggaagtatttgtggcaacgcagaacggtaacctgtggcgaaaatcaccacatcactt
tccaggctctccctgccctgatccagatgatgctccatcagcaacttccagccaggaccactactttccagagttgttaccgagcggctgggtagcagacgga
tatttcttggttttctcagaacttcaaaacggtggtacaactcacgataaatggtcagtaaagaatcggcagtgatgccatccgatgtcattttctgctcatc
cagtaactgatggcgaatatcttcctccagtccggagaagcctgaaatatattcaggtgtaaaatactcatcagcaaaagcagcctcatccagtgcgttaaaa
ttattacgccgggagacccagtttatttccgccgcttctccccattccccgcgtaatgcattaaggaacaggtctgcaccactctgtcctccaccaaccacgg
ttatccgttttccactaaggtccggccgacgaagattcatttcactggcatggaaacaggattgtgtcatatgcttcacacagggtggtaaataaggttgttt
tcctgtaccaaggcagatattgcgggcaaaatattctccctggctggtttgcaccagaaacaatcgacgttttttatcgaaatcaatgttttcaacggtatga
ctgaaatacaggttattcatatcttcagcagcccagcggaggtagtcagaaaactcttcacgggatactgtacgtagtctgcttgtaaggaagcgatagaact
ttttgtgcttcaccagatagttaacaaaactgtagggatttgtaggtgcaacagcactgaccagatctttcagaaagacggtctgcatatgacaatccggtac
cagcatacccggatgccaggaaaaatgaggatgttcatcaaagaagagacagtccagttcttcgatctgatgtgacaacgcagcaatgctgagattaaatggc
cctgtccctacaccaataaaatcgacactttttttcatgattcatattcctgagttaccagccacagcggattttgcagatcctcaaggtaattcggcagcat
gcggctgccgccatccagatcaggccaggtcagttttaccgggttcagcaccacgcgaatgatttgtggcctgaaaagtgagaaaagcgcaaaacgctctgac
atttgtggatgttttttcatgtaatcactcaacactgctgccagcagttgataaaatcgcctttcaggtacgccaagacgaaccatcagtggcgaaataaaac
gcagtactgtcacgaagtgacccgtctgcaaatcatggattaagtagtccgcactcaggcgggatgtaacatcacgaacctcctgaggcaaagagtccatttc
ggggaactcttctttcaccagtcgcatatcgccctggaagtctttcagcagaacacgctgtggaaccccctctttcatggcgagagttatattttgtccatgt
gcaataagcgcgacaccgtagcggcaaagcaggtgatacagaggaaccacgaccacccggaacagttgcgtaagccaggtttcagcgtccagcccggagcggt
ctatatatgcgcctgccagcggctgattgttttcgtcgcactccatcagtgttgccatcagaaccggactttcatccggtttcagccagcggcacggattctc
ccgccagataacaccaagcatttcctggtagcgatagggagcccgggcaagcgcggcatagccttcatgggagacatagcctgcagccggttcaccaagtatc
actgcgccgctttgcactagggtggcgtcggtcgcaaaaacctgttgtagccagcgtgaagccagtggtccggcagcgatgtatctgccagggatcccccggt
agcatgaggtgttgtagatggtcagcggcagcttgatatccagcccgccccggcgactggcgttggtcagggtacgcagcgactgctgggcgagccactggtc
gccaaactcgccgagagacaccatcctgccttcggcaaaatcagcgatgaagtcggtagcgattttttgctgccactgccacggatgtaccggcagcggcagc
cagttatgatccagtccgttttcctgccagacctgactgaagcgggcaaactcctgcggatccattgcggccgtcaacaactgatgaatatccatctcgttat
cacagcgccagatcatatgttcacgttttaccgccagccagtgcagtctgaaggtgttggcatactctggcgcatatcgttccagcgcctctttaccccagcc
acggcgacctttattaaaaacgaatttaggatgaccgctcagcaggcattgcaggcggtcggcattaagattaatcaggtcactggcgctcagcccgcgacgg
gctttcagtagttgcaggtcgcccagcagcgtggcatacaaatcctgcatatgctcagcaacggttgcatcgctcattgacagtacctgctttagctgcatca
gcagcgtctgagccagtactggctcgtccgcgcagcgcagagtttgagcatcaatccagagccagccccagataccacgttcagcgatgaagcgccattgtgc
tcccggcaggttaatgcagtagcggtcatcgccctgagattccgcgtggaaaacctgctcatactccagctcagacaacatttttgccaccaggcggcggttg
accaaatcccagtctttgtgattcattacagaccgacctccgtgaagaagtgatgacgatccgccatcaccatgcgcgagcgtttatgtgggaagtcgaactc
tttaattgtccggtatcccgcaggctcaagatggcggaacaggcgctggttatcggtgcgtggctccagtactgtgcgctgcgtacgtggctcattcagcagc
aggtaatgtgtcaccccgcgcagccagctttgcacatagtgggctccgcgccattgctgttcaccaaccagcagatgcaggccacggtcaaagggctgccacg
aatagtggcgaccaatgcggtcttccgccgcccagtagatttcgaaataactgaacggccgatcgtcgaaacaaccaatcagcgggaatgcatgtttactggt
gagctggcgttccagataggcgatctgtacctccagtgagccactttgctcccagaaatactcaacgcgcggatcgttcatccagcgagtgaaacgttctgca
tcagaaacgggatcggcaatgcggaaactcagcatcctgcgaatgcgtggatcgtaacgacgataaacttcaccctgcggacggggggggcgctgcgggaaat
aaatctcacgctctgcatcaaaaaccattttaccggaggcccggtttgccggagaactcagccataacggtaactgccagaaagcggaacgatggataatgtc
gctttgtacctgtccgaaaagcgtcagcgcctgtggctcctcacaccattcggaccagggaagaaccactgctgaaagttgtggtgcggcgataaatatctga
tccagcgcgtcgcacagccaccctgttggcagctccccgggccagtgcaacaccgcgctattgtccagcccccagccaagattcagaggcttgtcgagttttt
cacagcgcagtccatatccgctgtgaacaatgtttgccccagacattcagacctcctgagcctgcagcgggttagcgaaatcaaaatagatcaccgaagggtc
aacgatggtgttctcgttatgatcgttcagataacagaaaaagttaccttttacgttccagtacggactttccagaacatagttgagacaggttttatgagtc
acctggtcacgcacttcagccagcaatgttcgtacacgagccatcagattcgcttcgctgtccagcccggcagcacccagcgcagcagttacggcaaaagtgg
agttaaccagcaggtaataagggaaatagcgcagcaactgctcacgggtgaaaatattttccgcctgcgcctcgtcaatggtatcgagccactctgtcgcgtg
aggcataaacgcgctgccctggcagtcacggtaaataaatccgaccggcagatccccaagcatctgcacaagaatattttgctgatgcgccagcagcaccagg
ccgtaatccgcttcagccgtaaacagaggcttcagcacctgctgacagtaagcatcgacccatgcgtgggcggcctgttgcacagtgataccgaggcgatcgc
tcaggcgttttaccgccgaaaccagcagcgaatcaccgccatccggtgcggcctgagtcagggaaaccagaacgttggtctggctttgcggctgctccagcag
caggttttcacgcagggaaaacaaactttcctgcatgatgttgccgttaagatcgcgcagcccggcccagccgtcctcctgcattacccggaaagtagggaag
cgggcctgtagcatctgccagccgtcggtttgcgccagacgtgccaggcgcattcctcgctccacctctttcacagacagagtacggacggagttggtcagcc
gaacgctcagggagaacttgatcatatcgcggctggtagcacagtagagggagcgggaagaggtggtcggcagccacgacgtgccggcctcgcctaagtctct
gataagtcctttagcaaaaagtgcctggcaccacacttgctgcaaaagatattctccctgccacgggtgcagcgggaacagccattgattgtcacttaattcg
ttgagtaactggggcgcattttctgcggcaaatcgcgtcaaccgctgttgaaggttaagatgcagactttcaccagcgatttgcgttttatccaccgaaaacc
accgcagcgggaagtgaggtgccatgtcaggcaggtatcgttcagcttcctggcggttaaacggttcatgagacttaggtgcagggtggaaagcgtgtcctgt
cagcagtgcctgctcagcctcgccaaaattcaacgctttttcacgcaggatggcccagtcatggcgggcatcaattgtctgttgcgtatgggcgtagctttcc
agcacgcgttggtggaaagtttcgcagaggggaactggcagaaaaagttgatgccggagcttatcaataataagccgggagagagtggtaaagtcgacaggat
aactgcctgatgccgtgaccagatgagcaggaaatgcaaagcggtgatgctgggttggggagaaataagccactctgaaatggagcgacttctgttcgtccag
cgggataatcaattcgtctggcgggtattccgccagttgccagtctttggtttcacgaatcaaggcattcaggaagcactgcgcagccacatctgtggcggat
ttttcagagggcaggatcataatcaaacagctcctacacagtaaaataataacaattatgtcaataatgattctcattatcaatgagaaatgttattgtcaga
aaaattaattttacaaataaacacaattaatacatggtgttgaaacattacaatgtcatctaagatcgaagatacgccccaaaaaaccctgtcctgctggcca
ctggcgttcagtgccggtcttctcggtatcggacagaacggtctgctggttgtactccctgttctggtcatacagacaaatctgagtctgtctgtatgggctg
ccctgctgatgctgggctcaatgctgtttctgccatcttccccatggggggaaagcaaatttcccttactggcagtaagactgtggtgctgtgggctctggga
ggatatggcgtaagctttaccctgcttgggctgggaagtgtgctgatggctaccggtgccgtaacaacagcggtggggttgggaatattaatcatcgcccgga
tcgtctacggtctgaccgtgtcagcaatggtgccagcctgtcaggtctgggcattgcagagagcgggagaagggaatcgcatggccgctctggcaaccatcag
ctccggcctgagctgcggcaggctattcgggccgctgtgcgcggcggcaatgttggtcattcaccctctggcgccagtgtggatgctgatggcagctccggtg
tgggctgtggtgatgcttctgcggttgcccggcacaccaccacagcccacaccggagcgcaagagcgtcagcctgaagcgggattgcctgccttatctgcttt
gcgcaatgttactggctgcggcaatgagcatgatgcagcttggactttcgccagcccttactcgccagttcgatactgataccaccactattagccaacaggt
ggcgtggttgttggggctgtccgcaatagctgcgcttatcgcgcagttcgtggtactccgtccacagcgcctgactccagtggctctgctcctgagtgccggg
gtgttgatgagtagtggtctggctatcatgctcactgaacagctatggttgttttacctgggctgtgcagtgctgtcatttggggccgctctggcaacacccg
cttatcaacttttactgaatgataagctggccgacggcgcaggcgcgggctggcttgcggccagtcacacacttggctatgggctgtgcgcattgttggtgcc
actggcgtcgaaaacaggtgtctcaatcgcgctgattgtgacggcattatttgccgctgtattatttaccatagtgtctgcatgtatttggcattaccgtaca
atcaagtaaggctatgatgttgtactcacagccggcataacgtcctgtgcaatttcgctgtcgaaaccgcctgtgctgaccgcccagtgcactcacctgaagc
acatcgcagcatgccaccaggatgccagatcgtcttctgaaatgggtggatacattggcaacgttaaccgcaacaatttttcagagagaattttggggggtga
gggccagtgatgtaaaaaatacgccaagaggttaattcattgtttatactgatatgtcaggtaatgcgtttgcagtcattacagtcgtcatggagactgtaat
gactgcaaacgcataccatggagcgaaattctatcctaagcgtcacattagttgataaacctgtaacgttaatgaattctcatgcgaaaagtatggttatgat
aaaaaagcctaaagctgtcatgatgagggagccaatgacgtgaacaagtacagacgtcaaagcccatatataatttcccgcctggagtaatgcgaatacttca
acagaaaatgtagagaaagtactcagacctccacaaagtcccgttgttatcattaatttccaaaaaggatcaaggtgtggttgtcgtaaaaagtacgccaacg
cagtaccaataattaacccggcaaggaggtttaccaccagtgtacctggaggtagatttggaaaaagactgttaaagcgtgtggacagtaaccagcgcagagt
acatccaacagaaccgccgataatgacggcaaaaagtgatttaatcatgtacccctcataaaataattttttatcgtaacaatatcagaatatgttctcattt
gactgatattgttaatttatatcctttgactacatgcaagataagggaaggttctatttattgacattaataccctcgacaattgctgagcagaagcccccac
gacaagtgacagaatgacaacatctacagtggggagcgttttatcgatgaacaagcaaccaagaagtaacacggcataggtgacgacaatgccagtaagcata
gaaataacaaaaacgtgagctttcatcattcctccagaataaccccgacgtgtaagcctgggggattggcacaataatgtcatttaaaggcagactttccagc
aaattgcgccgatgaacccagttcatcctcaattctcataagctggttatatttttcaatacgctcaccccggcatggagctcccgttttgagatgacctgcg
cgcattgcaactgtcatatcggcgataaagctgtccactgtctcaccactgcggtgtgagataaaggtgccccagttgttgtcatggcaaagttgtaccgcat
caaaggtttcactcagggaaccaatctggttgagtttaatcaatgctgaattggcaaggttttcgtcaatgccgcgctgaatatactttacgttggtcacaaa
cagatcgtcgccaaccaattctatctgctctcccaatgcaccatgcaggattttccatccagcccagtcatcttcagctaccgttaagggcaggaagatcaac
taggtgctctggggctggtgaccaatgcggttgtgctgtgaaacacactttacatgcaggaggcgttgtcacatcttcgcaaatcgggagaaactcctgagga
gtagcatccggcaagattatcgctgctgatacatagccatataaatatgctggggcattatacgtcttcgttaccggaagatattcttaagggtgagttaaga
tcattaaacttcaatacaaacaattaattaaactcttagcgtggttttttacatcactggccctcgccccccaattttctctatgttttgccactacttccct
cattttactgcgctgtttgctacccctgagaatcgacattacttgctatccagtccgcagcaactctctggcaacaaaacttatcagaatcataagtctgtgc
attattcgttcaagcgcatcgccaataagcggatgttgctggccgagtgttctgctaatcggatgataacaaatcccgtggttcggcattttcctttgtgcgt
gcgttgatgctggtaacggtaaacgtcatgatgaacatgaccgcgaataagacaacaattgtcggagctggcgcgctgtcgataaaaaaggacagccagacac
cagtcattgagaccaatatcgatatgccagtcgccagcaggagcgcaatatgaaagcgttgtgtcaggagtaccgcaatggcaccaggcgcaatgagaagaga
aatagagagaatgatacctaccgctttcagcgtcgccacaatggtcagggagaccatgcacagcagcccataatgcagccatctcgtatgcaaaccgcttgcc
tgcgcctgttgataatcaaagctgaacagcaaaaaatcccgccattttacgctaataacaagtgtaaccagtccggcaataatcaccgtctggattatatcgc
cgatggttatcccgagcatgtcgccgaaaagaatatgatcaagatgtacgtctggtttgactgcaatatacaagatcagacctgcggcaaacatgccagaaaa
aacaatccccatcactgtatcttgcttgattcggctgttgtctttcaggtatcccgtcgccaccgcacagaatactccggcgacgaatgcccctgtagccaga
ggtaaccccagaatccaggcaagcacgatgccaggaaaaaccgcatgactcatcgcgtcacccatcagtgcccaacttttcaggaccagaaagaccgacagca
gcgcacagggtatggaaactaccagtgaaatcagcagtgcgtgactcataaaggtgaactgcagtggctccagtaataatgccatcatgagcgatcctccagc
gctttatgcgcacgccgacgatttgccaaaagcccatgggtgggcgcaaagacaaatgctaaaagaaacagcagtgtctgcgccaccacgatgattccacctg
tcgcgccgtccagaaagtaactgacccacgcgccgaggaaactggtcacactgccaatagttacagcaatcatgagcaggcggggaaaacgatcggttaacaa
ccatgctgttgcaccgggagtcaccaccagacagatcaccagaaaggcaccaaccgtttgcagcgccgcaacggtcgaaaccgacagcagggtgaagaagagg
atttttaatctgcccggatgcaatccaatagcacgggcgtgattctcgtcgaaaaaggtcaccataagatctttccatttgaaaaaaagcacgatgatcgaga
gaatgccaatgatagtgagttgcaggatatccgccggatcgattgccagaatattgcccaggacgatggtctgaatgttcacggatgtcgggttcaatgacac
cataaacagccccaggccaaaaaaggacgagaagatcaggccgatgatggcatcttcttttaagcgagtgagttggttaagaaacagcatgctgcccgctgcc
agtccgccggaaaagaaggctcccagagaaaacggaagccccagcatatatgcccccgcaacacccgggacaatagagtgcgacagcgcatcgccaatcagcg
accagccttttagcatcagatagcatgacaggaaagcgcagagaccgccgaccatcgccgagacccacatcgcattgagcatatattcatagctgaagggttc
cagtagcacgttcatctttcttccctctgcaccgctgacggtcgatgtgccacaaaagggcgttcatcgtcagtaataatgctttcttccgagccgttaagtg
tgacatggcgcagtacgccgctaaaagccagctccaggttctcggctgtaaaagtggtgtctgtcggcccgcttgccaggacggtgcctttgaccatgaccgt
ataatcgcaaaatgtcgtgacggatccgagattgtgcgttgagacaagcatcgttttgccttccgcgcgtaactccctcaacaggctgatgattttcgcttcg
gttttcacatcaacgccggtaaaaggttcatccagcaggatcacatcaccctgctgtgcaatcgctctcgccagaaagacgcgtttcttttgtccaccggaaa
gctcgccgatttgtcgatggcgaaaatccaccatatcgacacgttccagcgcatcggtgacaatctgacgatccctctttttggcgatacgcaacatacccat
atgcccatagcggcccatcatcaccacatcttccaccagtacaggaaatgaccagtcaacctcttctgactgagggacgtaggcaaccaggtttttctgtagc
gcctgtcgtgtgggaatacccagaacagatatcttcccgctggtcagacgcacaaatcccataatcgctttaaacaacgtcgattttccggaaccgtttaccc
caaccagagcggcaatagatccgcctggtactgtgaaggatgcatcgcgcagtgctgtgtgcccgttacgccaggtgacagtaacatcattaacgacaatgcc
tgcagattgcatcattatttctccctttttccggctttaattccctgtaccagcgtactggtagtaaccttcagaaggtcgatatacgtaggtaccgggccgt
tttctgtgctcagggaatcgacatagagcacaccaccgtagtgcgcgccggtttcacgcgcaacctgacgcgctggtttatcggaaatcgtactctcgctaaa
gactgccgggatatgatttttcttaactatatcaacaaccttacgtacctgctgcggtgttccttgttgatcggcattaatcggccacagataaagctctttt
agccccaaatcgcgtgcgagataagaaaaagccccttcactggtgaccatccatcgctgattctcagggagttccgcaatctgcttacgcaggggggcaaggg
tttgggtaatcttggctttataagtatcggcattacgttggtaggtttgtgcatttgccggatcgtattttataaacgcatcacgaatattatcgacgtaaat
cagagcattatctggcgacatccaggcatgggggttaggtttgccctcatagggcccttcggtgatccctactggcgtcacacccgaagagacaattacttct
ggaaccccattgagatgctggtaaaagcgttggaaccacaattccagattcataccattggcgagaatcagttgtgccccctgcgcacgtttaatatcgccag
gggtaggctgatactcatgaatttctgcaccaggcttggttatggatgagacttctgcagcatctccagccacgtttttggccatatctgcgatgatggtaaa
agttgtaatgaccttgaatttttcagctgcactcgcctgaaatgctatcgagcaggtgaggactaggcaccccaagagcatggttactttttttattgagagc
atgatttagtacctatagtgagcaaaattagaataaacatagcaactactatgttttatagcagaagctataaaatgtaaataattatcattagcgattataa
gatcccaacacagggattaggttatttcctgctgacgatgtggtattgaggataaaagttgggcagcgctttggctttaaaatgaacttgttcaggacgattg
gtaatggcaaagcaaaaataatgtaggttattggcaagttctctaggacgcaggagagcatgaaaaacccaggggcagtcactaaatgaagggatcggtgatg
ctgccaacttactgatttagtgtatgatggtgtttttgaggtgctccagtggcttctgtttctatcagctgtccctcctgttcagctactgacggggtggtgc
gtaacggcaaaagcaccgccggacatcagcgctatctctgctctcactgccgtaaaacatggcaactgcagttcacttacaccgcttctcaacccggtacgca
ccagaaaatcattgatatggccatgaatggcgttggatgccgggtaactgcccgcattatgggcgttggcctcaacacgattttacgtcacttaaaaaactca
ggccgcagtcggtaacctcgcgcatacagccgggcagtgacgtcatcgtctgcgcggaaatggacgaacagtggggctatgtcggggctaaatcgcgccagcg
ctggctgttttacgcgtatgacagtctccggaagacggttgttgcgcacgtattcggtgaacgcactatggcgacgctggggcgtcttatgagcctgctgtca
ccctttgacgtggtgatatggatgacggatggctggccgctgtatgaatcccgcctgaagggaaagctgcacgtaatcagcaagcgatatacgcagcgaattg
agcggcataacctgaatctgaggcagcacctggcacggctgggacggaagtcgctgtcgttctcaaaatcggtggagctgcatgacaaagtcatcgggcatta
tctgaacataaaacactatcaataagttggagtcattacctgctttttcggttgaaaagaaattgattgaattactggaaaaacaatctggtctccatacgtc
tgtggattgcagtactccccttacagagcagagtctgtcgagaaatgacggtgaagaccagaccagaagaaacgctgaaaatatgttcggagatgactgaact
aacccagcctggccaggggaatatctcgccatttagttgtataacactgactcaacatttcccgtttcatttgccattcaggggcggttcctcgccccgcaaa
ccacactttcccttttcccgactggttcagttcatcgagtgttttcattaacttttcactgtttttacggggctggatttcatcaaataatcccggctgcgct
atacccgatggtgtgaaatcagccagcatgactcctgccttcatatagcggtacccttcacgccagacatggtttaaggctctgcatgcggcggcaataatgt
cccggctgtcctgtgtgggcaatgaaagcttttccacagcggcattgctgtaacagggttcagggggatgtccgtacaaatgtcgtcacctgccggcaatact
gacgctccccacgtagtttctctgcggcccgctctgcatactgaacaacagcctggtgcatggcatctttgtctgtgattcgttcaccaaaactgcgactatg
tcaacgccacgatgtttgaccgttatttgccattttcatgcccgctaaatactttcgttactgatgctgtccgctttgcaccatttcatcatcactgattggc
gttgtgtttttggtcagcactccagctttacgtttctcttttaaccggtaactctctccttgttctttttgacggacgcattttacgtttgggctggatgtag
taacgcagcatggaacgtccgcccgtataacccattgccttaatctccgcgaagataacctcgctattccagacattctctgccaggcgcatgtcgatgtaat
ccataaacggtttcagcttaaccattttgtggcgggtctttctggctggcggttcagggtatttgaggtagcgtctgaccgtccgttcagagcaaccaatctg
agccgcaatatcgacaatgtacgcgccctgctggcgcatttgctttatcatgtaaaagtcctctctgctcagcatgttgatgtcctttctggtgtgagaacct
taaggaaacaacatgttgggtggagcggacaatccaaatggtgaattaccgtcttatatcactggcgctgacacctgtagtttgtatacacaatccctggcat
tgaaacacacatatgaagtgtacttcggatatgaccgtacgctcaggaagtacggcagattccgtcaaaaattacctgtaaagacagacatcagaaaaaacag
tgctcgccgcagaccggtgaccgggcgaagcccgcgaaccggcgaggaagcgcaatgtatctgtagttacagggagcaggtacgccctgaagccctttcataa
aacggttgtgtgcgctgtccttctggaatgatttatccacatatccggaggcccgatccagtaaacagatccatgaatgatcaacaaaggatccattaaagat
ccccatatcgctgcaaaccttgtcactcatgggccgggaccacgatcacataagctgtggcatgttactgataaactgtaacatgctaatgataagctgtatt
cagtaatccatatactgaagtaagttaatgacataaactatggtcagtatgccagactcagttgttaaatacaggctgcaggtttttcttcagtcagttagcg
ggagttctgacacacgattttctgtttattcttttactgtccacaggctggaggctttctagaaaacgaaaattcagacatcaaaaaactgttcggcgaggtg
gataagtcctccggtggctggtgacactgacaccaaacaataacaacaccgtacaacctgtggcgctgatgcgtctgggcgtttttgtaccgacccttaaatc
actgaagaacagtaaaaaaaatacactgtcacgtactgatgccacggaagagctgacacgtctttccctggcccgtgctgagggattcgataaggttgagatc
accggcccccgcctggatatggataatgatttcaagacctgggtggggatcattcattcctttgcccgccataacgtgattggtgacaaagttgaactgcctt
ttgttgagtttgcaaaactgtgtggtataccttcaagccagtcatcccgcaggctgcgtgagcgcatcagcccttccctgaagcgcattgccggtaccgtgat
ttcgttttcccgtaccgatgagaagcacacccgggaatatatcacccatctggtacagtcagcttactacgatactgagcgggatattgtgcagttacaggcc
gatccccgcctgtttgaactgtaccagtttgacagaaaagtccttctccagcttaaggcgattaatgccctgaagcgacgggagtccgcccaggcactctaca
cctttatagagagcctgccccgggatccggcaccgatatcgctggcgcggctgcgtgcacgcctcaatctgaagtctcctgtattttcccagaaccagacggt
cagacgggcaatggagcagttgcgcgagattggatatcttgattacacggagatccagcgggggcggacaaaattcttctgtattcactaccggcgtccccgg
ttaaaagcgccgaatgacgagagtaaggaaaatccgttgcaaccttcacctgcggaaaaagtcagtccggagatggcggagaagcttgccctgcttgagaaac
tgggcatcacgctggatgacctggaaaaactcttcaaatcccgctgaacataaactgtagtcagtgaagagcgctcctttactgactacagcttatattaacc
ggtgcagtgagtggtctgctcactgcagtttatattctgtttcctgcagtgctgtctgtagctgagctgccatctgcctgtcccttacgtaagtgatcccgta
acctgatcctgaggcattgctcccttcataaaacatgacttactcactacagcttatatacatgctccagcttatgttatgtctgttctgctgaccacagctt
atataaggaagcgcgtatgccgtcacctcaggggaggcagtgtacgcaggatctccgcagcatcccgcccgtcacctgtgaaaggcactgccagcgtggcagc
catatccagtgcaaacactctggtataaacctccatcgaacgtggatccctgtgaccaaccagtgcctggatgactttccggggctggcggtgatagagcatg
tgcatgatatagctgtgccggaaggtgtgtggtgtgaccggaatcgaaaagtgtactccgtcagcttcggcccgtctgacagcctgcttcagccagttgcgca
tggtttcgtcggtcacggcccataatggttcacgacgacggggccgggtggtgatcatccagctttccatctgcctgacatagcttatatctgtcagcggaac
caggcgcacttcatcttttggcgggcgtccgcgtcgcgcacgcactttttcggacaggatccgcacaaacggtcttactccattcaggtcaaatgattccggc
gtcagcatccgggcttcgccaatacgcattccggtattccagagggtggcgaacagcatatggtgacgctgatccggcatatagaagagaagggcactcactt
ccggggccagcaggtaggccggtgtggcccccgtggaagtggccattcttctcagggagagcgctgttgcaaaatccacccccggcgcaatgggtaacaggga
gacgcgttcaggtgaattctgcaggggaatgacattgttcatgagttatttttcacgtagcacacgcactgtttatggatacaggtattctgacctgaacgcc
cggatccttccacgacaataaatcgcagcgcgcggatccttttgtttgattatcacgcattattgcgggtaaaaccaaatacataaaggcgcattttgccgct
gggatcccgtcttaagccagtcatggtgcgggctgagggctggtttaccgtttttgggagaattggggtaatgccaggacaggcagaaacggaaaaaaaatcc
tgcagacgcccttaaaagacaactgaaaatttctgactccatattcctctggtttcagactgcttaatcacatcatcaacctgatacataataaacagattct
gttcacagaatgaacaattaagtaaatggttagattatgcgaaaaagtacagatatttctcaccatcgttattgaataatatgtcttcacacaaacaatatta
atattttgagtgtaagccctgactcaaagaaagaaatgtctgttttcggaatcaaaaatatatagttatcaaaatacattaatttcaaatttgaaattaatgt
aaaacgatttagtaaatgttatgacattataaatctactcctccatgtttagaggagtatttgcagactggacgctcttatatagttacttacctttatacat
attctaacataagcatttaccaaccttacagggaacagaccatagtagtttatatttccattcaggtctgaatgcaccgatagaatatatcatctttttatta
ttatcctgacaataccttcgtgctttatctatattcagccacataaggacacttcccggacttagtttcatacaattctcatcattcagaactgcaccatttg
gaacgtcataataaacattacatgagctttcagatttcagtactatatcaatagcacacggtttattatcccagaatagcacatggccaaacataagttctcg
taagtgagaaataaacattaacagattatcatattcgtagcaaggtagggtacctccaaaccgggaatgaaaaagtgatatataagagctggatatttcttta
tcggataactggcttatacacttaatctctccgccattcctgataaatctctggatttcatttcggcgagttttttcaaatttaggtgaaaaagattccttta
taatacaattttgtttttttctggctattttccatgttgcattgataatattttgtttatttataattgataatttattagttttatccggaaaaaaacatct
tgcatgtggggaaaaaggaattaatacctcatcagatgaaagaggataagaccttcttgccattatcccaatttgttttccattgcacagaaaatatgagcct
ttgatatcaccatctttttcatagtgaaaaaaatcaaacttccagtcatgatgtatcataaagtatgaaacaacatccggatgcatgtttacactaccaccgt
atttttcaaaagtttttttgtacgtttcgaatgtagatggtttccaggccgtaataatgcgctttatatttttaattgacaaagtcatatcctcggcgttttt
atatgttatgagtgcaacaccaacaataattctgattatgataaatattatttaaaatcaacttccatttgttgttgaattgaaaggtgttttgtcgtttcta
tacagtatttaatataatcagtaaactttggcggtttaggcattccgatactcagtagcttgtcgttactgaaacaaacattgagcatactgaacgctccata
taaacgaatggcttttaacataaggcgttcgttacagggaccaaaaatatttttaaaatcatgacggctcattgccagtatgtcataactgactttagtatat
ctgtctccaacaggatcacagttcaaagcacgggctacagcttcgtcaatagcagagaacgtcacactactttctttacctgctgatatatgaacaatctcac
cattaattaacgaactttcaagcaacattagcaatgcatcagcacaataatctacagggataacatctattttatcatccagagagcacataaatttttgcaa
cattaaccccattctgaatacccagaaaatactggttgaaggtaagcaccctaaacgactgtggccaacaataattgatggtcgggcaaccaacaaaggtaaa
tcaggacactgcttacgcatcagatattctattgttgcttttgaatgcgtatactccactaaatgttcacctgtttctgatgaagcagactcttccttaacta
gcgaccccgtatgaggtgtacaagacatagcagtaccaacatgaaggaagcgtttcagtcctgccacttttgccattcttcttgcaaaagcaagtgtacctgt
aacattcacattccatataaaaggattattaccaaaagaagctatagccgcacagtttataacatgagtgacttcatcaagacgaggatccatgagaaaggct
tccggattatttagatccccaggcaagatgttatcattagttaatgcatgcaacctttcctcacaaacattaaatttacgcatattttctttaattctttcca
gtcccgcttgtggagtaggtgctcgtactaaaagtagcaaatttatattattacagttatccagcagcttatccaggaccgcgccaccaagaaatcctgttgc
acctgtaagtaataacaatttcatatctaacctcttttgttttcatgtaatcattataaaaataaattatcttacataaactatgattaaacaaaatcaagag
agattaaatgtaaaaacaatgtttatttttatacgacaggatccttcaggttattgcgagtgtttttattggatggtcttcttattattgacgagtaatagaa
ttataataaaattcatattattaacatcgcttatcaattatttttatgtcgataacactgtgtcgttatgcatgaagagtggcagagcagcatctttaaaaat
acaagagttaacagaaacacttttatcgttgagcacatataccctccacatattataaaaaatcaaaagaccaccatcagaagtgctggccttttgtttatca
tatttaaagtgtcatgaaaaagttaaaaacaggcatgcttcaggttaaaaataatacttcagaccagctgtggtcataaagttatagctttcaatgcctgcac
catttttggtgtgatcagatatattcaaatttctggagtacagagaagtatcacctttcttattagtaatacggttccatgttccttcgacataaactttagc
tgcaggtgtaatataataaccagcatgtagtgagacagagtaatagttctgattatttacatcgctacgataggtgatccttttctcagggttgtaatgctca
tcattatccgatgccttcacccatccgctatatttgaaactccccccgaactcaaagctatcataccgataattaccggttaaccctatatagggcattttaa
agtgttgtttgtagcctatagctctttcaccatctggaaatgatcccgtttcatcccggaagcctccctcactactatagatataagagcctccttttgctgt
aaagctataacgattttcctggtaaccagccataagacctaactgatagtctggttgattcaggagccatcccttaatattcaggtcgaattcattagcaaaa
tttagacgggtattcggatgtttactctcatcagtccaggttccgggattgctcgtatccagccagtccctgtccaccatattgcctccacgaccagctaatg
ttgtccaccctgaggcaccgacagatactctgggtaacagatcccagttaaatgcccccttgacaattggcgcattactgtatttccagtccagctggctggc
tttgcgccctttttcttcaggcagataaacccgttctttggccttgccacttaaagtgccaaaatcaatctccgtactgattttctccggtgtaaaagaaagc
ccagtatcagaagccagtgcagcaaacgctacaggagctgagagtgcagtagcaagaatctttaagtacatagacaagttaactccattccactcgtttgttt
ttgataaatatttttttatttactcgtagtagtcaactgaaaaatgctgttttgtttaacgtttgtttacaatgtggccgcatgtcgctttatttattccctt
ttatgtcagaatatcaggggagtcagtcaaacccggtatgattactgccccggtcatccatgttccaggaaatatcagggcatacagggtgctggtaactgcg
aataaggccctggtcggacactttatgccgggaacgtctgtaacatggaaaccggtaccttcttcctgggtttattttgttaactcttatctaaagggagaca
aaatggccaaatcctgcgattatgaagcaatcagggtttttattagcgttaatgtcggcaaatataccaataacgccattacagttaattattcagatagcgt
ttgtgcgacaaggcattaccattcgacgaaaataaactcaggtagttgatatcagaaatcaaccgtggctccacctacaaaaaaagttgctggtttataaatc
cctcctttctgattaataagaaaactaataacatatgagtataattacttgttcagataagttaattatcttcgtgattgatatttatttggtttcagtaaaa
atgatgtttttattataaagtttataaataccaagtcggtggtgtttattagtgtaatggaggggagtgcgtaaatcgttacatgtaacacttccttccaata
atactttataaatcaataaatgtatgattacttatttcctgaaatatcacatatattgctatatttcttaacgagatattcttattttattgatgattgcatc
tcttttatttaatgtaaagctttcttattaatcttctaatgtattgcaaaatggtagatgctgagtaatcaacagacttgtatgtaaagtcaagattaacgtt
ctaaagcacgcatcaattttctgataaagaggatgtggttgtttttttcgataaaaaaaattcttgtctaaccgctggtgaaataagattattattaccccgg
attaatactgagcctaacaatagaatacaaaaaaaacaaaatgctttaaataaggggagcactataagttatcctctttactttatgcttaccaactcatttt
gttactttataacataactcatcttgcgttgtaaacagactcgttttagttccatgcaccttttgagatcgtgaccaaataatggcgttagtgatgttcttcc
agtgtcatatggttaagtggttgatatggcgttcactgttatattctgacaaccagtttgccgccagttcccgcttttcatccagcgttctgaacagacaaaa
accgagtatttttgttcggtatgtccggttaaaactctcgataaaagcgttctgcgccaacttacctgattgggtaatctccagttttactacatgtttttct
gcgcattacgtccgtgttggataaaaaaaatcctggccataatctatgcgaatcatagctggatagacatggttttccgcaatccttgaaccgccccgggaat
cctggagactaaactccctgagaaagaggtaaacaggatgactaaaaatactcgtttttcccccgaagtccgtcagagggcgattcgtatggttctggaaagt
cagggcgaatatgactcacagtgggcggcaatttgttccattgccccaaagattggctgtacaccggagactctgcgtgtctgggtacgccagcatgagcggg
ataccggaggcggtgatggcgggctcaccaccgctgaacgtcagcgtctgaaagagctggaacgtgaaaatcgtgaactgcgccgcagtaacgatatccttcg
tccaggcttccgcttattttgcgaaggcggagttcgaccgcctctggaaaaaatgatgccactgctggataagcgcgtgagcagtacggggtcggaccgctat
gcagcgaactgcatattgccccgtcaacgtattaccactgtcagcaacagcgacatcatccggataaacgcagtgcccgtgcgcagcgcgatgactggctgaa
gagagagatacagcgcgtatacgatgtacggtgtgcgtaaagtctggcgtcagttgttacgggaaggaatcagggtggccagatgtacagtggcgcgcctcat
ggcggttatgggactttccggtgttctccggggtaaaaaggtccgcactaccgtcagccggaaagccgttgtcgcaggcgaccgcgtaaaccgtcagttcgtg
gcagaacgtcctgaccagttgtgggtggctgattctacttacgtcagcacatggcagggggtcgtctatgtggcgttcatcattgatgtgtttgccggataca
tcgtggggtggcgggtctcatcgtctatggaaacgacatttgtgctggatgctctggagcaggcgttatgggcccgtcgaccgtccggcacagtccatcacag
tgataaaggttctcagtatgtatcgctggcctacacacagcggcttaaggaagccggattactggcatcaacaggaagtacaggcgactcgtatgacaacgcg
atggcggagagcatcaatggcctttacaaagcggaggtaatacaccgtaagagctggaaaaaccgtgcagaagtggaactggccacactcacgtgggtggact
ggtataacaatcgacgattgctggaaaggctgggccatactcctccggcagaagcagaaaaagcttattatgcttccatcggaaacgatgatctggcagcctg
agttcacagataaaacactctccaggaaacccggggcggttcacctacagagtaccgctaccacttgtgaagctggcagattcagattgttttcaatcgacaa
cgcctcatagttaaagtcatcaacgacattgaacgtgtgaagtggtcaacaaaaactggccaccgagttagagtttttccagtatcgattttccgattcgttt
ggggataacccaccgttatattcgtgcggtcttagtgcgctgtaatatccaacgatatagaccgttatggcgtgagcggcctcgcagaagcttcgtaacccac
caccggcatccattcgttcttcagactcctgaagaagcgttccattgggctgttatcccagcagtttccgcgccggctcatactctgcctgatctggtatcgc
cacaataactgccggaactgcctgctcgtatcgctgtggaacatcaccccgccgggcttacccgtccagatataggtcacatcaccgcaccacacctgtatag
cggcgtaatccccaaaaacgatgctggttgtcagtaaacaagccatgaatgacagtcgttcctgaaatcataagacctggaggagtgtatgctccgtcgcagt
aattaccacgatacgcttgcagaattttttccagttactgaagcgtgaacggataaagaaaaagctcttcggaacgcgggaagacgcctccaacgatattttt
gaacatcgaaatgttttataacaataagcgtcggcatggttcctgcgaacagatgtcaacaatagaatataaaaacaattattatcaacaacttggaaggagt
aagactactcgtggcgattcaatattagatcattgatggagatataaatggctcactttttatcggttgttgacgatacgtttatcaagtatacatgaaaatt
tcttcataccagctatatgagacgttaagactaataatcttactcccaaaatgactatgttgttgcagtttggtttattgactcttgtctatagttcaccgaa
tgccaagatgagggagcgaggaactttactgtctacgcctctcttcctggttacaaagacaagcgtaaaactcaccatccattagaactaaaggcactttcct
gagcagagttttcatctgcaagataaaagctattttacagccgtaaagtccagtgaaacgaaagagaggttagtgaattaattacttgatgtattaaagtata
atgggcgtagataacaccttaatacgaaatcttgtcaggctttcccggtaattagtaatactgttgaaacaaagcgagagggggttagcccaatttacgcaaa
taaataataatcttgattatgattaattccaaataagtatatttttccagtagatgctgggcattaactaagaaaacccagaataccactttcattgttatgg
aaaacatggacaatgttaatgaaatatatttctcaccgcgccatcatttgtaccttgacactgttgatcattattatcaccgttttgttcacctttctgagaa
gctctgatgtgccagaatatattactgccccggtgcgcaaaggcgatatagaaaattctgttctggcgacaggccgtattgatgcaattgaacgggtaaacgt
aggggcacaggtatcagggcaattaaaatcactgaaagtgaaacagggcgaccatgtgactaaagggcagttgattgccgaaattgacgacctacctcagtgt
aacgatttgcgtaatgccgaagctgcactcaacgaagtgaaagcagaacttcaatccaagcaggcgctcctgaaacaggctgaactgcgttttaaacgccagc
tccggatgctaagagaaaatgccagctcacacgaagattttgagtctgcggaggcaatgcttgcgaccactcgcgctgaattacattctctgaatgccaaact
ggtccaggcacagatagaagtagataagaaaaagcttgcccttgaatatacgcgagtcgtagcgccaatggatggaattgttattgccattgtcactcagcaa
gggcaaaccgttaactcaaaccagagtgccccaacaattattaaactcgctcgtctggatgtcatgaccattaaagcacagatctccgaggctgatataaccc
gtatttctgtggggcaaaaagcccgcttctctatcttctccgaaccggacaagcactacagcgcaacactgcgtgccgttgaactggcaccagaatcggtaat
gaaagatgactctctcgccagcaacacttcagcatcaggctccggaacttcaaatgcgtccgtttattacaatgctctattcgatgtgcctaacccagaaaat
cgacttcgtattgccatgactgctcaggttaccctgattactgatgaagcccaaaatacactgctggtacccattcaggcagtgcacagaaatgaggggaaga
agcaacaggtcctggtgctggcagcagatggcaggctggaaccccggaatgtgaaaactggtatcacgaacagcgtggatattcagatcctcgaaggtttaaa
tgtcggagaaaatgtcgtgctgtcgctgccggataagaaagagcccgaagaaaggattatgctgtgaaaaaacttattgagttaaaaggggttagccgaactt
atggcaacggagatcaaacgcggaccgtcctgaaaaatgtcgatttgacaatcgttgctggagaaatggtggcaatcattggagcctctggctcaggcaaatc
aaccttaatgaacataatgggttgccttgacgtacccaatagaggtgattactacatcgacggacaaaatgccgcctgtctttcacccgatgagctggctaga
gtgcgccgggaacacattggctttatattccagcgctaccatttgatacctgatcttagcgcacttggtaacgtggagatcccggccatttacgccaacagcg
aacgcgacagccgtcggcaacgtgcaacggcgttgctgggcagactgggactggaaggacgtgagcatcataaaccttgcgaactttcaggcggtcagcagca
acgcgtcagtattgcccgagcactgatcaacggtggaaagataattttggcggatgagccaactggtgctctggattcgcaatccgggcaagaagtgttagcc
attctgaatgaacttaatcgacgtggtcatactgtagtaatggtgacccacgatatgaaggtcgcccggcacgcaaagcgaatcattgagctgtgtgatggcg
aaatcatcgctgatagtggaggttgcgtatctgcgacggaaacactaccaaaaaccaacagaatccggcaaagctattggaaaacactgctcgatcgaacgcg
tgaatcgatgcaaatggcgctaaaagcgatgaagacgcatcgcttacgcaccacgcttaccatgatcgggattgtttttggtatcgcctccgttgtcactgtt
gtggcattgggtgaaggagccaggcaagaaacactggaagagattaaaagtctggggacaaacgtggtcagcatttatcccgggcaggatttgttcgatgaca
gcattgagagtattagaacattggtacctgctgacgcaaatgcactggcgaagcaagggtttatcgatagcgtcagcccggaagttagcgcttcagacaacat
tcgttttctgggtaaatctgcgatagcgtccattaacggcgttggggggaacatttccgggtaaaaggtattgagttgttgcaagggactacttttagagacg
atcgcaacgctctacaggaagttattatcgatgagaatactcgtaaggccattttcgacaacacagggttacaggctctggggcaaatagtctttcttggttc
tgtaccagcacgagtggttggcatcgctaaaagtaacaaccgtagtgatgcatcaaatcgcattactgtgtggatgccttacagcacggtgatgtaccgtatt
gtgggtaaaccagttctgaccggtattagcgtcaggctaaaagacaatgtggataacgaggctgcaatcagcgctatctcccaactattaacccggcgtcacg
gcataaaagacttccagctttataactttgaacaaataagaaagtctatcgaacacacctcaatgaccttcagtattttaattcttatggtcgcatgtatctc
actaatgatcggcagtatcggggtgatgaacatcatgttgatttctgtaacggaacgaactcatgaaattggcgtacgaatggcggttggcgcgagacgcagc
gacattatgcaacagtttattattgaggccgtattggtttgcctgattggtggcgcactcggtattgcactgtcgtatatcacaggtgcgctctttaacgctc
tggcggacgggatattcgcagctatatattcgtggcaggctgctgttgctgcatttttttgctcaaccttaattggcataatcttcgggtatctcccagcccg
caaggcagcaaggatggacccggttatttcactggccagcgaataatatatgaaaattttatcatcgttgactttatgccttatcaccgcactctgttcaggt
tgcacaaacgtgctgaaaagtgactatcgcgctcccgaagtgaactatcccatcaactggaccaagggcgatgtggacggaaatacatccccgtttgactggg
aagaatttaacgatcctaatctcgataactggcttcacctggtgatgacaagtaataacgacatagctatagctgcactgcggatacatcgggcgcagctgga
tgcggaaagaacaggtatcaccaatactcctgcactaaaggcggcactgagtatggacggaaaaaaacagctgaataactcgtccggctgggcaaaaagcggt
tctgccagccttggcaccagctatgagctggatctctggggaaaaattgcccgtcagcgtgatgttgctgagtgggcagtccatgccagtgaagaagattttc
gctccgcgcgcctgatgctgctttcagaggccagcaataattactggcgtatcggttttgtaaatcaacaaattaccaccctccagcagagtattgactatgc
aaaagagacgctgcgtctggccgaggttcgctatcgtgcaggaaatatctcgtcgctggatgtgattgacgcgcagcaaaacctgctcacgcaagagaaccag
cttacagggctacaacgagaacattcacagctactcaatcagcaggccgttttgctcggcaccgtgcccggttgccagatagtcgagcccacaacgctgccaa
aaggaagcctgcctaaggtcaatgcgaatattcctgccagcatactgatgcgccggcctgacataagtgctaaagagtggcagttgcgtgaggcgctagctac
ggttgatatcaagcgtagcgaatattaccccaccttcaacctgacaggcgctttgggtaccagcagcgcttcgctactggcattactgcataatccggtcggt
tccgtaggcgctaacctgacgctaccgttcctggagtggcgacagcgggacatagaggtaaaaattgcccgtaacgattacgaacagcgagtgctggagttta
aacagctactttataaggcaatgagcagtatagaggacgcgctttcatttcgtaaccagttgctgcttcaggagacaagactcagggaagaactggaacttgc
acgtaagtcagagtggttaaacgaagttcgttaccggcatggtgcagtacgtatttcattctggcttgatgctcaggaaaaacgtcgtcaggcggagctgcga
ctggatgaaaaccgcttcaatcagttgcagaatctagcaaaaatttatcttgagtttggcggggcatcaacctttccttaagtctaattggggacaaccagga
gcaataaggagtgaaaaatagccacagctgaaaacaccatagccattgacatttgagctttacctgctcttgaaaacaaaaaatcactgttaagcgcaatgct
attcacttcagcagagcagtattgcgtgtcacgagcatgtttaccattctgtgtaaatatcttttcttaaaagtgatcatcaaagcagtcatcaaactcgata
aatatccaccggcatagcgtaatgccgagcaattaaggatcaattgaccgctccttaaactgcggcactataacggcttctacaacagggagccgttttcttg
tgcatagaaaacccccagctaggctgggggttccggaaagctttcagctttgagccagttattaaaaccccttttgatttattaaaacaccttgcggtctggc
aactgcaagtgtcaaacaagaaatcaaaagggggtcccaatggggaacgaaaagagcttagcgcacacccgatggaactgtaaatatcacatagtatttgcgc
caaaataccgaagacaggtgttctacagagagaagcgtagagcaataggctgtattttgagaaagctgtgtgagtggaaaagtgtacggattctggaagctga
atgctgtgcagatcatatccatatgctacaatgtttgtcaaagctcagtgaaaatgtctaatcactggctcattataaatgtccatatattgaaaaggccgct
gcgaaaatccctcccgcagcggcctctttagcataacgtcattatgcgaaccatcacttaatgttcttctcgcattttaagagaaatagtaaatttgtagcag
taagatgaagcatatagcttaccaattcttccggaacctctttaatatcagctccttgcccatgacctgcatttttgtttctgatggttggaattcctgtttc
cagcattgttttcagacttgtgaactggctttgtaaatatgccggaattaaattttgacttaaacagctatttattagttttgatgctgtatcattaggacta
tatttccagttattcttatcatgtattgctttcattatactttcaaatgatttcagacaatcatttagacattctttataacgcttatgcctgtaatgctcat
gcgcagcaaggaattcatcattggcgccttcaaacaagggttctccacttaaaagaatcagggttggctttacgacatcagcatgaatcaattgagagtctat
ccttattatctcttcagattcaaactgataaccaaccccatgttctctaaacctttcatttagttcaataactgcatctccaggactttgacttgttatgtct
ttgaattcataatggtttttcgcaacgtggctcactagtatctggaagcatatttctatgaaatctaggcatttctctgtatttctctctttaaggaaaaagt
cgacaagagcgtcaaagtcattctttgcaaattccttcaaagaaaaaacaccatattccttagaaagtatctcatggatatatgcatatactttgtcagcttc
atttctatacgatgtgtagcgttcattagaagaggggaagccgatagaatcagttattattttaataatttgcaccctgagtgcatgtggaatattgttatat
tgatatacgtcatttacttcgccacgaatttttctttgtctcttagaataaatattaataattgccatgaaatagcctcaagatttttttacatgctgttctg
tgatacttgctgtttggctctttgcaccgcgaagcggttcggggttctgtgacacccgaattttacctcggtttcctttgtatataatatcattgaagttatt
tgcatttctcagaaatcttctctggctttgattgcaatagccgaaagatagcgcacacatcgccgttggtcagttagtacgaggtttccgccaagttcacctt
ttaccaaagatggcctcagagcttccctgaagctttatcatggcttcaagtaggtcatcttcgttcagtgagcgctgagatttgttgatgctaggggcgtgcc
gtctgcgggaaggctcatcaccagaacaaggtaacgattgtgagcgattattgtcccgttttctctggacctgacgggcaacatccaacacatggccaagtct
cttgttatctacaatggcgacgggatcaacctcagaaagccggtcataggtggagtagggcagcagcacgccatttagtcgcagctccttatgtccgtcagga
taatgccagacatcaatatacttgccgatggctcttcgactgtactcgttgtcctcaatcagatagagcattttatcgtattgtacagtcagattttttgata
cccggcgaggctctcgctaggtaaattctgcatcaaggtcatcgtctaatgccagcggccggtgcgcgttaaaatcatgcaagggttctttggcaaagcgccg
gttataatccttctgcaaatgcattggccgcttctattgtacagatccccttaagcctgagctccttaacgagtcggtcctgaagtgtcaaatgagcacgttc
aacgcggccttttgcctggctggtttcagcacacaaaggagtaatattaaggcagcgcatggccctggcaaactgggtttcgccggggccggtggtggcgtgc
ttattattgactctgaatacgctggctttatcgctgtagaggatcatcggtttaccgtatttctcgatatagccacgtgtagcttcaaaataggtaaatgtgg
actcagatttcacaaacagcaggtgcatcaggcgactggtggcatcgtcaacatagaccagggcagaacagggtcgaccccggttttcaaaccagtggtggtc
gcagccatcaatttgtatcagctcgccagtacaaggacgccggtaacgaggctgatgaattttcggtgcgcgttgtctgcggggaatccagagtccggccttt
atcatgagacgtcggatggtttcctttccaaggaccagaccgtgaacttcctccagcttttcccgcgctagcgtggggccaaaatcacggtagtgctcccgga
ttatactgagtgcctgatctgtcagtgacgctggcaacagacggttaccagtgcggccacgactctggttgttcatgccaagcggcccatactggcgataacg
tttgagcagacggctgcaatgacgtggcgtgatgcccagcatttcggcagcctgtccaggacggatattgcgatcgatgacatcctgaaggattttgaggcga
ttgatttctttcacggtgaacattccggcgtcatttatagccatagaaaactcctcatcaggaaaaagtgagtcgttgtctagacatagggatctgagaaact
atagcctggacattcctaatgagccacaaatagacattagcattgagcctctacaatgtttgtgcgcacaatctatcttatgttaaataacattgaatattaa
cttacttcatgaagcttactgcttctatttccacttctttttcttattaatcgctcccctaatccacgcattcatctgctctacaggctctaaaatatttttt
ctgagtactgagcacaaactgctccataaaaaacagaccacaagtttgggcatgatactgatgtagtcacgttttattgttttcatgaagctctctgctattc
cattactcaccggactccgcaccgccgtgttcttcgatttaagtcccaacatccgagcgaactggcgtgtttcattaacccggtagtcagagtcagcggtcga
aagacctcccaggtagctactctgcatacccactataatgcaactattctcgtgacgtgtgtttcagctctcccccggctatgatacgagatggtatggagag
cccttctggggccactgcctgcaccagatgcaacaggaagcctgatatgaatgcaatttctgtcaggacaggcgggtaactttctgtggccatggatcaggga
atactccacccgacatccaccactgaatttcttcttcagtcaacaagcaaagggataaggcatgtgtaaaatcattaacttcagtggcattgccaatgatagt
caactgacagcagcgatcgccaaatcgcccagaggctgtgtcgatgtgtcgctgtagcgctgaacgttcttcacgagttaaattattgtcagtatgttccagt
actccagctttccagtagccaatgagcgccagactgatgcttcctgctgactgattccagagcagggcaagatcatctctcccggggagccaaaaaaaacctt
tactgcgataaacgcccatgcccataaagcggtgacaggtatcccataatctttgcggatggaatggcctgtcgtccctgatgacgcgccatataagttcctc
attgttgcacggagtatccatgtttcctacaagtgcttccagttcactgatgagtaactcaacacggtcaaaattgtagtccgggatcgccagtaatgtgctc
agttcaatatttccccatgataccgcgagcacatccacggaggggttaagtgggtgtatggcttttgccacagcggagataatgtcaaagggcaggcggtcat
ttctggtcagcagcagcctgttgcagaacatgatttgctcggccagcaaattctccaggccgcgtttgttatgccggagattttcctgaagagtggggatcag
ttttttcccatcattgtaatcttcatggagcatgacagtatcaacgagtgttaaaaacgccttcaggcgtacacgtgaatggcgacgcaggtatttgaccaga
ggaagcggatgactgcttccggaggtttcaatcagcatcacatccggcttgttgtgatcaagcaggtggttaattgcgagatccatctttttgaggcctgcgg
ggctgctgatgctgtctgcggtgatacttacgaaattattgtcttttgcatctacaatatctgtgttggcgatcagcacgccgtccacatccagttcgctcat
gtcattgactatgacagagactgaaagctgtttttcgtgtgcctgtatgagcaggtttttcagcagagtggttttccctgcgccgaggaagccattgagaatg
atgacagatgttgtttccatatcagcagtacaccagttagttagcgaggaaggcgcttggtggtacaaagagcatccggattgaatcgcataacgtcgacggg
agttgttgtcgtttttcctgtgaggtctcaacctgccacagaacgcaccggatttattggtatttagtctccatcagcataattatcaccatttttatctccg
ggggaagatttgttctgaacaggagtgggaggtgtgacctcctgaaagtaattacagattgctagttgttgtatttttattcgaatatgtctcttcgtatcag
tgatagcacctagataaagtatattccgggagcgttatctgttctagatatcagagagtcagtcatttgtaaaaaagagagctgcatcattctatttttcctg
tctattcaccctgttgtcgtatctaaaagacatccgttccgaggatgtcgggcacccgttctgtactaagatacatagcttttgggtctgcactatttggaac
cgacagaagtctgtctgtgatatccgcatttttctgtgtgcgatatattccgaaaaacttcaaaccacttaaaagaggtgtttccggaggataagttcgacca
gggaatagtgtttcctggtaagtgtggcggagccagttcaccaggagtatctttcttatccagttatactggattttattagatgatcaccctaccctgcagc
agtacgctgacggtgtttttttgggagaaccattatgaataacgtcatctttgattggtatttatctaggcaaaaattcattcaacgtccactgccaagatgc
aacaggcagagtccttaactgtcagaatccaagtgctcgaaattattcgaattccttactaattcctcctacacaactattgttatggaagcttgcgaagatg
ctcactatatggcacggaaattttcggaaatgcgtcataaaacaaagctaatacctcctcattttgtccaacccttcgttaaaattaataaaaatgatttctt
tgaggctgaaaccatatgtgaagttacatcaagccatcaatgagatttgtagcattaggagctgaatcccagcagaggattccacttttcatccgtcgttgac
aatggccttgaaaaacgccgttactcaccgttctccttcacggtaatacgcagttatagttgcagagcaatgtaatggatcttttatattaagaaattgtgtg
agtggagaggtgcatagatttgcccctatatttccagacacctgttatcacttaacccattacaagcccgctgccgtagatattcccgtggcgagcgataacc
cagtgcactatgcggatgccattcgttataatgctcgaacgcctctgcaaggttctttgctgccgttaacccgtctggtttgggcatgacactgatgtagtca
cgctttatcgttttcacgaagctctctgctatgccgttactctccggactccgcaccgccgtgctcttcggttcaagccccaacatccgggcaaactgccgtg
tttcattagcccggtagcatgaaccattatccgtcagccactccactggagacgccggaagctcgttgccgaagcggcgttccaccgctcccagcatgacgtc
ctgtactgtttcactgttgaagccgccggtagtgaccgcccagtgcagtgcctcacgatcacagcagtccagcgcgaacgtgacacgcagtctctctccgtta
tcacagcagaactcgaacccgtcagagcaccatcgctgattgctttctttcacggccactctgcctgtatgtgcccgtttcgatggcggtacagcaggttttc
gctcaagcaacagcgcattctggcgcatgatccggtaaacacgtttggcattgatcgcaggcataccatcaagttttgcctgtctgcgaagcagcgcccatac
ccgacgataaccatacgtgggcagctctctgagcttacccagcaatagtggacacgcggctaagtgagtaaactctcagtcagaggtgactcacatgacaaaa
acagtatcaaccagtaaaaaaccccgtaaacagcattcgcctgaatttcgcagtgaagccctgaagcttgctgaacgcatcggtgttactgccgcagcccgtg
aactcagcctgtatgaatcacagctctacaactggcgcagtaaacagcaaaatcagcagacgtcttctgaacgtgaactggagatgtctaccgagattgcacg
tctcaaacgccagctggcagaacgggatgaagagctggctatcctccaaaaggccgcgacatacttcgcgaagcgcctgaaatgaagtatgtctttattgaaa
aacatcaggctgagttcagcatcaaagcaatgtgccgcgtgctccgggtggcccgcagcggctggtatacgtggtgtcagcggcggacaaggataagcacgcg
tcagcagttccgccaacactgcgacagcgttgtcctcgcggcttttacccggtcaaaacagcgttacggtgccccacgcctgacggatgaactgcgtgctcag
ggttacccctttaacgtaaaaaccgtggcggcaagcctgcgccgtcagggactgatggcaaaggcctcccggaagttcagcccggtcagctaccgcgcacacg
gcctgcctgtgtcagaaaatctgttggagcaggatttttacgccagtggcccgaaccagaagtgggcaggagacatcacgtacttacgtacagatgaaggctg
gctgtatctggcagtggtcattgacctgtggtcacgtgccgttattggctggtcaatgtcgccacgcatgacggcgcaactggcctgcgatgccctgcagatg
gcgctgtggcggcgtaagaggccccggaacgttatcgttcacacggaccgtggaggccagtactgttcagcagattatcaggcgcaactgaagcggcataatc
tgcgtggaagtatgagcgcaaaaggttgctgctacgataatgcctgcgtggaaagcttctttcattcgctgaaagtggaatgtatccatggagaacactttat
cagccgggaaataatgcgggcaacggtgtttaattatatcgaatgtgattacaatcggtggcggcggcacagttggtgtggcggcctcagtccggaacaattt
gaaaaccagaacctcgcttaggcctgtgtccatattacgtgggtaggatcactccgataacatggtgtatacggagaagcacatccgtatcatcagtgtgacg
actgcggcggccatccatccagtcatcggttcgtctgagaatgacgtgcaactgcgcacgcgacacccggagacaacggctgactaagcttactccccatccc
cgggcaataagggcgcgtgcgctatccacttttttgcccgtccatattcaacggcttctttgaggagttcattttccatcgttttcttgccgagcaggcgctg
gagttctttaatctgcttcatggcggcagcaagttcagaggcaggaacaacctgttctccggcggcgacagcagtaagacttccttcctggtattgcttacgc
cagagaaataactggctggctgctacaccatgttgccgggcaacgagggagaccgtcatccccggttcaaagctctgctgaacaattgcgatcttttcctgtg
tggtacgccgtctgcgtttctccggccctaagacatcaatcatctgttctccaatgactagtctaaaaactagtattaagactatcacttatttaagtgatac
tggttgtctggagattcagggggccagtctagtgcacgaatcctagaggcagaatgtttctccacatgcttgtggagaaaccgccgcattttgcccgttctgt
acgatactccgtagccagagataagatattctgttgctgtatgctacatacattacactatacgaaatcaggtctgttcaatcagtaaggaaataaacggaca
aacgttatgtaacccagacagagatatattgtctgggaaaatagtggcttttgtggcgaaaactagtaaaacagcaacccgaaccacttgatgtgcatcgttt
ttgattattcccgtatactcttgcagaaggagttctccgtcgggctactgtcatggttaatgcggggaatatggcgacaatacaacacacctaaaagagtaat
ggacagatgaagcggtttattcatttcccatgattctgagtacctaccaagtctgagtaaccacttttatacttttaattttcgttcatttagctatcgttta
attattatcacataggattctgccgtttttaacaatgcaggataataagatgaaaaaaatgttattttctgccgctctggcaatgcttattacaggatgtgct
caacaaacgtttactgttggaaacaaaccgacagcagtaacaccaaaggaaaccatcactcatcatttcttcgtttcgggaattggacaagagaaaactgttg
atgcagccaaaatttgtggcggtgcagaaaatgttgttaaaacagaaactcagcaaacattcgtaaatggattgctcggttttatcacttttggcatctatac
tccgctggaagcccgggtatattgctcacaatagttgcccatcgatatggggagctcatctgcactgttcattaatatacttctgggctccctacagttgttt
ttgcatagtgataagcctctctctgagggaggaaataatcctgttcagcgatgtctgccagtcgggggggctgcattatccacgcccgaggcggtggtggctt
cacgcggggatgggcagattgatctgatatgcaaccgacgacgaccagcggcaacatcatcacgcagagcttcattttcagatttgggccaccttttgatttc
tcgtttaacttttgcagtcaccagactgtgatttattttaacaaatcaatagctttcataaaattctcgaagctgaaagcttaactgaaccaaccagcctggc
tggtgttttctaaagacaaaaaggccatcagcaaaacactgatagtctgaatcacctttatcaatcatgtatgaagaaaatatgcgcaattagttgacagtga
ttatcattttcattaaaaagagcgcgtagcattcttatttcatgaggaaatttacccgccaacaacctgagtagcgaaagctgttcatccccaatatttttgc
tggcgggttcttttttctactgtcctcctccccagaggaagcattcccattaatcattactggcgaggaggcaagagaaaaagtcggttctttgtccctaaat
aacagcatcatttcagtccatgcaaaatctgatttaccagctcacataataacctgatgatacacagtgtaagttcacagagatattgcaattgcctccggat
aagtaaggggagattgcactatgcaaatgcagcacctgaggttggctatcctaagcagccgattatgcgttgaggctttcagtgatgggatatagcaacaatg
agaatgaaggctctgcacttctgggataaacacggtatttctgcagcttctgaagcgtttggcgtatcctgccgcacgctttactggtggcgtcagttactga
acaagggaggacctgaggggctaatcccgcacagtaaggcacctctggtgcgacgaaaaaagcactggcatcccgatgtgctgaaagagattcgacgcctcag
gacagagctgccgaacctcggtaaagagcagatttttgttcgcctgaagccctggtgcgcataacgccatcagacctgcccgagtgtttccaccatcggcaga
atgatcgccaccgcacacgataaaatgcggatgataccagtacgtctgagctcgcggggtaaggctctgcttgtcaaaaagcgaaccaccaaacccccgcagg
ccaaagcactaccgtccggtaaagacaggagagctcattgggatggacgccattgagctcagaatgggtgaaatgcgtcgctatatcattaccatgatcgacg
aacacagcgattacgtactggcgctggctgtgccgtcgctcaacagtgatatcgtcaatcactttttcagccgtgcagcccggctgttccctgtcggtatcag
ccagatcatcacagataacgggaaagagttcctgggaagctttgacaaaacgctgcaggaagccgccatcaaacacctctggacctatccctacacgccaaaa
atgaacgctatctgtgaacgttttaaccgggcattaagagagcaattcattgaatttaatgaaattttactctttgaggatcgcacctgagg
Nucleic acid sequence of pAPEC-2 ΔAMR plasmid (SEQ ID NO: 2):
gtgcgaatgcctccggcgcgggccggattattctgaggagatcactttcagggagaagctgtggccagccggctgtaattacggttacgtgacaga
atcatgcgctccttcacacgacgctccacttcgcgttttaccgcctcaccattagcagtgaagcgtccttccgagatttcacgcgtcagctgccgtttcac
cagggtgacgatatcctgacgttccctgttcgcatcacgatgcgcacgggcacgttttattccacgggacttaagctctgtctggtaactgcggaaacg
ctcacgcacaaaacgccaggctttcgctatcagctcatccatacccagggtatccagcccctgctttttgcgctgtttgttttcccattcaacacgactgc
ggcgcgcagctgccactgcatcctcagacacatcaagggcagcaaacagagccagtgtgaacgtgatgtcggtcggaatgtagcacccgataa
gcgggtcatattccgtctggtaggtaatcaaccccagctctgacaggaacgtcagggcacgggtggcacgggtgatggagagttttccggcagca
gactccgtcgccagtccgcactcaatggccagcgtggtgatggagcactggacgcggttggccagcgggtcatagtggaaacacagcccctgca
ggagcgcatcaatagcccgtcgacgcagcaccggtggcatgcgtcgacgcagaccacgcgaacgggcatgcgccacatgaatggcgaaatca
aaacgggaagtgaagcccaccgccttttccatcagtttttcgcagaacttcagcgttccggcacctttacggggtgtaaacaccggattcgggttcttta
cctggcggtaatacgtttgttgaagatcagtcacaccatcctgcacttacaatgcgcagaaggagtgagcacagaaagaagtcttgaacttttccgg
gcatataactatactccccgcatagctgaattgttggctatacggtttttagatggccccggtaatctttcatgtcgccaaacttgaagaagattatcggg
gttttcgcttttctggctcctgtaaatccacatcagaaccagttccctgccaccttacggcgtggccagccacaaaattccttaaacgatcagcaatctat
cacgcacaccagaaacaggcaagaatgtgatgtcgctacattttagttagctaaaagcactcttctgctttatcagttcggcaatcatctcagcctgggt
aacgccctcagcttcacacatcacctgcaattgcgcttttaacgcagcaggaatgaacactctgagttctttatgtgtagctcgttttcttgccagggatg
cctgctgacgttctgcaagcgtcagaggattaccttttctgtatgctcgctttgtacctgatgaggaagtcactgcattttctgtctgcgacatctcgcctcc
tcaatactcaaacagggatcgtttcgcagaggatactacagttttttgaaatcagcgacttgagaattgtgacgaagatctgtgttggcgcacaaatca
acggggattactgtcgtttaatgtgatttaaactgtgaaatagtatggttttcagttatggaaacgccgggagcggggaaaacttgctttttcccgtttccg
gggttggacaactgagcaacgcgaaggcgtcagctacgatgttccggggactgctgatcagcccctgttgtattcagccattctccggtcatctgcag
cagttttctggcatcgtggcttctgagcaacggaataccagcctgcctcagaacctcttccagaagaatatcccggcgtctgcgttccggtcgtaaatg
gctggcatcatccagctctactgctgcaacaatactgaacgaacgccgctcaacgataaccacatcaacatgccactgagacaccatcctgaata
actgccaccactggcgtgatcgtggccggatattcccgttcagctggacgatatccgcgacccgtacctgcggacacagataccaccgcttcatatc
cacaattctgaacagtcctttcataaaactgacttccctttctgtcagcagcgtcttctgacgttgccagtattcaccgccggacagtattcgctctgcatc
accggtccgtatacctgccgcatgcaactgcatcagcaaaggagaatgctgatttacacttctgaggataatccggataagtaacgctgcaataatc
agaacaatgagaataaaaataatcagcttcattgtttatctgctcctggcaattaattattgctgaattattaccttacatatttccatatctcttttggtat
cctgtcatataactttccggtgattaattccgctaaccggtggtctgctgctgaattaaatgattcacgttcactgtttaataaactgtctctgtttttatga
ataattttttccagcgtattgactgatgaacatcgccgtaactgatacagccattcctgttttgtttttgccatttttattcccgctgagaacagatgaatga
taagattgccagcgctttatttacggtgtctccatatccaggggggaaccggatcagcatctgaccagagcccacgtttttgttgtctggcttcgttctgaaa
gacaggtagcacgggatctgtgttgtactgctcatatacccaggcagccccggcccggaccataaagtgattaacattcattccatctggtgcataaacctgc
ccgagaatacgaccataacgatctcgctgaaaatatgtgacggtaaccgtttttcctgcaaccagtgatttcatcatgtccgttgaccaccggccataatcct
gtttcttttccggtgcatcaatatttaccagcctgatccgcaccgccttccgtgagtccatgacctcgatggtatcaccgtccagtacacgaacaacccgt
ccatgaatatcttcagacaataccggccatgaaaagataaataataccagtggaatatatttcatcatgttattctccatatcagataaatggcacctgt
atttcaggcatcctgaacattgcagcacacaatgaataacaccttattttgtgaaataaatcacaaaatataaaatcacgacttaatacggaaaaaac
tctgtgctactcagaactgtccggtcatactgcccgaccggatattaaaaaaagcaaatctcatgcattattcagccttctgaatccctggagtaatgaa
ggctgctttttcgttacacccttccccggcagcgccggtggcattgttcagccctttttcgggcaaactgctgccagtctgtccggggtgtaacaccggc
agcccgtcgccatgcttctgccgcttccagaataacggcccggcttccggcactctggtaacgccagacctgaccgttccgagaactgtttctgaatc
cattcattttcctgtcccccctttagttgtcagtattattgtgataataaaaataatcagtttcaccatactctttcccttttttatctgataaacggatact
gaattaataaattcattttccttgccaccgacttattctgtttcttatcaatgcatttcctcacagaagaattaatgctgccgggatattcatacacaccaca
tatactgctgatgtttcagtgattattcccgatgctgaaacggaaaggcgcttttctttcccgtcttcttaagcgctgagcacccgaatctgtgattcaggcg
atgtttttctcctccatatattcacttttttcaccatgtctggcaaagtgaataatgctcatatcagatcctgctcagggaactggctaaatggaccggaccg
gggaagcaattccccggtttttctttgccgcgcacattttttgaattaaatcacacattttattgttcctgcttgtcctgccaggaaaatatgttacaggtat
tgttacaggtattatgtcgtttagtccgattcagcttacagacagggataaccttctggttcaccataataacaatcatccttaattccccttttcagcccct
tcgctccgcaaccgcaacgggggctgtttttatctgagtacagctgcgatacctcatgctttattctcctttttcgcctttttccagattatctttttctccc
tgacttctgtgactccccgacgaaccagttcattccttatcgccactgcccgggttttgttccattcgtcacgcatgtgtgactttaaataattattaaatgt
ttcgacggacacaggcatattaccggctccgctgccggctatctgctgcagctttcctgcccaacggctgacgatatcactttcgggaacagaaggttgtttc
aggcagttacttttgtctgcaccagactcaccggcagcagcaatactttccggtacatcacgacggatgcaggtaataccggcttctttcagcgtcctgataa
caccgtcatatcccttatccgatgaaatgatgtggtatgttttatctttccctgctgcacaacagcttatccgccccagctccagaacaagatgaaaatcgac
attattcctggacacaccactcacctgcctgatagtgattctcgcaccttcaggaaaagagtctgcaggaagaacaacgacgttctgctgtggcccgacaa
aaataatcagttccccatactgctcaagggaaatatttttcagactgccacagttttcataatcaataagggctgtcagggtgtttttatctgtactcgtatt
attttcttccatgtttttaccttttattcataaccgggtgagcaataacacaaaatattttatttccgggaacaccatataaaaagaaagacttccctcgtga
atattcaggcgcatattaaacgacgtcttgttttatgaaatcaatcacaaaacagcacgttattacttaatacagcaataatcctgtgataatgaatgctgac
tggtcatactgccaggccagatataaaaaagaaaaatctcatgcatacttcagccttctgaatccctggagtaatgaaggctgcttttttcgttacaccct
tccccggctgcgccggtggcagtgttcagccctttttcgggcaaactgctgccagtctgtccggggcgcaacaccggcagcccgtcgccatgcttctg
ccgcttccatccagcgtccactgacctccagaataacggcccggcttccggcacgctgccagcgccagacctgaccgttccgtgaactgtttctgaa
tccattcatctttatcactcctctttaacccgtcagtattatggtgttaaataatctactgaattggcttcctttcgggtacggaagttatttccttttaagc
cttatttccctgcctcctctctgatggaggctttttttatttctcatcaagcacggcctgaagttctgcctttatccggttctggggcggattttatccagac
gcgctgccgcatacgcttcctcctcctgagaaatatgctccgtcacatacccttccgtgtcataccggcaggcaccggctttcatggcacacagatagctttc
tgaacgggtgatggccttcagcgccctgcgcagttttttatgcgagagcgggatattccgctgcgccacgtcttccagtaacacgtcccggataccgcaggc
cagaagccggggcgtgtcaccgtcaaacaatcccggccaccagggtttcagggtattcacggcctcatccagcgtgggcaggttcagataaatgg
acagtgcctgtctggcctgcgctttttttgccgccagctctgcttcccgggctgctttctcggccagcttctgctttttcaccttccactttggtggcgtggt
gacattgatgatggttttccggctgcggacaggcgtctccccttccgttttccgcttcagtgtcagtaccggtcgcttctgctctgtcatgctgtgttctgcc
tctgacgaatcatcctgttacggtcaaagataccacacccgtcacagcgccagaacgcccagcacagccagatgacaggcgtagaataccgggaaaa
aatccccgggccagaaacggggtaatgattttcctgcacaggacaccagaccaacggtcagcaccgtcatcacaagaccagccaccgccgctg
catcactgctggcaaggttaagcgccggaataacagccagcaggcaggcgaccagcgccagacgttccgctctgtcttccgcccggtacagccg
gtggctgactgccagcatcagcagcccggcaatgccgtaactggtgccggacaagggcccccacagggccatcagaagaatggccgctgctgt
acgccacccactgcgcgtttcacaccacgtcagcacctgtgccgccactgcaaaggcaaacaggatattcccctcataccagggaaagccggcc
agataatatgcgaactgggcaataatcccccatccccacagccggttaatggccggttgccggatattcgcatgacgggacagattcagcccccac
accagggcaaacagcggaaaggctcctcgcccggccagaaacatccattcctgcttcagctggaatatcagattgatgtggtccagtaccatcagc
accagtgccaccgttttgatgatatcccgctgtccgggtgaccagaccagaaatgactgcaaccaggtatcggtccgtgcagccagtgaatcgttac
gtgtcgtgttcgtgttatccgttgtcatcagtgtttgtcttcctgtatcagtctccgcccagggttttctctttctgcaggtcacgaaccatatcccgctcca
tctgctgcagacgctcctgcagcagattttcccgggcaacctccctgaccgcttcctgctcccgttgtgattcacgcagcacactctccggcagcgcggcttc
ccgttcagttatgtcagcccggtcacgctcctgcccggcaatttccctgacagcctgctctgttttaccatccggcaggtcaggtttgttctctgccattttt
ctgacaatttcatctgcgcggcgttccgtttcacggcggatggcctcttcagcctgccgctgtgccagcacctctgccgtgaccggttcgccatttccggct
cccggctggacgctgctgtccgggatatccccccacacgcgaccaccggtgatggcaccgggattccacggacgaccttcaccggcaattcttac
caccacaccgctgtcaggattatcacgggcaatccggacaccatcctgcatattatcagccagcaggctctctccgttacggctgccctgcagggcc
acaaactgtgcatcttcactgcccttcacccggccttcaccgctgaatccccgcagcccgtttccgtcatccgtggtcagtgggttcagccagataccg
gcggacctgccgttacggtcaaacgccggcagtgccacatacggctgcggatatttacgtcccggagcaataaaccgtgcaggactgtctccccc
ggccagccccgcctgacgaagaacggctcggcctgccgccacgtcccgcagctcccgcgccgtactgaacagccgctgcgcattcatgacctcc
cggtccggtttcggctcaaacacatcgtgggcagttcctttctgtacggcattgttaatggcatccgtccagccctgacggttatcggtgtacacctgcac
atgctgcttcatacgcgacagggccacgtaggctgactcaaagccggccatcagtttccggttaccttccgtgccttcaagcgcgatggcaaaggttt
cactcgcaccctgcgcaccgtgggcggtgatggcataggccaggtcaatatgttgctctgcccgctcctgtccggggcgaatcacccgggtctgctg
tccgtccgacagcgtgacactgtcaccggaaaccgctgtcaccgtccagacgctgttggccacataaccgcgctcccggtcactcttcgtgaagcg
catccggtcaccggtccccacccggatggtgtccggggtgtacagtgtgacgccttcagccaccgcctcccggggcgaaatcagccgcgtgttacc
ttcagcatcctgcagggttatcagcctgtcatccttgctgataccggcaatccggtgatacacattatccaccagggcaagggcatccgggttattctcc
caggtggagagacgacgcagctccccgtcacgtatattcgctgtgttcaggacaggcaccatgacctgctctttccccagctcaccggccttttcccgt
gcatcatgaatcatgctgttcagtacgcgccggtcctcattcaggtgcgtgacaatcagcgtctgctcccgtgcttccggcgtcctgccggtatagtcgc
ggacaatggcttcatacagtgtcatggggacatccgggaaagcctcgcctttcagcatcgccttctgctgcgcttctgccagtttcgcttcctggctgtga
ctgaactccgtcacggagtgctccggtgcccatgcgccctccagacgtggcacctgagacggtttcacactctcaagcccggacagtgccctttcca
cattccggttaatcaggctgtataccgcctcccgcagttccggcgtctgacgcacaatctccttcatgatgaccacatcggcagcactgcgcgtctgct
ggagacggaaaggctgaccgggcgcgatggcctgcagctggtccgtgtcaccgctggccacagcacgaccgccaccggccgcaatcagggc
gtatgcccgtgccatgtcggtattgcccaccattgagctctcatcgagcaggaacagcgtgttgctgaaatccggcgtttctccgctgcgctgctgcag
ctgcgtgtcatgcagaaaggacgccagtgtctgtgcatccacgccggcgctgcgcatctcaccgaccgcacggtgcgtgggccccagccccaca
acccggggacgctcactcgccggcagcatgttcacggctgacatcaccgcccggaactgtgtggtcttacccacaccggcataaccctgtaccac
cgtgaaacggtcggacgtttccagtatcatacgggtggcggcacgctgtcccgacgttaacgtctccatgagttcgccgggtactctctccatcagcg
gcgtgaccgcctccttaccttcgagaatatggcgaagaatacttttttctgcctcatacgacgcacgggaaaccagcaggcctgtgccatagccttttg
ccacatccacatacagtaagtcaccgcgttttatctgcgcattgatttcccctcccagttcagtaaaaccggttccttctgcagcaaacgacttcgcctct
gtcagcaggtccaccatgctgaaggccaggtttttactttccagcacaggaagggccagatgaatggcctgctgtgccggcgtgtgcagcccggctt
tctgcagactgatagcggtttccagcgatgtctcaccggcccgcgcctttatctgctcagaaaccaccgtaaaggagggatggcgggcaagtttttcc
gcagtacgggtttcatccagtgaggaatacagccggacatcacgaccgctgcgggccagaccgttcagggtggcgttgtccattgccatctgtgtga
cggaggcaaacaccgtcgcactgtcactgacggaatgcccgggcgtttccacccagccgttctccagcttcagtgccgtgaacggtgaatcaacc
acaggcagggtggccggttcagcccgccccggcacaacaaccgtcatcgcctcttcactgacggatgccacctgcaggcggtcaccgccggag
acgcggagtccgggaattttccctgtcaccctcagtcgctcgccgtctgccaccggcattttttccggccggaacagcgaccagctgctgtccaggga
ggaaatacgcaccacctgcgtttcgccctgcgcatcccgcagggtcaggctgtgactctgcgctgtcacccggtctatcacatagcggtcatgactgc
gtgtctccgggttccactgctccatcaccatccccggtcggtacatatcccgcagataacggctccggctgtccagccagaccggtgacagtgcagt
catggtcacctcagggtgtccgagcacgccctgcgttttcagctcactgcgaatggcctgtgtcagtatggcctgttcccgtaccccgctgacctgtgcc
acgctctcttctccggctttcacgctggccgcaaaatctccggccagccgggcatagcggacattacggtccggttcactgatgatggtggccggtcg
ctgttctccgccctgccagcgatatgtgttcaccccggcatccttcatggccatcagcgcactgcctgtaccggttcgctgcccgctgtcggttatcagg
acctgtacgttatggcgtgcggcaccatccagcagggttaacgtctctttcagggagagtttttcgccctggtcgacgataacggtactgcccggcgtg
aaggccatgccttccagcagctgacgacgtccggttatcagttcaccggacagccgttcatcctgcttcaggttcatctgcgagcgacggtcagcag
cgataatctgcacctcccgcccctgttcccgggccatcatgaccagttcagccacccgctcacgctgcccggctgcaccgccctgcccggacacaa
tggccagcgacgggcgatcctgtgccagaacactgacggcatcgctgtaaccggccgtccgggggacacttttctccggatgtacggtcacccggt
tctgtttcatgatgtcacgactgagtgcccggactgacagctcatcgagcacatgaatcccggacgtgaacagccccttctcacgatcgagggggat
aagctgctcacggctgatggcctcatcgataccggcgcgtgcccgttcaatcacaccattttccggcggcagtatgccgaccgtcctggccagcacg
tccgtgtacgtgaactgcacttttcgttcacttaatccggcaatcgcctgtgtcaccgcctgctgcacatccggcccgtcctgtgaagctggcccgggcg
cctgcgtgcggatctccgtacgctgatccgccgcgtcacgatatgcccggatgtcgaacccggtttccttcagcgtctgcatccattcagccattctgatt
tccggatcgacgtgctgtttggatttacgcgtatccagtgccgccacatcccgggatttcagcgaggcgtcttccccgacggcctcccggatagcctgt
gagcgtccggaaaaggcctccaccggtacgcccggcatctcccacataccgtgcttacccaccacttcggtttcatagcccagcgcctcaacctgct
ctttcagtttttcccggtagagcctgccaaaggcaatctgattagcgtacacattctcaatgaaccccgttttccccactttgtcactgctcagtgtcttcca
ctcaccgttatgctgcgtgacattagccaccaccgcatgcgtgtgtaactgtggttcctgatcgcgactggtgtcgtggttaaacagtgccatcaccag
attaccggtcagcaccgtttctgactgtccgtccgtcatcacccgtgtggaggccagcgcctccacctgacgaacagcaaagtccacggcctggttat
gtgcatcaatcaggcgcttatcgccacctaacatggccatcatggagacacttttgggggcggagaaggtcaggtcgtagccgggacgatgcttatt
actgccatcctgcatgcggcttagatccgctccgtccggcagccttccctccagaagacgggtaaaaacatccttatcgacgctgccctgcagccca
agctgttcagcccccttgccggcccagcgttctcccatgctgcccagtacatagtaattatccttgtcggtgtaatagttaccggcacttccggccgatct
gacctgcgcgatactcatcatcagaaatcatctcccggctcaacatcctccccacgctcccggtgcacattaatgttcacctcttcacgacgctgcatct
gctgctggatgtccggatgattttcctgttgccatgcctcataagcggccatatccaccacttcaccggattcactgatcccgggggcagttgctgttcc
atctcttcttccggtttcattttcagctcctgctcgatcccccctgccggaatattcacacctgaatctgacttcttatcgttgatggcaggagacaccggct
gttgcggctgttgcggctgttgcggctgttgcggctgttgcggctgttcagcctgagtcacgtcttcttcggaaacaacttccggcacatccggttcgaag
aggctggccatctgacgaccttctgcttcccttgcggcaagtacggcactcagacggttctccatttccgggttgatgtcacgcggaataaactcaggg
gcaaccttcggacgtgcctgatatttcagtgagagttttactgccggatacggtccgggcagggtgacataacaggtcagatccggcagagactga
atgtcggaataactgaccagcgtctggcgctccatatccttaccggtcgataccccgtcacgcacggggtcagcaccgtaggaatactgcagactg
gctttcaggtgctctttctcaccaatctcaccggcagcgaactctgcaatcttatggctgggagaacggaaaaaggcacgggtgttcatgacgtcaaa
cagcgtggcagccgctttctcaccgtagatatcttccagctgggcataggactggataccaaacacataacagccaccgaacttacgggcttccgg
caggatctccaccaggtccggcagtttgtgtaacgtgggtaactcgtcacagaaaaaccacacacgacggttacgattttctcccattgccagcaga
ccacgaatggcaatggacagccacatggagatcaccggtttcagggaggcatgggtgtcggcattcgacgaaataaacagccagccgtttttctg
atcttcccggacaccccgcatccagtcacggatggtgaagggctcaccgttatgctcaatcccctgcaggtaacggatagctttcacgtagttggtca
gcacagcacggatggaaattgccgttttctcaattttctcttccaccaggttggccgccggtgaattacgcaggaaggtacgcagtttttcaattttgatg
gaaagcagtgtgtccaccagtttgctgtagctgcggttggggtcattacgcatcaggtacgccgcttccgcaaaaatggtacgtcctgaaccctgcca
gaacgggtcttcttttgttcccatagggatcagagtatttgcggtattatcaaaatccggctgtgtcaggcactccttccacaaatcccatgcggcacag
cgggcatccagcggattcaggattttatcgatggagggatcatagtaacttttaacaaattcccctgaacggtcataaatcaccaccatatcaccgcg
ctgacgggcgtagttggccagacgacggataacctccgacttaccggccccgaccgtgccgtgcagacagaagttctggatttcggaatcccggat
aatcggcaggtcgccaatccggatatcggagtccttgccgtcttttttcagcatccgggcaacgtctttcggattgtctgtcagctgacgaccacctgtga
cttcattttcgctctgttgtttaccctgacgccccagaatccaggagacaacaaagaaggtgatcaggcaaataaccagggccacgacagacgcc
agaacgaatgcagaccatagctgctcaccgcaccagatcatgtatttatcatgcagtacctgagccgcgttcatccggaaggttttgccgtaatactg
gatctcatatactggctgggacttgattaaatcccgcatgccttccagcgtggtacaccaccagtaaatacagccgttcacaaacgtctgccagcttatt
tttacccataaaaccagaccaacgagtatccagaaaaaaataaacaggcagtaaagcatgatattggcgatctggctgaacatgcggatacgcat
ggacgcaatctgaccgccctgggtcatatcctttgcgttaaaactcatgatgatattccgaagaaaaaaagtcagtcggactgattattaatgatgaaa
agattaataaccgttaaattccggcttcgccggcaacacttcacggtcgcacaaagaatttgcacaaccgctcagtgttttcatatttatttcggaaacg
ccggggagaaataaaatttattttatttctttccggggttggacaacgagcaaagcgaggcgtcagtcaggaggccggtcaggccagcctccggaa
gataatcagagaatatttgcgattgatttggccagctggtcttcgagaacaggcttcgcctcttcaaatttcaggttaaccttgttcgcatttgagaccaca
cgggtctggtatttatgctggttacctgtttcagtactggtctgaattttcgcaccggatgtgccctgacgcagggcggcaacattatccgttgtcaccgttg
ccttagtacgctctgcaatctgcacatccgtgatcatggtatagttcacatcttccaccatcgcatctgcagccatacccaccagaccggcagcaagg
cccacaccgagtgtggcaccggcagaatttgagttatatccggtaataccggcacctaacgctgcaccaacagctgcgccttcataaccacggttc
agccatccctgagactcccgcagatccatcttatcggccttcagcacatttgcctgaatccagtagtaggctttatccggagaagtcaccacctgatat
ccttttgctttcacagcatcagcaattttgccctgcagcccactcatgtctttatcagacgtgtttttgatctgcagaaataccgtgcgttcgctgtcgggtt
taagccagatggtttcactcatctgagtcttcacctcaaggttacgcttcttgattgctgtgctcatcgcaccacaccctgaaagggccagagtggaactg
accagtgcaaccatcattaatttttttgttttcatcaaattacctttattacaaaccacagttttcttatcgcctgtcatcatagtcctgccaacaagtccca
aaagtatttcctgctgaatcaaatcctccatgcatagctaatcctgttgccggattagtacgataaaaatccacataatcaaaattcacatcatcaaaatcc
agatgcaaatcttcattagtttgttcatttacctgagattgcatccattctcttacacttacacaaccactaattcgccttaattcgtatagtgtaccaatga
taaaaaccatcaggcttaataacggagagagtaaggaagataatggcatcagtatataccaaacgcctttactgaatttaaactcatccgacacatgc
cgtttttttcgtatttgagaaaacaaccatataaacaatccctgccatagtacaataaagcacaggataaaaactgtttcgtcactcccccaatcagcc
agataacaggcaatgaactgtatcaccgtcaccagagcaatatgagctaaatttttcattatcatctccccatacgatattattgcttgtgcttatcctgat
actcctttgctcttttcatcagttcctgtggactgtcagcaccaggcattctttcctgacccgatttttcttcattatatttattattctgagataatgcctc
tgttttatggtgattttgtagttcagaatactgtcttcctacaatattttcttcgccacgtatgctgctctgagtatctccgatattgccacgatattctgtg
accatattatcaacctgatgttttacatcattacggatattactgtcctgcgttctttgatcaataatggcctgatgcccctgatggtcagcaataatatcct
ggctgccaccacccgaaggtacgctctccattcctttaccaatatccccacgggattcacgccaggcattatcaacaccaggctgcacctgttcctgaacaaa
agaccacgccatagcccgacggcgttctgcaatctccggcgaactggtatttgtcagaatagcttctgcatcctgcggtgcgcgtttcatcacatactgtgca
aactgttgcgacaggtcttcactcatctgcccgctcatgctttcagtacgggaggccatttccgcatattcatggctgcgggtcatattcgtcgtgtactggt
cgtaactctgtttcgctgagttcagggctgcagacagctggtccacacgggaatcggcattattgtccgtatggctgccggattcactgaccttgcgactggt
aaagtaatcgctggcctctttaaagtcttgtgtagccctggcatcgatatcatggcgagcatcatggctggcccgtgaaccactgctggcctgatgcgc
atcctcatcactccagtcaatacttgcccgaccaccacctctaacacctgcgccaccaccaacaccaagaattttaggtttaaccccccattcagcat
gaacatcgccatacatccccgcagaacctctcgtacttcttgatgccagttctcttgttgcctgctcattactgatattatgcgccttcgcatagctttccac
tgcactgcgcatacggctggccatcatggagtcctgtgcactcatggtgctgtcagcgccaccggtgacagaatcactgctcccccggttagaacca
aactgactgagcgagttccaggcactggcaatactgctgctgaacccatgcagggcactttctgctctgttcgatgcttcccgggccatttcctgttgtgc
cgctgcaatctgacgcgttgcattgataccgaccggtaaacgggacatcgctccgcttgcatccatcaccatattaccgtcacgggtctgtgtggcagt
tgcgccactgccggtctggtacatcatctgaccaaacgacgtggtgctgttggtgctccagctgaagccgttcacgttttccgtctgcatgttgccgtagg
agtaattaccgtcaaccacactgccagctgcactggccgtcggactgatagaggaagaggcgaagtggctgtacacgctggaaaaacctgcgcc
aaggccttttaccatcatccatgacaacggggggatcatggctgaaagatacccggcagtagaggccaggtcagagtatttcagctgtatctgaga
gagttcagacagcacgaccggcgcaccattcatcttcgcatagaatgtcatggcactgttcagaatggcatacaacaacggccaggtctgcagcc
acatcagggcaaacacatagcctttcaggacggacagtgtcagtttgttgaagacagctgccagtaccagaagcgggaaaataccaatggcaatt
cccgtcaggatagtctgtaccattggcaggtttcgcattgtcacatgaccgatagagacatgcgccagacgttgcttctccatcgatgacgtggtggcc
agattcaccaggctggcggtgtcaccattacgggcggcattactggtgataccctctttcagggcattcatggtgacgttctggcgcataatctggctgg
cagactgactggagccgtagaaataactgtaactgtcactgaccagttgtctgaataacaggtccgggtccggtctgccgccaaatatctgctgcac
ataataatgccaggtcttgcctcccgtctttgtatcgagattcagcctgtctttcagcgtgaccgacgcatccttacaggttatgaaattattattgttgtca
taaacgccccgtagcgggctgggtcgggaaaaaatcagcgtgtaggggtcagcggaggccatcagatcttccagcgtgtatttatgattcaggtaaat
atcacccagcacacagttctggacgtagtcctggaacagattgatgatttccggattccgggacaggaaatcggtgcttttcacaatcagattcgccc
cgaacagcatgcctgtcttgctgtaggtgacgctgtccggttgggtgaatatcatctcgtagctgaccaccatcgcatgcccgatacgggtcgtaaga
gaaagtggcatcgccagaccgaccggaacattatccacccgatggactttgaccaggtcactgttatcaataatctgcaccgatgtgcggacattaa
ccagcaggctgataagcacaaacacggccacccagcccagcaaatccatcacgttatgccgctgtacccacataaaagccacagcaacaaca
gacagggtgagcgcgattttttcaattgaatcccaggtccgggtattcataaatgctgcaatggcattcaggttgttacgcaaccactcaccaccggca
atcacataaacttcattcacagtgaattccctccgaagtgatagttgttctggtaacggctgagcatacgggcggaaagctgctgacgcatgtagctc
atctgacgttcgacaaccagcagcgcatcctgctggacctgtacctgactctggaaaatggcaatctgttgctgtgcctgattcagattctccagaaca
ttgtccatcacagcctgcggatagttcccggtggcaatcatggcccgggcctgctgaagcagttcctgaatgtactggagcatgatgtcgtagccgat
atagtccgtcagctggtaaacgacgctgttggacacaccgagcatctgcgggtcaatcagatatttaaaaacgggaatggtggtgctggaaataaa
cccctgttcccgggcgtcaagcggagtgtcactcactgctttattctgaatactggtgagcagttttgtaatctgtgacttcagggctttatcccgggcgat
ggtcacatttgcatccgccaccaccttcagacacttgtccgaatcgttgcagtggtaaatttttgccgttcccccctccataatcgcccggaggatatcgc
ggtcagtggtacgcgccggcaggggggtaatctcgctgtcttccccaaaaatcagcgtaccggtgagcgtcatgataaattctttcaactctttgttgcc
gtcaaacattctgttttttttcagcgcctcccatatgatattctggtttttcagcacccgctccttgtccttgtcgctggctttgtcacgaacagatgaggac
tgccctccgaccgtgcacccctgacgagatgcggcccagtcggcgaagatattgctttcactgccaatgtcctgacaaactttctgctgagattcctgcgtc
tgagggaacagcccccccacaagcccctgtgccacctgacaggaactgaggttcatactgttaatgtcacttgccattttctgcaggaagtcttttgcg
gtttttatttccggcaccgttgtctgcagggcaaggtcaaaaaagtaaccggcagcattgctcataatctgcttaacaaaacgctgcaactgatcgcca
ttaataaaactgaacgagccgagataggcgtcgatgccgccacatccggcattgatatccggcagcgtcatggaaatcagctggatatttttgacct
gggtgcgggcatacagggagccaccataggcatacccggcggcctgcccctgccagacgcccgcctgggtgacattggacgcaaatcccagct
tattaaagaactgattcatgtcactgttcacatccgctgacgctgcgggtgtgaccgtcagtaaaacggcacaaagaaccagaagaggtttaatgcg
aggcatcgttattcccctccctgaaactgttctcccggcgcatacggcgtttcagccggaatatcctgatccccccgtttattaattcatttaaagggttcct
gacgtatacgataagaaatccacctgcaaacgcccataacgtatacggaatgacatacatcaggatgttccacatataacgaaaattatttaattcc
gggtcagctttccacgaatcaatacaggcatcaaaaatactgtgcataatcacccataaaaaagttccggcagcaacgaacacaaaaaacagat
caaaatatatatcagaaaccctgcgttcataatcataattccgtttagtttttctgttttttagttccgcatcatttcttttttctttcattataagtttcct
ctgatatttctgatgattcattcagacaggttatgcggctgtaatatgacaccgaaatccatcccacaattgcccccacaaaaaacacaacagaaacagaaaa
ggtaagacacagcgaaacgacaatttccacagggctggcgggataaaaaagcccctgacggataaaggggatattcaggctgacgattattcccgt
cgcgatgccggatattccggcaatgagtaacaccacctgcttatttcgcacctttttcctctccgtatatctgcagaacggtatccatccgcgccataaa
actggcggcatccgttgccccctgcaaaagcggtaatgcctcaagtgtgttgacgttgaccaggaaggtggtcggtgtggccaccgggatattcggg
aagaaggtctgcatcacgtccggtggcaccggtaatgcttccggaaaggcagtatccccctgaccatccagggtgtagggaaaaacggaaaagc
cgtactgctgtgccagctgcttcagtaccgggtcaaactggtgacagtaagggcaacgcccctgcataaacagcaccaccttccagtcggccaggt
taacctgcctgccgttgctgaggcggaaccagcggggagccggcttttctgttctggctgatgtgtctgctgccggttgtgcaggctgcacggccatac
cctgcggattccagagtcgttcaatttcatcccgggtggaggcctgcacacctgtggccatcatcaacaaaatgaccggcagtaatttagtgagaga
catgtccgccctcctgctgcccttccatcatggcctgttgcctggcttccagtgcctcacgctcttccaggctgacacgtgcaatccatgcatccagcac
ctggtatgcgccgaacagcacaaggatggccgcaatcaactccgccagaaaaaaagccatccagggtttgctgtaaacaagacgggcgacgat
atgaaaccagacacccagggaaaatacccacatacccgtaatatcaagccggggtaaagagaatctgcatttacttatcatgttaaactccggttat
tatttacatgagaatattcatgtctgtaatattcgcgtttgtgtgatattaatcagatcaacctcattattttgtatggtttttattaccgttttatatgact
cagcctgtcataagccccttataaaaaaccacatcactccggaccatgctgatgacaacataatacagataaatattcttctgaacctgtcgaagctcgtaac
gttcggtaatgagccaatagccattggaacaaccattgctgcgccctgattcataattgcaccacacagaaaccagagactggcggtaagcacatata
aacacgcgatattaataacaaggaaaaccagtgaactcaggagaaacattttcctgtcattacgctcagaaaagaaataaccggcaataatgaca
aatattaacagaacaacacctgtaaacattttcaaataatcttcactcataaatcaaatccgcgattaaaattttggtttaaaatcttcagaaacgttcag
gaattgttttgccaggtcgtcctgcgaaataaagccgtatgataacgggcgaacactgccctgtttcgggtcaaccagcatcatggccgggaaatatt
tcacgccgaggcgctgcgcctgcccctggtcagtccgggaatccggcaacagcggattaatcacgccatccacggaaacgggaataacactca
gaccatacgtatcccggaagccattaatgacctgcgccagctgcccgtcgatggggtcctgcccccggtaaaaaaacatgatgccgtagtgttcag
ccagtttcgcaatggcctgtcgctgctgcgcctggtctgccgccagctggttccgtaccgtgccgttgtaatggctgtactgcaggttatagtccagttcg
ggatgtgccagcatggcctttttagcactcatggtgaaaagcccggcctgctgagtccagtaattctgcagccggaaatatttcacaaaattatccacg
ccgggatacagaatggcttcgtacagcgcccgctttgttgccgtctgcagcgcggccagtttctgcataatatccggctcctgacggggggctgcagg
tacaggtttattttctttttcctttggacttattttctcgttataccactgccagcctgcatctttccctgacgccggaaaaataaagcagcagagtaacagt
ggcagtaatgctttattcatcttcattcctttcatcattttttctttcatcagctttctgcaggccagtcagggcagaaaaacaaaaatacagaataaacagg
agcacaccggagtaaaggataacgaaagcagcggcaagcatcgcgtctgccgtggtttcttttaccgtatcccacgaccagataattaccggaga
aatgacaatcgctgtaatcaaaagccggataagaaaatcaacaagacgattagaagtgaatatgactttcatcatttcccccggttattttccatttctta
ctgtcctgcctgtttcagctggtcagcaatctgctctttcactttctgcgtcagaacgccactgtccgggattttctggttattcatcaggtcttcgtagaag
ttagtgaaatccagtctgtcgaactgaattttctgcagctcatcaaccgtaatcccccgacagtcaggatgcttcgcactgccaaaactgatacgcaact
gcccgttacgcccctgctgctggactatctgcgccagcttcgaatcaaactggcagtagctgcgctttttctgcagacagacacccagcactttccggg
agcagaactcgcccacactcaccgtcagtttgttgtcctttgcctttcccagcgccttttcttcactgttgcacttcgccagccccacatcctgaccccagc
cgccatccttacagcagttactgaagccggctgcggccttacggcagaatttcgcctcacccgtgaaagccctgacatccacaccattcagggcgg
caacatccttacctgcggcggcaagcgcagccagctgtgatactgcttcaccaaaatcactgctttttccgctctgtgccttatcgcattcaccgtcaag
gcagaagacatcaccaccacagaccatcacttttcctgatgtttttgactcgcaggagtacgtggcgtactcatgcagacaggctccctcatcggaat
aaaacgcgcactgacgtgaggccagcgtacaggccggattatcgacatatttctgacaggtgccgttatctgcggactgagtgacatatttatcccgg
tatgcccagcatgcctgtgtgatgttccagggtttaccttccatcactccggttttcgtaccacccggctctgaacactcagtgcctgtcagcacgccatc
cgctttattaaacggacaactctccacccactccacccggggaacccattctttatcacggaccttcatcctgagtttcagcgtaaaggtggaagcac
cactgacaagcgattcatagactttcctgtcaccattcccgtgcgggaggcaattaccgttagcagtacagccactaccggtcagaacctgcccctgt
gtcacctgcaaacctgtcgccccggacagggggtaagtatccgactgcccgtttgccaggccgacattaaaggttgtattcatgaaggtataacggg
agttcaggaagaaaaaagcggcgtatacagacaatgatgcactttccactgtaccggttaccggagccgtgacactgaacaccagtttcagaccgt
tcatactgaaccggaactgtgagtgcggtatcgtcacctcacgatattcatccgtgtatttccagtccccggtgatactggcagtccgtgtgcagtactcc
tccaccatcgtgtcccgctcacaggtatagtttgtgaactcactgcggtttatctgctgcgcactgcactgctgcccggtgtttcccacaatgtgtcagcc
cggttcaccacatccctgccggtctggataaacggtgcatccggtgaaagaatgtctttcggcttgttcatgaatgactctgtgatagtttttccggtttcac
cggtcgcccattccgtggtgccgtcatttttcaggccgccatccccgccggcagtcacgccaccgtaatattttgtctcgtccggattcgcgttatagccg
gggatactctcctgtggcttaaagccctgaatactgcctgtaccctgcccctgaatctgacgggcaaaatcagaacccgcccggtaatcactgttgct
gtcagcctgtgccacactgactgccagccccagcagcaatggcagaagacgtttcatacctcactctccctctgatattgctcaaaccagaacacga
ctggtttgttcaccacctgaatcagcccgaaccgttctgcggtcctgaaattcacgctgcttctgcgcccccgttcaacctgcagggacacctgcgttct
gaacatctccagcgaatatgttgttttcagcaggttgcacggcgcacaggccgggacgaggttttccggacagtcggcctccggtctgaaaacatca
ccggtggatttcagcctgaaaatgcgcttttctgcagccttcatgcactgctctgacttgcggagcaccgcctgaacatggtcggcatgccatcctttctc
cggcagttcacagccgcagtacgcacaacgaccgccgaatttcatacgcagtttttcccgctgtttctttgtcagcgccatcaatgcctccttacggga
acagatttacggattcagtaagcggaccagcgtcctgaagtctgcacgcagtgagcgacgacagtaccggtgataccagtgatgagggccttcag
gctgaagccggaaacagagctgaaggtcactgttaaaagaagccccgacggaccagccccacagcaaccggggcatcatggactccgcatcc
tgccgcagtcgctgctcaagaagagccgccttttttaccggacagacaaaggcaatataaggcggcagtttcgtaacagggaaaccatgcccctg
acaggacgcatatgtacaaaatccgtgaacgttcatcgcggccaccagtgcacggatgtccgggtcaatccggtaatagcctgcaggtaaactcat
tgcgacttatccccggtgccgtccagcagtccctgagccacctgccggcagtctcctgcggtggccactttttccagcgcctgcctgacgcgcagattt
ccccggatgatgtcgtgcccctggctgcagaacaccaccagggccggaacgctgcgaatgccgtactgtgaaaacagcgtcgggtcgatctgaa
caccatcagttgcgccgtctttcaccagggacaacacggcgtcagccgtggccttcaggtcgttattcaccatgccccgcagcgtggcgggaatacc
gtagtgccgggtttcgcccagcattcgtttcagcccctcttcgggaatggaaaacgacacaaaatacagggcaccctgccggggttttccgtcctgcg
aagcctgctgtttacggaccagttcatcgagaaactggttatcggaatgctgaaggggattcgccctgacttgctgctctgcccatgcctgcaactgtgt
gtccggcttttcacgtaactgctgactcagtgtttcctgctgcttcaggaactggcggttttcagttgtgttcatattttctgaagcatgaacagagataaac
agtcctgctgtcagggcgaataaaatgtatatttttttgttcatataattcttctgcaaacaggcagtaattaatcactacaaccaatatctgatatcaacaa
ccagcttctcacggtatttcagggattattaatcccgggattacgcgcccggtcgcgttgtggtaaaatatttcagcctgatatatcgattgcaagagatg
acaggctaaaaacataccaacaaggagttacaatgaataataacacatcagaagaccgccctgatttttcattcagttacatggacaatcagagag
cagatgaaataaaggctttaaaaatattattattcagcatcatcagaaatatgcctcatgatacagccatcagtattattaataacatcgtaaatactgat
aataatgaggcgctcaatagattatgcctggagataactgacgccatgttaaacacaacgagaatcgttgattagcttctttccccatttatctttttctg
agactggctgcaattaattgccagtctctgtattgctctgctgttattacctttttctccatactgaaagtgtcacggggtacagggagagatacatcctgat
gcaaggggagctgcaggccacattgtttctgttctttttccatcatttatccttttcttgtcagtgttataaaaatacacagttacgttttcgcgtagttgtc
tgcttaaattttcctgctgcttcaggaattgtcagttttcaggagtgttcacattgtctgaggcatgaacagtgatatgcatccctgcagccagggcaaaaag
tacaaatattttccggctcatatcatctcccgacacagtgatataatttaattaccggcgcacttttctttcagtctctcagacaatgcatataattcatcta
tttcttcctgagttcagcccattctctgaccacatcaaaagcactcaccctttccagcagggagaaaaaacgctcctcactgataaaaattttttttctttat
tacttatctttcgtaaaaatgataaaaaactcataacacacttttatttatattttacatatgcacacacggttatatttatgagaaccgacagactcatttc
ctgcgcttaacagacataaacttatctgacgaaagagacaagacaacaacgcttaccactgtcagaataagaaccgtcttcagatacagtttgacaagc
agtattttttccagtgtatctgcatcctgccaggcgttaacaatgtctgagataatatgttcagggcaaagataaaaccacaccacagggcatatcacc
atagcaaaaggaaacagttttaatacattgcctgtcaaacgaatgaacactcgggatatactcatatacatatgaatgtatttactgcttaatttcttttcc
atttaaaatattcctctttttgtttaagttacagaaatacacagttacgtttacgccacatcaggtagccgaagttttttttgtgttgggcggattttttccc
gtctcccagcgcatcacactgcgcccgaacggatggcactgcccgctgtccggatacatattcaccatctgataacgccagcgttctttgggcaggattgg
ggacggatattcattacagacggcgttatttttcccgatggtttccataatcatgccctgacggtgtagtttgaacgccatgcgttcactgaccagcaggg
aggactgcaacggactggactcattactcacccagccattgaacgggtacatacttccctgcgaaccggcacaccagaacagaacatcgagagg
catattaaaggcgctggcaatggcatctgctgcacaggccccctgtgctatcggattggcaaagatgacagcttccggattgagaatggtggtcagg
ctgctgtccgtccaggtggggtcaatttcagaaagataagcgatatccaggtcaccaccttccagacagcccagcgatgtgatgatgttcagccagt
acgtcagcgggtatttgtaccagtgaacgtgatagaacgccccgttcacctgcttctcatcctttttcgccgttccctgtgccgttttaccaaaagccggc
aggctgaagcccaggttaaccatgcatcccggtgaccgggtgacgtccgtcagcgccatcggctcccagtaaccaatggccagcccgatacgcct
gaacagcggcggcggtgccggacaaatctgaatgggcatcgacgggttcgccgtgtcggggaccttaccctgactgactttgatactgcccagcg
agagcgggaaaatacagctccagcagatatccgtgatcgggtttacaaaacgcccctcacaggcagaatccgcagatgccgcagggacatgac
cggcgagaaggaataacatcagcagccacaggcttcgcttcattttctgccctcctctgccggaataaattccaccttcaggaagcgatcgccggga
acggcgctcacccgtgccgggacctgatcaatgccgaggcgctggcagagcacaccgttctggtcaaagtaaatacggctgtccagcgctttctgc
atctccgggatactgccctgcacgaggataattttgctctccagtgtgggcggcgtctggcgtttcatccaggccacctgcgccggatcatcgccattg
atgaaatacagcgtctggttaaaaggaacatactgcagcggattcatcacttcaccctgacgggcaaacacccgcccttcgttatcccggatatccg
cagccagtctgacggacgggtcaaacaaccggctgctgtatttctccgtgcgtccgataccgggcacggcagggggccgcagactgttacggatc
acccgctccttaaaggcatccattttccggcccatctcaccggactgctccagtgccgtcaggcgctgcatgatcaccgtcagcatgtccggctccttt
accggccacagatcgccccaggtgccaagatcggcggcggccacactctgcccccatatcagcagggcaatcagcccccggcatctcatggttc
cgccctcattcgccgggcaatatcctgctggatttcccgggtgatatccggtgccccctgtaccacggcaggcgacaccagaatgaccgcgtggtgt
ttctgctgccatgcctgcaggctggcttccagtgccgtgttaaaacgcgctgaaagggcttttgactgtgcttctgacagttgtttctgactggcgctgtca
aaaaacgcatccacggtctgtttcatgttgaaggcggcagtcaccggtgcgttcagtctgacaatgccataactgatggctgcgttcagtaacaccatt
gccaggcatcccggcacggtccaccaccagtggctgcgtcggggggctgtaacgtcagtcgttttttgcgttgtggtcatgcaacactcctgtatttctc
atgttcttccagccaggcctccagcgaagccatctccggccccgatttcttccacgccagctgccacaccgcctcatgaatactcataccctctctgcg
tttctgctgcacaaactcaaaatccgggccgtcagagctgtacatggcacggcttaacgggtccacaaacagacggtgccaggaggaatggttttc
cacctgcagcaggaaggaactgaaccactggtctttggccgcaccaaacttaccaatcatgtcgcgctgcagtggctggaactgatccgggaata
actggttatatttcgcgaactccttcgcactctgtttgaggataattttgtaggaggagttaccccatgccgcacgggctgcgctggatgccttgtcagagt
caaagtcgacgatgttctgtgtgatggtgatataggcaccggtatgacggcgacaggtgcggtaacctttttgaataaactcaccaactttacgatttttg
aagtccagcagacgccagccttcatcaatgacgttcagtttcttgagggtacgcggcgtgcggtacatccggttctcgatgtaaatgataaggctgaa
catcaccgcaaccagcagtgacggacggtcttccagtccgcccagctccagcaccaccatttttgcgtcatcccgcagggacggctcatcagagtt
aaaataccggccgtaagtgccgttggcagtgtactggtcaagcagcacaatcatttcgtccagacggctgcggatggtcggtgactcgacatactgg
tcgttatcacgtgcgtttttcaggaaatccaccacatcatcaatgcgcgcctgcttctttttagccagccaggaggctctgaccgcctgcagcaacaga
ccttcatgcacttcatccaggttaccgttggggctggccatcaccgacaactggtcacggacacgctccgctgactggtcaatatcagtgatgttcgca
aacgggttaaagcgcagggtttcaccgtccagatatacgccccccatgttctcacacaaagacttgtagccatcccccatgtcgaacaccacggca
aagcccccggagtccagcacgctgcggataagtggctgtatcaacccggttttaccggccccggatgttccacagaccgccatgttgtagttggtgtt
attcatccccttaaagaagatatcgataaacgccagctggttacggtaggtgggtgccagcaggcctgccggtgtcagggggttatccgccactaac
ggcatcaggttggccacattaaagctctctgctcgctgaaccacaccggcctctttcagctgtttaaacagccctttcccggccataaagggcaggca
ggtcaggaaattgcgcatgtggttaaagcgcggtgaaatcagctcaaaaccgtttttacgaaagctgttcagaatgtcctgctccacttccagcgccgt
ttcattgttgtccttgcagaatgctgtgatgttaaggaagtaggacacgacagaagactgaccggagcccagccgctggcgcagttccccccactcc
ttagcttctttctccacggagggaaaccatttggcataggaggtcttcgactttttctccaggtccatgtacttcaggttggcttcactgtgggttttcacct
gatcttccaccaccagtgtcagcgtcaggatgaacgggcaggaaatggacatttccgggttcagcagattgctgtagttatcggccatgttccacaggaa
ggcgatttccgggttacgggccagatggaaattcaggatacgggctgtgctgttcctgccgttctcacgcagccccagcgtcaggtaatccgcccgg
accttcagatcaaaactgtcctccacacactgataattcagatcagaatacgggtccaactgacgtcttttcgggtacagagagtccggattatggtta
atcatctccccgacaatctcgatgaaagcctgtgcatccactgtctgtgtggtgatttttgcgccgtgaaaagaggcacggatgattttcaccagattttc
catttccagaatgtcggcccggctttttttcttcgagggagaacagtacgagataaacacccggtaatgacgcagggtcaggggcagattcatcccct
ccggcagcgggaactgtgtcgctgccgctttcatataataggcacgggtaatggcgttaaaccgttcagcctgttcacctgaccaggagaactcacg
cagcccgtattcaatcctgtcaccaaccagctgactggacatcagatggatacacaacggaataccgcgcggcagtttggtgcgcagcatatgatc
gagcgcctccacaatggactcattcgcaccattgatgggaatggcttccagcataaagcccatcgtggtgtcattcatgaacaggccggactcctgg
ttgtaatcacggtacggcagcagacggctgaactgcgggaagctcatttcgcccagaacttcattggcctttgcggattcgtcaggcagtttcagtgct
gtgacgagggagttaaccgcctgagtgacggcctcaagtgggttattcacatgaattccttagttgaaaaataaagcgagaaatgcggtagccatg
aagggaatacctgtaattaacgcgccatttcccggcgcatatacaaagatttcggttttctgaatatcaactcatgttcatccggaattaaatcaatatgtt
ctgcgactgacttatgcataatgttttctctgcctttcctgatacgcctgacattcaacacataacgtcacaccgggaaatattttccgccgggcttccgga
ataggattaccgcatgcttcacagagataaacaggaataccatgagcatttattttctggcgtatccggtttattcctgtcatggtcagttgttctgttactg
aatatgcttcatcgacttcatcactcacggaatattcctcccgacatgttctgacacgctaccgcccatcccttttcacgcacagggcactcgttttacagg
cagattttgaatacagaaagcccgccggcattgccggctgatatacaacttcagggccagttaattaatacgtggttttccccacgcagacggtttaat
aacaaaaaacacactggatggctgatgataaacatcctgattatcaatataaggcgctatccataatgcagccgtctgctcacccgttcttaggggc
ggggaggtgttaccggcgcaactgaactgaccggaacaatcgttttcacttcccgggcagccgtaaacaacggacgtggtgccagcagtttctgct
ccggaagtgccgtcaccgcaggtctgctgccggacggagtggtcgcggtgatggtctgcaccggcattgtccggaaattcccttcagccaggcgcg
gcagtgatgccgcaaccggctttgcttctgaggactgctccagttttttggccttctcattggcctgctccatcgtcatacaggtatcggatgtggtggcgtt
acactcaaattccgtactggttccggcacagccggaaagtaataaggtccccagcagaggaataaataaagaaatctgtttcattgtccctctcctttc
tcatcgcagttcacaaaatcattcttcaaatccctgaccgtacaggtttttacaatgtcaggattttcaaaaaccgtattccgtgatgatttataaacatatg
ccacaatgcgtttcccccccgcatcaggttctggtgcactgacttcacataacggaggtgttattccggattcatacagcaccggataatttatttttttga
cgctgccatcagaaaccagcttattcatcgaagggttccttatatttctgaataattctgtcgattagttctttcaccggaacagattttccgccaccggatt
gtccggcaaccacaatatgccccggagaagaaaataagctcatcgacattcccttcttcatcacaccgacagcagccatattaaaattgtgcggca
atgattcagaactgaattccttcggtgcaggaagtacgattcttgtggtgcagttactgagaacagcatgcataaaatcatctgatacgcttttatccggt
acagagcgggcagtaataacattaatattacgcatgttcatttttatctccgttgtttctgtttgttctgtcatcaggaaaataaacagggcatttcctgatt
ttttccggcagggaagcgtcttccgtataaacttccgtcagacgttcatctgcggttaccccccggatacatttcatttttccgggcagcaccggatatttcc
cggagataactgccagtgagacatacatcaatgccagtgttgcccataatgcggtaaccagttgcatcagtgaccggtcagtctccctgacataatctg
cagcatcagcagcacggtaacggcagattttctcttctgctcctggaagatcatgcagagaaggcaaacactctcttacaggaaactgcccggcag
gggctgtttgtcttacgaagtcaatttcctgaaccagtaactgccctggcgtggtgttgtccttccataaaacaaacagagccatcagggccattgtggc
cagtggcagaatgacggtgtacttcagacaccagaacagtcctcgcagaacccgggcgacaacataccgggcggcatggcacgcctgacggg
acgacatattatttgccatcgttgccccctccctggctgccggacataaactgctgcggattcaggttgcccagctgcttcagcatctggtcagtattaaa
gccgttaagatgactttcagccgatggcggaacaggaaccggactttcctgattctcttcttccgctctgctctgcgtgcgttccagcgccatctcttccac
ggttttcagctggaagccgtcctggaacaccacggtcacttcgttgcccgcaccaatcggtatcaccgggtgatactgttcggcacgtttgatgtagta
gtcactgagcgtctgtgcggctttcgatgcgccgccaccgatacccattttcaggacatcaccagccccgtaagcggctgtggcacccagcccgac
agccggctgggaggcgcgctccattccctgaccgataccgtcaacaaatcccgcgccccatgcccagccgaggattttgccgttacgcatcacca
cttcgcccttgataccgtttttaccccggaagctgacatgccccttaatcggcatatcaatggttttgccgtccttcaggcagctgatattgcgggtgcgta
caatggcacgttcactggacacatccccccaggcttccagccccacaaaacatcccgttgcgtcatacgttttgctgttcggcatttccaccagaccg
gtgatacgcagctgcatcggcaccgtggattcattaccggtgactgaggcattggcatccgctccttcaatcagcatggctttcgcaaaacttcctgac
ggaatgtacggcagcgatggtccctgttttccctcattacgggtaaacaccttacgctgtatccggttaggcaccggcacagactggtacgtcacctgt
ggtggtggcgtgacaccattcccgggataaaacgccgtcggcggtggaacagcaacgctgccctgcggcgggaaggatacgggggtgtttcctg
gctgtggttcgccttccggcccgggcggtgaggctggcgtctgcggtacaggctcaccggtcgcggtgacgggatttgcacccagggcttttacctgc
tctgccagggcagcattatcctgtcccagcttttcaatacgacgctggtcatcaccgcgctgtttgttcaggacatccagttcacgacggatttcctcata
ctgcttctgcatctgcgctgccgtcacctgcatctctgtggtggcatgctgacgcaccttgtcatcaaaggtcgtatccaccacaccggtcatatccggc
acaggctcctgttcagccaccgcctcaccgttaccggccatgtccacatcagacagatacagtgcaccaccaatcgcggaggctgcaccgacaa
ccacaatccccagccacaggtactgcttgcgtttcacaatggtattgatactggccatcagttgccctccccgtcacggatgacatacacatccatgcg
cgcacctgccagtaactggctggcaggctggctgaacatcacggcacgggttcccggctgccagaaatcactttcccgaaggttattcaatgggca
gacagcgttttattttccacggcaaagcggaccatcttcaggtgattccccgtccatacggcatctgccactgaagacagccccgccggggcctgca
gggtttcctttgtcacaggaacctgataccagcctgcaggtaattttccgccacggacagcctggctgatggtcacaagcagggattcgtaaggcgtg
gacgtttcccacgcgccggcttcttctccggttccgcgcaggtcactgaccagctgaatggtacgccccgcaccttcacggggaacggcctgaatgg
aaaggttcagaccacgttctgtttccagaatgaacgtaaagggctttttgttgacggtggcaaccaccacaccgccggaggcggtctgctcattattgg
tcagggcaccatccagactgttcacggcgataatccggtcaccgggaacggtaaacagattcgggctggtattgctgatggcaatactggcctgac
caccgttcaccatggcacacacgtgggggccagcgtaccgtttgcggccatcacgctgcangaaaaaaacagctgccgaatattattgcccgtata
tttttctcatcgtctgtttcccgaaattctcagcagtcccccgttttcccgctcagaataggaagtaatgttggattcaggaaaggtttgatccccatccggt
tttaagccccccagaatttcacacggcatcggaggcatacccggccctgttggaaaaagctgagttcagttatcgttttaatacgcttcgcttcttcagcc
agattaatttcatctggttctgtgcgcccgggcggatatattgcagaagcgcttgatgtgaggcatcgacggtttctgatgaaacattcagacggaggg
caataaatgacagcgccatctgctgtaaataagaggcgtcggcactgttctgtgacacggcaaagggcacattaaatcccattggggtcacggca
gtgcgttgctcattctgaagacggtagttattcaccccctgaatgacgttaacagagaggctgagaacaataagcactgacataaatataaaggcga
tggccattacacgactggtacttaaacgggcaccgtgttccatataacaatcctggtatcagttctatttaatccactgccggaaacacgaatcgggaa
cattatgaaaaataccgcgcagcagggctgttggcatataccagtaaatcaggtcacgtaaccagtaactgcctcgcccttttttcagttttttaatcccg
aaataaaccagaaccgctgcaccaataccgaacagatatttcgatgttgtgataccccagccaatacagattgcggggggatcagttcatccagt
ggcagggcaaaccagcggctctggttggtcagtgtttccgggaaacgatatttcttaagtttattctcgtctcccgacatcgttttatttcctgtcagaggcc
aacgacggccatacccacagcaataaatacagagatgatggcaaaaccggccaggaacttgacgtttttggtcatcatgtacatgaccgcaccga
ccagaacttcagccagaacaacccatttaacaacactggagtccttaccgaaggtcgccttaaccgtggagttaccactctgcatcaggtcctgacc
tgccccctgggcggccagtgccagttgcgggaagaacatcatcagaacagcagcagggatcacagcgcgagccaggcgaagcatattcagac
gagtgaatttagaaaaaacgatttctttttgacgggcgcagaagcaccctgaacacttaaaacagtattcatatgattcctctgtttaatattgatagata
aaaactaaagttctttaaatgttgactcgttctcttcgatcacctccctgaatatttcttcattatagaaatcaggaaaccgtttcagatgatctctgagacg
gatctgaacctctatagttttacttctaccgctacgatctttggcccgaataagtcggttatttgtatcttcatcgagatacactgagactgttctgcttatc
ccgcctctcgcgttacgcctcctcactcaattcacctctgtttcccgactaataaaatcatgattgcacagaaaccgacgaaaaaaaacacgccaaaatt
ggcgtgccttatttaacatttacttaacaccataaaattcacgaattatacttaatagcgaatctatttctcttttatagatacatgacactctgaataaatc
aaagtgttcaaaaaattcttttattttccgtttaactggtttaattcgatcctcagatgctctgtctgtaatacccaactctctggcaattgttgcgtgacca
acacccagcatataaaaagagaaaacaaccaatgtccctggagaaagatcattccatatcgttaatttatcagaagtatttgtattacttccactctgattta
acgcaaaaaatggataatcaggaaacggattaatttgccataaaacaacagattcgtcattataaaaagaaacattctccatccttatctgatagatttcg
ccatgaaaattaaaacgacgacaaaatgcagagccatgtccaagagcctggcattccagttcaagtgatgaaagaaaaatttcaacctgcagac
gtatataactctctccggataagggtttatcttctctggaaaagagttcaataaaagccccattggcataaaggattttccggtttttgttacggacacaaa
ccggaacgggagaatttctgtagatattctgaacgttaaggatgtatttatttacagattgactgttaagtggcctttctctacgatccagcgcacacatag
gaacctccgttaaggattctatggacagtcgatgcagggagttcagacctccctgcatcggcgattttcagtttcaatttttaacgtggcattaaccacgt
aattaatataacctttatgcggttaattgtcatcaaattgaaccagatcaaaatcctgttatttgaagcaaatcaacttttaattcttttttattcctcatca
ttttctggaaagaacctctccatctcagatgacaccttatccctgatatcttcgaccatattcgcatattcaaactttggattcccggaaacatgagggctta
gtgactcaatacctaaaatcttagcaactgttgactgtgtttttaccgcacattcaagaagtaccttattaaactcagcctgattgaacgctgactctttacg
ctccatctgagcctcatatacacgaagccctaattcgagaagcatagtggatatgcttgagaaacttacatctgtacttttagctccttctgcacgtcttctt
tccacaattttatttattttatagacaatttcgtcactgacataagcctgcactttcgccatatgtatacctctttattttcttatcgcatcaaatttatttc
acatataaaaaaatgacaattcatcgatgaatcgaatcgtgacgcaagttacgaaaattacgattcatgcagcaaatcacatcacgaattgaatctagagtca
attcgctttaactcgttgttttattattactgattcatctatgagttgcgttaaattcattggtgaatcatatgcgattcaccaatgaatcctttttataatg
taaaataaattaaaatacattatttaaaacataagttaatgattcaaatagcaaatcagcaaaaacttgtttttgcgtggggtgtggtgcttttggtggtgag
aaccaccaacctgttgagcctttttgtggagtgggttaaattatttacggataaagtcaccagaggtggaaaaatgaaaaaatggatgttagcaatctgcctg
atgtttataaatgggatctgcgaagccgccgattgctttgatcttgcaggtcgggattacaaaatagacccggatttactgagagcgatatcatggaaagaa
tcccgttaccgggttaatgccattggtattaatccggtaacgggatatggcagcggactgatgcaggtagattcccagcattttaacgaactggcccgc
tatggaattaagccggaacatctgacaacagatccctgcatgaacatttataccggtgcttattatctggcaatagcctttaaaaaatggggagtctcct
gggaggccgttggtgcatacaatgccggattcaggaagaccgaacgccagaaccagagacgtcttgcctacgcatcagaggtttaccggatttat
accgggataaaaagcagtaaaggcatccgggttccgaccacgaagaaatcactttcccaaattaacagtgtgcagaacaattaatattgtatacatt
gtcaaataccggttatgccgcgacagcggcatggaaggccacacacaaaaatatcatatgagtgataatgctgactggccgaagccccgcaaag
cgggaccccatcttatatatatctggtaactacgcagcctcctgccagaccatttttcgcgggcacaatatttacagactaccgaaaagccagttgatg
aaattgaggcatatcagacgacacagcaatgccgcaggatgataatataagtaaagccccgtaaaaattttcggggctttacttatattatcatgactt
ccactccctgaactgtccggcaatcatccataatgctttgttgattcggatatcaccgtcaatgcctgtaatggctcttgttctggtatttttaccggaggca
cttcgccccgataatccaccttttatcatattctcctgaacccgctgccaggttgtccagagatcgttctgtttatcctcccaacggcggggagtaattattt
gttctggcgtcactggcgttttattttcatcttcgtacctggccatcaatgcagctctgccaaataaatgttgttcgtcactgttaagatgtatctctttcat
tgcctccatattttcagtgactttatcaaagacaccgagcacttcatacgctccctcaataacctgcccgacaatatcacctttatgtggaacgcggatttcg
ccaaaattattcccacacaccaggccatttgtgcagacaaaacgaaaaattcctgggatcatctgataactggatgaaccatcatgtgaattaagcagg
ataatttcaggaacttctttcccgttaatatgcccttccctgcgaagacgtaacatatgtttactgtattcgcggcgtcctaaatcacgaacccgactctga
caggcaaagaacggctggaaaccttcatcacgtaacttattgatgatattgatggttgggatatatgtataacgctcactccgggattcatgtttatcacc
agaaaagacagatggaacaaactgcattaattcgtcatccgttaacgggcgctcacggcgaatagaattatatcgaccaaaacgggaagctaatc
gcataacagtaactcctttatgaaggcataaaattaataaaagttttttcgcttaatatcatccccctcggttatcgttaacaaaaagacaacaattccga
ggggaaggaccatcaaatcaggtcagttcgggactgaccaacctgccgagtgaaaagcagtaacagcgcgatcaactcatcagcgttttttctgac
aagaggagtatgattctccaggctatccagtaagtcatgcagctcattattgacctggtggagcaccttaaagagtgcaccagaatcccggcagctct
gatatgcaagatttgcctgaacagcacgattctgcatgatcccgatttgtgcgattgcgtctttctgctgatcgcctgttggctgataagtggtgttaaaag
aaaatgcgctcataattcctcctgtatcgatgttttcagcccccatcatctgttgagtttcgggagccgcggttttgacgttttgcggggacctgccttttgt
tccctccggtattcaccatcacctgtttgaaaaccttcgccgcaaggggcatcactgccacggcctgacgcacctgtcctggggagaaagcaaaaattt
ttaccgggttttgggtgaaaaaatttgtgggctgttcatttaccctggacagtcagtgcggcaggttgtggctgtctatgcccccggcgacaggggctta
aacaggtgaatggcaccggacagaacaacgggcagggacccgccgcaacgacgtgaagcgcggtttgcgaaacggcgttgcgccttacatcc
cggcggccttttgccgggatgccgggagcgcaggccacggcgcgacagaggcggcgcgaccggaacggtcgctgcaagcccggggttttcac
gcatcggaccgtagcgacagcccgaaaccgcgccagcgggttcggcggagactggcgggtgcgccagcagctgcctgtcggagtcgggcctg
acgaccggatgccgccaggcagccgggagcgaaggtgcgcccggcagacaggacaacactgcagtagtcagaatcagagcggcaggaaa
aagaagagcagtagcagtagcagtagcagtagcagtagcaggaacaacaacatcatcagtaacagacaaatgacaggcatgaaaagacag
aagcacacagcagaacatccgcgtaatgaaaggggcggcgcgtaagcgcggcccctttcagcgccaccggcgtgctgtcttttcccggcgcggg
cagacgggcatttatcctcccgcctgcccggcgtcttcaggcccgcagacaaataatcatctcggggcacgggtataagcaaacatgccgcgcga
cagctccccgggaatgcagaagacgccagcaacgtcttctgctggcgtgatttgttcagacctctgtcgaaccgatccatataccattcagataagca
tcaaatgcatcattcccctcctgcacttcctgcggctgaacaaggttcaggctttcgtgtgtccagccggcttcaggtgcagacactttcagcagaagat
gcccatcagaatcacaaatctccagatcagcaaacgtgttcccggtcaggatatctggttcaccgggtcgttgatctgtaacatatgtgatatatcccat
ccattctctccacattaaatatttcagcactatccgggacgacggtgcggcccgattttcagtattccagagcaagttcagccatcagagcatctttcgct
tcccgtgccagcctggtttcttccatcgtcagaaaaagcccctttcgtgcagcagcaccaatgaataccggacgatccttcagaggaagccgggaa
agatgattcatcgccatatcaaacaaattcgcttcataaatttcatagtgcagaccacatccccaatgagcctgcttttccagcctgctgatcaccacat
cagcccggataacatcaaagggattgcgggaatgaatattacgaataaagccatcggtacgtgctgctgacatatttctgcccatctttaatacacctc
tgttttttgcccccatcatcttttgagtttcgggagccgcggttttgacgttttgcggggacctgccttttattccctccggtattcaccatcacctttttga
gaaccttcgccgcaaggggcatcactgacacgacttgacgcacctgtcctgggcagaacgcaaaattttttaccgggttttaggtaaaaaattttgtgggctg
ttcatttaccctggacagtcagtgcggcaggttgtggctgtctatgcccccggcgacaggggcttaaacaggtgaatggcaccggacagaacaacg
ggcagggacccgccgcaacgacgtgaagcgcggtttgcgaaacggcgttgcgccttacatcccggcggccttttgccgggatgcagggagcgca
ggccacggcgcgacagaggcggcgcgaccggaacggtcgctgcaagcccggggtttcgcgtcagggatggaagcccgccagggggacgcc
cggaaaagacacagccaggagaacaacaaaacagacaataatcagggcagcagtaatcagagtcagaacaggaggaaaaagagaataat
caaagggaagggatgtgatttctgacaggtcataaacagagaaggcgggggaaaagaacgggcgccgcaggcgcgcgttcttccccgttcccg
gagggaaacggccttcctggcagtctggttgttcatgcccgcacaatgccagcaaggagaaaggggccaccggcgaaccagcagcccctttata
aaggcgcttcagtgcagtcagaccagcatcagccctgaaaagggggcctgcgcccgcctccaggttgctacttaccgccggattcgtaagccatt
gttgccgtaacttccctgtatccgtccttcagccggacttcgcacaggcgattccgggtcaggagtgtgaatatcaacaacgtgcagcacacgattaa
tacgcaccagataagggcgtttcccggtagtttcatggtttactcctccttgcaatttgcacggtaagaggccatactggtgttggttagacatacagtgg
gcctcgtgggttaatgaaaattaactacggggctttctgctttctgccacacaacacggtaacaaaccaccttcacgtcatgaggcaaaaagcctca
agcgccgggcacatcatagcccatatgtctgcaccctgtcagcatctgcaccgttctccgtcacgccccggatttccagccgacgcagagcactga
acagataatcgcattcagcctgtttgtttgccctgaacggtttcgcgaccgtaaccacccactgatgatttccgggccagccatgtactttcctgacccg
cccgttaatcgcatacagcaacccccagcccgccggtaaatcggccacggtaatcacccccggctcgcagagataaaaacgccagtctcccatg
cccttttccggctctgaccggaacacctttttccggtcagccaggaaatcagaccgtgagcatttcacctcaatgagacatgacgctccgttacggaa
gcccagggcatcaggatgttcaccgcaggacgtccatgcgtgaaaacggtcactgaacaccaccttaaagccgttgttgcggagaaaacggcag
gccagttcacaaagttccctgtgtgttttcgggtgtctggcgttctgcatcacgccacctcccggagtttgttcgggaccatccaccgctccatctcacgg
acagcgtgaccggcctcccttgccgccataccagcctctgcgatgacctgccagaatggcatatcctgccactgccagcgttccatcccggtcagtg
acggaaaacgctcgccccactgccagaacagggtttccaggtcatgctctgccacccggtcactccagcgtgcatgccagcttccgactgtttccgg
cgaatggaatgccagttcgatttcagcctgagcgaccagcgcctggtacgcacgggctttctgctctgcctccctcgcctgctgacggctgtacttctgc
atgaccagtgcggatttatggcgactgcgttccctgacacgaaaccgcaccgacggaaacaaccgctctgctgcatcaccgggaaggggaagg
aggcagacctcgccctgtgtttcaatcagcacatcgacggcatacagccactgaagacggttgccgtgcagttcttctgcggtcagggacggcatg
aaaaatcgcaggtcagacagcgatatcctgcggcttccgttcagacagcggaaaaacgcccccgcatacggatattccgccagcgtgttaccctg
atggcggcgtttttctgtctccgccaccgcctgccatgcggcctgttgctctgtgcagagaggaaccagtgcctgtgaacgtacgctcatgcctgacttc
cttccgccatcaggatatggatgatgtcattaacgctgtacccctgcgcatccagtgacgccgccagtgccagtgaatggctgacagcttccggtgca
aaccgccgctgcacctgaaccaccgcaaacggctcaccaccggcattcaccagaaccagcacccagacctgagagacatcgcccggtgaac
ataacgccaggtgaaaacgttcgtgggcaggcgtgaaacggacctggacggggaaaaatccgccaaactcgctgccgtactcgccgttcatcgt
ccagttgtccggcgcatccagttcccgcatcacggcgtgtgtcagctcacgacgttcatcacttccggcagcccggatatcttcatattcctgtgcgctc
atggtctgtaatacattcagggtcagttcagttttcatcacagtatctcccttgctgccgccccgtctccggcggggcggtgtggttgttcatcaggcggc
atcagccaggttatcagtgcgttcagtggtatctgtctgtggatgtggtgcacacatccagccaggcacccagcggttgtctttcatatggtgctctgcat
gttcagccgcatcacctttcttcatcttctctgcatcccgtgcggcaccggacagtccggcatcattcagggctgcgataatctgcggctttttcaggtgtc
cgaagaagtttgcttttgtcggctgccaccagtctcgcatgtgaaagccgatggcggtttccagcgtgtcaagcggactgcgtgacgtgtgaccacac
tcacgggtctggacaccgttaaggctgcatgcggtgcagaaactgagcagggataacagcacctcctgactgagtgacaggaatgtcgtcatgtcc
cgtgaccatccctccggtaacagggcagcaagacgggctttttctgccatcagcgccatgaatgcggcaccttccttccccgatggcgcatcgctggt
cagcgaataatgtttacattccaggctgatttgtgctggtttactgtacgctccgctgccaaacacattcaggcagagcgtccatgccagcagtgtcag
ggatttgtccggttgctgcatcagtgctgcctggactgccagcgtgcgttcggaagacattttcgtcagcagtggcagactgatttcctccactgacgctt
tctcctgcacctgttccgtacggtcagcgtcgtcagccacgtcatcctcactgcgcaactgcacaccacgctggacacacacgttgccataacgcca
ggacaccaccactccacctccggcacgcatctccggcgtccacgccctgaccttcgccatgcagcgcatcagcttcatttctgcttccagcaggtca
gattcctcacactggttttccagcgcgtcgtaacgcgtcatcagttcgttcagacgttcttcttccgcctccgtcagcaccgcttccggctccggcagaca
gcggtatgttccggcatcctcacggcactcaccgacaggctccatgcgtccggcgcaccattcccagccttcggcttcccgaaggaactctgcgac
agcctggagtttttccagcagggcggcatcgagcgccacgcagtccacatagccaccttcgtcgtcgctgaacaaatcggtgcgcagttcgtctggc
gagaaggcatcagcccccacgaagcggaatttactgttccccgccaccgccacttcactttcggtcaccagacgacgaatggtctgtacttccggttt
accgccccatcccgactgacaggcggcttcaaacacctgcacctgacgcgcggtgtcgttctccagcgccagcgcctgacagtgttcggtggtgat
gcggtcttctgccagcgcatcgaggatgacaggcgcaaggtcagccagtttcagcattcgctgaacgtggcggggtgaatagcccagcaaatcac
cgatttgtgcaggtgttttgccttcctgcgccattgcgcggaatccggcaatctgttcggcagggtgcatatcccgacgctgaccgttttcagtcattgatg
cagccgtcgccagctcctgcggaataaccttcacgcgtacaggccagtcagccggaaggatgccacgctctgccagcatgttgagtgctgccagt
cggcgaccacctgcggcgacaccgtggcggtcaccaggcagggcatgaacaaccagattctgcaacaggccaacgcccttaatggactccgcc
agttcgctgacggactctgcggaatacggcaccgtgcgcacattcagcggtgatttaatcagcgaggccagcgacacgctcacttcctccgtctgct
ccagcagggcagacaggactgtttcctgcgtttttgcaggtttacggctggatttacgctccgtttttgtcttagactctgtaactgacatatatttctcctt
tgaccgggatggttgtatgtctgccggacgcgttcccgtccggcacctctcttacttaagaatgccgaaattccggatgacatggttcatccagtacccggc
accgtcctcaaaattccaggtacgccagaccaggccactgttattgcggacaaccaggcgaaaatgcgttccctgatcatcctcgataaagacattc
cggtactgtgcggcaacggcttcagcctgttcgcgggtaaaggagccgtcaggcagcccctgaaatattctgaaatattcgctcatcctcatatctccg
gtgatgccccgcactggacggggcggttgtcactgtctttcgttagaacggaatgtcgtcatcgaagtcatacgcgggttcagacggctgctgaggct
ccggctgcgcggccttacgtccacggcctttcgttttcgcgccaccttttttcggttccggctgtgattccggctgaggctgaccgctgaactgttgcgcct
cttccggctgagtctgagtacctgcggcacgtcccagcatctgcatggtgcccgtggtcttaacaagaatttcagtgacgtaacgggtgataccgttat
cttcccagctacgggtacggagttgcccctcgatgtagacctgcgcgcccttgcgcagatattcaccagccacttccgcgagcttgccgaacagcac
cacgcgatgccattctgtctgctcccgcatctcccccgtctgtttgtcacgccagctttctgacgtggccacctgcaggtttgccactgcgcccccgtttgg
gatgtaacggacttccggatcttttcccagacgaccgacaagaatgaccttgttaatgccacgaactgccatgatgtgtctccttctgttgatgttttcagc
ccccatcatctgttgagtttcgggagccgcggttttgacgttttgcggggacctgccttttgttccctccggtattcaccatcacctgtttgagaaccttcgc
cgcaaggggcatcactgccacggcctgacgcacctgtcctgggcagaacgcaaaattttttaccgggttttgggtaaaaaattttgtgggctgttcattta
ccctggacagtcagtgcggcaggttgtggctgtctatgcccccggcgacaggggcttaaacaggtgaatggcaccggacagaacaacgggcag
ggacccgccgcaacgacgtgaagcgcggtttgcgaaacggcgttgcgccttacatcccggcggccttttgccgggatgcagggagcgcaggcca
cggcgcgacagaggcggcgcgaccggaacggtcgctgcaagcccggggttttcacgcatcggaccgtagcgacagcccgaaaccgcgccag
cgggttcggcggagactggcgggtgcgccagcagctgcctgtcggagtcgggcctgacgaccggatgccgccaggcagccgggagccgaag
gtgcgcccggcagacaggacaacactgcagtagtcagaatcagagcggcaggaaaaagaagagcagtagcagtagcagtagcagtagcagt
agcagtagcagtagcaggaacaacaacatcatcagtaacagacaaatgacaggcatgaaaagacagaagcacacagcagaacatccgcgt
aatgaaaggggcggcgcgtaagcgcggcccctttcagcgccaccggcgtgctgtcttttcccggcgcggacagacgggcatttatcctcccgcca
gccctgtgtcatcaggcacgcaaacgaataatcatcgtcgggacatcggtataagcaaacgtgccgcgcggcagctcctcgcggatgtcagaaa
acggtaacagcttctcctgctggcgcggcccgttcagacagacggcaaccagcacgccccccggacgcaacagggaaaaagcccgcaggat
atgccggatatcctgcccgtggatgaacggcggattcataatgatccggctgtaatactgcaccggctgccattccatgaagtcgccacactgtaccc
tgacaccgttaaaattttcccgcagataccggaccagcccgctgttaatctccaccgcatcacacatggcctccggcgcggtatcccgaatcgcccg
cagtatggccccggtgccggcagagggttccagaatgtgatcacggttgctgatttccgccagcgtcaccaggcgatcacagaccggggcagga
gtcacaaaaagctgagattcagcccggatttcagcaaccggcgcggcgttatccagccttgcgcgcatcagccggatacgttctcttaactgactgtg
catgtcattatctccgtcatcccccggggatttcccggggggatataccttatttctgtgggatttccgtgattttcatgtcggtaatatacacattcaccag
gcggaaattattgtccattgtgcggcggtagcggtatgccccgtggtcttctgtctctgccacactgcgcacggccttacagtcccggggcagggcggc
ccactgcgcctttgtcatttcccggaaaccgtcccccggatagttgactaccggcggacgcttcgcggcctgactggcgacggcagcctcttctgccg
atggtgctttgtagtccgtgatgcgatcgggcgtcactttcatcgtgccgctgtacccgagaaaactgtaattcggcgttgtgactgaactcaccgcccc
gttgcttttgttcacgcggatgatggtcagccactcgccccggctgaaaacctgtccgcccggctcaaaatcctgtgtccgggtaaccacgccgccgc
tttcgtccagcatggcacgctcatagatcaggcggttctgatagtggttaacccagcgttgctgatgctgaatctgccgttcatgacagcggatcgcaat
ttcgcgggcctgctccgtggtgatgatgtcatcatccagggccgaccataacgatcgacttccttcatactggcttttttctgccgggcgcggatacctgt
ccagcgggaaacaggcgctgatatggtcatgactgctgatgagttttgccatattcagatccagcgattcagcccgccacatcgtcagatatttctgcg
actgtgcgatggtcttttccgccttgcgcaaatcagcttcgatttttttgatacgacgccagcgaacgtccggacgttctttatacttcgcgtgaagcagtg
ccgaccgcgcccgctcttcccagtattccgcacgttcaaagagcatcacagcacgtttcataccgttttcaatacgctgtgcatcacgacgggcgcgg
cgttcgctgtgatgccccacaagaatgggctgaccggggggatcatcgcggccagtctttccacttcatcaagtgcctgtgcggattcactggcccg
ctttccgctgtatccggtaaaccgctccgcccgtgcttcctgacgttcagcgagcgtgctgtcttcatcctcaatctctccggcaagtgagaggagcacg
tcttcccggcccggtgtccaggctggcgcgacaaacagcgcctgtttgggtgcccagcgaaaaccggcatcatgcacttttttatacgtctccgggtc
aagacgtgacgcggcatacaggcgcagtttgttgtcatccggtgagtaggtcgcgcggtacatctgcacagggctggcggcttccggggcggtcat
ggtgattgctgctaatgtcataaaatgttctccttccgtctgttgccgcccctcgcggggcggctcacctcatcagttaaaagcgcgtttcagttcgagtgt
gcggagcacctgcccgaaggccgtgtttttcatggcggggatgtgtacaccacgcccccggtgaaaactccgggctatgtgtcgcccgaaggctttc
agcacggggtcaatttccggcgcggggatgtcttcgcgtctgaaacggggatgacggcggggctttacagccgctgcggaaggtgaaacaatact
tattgtcataatttagcctcttatgacgattgactggagccgcaaaaactgttcgtgcagtctctgcggcatggattaagcaggttcctgacctgcttttttc
atatctgcatctgacgctttccggtgttttcctccttctgttcctgcgttctcattacctgccagccggatgactgacagaacacataaatgtgtattgccgg
agcctgaaataaatcctgatttttaaagaacatcacccgttgagtttcgggagccgcggttttgacgtttcgcgggaacctgctgctgtattccctgtggtat
gaacttcacccgtttgagaaccttcgccgcaaggggcatcactgccacggcctgacgcacctgtcctgggcagaacgcaaaattttttaccgggtttt
gggtaaaaaattttgtgggctgttcatttaccctggacagtcagtgcggcaggttgtggctgtctatgcccccggcgacagggactcatacgggtgtgt
ggcacccggcaggcaatgagcaggggcccgctgcaacgacgaagagcgcgtgcgcgaaacggcgttgcgccttacattccggcggccttttgc
cgggatgccgggagcgcaggccacggcgcgacagaggcggcgcgaccggaacggtcgctgcaagccgggggttttcacgcatcggaccgta
gcgacagcccgaaaccgcgccagcgggttcggcggagactggcgggtgcgccagcagctgcctgtcggagtcgggcctgacgaccggatgcc
gccaggcagccgggagccgaaggtgcgtccggcagacagaacaacactgcagtagtcagaatcagagcagcagataaaggaacaacaac
aacatcaggaacagttccataacataagaaagaaacactcctgagtcagacacatcacagacgggaagggtaacgggcgacgcgccagcgt
ggcccgcttcccttccggcaggcgtcagcctgccaggggttttcgcgcgggctgacaggcggcctccaccgcctgcccctgtatcacctgtttctgctg
ccctgtaagatgtagtgaccgcccaggaaaaccgcctgctgattcgccacaaaatcgcagatttcatcaacaatctgcgcggtggtgagcggtttct
gccccatttcacgacgccgcgcattaatcacggccctgtaattacgcagaatttcgcgggtttccgtgctgatattcatgacatcgtctccccgtccagc
gcctgcaacacaacgtccattgcttcgcgttcctccctgacgaatgcgtcaccgtcgcccccgttctcactcccgacggcatacaggcgcttcatttca
ctgcctgccagcgccatcactttttccagcatcaccgccgggactgtcacctgacggctggcgtgcttttcctccatcagttcacgaacaacgtcgtga
acaacgctgacatccgcaccatgctcaaaggcatcgtccagccgctgcatgacggtttccagctcatcgtctgtcagactccagtcctgggctatgtg
acgcacatccccacgggccagcaccacggcaaaaacgcattcactgtccggcacgtctgtatccagcacttcgcggataatttctttaacagtaca
gtacatgttctcgtctcctttacaggggcggcattgccgccccgtccgttaatcagtccagcgccgcataaatcgcgttacattcggggtgaaattcgat
gtggctccacaactgcgcatcgcgcatcctgtacaggtgagtcagccctgcgctaccgctgtcgtggtacagccacaactggcggttaatcacaag
ggaggtcaggatgatgcctgcggcatcagcactcacggtacggtcaaaccagttttcaccgttaaccagatgaaaacggtcgccatccggcatcat
gtaaccgccaccgtccggcaggcggatgaagtgccagaatccgccttcatattccggcatcatttttttcgccagcgcgtacacagatgcctcggcat
acagaaaatccaggccaaacaggtaaggcagaaaggagagacggtcgcactcttcaacgttcagagtgacaggtttcgcatattgcatgatgtttt
cctccgttgttcacatattccgcgacaccgggcggtgccgtgcctccatctttgccgggcctgcagggttgcaagggcgcaagggaagccgcagcc
tgacgcacacggctggcgggagttggagggtgagaccgcgaaatctgagcgggtgaacgaccggaaaatcacggcagacgggaaggaggg
accgggtgaccgcagcggttcaccttaccttgcaacccgcccgcagaagcccggctacgatggaggcacggcacagcccgcacacgacgctg
aggccacataactgaaaacccacaaggccatgccccgcatggccgggtaaagcggagcctgcggcgacatccttttgctctttccggggaccgg
ctggaaaaggcggagatttttccccgccagcgtcatcatttccggattttcgccagtgcatccgccatgctgcggatttcatcccgtgcctccagaaca
acctcccgcagaatatcccggtcaaaccagcgacgggacggttcatccggtatacgcagcacatcacagaccagaccactggacaggatatgct
ccacacactcactgaccagatacatttccaccgccgaaaaacgtgctccccggttcaggatagcatcgagcaccacgggcagattttcgtcatcttc
atcaagccagcaggggtaatcgctttccttcacaaccagacgaaagcggcgcgtctggcgggtcagcaacgtctgaccggtggtggtcagcgcc
gcacggatacgccgcgcatcttctgccacccggcaactgacactgcatactgcatcgcgcaccgcatcaagaaccgccctgatttcttcctccgtca
gccattcaccggcatacgggaaagacacctgcgttccgtctgccggaagtaccggcagcgactgccactgactaccggacagaacaagttccgc
tccggttccggtcactttctgaatggcagaggccaggagcggcggcagtacgccgccgtgacggttgataagtaacgtcgccagccggggcgcat
ccagccgcacgtaaatgctgatccagcccggatagtgcggattcagttgttcaatatccgtctcttccggccagccctgcgccagcagcaggttacg
ggcgtggcgataaaccagtgcattcagcgttttgttcatgattaatggtctccctgaatcgccgcccgaaagcggcgataacatcagtcgttttcgtgtgt
gttgtttttcagcagggcgcgggcctgattaaccaccgcctgacggcgggtgacgcattccgggacggcgaggccatgatcccgggcggcccattc
acgtttccacagtgcctgttcggcggtctgaatcagggacgccagcagttcagccgggacggtcacctggggttttctctcacgttattgatgatctcc
atcgccacgccggaagaaataccggattcagtccgctggtcttccgggatgtcctccaggcgcgccaggacagtcctgatttcatgatcggacagg
gaaatatccattccgtcagcgagggcctgaatttcttccggggaccagataaccagcatcagcggcgtgtctcccggatactgcgctgccagctccc
ggcatagcgtttcgcatgttccgtacattctcgtctccttctcaatctttgaagcaggaatccagttccagaatttcggtatacgcctccatgaacaggcg
attggcgcgatacagcgtctggcgtgtcaggcgttcaacaagcacatcggaatcaaagcgacagcgatgctccatatcgttcagcctggcccgaa
ggtcacacatggtgttggccacctccgccacgtcggcgcgaagtttttcgtgtccggcatagttcatttacgcagcctccggttcaaaccagttatcattc
gcggccccctgctgcatggcccgttgcacggcggccagtcgttgctgtccggcacggtgatactgttcaagcagctcgataccgatataccggcgtc
cggactgaagggcggcgacgcaggttgagccgctgcctgcaaacgggtccagcacaatcgcgttcgggtgtgtgaagctctcaatcagcggttgc
aggctggtaacgggcttttcagtcgggtgatgacgattgcctgaatatttccagcccagcacgtcgggtaatggcttctgaggcagcgccggacgtcc
ttttgccaggatgtaggcgcattcgtggcgatagcccacatatgcggccttcgatgtgtaggttttggtaaacaccaggtggccaaccacgctgaatcc
cgcatttttccaggcggtcataaagcgatcgacgcggttccagccatagaagctcaccattaacgcgtcttttttgagtacgcggtacatttcattacag
gctggttgcagccattcatcggttttatcgccggcgatggtgcgcccggaacggtcacggaaaccgacgagatacggcgggtcggtgaggatgaa
atcgacagcattgcccggaaacgtggccataacacgcacgcagtcacccaggataaaacgggacataacagcactccttctcttacagagggca
ccgggagggcacccctgtggtgcccgttccccgctgcctgtgggctgttcaccgcgtccggtaacgggtcaggccgaccgtggaataccgcagcc
gtcgtctttttgacggcaaggctatgccgcgaaagcggcttgaagggcgttcatcagaaaagggctcccggctggcgtacactgtcaccaacgcca
gccgggagcggtaaaagcgcccgtcccgtgcccggccggacgcgggacactgaccacaggcagcgttcctgccgcgccgcgtcagccggcg
cagtaaaaagaggagcccggcgacatcttttgtcacccgccaaacaaaacccaaaaacaacccacacccaacccaataaaacaccaaaaca
agacaaataatcattgattgatggttaaaatggggtaaacttgacaaataaacccacttaaaacccaaaacatacccaaatacacaccaaaaaaa
caccataaggagttttataaatgttggtattcattgatgacggttcaacaaacatcaaactacagtggcaggaaagcgacggaacaattaaacagc
acattagcccgaacagcttcaaacgcgagtgggcagtctcttttggtgataaaaaggtctttaactacacactgaacggcgaacagtattcatttgatc
caatcagcccggatgctgtagtcacaaccaatatcgcatggcaatacagcgacgttaatgtcgttgcggtgcatcacgccttactgaccagtggtctg
ccggtaagcgaagtggatattgtttgcacacttcctctgacagagtattacgacaggaataaccaacccaatacggaaaatattgagcgtaagaaa
gcaaacttccggaaaaaaattatattaaatggtggggatacattcacaataaaagatgtaaaagtcatgcctgaatctataccagcaggttatgaag
ctctacaagaactggatgagttagattctttattaattatagacctcgggggcaccacattagatatttctcaggtaatggggagattatcagggatcagt
aaaatatacggagactcatctcttggtgtatctctggtcacatctgcagtaaaagatgcactttctcttgcgagaacaaaaggaagtagctatcttgctg
acgatataattattcacagaaaagacaacaactatctgaagcaacgaattaatgatgagaacaaaatatcaatagtcaccgaagcaatgaatgaa
gcacttcgtaaacttgagcaacgtgtattaaatacgctcaatgaattttctggttatacgcatgttatggttataggcggtggcgcagaattaatatgcgat
gcagtaaaaaaacacacacagattcgtgatgaacgttttttcaaaaccaataactctcaatatgatttagttaacggtatgtatctcataggtaattaata
atggacaagcgcagaaccattgccttcaaactaaatccagatgtaaatcaaacagataaaattgtttgtgatacactggacagtatcccgcaaggg
gaacgcagccgccttaaccgggccgcactgacggcaggtctggccttatacagacaagatcccagggcccctttccttttatgtgaactgctgacga
aagaaaccacattttcagatatcgtgaatatattgagatcgctatttccaaaagagatggccgattttaattcttcaatagtcactcaatcctcttcacaac
aagagcaaaaaagtgatgaagagaccaagaaaaatgcgatgaagctaataaattaattcaattattattgagttccctttatccactatcaggctgga
taaagggaacttaatcaagttatttcattagcaatgattacataaaataaatacagtgtcgttattataaaattatttcacggcacttataaaaaaacagt
accaataatcaaacctggtgagttcgcaatgtctggcaataacgtttttgatgggttgttttccagcccggagcgcgatattgccaccagtcacagaaa
caccgagaacaccagatttcagcggtcgctgaatttgaacgagatcttttacttgaacgaacttattcagggatagcaagagcacgggatgcagga
aaacgttttggtcgcccaccggtattaaatgaagaacaaaaacagaaggtactcgaacgcattaagtctggggcaagtataagtgctattgccagg
aaattccgaaccacacggcaaacaattctgagagttagagcgaaacagcaaactgttactgtatagaaaatagtcttagcgtgggattccgttttcca
agcggaccccaaggaagactacatcgcgttcatgccgaaaccgaatgtaagaacggcattgcataatcttgcagtggcgatcgaacattacaatg
aaaaccatccgcacagtgcgttgggttatcgctctccgcgagaatatcgacgtcagtgggtaacgttaacttaagatacaacccctgtctggaaataa
gggggcaagaacacattgacactgtaaatatggcattatataaaactccgccccaataatgagtttcatttgcattactcccatttgctggtcatccactc
gcctgtttacaaggatagcgtttaatgaagaaggtcctttgcgcgttaggcttgatgttcaccgccgtcagttccgcgctggcaacaacctaccccctg
acgattgaaaactgcggctatcaggagaccttcacccggccgccggagcgcgtggtcgcgctggggcaaaatacggtcgaaattctgctgctgct
cgggctgcaaaagcaggtggtcgccagcgccttctggccgaccagcgttttaccgcagctggctgagcaaaacgcgaagattaaaaccttgacg
gtggaaattccgagcctggaatcggtgctggcgcaaaatcctgatttcgttcccgcccagttgccgctgctgctggggccggaaagcaaagtcgcc
aggcgtgaagatctcgccaccgtcggcgtgaacagctacgtctcaccggggatgtgcgccaccaaaaaggccaccggcgatatgtacggcagc
cggcaaaagctgtgggatatgacctggctgtatcaggaaattagcgatttttcccgcattttcaacgtcgaagatcgtggtcaggcgctgattgccgatt
tcaaaaaacgtgaagccgacctgcgccaggagtttggtaaaagcaaaaaggatctctccttcgtcttctggttctccagcgcctcaccttcggccgat
gcctatgtcggcggcaaaaacagcgcctccggatttatcgccagcgtgctgggcggacataatgcgattacctccgaaaccgagtggcccacggt
gagctgggagagcattattgccgccaacccggatgtgatcgtggtcgccagcctggatcgtaaccgctgggcgctggataaggccgaggaaaag
atcaaattcctcaaaagcgatcccgccgtcagccagctggaggcggtgaaaaaaggccatatcgtggtcatggacggtcaggcgatgaatccga
ccattcgcacgctttacggcgcggagcaggtgggcgagcagctgagaaaaatggggctgaactgatgagcgtcgccgcgattgagacgcgccg
cagcatactgctcaccggctggtgtgtactggcagcgatcgtgctggcgctggtgatcgctgtgggcgtcagcgttggcgagctggccatcccgctgc
agaatgtcttttatgctatcagcaacagaaccggcttaaccgctgagccgctcaaccgtatctatgagagcgttatctgggatttccgcctcagccgcg
cgctggtcgccgcctgctgcggcgccggcctcgctatctgcggtgttgtgctgcaaagcttgctgaagaatgccctcgccgaaccctacgtcctcggc
gtctccgccggggcctccaccggcgcggtgtcgatcgtggtgctgggccttggcgctggcgcaatttccctctcggcaggcgcctttgccggggcctt
cgccgccttcgcctttgtcgccttgctgactaacggcgcgcgcggcggcaatgagcgtacgattcttgcgggcgtcgccgcctcgcagttatttaacgc
catcacggcctataccatcagcacctccgccagcgcgcagcaggcgcgcgatgtgatgttctggctgctgggcagcttcagcggcgtccgctggcc
cgaattccagttggtcatcgtggtggtgctcgcgggtctggcggtctgcctgtggtatgcccgggcgctggacgcgttcaccttcggcgacgatgccgc
cgcctcgctggggattgctgtgccgcgcgtgcgcctgatcctcttcaccactgcggcgctgatcaccgccaccatcgtcagcatggccggctcaatc
ggctttgttggcctggtggtgccgcacgtgatgcgtttcttctttggcccactgcaccgaactttgctgatcgccagcgcgctggcaggggcgatattgat
ggtactggcggacattgcgtcgcgcctgctgattgccccgcaaagtctgcccgttggggtagtaaccgccctggttggggtgcctttctttgccgtgatt
atctaccgctcaaggaataagtgatgagtatttgcgctgaaaatatcacctggaaggcaggcaaaaaggtcatcgtcaataatgtctcgctgcgggt
gccgcgcggcgaaacggtcggactgctggggcccaacggctgcggcaaatcctcgctgctgcgcgttctggcgggcctgcgccgcccggatgca
ggtcgcgtcaccctcgacggccaggatatcgcccggatggcgaaaaagcagctcgcccgccgcgtggcttacgtcgagcaacacggcatgacc
gaggccaatatgcgggtgcgcgacgtcgtgcgcctgggacgcattccccaccactctccgttctcaaactggagcgctcaggatgacgaagcgatt
gccgccgcgctgcagcgggtagcgatgctggagaaaagcgaacagggatggttaagcctctccggcggcgagcggcagcgggtgcatatcgc
ccgcgcgctggcgcagagcccgagcgaaatcctgctggatgagccgaccaaccatctggatatacaccatcagatgcagttaatgcagttgatca
gcgagctgccggtaaccagcattgtggccattcacgatcttaaccatgccgcgatgttctgcgattcgctgatcgtgatgcagcaggggcagatcctc
gccagcgggacgccggaggagattttgtccgaagcgctgctgtgggacgttttccgggtgaaaaccaaaatcgagatctccccttaccacggcaa
aaagcacattcatttcatcgtttaggttgggcggatatgcctcttttctccctgcgccctgcggcaaccctctggcctccggtgttactgggaagccagttc
gtttttaatattggcttttacgcggtcgttcctttcctcgccctcttcctgcgcgacgacatgctgctctcaggcggacttatcggcctgatcctcgggctgc
gtaccttctcccaacaggggatgtttatcctcggcgggacgctggccgaccgctatggcgctaaggcgatcattctcgccggctgcgtcgttcgcgtcg
ccggttttctcctgctggcgtgcggggcgtcgctgtggcccattatccttggcgcctgcctgaccggcgtcggcggcgccctgttctccccgtcgataga
ggccctgctggcgcgggccggtacccacagccaggccaacgggaaacgcagccgcgccgagtggtttgcgctgtttgcggtatgcggtgaactg
ggagcggtgatcggcccggtggccggaggcgtgctgagcgggatcggctttcggcatatcgccctcgccggcgcgggaatatttcttctggcgctg
gcggtgctctttttcggtctgcccgccgacgggcacacgacaacaacgcgcagacgagtcccatggtggacgcctttgcgccagccgcgctttgtcg
cgtttattctcgcctacagctcatggctgttgagctacaaccaactgtacctcgccctgccggtggagatccaacgttccggcggccgcgagcaggat
ctggcgccgctctttatgctggcctcgctgttgattatcaccctccagctccctctcgcccgcttcgcccggcgaatgggcgccgtgcgcatcctgccgg
tgggctttctgctgctgtcggcttcctttgccagcgtggcgttattcgccgccgccccacccgccgaaagttggctgcggttaatgccggcggctggtttt
gtcacgctgctcaccctcggccagatgctgttagtgcccgcagccaaggatctgatcccgctgttcgccgaggagtcgacgctgggtgcccactacg
gagcgctcgccactgcaggagggtgcgcggtgttagccgggaatttactgctcggtcatttacttgaccaggcgctcattccgtcgccgcaggcggtt
tatccctggcttctgctggcgctgtttccgctgtgcagcgccgtcgccctgcgggcgatctgtcgcccgctggcggcaacatgacgcagagcgggta
acagcccgctctggaagtgaacttcattgcttacggccttgaggccgtaccgtctggtgtgcgtgccattgtccaggacggtatccccccctcgcacg
gatatttcgccgctttggcttcatcaatgtcaatacccagacccggcttatcgttcagataggcataaccatgatcaatctcagggcagcccgggaag
acatcacgcagcgcatcgttcattggcgtgtattcctgaatgccaaagttcggtgaactcagatccagatgcatattggcacagaccccgaccggag
aaatgtctcccggcccgtgccatgcggtgcgcacgccgttcagttcgctgtagacagcaagtttcttcgccggcgtaatgccgccgatggtgctcaca
tgacagcgaatgtagtcgatgagcttgttgtcgatgagcggcttccactcattcacattcacaaacagctcacccatggatatcggcgtggaagactg
ctggcgcagcattttcagccagtcgatgttttccggtgccaccggatcttcaagataaaatagctgatactgttcgagcgttttggccaggttaatggca
gtcacaggggtgacgcgctcgtgcacatcatgaataaattcgatgccaaagcccagcttgttacgcaggtgatcgaacaaacgcgggacgcttttg
gcgtaggcatccgggtcaaagtagatgccaggcgttttgctgcgcggcgaacgcttcggctggatgtttttcgcccgcgccagctgagtggcgatca
gcttgagatcatcagtccccgcgccgccgtacatgcccatctggcatcgaacgtactgatagccctcttccattctggcgcggatattatcttccacctc
cacttcatcgccgccgtcggtgtggcagtacagtgggatcccatcacggcatttgccgcccagcaggtcataaaccggcatccccgccagcttacct
ttgatatcccacagtgccatatctacgccggacaacgcattgttcatgattgggccattacgccagtagccgcttaccgcgcccgactgccagatatc
ctcaatacgcgtcggatctttgccaatcagaaaaggtgccatgtactcatcgattgcgctttttaccgcgaaaatacgctgagtaaatgttgcacatccc
agtccgtacagccccggctcgttggtctcgacctttacgaccgccaaatcaataccgcccggcgccgtcagaatcgttttcacgttggtgattttcaggt
tactcactttcactcctcaactcagcgaatttaccgtatgtcataccgttgtggatgacatgacacatgtttgtaagttgtcctacaacttaatgctgttata
tcacgcttctttcgaagaggagcgctgcgagcgccattctgtgacagcgatcaaatctcgttcaatttaaattaaattaaacaatataattcaatgaattaat
tgaatttaagtgatcgccatgcggagctgtttgtgatccttgttgcaaataaatgggtgagttgttgattttatcagagcgaaaattcaaccttaaataaga
cagtcgtcatacaagttaaacgagtgaggaaacaacatgaccgctttatttgatttaaccgggaaaacggctctggtaacgggctctgcgcgtgggc
tgggctttgcctacgccgaggggcttgccgttgcgggcgcacgggtcatcctgaacgatatccgcgacacgctgttggccgaatccgtggatacgct
gacccgtaaaggttacgacgcacacggcgtggcttttgacgttaccgatgaacaggcgattgaggcggcttttagcaaactcgatgcggaagggat
cagtgttgatatcctgatcaataacgccggtattcagtaccgcaaaccgatggtcgagctggagctggaaaactggcagaaggtgatcgacacca
acctgaccagcgcatttttagtctcccgcgcggcggcaaaacggatgatcgcccgcaacagcggggcaaaatcattaatatcggctcgctcacc
agccaggctgcacgtccgaccgttgccccttatacggcggcaaaaggcggtatcaaaatgctcacctgctcgatggccgcagaatgggcgcagttt
aatatccagaccaacgccattgggccgggctacatcctgaccgacatgaacaccgcacttatcgaagacaaacagttcgacgcctgggtgaaaa
gcagcaacccctcacagcgctggggtcgtccggatgagctcatcggcaccgccgtcttcttatcatccaaagcatcggattatatcaacgggcaaat
tatctacgtcgatggtggctggctcgccgtgttataagccgtccgtactctacctacttgcctttaaaattgacggcatgtgttcaccttgatgcatgccgat
ataacacaggtattattatgaaggcttcaagacaacgactctttattctcacattgctgtttcttgtgactgcaattaattatatggatcgcgccaatcttgc
cgttgctggttcgaatatccagaatgatttcagtctgacgccaacacaactgggtttgctgttctccatgtttacctgggcctacgctgccagtcaaattcct
gtaggctatgtccttgatcgcattggctcacgtattctttacggtggcgcgattattctgtggagtatttttacctttatgatggggttcgcctcacatcatt
tattcgcgacggcaaccgcttcatttgtgatgcttctggcctgtcgcgcattaattggtgttgccgaagcgccgtccttcccgtctaatacaaaaataatcgc
cacctggttcccggaccatgaacgtgcccgtgcaaccgcgatttattccagtgcgcaatatcttggtctcgccctgctaaccccagccctcgcctttattgt
ggctaactacggttgggaaatgtcgttttatctgtccggcggcgtaggaattctgtttggcatctactggcttatgtattatcgcgatccccagcacagcac
cgccgtgaaccaggccgaactggattacatcaaggcgggtggcggctacggctcggccaaccagtctgcggttagcgagaaaatcagctggca
agatattaaattcttcctcagtaaaaaaacgatttggggtctgttcatcactcagtttgcctgctcgtcgacgctctatttcttcctgacctggtttattgtt
tacctggaaaaagggctgcacctctctatttccaaagcagggatgggtgccatgcttccctatattatggcgatgcttggagtgctctgtggtggtacgttaa
gcgacatgctgctgaaaaaaggcaaatcccgcacgctggcacgaaaattaccggtgatggtaggcctgtgcgtcaccatgattattggcctggtgaa
cttctttgaaaaccagccggttattgcgattgtcattctgtctgttgcgttctttgctaacgccttttcaaacctgggttgggtggtctggagcgatgtgatt
cctcgtaatttcctcggtaccatgggcggatttttaaatatttgcggcaacctttcggggatcgttagcccaattgttattggggttattctccaacgcacgc
aaaacttccagtatgccatgtggtatatcgcaggcgtcgccggactgggcttattagcctacatcttcctggtgggaaaaattgaagtgatcctgcctgaa
aaaaagaatgccggcaccgtggataagaatgccattaatccggcaactgccaacaaataatgaggtcatcatcaaaatggaaaaaataacctgt
aacgcttgtctggcccatgcggaaaaagacgtacgttttgaatcacgtgaaatcgagcatggtgaacatgacgtcgtcgtgaaggtcgcctgcggc
ggcatctgtggctctgatattcactactatcagcatggacgcgcggggatgtctgttctcaaacatccgatggtgattggccatgagtttgttggcgtgatt
aacaaagtgcccgcaggcagcgagttgaaagtgggccagacggttgcggtgaacccgtccagcccctgcaatcagtgtgagatgtgcctttccgg
ccatcaaaacctgtgcggctccatgcgctttatgggcagcgcgcagttcaatcctcacgtgaacggcggcttttctgaatacgtggtggtaaaaccgg
agcaatgcattccctacgatagccgcgtacccgcaaacgtgatggccttttcggagcctctggcggtcgccattcatgcgataaaaaaggcgggcc
agttgaccggcaaacgcgtactggtgattggcgcaggccccattggttgcctgatcctcgcggcggcgcgcagtgccggggcatctgaactggtgg
cgtccgatctcagcccgcgctgcctggaactggcacgtcagatgggggctacggcagtgatggatccgcgcgatgaagaacaggtcgcccattat
cagcagcataaaggctattttgatgtcgtgtttgaagcctccggcgcgccgattgccgtggcatccaccgtcgatttcacccgccctgccggcaccatc
gtccaggtggggatgggcgccagcccggtaagctggccggtatcgccaatgctggttaaagagctcaactgggtcggttccttccgctttatcggtga
gttcatcaccgcggtacgctggctggaagatgggcgcgtcgatcctcgcccccttatcagcgctgagttcccaccccagcaaattgaagatgcgttg
attaccgccacagataaaaatatctctgctaaggtactcattcgtttcgattaacaatgagaagcgcctggccgggcgctttgttcttaaaagtgaattgt
tatagaataaatcacttcagcgacatcttaacggatacccattttgagcataaaatccattcaaaaacagaatgttgtaaatgaaatttatgatcagata
agcggcaaactgctggacggcagttgggcgccgggtagccgtttgccctcagaagcggagctgactacctcttttaacgtcagccgggtcagcgtt
cgcagcgcggtgcagcgttttcgtgacctggggattgtggtgacgcgccagggcagcggcagctacgtgagcgaaaacttcaccccgcagatgtt
gagtaacgatcctcgtccgatcatgcaccttagccgcgaagagtttcacgatatgatgatttttcgtcagactgtggagttcaaatgcgtggagctcgcc
gtcacacacgccaccgatgatgacattcgccagctcgaagaagcactgaacaacatgctgatccataaaggcgattataaaaaatactcggaag
cggactacgagttccatctggcgattgtcagggcatcgcacaacagcgtgttctacaacgtgatgagctcgattaaagacatctattactactatcttga
agagcttaaccgtgcgctgggtattacccttgaaagtgtggaagcccatatcaaggtctatatgtcgataaagaatcgcgatgccagcacagctgttg
aagtgctcaatgaagcgatgtcaagcaatattattgcaatcgaaaaaatcaaatctacagaaacatcagggacaaaataacagttggttacaggct
caacttacagaactatttaacatagctgaatccgaaatagttgccattaacggagtaaagtgagccagtaacaatttactactatgttagtgtgacata
gtcctactttaaaatcctggcagtgcggcctgaaatcagcggagcttcaaaaggccaagcccccccttaacgtacaataattttcgatatccaaactg
ccccctggaagatcaattttgtgctgcagcttttaattttctacgcaatgctgatatgacttaaaacgatgtgaaagatacagagtcagcttattgtggatg
gctggagattgacgaagaagatcttgagtaaagctcgataattcttctgacgttataactaacggatgtcgcgtaagcgagccttcagggccgcttac
gagcgagtatgacagacgtataggggttcttcatgttctcagtttttgaattgtcaattctactagttcaactcaatactcttagcatgggaaaatttcattt
ctccaggcggctcccaaaccgtgattcctggtaagcccgcatcaagccgggcactcaaagaatgaatcggggattctgacgctggcctacaaccgc
caggatattgacacgacgggccttatcattcacttcataaagtacatgctgcccaagtaagctaatacggcgaaccccttcgccatagcgttcatctgt
ttcatacagcctggggtaatctgccagacgatcggttgcagtgatcagacgctctgtcacgttccttgcatcccaaagagatgcattctcggccagata
ttcaaacaacgccgttatatcctcaatcgcacctggtcgccattctatttcataactcattgcgcagttcctgataacgtccctgtagttgctggcgcagttg
cacggaactcattcccggccgcttatcggcgcgagcagcggcaactttcgctcggatctgttcgtcgtattccgcccgtgccagttcttcattagcgag
gcgttgcaatagtgcagacttgtctgtactgccttctggaaaaatacgaggcaggctttcggccagctcgcgcaggaaattggcgttttcgtaatgtgtg
gtgctattcataattgactccccctttcattcaataaagggcaattgcaggttttttgatgccgcacgcatgggtataacgagtcagagtatcgatattcgc
cccggtgatattgtttcacaggcaggcgataaccggatgcataatgctcatttgcctggcaatctgcgtgttgtcaggttttatgcagagtatataaccttt
cagacacagccaccaacatatgcttatgccgcgccagcggcatgtaaggcggcacgctgtggttacatgtgataccggagtaaaaccgccgaag
cccggcgtaagccggtactgattgatagatttcaccttacccatccccggccctgccagaccatacccgttttcagccatgagagagcttctgtgcgcg
gtcggagtggtcccgacgagggtttacccgaagtcggggcgtatctccgcgttagcgggccgtcagggccgcttacgagcgtgtactgagaacttc
cagccagaacactgacagcgatgacggagtatagttacaacattcataattaaaagcgactctgttccggccctttgggccggggggggccgcttt
tcagttatgagggaggggctttgtggctccagttctgcgctggtccggggtttttctggaggttgtttttgtgtgttgtaactaaagtggctccggtcggggc
ccgccgcttgcggtgggaggtgcatatctgtctgtccacaggacaggcagtgaataggttttctttttaaatgaatgtaattaagtagtttaaaggagata
taaacaggtgtttaaaagatacattgcaccctgtaaggctggcggctggcgctttatgacatgaacggttgtaaccttatggggaagtcccttgcagtta
aatgtggataagcaaaattccccgtctgtgaggcgtgttttgtattaaaaacagggggaaccggatgctccagaaggtggatggtgaggttgttttttgt
atgtagtgctgatttattgtgcactgacgggcttcaggc
Nucleic acid sequence of pAPEC-3 plasmid (SEQ ID NO: 3):
atagggaagtcgctgtaatgagtccatttcacggggccaccacctggttttttgatggttgcgtttatttggtacttaggcattcaaaaatacctccactcga
tcacctgtctttcttgaacacatccagcggcgcagttctgtaaaactatcctggatctcacaacgggtacttttcattgtgacagttggtatattgtcgtagc
tggcatatgcaatctcttttttgggtgtgcgcaatatgcctttgtcaatatcaagaatcctgaacgcgctaattcttcttttgtgctcgttaaatagtgttcg
tttagttgctgttatctgcttctgtgcttcctggtaaatgtaggtaagtacatcataagattgttgctgtgtaataaggaaattacggctaacatccgttgtt
gtgagcgttttattatttcttaatccccaataagccacgatgatatagagtggatatcctttcaaatctgccacacattcaggaatgacaggattaattttta
acttacgcgccatattttctcccggttgtttgtctgaaatagaaacatccccggaatacagcgtcataagctaattatgaaaagttgcagccactcactacag
gcctttatctgattgttcatcatcatgcatagctaaacccccttacctggagtggagactggcaaatctggtgtacttctgcgttgcagcctgtacgacttgc
tcagggagagagtgagtgagattaactcaacgcgagttcaccaaataagggggtttaagtatgcagaatgatatttgcggataaagagtcgttacctgtgtag
atggcgcggcacgtcactctgttacgtagcccgttgctgacagatgccagcggtgcttgaaatgcacgaataataacctccttatgccagggctttaggcaat
gaccgctgtccggggttgttaaagagcatgagatgacaagcgttctaacgggcgtcaaagagtgctcattttccaggcaatgagcacaaaaaacgcgatgaac
gagctttttacaatcatctggatagtattgtgtgtgcgacatttaaaacctgtcaatgccacgagagcgatctaatgatctggataacacctaaaaaatagaa
taatattctatttgttaaatgatgtacgatctaaaaatctagataatactgtttgtgtggtttattcgatcgtgtgaatgggtgtatgcagtcaaaaaaactt
ttttgaatatacaaaattctgaatttctgattttgtaaggttttgtatggggaacgtcagtaaagaaggggcttcagtagctgttctgaaaacgattttgaag
cagtcacagatgtgtgctgaatcaaaaaggaaacgagccaggcttgaacatcgttgatcgattgcgcccatgctgctaaatttgcaggcaagttttccgcgat
ttgtaaatgcagaaaacgaggtgctgaaattttgttcgagaacgtaaatttcagataaagaaaacccccgtaatctcgtcttttccagggagcgcagattagc
gggggttgacctcaaggcagagccttgagctgataacattttatttcaggtaatcgcttactacctgaagtggagtttaattccatgagaaggaaatatcaac
tcttacagtgcaagctaagtgcagcttgctgtgattagcaaacgtcactggtctgaactatcaccggaagagcaggtccgtttctggcaggactacgaagcgg
gtattgaaagctcattcctggtcccccaggaaaacaaaggcggaacaacaaaacgtcgccggggtgaacattccactaagccaaagtgtgagaatccagcctg
gttccgtcccgacaactataaggcgctgggcggacagttgggccacgcttacaatcgtctcgttaaaaaagacccggttactggtcagaacacactcagaatg
cacatgtcgttgcatccattttatgtactgaaacgtcagagtgtcggtcgcaaatataaattccgaccggagaaacagcggctgcttgacgcaatatgggttg
tacttgtgagtttttgtgatcgtggattgcacactgtcggcatgtctgtctcccgcctggcagaagaaataagcccaaaagacagtaagggagacgtgattcc
tgaaaccgcagtcacagtatcgcgcctttcccgtctgctggcagagcaggtttcctttggcacgttgggaacgtcagaaaagacgatatgggaccgtgaatcc
cgccagcgactgccgaaatacgtctggatcaccgaaactggctggaaaatgctgggagttgatctggtgaaacttcaggaacagcaacgtaaacgccttgcag
aaagtgaaatccgtctgcaattgattaaagaaggcgttatccgcgagggcgaagaaatctctgttcactcggctcgtaaacgctggtatgcgcagcgttctct
cgatgccatcaagtcccgacgtaagaaagcagcggagcgcaaacgtgctaatcgcctggcaaaactgccgtatgatgaacagcgaaacgaaattgcgcggttt
attctgaagcgtatgccgccggacgaagcgtactggtgcactaaagaaagattagagcaactggtagccagagatctacgtcagcttgaactggccctgacag
cttcgccaccccactagtttagctgcctgatacaatcggcactctcttcagccccttcggggcttttcgtgttgcctgcatttcatgaaattcctcatattca
tgacgaccgtttttcattttgtcccgctctgacagttttttgatggccgcaatagggtatcggagtaaaaacgccttcggttatccacaaaaacccgcaaagg
aatagcttgaaagcagcaaacatagaacatcaaacccgcaatatattcttaacccctgttctttaatcccctgcgttgcttcgccgcagggaaaatatttatc
tttgaaacaactgtggataattacaaaaatgccttcgcttgcagcggctaacgccgcgccgctcagaatataaaagtacctcccaccgcttcgcggcgggcat
atggacttctgcctaatgaaaaacaaagcccctccaaccctcttacaaaaaaacgctgcaaatactcagccacagcgcccaacccatcactgaaaagcgccgc
cgcccccgcccgaagggcgggaacaacatcgctttcaataatggatgttgtaactaagaaattacatggctgtcagtcttctggctggaagtaccaagtacac
gctcgtaagcggtcctggcggcccgctaacgcggagatacgccccgactgcgggtaaacccttgtcgggaccactccgaccgcgcacaaaagctatttcatgg
ctgaaagcgggtatggcttagcaggatggggataggtaaggtgaaacctatcaatcagtaccggcttacgccgggcttcggcggttttactccagtatcatat
gtaacaacggagtgccgccttccatgccgctggcgcggcatcaaaaaagaaagccccccaatgacgggggaaatagcgttttgatcaaaataataatgctagt
gtgcttcttgcgtgccattccaggcacaaactaaatttaacgtttcgttaactacaaagaaatccgatgagttaatcacttgcgaaaaaaaatcatatagtaa
attactatatgattttgcgttgtttcggagaattaaaaaatgcctttgattgaatacatcaacaagttctatcgggggaatcaggcatcgttcgccaggctga
ccggagttcaacctgcgcaggtaacacagtggatcaacaaaggattcatcgtggtgaatcacacactgtatagtccacgccgaaagctgggaatttaattcct
ggtcaatatttgacatcctccacgccctgaaggacggggatataaggcgcactgtatcctgttgtctggcagaaaaaagaaagccccgtagttaatttttcat
taacccacgaggcgatcccaatacttcaacaacaccaggatagacttttaccgccctttacgcaataccatgtattttcaagtttctggtcgatatataatgt
tgttggcgcttgaggctttctgcctcatggcgaaaaggtggtttgtatcttgttgtgcggcagaaagaagaaagccccgtagttaatttttcattaacccacg
aggcgatcccaatgcttgaacaacatcaggatagcctcttaccgcgcgttacgcaaggagaagaaggccatgaaactaccgggaaacgcccttatctggtgcg
tattaatcgtgtgctgcacgttgttaatattcacactcctgacccggaatcgcctgtgcgaaatccggctgaaggacggatacagggaggttacggcaagcat
ggcttacgaatccggcggtaagtagcaacccggaggcgggcgaaagcccgcctttttggggatgatgtggttctggcattaagcgcctttacaaagggggtat
tctgtacggataccccctttttccatgtattttcaggatattaactggtgaatatggtataattagtcaatatgacagcaattattgaggcgagtatgcagcc
caaaaaaacacccgtgattgtggtcaaaaaacgccgcgttctggtcatgccagaaaatcctgtggtgaatgaaaagccgcaggaagtacaaaaatcagcagtt
aatgaaaacaagaaagtacagaagaaagatgctgtagcagagaaaacacgtaaaaagcagcctcagccctggtatttgaaaaaacaaatcacttttcccccaa
aatatccgaaagaatattttgaaaagtgtttcaataaagttcgtgctgtttttcctgaactatggacagacgaaaaaaagaacttgcccctgaaaagcgggat
tctccaggacgttgagaaatacctggcggataacccggatgtggatctgacgattgaagagtggaattgtgcggttcaggtgatgacgttccggtggcaatat
cttcagaattgtactgtaccaggcgcaacacgttacgacctttacgggaaacctgctggtacagtaaaaaaagcacacgcaacttatgcgcaactggttctcg
atgcccgtaagaaagccagcgagaagaaacaattaaaacgtaagggataaagccccgaagggctttatcatttcagcctggctgactgtagaatccagtgatt
ttttccgttttcgtccaggccgtgttcgtaagctgcaagagccgctttcattattgctgtttttgtctggcctgttcgcagtgcttcccgttccagaatatct
tcgtaaacgtccccaagccggacattggtcactttagttgctcccgattttttgcgggtagcttcattgatgaatcgggctgtgtctgcattggtttgttctg
ttttttgtcctggtattaccggagttttcaagtcaagagccattatatcacctttcgtgtgttacattagctttactgtgtaaagttaatgatgtaaagctgt
cgtattacatccattagggattgatgcctaactcttgcgccaaaagctctatttgtgctttagctgtagaaaggctggatgcgcgacgtacatcatgaacgcc
agcgccttcattacaggccgcttcaaaaatatctaaatctgaaatacgtgtacgaagtggctgtatccacaccgggttctcccggagtaacttgtcgagatca
atcgctgcttttctgtggcgtggttttgtcgtgttaattctcgtgaaaagtacccagggttgcaggtctggattgatctgttgagctgtgcgaactttttctg
ttacatgagtcagcgtttctgcttcgaaatctgaagacggcttaacgagagtaagcagaatgtcagctacagttaaggcactgcgaaattccttgctgtcatg
ccctggacagtcaatgatcagcacttcacatatttttttcaggcgcttaatgacttctgatacatcgccatactcttcataaacaggaaccggtaataagcca
ttctgctttcttttttcgttccagctaagaatatcattgtttttatcggtcttcagaatgatgaccgttcgtgaattatttaccagataagaagccaggtttg
ttgctgtggtgctttttcctacaccgcctttatctgatgcgacaagtaatgctctccccatagaaacctcggatatacagtgtaaaggtttatttgtctttac
attgtattcattatagttacgttgtaaagatgtttgtgtctttacaatgtaattgttttttgcgctttacattgtgtgtatgttacagggtaacttttcagga
tgcgattttgcctggtgtgttgttgcgcccgtagctcgctgacgctcacacgcgcagtacagcgccccgccctgctgcaatgcaccaggacggcgtaaactcg
ctggcgctcgcgctaactgcggcattctgcggcgcaaatcagcatgatcatggcctgtcagtccgttgggctttgcagggtgaagggatggccctgtgcatcc
tggtatgttcgtcagcggctgcgccgcgcttcggggcgatgtggccctgctgctccctggtattttctttcgccccttccatgccctgtcgggcatcacatga
atgtaactatatgtatttgatggtgttttttttgaagaatggagggaaaaatacttgataataggtcagttctgacctattataaacacatcgggaggggatg
gtccccaaccaccaaagccgctcagcggcaggagataaaaatgaaaacattaacttttaataacggcactgtttctgttggcgatgtgtttgtatcttcctgg
ggatatgagcaaacgaacgttaatttctatcaggttatttctgttcatggcaaaaaaaccgtaactgttcaggagattcgcgcttcggttcatcggacccatt
caatgagcggatataagaccccattactcaatgatttctgtggtgagccattaaaacggcgagtgcgtgattattacagtacgccagcaattgaaattgagtc
attcgaaacagcatataaagcatcaccggaagaaaaacacgagttcacatcatactactaagaaaatagttgcagggggtatcccctgcaaccatgttaaatt
gttaatagtgaatacaggggcttaatcgcccctttttttatctggtgcgcagtaaaactccgtccttcaaggcgtgcgagaatgccaggcgaatgcagcaatg
taatgttgcgcccgtagctcgctgacgctcaacgcgcagtacagcgccccgccctgctgcaatgcaccaggacggcgtaaactcgctggcgctcgcgctaact
tgcggcattctgcggcgcaaatcagcattatcatggcctgttggtctgtcctggctttgcagggcgaagggatggccctgtgcatcctggatgttcgtcagcg
gctgcgccgcgcttcggggcgatgtggccccgctgctccctggtattttctttcgccccttctatgccctgccgggcattgcatgaatgtaaatatttgtatt
taacgatatttattttaaagaaaggaggaaaaaatacttgataataggtcagttctgacctattataaacacatcgagaggggatggtccccaatcaccaaag
ccgctcagcggcaggagcttaaaaatggaaaaccacgccaaatttgttgctactgaaattcttaatcagcttggtggaaaccgctttattgctatgacaggcg
caaagaactttgcctgttttgatgaaaatggtgagtctggtctttgctttcgtttaccttcaaattttgcaatgaagggaattaatttggttaaaatcaaatt
aaccttctctgatacatacctggtgacattttcccgtgtccgtggagctacagtaaaagaaatatcaaaatttgataatatttattgtgatcagcttgaatgt
ttatttaatgaacaaaccggacttgcaaccagattgtaatttttaatatggggggattccccccatatcaggaggataaatgaaagttcttattggtaatata
aatattgataattatcatatgctgtctgcattagctgggattgcgggatttgacaggagcattgagtttacatgtgaaatatcagcaagtatcgaaattatgg
aggatgatttcgtaaataaagctggtatattaaagatgttagatgaatttattgaaaatgatttttcaataaagctcgtctagaaaatacttgataataggtc
atttgtgacctattattaatgcattgagaggggatggtccccaatcgccaaagccgctcagcggcaggagttaaacatgaaacaatctacttttcctgctatc
gtctctacaactggtcatgtgttttctgttgtgcgcgtgacgctttgcacaatttgccttaaacatgaaaaaacaggcgaggcttatgtcgtcatttttacag
actgccataatatccgtgattataaaaaaggcgttgttccggttcttggggagctttatcaggaagatgtcgatttgattaccggaaaaagttaaaagaaagg
ggctatatgcccctttactgttaaaaggggagactatctcaatatgaagataagaaaaggtgacagacaatattatctgaataaagaaggtgatacgtttcat
ctggtgaagcgagtaaaaactttttctaagtctgcgactttaggaaaaacgaaagcaacggtgaaaactgttgccgatcttgtttttcatgagaaggcattcg
acactatcgactttgccagcgacggattaagagagaatgataaagaaattgtttctatgatgattcaggaaatgagtgagggaaaaaatgccaaatgaaaaca
tcaccagaaaacattaaacgtctgcggcaaaaaattggcctgacacagaaagaagtgccgaaatatttagcatgagtccgcgtacctggcgtcgcaaagaaga
acctgtcggaactgcaagcggtacagcacttacaccagtagagtttaaatttcttctgcttttagctggagaacaccctgattatgtactttgtaaaaggaat
aaaaagaattctgattctggaagtaattaaggaatattaagatgaaaatagaattaacgtatgacgaaatcacggaataagctacaaaaaccataactgaatt
tatggctggacttaatagccccagttgcattgaacaatgagcaattgcgcccgtagctcgctgacgctcacacgcgcagtacagcgccccgccctgctgcaat
gcaccaggacggcgtaaactcgctggcgctcgcgctaactgcggcattctgcggcgcaaatcagcatgatcatggcctgttggtctgttctggctttgcaggg
cgaagggatggccctgtgcatcctggtatgttcgtcagcggctgcgccgcgcttcggggcgatgtggccccgctgctccctggtattttctttcgccccttcc
atgccctgtcgggcattacatgaatgtaactgtctgtatttgatggcgtttttttttgaggaatggagggaaaaatacttgataataggtcagttctgaccta
ttataaacacattgggaggggatggtccccaaccaccaaagccgctcagcggcaggagcatgaaaatgcatttcacaacttttctaaaaaaacatttcgatat
agaaaaagtcgttggtacttctgattctggaaatgataccgaatctatttatgtttatgaaaaaggaaatgattgcgaaccgttgtttattctgcatgaatcc
tggctaaatgcagaaattaaaaagtgtggggtatggactattggcaatatatattccacattggaacacggaaaagaatattctgaacaggaactaataaaaa
tgattaaggagggtaaagtaatcagcaaatactaatttggggttggtaacaggctgtggggcagcctgttaccaaatgacagatgcatctttggagattctca
aatgtcagtatgtgaaatgatagagcttgcaaaccgcattgtaaatggggaacctgtaaaaacacccgcgatagaagcatggattattagcgaaatcatttgg
tgggcgcataaccgggataatgctttagtttgagtcatatatggggagagtgtcatgctctccctttcttattgggtagcgcgttgtattacatagttagctt
tacgttgtaaagattttattgtctttacagtgtggtgcgttttccggcggttgcgctgcgcttaggggtatgtggcctcgctgctccctggtattttctttca
cccttctatctgataacttttcttgaaagaggctggggttttatgaaacatttaaaatataaaggatatttaggtacagttgagccggattttgaaaataata
tcctgcatggaaagctggcatttattcgagatctggtgacttttgaggcagaaacattagctgacctggaacgggaatttaaaacatcggttgatttgtattt
acagtcctgtgtggaggacggaaaggagcctgacgcgccctttaaaggtgtgtttaacgtcaggcttgatccagagctaggcactggaaaaagaagtcattaa
tcatcgcgcaggggcttaatcgcccctttttgttgctcgctggcgttggtgttttctggtattacgatgtagtgtgttgtatttacaagtaatgctttacgtt
gtattacgatgtatactctttaattgcttgcaatctcttttcacgttcgttgctttgttcagcctttttcagttcactgaatatgtactcttcatctttaccc
gtgagttttgaaccggagagcaagcgattaacaactttacgtcgaattgtggggtaatctttggtttgagcgagtatggacaatatgcggtcactgctaagaa
tctggcctttccctgattctacataatgtagaaaaaatgcgattgctgacacgcccaaaagaattgtaaggacggctacaccatcaagacgaaacgttgatat
aaaggaatgcgcgaccactccaataattgccaagcagagacataaataaatgaagacgtttttgttgcgttgttttttttgtgcgcgatcatgatcatctatg
attgccagtatcttttctggggttggttgagccattttttttccttatcagggacacggagtgtttaattacatagctttacattgtaaagctatgtttgtct
ttacgatgtaaatgatgagatgtgcagttaaaagtgtcaatcaggtatcatatagtaaaacactatatgattttctatgtatcacatgcatagcgataaaact
ggataaatgatttacactgtaaagattgaactgtctttacagtgtgtttcataagtcatacgaatgaaataatttgcttttgtgtgtatgcgtgtgtataatt
gttttcaggttagggaggacatatgaaatctgctgacctgttgaaagaactgatagctgctggttgcgagttgaaacggcataaggcaagcagtcaccagata
tggtggtcgccaataacaggaaaaacttttcctgttcctcatcctaaaaaggacttgccgctaggtactgtcagatcgatcaaaaaaatggggggatttaatc
cccgccgactttggaggtagccatgttcttttctgttggtgtcgaaacaccgaaagatgatcatacagcatacggtatcaccgttccggtttttgattgcttt
gattttggatgtgtttctgctgctgattctcaggcagaaatacccgcaatggcgcgtgaagcgatattggcaatcgtggaagagatggtaataagcggtgctc
attctgttgatgatattcatgatgaagggtgtttgacttattccgccaacccaaattataaccactgtgatagctggtttgtaattgatgttgatttgtcaga
aattgagggcaaacaacaacgtattaatatttcattgcctgatgtattgattcgtcgtattgatggatatgtgagagaaagtggtggcgtatacaaagatcgt
agccattttctggctcaggcggcacgtcatgaacttgcatataaataaaactatccccgcgttgtgcggggattttttttgatagctaaccgctcgccgcagt
ccagcgaccgagcatagcgagcgagcgacgaggaagcggaagatccaccgttgccaggagaaaagcaggtacgcactatgcgttctgccctagtgatgctttc
acaaaccacgcagcctgaactattacgccatgaatccagcgcaaatcggcatcacgattttcttttttcattttttcgccacttaaagccttgcttacgtgac
tttttaccagattttcgtgtaattcttttgcttcttcaattgtgaaaccaccaggcaaatttggcgcttctgcttccagcgatgccagtgcgatttcataagc
cccgacgctcaatattgtctcggacgtccaggctgcctatacgctctttgatttctttaattagttctttgtcctcgtggtaaaagtggtcattagggactcc
ttttcatatttatggttttcttgaatcaatttcgtctagtttgagttttatattttgctggtctgattttggtaattttctcatgctctcttcagttattttt
ttcatatcaagaaatgctcttgcatagaagcgcagacttgcctctttttcatgttgtgcaacataccaggttgcgccagaaaatgccgcagacaggcataatg
tgcacagggcacaaattgcaatggctgttttgatactgaatggttttgctgatttagttagtatctctagttgttttttatggtcggaagcaatttggtcaaa
attttgtttgattccagaaaaagtatttattgcttgtttggttatctcgctctgacactcattagatatttgtcttatatcgctttttgcaatattaatatct
ttattaatatcaatgctgctttgtattattttatttactttgtctgacagaacggcatccagacgagcaggtgtctcgtctatagcgttgataattaagtttg
ctgttttttcgatctgtttttctaaagcaatataatcttcaagaagatcgtttattttttgatctttaaactcttcagagatactacgagacattgtttttct
ccaattttatgaccgggtacatcccggtcatattgtatcagacgagattgaggtttttaaaggcaacatcaagatcgctgttgaatccattcatcaacatttt
tactgctcttaaatttgacaaatcttgccgctcttcctttgaagaagctgaacgaattaatgaacgaaaatcacgattgtcattatagacatcatcatactgt
aggccgcttttatttaaaatattgaaaatgttgttatggtaaacaacaacaggattttcacctacaatttctttacatacgtcgtcagctaagaaaactgaat
attgtttgttaataggggtgtccttttcagcaagattgaaaattactttaaaacgatcctgttcaacacccatagccattagtgttgtaatggtttgaattga
gtctatttgttgttttgcttctcgcacaacaggaacgatgaaataatcaataaggtcatgacttccctgatattcattcatctgtacaaggaattgctcaata
tttgatgagcctacgtcaatgatggtgcattcactatcatcaatacgtttgataatttcgtcaaactcctttgctgataatttctcgtcatttgtaccatcag
tgtttaatgtttctacttttataatttctgacccctccagacgaggtttcaataaagtttggcaaatggtcgatttccctacgtttccgctattatttacaat
tgctagttttaagtacatttttcttaccttttatttcgtatataatttccaagagtattgctaacttgtacaaagttagcacaattccaagattttattgttt
ctatagatacattgttatctatagcttgttgagctatctttgcattgttgaagcaaactggcaaatatgcattaagtatatcattatcttgcttcgtttttct
gtttttgtcctcaatggttattgctttttgatacgttttctctttatttgaaagaagatgagaaacgtcttttttctcggccctttcttttcgtatgcgatgt
aatatgttttcaaacgtctttaacgttagatcaaaaccttcatcattaagagcattaacaatatcaatgtttcttatgccttgttgctgcgcttcttctattt
cagcaaaacgctctcttattcttccagttttacttcgcttgaccttctgagctaagagcgaaccaagtagtttattttctccattgttcactttgtcagtctc
caggacagtgaaatcatgatgagcaattctatcatgaacgtcaagcaatttttgtatgtatatgctatgcgtttgctatgtatatgctatgtcattggttttt
atggtgtgtttttgctatgtattagctatgtttatggtgtgcatttgatgtgtgaatgctatggaatcaaataattttctaaaacacaggcacgataagcttt
gtacacaagcccgttccgggcatgttacaatgcttgtgccaaaggggacgcccctttgcaaccccgaacggcgcagccgttctagtgcggttctacgaacctt
gaggacttatgaagaaaaaagccaacaaaagtgttcatgtgacattcagacttacagaagaagagtatgctccatttgacagagcaatcagagagcttgaaat
tagtaagtctgaatttttcaggctgcttactattggtaaaataaaaaactacacatcagataaacgccatatcccggaatacaaacggtgtctttctcagtta
agttgggctgggaataatattaaccagatagctcaccgcttaaactcagatcatttaaagggtattatatcagaggcgctttataaaaagattttaaatgtgc
tgattggtattcgtgatcgccttcaggagcatagccaaatgattgtcagatatggtggtggtaatgacggtattgttgattacctgataaatggtcgcaaagc
agaagtcagtacacacgtgatgaacttgatcatcgtgttgttcttgatggtgatctccagactacagataagattatcaattctattgaagacaacaaacagg
accgttatttgcacattacgttgtcttttcatgaaagtcacgtatcaaatgaaatgttaaaggcggttgtggatgattataaaaaattattgatgaatgctta
tcatcctgaagaatattctttctatgctgaagcacatttgccaaaaatccggcatattcaggataatagtacaggagagttaattgaaagaaagcctcatatt
catattgtcattccaaaggttaatctagttacaggagataaacttgatcctgttggccttatagggaaaggtcatactatcgaacagctagatgcaatacagg
agcatattaacaataaatataatcttgccagcccgaaggactttccgagaaaaaatagcgattatagtgttattttaaagagagtaaaagcagatcttgatgt
agagcgtaattctgcatttaagaatgaattgaattatcgaattgaaaaagaaaaagtaagtgattatgctgtattcaaacagattgttgcagaatatggtgag
gttaggataagaaatgaaggaaaggccagtgaatatattgctgttaagttaccgggagataaaaagttcactaatctgaaaagcccgttgtttcgtaaaaact
atattgaagagggaaagttgactcttcaaaaacctacgcctaagcaaatagaaaaacgtctcaatacctgggtaaacaaaaccagtcacgagattaaacatgt
ttttctcaacgccagcgttcgtaaggactatacaagtttatctgaagcagagcagattgaatttctgaaagagaggataaaagaatatgactcaagagaaaaa
cttaacgaaagaaattctcagcaaacgtcaggacgagcgggaggttacaagtcgtgtcctaaaaagtttgccagaatccgtcaatctgaagcaacagtcggat
tgtcacgtatgccccaacgcggtatggtttacggaatcaatggattcacaagacccgactctatcagtgtattgtcagatatttcgcagcgtgatctggcaga
gcaattaccgcaaagagaacatcctggtcaggatgtgcgacgggattatgatagacaattcacagagtcaggaataaaaagtcttgaacgctcttcttttttg
tgtgaaacgatgtttcagactctgaatgaggcggcagagaaaaacgaaattacaacgatggcagaaattcgcagaaatattgatcctgtcaggtttctgtctt
ctgccgccgagcgttttaatattattccagcgcagcataaaatcagaacagcaaaggacggctccccccggttttctgtggggaatcgtaatatgaacgcatc
tgattttctgacaaagcatattaatcttgcctggaaagatgcgaaatcctttctgcttgaagtttattcacagcagttagaaaacacgccatatacgcgctat
ccaacctacagacgccttacgcatcatgaagcccgtgagcgtcttaactcactgaatttatcggaaaaaacattacgaaatactatcaggtttgagcgtggca
agctgtataatgatctgcgagagatgagacgcgagttgaagttaataccacgtgaacagcgtgatattgccgtgggtgtgattgtttacaaaaaactaaccac
acttgagcgtctttctgaactcgacacagaggggcgacacataattcgtcaatatcatgctgactggcataaggataaagatgaaatgaaagcccttgaacgt
ctcaaaagctatctcaacttcgatgaaatcaacgccatttctgctgacgaacccgaactttcgcttcagaaagcggtggattcccagcgccgcttagaagagg
cgaaaaaggttaactccaaactaaaagatcttgtgatggataagcaggattccaggattgtttatcgcgatcaggaatcggaaaagcctgtctttaccgacaa
agggaactttgtcgttgcgggtaaaaatccctcaaaagaagagatcggaataatgcttgagtattccagggaaaagtttggtggtgtactcaaacttaccggc
tctgaagatttcaaaaaaatgtgcgctgaagttgccgcagagcaggacatgaagattattttacgcccggagcagtatcaacagatgatgctggaattaaaag
cagaacttcagggtaataagtttgagcaggtggaaacacaggaaaatagccaggagtccgaatccagaatagagaaaggtgatgcattgaaggaacaggccac
cgaacaggaacaggctaccgaacaggcacaggccacagagcaggcacaggccacagagcaggcacaggccacagagcaggcacaggccacagagcaggcacag
gccacagagcaggcacaggtcgcagagcaggcacaggctacagagcaggcacaggctacagagcaggcacaggctacagagcaggcacaggccacatcttcat
atgatcctggtgttattaccagggcaaacaccctggattcacagatgcttagtaaaggcacaaatggtgaatttggctatttgaaatctctggatagtgatga
aaatgagatttgggaagtgctggggcatgtacctggagacagcgatgatatttttgacgttgccagttttgataatgaaaacgacgcaaaagaattctgcaaa
attgttaatgagttaggtattgacagaacacaggctttgattcatgagcagctttcttctcaacaggagcaggctgctgtgcaggagacgtaccaggaatcac
cagaagagatactcgccagtatttcagctaacgagcatatgatctccgggcttgaaaacttcctggttaaagaccgttctcagttcagttcctgcaatgggga
tatcgttgtggaggcggaaattaccaggggggaaggtggtctttatcaccttgcggtcgctggtaagcatggcttagagcgtggtgatgcagttgccagagtg
gatgttacggagcagcaattcgccgcgatcactggaaaaacaccttctgaagttttaacgggggaccaaacatcggctcgtgttccggtaattacaggtatcc
atttcagtacaagggctattgaaaatgtgaataaactggaacaacagaaggattatgtctatttctcaacacatgaaggcctgaatgctgaaataaaagattt
cagctctctcaaggatgctattgagtggggccgggttgaatgtgagctacatgatctcaataagcgcgatactgtcatttatcgcgtcgagtctgaacatatt
tctcaggggattgacgctgtaatgaaaaatgctgaacgcgtggagcgtcatgaaattgagaaagcacagggacgggattgcacaccggaagacggcaagattc
ttgaggcaattgatcgctttgaggacaagttcagaggggaaggactgaagttcgaaagggagaaggccgaatccgatttgcttaatcacggattcactcgtga
aatggcagaagatgctcttggaaaacaatttgtccaggccagagaagatcatcaggaatcgcagcagcagcgtgacgattcacaggacatgcactaattaaga
aaaaagcccggttttcgggctttttaagaaaaaaagtcaaccccaaacagaaatagcagctacaaaaatggacagtaataatgcaataaaagtaatgttgagc
catatgttggacgagtcttttaagactaatatggctgttaatgccattgtaatgaaactaatggtaataatggctgcaaataaatattgtacaatgtttttta
ttgtacctggttgatccccaattctttctctgtaatttgcatcaatattagatatccattgaagtaatagagcaataaaacagcctactccgcaaaaggtgaa
ttgcacagacgtcgggtttgaatcaaacaacgagatcgctaagttatataagtataccgcgcctgaaatcaccccgacaactaaaatagagatgagtataatc
cgaagaactaatgtattgctttcctttaaggaatatattaaagccgctttttcgtattctttcatgatttaatccagtctataagtataagaaccaacaaaac
tgtccaggattcattttctgtgagcatatgaaatcccatccagagtgatgatactggcagaaacacgcccacagccggaattattagtctggcggagaatatc
cttatcaggagtgtcaatagtgataagaacagaaaaagagtgatcatgtggaaaaacattcacatcacctataacactattaagatagcctgccagatatcca
tttcagacaggctatatacagggttcaattcattctatgaaaggtgtatagggattgctaatgtttgtgtcgatcaaaaatctaatattaatagatgagaact
aatatacatatttccagacggatggggggattttatcgtagagtttattcattgtgagttctgcaagcctgtgatcggcggcggcattaaacgactcaagctc
agaaggagggaggctgttttggttttttcaataatcttttcaagtgtgtcgagtgttgagcaacgtcttaactgaaagagccagtcttgttttgttttcattg
tacaattcctgaagaacatccttgtcctatagtagctacaacgatgtttgatttctactgctggatttatttcttacttttttgtgtaaactcaaatcccagt
ttcccggttgctttgtcaaccagttttacttttgctgaaaattctttgtttgttttttgactaatgaaacctttcaattcacttgtaactccttttgttatta
acgtttcaatttgtttatcggttaatgatttgccgcagaaatctttccatatcttaaattcacatcctgtacaggaatagctttttggcctgataacaatctg
tttaccacaggaaggacaaggagaggacaggcgttgtgactgaccagacggcgcgctgggggaaacttttatttcagcactgttcgcattactgatcatcggt
atcagatcattgtacagttcgtcaacgaactgttcgatggtcatttctccgttttcaatgagtgtctgtttttccgcccataatgccgtcatatctggattaa
cagctatatccggcagagcatcaataagcgcatacccggtatctgtcggaataagttttcctttttccagggtgatatagttccgttttttcagtgtttccaa
tatggaagcccgcgtagctggcgtaccaataccgccatgttcgtcttttttatcccgatctttatccttcagtaactttttaatcactggatcagtgacaaaa
tctgcgacacgaacaagcgcggcaagcaaagtggcttcggtgaataatggcggtggcgttgttttcttctcattcacaaccacttcttttgtcgtcactgttt
ctcctgtgcgaattttacagagaagatcaaaagcggaatcatcattttcgccttctgcttcatcgtccccggcattttccacaccaagaaatgcttcaaatcc
gctgtctgtagttttcctggcacgggcataaaacgactcatcaccacactgaatggcgacggatacttcctgatatgccttttcaggcatgaactggacaaga
tagtgttgtgcaattgccaggtaaacgttgcgttcgtcggtgctcagtgcgtttacgtccggtacgctaacggttgggatgatggctgtatgcgctgttactt
ttgcgctgttaaatgccttgcttttacgtgtcgtatcaatatccattggctgatcaaacaccgacttcagggcatcgattatttgtggtgcttcactgaattg
ttcgtctgaaagatatgaacaatctgaacggttgtaagtaattgctttgtatttttcgcgtagctgctgtgtgatatccagcgttttttgagcagtcattttg
aattttttattcatgtactgctgaagtcgtaccaggttaaagggcaacggtgcagccgtttttttatcatctgttgctgccgcttcaactgtggctggttttc
ctgcaagtgatgttgctgttccattcgcccaggttttatcaagcaatttgcggtctgtcagcggtgcgaactcaccaggtttccagttcgccctgataacatc
agcgccgcgttgaaaatgtccggtcatggtgtagtagaagctggatttatgatttttattggcacgggtgcgatttacaatcaggccaagcacgggtgtctgg
acccgtccgacagacaaaacacctttatagcctttagtttttgccgggattgtgtaagcacgcgtcatggataaaccgtatatcgcgtcggctacagaacgcg
ccagagccttgagatagagtcctctgaagtcacggttatttttgggatttgccagtgcctttttcactgccggaagggtgttgtcgttgatcagaacgcgctt
aacgggttttgtgttgcctgcatattccaggacctcatcgacaagtagctgtccttcgtcatctggatcgccagcatgaataatttcggtcacatcggcacgc
ctgataagctcaataatcgttttaacttgttttgcggcgctttctacaggctggtattttacgggataaagacgcaaagggagtgtttcaatcttccactctt
tgtattcaggattatagttttcggggggctgtgattcgataatatgaccaaaacagttagtcacaatagtattatcactttcaaaccagccatcatgacgggt
aaaattgccaccaagcgccttaacaatatcattcgctactgcgggtttttctgcgataaaaagtctcattttattatcctttaaataactaatgtactttcaa
tagatggttgtcttttgaataaagagaagtcggtgtttaatcgttcaggctgaatagtgtgaagtaaaaggctttcttctctggatagaaggaaatcacccca
ttcgaactcttcccacacatcatgactgttttcaccaggcttatttaataaggactccattttgggctggattatggctggaggcttattttcataagcggaa
tgcaccctgtcactccatttacaaaaagcaccacttaaaagaatttcctgtgacaccatatcaaactcattatcttctatataagaattaacctgatgataaa
ttaaccccatatcttcaatattccaggtgtatttggtagtgaaccatcttagctttttatctgtatgcaaaagaagtccattaactgcctttctgcctttccg
cattccaacccatacacatttattaggccacggcctccctacagacaatgattcgccatcaattattaaatcaagttcaggtatggagatggatttaagagtg
catttattcgccatgcgatgaaaaagtttagggacaatatgtaggtacatatctatttcctttatctttatcgtagtcaaagaagttctgatttgttgacctc
atggtaaacaagttcaccattgcgtattaaagataagtctttaatattgcgcaaaaataaaaagacagactcattagtatcattaataaaaaaatgataaaaa
actcaaccatttcaggtatattaaattttctgtgtacagttcttaataaaaattgacccgttgccacccagaaggcaaagaaacaaaccacgaacactgaatt
atcgagtaaatcgtataacgaatacatccagtgaccagcaggatatggcaaaccaaaagaagaatgcatttctttcccccattatgattttgaagtattcagc
attagtattcatcttcataaggtagccaatcgtaatcgtttgagttccagtcaattggaagaaatggctctgtttgtttatggcaggagaggcaaacataaat
atttttctctttatgaaaaatcatcgacttacatttatcacagtcgcaccaaccctcgactggttcagggcattcaggtaattctttctcctgatgtttacca
tatttttcccattcctcggcatcctgataacctccccatactgcttgtgtaaaactatcgatatagtcacctgactcatttttatcaaaattaaatttatata
cttcgccatttagttcaaatgacatatcaaggtcaggtagtaattgcttttcgaagcgttgttgtaactctttcttttctttgggtgataaagccataaaaac
ctctatcagttagttatgtttgtatcgccactcccacggtggaaccggatttgtatcggcccacaaaccgcgtttttgttcctgcgcttcccgttgcagagcc
ggtaatgactcatccacgttgtaacgtccatatacccatgcagcgccagactgaaccataaagcggctggcatccgtgccgttcgtcgtgaacacatgaccga
taatgcgtccgtagcggtctttatccagtgtgccgtaaaaccccgtccttcagggcgaggatataaggcgaggttttccacctggctttttttgtttaaaatg
gtcgaatgaaacgactacaggcatttaaattccagttaagacccggtggtcaacaggagcgagaaatgaggcgcttcgcaggcgcatgtcgtttcgttttcaa
tcgtgctctggcgcttcagaatgagaaccatgaggcagggaacaaatatattccttacccaaggatggcctcgtggctggttgagtggaaaaatgccactgaa
acccaatggcttaaagatgccccatcacagccattgcagcagtcactgaaagaccttgagcgggcctacaaaaatttcttccagaagcgggcggcttttcccc
gattcaaaaagcggggacagaatgatgcattccgctacccgcagggcgttaagctcgatcaggaaaacagccgtatttttctgccgaaacttggctggatgcg
ctaccggaatagccgtcaggtcacggggttgtgaaaaatgtcactgtcagtcagtcctgcggtaagtggtacatcagtattcagacagaaagtgaagtatcaa
ctccggttcacccttcagcatcaatggtcgggctggatgctggcgtggctaaactcgccacgctgtcagatggcactatttttgatcctgtaaacagttttca
gaaaaaccagaagacgctggcgagacttcagcgaagttaagccgcaaggtcaaattcagcaacaactcggcagaagcagaaacgcaaaatacagcgactgcat
tcctgtatcgcaaatatccgcagggactaccttcacaaagtcacaacgaccgtcagcaaaaaccacgcaatgatagtcattgaggatttgaaggtcagcaaca
tgtcaaagtcagcagcgggtacggtcagccagccggggcgcaatgtccgggcaaaatcaggtttaaaccgttcgatactggatcagggctggtatgaaatgcg
ccgccagcttgcgtacaagcagctctggcgtggcggtcaggtgcttgctgttccgccagcgtacacaagccagcgttgcacgtgctgtggtcatacagcgaaa
gaaaatcgcctgttacaaagtcaattcagatgccaggtatgtggatatacagcgaacgccgatgtaaatggcgctcgtaacattttagcggcggggcacgccg
ttcttgcctgtggagagatggtgcagtcaggccgctcgttgaagcaggaacccaccgaaatgattcaggcgacagcctgaacgtcgtaggaatccacgtcctt
tagggcgtggaggatgtcaacaatcggactcggatcggaacttcatacacaacaatttttgcgggtagtgttttaatttctatggtgtcgccatcgagaacgc
gaataactttcccctgaagaattttctggttaaatgtataaggtattccacctgcatttgcagactgaaaagcggtaagcagataaataaatattgttaataa
gattttcataaaagattaacgttaatttgtctgtgcaatggatagagcgttattcttattttcttcctgccgcttcatcgaaaaatgatgattttccttctgc
tgcggcatcatactgaaatgaatatccaaaatcatcgtcatgctcaaaacctgtattttctccattttttagtctggaaagaataatctcaagttgactgatt
atatcttccctttcactatgacgagtgccttgtaagagaatagttacttgttgcaaaggatatgcatgttcaatccatgcctggctaggtccagagtgcgttg
tatataaatttttttgtgccgaaatattattcagaaaacagtttaagtcatcattggaataaatttctttaatattgttgatgttggatgttagttttacaag
ttggaatggtggagaatttttgtcttcattgttgtctcctttatcgtattctggaaacacaattaacaaaggtgcaccattagttctcttcataaaacgggtt
actagccgcccaatatatttattggacatatgctgttcaatgtccaggacagcatattcctccgctgggaacattttgtttttttcgatggatttaatttttc
tggtaagatgaatgtttctgataagcaaacctactatcaggaagtaaataaatatccagaataaaaaatcactctgtagttgttctatcataagtgaatcctc
taatttgttaatggacaaatcattacccgttcatcaatgtttgtaggaagttcgccaggagtagggacgcgttctcctcgctgggcggcttgtgccagtgttc
gtagcagcaacacgctggcatgaaatatggcctgttcttttgtggctgcgcttgtttccaggtcgaaatcactcagagtcacaacgacttgcccgtttctgtt
ctcgaccgttgccgggtagggaacaaaatagggagcgattttgctggtttgcttgcgtcgggcatcgagtttactgacaagttgataagcccgaaggtgtgtg
tatcgtttcaacatgttcagggagcgatggcctgaaatagcggcaacctcaatgacgttcaatgtaccaagttcgaaaaatcggctgatagcctcatgacgga
ggtcatgaaaatgcaagttttctatcttcagttcctgaagtgctgttcgccaggcacttttgaatcccgaagaggtataagaaaaaatattaccgttaagctg
tgtcggcagcatttgcaggtaattacgggctttcctggatagcggaacatcgcgtggagcaccgtttttggttgttggaagatgagctaccccatgttgcaga
tcgacgtgttcccaacgcaatgacaagatttcaccttgtcgcattgctgtttccagcgccagatgaaaaatgacgtaaagagcctgatttttttctttgaaat
accgcgaaagtcggcgttcttctcctgaagtcaggcgacgatcgcgcccgctactgattttgggttttctgaccagttcgacaggattcatgcggcatgttcc
ccattccacacgggcgatattgaataatgatgacaacagggcaagttcaagacggacggtgtttccggttatttgacgccccgttcgtgggttaatttgcgcc
aaccgttgatcccgataattggcaatatcaacagtggtaatgtcatccatatagcgttcagcaatagggtgacgtttaatcacatttacgcgataaaactcct
gtagatggccttttttgtggatggatactgtcttcaggtatttgtccagcgcccggctgattgtcattttgcgaattttgattttctgaaacatatcccctcc
agacaaaaaaggggggaaggtagagattcttgcttcgaaatgcaatatcaccgtgccaatccggtgtgtggagaacttccggttcctctaacggtagttacag
taatcttggatcacaccgaggatcatttactggtcgcaatacgggaagtggtacgctgtttgtttacgcctctggagggaatggtggttccgccgggggagat
tgtgctaacacatccagattgcagggatacgttgcaggggcattgattagcacaaatgcgagtaataacccatcttatgggaagacagcattcatctcttttg
ctgtccctgctggcgcaacttatcagatcacctcttatccggcccaaaactactcttgtggctcaggggtattttcagtatttggataccaaacttaattgag
agttacacaggacgcaatacaacctgtcttgtgctcgtatttgtaccattttcgtgaaccagatggactctcgactacctggcagtaatgagaatcctcagca
tttcccattctggcaatggcacaaaagtcaaattgccccaagtaaccagtgctggaaagttgagtaaccttgagctttccacctaatgccctccacacaccgg
attggcacggaggccagtgccaatccggtacgtggcagaaaaatggcggcggtacagtacagatggttactgcgacagcaagtgactggaatcagccaattgc
caccgcgacttgcccttcaggaaagaaagttacaggaggtggtggaatgtgcagcttttcaaatgccattctcaagcgttcttccccctccggtaacagtgca
tgggtagctggttgcagccaaaacaccaaccagaatcagtattattcagtcactacttatgcattgtgtcagtgagtattctggtatgaattagcggaagcag
tgaacagtgatgtagtagctgccatataacgtaccgttatacgcactccagttcatctcgccgtttgcatctggattgtctgtaggccagacacgcgtcatcg
cggtttcctttccatctatgcgatatgtattaacgcaaagtttgaattttcctagagccttcgttgtggttgggtacaaggtgaaggatgttactgatgtcca
cggcgcagatgaacctattgatttccacgaaccggattggcacccctatttctatgattaatcaatacatactgcatcacaacgataactagctattgaaggt
gggtaagctgttagttgccagtttgatccggacttagataacgagcattctgaataatcgtttgaattatggaaacccgttgctcttacatgggataatgcgc
aaaaagtatgctgccccaagtttgactgagagtatgggaaattaagctgataggaggagactttccctatgcctcctccttttttccacacaccggattggca
ctggcctccgtgccaatccggtacgtggagaagggcatcaggtagcactgttctgacaggaaaaatagcaaatggacaacagatcccattgccatctggtttt
tctgccagccagtgtacctggagtgtcagtaatgcagaaaatccgcacgggtggaagccgaattattttgcggggtcagttgcaacctatgatgccaatcgaa
ttgtgaaatgcggcttctatgacgaatacaacttttatggcggtactcaccgaactgacctgtcggggaaatgtagttacatcgtggtttgccagtgataagt
atcgtcactggcaaatggcgtaaactgtaattgaaccattttgatcttcggtcgtgtcgcaactggcagtccatgcattgttgcctgaaggcatggaacgtgt
gagccatataaatccggtatttgatctgcattgaccgcctccaccaatgactttcttgccagatggacatgttgccgttgctccaggccaacgccacgcagta
gctgaagcggtaacaatttggctatcacctgcgccaatttttttccacgtaccggattggcacgaaagtatcgcccctgtactgtcgcggcctacaagcccgt
taggggagcaggaagcgccagccgttgctgttttttcaagctgtaaatactcgcctgtatagaggcgtccatcagcgcgtacagttccaccttttacctggcc
gccagtgtaaatccctttattattcacgctgcgcaaccaggatgaatcggacatataccagccgccaccataggtgctgttcatccagcctttgctgtttttt
gtgaccagccagccattattcgaagtgatatcaccgttggttgtggctgtatttcctgaaattgcgccagagaacgtaccgttacgtgcgctgatatcgccgg
agagcgcggcggcgttgccgtttaaagtgccgacgttgttcagattgttgctgttcatatcaatcgccgtatgcatcctgttaaggtcgggacgattagttat
ggagtaacgatacaaacggtcgctttcctgcaaatcagcgccaacaagtgccattgcaatatgcccggtcgcgcagttcagcccatagtttgaaggcgtgtca
gaccagccgccacccgcgccaatcgcctgcttgctgctgttgatgtagccgccaagcccttcaatcatgcttgctaccgagcgcattccggcttctgacaggg
actgaccgccattactacaggtcagggcttccagacgggatgttttgctgttctttgcaataccggtaatgtagctttgtccaaaattggattcagaaaaacc
agatttcagatagcctttctgaatgagtgttgctggcgtgattgttttgggtaaagagccagcaagtagtgttgatgcattgtcagcaacataattacgaaca
gcggtgttataggtatttgcgtgttctgctgcattctgccagcgaacattattcagatggttttcaatgttttcataaactttagggataaccatcaccatta
tgccgatgacaagtaatacctcaaggagagatacacctttatctgtctttttcattgctcttaatcctcagaataatattcccggtacgagtgagtaaacaaa
cattccgccacaaaggaacggaccgaatgggatatattttttttgcttgtttaatccgccaagagtgaagaaaataattccgccgataacggcaataaataca
attgaacaggcaatctcaggcgttggaaaccatacgccagttcctgcaataagtagcacatctcccatgccgaaactttccgttccgtttttatatgcaaagt
aaagtcttacaaacagcataatgaagaacatcagcattgcacaaaacaaaccagacaatatattgttattccatgtctgatataacaggccggagaagataaa
tgtcacggtataaaggcgtggtagtagaaatgaaagaaggtctatacaggccattaatgtaaggaaagcatagaaaacaatcactattatggattgaatcagt
gatagctggtttaatgaaagatacagaacggcaagcgttgtcagtatcagacagtaatatttctctgtgccggttttataagatacattaagtttatttaaac
ggaattttacttcattgtaggcgtagcattgacagacgatatttgcacagcaaagtatcgccgtattaaatatgctgtttagcataagttaccttaccatgta
tttattgtagacatagcgtgcgtatttgattcttttctgttcgttactttctttgaaaccagcgttataactgccaagacaactccagtttatgccacatttc
tgaaaatgtgttgccagaatccacgcgccaatttgtatattgagacaggggttatcaataagctctgatttgtcttttattacgccaagttttttcaggttgg
ggatgtgtgatgaattaatttgcatcagtccgtaatcggtgcttaatatttttcctttttctttgttgtgatttatccggtctggtatgagtgaactttctac
agtggcatgcttttcagaagcaggggatcaatcagataacgttgcccggcttgtgcaaagcaggcatcaaatcgggtcgttgtcgctgaatatgcgggttgca
atgaaaagattaatgatgaaatacatgatagcaggatataaagaacagaacgcacaatactcctcctgtaaacatggaaaaaataagcgcccgaaggcgctta
tccgatgcacatgaaaaatcaggaatcagtgctgaaggtcagcgtattgctgtcagatgagcaaacagttgccgcatcgacagttgatgtggaagtgttgtta
atcttggagattgcgctggaagaacgcagggcgtttaccattgccatgcagtttttcttcggtacgctggcctcaacaacattgaaggagtatttatccgaag
aggttgtgattgtaacggagccgccccacgggtttttcgcgcttgcgccagttgtatctgcgatcatatcggatggcagtaatccttgcgcgtagagagtctt
aatatagttactgtcagtataacgcccctggaatttcagtgatttcatgttggcaatcactgtcaggacattgttttgttcattggatgattgaatgttggat
tgcaccattgaatagagtttatatgcacttgctgcaagtacaacaataacaccaacaaccaataacacttccatcagtgttgcccctttgtcctgttcttttt
tacggtgtgcagaaagagaagaaaaaacagaacgcatatttaaaatattaatagaagacataataaataattccttatttattgagaggttaaaaacattagg
gattagtgttgcaggacgttactgacatcgtagagagcagtaatcatcattacaataaatgcgaaaacaattaatgcggatactacagttatccagactcctg
ttgatttgacttttccttttgcttcctccagccatttatcagcatagttagacatcatcaatgcattgtcagcattttcagaatgaagtaatagtttgttaat
gcataatttcgatgggaagtcataaccgcaacttttcattgccattgcaagtgactgtccaagtaaaacatatctcctgatttcttttattcgttcgagtagc
catctgttttgagtgctgtttttctccagcctttccagtgcttttgcaacaggaatatcaatacttaacatggaggccatgttgaagagaaaacttacaccat
taaatgttttataaagtgaccaggggggaatgtaatcaagatactgacggcccggtcctgtataacgtgcaagcgaatatctgatcaggaaaaacagtacgat
gcagaagcagagaatatacgttccgttttctatcaggaaaccagacgtatctgtcagtaatcgcatagaacttgtccagcgttctttcggtgcgagagaaagg
aaaacaggtgttatggtatcgtggaccatatacatgtttgccagacaaccaaaaaccagtacggatggataaatcatcgacataaaaagagtcattgtcattt
cagaacgacttctgacaatttttatggcctgaactaatcccccggcaaggtcgccgcgaatttcaccgctggatattaattgatattcatcctcctttatata
tttttttaatgctgaagatattgcccctgtgttattcagtgcattaaggcactcacaacaaatatagtaggttgcagaagaccccgttttctttccgtagtct
gtttctacatcaatcaggtgtgtaagagcacgctttacatttatgccatcatttaacattccagataaatcttcatataaacttattctggaatacctgaatg
tgtattttgccagccagtgggatattccagagaaatcaatttctatcgaattgagtttctcaaatatatcttttatcgccatgtcccctccgggagcattgag
tatttatcttcgtcaatgggggatatgaagtgtgcggacagcggatctaccagaccatgattaatcaggtgtaataagtgctgattacgggtaattcctccaa
gttcatagtgccagtatgattttgcttccagacgcccggactgacgatataactgaaaaaaacgtgcgtcgggtgagacgacttctgctataaccgtccttcc
cgttacgcctttataacagtgctcgcaaccgtcaagattgcgcaaccggacctgttccggcaggcaattttcctcaatgagtttccgttcttctgtactcagt
ttgtctgccatttcagcataagtgcgcttacagtaaggacagagtttttgtaccagccgttgactcagcagaccgataaacagaagaggatctgcaatcagcc
tggcctgaatattctccatttcaaggcgctcaataatattcagggcatcgttggcgtgtaaggtcgtcaacagaaggtggcctgtcatcgaggctttgattgc
tgctaatgccgatgcgtgatcacgaatttcgccgttcagtatcgcgtctggatcaagtcgcatagcggatttaatcgcgtcaacccagccatcgacggaatcc
ctgactgccgtctgtattgcgccgggaatgcggccctccggcggtgattcaatggtgaaaaggcgttttcgggggagacgcatattatcgttatggttgaaac
caaacgtgctcagataggcttctgaagccgttctcagagtcgttgacttaccggaccctgtagggccggataaaacgataattccttccgggcgttgtaacat
tcgcctgattgtctggatttgttgtggcatgtagccaagttcttcgaaagtgggaatatcctcgctgtcatctttaatcaggcggaagaccgcgtacacacca
cccacgcacggataatgcgaatagcgcgccccaaaaagatttacaggcttcagaaaattctctgcgatacgtgcatcctgcggtattgccgggtcaaactgcg
tacctttgataacatcgctcattccagaataggcagcacccagcaattccagcccttctttacgcgggatgacatccagaatttccagttccccatgcacgcg
tgcttcgacataagtgaactcggaaccgtcccgaccaggtgtgatgtgcaaatcggaacttcccaggttgtttgctttcgccaggtagctaatgaccttttcc
tggttgctgtttaagtgttgtgatgagtttgaatcagaccttctttcgctgtcctggtagcgggatgcgataacatcaagagtggtccaggttatatttacct
ttccgggatatttaacattcagagtgccaatataagcctgaacagcacgctgtcctttactgttatcagcgatataaacatgaacaggtttatcgtctttttc
agatatactgacgtatacaacatcagaaatatcttttccgggatagtctgcgtcagaaaaaaacattatgtgtcgtctctttcaatagcattaaagaataagc
tggcgtttctctgtaccattctgaatccagacgccattaccagtgattgatgccaccttataagaagtaccaggaatactttgcccgaccttgacatcagtta
ctgacccgtccaggagagaaattcgtgctgacatttgttgtggtgtaccaaatatttcaataacacgaagtgatttagcggctacacgttgcgctggtatatc
ggtatctgcaagagaagaatacccggaagagggcgttattgctggcgttgcggacgctacaccattcaaatcaagattacgcaattcactgacagccttctga
cgcgccagctttgcttcgtaaattaccgtttcagcctgaatctgctctacctgtaaaatggtcggagatgaatattttatttcgtcttttggtgtgctttcag
attctgttgcgtaagcaagtgaactgaaaaaacagacagaaataatcgttggtttaattatttttaacatagaatgcacctttaatctcataattcagtcttc
cttcttccagttttattcctgcatactggaagcgaacggcaggttcatgaaagttagcgaaaagtattgaaggcggggtgctggtctgaatcattaattcata
ctcgttccacgggagaattattggacggccttcttcatctgtttttgtgttttcaatttcctgccaggtgagttttaacctcattttttgtgcaaatgttgtc
aggcgttcctgaatatctgttgcctgtggcaatgtgtccggggtaattggatcaggcgaaacgtcaaaactgacaggaagagaaaatcccccggcgctgcctt
ctggcaatacaaaagttgctgtggttgtcccctggaatatttctcgtatccgtgtgctgaagtcctcgaccgtaacgccgctcagttccttatatgatgtgcg
taacagcccgtcattaccggatgtgctgcattcagcaaggtcaaaacgccatccggcgatcgataagggcagtgcatcccatttatcaatgcatttatcaata
aatggttttatttcaggtaaatgttgccacggtggggcaatctgtattgcctgtttgtctgccatttcttttgccagacgaagacgtgcagcttctgccgcag
cttctttttcccggtattcctggtacatggtgatcccgttccagagcagtatggccagaatggcacttccgccataaatcatgaactgacgttttcgggatac
gcgagtaaacgccgcctctttcggcggtttttttacgtcaatcaacgcagacagagtgagtgaggggagtgcctggctgatatcccatgattcaggctggtat
agtttccagccgggttctggtgtttcattaaactcaagaaatgttgtcagagcactcatgatctgacttttgttacctacaacatcgttgacgagaacgttat
tgacgcaggaaacaaagccataaagatcaccgagttcgcagatagcgtatccgtcaggttcgatgaggggttttatcatcagtgccagtgaccagaatgagga
cgcccctgtgggaagatcctcttttctgataaatgccaccatcgttttggtttcattttctgaatcctgatactgacaggagagaaaatattcagcgtcgttt
tttcttgcgaatgtacgcgcattcttttcttctgaaggaagcgttttccagtccagaccggagaaaaaagcacgcttgtttttaataaccgaagtgaaattcc
ggttattctgattattctcagacaaagtgacctccaaaaaaactccctgtgttttttcagggagttttcagttaattagttcacattttttattactgcactg
gagccggaattaagcacatttggtgtaatcaaaacgaccagaatcgtgtcgccgtcttcgctgttaacgccacccccaagtccaaagaaggagggggttatta
caccttgcttactggttttccggtttgattgctggaatccactcagaatgactgtctggcctgatttcagatcaatagtctgattaattgttttcaaatccac
attcggtgtttgtgctttactgccgccactttcgaacacctcaaaagtaggtttgtcagacagactcattgagaacagcaattgcaggttttgtgaatccggc
tgaatgtatggaagcagagtcatattgaatccggttgttattgttgccgcattcagtgacgtggatgatcccacattggcggtagaatcggttgtgacacttt
caatgaagccctgctgattggcaatttgcatagggacaggcgtcaggtttttagtcacagcggagttttgcgttacgacactgacgctgccttgtgacgacag
ggctttcagaaatgcttttgagccagccagcttaccgtccacgatggatacaccactggtaataacattctcggcggcatcaccaaatgagccaccaagactc
agaccaagatgaccatcattaaatactgcgttccagtcgattcccgcctgttcctggcttgatttcttgatactcaggatttcaacgttcagcacaacctgac
gactcatttcattattgcgcttgttcacaatctcctgaacgctgtccagtacatccggggtatctgttactgtcagtgaacctgtggacagatacatgcgccc
tgtacctggtgtcagcattgaggaaacttcgcttttcaggtcgttgtacagcgatgatttcatttccacggttgtagtttgcgtatttgatgcgtcgccagtc
ataccgccagaacttgtagagccggtactgccactggaagacattgtgccgctcacaacttttgagttgtatgccacattgctgtccatatatgtaatcggga
atgagcgcgtttccagatagaagaatgtgatacgacctttatcgtatttccaggatattcccagccgtgaagatacggtttgcagtagggcaggaagcccgga
gatattcagttcagggagtgatgtaccgccagaagagacttttactggctgactgcctactgacgccagaggaaccattccatttgcatcaggtgctggaatt
gagcctgtcatttgttgtgttgagccagtgcctgtaccgttcagataggtccagacatcggcgctgattcgtaccgggatatgacaggtttgtgtgatgcgtt
gtgcgatctggcgtagctcaatatctgaacgagtattaatcacaatcgggcagggcggcgctgattgctttacctgcttttcaataattggcgttgtattaat
ccattgttttgttgaatcctggataaccgggttattacgaatttttttcgcgtattcagtcgctttttgtgcgtctctttcggcagcaagttcattagcattg
atttttttgaagtttgcacagcctgataataaaaaagaggatattaataaacaaagcaattttttattcatgtttaattaccttatggaatatgcaaaatact
atttgctggttgcgaggattattcagatacgtctgttttatcgtgattatcaaggtctggattagtaatcacattgcctccggcaaccatgactttttttcct
ccagctttagggagataaatttctgcaatttttgctatttcatccatgacttcttgtgtaaacaggtttggaatgtcaacatgattgttacgggcgatatcca
cgcggcgtttaaactctggttcttcaaaccagattattttgttggcaataaatggtcggctaaattcactggttataatttcacggattgcaatatttctggc
tttattcttcttatcccggagagacacaatttcttcaggaagaagaaccgggcgctctattacatcatcattccgtgtacgtgaacttcctcccttaccacca
ccactggatattgagcgtttggatattttcatatcacgcaccccaatctcttctgaaatctttttagccagagcattttttgatttcggcggatagtaaagca
caacagcactgttttcagtgaatgtggtccatccttcctgaccgtacatatcgcttttttgcccttgtccttcattctgaagaatgaacataaagcgaaggtt
ataacctgctgtaaaaccaacagcgcgttcaatgacctccgatttacccattgatgtaaattcatccaggagaataaggcactggtatttcaggtccgggtta
tgttccggcagttcacgacagttttcattaaccagtaatgagaagaaaaggtttacaattttctcatgagtgattaaggcatccggtgtcagaccaagataaa
ttgacatgggttttttacgaatatcccggatatcaaaatcactgaaattcgtcgcttcagcagtaacagggttgctgaatatattcagtggtgatgaaaagtt
cgtttttatacttccccttgtgcgatctggtgctgacatgaactcaaagaagaatgattttgttttatcgctaatccagctacgattttcaacttcctgtccc
atccatgcggcgagatcttttccattatcgggaatcgacgttttcagtatggcgctgatggaaacaaggacgtccggtgcattatgacctttggctttctggt
ccaggtgaaaacgctccttgtccagcaggtaaagccctaacccgacaaaaaggttgcgtgcagaatcagaccatataggatcatcacttgccgggataaggat
tgcggcaatttttgccaggtccgtttcacgaagtaaatcactgcgactgacgcaatccagaggattccagcggtgagagcgtatctgtcctttaatggcctgg
tcgatagtttccgcgtatccgggggcgcaaagagaaagcatttttgccctaattctttttgcctgaatccggctgataaaaaccagttttcaagtttgatatc
cagtatgaccatacttcctggatagtttacgcagttggggataacaatccccacccctttaccagaacgtgtcggggcataaagaattaaaaattgttgtccg
gcgaaataaataaattgttttttgtatctccccttgaacattttaccgatgagtattggtggatgtttgtaaggtgattttttatccgggaaaaaacctgatt
tactcaggtccatatcagttgcgagccgggcgtcaccgtagatgacttttttaggcataatcccaatcacgacaattaacatgaatatggcaaagaatattaa
tggaacgctgaatccaaaccatgccataaaaacgagttttttatattgtgggttgccagaaaatgttgagattacagaatggaatgtatcccacttgagcaaa
gacatatcaacggatgaatatcttaatgctgtgtatccaccgagatagtttgaggctataagcacacccacaagaagcagtaacaggaagagaattaaccccc
ccgtttttttgcatccatagttatctccagaatttacattgtgctcaaaaagcgatttttttcttcgggttcgtaatacagttcagtcattctggtatggttg
aaaaaaagaatgacatcaattgatgtttttatcgtgcgcatgatgaggtccatatcaacagtcataccaacatcagattgttttacaatacttgccagtcgtg
aaaatgaggcataagtattattagcatgaatggtactgattgagccttcatgccctgtattaagcgcctccagataactccatgcttcgttacccctaagttc
agcaagaaagatgtgatctggctttaatcgcatacacgcttcaatgatttccttcgcttcaacggtattttttttgtagaaaagccggatgtggtttggatgc
agcggcagagacatttccggtacatcttcaacggttatataacgccgttcaggtggaaagatatctgcaattgcctttgctatagttgtcttaccggagccag
tcccacctacgattaaaaaattaagacggtctttaactgcctctcttaaaaattgagctttagattgaccatctggacgccgggataattcatacaggtagcg
ttgccgttcagtcaggatggcttcgtgtttcgtcgcaatcctgacattatcaaaacgccctgttcggacataatcatctatagtaaagcgcgttaatgatggc
ttacggatagatataacaacgctgttattttctgttgcaggtggaatgatgatttgtccacgttccccaccgggaagaactaccgatgcaatgggattatcat
ggctaagtggtatttttatctttgataagttcgtcagagttttggcaagtgtcattaaattatcaaaagtcgcgtctggcgcatctttagattcccagccatt
tttcccttcaaaccatattgttcctggtttattcacagatatttctgtgacatgttcaatattaagaacagactggatacccgtcatatccagatatcgtctt
gcggttttactgttatctcgtggttgtgatgtattactggtatctctgatgttattcatatcaattgctttactgaattactgaagttcatatacggatgaaa
agtcgatatttcgcggaacaataacagacaatacagtcccctgattaatatatgccgtggggggaatatttatcgtattgtccagcgttgtttttgccatttc
ttttgttgcgtcagatgtgttttcgtaggtgatattgctgtttgagcctgtttgttgcgtgctgtttttcagaatatctaaaccatcaccaagtaatgacagc
gcaatgcaccaccaaaacgctgaccaaaaatgattatcgacccatgcaggaaggccggaagcgcccagactgtcagtgccaagtgcgccaatgcgcacattga
cattttcatctttgagcgttgtccagttaacgaacacacgggctactccctgtgtcatgactttattctgctccccgatcagtaatgctcctttacgggcaag
aagcacttcgccgttatccgaccagacgtctcgtgttaactgacacattgtgataccgggataagaggttacgattttcgttttgagcgcacaggataatgct
gttcccgcagataacagatatcggcggttcagcacaggggatacctttccgtctgcgtactgactgccctgtaaatcatctctttcctggtttttttcgcctg
gttcctgattgtcgatcttcaccattgtgtcgcccatcagttgtcggacggattttggcaatggttgtggttggtttggatctccactggcggtttggggagt
gccgttggctatcacattgacggctgtttgatcggccttttgtgatgctggcgctgacggggcattgtctgcctgtttctgttcttgctcctcctgtgctttc
ctgcggttagcggcatcgagtttttccttttcttcgatgtttttcatcattgttgtcagtgaaacgccattgttggtatttgtgaccagtgtctcgtcagact
tctcaagaggtgtttgctgaacgacctgttctcgtgaaagaactttaaacaagataatgatcacaaataacgcggcaatcagtaatatgatgaggattaatat
ttttttgggcgctgctttaccttttacttcgataattccgcgatttcccgcgtcattatttttttcatcatctttatttttcataactgaacttctctactcg
tgtttttccgtttgacgtaccttttttattccacggacgattaacaattgatgatgttctgatatctgcaacctgatcacccaggcgcaaacggagattttca
gccacgtcatagtaaaccattgtgtttttatttctgtctccgttaaccagatgttcatttccatcggcatcaacacgataaaggaccggaatttcctgtttat
tagtccatctcatacatgtaaattcgccattatcccatacctgataagggaaaattgatttgtcccccttgagttgataatgaccattaattattccaccatc
agtgcatggaaatggcctttcagtcattacggtagctgacggtggtttcggataattaaaacgcagaatatacgtgggttgttttgtggtcgatatcaggtag
agaggataaatatgtttatttgtctgaacggtaacgtttgtatccggttcttctgcttttggacgcagaaacagagtgttgtccctgaccgatactgaccagg
cttctggatcaccaattccgacatcttttattgtttcgtcttgtccgaacttaattgttgtttgtgcgcctgcttttacgcgcacattaaatacatcctgctc
gttataatctacggtctgaatacgcccgtctcgttccgatggtgtgccgtacatagctgctaaagcaccaggtacttgtagcatcgacaaaagaactatcgca
gtagtgcgtttaagcatcattttataacctctggatcaggttggtagctcagaacttgcagtcccagcggattcacaaggcgctctttttctgttttgatatc
tttatcccagtcaaaagaaatcgtcgaaatccactctgttgctctgtatccggggggggtttaccagataaatccagtaccatttttttgaaacgcacctgcg
ccatatccttccttagaaggattactgaattaatctggaccttaattttgtagttgtttttcagaatgttcagtggtgaggaattagcacgaatcatggtgtc
ataagctgaaaacaccttttgggaagacattgtttttactgtatcggcctgttcctggatagtctgccagtcgtatgcttcatagttaatgagatattttgac
aggaaatatttagtctcaacatcctgataactttccttagcatcagataattgaggggcaatatcagtatatccagtgctgttatccactcgcacaaggaacg
gtacagcggtttttaatggagcaagggaggctattgcgaaaccaagaatgcaaattacaacaaattcaccaatggcgagaatggtaaatgtttttgccttttt
ctttatgatttcaatttcactgcgctcaaagtcacgtatggccttgaaataatcctttttttcgttcttttcttcaaatttctttttggaggcttcttttttt
gaatgttcataatagtttgtgttttccattaataatcacctgctttgatttgtccaattgctgtgtttaactcaaaccatcccccttttttctgcacaggatt
gagggtgtgagaacatgcactaattaatagcgcggaggcaattatgaggggggcatattgttttccgttctgtataaaacagagaaattaaccgttcaggcgt
ttcaggagttctggtatccagatatcaggagctacatctcctttttcagcaataatctcatcaaaaatagacaggccagtttcagagccagaaagcacgggca
taaactcatcgaatccaaacaaatccattttggcgaaaacagaagagccagattgcttgatgagcatggtccttgagtccttcgccagctctttgagtttttc
aaactctttttcagtaagcccaatttctttatatccttcccattttgcatcgggattgggcaggagaattttcgttgcggtttgttgtaccagcgccgcaaaa
attgcgcagtttatggcgtcttccggggactgtgatgttaaccacatcatttcacctttcatgcgccccgctttcagtgcgcttttaatcatgctttgtgtca
tgggaaagtttgctggcatccagaactcttcaataatggagagcatcagattgccgccgcgttgcatgatttctttgtagaaaaacaggaccgcgagtaaagg
ttcacaggcaggatgaataccgttttttgtgtccaggacaacggttgtatcaaatcccacttttttatgatgcagtggattgaaggtattccggggggaatcc
accgcccaggcatattcgccgttgtcgcaccatttggcgagacgcgtctgaagttcaggactgactatatcgagaagaagggctgtccggcgttcttcgacag
gcattcgcataatgctttccacggcagcgtttaattcaatgccatgttcatcagaacaggggttgccctggttgtctcgtgccagaacctgtgtccagcgttt
cagaaatgcgagaagtctgtgccagacgggagattccgggcctgctgccagttgccacggattgcagcctgtataaatgccttcctgcaacgtgaaatagctt
cctccgtatgcacggacaaaaagctcagtagaacggttgaaatccacaacaaacattaaagggtcaaagcgttgaagaaaaccgctggctgctgcttcaaatg
ttgttttccctgtgcctgttgcgccaagaatcattccatgcccggcaattttttgtccggtaacatttttttccggtgggctgtaatgcgtattgaaccagta
aatgctgtcagacaccgttttcaacggcattatggcggacccatcaccaataggattgcctgattttttcccggaggaataattgtgaaaagacatcagacag
gccaggttggttattgttcttcgggtatccagtggacgcctcttattcagcggcatatgactgaaaaaaacaaaaggtgatgcgagtgaggcgcgactgaagc
ggaatcccttacccgaagtaataaactcagcagagagctttataccattgcttctggcctgatctggtgtatcaccgaataccgttaatgcgcagtgtagtga
accaaacaacgtaattccggctgcgaccgcttctttaccagcttcgagttcttcctgttgtgttttggctgcatcattagctgattgcagtttgtttaattgc
gaatcaatgttcttcagggtttttgtgggtgattcaaaaataaacgattgagtaagaataaattcatagggttgcttgagaagaaaatcccattgtccggggg
tcgtttctatagggaaatccttcaaaatataattcgttgcaaactttttactatccgattcgttgttacgcagttcaagaacatcagcaccaaaataccattc
agaatcacaaatactggatgataagggagtggatgataaaggaatcatgttatgttctgtatttagcaatagtgataaatagtctgccacctcactaatataa
gtattgtgaactgataaaacagaagcgttaaatggctgaagtgctttttgcgcctgttgtgtcatttcattcatgcgctcgattccggtatcaacatcatcgt
agggaatcccaaatgttaaataccatgttgatttgtagaatgcgctggatgtgaacagagcaaggtatttttttgaaaacctgcttaaaaaagcattgtcaaa
attccattctccatcaatggttattctttttttgataagatgagtccataaatacaggtcgccttcctttcctaacccaatcaggaagtttttaacgctgtta
aataaatttgtaagaacattatcgttctctgtctcaaatggaatgcctgctacaacaagtgaagccagcattttcctgtcagaagtaaagataatactgtctg
ttatatggaagcgataatcgggatattcacgagcaacatccatattgtcactgttttttaatttgattttgtgcatatatgttacctgacagatgaataacca
ataattaaatctctgtggcggatttttaacaaaagccccataaggtttaaacggatgactcttagtgcgtttgagtcattttcacaggccagttttactataa
aaagaaacagcgcacatatgacaggaataattatcccttttataccccaaaggaaaatagcggggaagcctgtcagaacagcgaaaaaagccgtgaacaataa
caacattaagggtataccagcaattaacgcaggacggttaaacccgttataagtcatgaatgactcactgacatgttcatttactttgtccataactcaccat
gaaaaaagccgcaattcacacataaggtaaatatgcggcacagttgtatatatggattacttatttgaagagttccagcgcccagttcccggcgtaaagtgca
ccgccagcgagggaacaatagataagcgccattccaacatcagaccagcttttcttctccatgaatgccatgatgatgttatagatcaggtaacacaaagcac
aaacaccaccaaagccaaatgcccatgtcttaagttctgtcatggtatttgttgcggtgtcgagtccacctgcataggcaacggaggcggttagtgacaggaa
aaacgccaggataaaatactgcaaagttgatttgactttcattaactaaaatctccaaaaacatcccagtccggtgatgttttctcttcatcattcgggggtt
ttatttctgattcatcagttttttcacggattgatggcacgatatagttattcgcgttactaataacccgttccagataagaagtgttgttaaatttcttttc
ttttataaatcctgttgattcgttacccgaataatacaaagataaagcgcggagaattttttgattactactaccctccttttgtctcagataattgtccgtc
aggactgccgctcctgcttttatatttgtacacgggtcaaaaactgttttattagtcagattgagtgatgggaaatttttgctgtatatctgtgtcagacctg
cactaaaccttttattttctgctgttaatttttctgcatattcaatagctcctttttcatctttaaaatacttacttgttccgtcagaaacattcgctattac
atatggattagcccctgattcagtcataatcagggcattcatcgtatcgggtgatacatcaggggcacactgacttatcagtgctgccagaacagccgctgat
aactgcataagggatttatctcctgatattaaattcgtgaagaacgttttttcctgcgggggcgacgaacgatactgtccagctcattagcgccggaaagaag
ttctttcagcgagacaattttttccctgacaggtataagggattttgcccccggacattgttcaaagtctgatttgtacaggtcgtataaaacaagcgtatgt
tttacatcctgtgacagataattatgtttattctggatgttaccttgttcgatatttttgataacatctgctgaacgatgaattaaatctgtgtagattcggg
tgtttttaaggggatttatttttatatattcagaagcaagctgaataagattgcatcgtaagataaggacttcacgagtggcaatatcaagctcgctgacatc
atcattatcctgcatgggtatctccggtaaagattttctggactgaagtccgcgccgtagcgatatatccggcccacataatgatgtgtcggatatgcagact
ccagttcagaatccccttgtcaggggatatttagttaatgagtacaatagcctcttctggaattgaggaaggaacgggaacgcccatatcattcccctgatta
tcgacaatcttcttattttttactcttcctaacattgctgaatgttttgtttgctgctgcaaagcagacatcagaccttctgtatcggcagaccagatataaa
ctcgcttaccgtaaataatatgatgaatatcataaacgggtgttataccgatttgctcagatgttaacgttcctcctgaattacgtttgtctggatatttata
aagatagtcattgataacatttgcgaaatggacaaattcaattgcttcctgttttgcaggaagcaacttcattcgctgtatggtttggttattgttatctgtc
agatagtaactgcctattgacaggaatatcaggagcaagctgaagaaccaacccatagcgattactcctgttgtgtgtctttaattgtgattacgcattgtgc
aatattcctgaatgcgtataacggtttatctgctgaaagataaagcatcagcgtatcttttaatgcagattcaaagtttccccggaaaataagaggagcatct
attctgtagttcacaggagtatcccatacaagattccaggttgaattaccacacggcgaacttgacgcccattctgtgaggccatcccgcagcgtcgaacctt
ttttaagattccaggtgagtttttctgtctgtttttgcggaacggtgacaggcgttagcgatttttgaggtaacttgtcagaggaaactggtgtcctgacagg
agatgcaggggttttgtcaggagatacaggtgctttttgtgatgttttccctttctgttgttgaaaatcaatcagaagatgtcttttttcggtgttcttaatt
accgtcagaccgtagttgcgaagcattttatccaggacataaggccattgatcgttgccctgccaggatatcgtgcgacggaaagttttcgttacctccgggg
atatttcagccgtccagtccgatgggacaatttcttttactgctttttgcagcgagcctttaccatcatggcgaacaaagtaaatttgcttttcaggtgttga
tgggcctgtaagcgtggtggccggaacagcgttgttagcgttttttttcagaaaaccgctttttgacattagcggtggttgttcaggacgcggttttgtctgc
tgatcagtattttgtgccgtttgtgcctgatggttgttgctactgattggctttgcgggagggagtgctggttgttcagaatgctgtttagttgctcctttgg
ctgctgtttttatctcaccagatgaagttatgctgtttgttgttggcttcagaaaactattattgctgtcctggttttttggcgctgtcgctgattgctcaga
acgtggttttacggtttctgcaatacgaggcgcgggagttacgtttatggcttcctttttgagataattatcattgctgtattgaacagtgctgtttggctgt
gacacgggctgctgaaggatcaccccgttatagtatgtgtctgctcttacctgctcaattgtggtattcagttcataccaacctccttttgcctgtgcgggtt
caggggcatgggaacacgcattcagcaataatgtggtgggaataataatgagctttttcatggttaaccccaggaacgtccgaacttactcatgatttttgag
atcgccgcagggtctgcggtcgtgcattgattaagaaacgcttttcgggcatcaaaggttttgctccagcgaatagatccctttttctttttgatgatattaa
agaagtccttttcggcgcttttgcattcgctcccgccgccgctaccgatagctttaccgtacagacaaagaacggaagcacacggatcagacgcatatacggg
ggcagagagtgcgatagcacttgcggcaatcatcgataacatgattttgcttaacggtgtctcaggcacggggtgtgttctactatctgcgcagcctggtcgt
aatgctgcggtggcggcaaccatcaataaaatggtggatttcatggtgttcctccatatttcagacaaaagaaaacccgcacgaggcgggtaatttaaatatc
aggggaatcaacgcacgtgtggcgtaatgatgacaatccggttctcactttggctatcagcattaactccagtaaaaccactcacgataaggggctggtccgg
tttgagtaaaatattctgctcgacagtctgtttccgtatcgggtcagtggttgtgagtagcaggtggaaatttccacgcacaatctccggtgctatccgcatt
gtgaagccagcagtccgatctttttcagccagtgtagtgtcaccggatattgcctgacgggggatcagttccgtggcaggttcccagcttccggcagatgttg
tttgtgattcaacgcacggggtactctgcattattttgcggattgtttcggaggatatcgcctgaccgtcatcaaagaatgcactacggaaaatcacgttctg
atttttggcaaacaagcctggatgccgctcgctcagactttcgtatatattcataccattcaaacaaacaggagaactgtccggcaggtctgttcgcacggta
tcaattttcaacgttatctggtggtcatttgaggttaacggtgactcaggtactggttgtgttttgctatccgcgcagcctgacagtaatgcggtggcggcaa
ccatcgataaaatgatggatttcatggagttcctccatatttcaggcaaaagaaaacccgcacgaggcgggctttgtgacggggggagaggagaacgttattg
tagacgttccattaattcatttactttcttcttgtgctcttctttttccttgtaggattgtccaagcatctgatttataatttcttttctcttgatatcatac
agtgcttcgttggcattcttacagttattatttgtaatttcaccagctttgcatttatcagatatcttttgggcttcatcctggtgttctttatagtaagaga
catcatagattttgtcctcacatccagttagcaagccacagaataaagctagagaaatgatagtacctttattcatggtatgtattccttaatcaaggcgtct
tctcatttcactaactttctttttatattcactatcttgtttttgctgaagtgcagcacgagcattatcgcagttttgatcggttattgtacctttcttgcaa
tcttctaaagttttagtggcttcactgatgttgtttgagtaatattctacagaatatattttttctttacatcccactaacattaaactggcgaagataattg
atgaggcgataattgcatttcgtgacttattcatatcagcttaaatctcttctcttttggcgaacatatgtttcatattgagctttccgctcattctctagtt
gccgttcagcaatatcatattgcttcaattgtagctccattgtcaatttttctgtattcaattggttagataagtcagcttttttttgcggagtatctgctga
cgcgaactccttctgtaacgtggttaatctctcagccctttcatgcatttcattgttgaaattttcatagaatttgatcttagccaggattgcatcatatttt
tgttgcgtctctgcaacgttgcttgataagtcatattgcgatcgcaaagagctaagatctgattttgcactatttgttattgttgatagagaatccttcatat
agctggatgcgctatcaaagccattgctgaaatcagtgaagccttcgagtcttttcttctgttcttcagcgtatcttatggtctgttcatattgttgcttgat
gttgctcaagctgtcttgccgcttcagcggcacgatttagcccttcttcaactgttttagctatcgccgaacgtctactactggaataccagacgcaaagatt
gatggtgatgttatggtaattattccagcaaagagaaatgataatactttatctttcatgttaatcaccccgctttaagtttacttccacggtttaatccctg
aactaatgctgatgcagaacgacttcctgcattaaagaatggatttggcattttgctggcaaaaccattggtgaaagatcggagtcctgatgctcgcataaaa
ccggatgcaaccttacctccaaagccattagcggctgatgtaaggccatttatacctaccccgccagtaagagttgagcagagagtacctacttgctcaacaa
ggaatatagatataacagtcaccataaagaaataaactactgttgtgagagttatatttccgcttgatgggactgtttgctctacaaagctgatgacgaagct
gaacgatatactataaaaaagatttagcaatatataatttaaacagctacctacccatgcgctaaacatatttcttgtcgaagcaaataaagaaaaacagata
aataatattccggcggataagagaatgccaaccatgaatgttgatagggttagaaatacagtggtgtaataaataagcagcagcccgccaacatatccaaccc
cccaaattagatatatgtatagttgatctgtgaagctataccactccagagtatctaactcattattcttaaaatcatttaacgttactgaaagcatattcca
caaattatcaacactggttgcagttcctgaacctccagtaacagcagcagaaaggtcggagccagcgtgcattacaaaaggaataacaaactggctgtaataa
ggcgctgaataagtgaatgcaccaaccagggcgaatgcaccgatattcttagtcacctcgctcatcaggacgtcacgttgggcatacgtgatttcatatatta
gataaataacgtataaggctatagccgcaaaaaatgtaggagatattgcctgtgcgaccttagcggcgttcgtcgccgctgtggattcaacaatggttgttac
tttctcaagaaattctgaaacaatgttcatagcttagtccttctggcagatgaagagttcaccagaggcactaatatttggcttcagcttaattaccaggtct
gtgccgaaaaaaattgcttctccaattttttggaaatttaatgtggattcttctttattgtaactataaacgttgtgggtcgctgtcgatgttttggttttac
gtggtgtaaacgcttttttaatgatgtaatcgagagcgtggctgttatgagatggtaaagtgattaaatcgtcgttttttttcataatgtagttctgattttt
gaggttgaagataattgtcggtcaatgcttttgattttttttcgtatctcaattggattcgcctcagataagttagcaatggataaagccagttcgatttgta
aaattttccttgtttcgatgagattgatttttacttgtggtatatcacggaagacaccacagcctgtaattgcttttgaaacgacacgttctgtattcgctat
gcttttctttactgttcgaggggggacataacgtgcacggcgaagttttctcagattagcttttgcttgagtcatatttgtatgccatgaataaataaatgct
ttcttcaaagcctgaataagcataaaccagtaatatggtggctaaaggcaaagaaaaggtgaaatgaataacggcattcagaactgaaccggttaatgtatat
aagacaaacggtgctgaaagtatgaaagcacccagcaggaaagaaaaaacattgcacgccatttttaataaaaatatttttattaggtgatgtttttttctgt
acatttcatcgtatccttcagggggctgtcaccttctcacgttgttgcttccatcgcgagagcttcaacaagttcattgatacaagtccgttctgccggaact
tcccgacctgtaaccgggtcgaaaacgatatgctcattttcaacgtaacgcaggcgatcgtagattttttgacgagcttttatcttaatcacgtccagctctg
catgttttaacttttctcgtctatcgccgatgaaaaatattatccatatgacagccatgataattgccgtacaggggagcatgtagcttaatacttttacagc
cacagcaaatgtatctttggcccttcctgctgctgattcaaggataaaaaaagctacagttattattatgactgtgtatgtctcgtatttatttacacctcta
ataccaaatttgcgtaatgtggctttagttacaatctctgaaagagaaacaccttcatttgcgtttcgcattgaattttcctcatactattcctgtgacactt
aacaccatcaataaaatatgtaatgcagtaatgttttttagcgtttttttgtaggcttcaataaaatccttatccagaagtctgataataaaaatacatagtg
cattcatcgctgctcctgtaagcggaattaacaacgaaagatgcatgatttattccttgttttcactcttgtcttcgatggtcattactttaagagttattcc
ttgcatgtctgtaacaataactcttgttcccgcagggtagttttcattgtgtaacgtctcaacaggccagaatgtatcaccaatgagtaattgccccttgccg
ttaacaatatcgtcactgagcgtaaattcttttcctttaaaacgtgaatgccctgtgttcaccaggagagtgtctaatttcttatgttttttgtcatagaaga
acttaattatcgacgccagtatgctgattgacgcaaagaaacatatattggcttcttgtgatatcggaagaaaaaagcccataatggcgcttgataatgcacc
aagactgattaagagtaaccatcccgttccggttataatttcaagtgcaatacacagaagaaacagtgtaaaccataacatggctaatctccatgataaaagg
cggagagtgaataaagataataaaggtggtaacaaacggctaatacattaaacaacagacataaaccgaagaatatgccattcaaactccatataagcagagg
catcgttgctatcatgagcgtcgaagatccccagtctagccatcttggaaatatggagtcgataatatgctgtagtctttctctttcattattatctcttgta
atagaaaggagaaaaaaccatgttgacatgggaattatatacaaaatgataatgatgcatgtataagttttcattttaatggctcactcaggagttaagataa
ggacggtgattaaaataatattgtcaccgtccgctatcttttttaagacaaatattgttgagcctgtttcatatcaactttgggagtatcctgtgatacgttt
tttagcagttcactgatacctgcaaccgaactcaccaggctacttgaatcaagcggcatcataaccagtttactgtttgatgctgtgccaattgcctggagtg
cttcagtgtatttctgcgcaataaagtaattaacagactgaacatcaccttcagcgattgcatccgacaccagttttgttgcgcgagcttccgcttctgcctg
acgttcgcgtgcttcagactgaagaaatgctgattgccgttccccttccgctttcagtatttgagactgtttttccccttctgccttcagaatttctgactgg
cgtataccttccgcttcaagaatccgggcacgcttagttcgttccgctttcatttgcgcgttcatcgcttcagtcagttcttttggtggtttaacatcccgaa
tttcaatacgtgtgaccttaattccccacgggtcagtcgcataatcgacaaccgttaacagtttagaattgatgctgtcacgttgagaaagcatatcgtcgag
attcattccaccaacgacagttcggatattggtcataacaagatttgaaatagctgatgcaagattatcgacttcataagctgccttcgctgcatcaatgacc
tggacaaaacaaacagcatcgatagttacgtttgcattgtctttcgaaataacttcctgtttaggaatatccaggacggtttccatcatgttgatccgttgac
cctatgcggtccataaacgggatcaggaagtgaagacgggggaaagcgtatgggtatatttaccaaagcgttccactgtccaggcattaccttgtggcacgat
tttgacggcagatttaacgaatatcagcgcaactaaaattaataagagtaacggaatagatgtaactattggcgtaattatagcgtcaatcatgattaatcct
ctcccaattcatcactgtggatgtatttctttgcaaatgaaatctgaaatatgaacatcaacagaaaaacactttcctgcttctttttcttttgagaagtttt
ttctgcattcttcagcggtc

### REFERENCES

Amor P A, Whitfield C. (1997) Molecular and functional analysis of genes required for expression of group IB K antigens in Escherichia coli: characterization of the his-region containing gene clusters for multiple cell-surface polysaccharides. Mol Microbiol. 26:145-161.
Baek C H, Wang S, Roland K L, Curtiss R 3rd. (2009) Leucine-responsive regulatory protein (Lrp) acts as a virulence repressor in Salmonella enterica serovar Typhimurium. J Bacteriol. 191:1278-1292. Bao Y, Zhang H, Huang X, Ma J, Logue C M, Nolan L K, Li G. (2018) O-specific polysaccharide confers lysozyme resistance to extraintestinal pathogenic Escherichia coli. Virulence. 9:666-680.
Barnes H J, Vaillancourt J, Gross W B. (2003) Colibacillosis, p. 631-652. In Y. M. Saif (ed.), Diseases of poultry. Iowa State University Press, Ames, IA.
Bertani G. (1951) Studies on lysogenesis. I. The mode of phage liberation by lysogenic Escherichia coli. J. Bacteriol. 62:293-300.
Braun V. (2005) Bacterial iron transport related to virulence. Contrib. Microbiol. 12:210-233. Clark-Curtiss J E, Curtiss R 3rd. (2018) Salmonella Vaccines: Conduits for Protective Antigens. J Immunol. 2018 Jan. 1; 200(1):39-48.
Cryz Jr S J, Que J O, Cross A S, Furer E. (1995) Synthesis and characterization of a polyvalent Escherichia coli O-polysaccharide-toxin A conjugate vaccine. Vaccine. 13:449-453 Curtiss R III, Porter S B, Munson M, Tinge S A, Hassan J O, Gentry-Weeks C, Kelly S M. (1991) Nonrecombinant and recombinant avirulent Salmonella live vaccines for poultry, p. 169-198. In L. C. Blankenship, J. S. Bailey, N. A. Cox, N. J. Stern, and R. J. Meinersmann (ed.), Colonization control of human bacterial enteric pathogens in poultry. Academic Press, Inc., New York, NY.
Curtiss 3^{rd} and Wang (2018) INDUCTION OF PROTECTIVE IMMUNITY AGAINST ANTIGENS. WO 2018/136938 A1
Dean C R, Goldberg J B. (2023) Efficacy of a Pseudomonas aeruginosa Serogroup 09 Vaccine. bioRxiv. 13:2023.07.13.548830.
Dho-Moulin M, Fairbrother J M (1999) Avian pathogenic Escherichia coli (APEC). Vet Res 30: 299-316.
Ding H F, Nakoneczna I, and Hsu H S. (1990) Protective immunity induced in mice by detoxified Salmonella lipopolysaccharide. J. Med. Microbiol. 31:95-102.
Doetkott D M, Nolan L K, Giddings C W, Berryhill D L (1996) Large plasmids of avian Escherichia coli isolates. Avian Dis 40: 927-930.
Dozois C M, Daigle F, Curtiss R 3rd. (2003) Identification of pathogen-specific and conserved genes expressed in vivo by an avian pathogenic Escherichia coli strain. Proc Natl Acad Sci USA 100: 247-252.
Dozois C M, Dho-Moulin M, Bree A, Fairbrother J M, Desautels C, Curtiss R 3rd. (2000) Relationship between the Tsh autotransporter and pathogenicity of avian Escherichia coli and localization and analysis of the Tsh genetic region. Infect. Immun. 68: 4145-4154.
Dziva F, Stevens M P. (2008) Colibacillosis in poultry: Unravelling the molecular basis of virulence of avian pathogenic Escherichia coli in their natural hosts. Avian Pathol. 37:355-366.
Fischbach M A, Lin H, Liu D R, Walsh C T. (2006) How pathogenic bacteria evade mammalian sabotage in the battle for iron. Nat. Chem. Biol. 2:132-138.
Ghunaim H, Abu-Madi M A, Kariyawasam S. (2014) Advances in vaccination against avian pathogenic Escherichia coli respiratory disease: Potentials and limitations. Vet. Microbiol. 172:13-22.
Griffiths E, Chart H, Stevenson P. (1988) High-affinity iron uptake systems and bacterial virulence, p. 121-137. In J. Roth (ed.), Virulence mechanisms of bacterial pathogens. ASM Press, Washington, DC.
Han Y, Liu O, Willias S, Liang K, Li P, Cheng A, Kong O. (2018) A bivalent vaccine derived from attenuated Salmonella expressing O-antigen polysaccharide provides protection against avian pathogenic Escherichia coli O1 and O2 infection. Vaccine. 36:1038-1046.
Han Y, Luo P, Chen Y, Xu J, Sun J, Guan C, Wang P, Chen M, Zhang X, Zhu Y, Zhu T, Zhai R, Cheng C, Song H. (2021) Regulated delayed attenuation improves vaccine efficacy in preventing infection from avian pathogenic Escherichia coli O78 and Salmonella typhimurium. Vet Microbiol. 254:109012.
Han Y, Liu O, Yi J, Liang K, Wei Y, Kong 0. (2017) A biologically conjugated polysaccharide vaccine delivered by attenuated Salmonella Typhimurium provides protection against challenge of avian pathogenic Escherichia coli O1 infection. Pathog Dis. 75:ftx102.
Hitchcock P J, Brown™. (1983) Morphological heterogeneity among Salmonella lipopolysaccharide chemotypes in silver-stained polyacrylamide gels. J Bacteriol. 154:269-277.
Johnson T J, Giddings C W, Horne S M, Gibbs P S, Wooley R E, et al. (2002) Location of increased serum survival gene and selected virulence traits on a conjugative R plasmid in an avian Escherichia coli isolate. Avian Dis 46: 342-352.
Johnson T J, Siek K E, Johnson S J, Nolan L K (2006) DNA sequence of a CoIV plasmid and revalence of selected plasmid-encoded virulence genes among avian Escherichia colistrains. J Bacteriol 188: 745-758.
Johnson T J, Skyberg J, Nolan L K (2004) Multiple antimicrobial resistance region of a putative virulence plasmid from an Escherichia coli isolate incriminated in avian colibacillosis. Avian Dis 48: 351-360.
Kaniga K, Delor I, Cornelis G R (1991) A wide-host-range suicide vector for improving reverse genetics in gram-negative bacteria: inactivation of the blaA gene of Yersinia enterocolitica. Gene 109:137-141.
Lerouge I, Vanderleyden J (2001) O-antigen structural variation: mechanisms and possible roles in animal/plant microbe interactions. FEMS Microbiol Rev 26: 17-47
Lewis G K (2007) Live-attenuated Salmonella as a prototype vaccine vector for passenger immunogens in humans: are we there yet?Expert Rev Vaccines. 2007 June; 6(3):431-40.
Mellata M, Ameiss K, Mo H, Curtiss R III (2010) Characterization of the Contribution to Virulence of Three Large Plasmids of Avian Pathogenic Escherichia coliX7122 (O78:K80:H9). Infect Immun 78:1528-1541.
Mellata M, Touchman J W, Curtiss R III (2009) Full Sequence and Comparative Analysis of the lasmid pAPEC-1 of Avian Pathogenic E. coli X⁷¹²² (O78:K80:H9). PLoS ONE 4(1): e4232. doi:10.1371/journal.pone.0004232
Moustafa D A, DiGiandomenico A, Raghuram V, Schulman M, Scarff J M, Davis M R Jr, Varga J J, Newman D M, Barbieri N L, de Oliveira A L, Willis D, Nolan L K, Logue C M (2021) Characterizing avian pathogenic Escherichia coli (APEC) from colibacillosis cases, 2018. PeerJ 9:e11025 DOI 10.7717/peerj.11025
Osborn M J, Wu H C (1980) Proteins of the outer membrane of gram-negative bacteria. Annu. Rev. Microbiol. 34: 369-422.
Pluschke G, Achtman M (1985) Antibodies to O-antigen of lipopolysaccharide are protective against neonatal infection with Escherichia coli K1. Infect Immun 49:365-70
Rodriguez-Siek K E, Giddings C W, Doetkott C, Johnson T J, Nolan L K (2005) Characterizing the APEC pathotype. Vet Res 36: 241-256.
Roland K. (1997) Live attenuated Salmonella vaccines to control avian pathogens. U.S. Pat. No. 6,399,074 B1
Roland K, Curtiss R III, Sizemore D (1999) Construction and evaluation of a delta cya delta crp Salmonella typhimurium strain expressing avian pathogenic Escherichia coli O78 LPS as a vaccine to prevent airsacculitis in chickens. Avian Dis. 43:429-441.
Russo T A, Johnson J R (2000) Proposal for a new inclusive designation for extraintestinal pathogenic isolates of Escherichia coli: ExPEC. J Infect Dis 181: 1753-1754.
Russo, T A, and Johnson, JR (2003). Medical and economic impact of extraintestinal infections due to Escherichia coli: focus on an increasingly important endemic problem. Microbes Infect 5, 449-456.
Schiff D E, Wass C A, Cryz S J J R, Cross A S, Kim K S (1993) Estimation of protective levels of anti-O-specific lipopolysaccharide immunoglobulin G antibody against experimental Escherichia coli infection. Infect Immun 61: 975-980
Schwyn B, Neilands J B. (1987) Universal chemical assay for the detection and determination of siderophores. Anal Biochem. 160:47-56.
Skyberg J A, Johnson T J, Johnson J R, Clabots C, Logue C M, Nolan L K. (2006) Acquisition of avian pathogenic Escherichia coliplasmids by a commensal E. coliisolate enhances its abilities to kill chicken embryos, grow in human urine, and colonize the murine kidney. Infect Immun 74: 6287-6292.
Smith H W, Huggins M B (1980) The association of the 018, K1 and H7 antigens and the Col V plasmid of a strain of Escherichia coliwith its virulence and immunogenicity. J Gen Microbiol 121: 387-400.
Stehling E G, Yano T, Brocchi M, da Silveira W D (2003) Characterization of a plasmid-encoded adhesin of an avian pathogenic Escherichia coli (APEC) strain isolated from a case of swollen head syndrome (SHS). Vet Microbiol 95:111-120.
Tivendale K A, Allen J L, Ginns C A, Crabb B S, Browning G F (2004) Association of iss and iucA, but not tsh, with plasmid-mediated virulence of avian pathogenic Escherichia coli. Infect Immun 72: 6554-6560.
Wooley R E, Gibbs P S, Brown T P, Glisson J R, Steffens W L, et al. (1998) Colonization of the chicken trachea by an avirulent avian Escherichia coli transformed with plasmid pHK11. Avian Dis 42: 194-198.
Zhang X, Goebel E M, Rodriguez M E, Preston A, Harvill E T (2009) The O antigen is a critical antigen for the development of a protective immune response to Bordetella parapertussis. Infect Immun. 77:5050-5058.
Zhang X, Kelly S M, Bollen W S, Curtiss R III (1997) Characterization and immunogenicity of Salmonella typhimurium SL1344 and UK-1 Acrp and Acdt deletion mutants. Infect. Immun. 65:5381-5387.

1. A recombinant Salmonella bacterium, wherein the bacterium expresses lipopolysaccharide (LPS) O78 antigen of avian pathogenic Escherichia coli (APEC) and does not express Salmonella LPS O4 antigen.

2. The recombinant Salmonella bacterium of claim 1, wherein the bacterium comprises APEC's rfb_{O78} gene cluster.

3. The recombinant Salmonella bacterium of claim 1, further comprising a deletion mutation in the Salmonella rfb_{O4} gene cluster.

4. The recombinant Salmonella bacterium of claim 2, wherein the APEC's rfb_{O78} gene cluster replaces the entire Salmonella rfb_{O4} gene cluster in the genome of the Salmonella bacterium.

5. The recombinant Salmonella bacterium of claim 2, wherein the rfb_{O4} gene cluster comprises a nucleic acid sequence having at least 85%, at least 90%, or at least 95% sequence identity to the sequence of SEQ ID NO: 4 or wherein the rfb_{O4} gene cluster comprises the nucleic acid sequence of SEQ ID NO: 4.

6. The recombinant Salmonella bacterium of claim 1, wherein:
(i) the recombinant Salmonella bacterium further expresses one or more APEC virulence factors;
(ii) the recombinant Salmonella bacterium comprises one or more nucleotides encoding one or more APEC virulence factors; and/or
(iii) the recombinant Salmonella bacterium comprises one or more APEC plasmids that comprises nucleotides encoding one or more APEC virulence factors.

7. The recombinant Salmonella bacterium of claim 6, wherein the APEC plasmid is plasmid pAPEC-1, pAPEC-2, pAPEC-3, or a combination thereof, derived from APEC strain X⁷¹²².

8. The recombinant Salmonella bacterium of claim 7, comprising all three plasmids pAPEC-1, pAPEC-2 and pAPEC-3.

9. The recombinant Salmonella bacterium of claim 7, wherein:
(i) the pAPEC-1 plasmid comprises a nucleic acid sequence having at least 85%, at least 90%, or at least 95% sequence identity to the sequence of SEQ ID NO: 1;
(ii) the pAPEC-2 plasmid comprises a nucleic acid sequence having at least 85%, at least 90%, or at least 95% sequence identity to the sequence of SEQ ID NO: 2; and/or
(iii) the pAPEC-3 plasmid comprises a nucleic acid sequence having at least 85%, at least 90%, or at least 95% sequence identity to the sequence of SEQ ID NO: 3.

10. The recombinant Salmonella bacterium of claim 1, further comprising:
a deletion mutation in the lrp gene (Δlrp);
an asd gene (asd+); and/or
a pmi gene (pmi+).

11. The recombinant Salmonella bacterium of claim 1, wherein the bacterium further comprises, or is derived from a parent Salmonella bacterium strain comprising:
a araC P_{araBAD}-regulated murA gene (ΔP_{murA}::TT araC P_{araBAD} murA deletion-insertion mutation);
an insertion of a c2 gene operably linked to an arabinose regulatable promoter araC P_{araBAD} (araC P_{araBAD} c2);
a deletion mutation in the gmd and a deletion in the fcl genes (Δ(gmd-fcl)); and a deletion-insertion mutation that inactivates the expression of a relA gene and inserts a lacl gene (ΔrelA::araC P_{araBAD} lacl TT deletion-insertion mutation).

12. The recombinant Salmonella bacterium of claim 1, wherein the bacterium further comprises:
an araC P_{araBAD}-regulated murA gene (ΔP_{murA}::TT araC P_{araBAD} murA deletion-insertion mutation);
an insertion of a c2 gene operably linked to an arabinose regulatable promoter araC P_{araBAD} (araC P_{araBAD} c2);
a deletion mutation of the gene cluster wza-wcaM (Δ(wza-wcaM));
a deletion-insertion mutation that inactivates the expression of a relA gene and inserts a lacl gene (ΔrelA::araC P_{araBAD} lacl TT deletion-insertion mutation)
a deletion mutation in the recF gene (ΔrecF);
a deletion mutation in the sifA gene (ΔsifA);
a deletion mutation in the waaL gene (ΔwaaL46); and
a deletion in a pagL gene and an insertion of a rhaRS P_{rhaBAD}-regulated waaL gene (ΔpagL::TT rhaRS P_{rhaBAD} waaL).

13. The recombinant Salmonella bacterium of claim 1, wherein the bacterium is a Salmonella enterica bacterium.

14. The recombinant Salmonella bacterium of claim 1, wherein the bacterium is a Salmonella enterica subsp. enterica serovar Paratyphi A bacterium, a Salmonella enterica subsp. enterica serovar Enteritidis bacterium, a Salmonella enterica subsp. enterica serovar Typhi bacterium, a Salmonella enterica subsp. enterica serovar Typhimurium bacterium, Salmonella enterica subsp. enterica serovar Dublin, Salmonella Pullorum, Salmonella Gallinarum, or Salmonella enterica subsp. enterica serovar Choleraesuis.

15. A pharmaceutical composition comprising the recombinant Salmonella bacterium of claim 1, and a pharmaceutically acceptable carrier.

16. A vaccine comprising the recombinant Salmonella bacterium of claim 1.

17. A method for eliciting an immune response against APEC in a subject, or vaccinating a subject against APEC, the method comprising administering to the subject an effective amount of the pharmaceutical composition of claim 15.

18. The method of claim 17, wherein the subject is a chicken, turkey, goose, or duck.

19. The method of claim 17, wherein the pharmaceutical composition is administered by spray or oral immunization.

20. A method for producing the recombinant Salmonella bacterium of claim 1, the method comprising culturing the bacterium in media.
